(19) United States
(12) Patent Application Publication
(10) Pub. No.: US 2020/0347043 A1
(43) Pub. Date: Nov. 5, 2020

(54) MODULATORS OF THE INTEGRATED STRESS PATHWAY

(71) Applicants: Calico Life Sciences LLC, South San Francisco, CA (US); AbbVie Inc., North Chicago, IL (US); SYGNATURE DISCOVERY LIMITED, Nottingham (GB)

(72) Inventors: Kathleen Ann Martin, San Francisco, CA (US); Carmela Sidrauski, Saratoga, CA (US); Lei Shi, Vernon Hills, IL (US); Kathleen J. Murauski, Libertyville, IL (US); Xiangdong Xu, Vernon Hills, IL (US); Yunsong Tong, Libertyville, IL (US); John T. Randolph, Libertyville, IL (US); Michael J. Dart, Highland Park, IL (US); Hanae Benelkebir, Nottingham (GB); Steven Edeson, Nottingham (GB); Kathryn Starbuck, Nottingham (GB)

(21) Appl. No.: 16/863,747

(22) Filed: Apr. 30, 2020

Related U.S. Application Data
(60) Provisional application No. 62/840,960, filed on Apr. 30, 2019.

(51) Int. Cl. C07D 413/12; A61P 25/28; A61K 45/06; A61K 31/422; C07D 405/12; A61K 31/4155; A61K 31/403; C07D 311/24; A61K 31/353; C07D 311/22; A61K 31/4433; A61K 31/4245; C07C 235/36; A61K 31/165; C07D 493/08; A61K 31/4025; C07C 235/40; C07D 405/06; A61K 31/397; C07D 407/12; C07D 271/10; C07D 471/04; A61K 31/437

(52) U.S. Cl. CPC C07D 413/12; C07C 2602/38; A61K 45/06; A61K 31/422; C07D 405/12; A61K 31/4155; A61K 31/403; C07D 311/24; A61K 31/353; C07D 311/22; A61K 31/4433; A61K 31/4245; C07C 235/36; A61K 31/165; C07D 493/08; A61K 31/4025; C07C 235/40; C07D 405/06; A61K 31/397; C07D 407/12; C07D 271/10; C07D 471/04; A61K 31/437; C07C 2601/04; C07C 2602/44; A61P 25/28

(57) ABSTRACT

Provided herein are compounds, compositions, and methods useful for modulating the integrated stress response (ISR) and for treating related diseases, disorders and conditions.

### CROSS REFERENCE TO RELATED APPLICATIONS

This application claims the benefit of, and priority to, U.S. Provisional Application No. 62/840,960, filed on Apr. 30, 2019, the content of which is hereby incorporated by reference in its entirety.

### BACKGROUND

In metazoa, diverse stress signals converge at a single phosphorylation event at serine 51 of a common effector, the translation initiation factor eIF2α. This step is carried out by four eIF2α kinases in mammalian cells: PERK, which responds to an accumulation of unfolded proteins in the endoplasmic reticulum (ER), GCN2 to amino acid starvation and UV light, PKR to viral infection and metabolic stress, and HRI to heme deficiency. This collection of signaling pathways has been termed the “integrated stress response” (ISR), as they converge on the same molecular event. eIF2α phosphorylation results in an attenuation of translation with consequences that allow cells to cope with the varied stresses (Wek, R. C. et al, Biochem Soc Trans (2006) 34(Pt 1):7-11).

eIF2 (which is comprised of three subunits, α, β and γ) binds GTP and the initiator Met-tRNA to form the ternary complex (eIF2-GTP-Met-tRNAᵢ), which, in turn, associates with the 40S ribosomal subunit scanning the 5′UTR of mRNAs to select the initiating AUG codon. Upon phosphorylation of its α-subunit, eIF2 becomes a competitive inhibitor of its GTP-exchange factor (GEF), eIF2B (Hinnebusch, A. G. and Lorsch, J. R. Cold Spring Harbor Perspect Biol (2012) 4(10)). The tight and nonproductive binding of phosphorylated eIF2 to eIF2B prevents loading of the eIF2 complex with GTP, thus blocking ternary complex formation and reducing translation initiation (Krishnamoorthy, T. et al, Mol Cell Biol (2001) 21(15):5018-5030). Because eIF2B is less abundant than eIF2, phosphorylation of only a small fraction of the total eIF2 has a dramatic impact on eIF2B activity in cells.

eIF2B is a complex molecular machine, composed of five different subunits, eIF2B1 through eIF2B5. eIF2B5 catalyzes the GDP/GTP exchange reaction and, together with a partially homologous subunit eIF2B3, constitutes the “catalytic core” (Williams, D. D. et al, J Biol Chem (2001) 276:24697-24703). The three remaining subunits (eIF2B1, eIF2B2, and eIF2B4) are also highly homologous to one another and form a “regulatory sub-complex” that provides binding sites for eIF2B's substrate eIF2 (Dev, K. et al, Mol Cell Biol(2010) 30:5218-5233). The exchange of GDP with GTP in eIF2 is catalyzed by its dedicated guanine nucleotide exchange factor (GEF) eIF2B. eIF2B exists as a decamer (B1₂ B2₂ B3₂ B4₂ B5₂) or dimer of two pentamers in cells (Gordiyenko, Y. et al, Nat Commun (2014) 5:3902; Wortham, N. C. et al, FASEB J (2014) 28:2225-2237). Molecules such as ISRIB interact with and stabilize the eIF2B dimer conformation, thereby enhancing intrinsic GEF activity and making cells less sensitive to the cellular effects of phosphorylation of eIF2α (Sidrauski, C. et al, eLife (2015) e07314; Sekine, Y. et al, Science (2015) 348:1027-1030). As such, small molecule therapeutics that can modulate eIF2B activity may have the potential to attenuate the PERK branch of the UPR and the overall ISR, and therefore may be used in the prevention and/or treatment of various diseases, such as a neurodegenerative disease, a leukodystrophy, cancer, an inflammatory disease, a musculoskeletal disease, or a metabolic disease.

### SUMMARY OF THE INVENTION

The present disclosure is directed, at least in part, to compounds, compositions, and methods for the modulation of eIF2B (e.g., activation of eIF2B) and the attenuation of the ISR signaling pathway. In some embodiments, disclosed herein is an eIF2B modulator (e.g., an eIF2B activator) comprising a compound of Formula (I), Formula (II), Formula (III-a) or Formula (III-b), or a pharmaceutically acceptable salt, solvate, hydrate, tautomer, N-oxide, or stereoisomer thereof. In other embodiments, disclosed herein are methods of using a compound of Formula (I), Formula (II), Formula (III-a) or Formula (III-b), or a pharmaceutically acceptable salt, solvate, hydrate, tautomer, N-oxide, or stereoisomer thereof for the treatment of a disease or disorder, e.g., a neurodegenerative disease, a leukodystrophy, cancer, an inflammatory disease, a musculoskeletal disease, a metabolic disease, or a disease or disorder associated with impaired function of eIF2B or components in the ISR pathway (e.g., eIF2 pathway).

For example, disclosed herein is a compound of Formula (I):

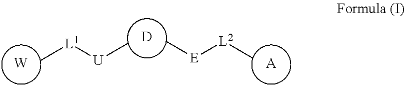

or a pharmaceutically acceptable salt, solvate, hydrate, tautomer, N-oxide, or stereoisomer thereof, wherein:

D is a 4-6-membered monocyclic cycloalkyl, a 4-6-membered monocyclic heterocyclyl, a bridged bicyclic cycloalkyl, a bridged bicyclic heterocyclyl, or cubanyl, wherein each 4-6-membered monocyclic cycloalkyl, 4-6-membered monocyclic heterocyclyl, bridged bicyclic cycloalkyl, bridged bicyclic heterocyclyl, or cubanyl is optionally substituted on one or more available carbons with 1-4 R^{X}; and wherein if the 4-6-membered monocyclic heterocyclyl or bridged bicyclic heterocyclyl contains a substitutable nitrogen moiety, the substitutable nitrogen may be optionally substituted by R^{N1};

U is —NR¹C(O)— or —C(O)NR¹—;

E is a bond, —NR²C(O)—, —C(O)NR²—, 5-6-membered heteroaryl or 5-6-membered heterocyclyl; wherein 5-6-membered heteroaryl or 5-6-membered heterocyclyl is optionally substituted on one or more available carbons with 1-5 R^{G}; and wherein if the 5-6-membered heteroaryl or 5-6-membered heterocyclyl contains a substitutable nitrogen moiety, the substitutable nitrogen may be optionally substituted by R^{N2}; or

E is

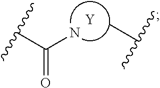

Y is a 4-9-membered nitrogen-containing monocyclic, bridged bicyclic, fused bicyclic or spirocyclic heterocyclyl, wherein the 4-9-membered nitrogen-containing monocyclic, bridged bicyclic, fused bicyclic or spirocyclic heterocyclyl is optionally substituted on one or more available carbons with 1-5 R^{G}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2};

L¹ is a bond, C₁-C₆ alkylene, 2-7 membered heteroalkylene, —NR^{N3}—, or —O—, wherein C₁-C₆ alkylene or 2-7 membered heteroalkylene is optionally substituted with 1-5 R^{L1};

L² is a bond, C₁-C₆ alkylene, 2-7 membered heteroalkylene, or —O—, wherein C₁-C₆ alkylene or 2-7 membered heteroalkylene is optionally substituted with 1-5 R^{L2};

R¹ is hydrogen or C₁-C₆ alkyl;

R² is hydrogen or C₁-C₆ alkyl;

W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available saturated carbons with 1-4 R^{W1}; wherein the phenyl or heteroaryl may optionally be substituted on one or more available unsaturated carbons with 1-4 R^{W2}; wherein if the heterocyclyl contains a substitutable nitrogen moiety, the substitutable nitrogen may optionally be substituted with R^{N4}; and wherein W is attached to L² through an available saturated carbon or nitrogen atom within the heterocyclyl;

A is C₃-C₆ cycloalkyl, phenyl, 4-6-membered heterocyclyl, 5-6-membered heteroaryl, or 8-10-membered bicyclic heteroaryl, wherein C₃-C₆ cycloalkyl, phenyl, 4-6-membered heterocyclyl, 5-6-membered heteroaryl, or 8-10-membered bicyclic heteroaryl is optionally substituted on one or more available carbons with 1-5 R^{Y}; and wherein if the 5-6-membered heteroaryl or 8-10-membered bicyclic heteroaryl contains a substitutable nitrogen moiety, the substitutable nitrogen may be optionally substituted by R^{N5};

each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)R^{D};

R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N3} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N4} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B}R^{C} and —C(O)OR^{D};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O₂)NR^{B}R^{C};

R^{N5} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A}, —NR^{B}R^{C}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or 2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};

each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, halo-C₁-C₆ alkoxy-C₁-C₆ alkyl, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or

2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};

each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};

each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, and —S(O)₂R^{D};

R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};

each of R^{B} and R^{C} is independently hydrogen or C₁-C₆ alkyl;

R^{B} and R^{C} together with the atom to which they are attached form a 3-7-membered heterocyclyl ring optionally substituted with 1-3 R^{Z};

each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D} is independently C₁-C₆ alkyl or halo-C₁-C₆ alkyl;

each R^{E} is independently hydrogen, C₁-C₆ alkyl, or halo-C₁-C₆ alkyl;

each R^{F} is independently hydrogen, C₁-C₆ alkyl, or halo;

each R^{G} is independently hydrogen, C₁-C₆ alkyl, halo or oxo; and

m is 1 when R^{F} is hydrogen or C₁-C₆ alkyl, 3 when R^{F} is C₁-C₆ alkyl, or 5 when R^{F} is halo.

Also disclosed is a compound of Formula (II):

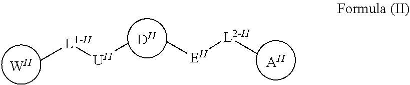

or a pharmaceutically acceptable salt, solvate, hydrate, tautomer, N-oxide, or stereoisomer thereof, wherein:

D^{II} is a bridged bicyclic cycloalkyl, a bridged bicyclic heterocyclyl, a 4-6-membered monocyclic cycloalkyl, a 4-6-membered monocyclic heterocyclyl, or cubanyl, wherein each bridged bicyclic cycloalkyl, bridged bicyclic heterocyclyl, 4-6-membered monocyclic cycloalkyl, 4-6-membered monocyclic heterocyclyl, or cubanyl is optionally substituted on one or more available carbons with 1-4 R^{X-II}; and wherein if the 4-6-membered monocyclic heterocyclyl or bridged bicyclic heterocyclyl contains a substitutable nitrogen moiety, the substitutable nitrogen may be optionally substituted by R^{N1-II};

U^{II} is —NR^{1-II}C(O)— or —C(O)NR^{1-II}—;

E^{II} is a bond, —NR^{2-II}C(O)—, —C(O)NR^{2-II}—, 5-6-membered heteroaryl or 5-6-membered heterocyclyl; wherein 5-6-membered heteroaryl or 5-6-membered heterocyclyl is optionally substituted on one or more available carbons with 1-5 R^{G-II}; and wherein if the 5-6-membered heteroaryl or 5-6-membered heterocyclyl contains a substitutable nitrogen moiety, the substitutable nitrogen may be optionally substituted by R^{N2-II}; or

E^{II} is

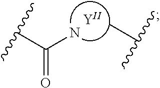

Y^{II} is a 4-9-membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{G-II}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2-II};

L^{1-II} is a bond, C₁-C₆ alkylene, 2-7 membered heteroalkylene, —NR^{N3-II}, or —O—, wherein C₁-C₆ alkylene or 2-7 membered heteroalkylene is optionally substituted with 1-5 R^{L1-II};

L^{2-II} is a bond, C₁-C₆ alkylene, or 2-7 membered heteroalkylene, —O—, wherein C₁-C₆ alkylene or 2-7 membered heteroalkylene is optionally substituted with 1-5 R^{L2-II};

R^{1-II} is hydrogen or C₁-C₆ alkyl;

R^{2-II} is hydrogen or C₁-C₆ alkyl;

W^{II} is phenyl or 5-6-membered heteroaryl; wherein phenyl or 5-6-membered heteroaryl is optionally substituted with 1-5 R^{W-II} and wherein if the 5-6-membered heteroaryl contains a substitutable nitrogen moiety, the substitutable nitrogen may be optionally substituted by R^{N4-II};

A^{II} is C₃-C₆ cycloalkyl, phenyl, or 5-6-membered heteroaryl, wherein C₃-C₆ cycloalkyl, phenyl, or 5-6-membered heteroaryl is optionally substituted on one or more available carbons with 1-5 R^{Y-II}; and wherein if the 5-6-membered heteroaryl contains a substitutable nitrogen moiety, the substitutable nitrogen may be optionally substituted by R^{N5-II};

each R^{L1-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};

each R^{L2-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};

R^{N1-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N2-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N3-II} is selected from the group consisting of hydrogen. C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N4-II} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B-II}R^{C-II} and —C(O)OR^{D-II};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w-II}C₁₋₆ alkyl (wherein w-II is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O₂)NR^{B-II}R^{C-II};

R^{N5-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

each R^{W-II} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, oxo, C═N—OH, halo, cyano, —OR^{A-II}, —NR^{B-II}R^{C-II}, —NR^{B-II}R^{CC-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II}; or

2 R^{W-II} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-II};

each R^{X-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-III}, —C(O)OH, —C(O)OR^{D-II}, —SR^{E-II}, —S(O)R^{D-II}, and —S(O)₂R^{D-II};

each R^{Y-II} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —S(R^{F-II})_{m-II}, —S(O)R^{D-II}, —S(O)₂R^{D-II}, and G^{1-II}; or

2 R^{Y-II} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-II};

each G^{1-II} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-II};

each R^{Z-II} is independently selected from the group consisting of C₁-C₆ alkyl, hydroxy-C₁-C₆ alkyl, halo-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, and —S(O)₂R^{D-II};

R^{A-II} is, at each occurrence, independently hydrogen, C₁-C₆ alkyl, halo-C₁-C₆ alkyl, —C(O)NR^{B-II}R^{C-II}, —C(O)R^{D-II}, or —C(O)OR^{D-II};

each of R^{B-II} and R^{C-II} is independently hydrogen or C₁-C₆ alkyl; R^{B-II} and R^{C-II} together with the atom to which they are attached form a 3-7-membered heterocyclyl ring optionally substituted with 1-3 R^{Z-II};

each R^{CC-II} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D-II} is independently C₁-C₆ alkyl or halo-C₁-C₆ alkyl;

each R^{E-II} is independently hydrogen, C₁-C₆ alkyl, or halo-C₁-C₆ alkyl;

each R^{F-II} is independently hydrogen, C₁-C₆ alkyl, or halo; and

each R^{G-II} is independently hydrogen, C₁-C₆ alkyl, halo or oxo;

provided that when D^{II} is a bridged bicyclic 5-membered cycloalkyl, E^{II} is —NR^{2-II}C(O)—.

Also disclosed is a compound represented by Formula (IIIa) or Formula (IIIb):

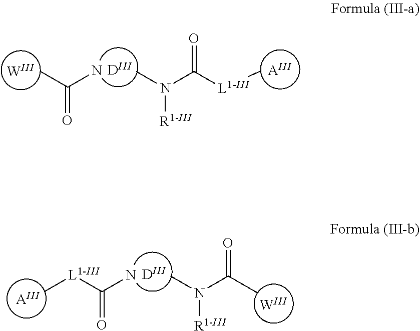

or a pharmaceutically acceptable salt, solvate, hydrate, tautomer, N-oxide, or stereoisomer thereof, wherein:

D^{III} is a 4-9 membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{X-II}; and wherein if the 4-9 membered nitrogen-containing monocyclic, bridged bicyclic, fused bicyclic or spirocyclic heterocyclyl contains a substitutable nitrogen moiety, the substitutable nitrogen may be optionally substituted by R^{N1-II};

W^{III} is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available saturated carbons with 1-4 R^{W1-III}; wherein the phenyl or heteroaryl may optionally be substituted on one or more available unsaturated carbons with 1-4 R^{W2-III}; and wherein if the heterocyclyl contains a substitutable nitrogen moiety, the substitutable nitrogen may optionally be substituted with R^{N2-III}.

A^{III} is phenyl or 5-6-membered heteroaryl, wherein phenyl or 5-6-membered heteroaryl is optionally substituted on one or more available carbons with 1-5 R^{Y-III}; and wherein if the 5-6-membered heteroaryl contains a substitutable nitrogen moiety, the substitutable nitrogen may be optionally substituted by R^{N3-III};

R^{1-III} is hydrogen or C₁-C₆ alkyl;

L^{1-III} is a bond, C₁-C₆ alkylene or 2-7 membered heteroalkylene, wherein C₁-C₆ alkylene or 2-7 membered heteroalkylene is optionally substituted with 1-5 R^{L1-III};

each R^{L1-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

R^{N1-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};

R^{N2-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};

R^{N3-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III} and —S(O)₂R^{D-III};

each R^{W1-III} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A-III}, —NR^{B-III} R^{C-III}, —NR^{B-III}R^{CC-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

each R^{W2-III} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —S(R^{F-III})_{m-III}, —S(O)R^{D-III}, and —S(O)₂R^{D-III}; or

2 R^{W2-III} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-III};

each R^{X-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

each R^{Y-III} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —S(R^{F-III})_{m-III}, —S(O)R^{D-III}, —S(O)₂R^{D-III}, and G^{1-III}; or

2 R^{Y-III} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-III};

each G^{1-III} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-III};

each R^{Z-III} is independently selected from the group consisting of C₁-C₆ alkyl, hydroxy-C₁-C₆ alkyl, halo-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, and —S(O)₂R^{D-III};

R^{A-III} is, at each occurrence, independently hydrogen, C₁-C₆ alkyl, halo-C₁-C₆ alkyl, —C(O)NR^{B-III}R^{C-III}, —C(O)R^{D-III}, or —C(O)OR^{D-III};

each of R^{B-III} and R^{C-III} is independently hydrogen or C₁-C₆ alkyl; or

R^{B-III} and R^{C-III} together with the atom to which they are attached form a 3-7-membered heterocyclyl ring optionally substituted with 1-3 R^{Z-III};

each R^{CC-III} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D-III} is independently C₁-C₆ alkyl, hydroxy-C₁-C₆ alkyl, or halo-C₁-C₆ alkyl;

each R^{E-III} is independently hydrogen, C₁-C₆ alkyl, or halo-C₁-C₆ alkyl;

each R^{F-III} is independently hydrogen, C₁-C₆ alkyl, or halo; and

m^{III} is 1 when R^{F-III} is hydrogen or C₁-C₆ alkyl, 3 when R^{F-III} is C₁-C₆ alkyl, or 5 when R^{F-III} is halo.

In some embodiments, a compound disclosed herein is selected from a compound set forth in Table 1 or Table 2, or a pharmaceutically acceptable salt, solvate, hydrate, tautomer, N-oxide or stereoisomer thereof.

In some embodiments, a compound disclosed herein, or a pharmaceutically acceptable salt thereof is formulated as a pharmaceutically acceptable composition comprising a disclosed compound and a pharmaceutically acceptable carrier.

In another aspect, the present invention features a method of treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, or a mitochondrial disease or a disease or disorder associated with impaired function of eIF2B or components in the ISR pathway (e.g., eIF2 pathway) in a subject, wherein the method comprises administering a compound of Formula (I), Formula (II), Formula (III-a) or Formula (III-b), or a pharmaceutically acceptable salt, solvate, hydrate, tautomer, N-oxide, or stereoisomer thereof, or a composition thereof, to a subject.

In another aspect, the present invention features a method of treating a disease or disorder related to modulation (e.g., a decrease) in eIF2B activity or level, modulation (e.g., a decrease) of eIF2α activity or level, modulation (e.g., an increase) in eIF2α phosphorylation, modulation (e.g., an increase) of phosphorylated eIF2α pathway activity, or modulation (e.g., an increase) of ISR activity in a subject, wherein the method comprises administering a compound of Formula (I), Formula (II), Formula (III-a) or Formula (III-b), or a pharmaceutically acceptable salt, solvate, hydrate, tautomer, N-oxide or stereoisomer thereof, or a composition thereof, to a subject. In some embodiments, the disease may be caused by a mutation to a gene or protein sequence related to a member of the eIF2 pathway (e.g., the eIF2α signaling pathway or ISR pathway).

In another aspect, the present invention features a method of treating cancer in a subject, the method comprising administering to the subject a compound of Formula (I), Formula (II), Formula (III-a) or Formula (III-b) in combination with an immunotherapeutic agent.

### DETAILED DESCRIPTION OF THE INVENTION

The present invention features compounds, compositions, and methods comprising a compound of Formula (I), Formula (II), Formula (III-a) or Formula (III-b), or a pharmaceutically acceptable salt, solvate, hydrate, tautomer, N-oxide or stereoisomer thereof for use, e.g., in the modulation (e.g., activation) of eIF2B and the attenuation of the ISR signaling pathway.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions.

For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁-C₂₀ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₆ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. The term “alkenylene,” by itself or as part of another substituent, means, unless otherwise stated, a divalent radical derived from an alkene. An alkylene group may be described as, e.g., a C₁-C₆-membered alkylene, wherein the term “membered” refers to the non-hydrogen atoms within the moiety.

“Alkenyl” refers to a radical of a straight-chain or branched hydrocarbon group having from 2 to 20 carbon atoms, one or more carbon-carbon double bonds, and no triple bonds (“C₂-C₂₀ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g. phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

In certain embodiments, an aryl group is substituted with one or more of groups selected from halo, C₁-C₈ alkyl, halo-C₁-C₈ alkyl, haloxy-C₁-C₈ alkyl, cyano, hydroxy, alkoxy C₁-C₈ alkyl, and amino.

Examples of representative substituted aryls include the following

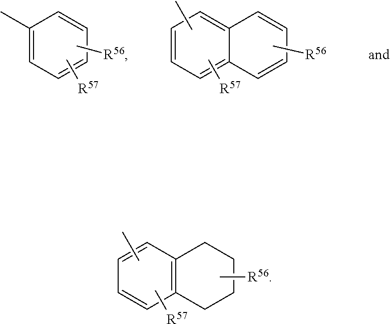

wherein one of R⁵⁶ and R⁵⁷ may be hydrogen and at least one of R⁵⁶ and R⁵⁷ is each independently selected from C₁-C₈ alkyl, halo-C₁-C₈ alkyl, 4-10 membered heterocyclyl, alkanoyl, alkoxy-C₁-C₈ alkyl, heteroaryloxy, alkylamino, arylamino, heteroarylamino, NR⁵⁸COR⁵⁹, NR⁵⁸SOR⁵⁹ NR⁵⁸SO₂R⁵⁹, C(O)Oalkyl, C(O)Oaryl, CONR⁵⁸R⁵⁹, CONR⁵⁸OR⁵⁹, NR⁵⁸R⁵⁹, SO₂NR⁵⁸R⁵⁹, S-alkyl, S(O)-alkyl, S(O)₂-alkyl, S-aryl, S(O)-aryl, S(O₂)-aryl; wherein R58 and R59 are independently hydrogen or C₁-C₈ alkyl; or R⁵⁶ and R⁵⁷ may be joined to form a cyclic ring (saturated or unsaturated) from 5 to 8 atoms, optionally containing one or more heteroatoms selected from the group N, O, or S.

Other representative aryl groups having a fused heterocyclyl group include the following:

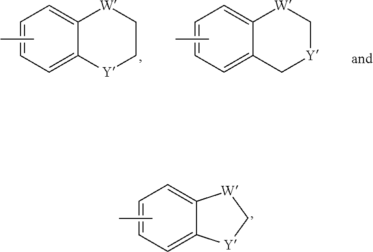

wherein each W′ is selected from C(R⁶⁶)₂, NR⁶⁶, O, and S; and each Y′ is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

Additionally, terms such as “haloalkyl” are meant to include monohaloalkyl and polyhaloalkyl. For example, the term “halo-C₁-C₆ alkyl” includes, but is not limited to, fluoromethyl, difluoromethyl, trifluoromethyl, 2,2,2-trifluoroethyl, 4-chlorobutyl, 3-bromopropyl, and the like.

The term “heteroalkyl,” by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)₂, —S(O)—CH₃, —S(O)₂—CH₃, —CH₂—CH₂—S(O)₂—CH₃, —CH═CH—O—CH₃, —Si(CH₃)₃, —CH₂—CH═N⇒OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{B}R^{C}, or the like, it will be understood that the terms heteroalkyl and —CH₂O or —NR^{B}R^{C} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{B}R^{C}, or the like.

Similarly, the term “heteroalkylene,” by itself or as part of another substituent, means, unless otherwise stated, a divalent radical derived from heteroalkyl, as exemplified, but not limited by, —CH₂O— and —CH₂CH₂O—. A heteroalkylene group may be described as, e.g., a 2-7-membered heteroalkylene, wherein the term “membered” refers to the non-hydrogen atoms within the moiety. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

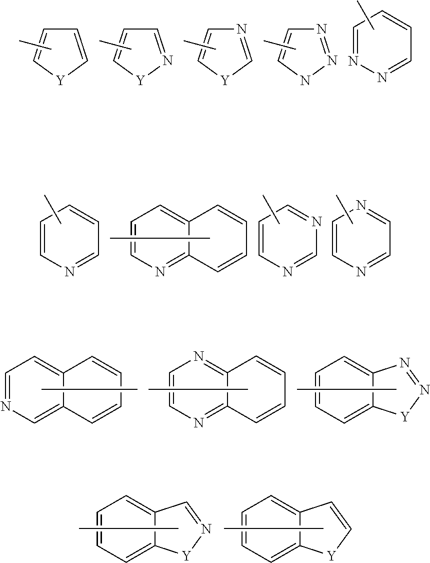

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

In some embodiments, “cycloalkyl” is a monocyclic, saturated cycloalkyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 5 to 6 ring carbon atoms (“C₁-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is substituted C₃-C₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

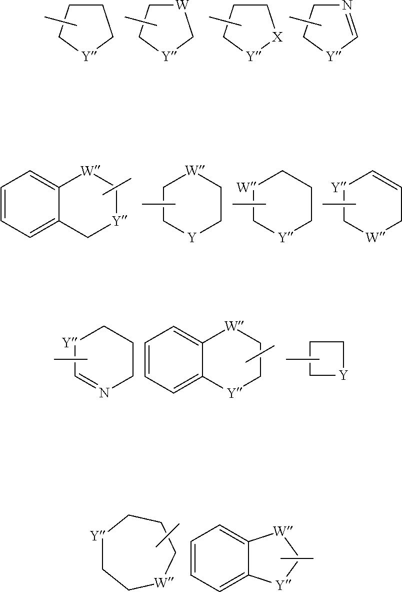

wherein each W″ is selected from CR⁶⁷, C(R⁶⁷)₂, NR⁶⁷, O, and S; and each Y″ is selected from NR⁶⁷, O, and S; and R⁷ is independently hydrogen, C₁-C₈ alkyl, C₃-C₁₀ cycloalkyl, 4-10 membered heterocyclyl, C₆-C₁₀ aryl, and 5-10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (e.g., amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Amino” refers to the radical —NR⁷⁰R⁷¹, wherein R⁷ and R⁷¹ are each independently hydrogen, C₁-C₈ alkyl, C₃-C₁₀ cycloalkyl, 4-10 membered heterocyclyl, C₆-C₁₀ aryl, and 5-10-membered heteroaryl. In some embodiments, amino refers to NH₂.

“Cyano” refers to the radical —CN.

“Hydroxy” refers to the radical —OH.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in a first buffer, e.g., in 1 mM-50 mM histidine, 0.1%-2% sucrose, 2%-7% mannitol at a pH range of 4.5 to 5.5, that is combined with a second buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer (e.g. pancreatic cancer, breast cancer, multiple myeloma, cancers of secretory cells), neurodegenerative diseases (e.g. Alzheimer's disease, Parkinson's disease, frontotemporal dementia), leukodystrophies (e.g., vanishing white matter disease, childhood ataxia with CNS hypo-myelination), postsurgical cognitive dysfunction, traumatic brain injury, stroke, spinal cord injury, intellectual disability syndromes, inflammatory diseases, musculoskeletal diseases, metabolic diseases, or diseases or disorders associated with impaired function of eIF2B or components in a signal transduction or signaling pathway including the ISR and decreased eIF2 pathway activity). For example certain methods herein treat cancer by decreasing or reducing or preventing the occurrence, growth, metastasis, or progression of cancer or decreasing a symptom of cancer; treat neurodegeneration by improving mental wellbeing, increasing mental function, slowing the decrease of mental function, decreasing dementia, delaying the onset of dementia, improving cognitive skills, decreasing the loss of cognitive skills, improving memory, decreasing the degradation of memory, decreasing a symptom of neurodegeneration or extending survival; treat vanishing white matter disease by reducing a symptom of vanishing white matter disease or reducing the loss of white matter or reducing the loss of myelin or increasing the amount of myelin or increasing the amount of white matter; treat childhood ataxia with CNS hypo-myelination by decreasing a symptom of childhood ataxia with CNS hypo-myelination or increasing the level of myelin or decreasing the loss of myelin; treat an intellectual disability syndrome by decreasing a symptom of an intellectual disability syndrome, treat an inflammatory disease by treating a symptom of the inflammatory disease; treat a musculoskeletal disease by treating a symptom of the musculoskeletal disease; or treat a metabolic disease by treating a symptom of the metabolic disease. Symptoms of a disease, disorder, or condition described herein (e.g., cancer, a neurodegenerative disease, a leukodystrophy, an inflammatory disease, a musculoskeletal disease, a metabolic disease, or a condition or disease associated with impaired function of eIF2B or components in a signal transduction pathway including the eIF2 pathway, eIF2α phosphorylation, or ISR pathway) would be known or may be determined by a person of ordinary skill in the art. The term “treating” and conjugations thereof, include prevention of an injury, pathology, condition, or disease (e.g. preventing the development of one or more symptoms of a disease, disorder, or condition described herein).

An “effective amount” is an amount sufficient to accomplish a stated purpose (e.g. achieve the effect for which it is administered, treat a disease, reduce enzyme activity, increase enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A “reduction” of a symptom or symptoms (and grammatical equivalents of this phrase) means decreasing of the severity or frequency of the symptom(s), or elimination of the symptom(s).

The term “associated” or “associated with” in the context of a substance or substance activity or function associated with a disease (e.g., a disease or disorder described herein, e.g., cancer, a neurodegenerative disease, a leukodystrophy, an inflammatory disease, a musculoskeletal disease, a metabolic disease, or a disease or disorder associated with impaired function of eIF2B or components in a signal transduction pathway including the eIF2 pathway, eIF2α phosphorylation, or ISR pathway) means that the disease is caused by (in whole or in part), or a symptom of the disease is caused by (in whole or in part) the substance or substance activity or function. For example, a symptom of a disease or condition associated with an impaired function of the eIF2B may be a symptom that results (entirely or partially) from a decrease in eIF2B activity (e.g. decrease in eIF2B activity or levels, increase in eIF2α phosphorylation or activity of phosphorylated eIF2α or reduced eIF2 activity or increase in activity of phosphorylated eIF2α signal transduction or the ISR signalling pathway). As used herein, what is described as being associated with a disease, if a causative agent, could be a target for treatment of the disease. For example, a disease associated with decreased eIF2 activity or eIF2 pathway activity, may be treated with an agent (e.g., compound as described herein) effective for increasing the level or activity of eIF2 or eIF2 pathway or a decrease in phosphorylated eIF2α activity or the ISR pathway. For example, a disease associated with phosphorylated eIF2α may be treated with an agent (e.g., compound as described herein) effective for decreasing the level of activity of phosphorylated eIF2α or a downstream component or effector of phosphorylated eIF2α. For example, a disease associated with eIF2α may be treated with an agent (e.g., compound as described herein) effective for increasing the level of activity of eIF2 or a downstream component or effector of eIF2.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. eIF2B, eIF2α, or a component of the eIF2 pathway or ISR pathway). In some embodiments contacting includes allowing a compound described herein to interact with a protein or enzyme that is involved in a signaling pathway (e.g. eIF2B, eIF2α, or a component of the eIF2 pathway or ISR pathway).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a signal transduction pathway or signaling pathway (e.g., eIF2B, eIF2α, or a component of the eIF2 pathway, pathway activated by eIF2α phosphorylation, or ISR pathway). Thus, inhibition may include, at least in part, partially or totally decreasing stimulation, decreasing or reducing activation, or inactivating, desensitizing, or down-regulating signal transduction or enzymatic activity or the amount of a protein increased in a disease (e.g. eIF2B, eIF2α, or a component of the eIF2 pathway or ISR pathway, wherein each is associated with cancer, a neurodegenerative disease, a leukodystrophy, an inflammatory disease, a musculoskeletal disease, or a metabolic disease). Inhibition may include, at least in part, partially or totally decreasing stimulation, decreasing or reducing activation, or deactivating, desensitizing, or down-regulating signal transduction or enzymatic activity or the amount of a protein (e.g. eIF2B, eIF2α, or component of the eIF2 pathway or ISR pathway) that may modulate the level of another protein or increase cell survival (e.g., decrease in phosphorylated eIF2α pathway activity may increase cell survival in cells that may or may not have an increase in phosphorylated eIF2α pathway activity relative to a non-disease control or decrease in eIF2α pathway activity may increase cell survival in cells that may or may not have an increase in eIF2α pathway activity relative to a non-disease control).

As defined herein, the term “activation”, “activate”, “activating” and the like in reference to a protein-activator (e.g. agonist) interaction means positively affecting (e.g. increasing) the activity or function of the protein (e.g. eIF2B, eIF2α, or component of the eIF2 pathway or ISR pathway) relative to the activity or function of the protein in the absence of the activator (e.g. compound described herein). In some embodiments, activation refers to an increase in the activity of a signal transduction pathway or signaling pathway (e.g. eIF2B, eIF2α, or component of the eIF2 pathway or ISR pathway). Thus, activation may include, at least in part, partially or totally increasing stimulation, increasing or enabling activation, or activating, sensitizing, or up-regulating signal transduction or enzymatic activity or the amount of a protein decreased in a disease (e.g. level of eIF2B, eIF2α, or component of the eIF2 pathway or ISR pathway associated with cancer, a neurodegenerative disease, a leukodystrophy, an inflammatory disease, a musculoskeletal disease, or a metabolic disease). Activation may include, at least in part, partially or totally increasing stimulation, increasing or enabling activation, or activating, sensitizing, or up-regulating signal transduction or enzymatic activity or the amount of a protein (e.g., eIF2B, eIF2α, or component of the eIF2 pathway or ISR pathway) that may modulate the level of another protein or increase cell survival (e.g., increase in eIF2α activity may increase cell survival in cells that may or may not have a reduction in eIF2α activity relative to a non-disease control).

The term “modulation” refers to an increase or decrease in the level of a target molecule or the function of a target molecule. In some embodiments, modulation of eIF2B, eIF2α, or a component of the eIF2 pathway or ISR pathway may result in reduction of the severity of one or more symptoms of a disease associated with eIF2B, eIF2α, or a component of the eIF2 pathway or ISR pathway (e.g., cancer, a neurodegenerative disease, a leukodystrophy, an inflammatory disease, a musculoskeletal disease, or a metabolic disease) or a disease that is not caused by eIF2B, eIF2α, or a component of the eIF2 pathway or ISR pathway but may benefit from modulation of eIF2B, eIF2α, or a component of the eIF2 pathway or ISR pathway (e.g., decreasing in level or level of activity of eIF2B, eIF2α or a component of the eIF2 pathway).

The term “modulator” as used herein refers to modulation of (e.g., an increase or decrease in) the level of a target molecule or the function of a target molecule. In embodiments, a modulator of eIF2B, eIF2α, or component of the eIF2 pathway or ISR pathway is an anti-cancer agent. In embodiments, a modulator of eIF2B, eIF2α, or component of the eIF2 pathway or ISR pathway is a neuroprotectant. In embodiments, a modulator of eIF2B, eIF2α, or component of the eIF2 pathway or ISR pathway is a memory enhancing agent. In embodiments, a modulator of eIF2B, eIF2α, or component of the eIF2 pathway or ISR pathway is a memory enhancing agent (e.g., a long term memory enhancing agent). In embodiments, a modulator of eIF2B, eIF2α, or component of the eIF2 pathway or ISR pathway is an anti-inflammatory agent. In some embodiments, a modulator of eIF2B, eIF2α, or component of the eIF2 pathway or ISR pathway is a pain-relieving agent.

“Patient” or “subject in need thereof refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

“Disease”, “disorder” or “condition” refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, e.g., through administration of a compound of Formula (I), Formula (II), Formula (III-a) or Formula (III-b) or a pharmaceutically acceptable salt thereof.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation).

The term “eIF2B” as used herein refers to the heteropentameric eukaryotic translation initiation factor 2B. eIF2B is composed of five subunits: eIF2B1, eIF2B2, eIF2B3, eIF2B4 and eIF2B5. eIF2B1 refers to the protein associated with Entrez gene 1967, OMIM 606686, Uniprot Q14232, and/or RefSeq (protein) NP_001405. eIF2B2 refers to the protein associated with Entrez gene 8892, OMIM 606454, Uniprot P49770, and/or RefSeq (protein) NP_055054. eIF2B3 refers to the protein associated with Entrez gene 8891, OMIM 606273, Uniprot Q9NR50, and/or RefSeq (protein) NP_065098. eIF2B4 refers to the protein associated with Entrez gene 8890, OMIM 606687, Uniprot Q9UI10, and/or RefSeq (protein) NP_751945. eIF2B5 refers to the protein associated with Entrez gene 8893, OMIM 603945, Uniprot Q13144, and/or RefSeq (protein) NP_003898.

The terms “eIF2alpha,” “eIF2α,” or “eIF2α” are interchangeable and refer to the protein “eukaryotic translation initiation factor 2 alpha subunit eIF2S1”. In embodiments, “eIF2alpha”, “eIF2α” or “eIF2α” refer to the human protein. Included in the terms “eIF2alpha”, “eIF2α” or “eIF2α” are the wild type and mutant forms of the protein. In embodiments, “eIF2alpha”, “eIF2α” or “eIF2α” refer to the protein associated with Entrez Gene 1965, OMIM 603907, UniProt P05198, and/or RefSeq (protein) NP_004085. In embodiments, the reference numbers immediately above refer to the protein and associated nucleic acids known as of the date of filing of this application.

### Compounds

Disclosed herein, for example, is a compound of Formula (I):

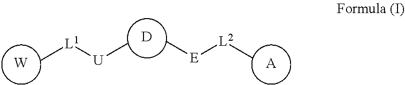

or a pharmaceutically acceptable salt, solvate, hydrate, tautomer, N-oxide, or stereoisomer thereof, wherein:

D is a bridged bicyclic cycloalkyl, a bridged bicyclic heterocyclyl, a 4-6-membered monocyclic cycloalkyl, a 4-6-membered monocyclic heterocyclyl, or cubanyl, wherein each bridged bicyclic cycloalkyl, bridged bicyclic heterocyclyl, 4-6-membered monocyclic cycloalkyl, 4-6-membered monocyclic heterocyclyl, or cubanyl is optionally substituted on one or more available carbons with 1-4 R^{X}; and wherein if the 4-6-membered monocyclic heterocyclyl or bridged bicyclic heterocyclyl contains a substitutable nitrogen moiety, the substitutable nitrogen may be optionally substituted by R^{N1};

U is —NR¹C(O)—, —C(O)NR¹— or 5-6-membered heteroaryl;

E is a bond, —NR²C(O)—, —C(O)NR²—, 5-6-membered heteroaryl or 5-6-membered heterocyclyl; wherein 5-6-membered heteroaryl or 5-6-membered heterocyclyl is optionally substituted on one or more available carbons with 1-5 R^{G}; and wherein if the 5-6-membered heteroaryl or 5-6-membered heterocyclyl contains a substitutable nitrogen moiety, the substitutable nitrogen may be optionally substituted by R^{N2}; or

E is

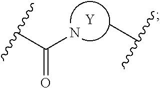

Y is a 4-9-membered nitrogen-containing monocyclic, bridged bicyclic, fused bicyclic or spirocyclic heterocyclyl, wherein the 4-9-membered nitrogen-containing monocyclic, bridged bicyclic, fused bicyclic or spirocyclic heterocyclyl is optionally substituted on one or more available carbons with 1-5 R^{G}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2};

L¹ is a bond, C₁-C₆ alkylene, 2-7 membered heteroalkylene, —NR^{N3}—, or —O—, wherein C₁-C₆ alkylene or 2-7 membered heteroalkylene is optionally substituted with 1-5 R^{L1}; L² is a bond, C₁-C₆ alkylene, 2-7 membered heteroalkylene, or —O—, wherein C₁-C₆ alkylene or 2-7 membered heteroalkylene is optionally substituted with 1-5 R^{L2};

R¹ is hydrogen or C₁-C₆ alkyl;

R² is hydrogen or C₁-C₆ alkyl;

W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available carbons with 1-4 R^{W1}; wherein the phenyl or heteroaryl may optionally be substituted on one or more available unsaturated carbons with 1-4 R^{W2}; wherein if the heterocyclyl contains a substitutable nitrogen moiety, the substitutable nitrogen may optionally be substituted with R^{N4} and wherein W is attached to L² through an available saturated carbon or nitrogen atom within the heterocyclyl;

A is C₃-C₆ cycloalkyl, phenyl, 4-6-membered heterocyclyl, 5-6-membered heteroaryl, or 8-10-membered bicyclic heteroaryl, wherein C₃-C₆ cycloalkyl, phenyl, 4-6-membered heterocyclyl, 5-6-membered heteroaryl, or 8-10-membered bicyclic heteroaryl is optionally substituted on one or more available carbons with 1-5 R^{Y}; and wherein if the 5-6-membered heteroaryl or 8-10-membered bicyclic heteroaryl contains a substitutable nitrogen moiety, the substitutable nitrogen may be optionally substituted by R^{N5};

each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N3} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

R^{N4} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B}R^{C} and —C(O)OR^{D};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O)₂—NR^{B}R^{C};

R^{N5} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};

each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A}, —NR^{B}R^{C}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or

2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};

each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};

each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, halo-C₁-C₆ alkoxy-C₁-C₆ alkyl, amino-C₁-C₆ alkyl, cyano-C₁-C₆ alkyl, halo, cyano, —OR^{A}, —NR^{B}R^{C}, —NR^{C}(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or

2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};

each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};

each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, and —S(O)₂R^{D};

R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};

each of R^{B} and R^{C} is independently hydrogen or C₁-C₆ alkyl;

R^{B} and R^{C} together with the atom to which they are attached form a 3-7-membered heterocyclyl ring optionally substituted with 1-3 R^{Z};

each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D} is independently C₁-C₆ alkyl or halo-C₁-C₆ alkyl;

each R^{E} is independently hydrogen, C₁-C₆ alkyl, or halo-C₁-C₆ alkyl;

each R^{F} is independently hydrogen, C₁-C₆ alkyl, or halo;

each R^{G} is independently hydrogen, C₁-C₆ alkyl, halo or oxo; and

m is 1 when R^{F} is hydrogen or C₁-C₆ alkyl, 3 when R^{F} is C₁-C₆ alkyl, or 5 when R^{F} is halo.

In some embodiments, D is bicyclo[1.1.1]pentane, bicyclo[2.2.1]heptane, bicyclo[2.1.1]hexane, bicyclo[2.2.2]octane, bicyclo[3.2.1]octane, 2-oxabicyclo[2.2.2]octane, 7-oxabicyclo[2.2.1]heptane, 8-azabicyclo[3.2.1]octane, cyclohexyl or tetrahydro-2H-pyranyl, each of which is optionally substituted with 1-4 R^{X} groups. In some embodiments, D is selected from the group consisting of

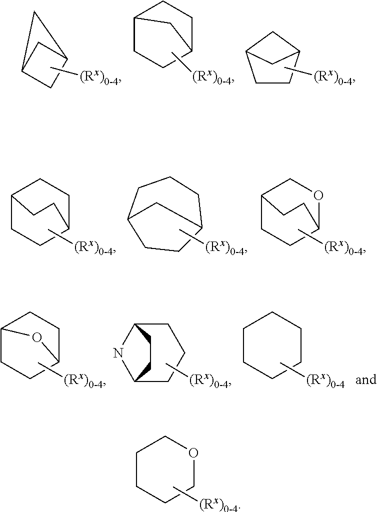

For example, in some embodiments D is selected from the group consisting of

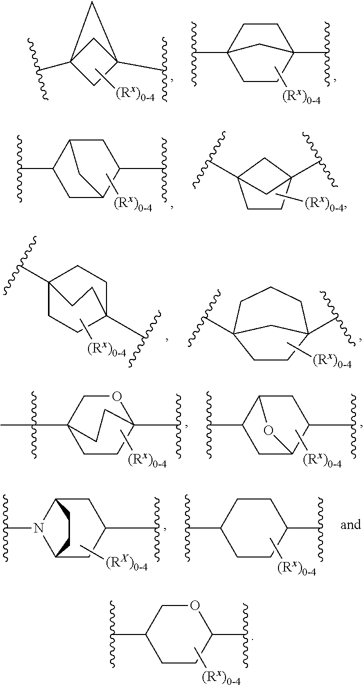

In some embodiments, D is substituted with 0 R^{X}. For example, in some embodiments D is selected from the group consisting of

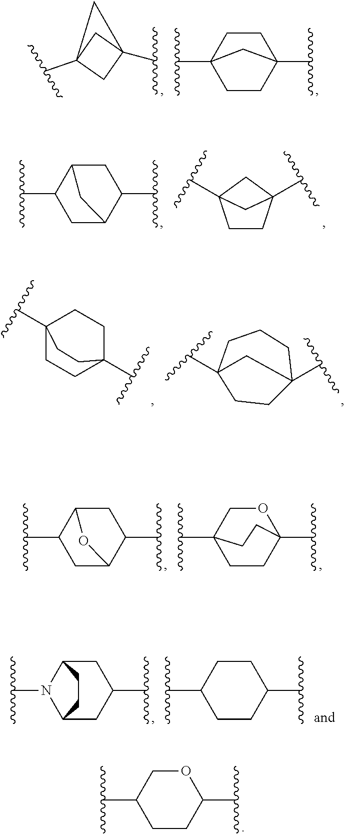

For example, in some embodiments D is

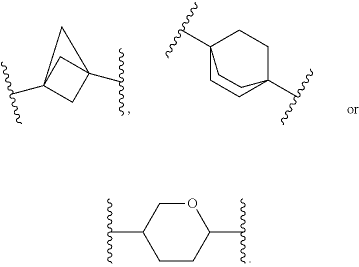 [SMILES: *C12CC(*)(C1)C2 ; *C12CCC(*)(CC1)CC2 ; *C1CCC(*)OC1]

In other embodiments, D is substituted with 1 R^{X}. For example, in some embodiments D is

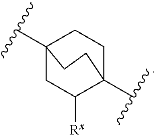

In certain embodiments, R^{X} is —OH.

In some embodiments, U is selected from the group consisting of —NHC(O)—, —C(O)NH— and

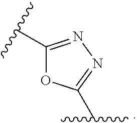 [SMILES: *c1nnc(*)o1]

For example, in certain embodiments U is —NHC(O)—.

In other embodiments, L¹ is a bond or C₁-C₆ alkylene, wherein C₁-C₆ alkylene is optionally substituted with 1-5 R^{L1}. For example, in some embodiments L¹ is a bond or C₁-C₆ alkylene, wherein C₁-C₆ alkylene is substituted with 0 R^{L1}. In certain embodiments, L¹ is, for example, a bond or —CH₂—. In certain other embodiments, R¹ is hydrogen or CH₃.

In further embodiments, W is represented by Formula (W-a):

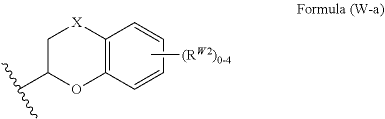

wherein:

X is NR^{N4} or C(R^{X1})(R^{X2});

R^{N4} is hydrogen or C₁-C₆ alkyl;

R^{X} is hydrogen or hydroxyl;

R^{X2} is hydrogen or hydroxyl; or

R^{X1} and R^{X2} taken together to form an oxo moiety.

For example, in some embodiments W is selected from the group consisting of

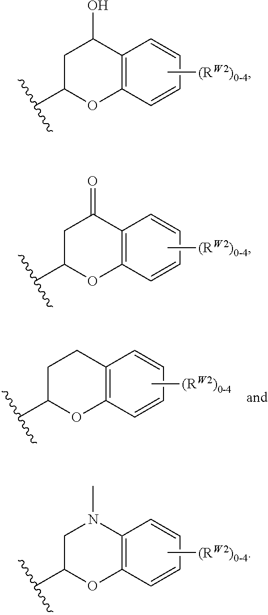

In certain embodiments W is, for example,

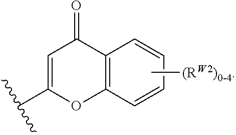

In some embodiments, W is substituted with 1 R^{W2}. For example, in certain embodiments R^{W2} is chloro. In other embodiments, W is substituted with 2 R^{W2}. For example, in certain embodiments each R^{W2} is independently chloro or fluoro.

In some embodiments, E is selected from the group consisting of a bond, —NR²C(O)—, —C(O)NR₂—, and

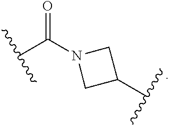 [SMILES: *C(=O)N1CC(*)C1]

In other embodiments, E is selected from the group consisting of

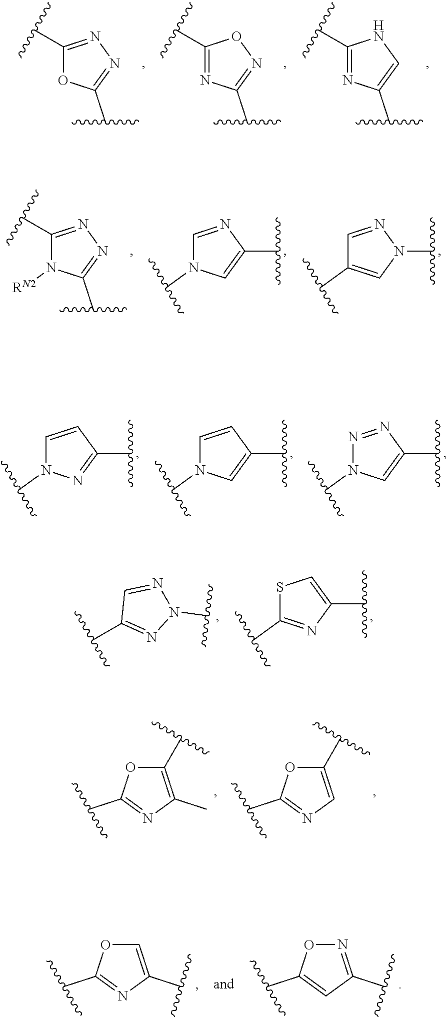

In further embodiments, E is selected from the group consisting of

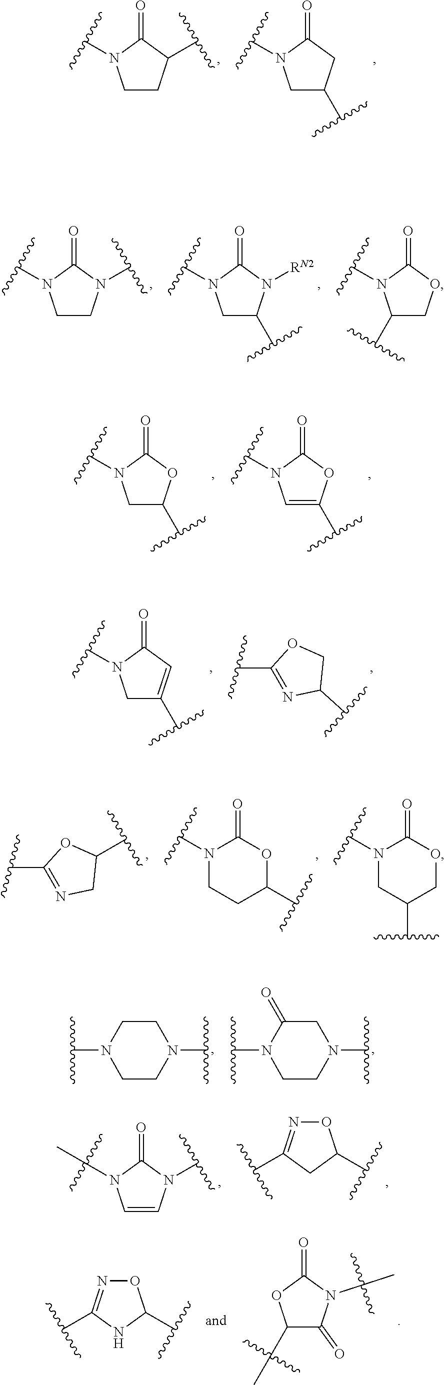

In yet further embodiments, E is selected from the group consisting of a bond, —NR₂C(O)—, —C(O)NR₂—,

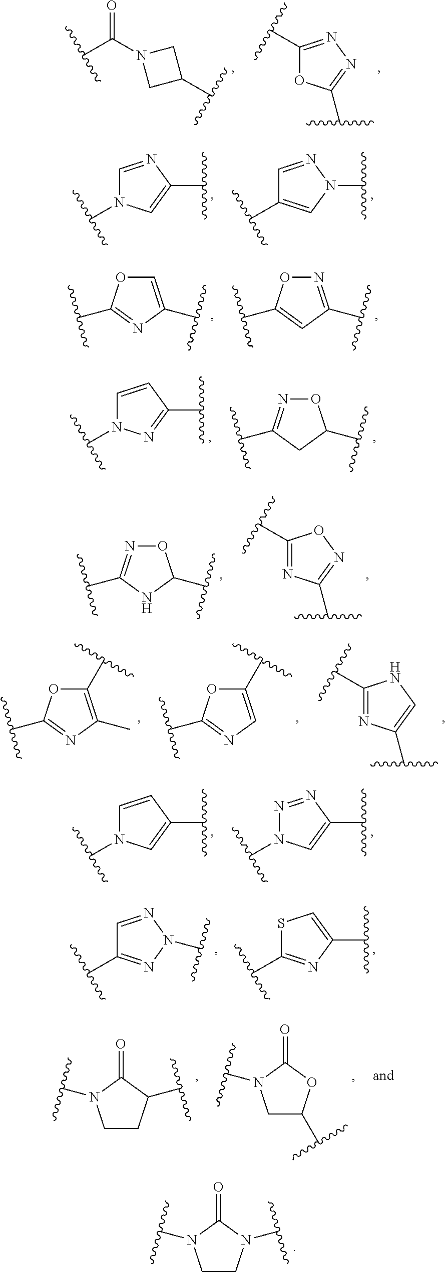

For example in certain embodiments E is selected from the group consisting of

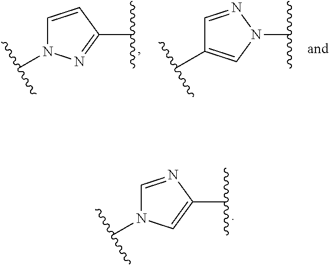 [SMILES: *c1ccn(*)n1 ; *c1cnn(*)c1 ; *c1cn(*)cn1]

In some embodiments, R² is hydrogen. In other embodiments, L² is a bond, —O—, C₁-C₆ alkylene, or 2-7 membered heteroalkylene. For example, in certain embodiments, L² is a bond, —CH₂—, —CH₂O—*, —(CH₂)₂O—*, (CH₂)₃O—*, or —O—, wherein “—*” indicates the attachment point to A.

In some embodiments, A is selected from the group consisting of:

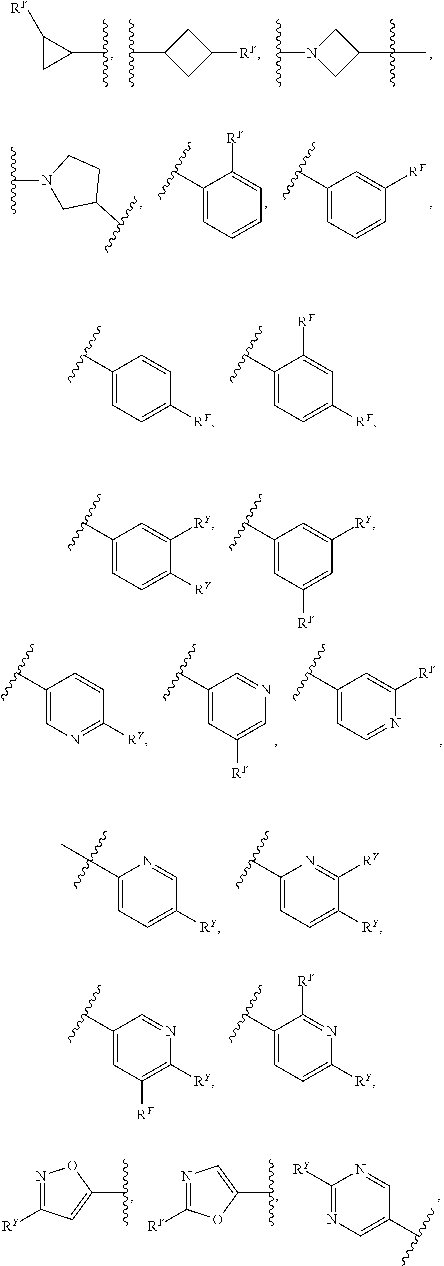

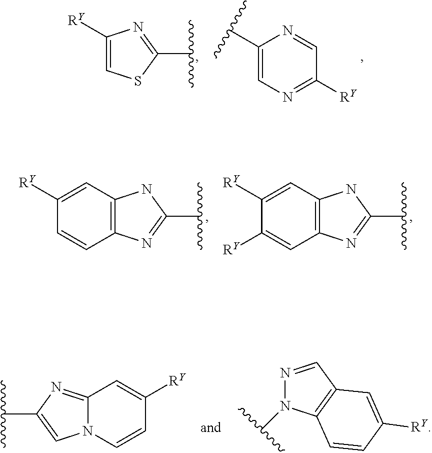

For example, in certain embodiments A is selected from the group consisting of:

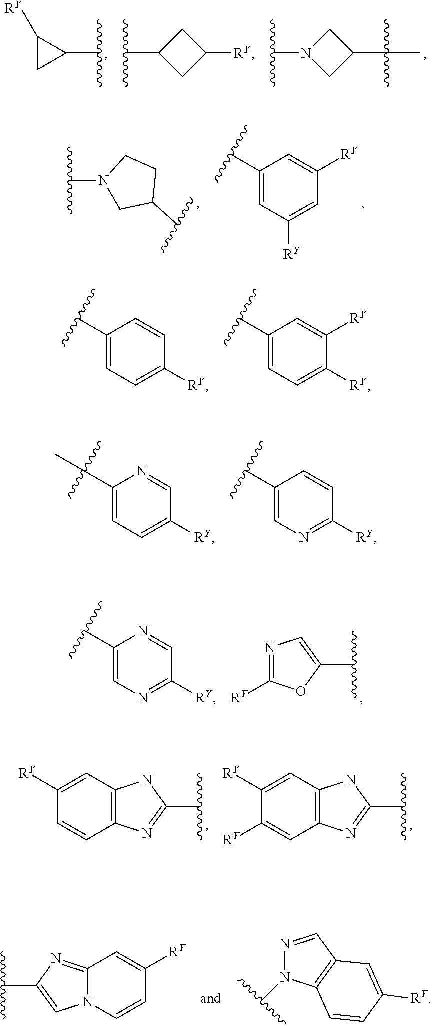

In some embodiments, each R^{Y} is independently selected from the group consisting of hydrogen, chloro, fluoro, hydroxyl, phenyl, CHF₂, CF₃, CH₃, CH₂CH₃, CH(CH₃)₂, OCH₃, OCHF₂, OCF₃, OCH₂CF₃, OCH(CH₃)₂, CH₂OCF₃, and CN.

Also disclosed herein is a compound of Formula (II):

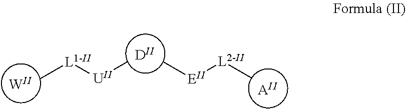

or a pharmaceutically acceptable salt, solvate, hydrate, tautomer, N-oxide, or stereoisomer thereof, wherein:

D^{II} is a bridged bicyclic cycloalkyl, a bridged bicyclic heterocyclyl, a 4-6-membered monocyclic cycloalkyl, a 4-6-membered monocyclic heterocyclyl, or cubanyl, wherein each bridged bicyclic cycloalkyl, bridged bicyclic heterocyclyl, 4-6-membered monocyclic cycloalkyl, 4-6-membered monocyclic heterocyclyl, or cubanyl is optionally substituted on one or more available carbons with 1-4 R^{X-II}; and wherein if the 4-6-membered monocyclic heterocyclyl or bridged bicyclic heterocyclyl contains a substitutable nitrogen moiety, the substitutable nitrogen may be optionally substituted by R^{N1-II};

U^{II} is —NR^{1-II}C(O)— or —C(O)NR^{1-II}—;

E^{II} is a bond, —NR^{2-II}C(O)—, —C(O)NR^{2-II}—, 5-6-membered heteroaryl or 5-6-membered heterocyclyl; wherein 5-6-membered heteroaryl or 5-6-membered heterocyclyl is optionally substituted on one or more available carbons with 1-5 R^{G-II}; and wherein if the 5-6-membered heteroaryl or 5-6-membered heterocyclyl contains a substitutable nitrogen moiety, the substitutable nitrogen may be optionally substituted by R^{N2-II}; or

E^{II} is

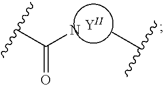

Y^{II} is a 4-9-membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{G-II}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2-II};

L^{1-II} is a bond, C₁-C₆ alkylene, 2-7 membered heteroalkylene, —NR^{N3-II}, or —O—, wherein C₁-C₆ alkylene or 2-7 membered heteroalkylene is optionally substituted with 1-5 R^{L1-II};

L^{2-II} is a bond, C₁-C₆ alkylene, or 2-7 membered heteroalkylene, —O—, wherein C₁-C₆ alkylene or 2-7 membered heteroalkylene is optionally substituted with 1-5 R^{L2-II};

R^{1-II} is hydrogen or C₁-C₆ alkyl;

R^{2-II} is hydrogen or C₁-C₆ alkyl;

W^{II} is phenyl or 5-6-membered heteroaryl; wherein phenyl or 5-6-membered heteroaryl is optionally substituted with 1-5 R^{W-II}; and wherein if the 5-6-membered heteroaryl contains a substitutable nitrogen moiety, the substitutable nitrogen may be optionally substituted by R^{N4-II};

A^{II} is C₃-C₆ cycloalkyl, phenyl, or 5-6-membered heteroaryl, wherein C₃-C₆ cycloalkyl, phenyl, or 5-6-membered heteroaryl is optionally substituted on one or more available carbons with 1-5 R^{Y-II}; and wherein if the 5-6-membered heteroaryl contains a substitutable nitrogen moiety, the substitutable nitrogen may be optionally substituted by R^{N5-II};

each R^{L1-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};

each R^{L2-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};

R^{N1-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N2-II} is selected from the group consisting of hydrogen. C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N3-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

R^{N4-II} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B-II}R^{C-II} and C(O)OR^{D-II};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w-II}C₁₋₆ alkyl (wherein w-II is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O₂)NR^{B-II}R^{C-II};

R^{N5-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};

each R^{W-II} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, oxo, C═N—OH, halo, cyano, —OR^{A-II}, —NR^{B-II}R^{C-II}, —NR^{B-II}R^{CC-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II}; or

2 R^{W-II} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-II};

each R^{X-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};

each R^{Y-II} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —S(R^{F-II})_{m-II}, —S(O)R^{D-II}, —S(O)₂R^{D-II}, and G^{1-II}; or

2 R^{Y-II} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-II};

each G^{1-II} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-II};

each R^{Z-II} is independently selected from the group consisting of C₁-C₆ alkyl, hydroxy-C₁-C₆ alkyl, halo-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II} and —S(O)₂R^{D-II};

R^{A-II} is, at each occurrence, independently hydrogen, C₁-C₆ alkyl, halo-C₁-C₆ alkyl, —C(O)NR^{B-II}R^{C-II}, —C(O)R^{D-II} or —C(O)OR^{D-II};

each of R^{B-II} and R^{C-II} is independently hydrogen or C₁-C₆ alkyl;

R^{B-II} and R^{C-II} together with the atom to which they are attached form a 3-7-membered heterocyclyl ring optionally substituted with 1-3 R^{Z-II};

each R^{CC-II} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D-II} is independently C₁-C₆ alkyl or halo-C₁-C₆ alkyl;

each R^{E-II} is independently hydrogen, C₁-C₆ alkyl, or halo-C₁-C₆ alkyl;

each R^{F-II} is independently hydrogen, C₁-C₆ alkyl, or halo; and

each R^{G-II} is independently hydrogen, C₁-C₆ alkyl, halo or oxo;

provided that when D^{II} is a bridged bicyclic 5-membered cycloalkyl, E^{II} is —NR^{2-II}C(O)—.

In some embodiments, D^{II} is bicyclo[1.1.1]pentane, bicyclo[2.2.1]heptane, bicyclo[2.1.1]hexane, bicyclo[2.2.2]octane, bicyclo[3.2.1]octane, 7-oxabicyclo[2.2.1]heptane, 8-azabicyclo[3.2.1]octane, cyclohexyl or tetrahydro-2H-pyranyl, each of which is optionally substituted with 1-4 R^{X-II} groups.

For example, in some embodiments D^{II} is selected from the group consisting of

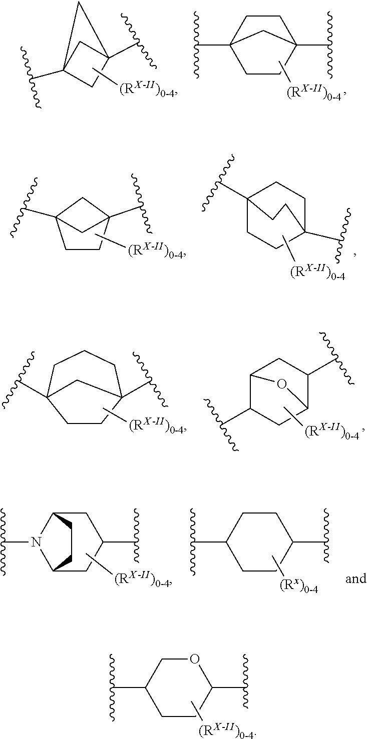

In some embodiments, D^{II} is substituted with 0 R^{X-II}. For example, in some embodiments D^{II} is selected from the group consisting of

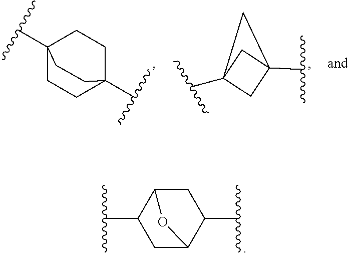 [SMILES: *C12CCC(*)(CC1)CC2 ; *C12CC(*)(C1)C2 ; *C1CC2OC1CC2*]

In other embodiments, D^{II} is substituted with 1 R^{X-II}. For example, in certain embodiment D^{II} is

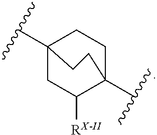

In some embodiments, R^{X-II} is —OH.

In further embodiments, L^{1-II} is a C₁-C₆ alkylene or a 2-7 membered heteroalkylene, wherein the C₁-C₆ alkylene or 2-7 membered heteroalkylene is optionally substituted with 1-5 R^{L1-II}. For example, in some embodiments L^{1-II} is a C₁-C₆ alkylene or a 2-7 membered heteroalkylene substituted with 0 R^{L1-II}. In certain embodiments, for example, L^{1-II} is —CH₂— or CH₂O—*, wherein “—*” indicates the attachment point to W^{II}.

In some embodiments, R^{1-II} is hydrogen or CH₃. In other embodiments, W^{II} is selected from the group consisting of

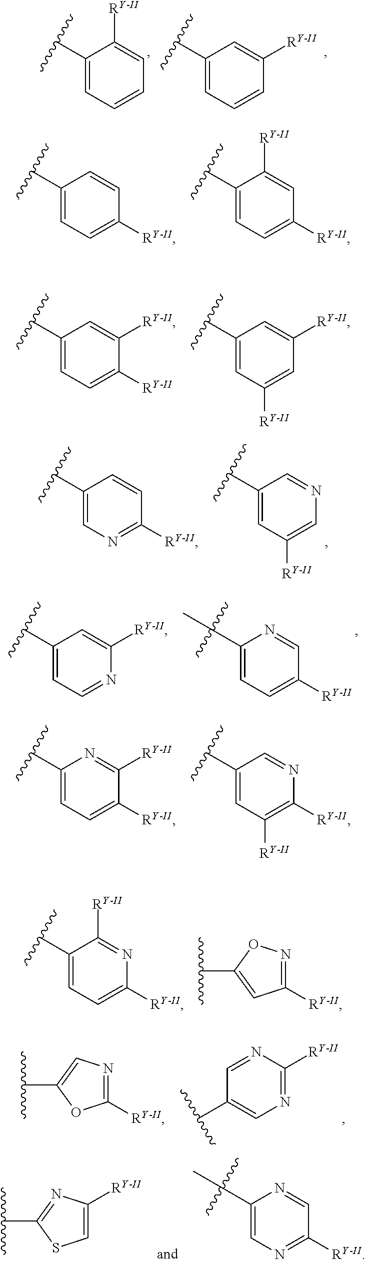

For example, in certain embodiments W^{II} is

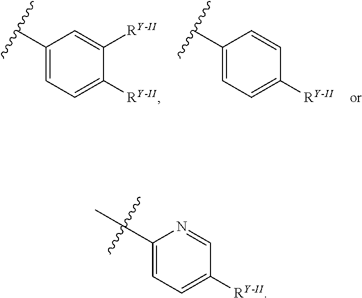

In other embodiments, R^{Y-II} is independently chloro, fluoro or CF₃.

In some embodiments, E^{II} is selected from the group consisting of NR^{2-II}C(O)—, —C(O)R^{2-II}—, and

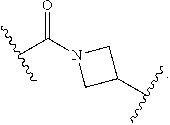 [SMILES: *C(=O)N1CC(*)C1]

In other embodiments, E^{II} is selected from the group consisting of

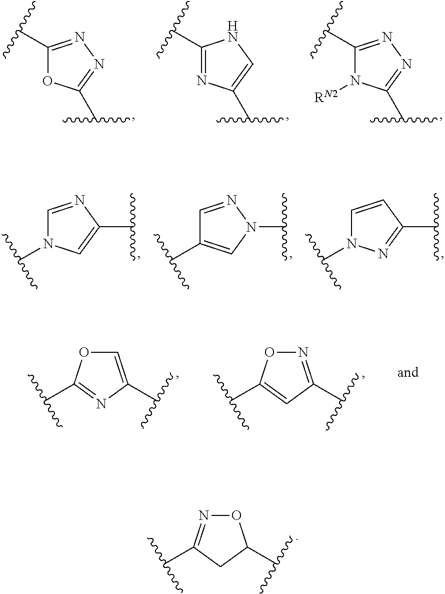

For example, in certain embodiments E^{II} is selected from the group consisting of —NR^{2-II}C(O)—,

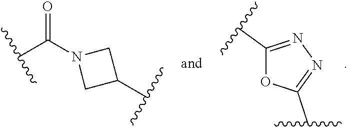 [SMILES: *C(=O)N1CC(*)C1 ; *c1nnc(*)o1]

In further embodiments, E^{II} is —NR^{2-II}C(O)— when D^{II} is

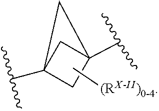

In some embodiments, R^{2-II} is hydrogen or methyl. In other embodiments, L^{2-II} is a bond, —O—, or 2-7 membered heteroalkylene. For example, in certain embodiments L^{2-II} is a bond, —CH₂O—*, —(CH₂)₂O—*, —(CH₂)₃O—*, or —O—, wherein “—*” indicates the attachment point to A^{II}.

In some embodiments, A^{II} is selected from the group consisting of:

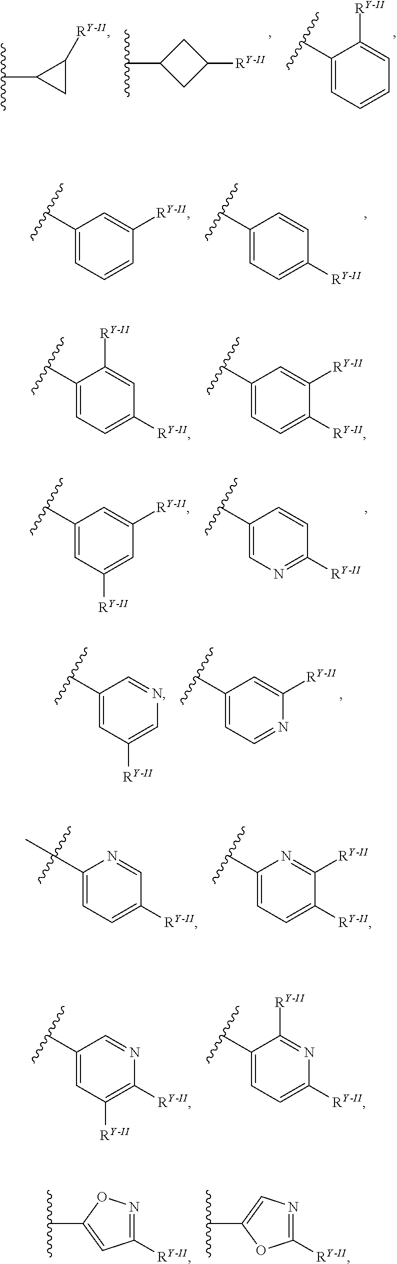

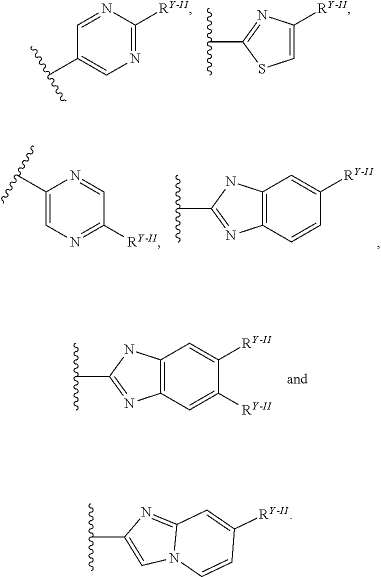

For example, in certain embodiments A^{II} is

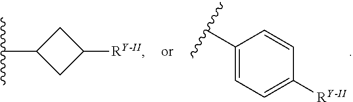

In other embodiments, each R^{Y-II} is chloro or OCF₃.

Also disclosed is a compound represented by Formula (IIIa) or Formula (IIIb):

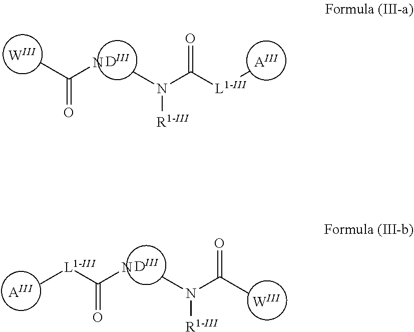

or a pharmaceutically acceptable salt, solvate, hydrate, tautomer, N-oxide, or stereoisomer thereof, wherein:

D^{III} is a 4-9 membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{X-III}; and wherein if the 4-9 membered nitrogen-containing monocyclic, bridged bicyclic, fused bicyclic or spirocyclic heterocyclyl contains a substitutable nitrogen moiety, the substitutable nitrogen may be optionally substituted by R^{N1-III};

W^{III} is a 8-10 membered, partially unsaturated, fused bicyclic ring moiety comprising a 5-6 membered heterocyclyl fused to a phenyl or 5-6-membered heteroaryl, wherein the heterocyclyl may be optionally substituted on one or more available saturated carbons with 1-4 R^{W1-III}; wherein the phenyl or heteroaryl may optionally be substituted on one or more available unsaturated carbons with 1-4 R^{W2-III}; and wherein if the heterocyclyl contains a substitutable nitrogen moiety, the substitutable nitrogen may optionally be substituted with R^{N2-III};

A^{III} is phenyl or 5-6-membered heteroaryl, wherein phenyl or 5-6-membered heteroaryl is optionally substituted on one or more available carbons with 1-5 R^{Y-III}; and wherein if the 5-6-membered heteroaryl contains a substitutable nitrogen moiety, the substitutable nitrogen may be optionally substituted by R^{N3-III};

R^{1-III} is hydrogen or C₁-C₆ alkyl;

L^{1-III} is a bond, C₁-C₆ alkylene or 2-7 membered heteroalkylene, wherein C₁-C₆ alkylene or 2-7 membered heteroalkylene is optionally substituted with 1-5 R^{L1-III};

each R^{L1-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

R^{N1-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};

R^{N2-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{X-III}, —C(O)R^{D-III}, —C(O)OR^{D-III}, and —S(O)₂R^{D-III};

R^{N3-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};

each R^{W1-III} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A-III}, —NR^{B-III}R^{C-III}, —NR^{B-III}R^{CC-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

each R^{W2-III} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —S(R^{F-II})ₘ-ml, —S(O)R^{D-II}, and —S(O)₂R^{D-III}; or

2 R^{W2-III} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-III};

each R^{X-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};

each R^{Y-III} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —S(R^{F-III})_{m-III}, —S(O)R^{D-III}, —S(O)₂R^{D-III}, and G^{1-III}; or

2 R^{Y-III} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-III};

each G^{1-III} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-III};

each R^{Z-III} is independently selected from the group consisting of C₁-C₆ alkyl, hydroxy-C₁-C₆ alkyl, halo-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, and —S(O)₂R^{D-III};

R^{A-III} is, at each occurrence, independently hydrogen, C₁-C₆ alkyl, halo-C₁-C₆ alkyl, —C(O)NR^{B-III}R^{C-III}, —C(O)R^{D-III}, or —C(O)OR^{D-III};

each of R^{B-III} and R^{C-III} is independently hydrogen or C₁-C₆ alkyl; or

R^{B-III} and R^{C-III} together with the atom to which they are attached form a 3-7-membered heterocyclyl ring optionally substituted with 1-3 R^{Z-III};

each R^{CC-III} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;

each R^{D-III} is independently C₁-C₆ alkyl, hydroxy-C₁-C₆ alkyl, or halo-C₁-C₆ alkyl;

each R^{E-III} is independently hydrogen, C₁-C₆ alkyl, or halo-C₁-C₆ alkyl;

each R^{F-III} is independently hydrogen, C₁-C₆ alkyl, or halo; and

m^{III} is 1 when R^{F-III} is hydrogen or C₁-C₆ alkyl, 3 when R^{F-III} is C₁-C₆ alkyl, or 5 when R^{F-III} is halo.

In some embodiments, D^{III} is an azetidine, pyrrolidine, piperidine, piperazine, or 2-azaspiro[3.3]heptane moiety, each of which is optionally substituted with 1-4 R^{W-III} groups, and each R^{W-III} is independently C₁-C₆ alkyl, halo-C₁-C₆ alkyl, halo, oxo, cyano, or —OR^{A-III}, and wherein piperazine is optionally substituted on a substitutable nitrogen by R^{N2-III}.

For example, in some embodiments D^{III} is selected from the group consisting of:

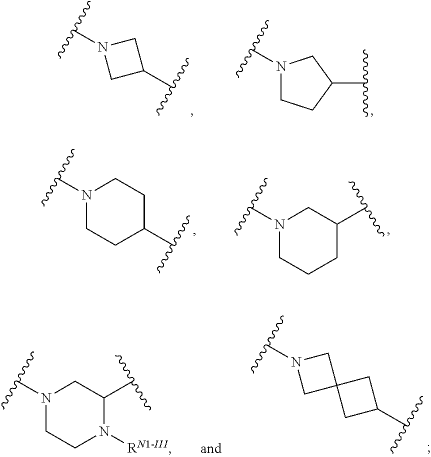

wherein R^{N1-III} is hydrogen or C₁-C₃ alkyl. For example, in certain embodiments D^{III} is

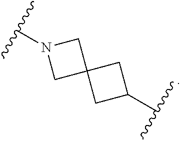 [SMILES: *C1CC2(C1)CN(*)C2]

In some embodiments, W^{III} is represented by Formula (W-b):

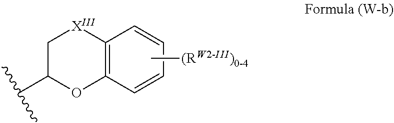

wherein:

X^{III} is NR^{N4-III} or C(R^{X1-III})(R^{X2-III});

R^{N4-III} is hydrogen or C₁-C₆ alkyl;

R^{X1-III} is hydrogen or hydroxyl;

R^{X2-III} is hydrogen or hydroxyl; or

R^{X1-III} and R^{X2-III} taken together to form an oxo moiety.

For example, in some embodiments W^{III} is selected from the group consisting of

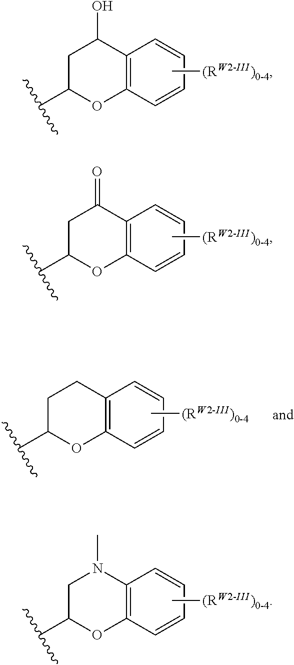

In some embodiments, W^{III} is substituted with 1 R^{W2-III}. For example, in certain embodiments R^{W2-III} is chloro.

In some embodiments, L^{1-III} is 2-7 membered heteroalkylene optionally substituted by 1-5 R^{L1-III}. In other embodiment, L^{1-III} is 2-7 membered heteroalkylene substituted by 0 R^{L1}. For example, in certain embodiments L^{1-III} is selected from CH₂O—* or CH₂OCH₂—*, wherein “—*” indicates the attachment point to A^{III}. In other embodiments, R^{1-III} is hydrogen or CH₃.

In some embodiments, A^{III} is selected from the group consisting of:

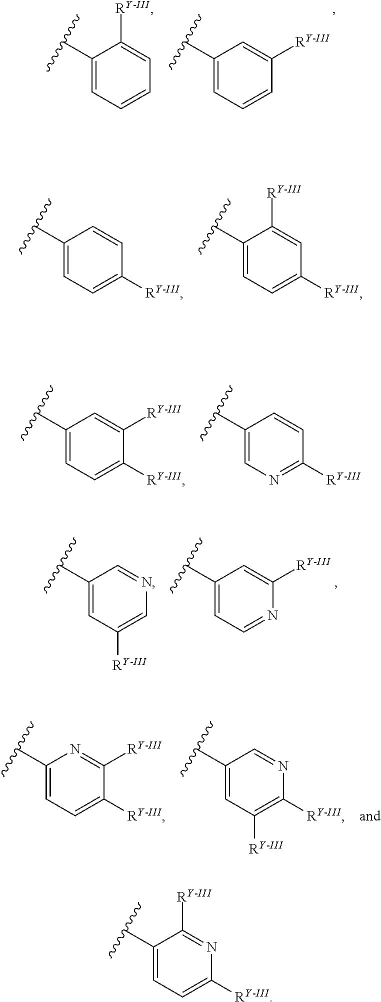

In some embodiments, each R^{Y}n is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN.

In some embodiments, a disclosed compound is selected from the group consisting of
(2R)-6-chloro-N-(3-{5-[(3,5-dimethylphenoxy)methyl]-2-oxo-1,3-oxazolidin-3-yl}bicyclo[1.1.1]pentan-1-yl)-4-oxo-3,4-dihydro-2H-1-benzopyran-2-carboxamide;
(2R)-6-chloro-N-{(1R,3r,5S)-8-[3-(4-chlorophenoxy)propyl]-8-azabicyclo[3.2.1]octan-3-yl}-4-oxo-3,4-dihydro-2H-1-benzopyran-2-carboxamide;
(2R,4R)-6-chloro-N-[(1r,4R)-4-{[(4-chloro-3-fluorophenoxy)acetyl](methyl)amino}cyclohexyl]-4-hydroxy-3,4-dihydro-2H-1-benzopyran-2-carboxamide;
3-[2-(4-chloro-3-fluorophenoxy)acetamido]-N-[(6-chloro-4-oxo-3,4-dihydro-2H-1-benzopyran-2-yl)methyl]bicyclo[1.1.1]pentane-1-carboxamide;
3-[2-(4-chloro-3-fluorophenoxy)acetamido]-N-[(6-chloro-4-hydroxy-3,4-dihydro-2H-1-benzopyran-2-yl)methyl]bicyclo[1.1.1]pentane-1-carboxamide;
(2R,4R)-6-chloro-4-hydroxy-N-[(1r,4R)-4-({[5-(trifluoromethyl)pyridin-2-yl]methyl}carbamoyl)cyclohexyl]-3,4-dihydro-2H-1-benzopyran-2-carboxamide;
(2R)-6-chloro-4-oxo-N-[4-({[5-(trifluoromethyl)pyridin-2-yl]methyl}carbamoyl)bicyclo[2.2.2]octan-1-yl]-3,4-dihydro-2H-1-benzopyran-2-carboxamide;
(2R,4R)-6-chloro-4-hydroxy-N-[4-({[5-(trifluoromethyl)pyridin-2-yl]methyl}carbamoyl)bicyclo[2.2.2]octan-1-yl]-3,4-dihydro-2H-1-benzopyran-2-carboxamide;
(2R)-6-chloro-N-(3-{5-[(4-chloro-3-fluorophenoxy)methyl]-1,3,4-oxadiazol-2-yl}bicyclo[1.1.1]pentan-1-yl)-4-oxo-3,4-dihydro-2H-1-benzopyran-2-carboxamide;
(2S)-6-chloro-N-(3-{5-[(4-chloro-3-fluorophenoxy)methyl]-1,3,4-oxadiazol-2-yl}bicyclo[1.1.1]pentan-1-yl)-4-oxo-3,4-dihydro-2H-1-benzopyran-2-carboxamide;
(2R,4R)-6-chloro-N-(3-{5-[(4-chloro-3-fluorophenoxy)methyl]-1,3,4-oxadiazol-2-yl}bicyclo[1.1.1]pentan-1-yl)-4-hydroxy-3,4-dihydro-2H-1-benzopyran-2-carboxamide;
(2S,4S)-6-chloro-N-(3-{5-[(4-chloro-3-fluorophenoxy)methyl]-1,3,4-oxadiazol-2-yl}bicyclo[1.1.1]pentan-1-yl)-4-hydroxy-3,4-dihydro-2H-1-benzopyran-2-carboxamide;
2-(4-chloro-3-fluorophenoxy)-N-[(2S)-2-hydroxy-4-(2-{[(1s,3R)-3-(trifluoromethoxy)cyclobutyl]oxy}acetamido)bicyclo[2.2.2]octan-1-yl]acetamide;
6-chloro-4-oxo-N-[3-(2-{[(1s,3s)-3-(trifluoromethoxy)cyclobutyl]oxy}acetamido)bicyclo[1.1.1]pentan-1-yl]-3,4-dihydro-2H-1-benzopyran-2-carboxamide;
6-chloro-4-hydroxy-N-[3-(2-{[(1s,3s)-3-(trifluoromethoxy)cyclobutyl]oxy}acetamido)bicyclo[1.1.1]pentan-1-yl]-3,4-dihydro-2H-1-benzopyran-2-carboxamide;
(2R)-6-chloro-N-[(3S)-3-hydroxy-4-(2-{[(1s,3R)-3-(trifluoromethoxy)cyclobutyl]oxy}acetamido)bicyclo[2.2.2]octan-1-yl]-4-oxo-3,4-dihydro-2H-1-benzopyran-2-carboxamide;
2-(4-chlorophenoxy)-N-[4-(2-{[(1s,3s)-3-(trifluoromethoxy)cyclobutyl]oxy}acetamido)bicyclo[2.2.2]octan-1-yl]acetamide;
(2R,4R)-6-chloro-4-hydroxy-N-[(3S)-3-hydroxy-4-(2-{[(1s,3R)-3-(trifluoromethoxy)cyclobutyl]oxy}acetamido)bicyclo[2.2.2]octan-1-yl]-3,4-dihydro-2H-1-benzopyran-2-carboxamide;
(1s,3s)-N-{3-[2-(4-chloro-3-fluorophenoxy)acetamido]bicyclo[1.1.1]pentan-1-yl}-3-(trifluoromethoxy)cyclobutane-1-carboxamide;
(2R,4R)-6-chloro-4-hydroxy-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide;
2-(4-chloro-3-fluorophenoxy)-N-{rac-(3R,6S)-6-[3-(4-chlorophenoxy)azetidine-1-carbonyl]oxan-3-yl}acetamide;
6-chloro-4-hydroxy-N-[rac-(3R,6S)-6-({[4-(trifluoromethyl)phenyl]methyl}carbamoyl)oxan-3-yl]-3,4-dihydro-2H-1-benzopyran-2-carboxamide;
6-chloro-N-{rac-(3R,6S)-6-[3-(4-chlorophenoxy)azetidine-1-carbonyl]oxan-3-yl}-4-hydroxy-3,4-dihydro-2H-1-benzopyran-2-carboxamide;
rac-(2R,4R)-6-chloro-4-hydroxy-N-[3-(([5-(trifluoromethyl)pyridin-2-yl]methyl)carbamoyl)bicyclo[1.1.1]pentan-1-yl]-3,4-dihydro-2H-1-benzopyran-2-carboxamide;
2-(4-chloro-3-fluorophenoxy)-N-(2-hydroxy-4-{5-[(1s,3s)-3-(trifluoromethoxy)cyclobutyl]-1,3,4-oxadiazol-2-yl}bicyclo[2.2.2]octan-1-yl)acetamide;
(2R,4R)-6-chloro-N-{(1R,3r,5S)-8-[3-(4-chlorophenoxy)propyl]-8-azabicyclo[3.2.1]octan-3-yl}-4-hydroxy-3,4-dihydro-2H-1-benzopyran-2-carboxamide;
6-chloro-N-[(1r,4r)-4-{[(6-chloro-1H-benzimidazol-2-yl)methyl]carbamoyl}cyclohexyl]-4-hydroxy-3,4-dihydro-2H-1-benzopyran-2-carboxamide;
(2R,4R)-6-chloro-N-(3-{[(5,6-difluoro-1H-benzimidazol-2-yl)methyl]carbamoyl}bicyclo[1.1.1]pentan-1-yl)-4-hydroxy-3,4-dihydro-2H-1-benzopyran-2-carboxamide;
(2R,4R)-6-chloro-4-hydroxy-N-(3-{[(1s,3S)-3-(trifluoromethoxy)cyclobutane-1-carbonyl]amino}bicyclo[1.1.1]pentan-1-yl)-3,4-dihydro-2H-1-benzopyran-2-carboxamide;
N-[(6-chloro-3,4-dihydro-2H-1-benzopyran-2-yl)methyl]-3-[2-(4-chloro-3-fluorophenoxy)acetamido]bicyclo[1.1.1]pentane-1-carboxamide;
6-chloro-N-{(1r,4r)-4-[2-(4-chloro-3-fluorophenoxy)acetamido]cyclohexyl}-4-oxo-3,4-dihydro-2H-1-benzopyran-2-carboxamide;
6-chloro-N-[rac-(3R,6S)-6-{[(7-chloroimidazo[1,2-a]pyridin-2-yl)methyl]carbamoyl}oxan-3-yl]-4-hydroxy-3,4-dihydro-2H-1-benzopyran-2-carboxamide;
(2R)-6-chloro-4-oxo-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide;
(2R)-6-chloro-4-oxo-N-(3-{[(1s,3S)-3-(trifluoromethoxy)cyclobutane-1-carbonyl]amino}bicyclo[1.1.1]pentan-1-yl)-3,4-dihydro-2H-1-benzopyran-2-carboxamide;
6-chloro-4-oxo-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide;
(2R)-6-chloro-N-(3-{[(5,6-difluoro-1H-benzimidazol-2-yl)methyl]carbamoyl}bicyclo[1.1.1]pentan-1-yl)-4-oxo-3,4-dihydro-2H-1-benzopyran-2-carboxamide;
6-chloro-N-[(1r,4r)-4-{3-[5-(difluoromethyl)pyrazin-2-yl]-2-oxoimidazolidin-1-yl}cyclohexyl]-4-hydroxy-3,4-dihydro-2H-1-benzopyran-2-carboxamide;
6-chloro-4-oxo-N-[rac-(3R,6S)-6-({[4-(trifluoromethyl)phenyl]methyl}carbamoyl)oxan-3-yl]-3,4-dihydro-2H-1-benzopyran-2-carboxamide;
6-chloro-N-[(1r,4r)-4-{[(6-chloro-1H-benzimidazol-2-yl)methyl]carbamoyl}cyclohexyl]-4-oxo-3,4-dihydro-2H-1-benzopyran-2-carboxamide;
6-chloro-N-{rac-(3R,6S)-6-[3-(4-chlorophenoxy)azetidine-1-carbonyl]oxan-3-yl}-4-oxo-3,4-dihydro-2H-1-benzopyran-2-carboxamide;
6-chloro-N-[rac-(3R,6S)-6-{[(7-chloroimidazo[1,2-a]pyridin-2-yl)methyl]carbamoyl}oxan-3-yl]-4-oxo-3,4-dihydro-2H-1-benzopyran-2-carboxamide;
6-chloro-N-{(1r,4r)-4-[2-(4-chloro-3-fluorophenoxy)acetamido]cyclohexyl}-4-hydroxy-3,4-dihydro-2H-1-benzopyran-2-carboxamide;
(2R,4R)-6-chloro-N-(3-{5-[(3,5-dimethylphenoxy)methyl]-2-oxo-1,3-oxazolidin-3-yl}bicyclo[1.1.1]pentan-1-yl)-4-hydroxy-3,4-dihydro-2H-1-benzopyran-2-carboxamide;
(2R,4R)-6-chloro-N-{2-[(4-chloro-3-fluorophenoxy)acetyl]-2-azaspiro[3.3]heptan-6-yl}-4-hydroxy-3,4-dihydro-2H-1-benzopyran-2-carboxamide;
2-(4-chloro-3-fluorophenoxy)-N-{2-[rac-(2R,4R)-6-chloro-4-hydroxy-3,4-dihydro-2H-1-benzopyran-2-carbonyl]-2-azaspiro[3.3]heptan-6-yl}acetamide;
2-(4-chloro-3-fluorophenoxy)-N-[2-(6-chloro-4-oxo-3,4-dihydro-2H-1-benzopyran-2-carbonyl)-2-azaspiro[3.3]heptan-6-yl]acetamide;
6-chloro-N-[(3S)-3-hydroxy-4-{[(1s,3R)-3-(trifluoromethoxy)cyclobutane-1-carbonyl]amino}bicyclo[2.2.2]octan-1-yl]-4-oxo-4H-1-benzopyran-2-carboxamide;
(2S,4S)-6-chloro-4-hydroxy-N-[(3S)-3-hydroxy-4-{[(1s,3R)-3-(trifluoromethoxy)cyclobutane-1-carbonyl]amino}bicyclo[2.2.2]octan-1-yl]-3,4-dihydro-2H-1-benzopyran-2-carboxamide and (2R,4R)-6-chloro-4-hydroxy-N-[(3S)-3-hydroxy-4-{[(1s,3R)-3-(trifluoromethoxy)cyclobutane-1-carbonyl]amino}bicyclo[2.2.2]octan-1-yl]-3,4-dihydro-2H-1-benzopyran-2-carboxamide;
6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide;
rac-(2R,4R)-6-chloro-N-{3-[4-(3,4-difluorophenyl)-1H-imidazol-1-yl]bicyclo[1.1.1]pentan-1-yl}-4-hydroxy-3,4-dihydro-2H-1-benzopyran-2-carboxamide;
6-chloro-4-oxo-N-(4-{5-[(1s,3s)-3-(trifluoromethoxy)cyclobutyl]-1,3,4-oxadiazol-2-yl}bicyclo[2.1.1]hexan-1-yl)-3,4-dihydro-2H-1-benzopyran-2-carboxamide;
6-chloro-4-oxo-N-(3-{5-[(1s,3s)-3-(trifluoromethoxy)cyclobutyl]-1,3,4-oxadiazol-2-yl}bicyclo[1.1.1]pentan-1-yl)-3,4-dihydro-2H-1-benzopyran-2-carboxamide;
6-chloro-4-oxo-N-[(3R,6S)-6-{5-[(1s,3R)-3-(trifluoromethoxy)cyclobutyl]-1,3,4-oxadiazol-2-yl}oxan-3-yl]-3,4-dihydro-2H-1-benzopyran-2-carboxamide;
2-(4-chloro-3-fluorophenoxy)-N-[(3R,6S)-6-{5-[(1s,3R)-3-(trifluoromethoxy)cyclobutyl]-1,3,4-oxadiazol-2-yl}oxan-3-yl]acetamide;
(2R,4R)-6-chloro-N-(3-{3-[(4-chloro-3-fluorophenoxy)methyl]-4,5-dihydro-1,2,4-oxadiazol-5-yl}bicyclo[1.1.1]pentan-1-yl)-4-hydroxy-3,4-dihydro-2H-1-benzopyran-2-carboxamide;
(2R)-6-chloro-N-(3-{3-[(4-chloro-3-fluorophenoxy)methyl]-1,2,4-oxadiazol-5-yl}bicyclo[1.1.1]pentan-1-yl)-4-oxo-3,4-dihydro-2H-1-benzopyran-2-carboxamide;
(2R,4R)-6-chloro-N-(3-{3-[(4-chloro-3-fluorophenoxy)methyl]-1,2,4-oxadiazol-5-yl}bicyclo[1.1.1]pentan-1-yl)-4-hydroxy-3,4-dihydro-2H-1-benzopyran-2-carboxamide;
4-(2-{[(1s,3s)-3-(trifluoromethoxy)cyclobutyl]oxy}acetamido)-N-{[5-(trifluoromethyl)pyridin-2-yl]methyl}bicyclo[2.2.2]octane-1-carboxamide;
(1r,4r)-4-(2-{[(1s,3s)-3-(trifluoromethoxy)cyclobutyl]oxy}acetamido)-N-{[5-(trifluoromethyl)pyridin-2-yl]methyl}cyclohexane-1-carboxamide;
rac-(2R,4R)-6-chloro-4-hydroxy-N-(3-{5-[(1s,3S)-3-(trifluoromethoxy)cyclobutyl]-1,3,4-oxadiazol-2-yl}bicyclo[1.1.1]pentan-1-yl)-3,4-dihydro-2H-1-benzopyran-2-carboxamide;
rac-(2R,4R)-6-chloro-4-hydroxy-N-(4-{5-[(1s,3S)-3-(trifluoromethoxy)cyclobutyl]-1,3,4-oxadiazol-2-yl}bicyclo[2.1.1]hexan-1-yl)-3,4-dihydro-2H-1-benzopyran-2-carboxamide;
(2RS,4RS)-6-chloro-4-hydroxy-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide;
(2R)-6-chloro-4-oxo-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide;
6-chloro-4-oxo-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide;
(2R,4R)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide;
6-chloro-4-hydroxy-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide;
2-(4-chloro-3-fluorophenoxy)-N-(3-{5-[rac-(2R,4R)-6-chloro-4-hydroxy-3,4-dihydro-2H-1-benzopyran-2-yl]-1,3,4-oxadiazol-2-yl}bicyclo[1.1.1]pentan-1-yl)acetamide;
6-chloro-4-oxo-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide;
2-(4-chloro-3-fluorophenoxy)-N-[rac-(1R,2S,4R,5S)-5-{5-[cis-3-(trifluoromethoxy)cyclobutyl]-1,3,4-oxadiazol-2-yl}-7-oxabicyclo[2.2.1]heptan-2-yl]acetamide;
(2R,4R)-6-chloro-4-hydroxy-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide;
(2S,4S)-6-chloro-4-hydroxy-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide;
rac-(2R,4R)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide;
(2S,4R)-6-chloro-4-hydroxy-N-[trans-4-({[5-(trifluoromethyl)pyridin-2-yl]methyl}carbamoyl)cyclohexyl]-3,4-dihydro-2H-1-benzopyran-2-carboxamide;
(2R)-6-chloro-N-{trans-4-[3-(4-chlorophenyl)azetidine-1-carbonyl]cyclohexyl}-4-oxo-3,4-dihydro-2H-1-benzopyran-2-carboxamide;
(2R,4R)-6-chloro-N-{trans-4-[3-(4-chlorophenyl)azetidine-1-carbonyl]cyclohexyl}-4-hydroxy-3,4-dihydro-2H-1-benzopyran-2-carboxamide;
(2S)-6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide;
(2R)-6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide;
(2S)-6-chloro-4-oxo-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide;
(2S,4R)-6-chloro-N-{trans-4-[3-(4-chlorophenyl)azetidine-1-carbonyl]cyclohexyl}-4-hydroxy-3,4-dihydro-2H-1-benzopyran-2-carboxamide;
(2R)-6-chloro-N-{3-[3-(4-chlorophenyl)-2-oxoimidazolidin-1-yl]bicyclo[1.1.1]pentan-1-yl}-4-oxo-3,4-dihydro-2H-1-benzopyran-2-carboxamide;
(2S,4S)-6-chloro-N-{3-[4-(3,4-difluorophenyl)-1H-imidazol-1-yl]bicyclo[1.1.1]pentan-1-yl}-4-hydroxy-3,4-dihydro-2H-1-benzopyran-2-carboxamide;
(2R,4R)-6-chloro-N-{3-[4-(3,4-difluorophenyl)-1H-imidazol-1-yl]bicyclo[1.1.1]pentan-1-yl}-4-hydroxy-3,4-dihydro-2H-1-benzopyran-2-carboxamide;
(2R,4R)-6-chloro-4-hydroxy-N-[trans-4-(3-phenylazetidine-1-carbonyl)cyclohexyl]-3,4-dihydro-2H-1-benzopyran-2-carboxamide;
(2R,4R)-6-chloro-N-{3-[3-(4-chlorophenyl)-2-oxoimidazolidin-1-yl]bicyclo[1.1.1]pentan-1-yl}-4-hydroxy-3,4-dihydro-2H-1-benzopyran-2-carboxamide;
(2R)-6-chloro-4-oxo-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide;
(2S,4R)-6-chloro-4-hydroxy-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide;
(2S,4S)-6-chloro-4-hydroxy-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide;
(2R,4R)-6-chloro-4-hydroxy-N-[1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide;
(2R)-6-chloro-4-oxo-N-[(1r,4R)-4-{2-oxo-3-[3-(trifluoromethoxy)cyclobutyl]imidazolidin-1-yl}cyclohexyl]-3,4-dihydro-2H-1-benzopyran-2-carboxamide;
(2R)-6,7-difluoro-4-oxo-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2S,4S)-6-chloro-4-hydroxy-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide;
(2R,4R)-6-chloro-4-hydroxy-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide;
(2R)-6-chloro-4-oxo-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2S,4R)-6-chloro-4-hydroxy-N-[4-({[5-(trifluoromethyl)pyridin-2-yl]methyl}carbamoyl)bicyclo[2.2.2]octan-1-yl]-3,4-dihydro-2H-1-benzopyran-2-carboxamide;
(2R,4R)-6,7-difluoro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R,4R)-6-chloro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R,4R)-6-chloro-4-hydroxy-N-[4-(2-{[cis-3-(trifluoromethoxy)cyclobutyl]oxy}acetamido)bicyclo[2.1.1]hexan-1-yl]-3,4-dihydro-2H-1-benzopyran-2-carboxamide;
(2R,4R)-6-chloro-4-hydroxy-N-[(1r,4R)-4-{2-oxo-3-[3-(trifluoromethoxy)cyclobutyl]imidazolidin-1-yl}cyclohexyl]-3,4-dihydro-2H-1-benzopyran-2-carboxamide;
(2R,4S)-6-chloro-4-hydroxy-N-[trans-4-({[5-(trifluoromethyl)pyridin-2-yl]methyl}carbamoyl)cyclohexyl]-3,4-dihydro-2H-1-benzopyran-2-carboxamide;
(2S,4S)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide;
(2S,4S)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide;
(2R,4R)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide;
(2R,4R)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide;
(2R,4R)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide;
(2S,4S)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide;
(2R,4R)-6-chloro-4-hydroxy-N-(1-{[cis-3-(trifluoromethoxy)cyclobutyl]carbamoyl}-2-oxabicyclo[2.2.2]octan-4-yl)-3,4-dihydro-2H-1-benzopyran-2-carboxamide;
(2S,4S)-6-chloro-4-hydroxy-N-(1-{[cis-3-(trifluoromethoxy)cyclobutyl]carbamoyl}-2-oxabicyclo[2.2.2]octan-4-yl)-3,4-dihydro-2H-1-benzopyran-2-carboxamide;
(2R,4R)-6-chloro-N-{trans-4-[3-(4-chloro-3-fluorophenyl)-2-oxoimidazolidin-1-yl]cyclohexyl}-4-hydroxy-3,4-dihydro-2H-1-benzopyran-2-carboxamide;
(2S,4R)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide;
(2R)-6-chloro-N-{3-[4-(4-chlorophenyl)-1H-pyrazol-1-yl]bicyclo[1.1.1]pentan-1-yl}-4-oxo-3,4-dihydro-2H-1-benzopyran-2-carboxamide;
(2R,4R)-6-chloro-4-hydroxy-N-[(1R*,2S*,4R*,5S*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide;
(2R,4R)-6-chloro-4-hydroxy-N-[(1S*,2R*,4S*,5R*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide;
(2R,4R)-6-chloro-N-{3-[4-(4-chlorophenyl)-1H-pyrazol-1-yl]bicyclo[1.1.1]pentan-1-yl}-4-hydroxy-3,4-dihydro-2H-1-benzopyran-2-carboxamide;
(2R)-6-chloro-4-oxo-N-[trans-4-(2-{[cis-3-(trifluoromethoxy)cyclobutyl]oxy}acetamido)cyclohexyl]-3,4-dihydro-2H-1-benzopyran-2-carboxamide;
(2R,4R)-6-chloro-4-hydroxy-N-[trans-4-(2-{[cis-3-(trifluoromethoxy)cyclobutyl]oxy}acetamido)cyclohexyl]-3,4-dihydro-2H-1-benzopyran-2-carboxamide;
(2R,4R)-6-chloro-4-hydroxy-N-[trans-4-{[cis-3-(trifluoromethoxy)cyclobutyl]carbamoyl}cyclohexyl]-3,4-dihydro-2H-1-benzopyran-2-carboxamide;
(2R)-6-chloro-4-oxo-N-(3-{[cis-3-(trifluoromethoxy)cyclobutyl]carbamoyl}bicyclo[1.1.1]pentan-1-yl)-3,4-dihydro-2H-1-benzopyran-2-carboxamide;
(2S,4R)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide;
(2S,4S)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide;
(2R,4R)-6-chloro-4-hydroxy-N-[(1RS,2SR,4RS,5SR)-5-{[cis-3-(trifluoromethoxy)cyclobutyl]carbamoyl}-7-oxabicyclo[2.2.1]heptan-2-yl]-3,4-dihydro-2H-1-benzopyran-2-carboxamide;
(2R,4R)-6-chloro-4-hydroxy-N-[(2S)-2-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R)-6-chloro-4-oxo-N-(4-{[cis-3-(trifluoromethoxy)cyclobutyl]carbamoyl}bicyclo[2.2.2]octan-1-yl)-3,4-dihydro-2H-1-benzopyran-2-carboxamide;
(2R,4R)-6-chloro-4-hydroxy-N-(4-{[cis-3-(trifluoromethoxy)cyclobutyl]carbamoyl}bicyclo[2.2.2]octan-1-yl)-3,4-dihydro-2H-1-benzopyran-2-carboxamide;
(2R)-6-chloro-N-[(2S)-2-hydroxy-4-(2-{[cis-3-(trifluoromethoxy)cyclobutyl]oxy}acetamido)bicyclo[2.2.2]octan-1-yl]-4-oxo-3,4-dihydro-2H-1-benzopyran-2-carboxamide;
(2R)-6-chloro-N-{3-[3-(4-chlorophenyl)-2-oxopyrrolidin-1-yl]bicyclo[1.1.1]pentan-1-yl}-4-oxo-3,4-dihydro-2H-1-benzopyran-2-carboxamide;
(2R,4R)-6-chloro-N-{3-[(3R*)-3-(4-chlorophenyl)-2-oxopyrrolidin-1-yl]bicyclo[1.1.1]pentan-1-yl}-4-hydroxy-3,4-dihydro-2H-1-benzopyran-2-carboxamide;
(2R,4R)-6-chloro-N-{3-[(3S*)-3-(4-chlorophenyl)-2-oxopyrrolidin-1-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{5-[cis-3-hydroxycyclobutyl]-4,5-dihydro-1,2-oxazol-3-yl}bicyclo[1.1.1]pentan-1-yl)-3,4-dihydro-2H-1-benzopyran-2-carboxamide;
(2S,4R)-6-chloro-4-hydroxy-N-(3-{5-[cis-3-hydroxycyclobutyl]-4,5-dihydro-1,2-oxazol-3-yl}bicyclo[1.1.1]pentan-1-yl)-3,4-dihydro-2H-1-benzopyran-2-carboxamide;
(2R,4R)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide;
(2S,4R)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide;
(2S,4R)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide;
(2R,4R)-6-chloro-4-hydroxy-N-(3-{5-[cis-3-(trifluoromethoxy)cyclobutyl]-4,5-dihydro-1,2-oxazol-3-yl}bicyclo[1.1.1]pentan-1-yl)-3,4-dihydro-2H-1-benzopyran-2-carboxamide;
(2S,4R)-6-chloro-4-hydroxy-N-(3-{5-[cis-3-(trifluoromethoxy)cyclobutyl]-4,5-dihydro-1,2-oxazol-3-yl}bicyclo[1.1.1]pentan-1-yl)-3,4-dihydro-2H-1-benzopyran-2-carboxamide;
(2R,4R)-6-chloro-4-hydroxy-N-(3-{5-[cis-3-(trifluoromethoxy)cyclobutyl]-1,2-oxazol-3-yl}bicyclo[1.1.1]pentan-1-yl)-3,4-dihydro-2H-1-benzopyran-2-carboxamide;
(2R,4R)-6-chloro-N-[3-(5-chloro-1H-indazol-1-yl)bicyclo[1.1.1]pentan-1-yl]-4-hydroxy-3,4-dihydro-2H-1-benzopyran-2-carboxamide;
(2S,4R)-6-chloro-N-{3-[4-(4-chlorophenyl)-1H-pyrazol-1-yl]bicyclo[1.1.1]pentan-1-yl}-4-hydroxy-3,4-dihydro-2H-1-benzopyran-2-carboxamide;
(2R,4R)-6-chloro-N-{3-[1-(4-chloro-3-fluorophenyl)-1H-pyrazol-4-yl]bicyclo[1.1.1]pentan-1-yl}-4-hydroxy-3,4-dihydro-2H-1-benzopyran-2-carboxamide;
(2S,4R)-6-chloro-N-{3-[I-(4-chloro-3-fluorophenyl)-1H-pyrazol-4-yl]bicyclo[1.1.1]pentan-1-yl}-4-hydroxy-3,4-dihydro-2H-1-benzopyran-2-carboxamide;
(2S,4R)-6-chloro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R,4R)-6-chloro-N-{3-[3-(4-chlorophenyl)-1H-pyrrol-1-yl]bicyclo[1.1.1]pentan-1-yl}-4-hydroxy-3,4-dihydro-2H-1-benzopyran-2-carboxamide;
(2S,4R)-6-chloro-N-{3-[3-(4-chlorophenyl)-1H-pyrrol-1-yl]bicyclo[1.1.1]pentan-1-yl}-4-hydroxy-3,4-dihydro-2H-1-benzopyran-2-carboxamide;
(2R,4R)-6-chloro-N-{3-[3-(4-chloro-3-fluorophenyl)-1H-pyrrol-1-yl]bicyclo[1.1.1]pentan-1-yl}-4-hydroxy-3,4-dihydro-2H-1-benzopyran-2-carboxamide;
(2S,4R)-6-chloro-N-{3-[3-(4-chloro-3-fluorophenyl)-1H-pyrrol-1-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{3-[6-(trifluoromethyl)pyridin-3-yl]-1H-pyrrol-1-yl}bicyclo[1.1.1]pentan-1-yl)-3,4-dihydro-2H-1-benzopyran-2-carboxamide;
(2S,4R)-6-chloro-4-hydroxy-N-(3-{3-[6-(trifluoromethyl)pyridin-3-yl]-1H-pyrrol-1-yl}bicyclo[1.1.1]pentan-1-yl)-3,4-dihydro-2H-1-benzopyran-2-carboxamide;
(2R,4R)-6-chloro-N-{3-[3-(4-chlorophenyl)-1,2-oxazol-5-yl]bicyclo[1.1.1]pentan-1-yl}-4-hydroxy-3,4-dihydro-2H-1-benzopyran-2-carboxamide;
(2S,4R)-6-chloro-N-{3-[3-(4-chlorophenyl)-1,2-oxazol-5-yl]bicyclo[1.1.1]pentan-1-yl}-4-hydroxy-3,4-dihydro-2H-1-benzopyran-2-carboxamide;
(2R,4R)-6-chloro-N-{3-[3-(4-chloro-3-fluorophenyl)-1,2-oxazol-5-yl]bicyclo[1.1.1]pentan-1-yl}-4-hydroxy-3,4-dihydro-2H-1-benzopyran-2-carboxamide;
(2S,4R)-6-chloro-N-{3-[3-(4-chloro-3-fluorophenyl)-1,2-oxazol-5-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{3-[6-(trifluoromethyl)pyridin-3-yl]-1,2-oxazol-5-yl}bicyclo[1.1.1]pentan-1-yl)-3,4-dihydro-2H-1-benzopyran-2-carboxamide;
(2S,4R)-6-chloro-4-hydroxy-N-(3-{3-[6-(trifluoromethyl)pyridin-3-yl]-1,2-oxazol-5-yl}bicyclo[1.1.1]pentan-1-yl)-3,4-dihydro-2H-1-benzopyran-2-carboxamide;
2-(4-chloro-3-fluorophenoxy)-N-(3-{5-[(2R*,4R*)-6-chloro-4-hydroxy-3,4-dihydro-2H-1-benzopyran-2-yl]-1,3,4-oxadiazol-2-yl}bicyclo[1.1.1]pentan-1-yl)acetamide;
2-(4-chloro-3-fluorophenoxy)-N-{3-(5-[(2S*,4S*)-6-chloro-4-hydroxy-3,4-dihydro-2H-1-benzopyran-2-yl]-1,3,4-oxadiazol-2-yl}bicyclo[1.1.1]pentan-1-yl)acetamide;
(2R,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide;
(2S,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide;
(2R,4R)-6-chloro-N-{3-[5-(4-chlorophenyl)-1,2-oxazol-3-yl]bicyclo[1.1.1]pentan-1-yl}-4-hydroxy-3,4-dihydro-2H-1-benzopyran-2-carboxamide;
(2S,4R)-6-chloro-N-{3-[5-(4-chlorophenyl)-1,2-oxazol-3-yl]bicyclo[1.1.1]pentan-1-yl}-4-hydroxy-3,4-dihydro-2H-1-benzopyran-2-carboxamide;
(2R,4R)-6-chloro-N-{3-[5-(4-chloro-3-fluorophenyl)-1,2-oxazol-3-yl]bicyclo[1.1.1]pentan-1-yl}-4-hydroxy-3,4-dihydro-2H-1-benzopyran-2-carboxamide;
(2S,4R)-6-chloro-N-{3-[5-(4-chloro-3-fluorophenyl)-1,2-oxazol-3-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{5-[6-(trifluoromethyl)pyridin-3-yl]-1,2-oxazol-3-yl}bicyclo[1.1.1]pentan-1-yl)-3,4-dihydro-2H-1-benzopyran-2-carboxamide;
(2S,4R)-6-chloro-4-hydroxy-N-(3-{5-[6-(trifluoromethyl)pyridin-3-yl]-1,2-oxazol-3-yl}bicyclo[1.1.1]pentan-1-yl)-3,4-dihydro-2H-1-benzopyran-2-carboxamide;
(2R,4R)-6-chloro-4-hydroxy-N-(3-{1-[6-(trifluoromethyl)pyridin-3-yl]-1H-pyrazol-4-yl}bicyclo[1.1.1]pentan-1-yl)-3,4-dihydro-2H-1-benzopyran-2-carboxamide;
(2S,4R)-6-chloro-4-hydroxy-N-(3-{1-[6-(trifluoromethyl)pyridin-3-yl]-1H-pyrazol-4-yl}bicyclo[1.1.1]pentan-1-yl)-3,4-dihydro-2H-1-benzopyran-2-carboxamide;
(2R,4R)-6-chloro-N-{3-[1-(4-chlorophenyl)-1H-pyrazol-4-yl]bicyclo[1.1.1]pentan-1-yl}-4-hydroxy-3,4-dihydro-2H-1-benzopyran-2-carboxamide;
(2S,4R)-6-chloro-N-{3-[1-(4-chlorophenyl)-1H-pyrazol-4-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{3-[cis-3-(trifluoromethoxy)cyclobutyl]-1,2-oxazol-5-yl}bicyclo[1.1.1]pentan-1-yl)-3,4-dihydro-2H-1-benzopyran-2-carboxamide;
(2R,4R)-6-chloro-4-hydroxy-N-(3-{2-oxo-5-[cis-3-(trifluoromethoxy)cyclobutyl]-1,3-oxazolidin-3-yl}bicyclo[1.1.1]pentan-1-yl)-3,4-dihydro-2H-1-benzopyran-2-carboxamide;
(2S,4R)-6-chloro-4-hydroxy-N-(3-{3-[cis-3-(trifluoromethoxy)cyclobutyl]-1,2-oxazol-5-yl}bicyclo[1.1.1]pentan-1-yl)-3,4-dihydro-2H-1-benzopyran-2-carboxamide;
(2S,4R)-6-chloro-4-hydroxy-N-(3-{2-oxo-5-[cis-3-(trifluoromethoxy)cyclobutyl]-1,3-oxazolidin-3-yl}bicyclo[1.1.1]pentan-1-yl)-3,4-dihydro-2H-1-benzopyran-2-carboxamide;
(2R,4R)-6-chloro-N-{3-[5-(4-chloro-3-fluorophenyl)-1,3-oxazol-2-yl]bicyclo[1.1.1]pentan-1-yl}-4-hydroxy-3,4-dihydro-2H-1-benzopyran-2-carboxamide;
(2S,4R)-6-chloro-N-{3-[5-(4-chloro-3-fluorophenyl)-1,3-oxazol-2-yl]bicyclo[1.1.1]pentan-1-yl}-4-hydroxy-3,4-dihydro-2H-1-benzopyran-2-carboxamide;
(2R,4R)-6-chloro-N-{3-[2-(4-chlorophenyl)-1,3-thiazol-4-yl]bicyclo[1.1.1]pentan-1-yl}-4-hydroxy-3,4-dihydro-2H-1-benzopyran-2-carboxamide;
(2S,4R)-6-chloro-N-{3-[2-(4-chlorophenyl)-1,3-thiazol-4-yl]bicyclo[1.1.1]pentan-1-yl}-4-hydroxy-3,4-dihydro-2H-1-benzopyran-2-carboxamide;
(2R,4R)-6-chloro-N-{3-[5-(4-chlorophenyl)-4-methyl-1,3-oxazol-2-yl]bicyclo[1.1.1]pentan-1-yl}-4-hydroxy-3,4-dihydro-2H-1-benzopyran-2-carboxamide;
(2S,4R)-6-chloro-N-{3-[5-(4-chlorophenyl)-4-methyl-1,3-oxazol-2-yl]bicyclo[1.1.1]pentan-1-yl}-4-hydroxy-3,4-dihydro-2H-1-benzopyran-2-carboxamide;
(2S,4S)-6-chloro-4-hydroxy-N-[(3S)-3-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R,4R)-6-chloro-N-{3-[5-(4-chlorophenyl)-2-oxo-1,3-oxazolidin-3-yl]bicyclo[1.1.1]pentan-1-yl}-4-hydroxy-3,4-dihydro-2H-1-benzopyran-2-carboxamide;
(2S,4S)-6-chloro-N-{3-[5-(4-chlorophenyl)-2-oxo-1,3-oxazolidin-3-yl]bicyclo[1.1.1]pentan-1-yl}-4-hydroxy-3,4-dihydro-2H-1-benzopyran-2-carboxamide;
(2S,4R)-6-chloro-4-hydroxy-N-[(3S)-3-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide;
(2R,4R)-6-chloro-4-hydroxy-N-(3-{2-[cis-3-(trifluoromethoxy)cyclobutyl]-1,3-thiazol-4-yl}bicyclo[1.1.1]pentan-1-yl)-3,4-dihydro-2H-1-benzopyran-2-carboxamide;
(2R,4R)-6-chloro-N-{3-[4-(4-chlorophenyl)-1H-imidazol-1-yl]bicyclo[1.1.1]pentan-1-yl}-4-hydroxy-3,4-dihydro-2H-1-benzopyran-2-carboxamide;
(2R,4R)-6-chloro-N-{3-[4-(4-chloro-3-fluorophenyl)-1H-imidazol-1-yl]bicyclo[1.1.1]pentan-1-yl}-4-hydroxy-3,4-dihydro-2H-1-benzopyran-2-carboxamide;
(2S,4R)-6-chloro-4-hydroxy-N-{3-(5-[cis-3-(trifluoromethoxy)cyclobutyl]-1,2-oxazol-3-yl}bicyclo[1.1.1]pentan-1-yl)-3,4-dihydro-2H-1-benzopyran-2-carboxamide;
(2R,4R)-6-chloro-4-hydroxy-N-(3-{3-[trans-3-(trifluoromethoxy)cyclobutyl]-1,2-oxazol-5-yl}bicyclo[1.1.1]pentan-1-yl)-3,4-dihydro-2H-1-benzopyran-2-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-phenyl-1,3-oxazole-5-carboxamide;
(2R,4R)-6-chloro-N-[3-(2-{[cis-3-cyanocyclobutyl]oxy}-1,3-thiazol-4-yl)bicyclo[1.1.1]pentan-1-yl]-4-hydroxy-3,4-dihydro-2H-1-benzopyran-2-carboxamide;
(2R,4R)-6-chloro-4-hydroxy-N-(3-{4-[cis-3-(trifluoromethoxy)cyclobutyl]-1H-imidazol-1-yl}bicyclo[1.1.1]pentan-1-yl)-3,4-dihydro-2H-1-benzopyran-2-carboxamide;
(2R,4R)-6-chloro-4-hydroxy-N-(3-{5-[cis-3-(trifluoromethoxy)cyclobutyl]-1,3-oxazol-2-yl}bicyclo[1.1.1]pentan-1-yl)-3,4-dihydro-2H-1-benzopyran-2-carboxamide;
(2R,4R)-6-chloro-4-hydroxy-N-(3-{5-[cis-3-(trifluoromethoxy)cyclobutyl]-1H-imidazol-2-yl}bicyclo[1.1.1]pentan-1-yl)-3,4-dihydro-2H-1-benzopyran-2-carboxamide;
(2R,4R)-6-chloro-N-[3-(4-cyclobutyl-1H-pyrazol-1-yl)bicyclo[1.1.1]pentan-1-yl]-4-hydroxy-3,4-dihydro-2H-1-benzopyran-2-carboxamide;
(2S,4R)-6-chloro-N-[3-(4-cyclobutyl-1H-pyrazol-1-yl)bicyclo[1.1.1]pentan-1-yl]-4-hydroxy-3,4-dihydro-2H-1-benzopyran-2-carboxamide;
(2R,4R)-6-chloro-4-hydroxy-N-[(3R,6S)-6-{5-[3-(trifluoromethoxy)cyclobutyl]-1,3-oxazol-2-yl}oxan-3-yl]-3,4-dihydro-2H-1-benzopyran-2-carboxamide;
(2R,4S)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide;
(2R,4R)-6-chloro-4-hydroxy-N-(3-{1-[cis-3-(trifluoromethoxy)cyclobutyl]-1H-imidazol-4-yl}bicyclo[1.1.1]pentan-1-yl)-3,4-dihydro-2H-1-benzopyran-2-carboxamide;
(2S,4S)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide;
(2R)-6-chloro-4-oxo-N-[3-({(1RS,2SR)-2-[(trifluoromethoxy)methyl]cyclopropane-1-carbonyl}amino)bicyclo[1.1.1]pentan-1-yl]-3,4-dihydro-2H-1-benzopyran-2-carboxamide;
(2R,4R)-6-chloro-4-hydroxy-N-(4-{5-[cis-3-(trifluoromethoxy)cyclobutyl]-1,3-oxazol-2-yl}bicyclo[2.2.2]octan-1-yl)-3,4-dihydro-2H-1-benzopyran-2-carboxamide;
(2R,4R)-6-chloro-4-hydroxy-N-(3-{1-[cis-3-(trifluoromethoxy)cyclobutyl]-1H-pyrazol-3-yl}bicyclo[1.1.1]pentan-1-yl)-3,4-dihydro-2H-1-benzopyran-2-carboxamide;
(2R,4R)-6-chloro-4-hydroxy-N-[3-({(1RS,2SR)-2-[(trifluoromethoxy)methyl]cyclopropane-1-carbonyl}amino)bicyclo[1.1.1]pentan-1-yl]-3,4-dihydro-2H-1-benzopyran-2-carboxamide;
(2R,4R)-6-chloro-4-hydroxy-N-[3-(2-{[cis-3-(trifluoromethoxy)cyclobutyl]oxy}-1,3-thiazol-4-yl)bicyclo[1.1.1]pentan-1-yl]-3,4-dihydro-2H-1-benzopyran-2-carboxamide;
(2R,4R)-6-chloro-4-hydroxy-N-[3-({4-[cis-3-(trifluoromethoxy)cyclobutyl]-1,3-thiazol-2-yl}oxy)bicyclo[1.1.1]pentan-1-yl]-3,4-dihydro-2H-1-benzopyran-2-carboxamide;
(2R,4R)-6-chloro-4-hydroxy-N-(3-{4-[4-(trifluoromethoxy)phenyl]-1H-pyrazol-1-yl}bicyclo[1.1.1]pentan-1-yl)-3,4-dihydro-2H-1-benzopyran-2-carboxamide;
(2R,4R)-6-chloro-N-[trans-4-{3-[5-(difluoromethyl)pyrazin-2-yl]-2-oxoimidazolidin-1-yl}cyclohexyl]-4-hydroxy-3,4-dihydro-2H-1-benzopyran-2-carboxamide;
(2R,4R)-6-chloro-N-{(1R,2S,4R,5S)-5-[4-(3,4-difluorophenyl)-1H-imidazol-1-yl]bicyclo[2.2.1]heptan-2-yl}-4-hydroxy-3,4-dihydro-2H-1-benzopyran-2-carboxamide;
(2R,4R)-6-chloro-N-{3-[2-(4-chloro-3-fluorophenyl)-1,3-oxazol-5-yl]bicyclo[1.1.1]pentan-1-yl}-4-hydroxy-3,4-dihydro-2H-1-benzopyran-2-carboxamide;
(2S,4R)-6-chloro-N-(3-{4-[3-fluoro-4-(trifluoromethoxy)phenyl]-1H-pyrazol-1-yl}bicyclo[1.1.1]pentan-1-yl)-4-hydroxy-3,4-dihydro-2H-1-benzopyran-2-carboxamide;
(2R,4R)-6-chloro-N-{3-[4-(4-chlorophenyl)-2-oxopyrrolidin-1-yl]bicyclo[1.1.1]pentan-1-yl}-4-hydroxy-3,4-dihydro-2H-1-benzopyran-2-carboxamide;
(2R,4R)-6-chloro-4-hydroxy-N-(3-{4-[5-(trifluoromethoxy)pyridin-2-yl]-1H-pyrazol-1-yl}bicyclo[1.1.1]pentan-1-yl)-3,4-dihydro-2H-1-benzopyran-2-carboxamide;
(2S,4R)-6-chloro-4-hydroxy-N-[trans-4-{2-oxo-3-[6-(trifluoromethyl)pyridin-3-yl]imidazolidin-1-yl}cyclohexyl]-3,4-dihydro-2H-1-benzopyran-2-carboxamide;
(2R,4R)-6-chloro-4-hydroxy-N-[trans-4-{2-oxo-3-[6-(trifluoromethyl)pyridin-3-yl]imidazolidin-1-yl}cyclohexyl]-3,4-dihydro-2H-1-benzopyran-2-carboxamide;
(2R,4R)-6-chloro-N-{3-[1-(4-chloro-3-fluorophenyl)-1H-1,2,3-triazol-4-yl]bicyclo[1.1.1]pentan-1-yl}-4-hydroxy-3,4-dihydro-2H-1-benzopyran-2-carboxamide;
(2R,4R)-6-chloro-N-(3-{4-[3-fluoro-4-(trifluoromethoxy)phenyl]-1H-pyrazol-1-yl}bicyclo[1.1.1]pentan-1-yl)-4-hydroxy-3,4-dihydro-2H-1-benzopyran-2-carboxamide;
(2R,4R)-6-chloro-4-hydroxy-N-[(1RS,2SR,4RS,5SR)-5-({[5-(trifluoromethyl)pyridin-2-yl]methyl}carbamoyl)-7-oxabicyclo[2.2.1]heptan-2-yl]-3,4-dihydro-2H-1-benzopyran-2-carboxamide;
(2R,4R)-6-chloro-4-hydroxy-N-(3-{3-[cis-3-(trifluoromethoxy)cyclobutyl]-1H-pyrazol-1-yl}bicyclo[1.1.1]pentan-1-yl)-3,4-dihydro-2H-1-benzopyran-2-carboxamide;
(2R,4R)-6-chloro-4-hydroxy-N-(3-{1-[cis-3-(trifluoromethoxy)cyclobutyl]-1H-1,2,3-triazol-4-yl}bicyclo[1.1.1]pentan-1-yl)-3,4-dihydro-2H-1-benzopyran-2-carboxamide;
(2R,4R)-6-chloro-4-hydroxy-N-(3-{1-[cis-3-(trifluoromethoxy)cyclobutyl]-1H-pyrazol-4-yl}bicyclo[1.1.1]pentan-1-yl)-3,4-dihydro-2H-1-benzopyran-2-carboxamide;
   and a pharmaceutically acceptable salt, solvate, hydrate, tautomer, N-oxide, or stereoisomer thereof.

In some embodiments, a compound disclosed herein, or a pharmaceutically acceptable salt thereof is formulated as a pharmaceutically acceptable composition comprising a disclosed compound and a pharmaceutically acceptable carrier.

In some embodiments, a disclosed compound is selected from a compound set forth in Table 1 or a pharmaceutically acceptable salt, solvate, hydrate, tautomer, N-oxide or stereoisomer thereof.

TABLE 1
Exemplary compounds of the invention

| Compound | |
|---|---|
| Number | Structure |
| 100 | 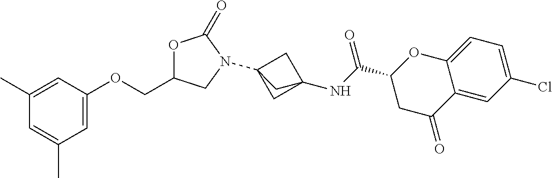 |
| 101 | 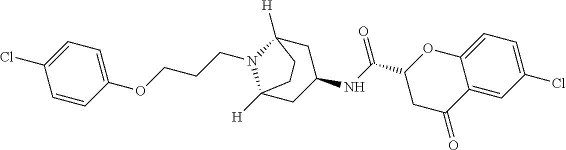 |
| 102 | 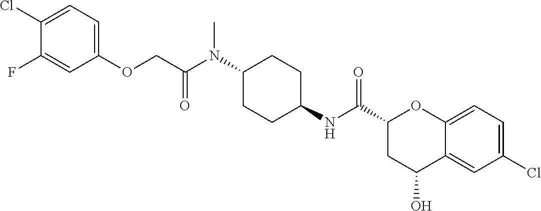 [SMILES: CN(C(=O)COc1ccc(Cl)c(F)c1)[C@H]1CC[C@H](NC(=O)[C@H]2C[C@@H](O)c3cc(Cl)ccc3O2)CC1] |
| 103 | 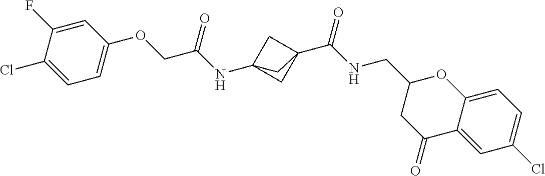 [SMILES: O=C(COc1ccc(Cl)c(F)c1)NC12CC(C(=O)NCC3CC(=O)c4cc(Cl)ccc4O3)(C1)C2] |
| 104 | 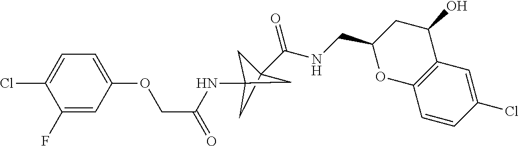 [SMILES: O=C(COc1ccc(Cl)c(F)c1)NC12CC(C(=O)NC[C@H]3C[C@@H](O)c4cc(Cl)ccc4O3)(C1)C2] |
| 105 | 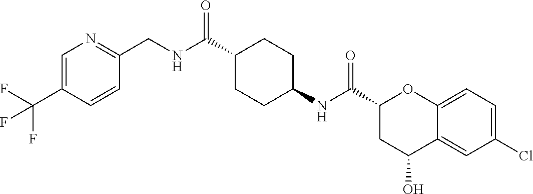 [SMILES: O=C(N[C@H]1CC[C@H](C(=O)NCc2ccc(C(F)(F)F)cn2)CC1)[C@H]1C[C@@H](O)c2cc(Cl)ccc2O1] |
| 106 | 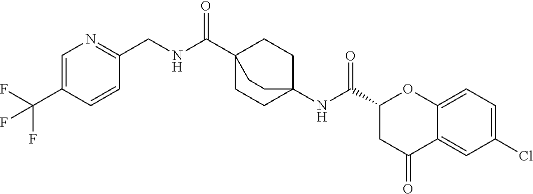 [SMILES: O=C1C[C@H](C(=O)NC23CCC(C(=O)NCc4ccc(C(F)(F)F)cn4)(CC2)CC3)Oc2ccc(Cl)cc21] |
| 107 | 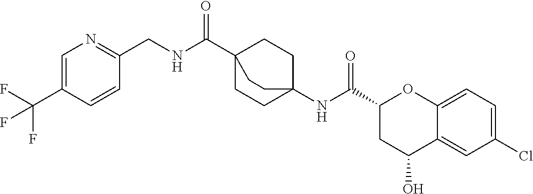 [SMILES: O=C(NC12CCC(C(=O)NCc3ccc(C(F)(F)F)cn3)(CC1)CC2)[C@H]1C[C@@H](O)c2cc(Cl)ccc2O1] |
| 108 | 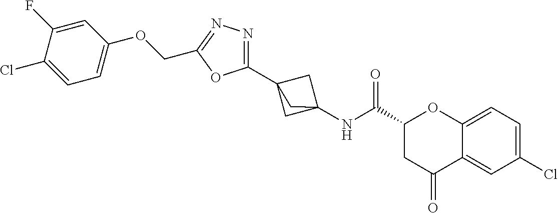 [SMILES: O=C1C[C@H](C(=O)NC23CC(c4nnc(COc5ccc(Cl)c(F)c5)o4)(C2)C3)Oc2ccc(Cl)cc21] |
| 109 | 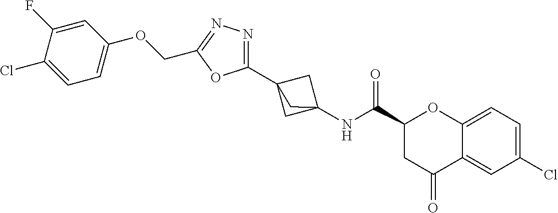 [SMILES: O=C1C[C@@H](C(=O)NC23CC(c4nnc(COc5ccc(Cl)c(F)c5)o4)(C2)C3)Oc2ccc(Cl)cc21] |
| 110 | 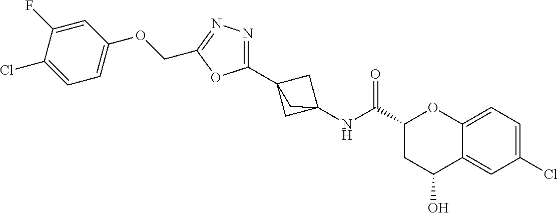 [SMILES: O=C(NC12CC(c3nnc(COc4ccc(Cl)c(F)c4)o3)(C1)C2)[C@H]1C[C@@H](O)c2cc(Cl)ccc2O1] |
| 111 | 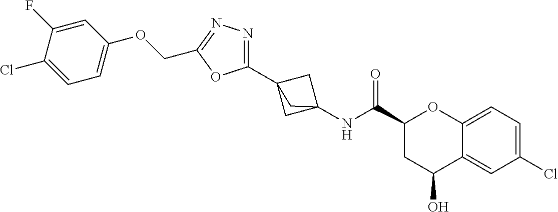 [SMILES: O=C(NC12CC(c3nnc(COc4ccc(Cl)c(F)c4)o3)(C1)C2)[C@@H]1C[C@H](O)c2cc(Cl)ccc2O1] |
| 112 | 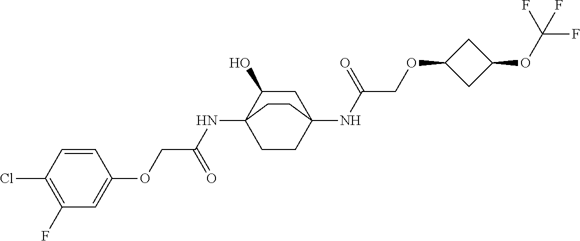 [SMILES: O=C(CO[C@H]1C[C@@H](OC(F)(F)F)C1)NC12CCC(NC(=O)COc3ccc(Cl)c(F)c3)(CC1)[C@@H](O)C2] |
| 113 | 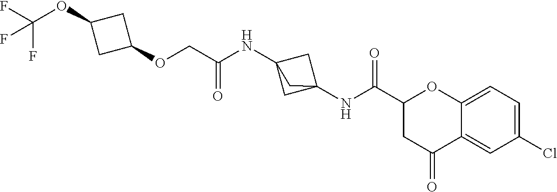 [SMILES: O=C(CO[C@H]1C[C@@H](OC(F)(F)F)C1)NC12CC(NC(=O)C3CC(=O)c4cc(Cl)ccc4O3)(C1)C2] |
| 114 | 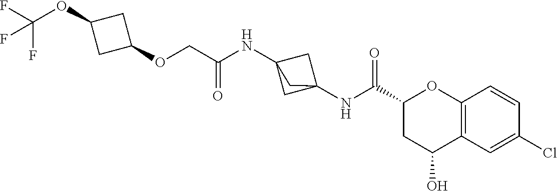 [SMILES: O=C(CO[C@H]1C[C@@H](OC(F)(F)F)C1)NC12CC(NC(=O)[C@H]3C[C@@H](O)c4cc(Cl)ccc4O3)(C1)C2] |
| 115 | 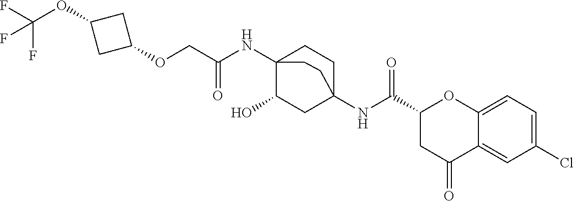 [SMILES: O=C(CO[C@H]1C[C@@H](OC(F)(F)F)C1)NC12CCC(NC(=O)[C@H]3CC(=O)c4cc(Cl)ccc4O3)(CC1)C[C@@H]2O] |
| 116 | 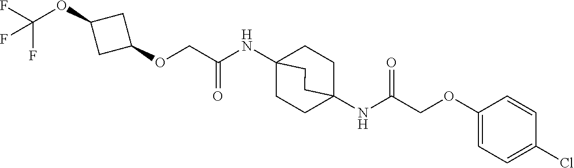 [SMILES: O=C(COc1ccc(Cl)cc1)NC12CCC(NC(=O)CO[C@H]3C[C@@H](OC(F)(F)F)C3)(CC1)CC2] |
| 117 | 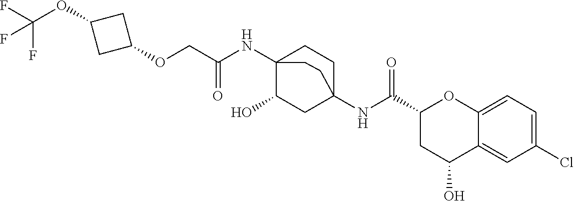 [SMILES: O=C(CO[C@H]1C[C@@H](OC(F)(F)F)C1)NC12CCC(NC(=O)[C@H]3C[C@@H](O)c4cc(Cl)ccc4O3)(CC1)C[C@@H]2O] |
| 118 | 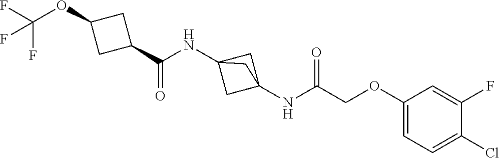 [SMILES: O=C(COc1ccc(Cl)c(F)c1)NC12CC(NC(=O)[C@H]3C[C@@H](OC(F)(F)F)C3)(C1)C2] |
| 119 | 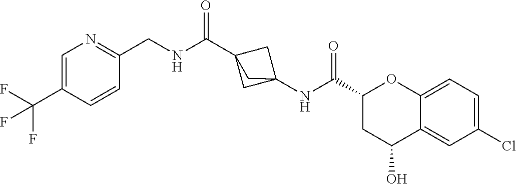 [SMILES: O=C(NC12CC(C(=O)NCc3ccc(C(F)(F)F)cn3)(C1)C2)[C@H]1C[C@@H](O)c2cc(Cl)ccc2O1] |
| 120 | 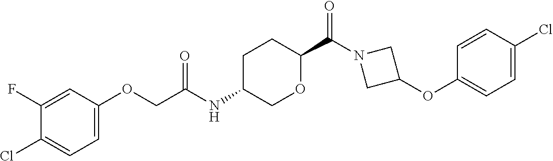 [SMILES: O=C(COc1ccc(Cl)c(F)c1)N[C@@H]1CC[C@@H](C(=O)N2CC(Oc3ccc(Cl)cc3)C2)OC1] |
| 121 | 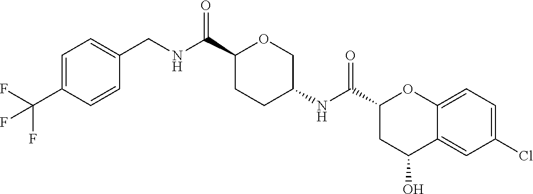 [SMILES: O=C(NCc1ccc(C(F)(F)F)cc1)[C@@H]1CC[C@@H](NC(=O)[C@H]2C[C@@H](O)c3cc(Cl)ccc3O2)CO1] |
| | 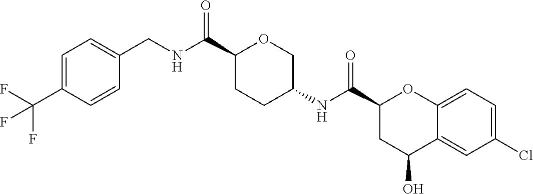 [SMILES: O=C(NCc1ccc(C(F)(F)F)cc1)[C@@H]1CC[C@@H](NC(=O)[C@@H]2C[C@H](O)c3cc(Cl)ccc3O2)CO1] |
| 122 | 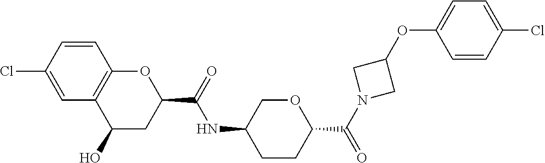 [SMILES: O=C(N[C@@H]1CC[C@@H](C(=O)N2CC(Oc3ccc(Cl)cc3)C2)OC1)[C@H]1C[C@@H](O)c2cc(Cl)ccc2O1] |
| | 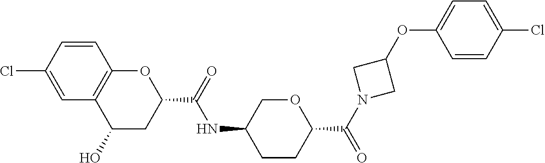 [SMILES: O=C(N[C@@H]1CC[C@@H](C(=O)N2CC(Oc3ccc(Cl)cc3)C2)OC1)[C@@H]1C[C@H](O)c2cc(Cl)ccc2O1] |
| 123 | 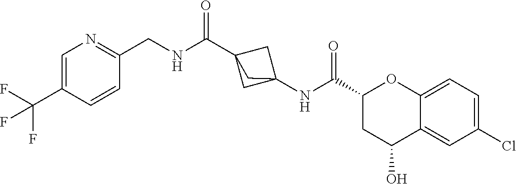 [SMILES: O=C(NC12CC(C(=O)NCc3ccc(C(F)(F)F)cn3)(C1)C2)[C@H]1C[C@@H](O)c2cc(Cl)ccc2O1] |
| 124 | 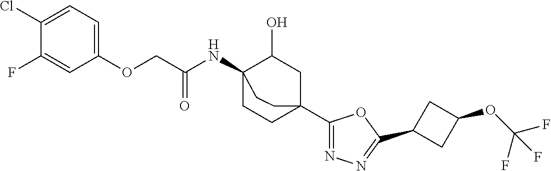 |
| 125 | 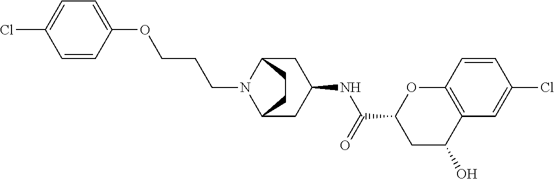 [SMILES: O=C(N[C@H]1C[C@H]2CC[C@@H](C1)N2CCCOc1ccc(Cl)cc1)[C@H]1C[C@@H](O)c2cc(Cl)ccc2O1] |
| 126 | 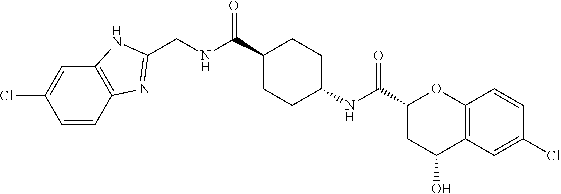 [SMILES: O=C(N[C@H]1CC[C@H](C(=O)NCc2nc3ccc(Cl)cc3[nH]2)CC1)[C@H]1C[C@@H](O)c2cc(Cl)ccc2O1] |
| 127 | 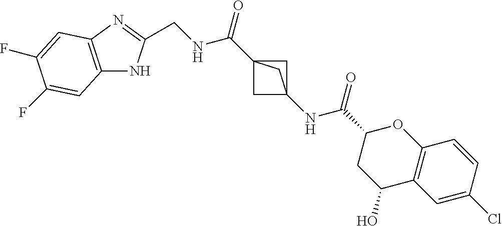 [SMILES: O=C(NC12CC(C(=O)NCc3nc4cc(F)c(F)cc4[nH]3)(C1)C2)[C@H]1C[C@@H](O)c2cc(Cl)ccc2O1] |
| 128 | 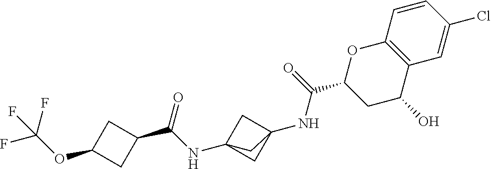 [SMILES: O=C(NC12CC(NC(=O)[C@H]3C[C@@H](OC(F)(F)F)C3)(C1)C2)[C@H]1C[C@@H](O)c2cc(Cl)ccc2O1] |
| 129 | 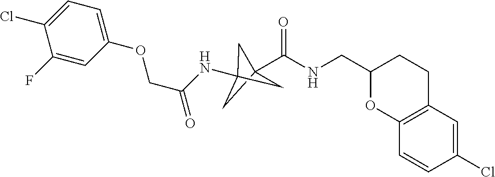 [SMILES: O=C(COc1ccc(Cl)c(F)c1)NC12CC(C(=O)NCC3CCc4cc(Cl)ccc4O3)(C1)C2] |
| 130 | 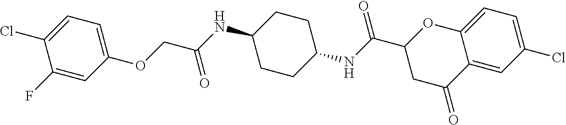 [SMILES: O=C(COc1ccc(Cl)c(F)c1)N[C@H]1CC[C@H](NC(=O)C2CC(=O)c3cc(Cl)ccc3O2)CC1] |
| 131 | 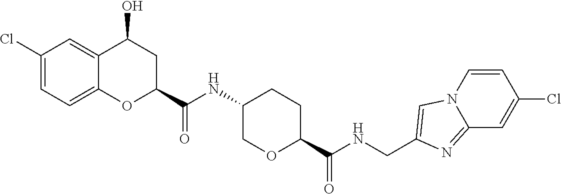 [SMILES: O=C(NCc1cn2ccc(Cl)cc2n1)[C@@H]1CC[C@@H](NC(=O)[C@@H]2C[C@H](O)c3cc(Cl)ccc3O2)CO1] |
| | 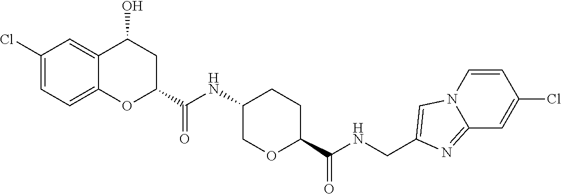 [SMILES: O=C(NCc1cn2ccc(Cl)cc2n1)[C@@H]1CC[C@@H](NC(=O)[C@H]2C[C@@H](O)c3cc(Cl)ccc3O2)CO1] |
| 132 | 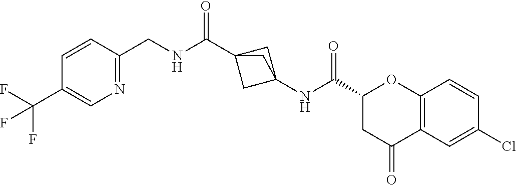 [SMILES: O=C1C[C@H](C(=O)NC23CC(C(=O)NCc4ccc(C(F)(F)F)cn4)(C2)C3)Oc2ccc(Cl)cc21] |
| 133 | 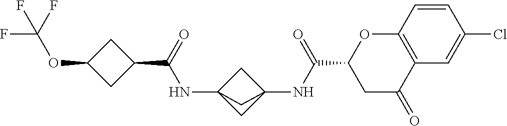 [SMILES: O=C1C[C@H](C(=O)NC23CC(NC(=O)[C@H]4C[C@@H](OC(F)(F)F)C4)(C2)C3)Oc2ccc(Cl)cc21] |
| 134 | 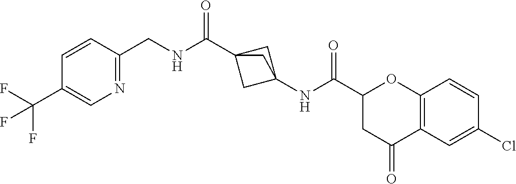 [SMILES: O=C1CC(C(=O)NC23CC(C(=O)NCc4ccc(C(F)(F)F)cn4)(C2)C3)Oc2ccc(Cl)cc21] |
| 135 | 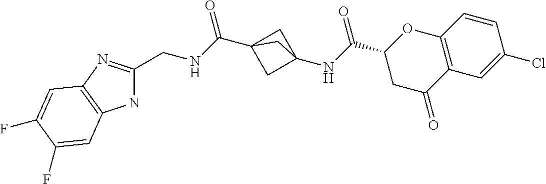 [SMILES: O=C1C[C@H](C(=O)NC23CC(C(=O)NCc4nc5cc(F)c(F)cc5[nH]4)(C2)C3)Oc2ccc(Cl)cc21] |
| 136 | 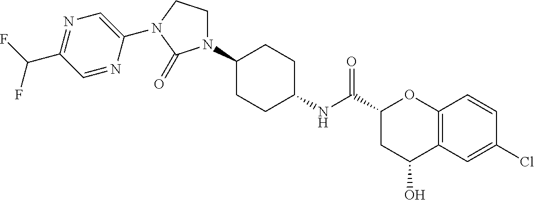 [SMILES: O=C(N[C@H]1CC[C@H](N2CCN(c3cnc(C(F)F)cn3)C2=O)CC1)[C@H]1C[C@@H](O)c2cc(Cl)ccc2O1] |
| 137 | 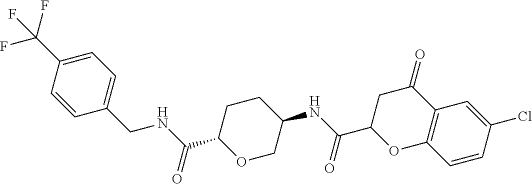 [SMILES: O=C1CC(C(=O)N[C@@H]2CC[C@@H](C(=O)NCc3ccc(C(F)(F)F)cc3)OC2)Oc2ccc(Cl)cc21] |
| 138 | 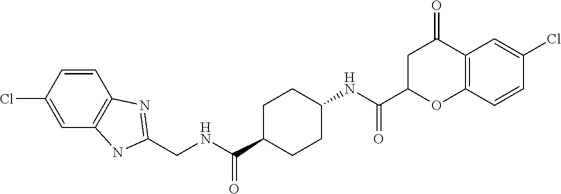 [SMILES: O=C1CC(C(=O)N[C@H]2CC[C@H](C(=O)NCc3nc4ccc(Cl)cc4[nH]3)CC2)Oc2ccc(Cl)cc21] |
| 139 | 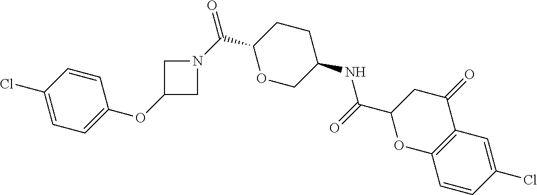 [SMILES: O=C1CC(C(=O)N[C@@H]2CC[C@@H](C(=O)N3CC(Oc4ccc(Cl)cc4)C3)OC2)Oc2ccc(Cl)cc21] |
| 140 | 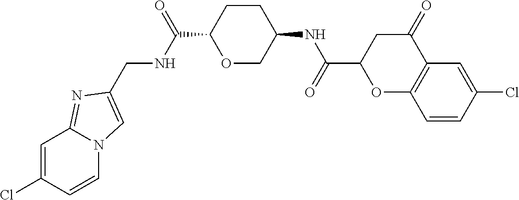 [SMILES: O=C1CC(C(=O)N[C@@H]2CC[C@@H](C(=O)NCc3cn4ccc(Cl)cc4n3)OC2)Oc2ccc(Cl)cc21] |
| 141 | 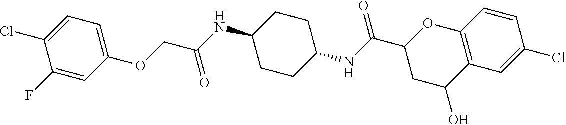 [SMILES: O=C(COc1ccc(Cl)c(F)c1)N[C@H]1CC[C@H](NC(=O)C2CC(O)c3cc(Cl)ccc3O2)CC1] |
| 142 | 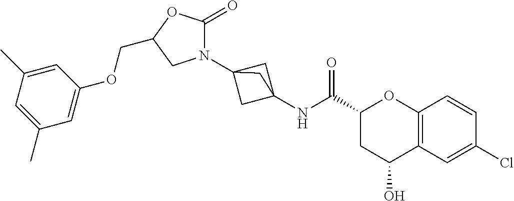 [SMILES: Cc1cc(C)cc(OCC2CN(C34CC(NC(=O)[C@H]5C[C@@H](O)c6cc(Cl)ccc6O5)(C3)C4)C(=O)O2)c1] |
| 143 | 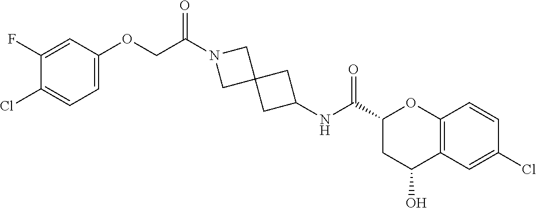 [SMILES: O=C(NC1CC2(C1)CN(C(=O)COc1ccc(Cl)c(F)c1)C2)[C@H]1C[C@@H](O)c2cc(Cl)ccc2O1] |
| 144 | 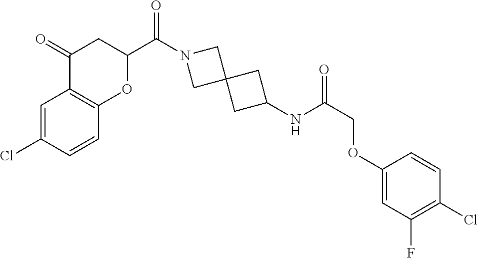 [SMILES: O=C(COc1ccc(Cl)c(F)c1)NC1CC2(C1)CN(C(=O)C1CC(=O)c3cc(Cl)ccc3O1)C2] |
| 145 | 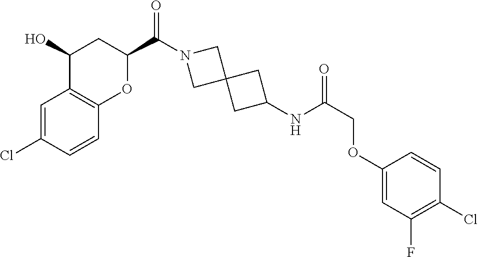 [SMILES: O=C(COc1ccc(Cl)c(F)c1)NC1CC2(C1)CN(C(=O)[C@@H]1C[C@H](O)c3cc(Cl)ccc3O1)C2] |
| 146 | 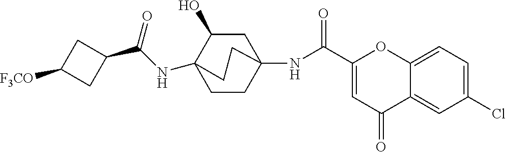 [SMILES: O=C(NC12CCC(NC(=O)[C@H]3C[C@@H](OC(F)(F)F)C3)(CC1)[C@@H](O)C2)c1cc(=O)c2cc(Cl)ccc2o1] |
| 147 | 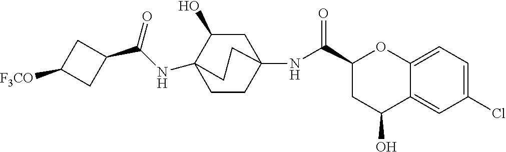 [SMILES: O=C(NC12CCC(NC(=O)[C@H]3C[C@@H](OC(F)(F)F)C3)(CC1)[C@@H](O)C2)[C@@H]1C[C@H](O)c2cc(Cl)ccc2O1] |
| | 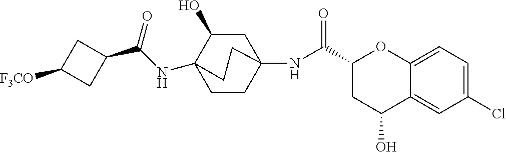 [SMILES: O=C(NC12CCC(NC(=O)[C@H]3C[C@@H](OC(F)(F)F)C3)(CC1)[C@@H](O)C2)[C@H]1C[C@@H](O)c2cc(Cl)ccc2O1] |
| 148 | 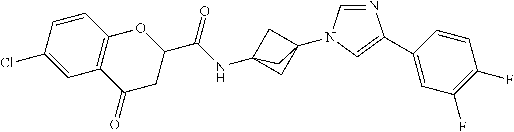 [SMILES: O=C1CC(C(=O)NC23CC(n4cnc(-c5ccc(F)c(F)c5)c4)(C2)C3)Oc2ccc(Cl)cc21] |
| 149 | 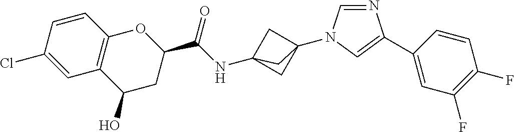 [SMILES: O=C(NC12CC(n3cnc(-c4ccc(F)c(F)c4)c3)(C1)C2)[C@H]1C[C@@H](O)c2cc(Cl)ccc2O1] |
| 150 | 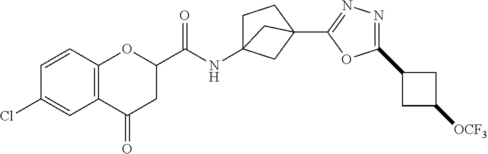 |
| 151 | 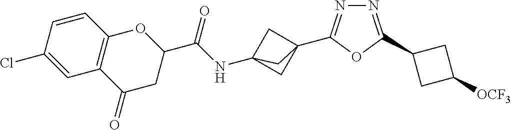 [SMILES: O=C1CC(C(=O)NC23CC(c4nnc([C@H]5C[C@@H](OC(F)(F)F)C5)o4)(C2)C3)Oc2ccc(Cl)cc21] |
| 152 | 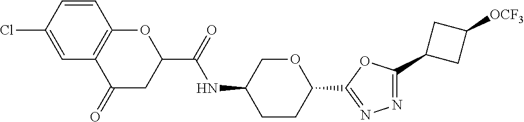 [SMILES: O=C1CC(C(=O)N[C@@H]2CC[C@@H](c3nnc([C@H]4C[C@@H](OC(F)(F)F)C4)o3)OC2)Oc2ccc(Cl)cc21] |
| 153 | 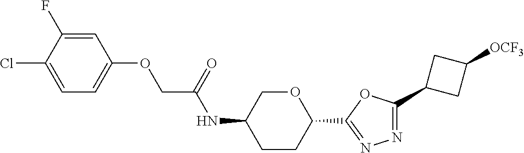 [SMILES: O=C(COc1ccc(Cl)c(F)c1)N[C@@H]1CC[C@@H](c2nnc([C@H]3C[C@@H](OC(F)(F)F)C3)o2)OC1] |
| 154 | 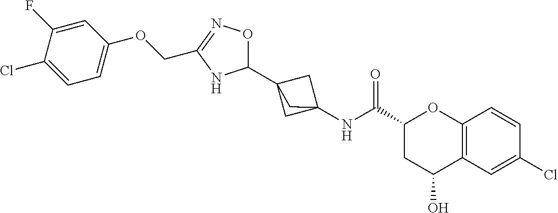 [SMILES: O=C(NC12CC(C3NC(COc4ccc(Cl)c(F)c4)=NO3)(C1)C2)[C@H]1C[C@@H](O)c2cc(Cl)ccc2O1] |
| 155 | 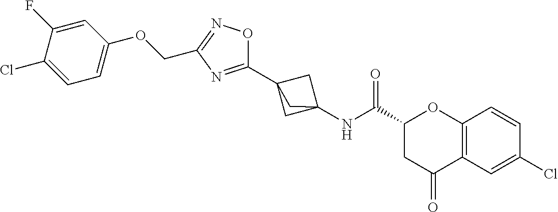 [SMILES: O=C1C[C@H](C(=O)NC23CC(c4nc(COc5ccc(Cl)c(F)c5)no4)(C2)C3)Oc2ccc(Cl)cc21] |
| 156 | 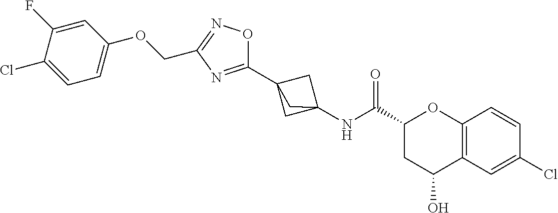 [SMILES: O=C(NC12CC(c3nc(COc4ccc(Cl)c(F)c4)no3)(C1)C2)[C@H]1C[C@@H](O)c2cc(Cl)ccc2O1] |
| 157 | 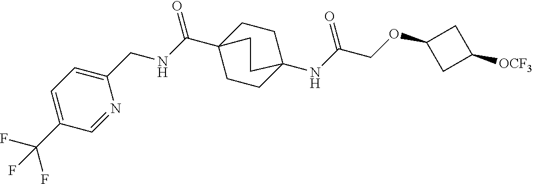 [SMILES: O=C(CO[C@H]1C[C@@H](OC(F)(F)F)C1)NC12CCC(C(=O)NCc3ccc(C(F)(F)F)cn3)(CC1)CC2] |
| 158 | 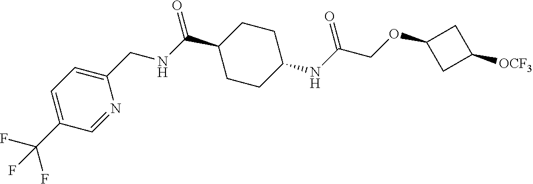 [SMILES: O=C(CO[C@H]1C[C@@H](OC(F)(F)F)C1)N[C@H]1CC[C@H](C(=O)NCc2ccc(C(F)(F)F)cn2)CC1] |
| 159 | 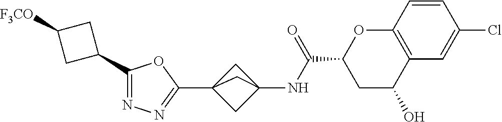 [SMILES: O=C(NC12CC(c3nnc([C@H]4C[C@@H](OC(F)(F)F)C4)o3)(C1)C2)[C@H]1C[C@@H](O)c2cc(Cl)ccc2O1] |
| 160 | 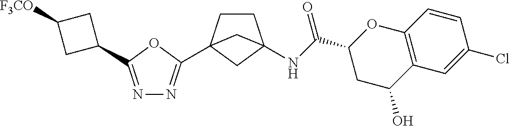 [SMILES: O=C(NC12CCC(c3nnc([C@H]4C[C@@H](OC(F)(F)F)C4)o3)(C1)C2)[C@H]1C[C@@H](O)c2cc(Cl)ccc2O1] |
| 161 | 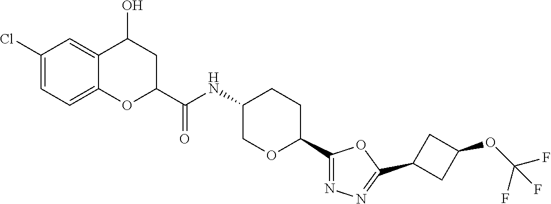 [SMILES: O=C(N[C@@H]1CC[C@@H](c2nnc([C@H]3C[C@@H](OC(F)(F)F)C3)o2)OC1)C1CC(O)c2cc(Cl)ccc2O1] |
| 162 | 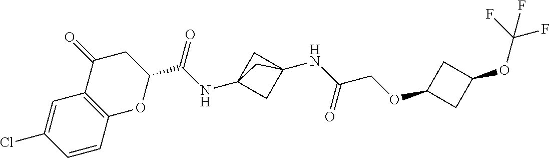 [SMILES: O=C(CO[C@H]1C[C@@H](OC(F)(F)F)C1)NC12CC(NC(=O)[C@H]3CC(=O)c4cc(Cl)ccc4O3)(C1)C2] |
| 163 | 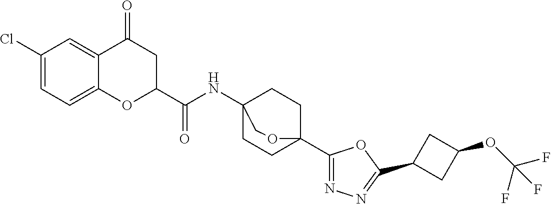 [SMILES: O=C1CC(C(=O)NC23CCC(c4nnc([C@H]5C[C@@H](OC(F)(F)F)C5)o4)(CC2)OC3)Oc2ccc(Cl)cc21] |
| 164 | 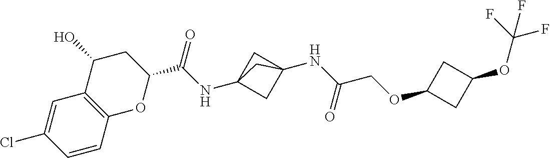 [SMILES: O=C(CO[C@H]1C[C@@H](OC(F)(F)F)C1)NC12CC(NC(=O)[C@H]3C[C@@H](O)c4cc(Cl)ccc4O3)(C1)C2] |
| 165 | 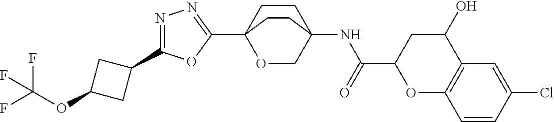 [SMILES: O=C(NC12CCC(c3nnc([C@H]4C[C@@H](OC(F)(F)F)C4)o3)(CC1)OC2)C1CC(O)c2cc(Cl)ccc2O1] |
| 166 | 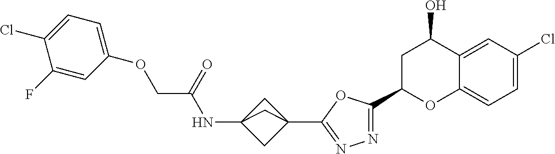 [SMILES: O=C(COc1ccc(Cl)c(F)c1)NC12CC(c3nnc([C@H]4C[C@@H](O)c5cc(Cl)ccc5O4)o3)(C1)C2] |
| 167 | 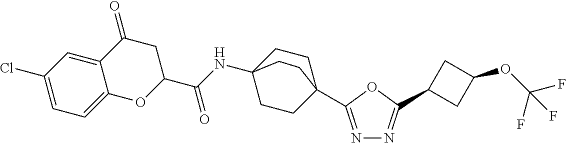 [SMILES: O=C1CC(C(=O)NC23CCC(c4nnc([C@H]5C[C@@H](OC(F)(F)F)C5)o4)(CC2)CC3)Oc2ccc(Cl)cc21] |
| 168 | 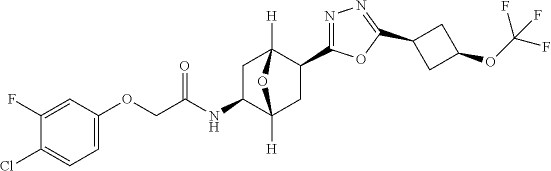 [SMILES: O=C(COc1ccc(Cl)c(F)c1)N[C@H]1C[C@H]2O[C@@H]1C[C@@H]2c1nnc([C@H]2C[C@@H](OC(F)(F)F)C2)o1] |
| 169 | 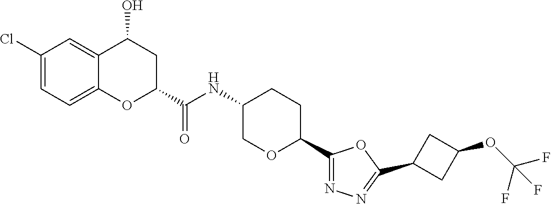 [SMILES: O=C(N[C@@H]1CC[C@@H](c2nnc([C@H]3C[C@@H](OC(F)(F)F)C3)o2)OC1)[C@H]1C[C@@H](O)c2cc(Cl)ccc2O1] |
| 170 | 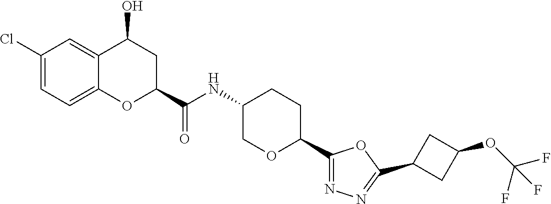 [SMILES: O=C(N[C@@H]1CC[C@@H](c2nnc([C@H]3C[C@@H](OC(F)(F)F)C3)o2)OC1)[C@@H]1C[C@H](O)c2cc(Cl)ccc2O1] |
| 171 | 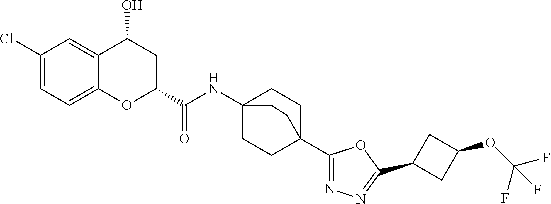 [SMILES: O=C(NC12CCC(c3nnc([C@H]4C[C@@H](OC(F)(F)F)C4)o3)(CC1)CC2)[C@H]1C[C@@H](O)c2cc(Cl)ccc2O1] |
| 172 | 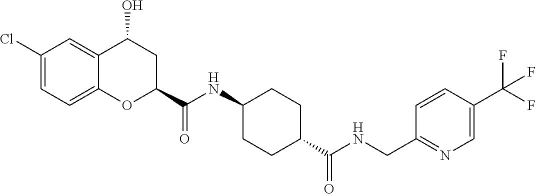 [SMILES: O=C(N[C@H]1CC[C@H](C(=O)NCc2ccc(C(F)(F)F)cn2)CC1)[C@@H]1C[C@@H](O)c2cc(Cl)ccc2O1] |
| 173 | 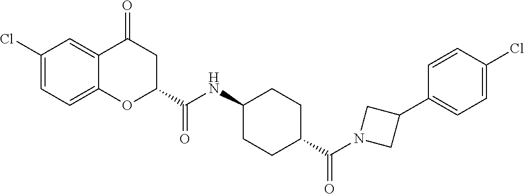 [SMILES: O=C1C[C@H](C(=O)N[C@H]2CC[C@H](C(=O)N3CC(c4ccc(Cl)cc4)C3)CC2)Oc2ccc(Cl)cc21] |
| 174 | 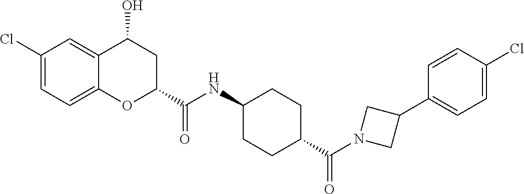 [SMILES: O=C(N[C@H]1CC[C@H](C(=O)N2CC(c3ccc(Cl)cc3)C2)CC1)[C@H]1C[C@@H](O)c2cc(Cl)ccc2O1] |
| 175 | 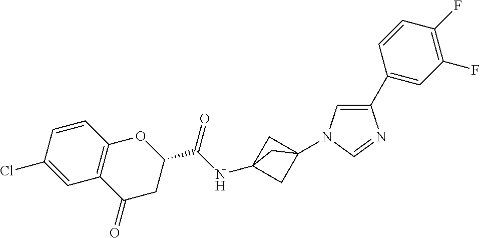 [SMILES: O=C1C[C@@H](C(=O)NC23CC(n4cnc(-c5ccc(F)c(F)c5)c4)(C2)C3)Oc2ccc(Cl)cc21] |
| 176 | 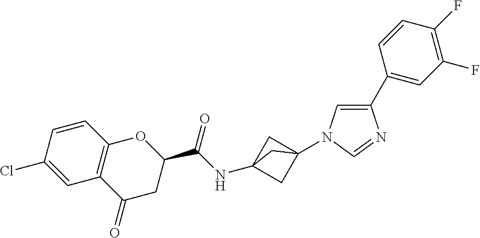 [SMILES: O=C1C[C@H](C(=O)NC23CC(n4cnc(-c5ccc(F)c(F)c5)c4)(C2)C3)Oc2ccc(Cl)cc21] |
| 177 | 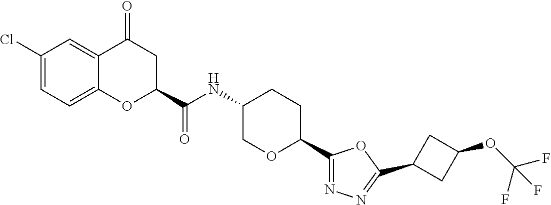 [SMILES: O=C1C[C@@H](C(=O)N[C@@H]2CC[C@@H](c3nnc([C@H]4C[C@@H](OC(F)(F)F)C4)o3)OC2)Oc2ccc(Cl)cc21] |
| 178 | 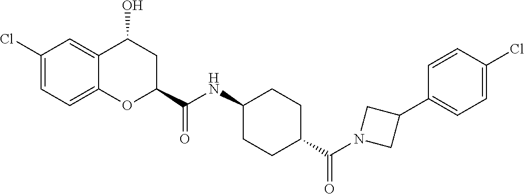 [SMILES: O=C(N[C@H]1CC[C@H](C(=O)N2CC(c3ccc(Cl)cc3)C2)CC1)[C@@H]1C[C@@H](O)c2cc(Cl)ccc2O1] |
| 179 | 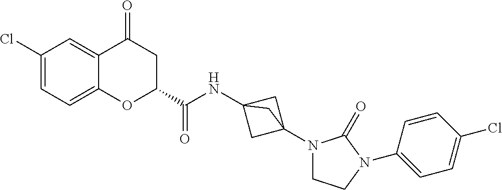 [SMILES: O=C1C[C@H](C(=O)NC23CC(N4CCN(c5ccc(Cl)cc5)C4=O)(C2)C3)Oc2ccc(Cl)cc21] |
| 180 | 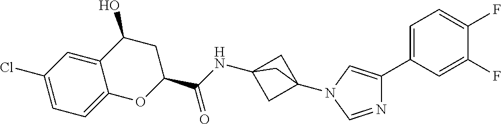 [SMILES: O=C(NC12CC(n3cnc(-c4ccc(F)c(F)c4)c3)(C1)C2)[C@@H]1C[C@H](O)c2cc(Cl)ccc2O1] |
| 181 | 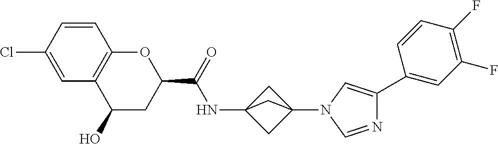 [SMILES: O=C(NC12CC(n3cnc(-c4ccc(F)c(F)c4)c3)(C1)C2)[C@H]1C[C@@H](O)c2cc(Cl)ccc2O1] |
| 182 | 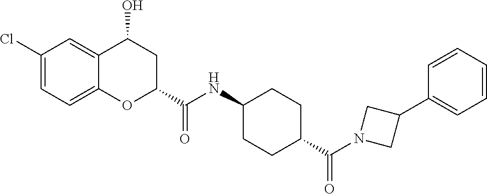 [SMILES: O=C(N[C@H]1CC[C@H](C(=O)N2CC(c3ccccc3)C2)CC1)[C@H]1C[C@@H](O)c2cc(Cl)ccc2O1] |
| 183 | 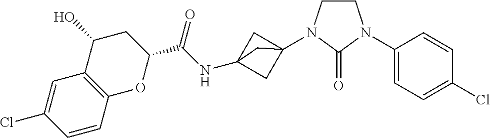 [SMILES: O=C(NC12CC(N3CCN(c4ccc(Cl)cc4)C3=O)(C1)C2)[C@H]1C[C@@H](O)c2cc(Cl)ccc2O1] |
| 184 | 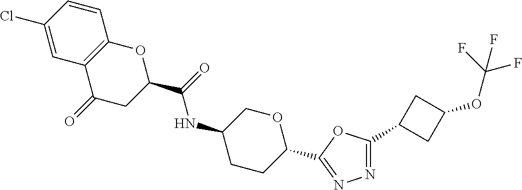 [SMILES: O=C1C[C@H](C(=O)N[C@@H]2CC[C@@H](c3nnc([C@H]4C[C@@H](OC(F)(F)F)C4)o3)OC2)Oc2ccc(Cl)cc21] |
| 185 | 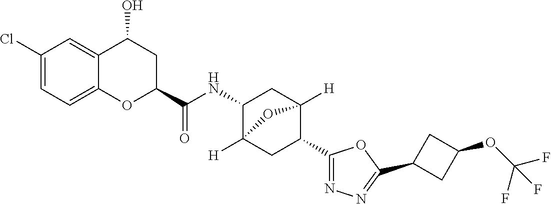 [SMILES: O=C(N[C@@H]1C[C@@H]2O[C@H]1C[C@H]2c1nnc([C@H]2C[C@@H](OC(F)(F)F)C2)o1)[C@@H]1C[C@@H](O)c2cc(Cl)ccc2O1] |
| 186 | 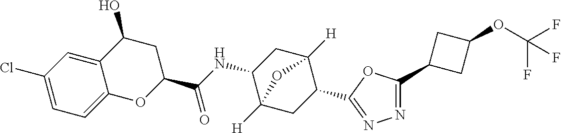 [SMILES: O=C(N[C@@H]1C[C@@H]2O[C@H]1C[C@H]2c1nnc([C@H]2C[C@@H](OC(F)(F)F)C2)o1)[C@@H]1C[C@H](O)c2cc(Cl)ccc2O1] |
| 187 | 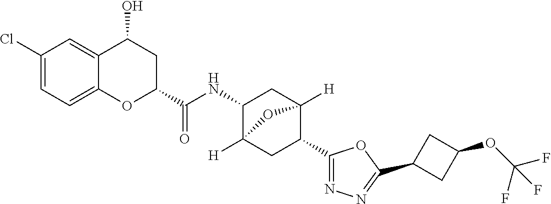 [SMILES: O=C(N[C@@H]1C[C@@H]2O[C@H]1C[C@H]2c1nnc([C@H]2C[C@@H](OC(F)(F)F)C2)o1)[C@H]1C[C@@H](O)c2cc(Cl)ccc2O1] |
| 188 | 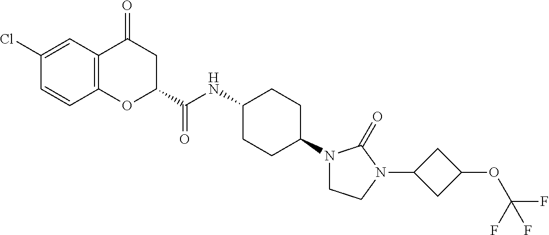 [SMILES: O=C1C[C@H](C(=O)N[C@H]2CC[C@H](N3CCN(C4CC(OC(F)(F)F)C4)C3=O)CC2)Oc2ccc(Cl)cc21] |
| 189 | 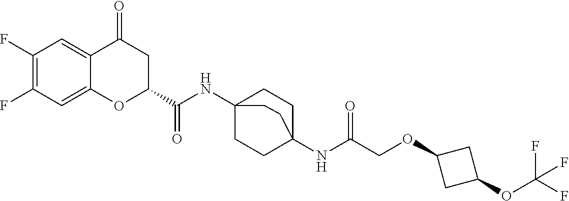 [SMILES: O=C(CO[C@H]1C[C@@H](OC(F)(F)F)C1)NC12CCC(NC(=O)[C@H]3CC(=O)c4cc(F)c(F)cc4O3)(CC1)CC2] |
| 190 | 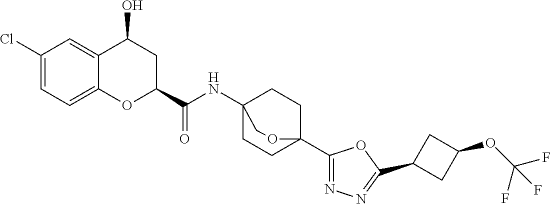 [SMILES: O=C(NC12CCC(c3nnc([C@H]4C[C@@H](OC(F)(F)F)C4)o3)(CC1)OC2)[C@@H]1C[C@H](O)c2cc(Cl)ccc2O1] |
| 191 | 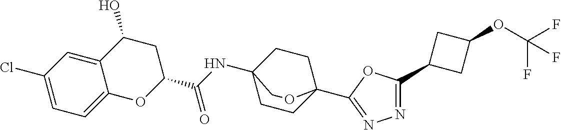 [SMILES: O=C(NC12CCC(c3nnc([C@H]4C[C@@H](OC(F)(F)F)C4)o3)(CC1)OC2)[C@H]1C[C@@H](O)c2cc(Cl)ccc2O1] |
| 192 | 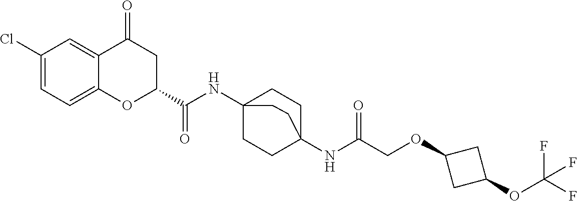 [SMILES: O=C(CO[C@H]1C[C@@H](OC(F)(F)F)C1)NC12CCC(NC(=O)[C@H]3CC(=O)c4cc(Cl)ccc4O3)(CC1)CC2] |
| 193 | 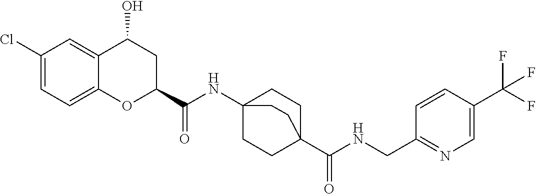 [SMILES: O=C(NC12CCC(C(=O)NCc3ccc(C(F)(F)F)cn3)(CC1)CC2)[C@@H]1C[C@@H](O)c2cc(Cl)ccc2O1] |
| 194 | 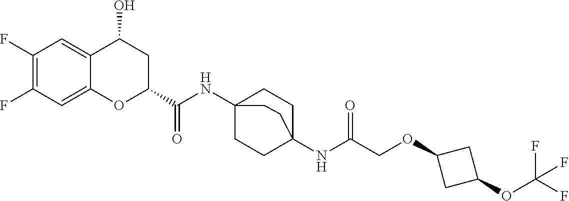 [SMILES: O=C(CO[C@H]1C[C@@H](OC(F)(F)F)C1)NC12CCC(NC(=O)[C@H]3C[C@@H](O)c4cc(F)c(F)cc4O3)(CC1)CC2] |
| 195 | 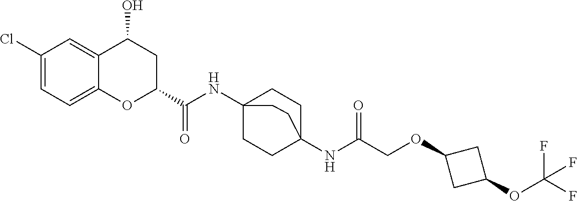 [SMILES: O=C(CO[C@H]1C[C@@H](OC(F)(F)F)C1)NC12CCC(NC(=O)[C@H]3C[C@@H](O)c4cc(Cl)ccc4O3)(CC1)CC2] |
| 196 | 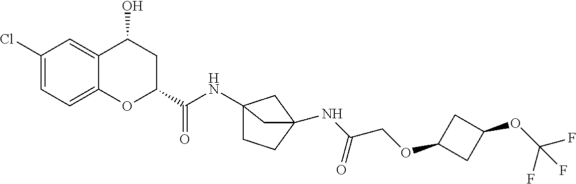 [SMILES: O=C(CO[C@H]1C[C@@H](OC(F)(F)F)C1)NC12CCC(NC(=O)[C@H]3C[C@@H](O)c4cc(Cl)ccc4O3)(C1)C2] |
| 197 | 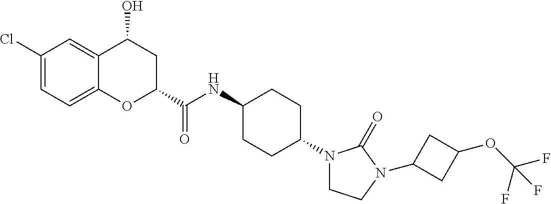 [SMILES: O=C(N[C@H]1CC[C@H](N2CCN(C3CC(OC(F)(F)F)C3)C2=O)CC1)[C@H]1C[C@@H](O)c2cc(Cl)ccc2O1] |
| 198 | 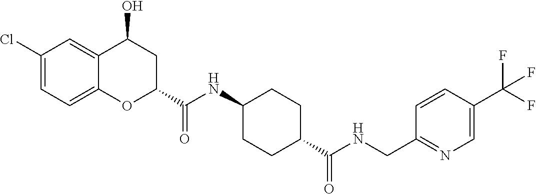 [SMILES: O=C(N[C@H]1CC[C@H](C(=O)NCc2ccc(C(F)(F)F)cn2)CC1)[C@H]1C[C@H](O)c2cc(Cl)ccc2O1] |
| 199 | 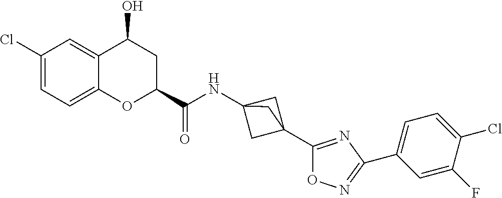 [SMILES: O=C(NC12CC(c3nc(-c4ccc(Cl)c(F)c4)no3)(C1)C2)[C@@H]1C[C@H](O)c2cc(Cl)ccc2O1] |
| 200 | 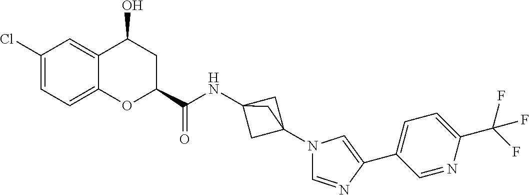 [SMILES: O=C(NC12CC(n3cnc(-c4ccc(C(F)(F)F)nc4)c3)(C1)C2)[C@@H]1C[C@H](O)c2cc(Cl)ccc2O1] |
| 201 | 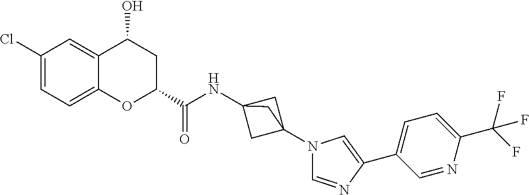 [SMILES: O=C(NC12CC(n3cnc(-c4ccc(C(F)(F)F)nc4)c3)(C1)C2)[C@H]1C[C@@H](O)c2cc(Cl)ccc2O1] |
| 202 | 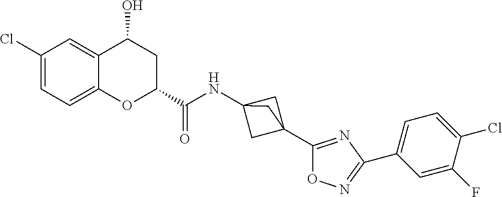 [SMILES: O=C(NC12CC(c3nc(-c4ccc(Cl)c(F)c4)no3)(C1)C2)[C@H]1C[C@@H](O)c2cc(Cl)ccc2O1] |
| 203 | 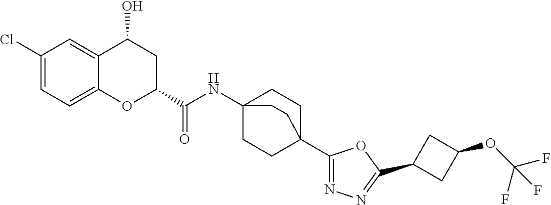 [SMILES: O=C(NC12CCC(c3nnc([C@H]4C[C@@H](OC(F)(F)F)C4)o3)(CC1)CC2)[C@H]1C[C@@H](O)c2cc(Cl)ccc2O1] |
| 204 | 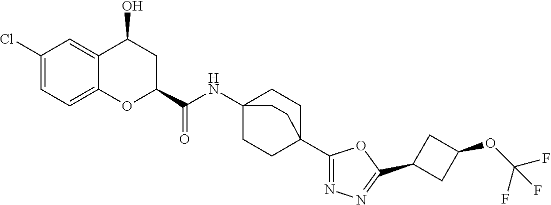 [SMILES: O=C(NC12CCC(c3nnc([C@H]4C[C@@H](OC(F)(F)F)C4)o3)(CC1)CC2)[C@@H]1C[C@H](O)c2cc(Cl)ccc2O1] |
| 205 | 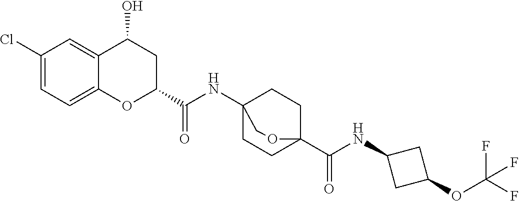 [SMILES: O=C(NC12CCC(C(=O)N[C@H]3C[C@@H](OC(F)(F)F)C3)(CC1)OC2)[C@H]1C[C@@H](O)c2cc(Cl)ccc2O1] |
| 206 | 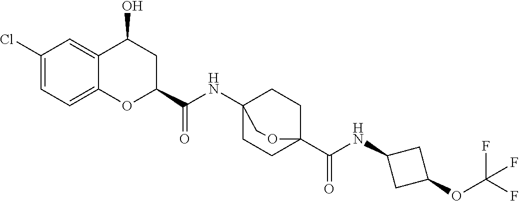 [SMILES: O=C(NC12CCC(C(=O)N[C@H]3C[C@@H](OC(F)(F)F)C3)(CC1)OC2)[C@@H]1C[C@H](O)c2cc(Cl)ccc2O1] |
| 207 | 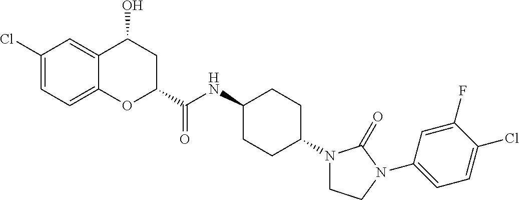 [SMILES: O=C(N[C@H]1CC[C@H](N2CCN(c3ccc(Cl)c(F)c3)C2=O)CC1)[C@H]1C[C@@H](O)c2cc(Cl)ccc2O1] |
| 208 | 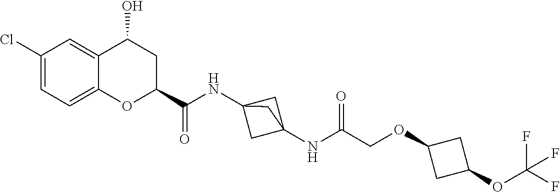 [SMILES: O=C(CO[C@H]1C[C@@H](OC(F)(F)F)C1)NC12CC(NC(=O)[C@@H]3C[C@@H](O)c4cc(Cl)ccc4O3)(C1)C2] |
| 209 | 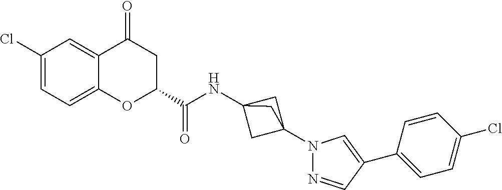 [SMILES: O=C1C[C@H](C(=O)NC23CC(n4cc(-c5ccc(Cl)cc5)cn4)(C2)C3)Oc2ccc(Cl)cc21] |
| 210 | 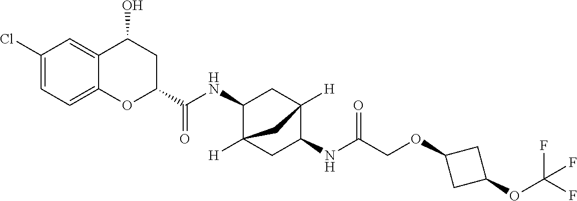 [SMILES: O=C(CO[C@H]1C[C@@H](OC(F)(F)F)C1)N[C@H]1C[C@H]2C[C@@H]1C[C@@H]2NC(=O)[C@H]1C[C@@H](O)c2cc(Cl)ccc2O1] |
| 211 | 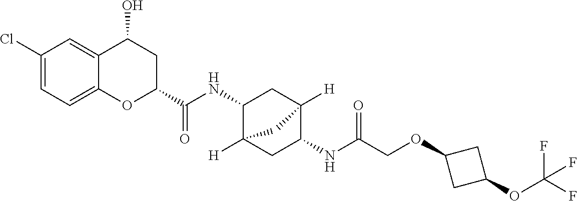 [SMILES: O=C(CO[C@H]1C[C@@H](OC(F)(F)F)C1)N[C@@H]1C[C@@H]2C[C@H]1C[C@H]2NC(=O)[C@H]1C[C@@H](O)c2cc(Cl)ccc2O1] |
| 212 | 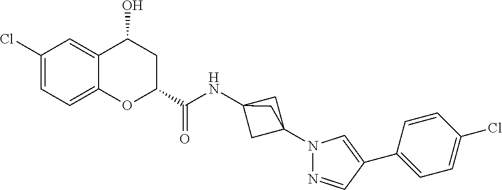 [SMILES: O=C(NC12CC(n3cc(-c4ccc(Cl)cc4)cn3)(C1)C2)[C@H]1C[C@@H](O)c2cc(Cl)ccc2O1] |
| 213 | 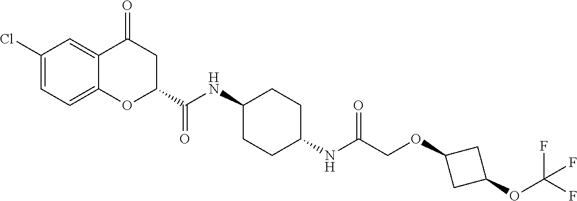 [SMILES: O=C(CO[C@H]1C[C@@H](OC(F)(F)F)C1)N[C@H]1CC[C@H](NC(=O)[C@H]2CC(=O)c3cc(Cl)ccc3O2)CC1] |
| 214 | 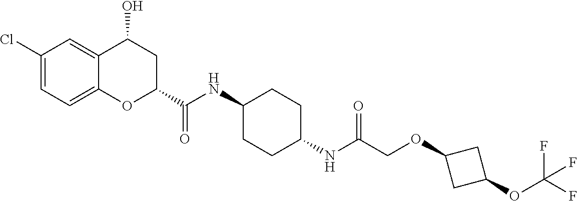 [SMILES: O=C(CO[C@H]1C[C@@H](OC(F)(F)F)C1)N[C@H]1CC[C@H](NC(=O)[C@H]2C[C@@H](O)c3cc(Cl)ccc3O2)CC1] |
| 215 | 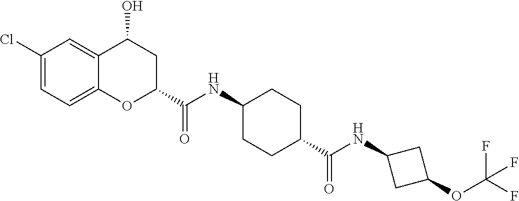 [SMILES: O=C(N[C@H]1CC[C@H](C(=O)N[C@H]2C[C@@H](OC(F)(F)F)C2)CC1)[C@H]1C[C@@H](O)c2cc(Cl)ccc2O1] |
| 216 | 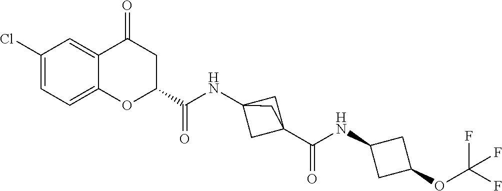 [SMILES: O=C1C[C@H](C(=O)NC23CC(C(=O)N[C@H]4C[C@@H](OC(F)(F)F)C4)(C2)C3)Oc2ccc(Cl)cc21] |
| 217 | 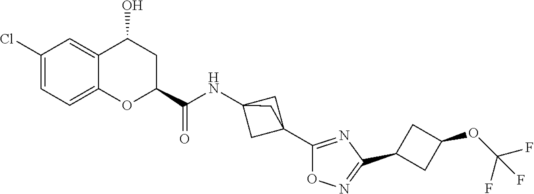 [SMILES: O=C(NC12CC(c3nc([C@H]4C[C@@H](OC(F)(F)F)C4)no3)(C1)C2)[C@@H]1C[C@@H](O)c2cc(Cl)ccc2O1] |
| 218 | 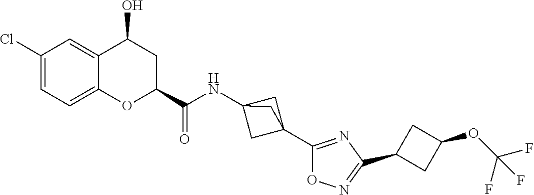 [SMILES: O=C(NC12CC(c3nc([C@H]4C[C@@H](OC(F)(F)F)C4)no3)(C1)C2)[C@@H]1C[C@H](O)c2cc(Cl)ccc2O1] |
| 219 | 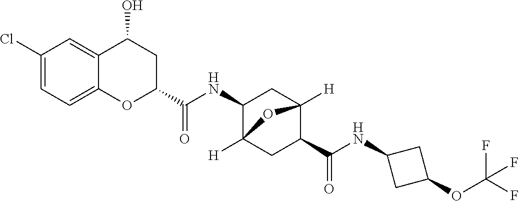 [SMILES: O=C(N[C@H]1C[C@@H](OC(F)(F)F)C1)[C@H]1C[C@H]2O[C@@H]1C[C@@H]2NC(=O)[C@H]1C[C@@H](O)c2cc(Cl)ccc2O1] |
| 220 | 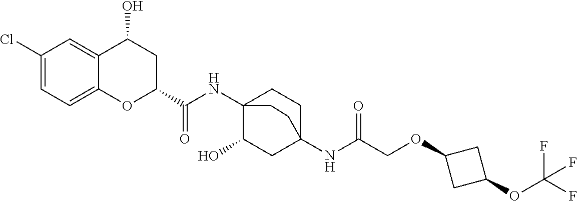 [SMILES: O=C(CO[C@H]1C[C@@H](OC(F)(F)F)C1)NC12CCC(NC(=O)[C@H]3C[C@@H](O)c4cc(Cl)ccc4O3)(CC1)[C@@H](O)C2] |
| 221 | 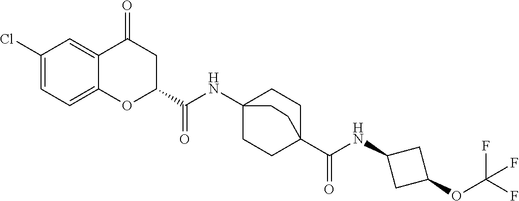 [SMILES: O=C1C[C@H](C(=O)NC23CCC(C(=O)N[C@H]4C[C@@H](OC(F)(F)F)C4)(CC2)CC3)Oc2ccc(Cl)cc21] |
| 222 | 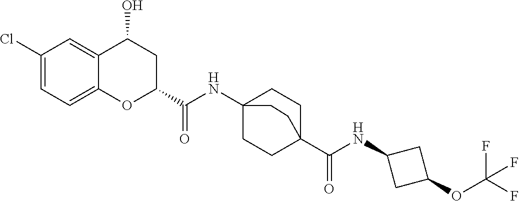 [SMILES: O=C(NC12CCC(C(=O)N[C@H]3C[C@@H](OC(F)(F)F)C3)(CC1)CC2)[C@H]1C[C@@H](O)c2cc(Cl)ccc2O1] |
| 223 | 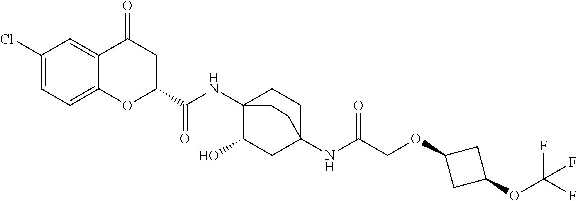 [SMILES: O=C(CO[C@H]1C[C@@H](OC(F)(F)F)C1)NC12CCC(NC(=O)[C@H]3CC(=O)c4cc(Cl)ccc4O3)(CC1)[C@@H](O)C2] |
| 224 | 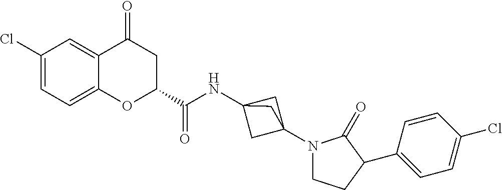 [SMILES: O=C1C[C@H](C(=O)NC23CC(N4CCC(c5ccc(Cl)cc5)C4=O)(C2)C3)Oc2ccc(Cl)cc21] |
| 225 | 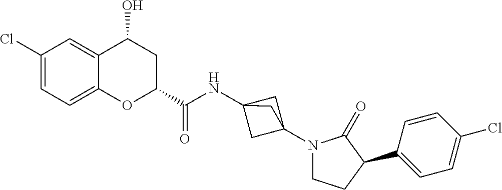 [SMILES: O=C(NC12CC(N3CC[C@H](c4ccc(Cl)cc4)C3=O)(C1)C2)[C@H]1C[C@@H](O)c2cc(Cl)ccc2O1] |
| 226 | 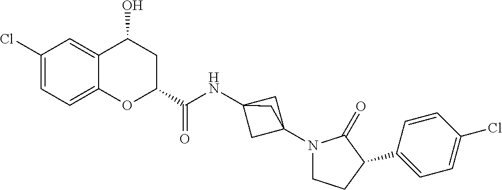 [SMILES: O=C(NC12CC(N3CC[C@@H](c4ccc(Cl)cc4)C3=O)(C1)C2)[C@H]1C[C@@H](O)c2cc(Cl)ccc2O1] |
| 227 | 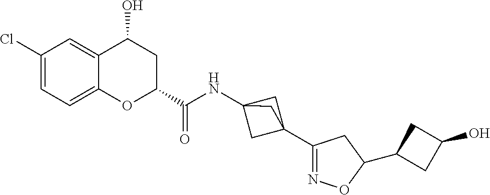 [SMILES: O=C(NC12CC(C3=NOC([C@H]4C[C@@H](O)C4)C3)(C1)C2)[C@H]1C[C@@H](O)c2cc(Cl)ccc2O1] |
| 228 | 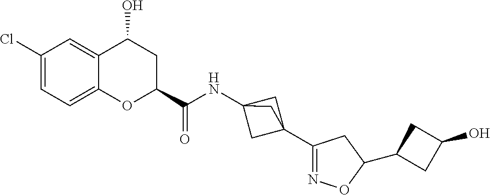 [SMILES: O=C(NC12CC(C3=NOC([C@H]4C[C@@H](O)C4)C3)(C1)C2)[C@@H]1C[C@@H](O)c2cc(Cl)ccc2O1] |
| 229 | 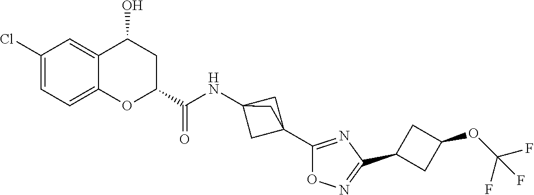 [SMILES: O=C(NC12CC(c3nc([C@H]4C[C@@H](OC(F)(F)F)C4)no3)(C1)C2)[C@H]1C[C@@H](O)c2cc(Cl)ccc2O1] |
| 230 | 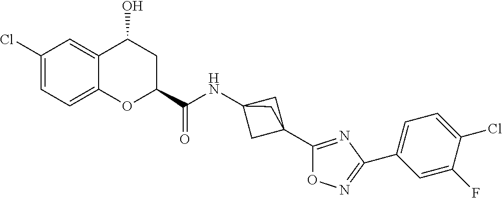 [SMILES: O=C(NC12CC(c3nc(-c4ccc(Cl)c(F)c4)no3)(C1)C2)[C@@H]1C[C@@H](O)c2cc(Cl)ccc2O1] |
| 231 | 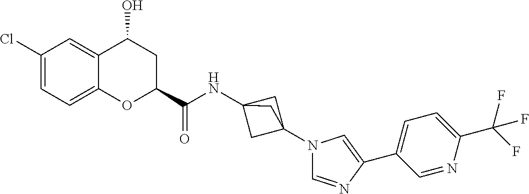 [SMILES: O=C(NC12CC(n3cnc(-c4ccc(C(F)(F)F)nc4)c3)(C1)C2)[C@@H]1C[C@@H](O)c2cc(Cl)ccc2O1] |
| 232 | 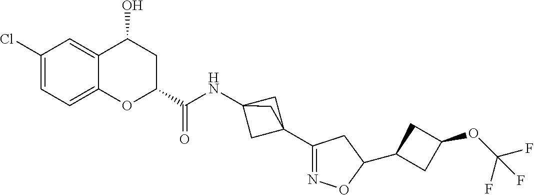 [SMILES: O=C(NC12CC(C3=NOC([C@H]4C[C@@H](OC(F)(F)F)C4)C3)(C1)C2)[C@H]1C[C@@H](O)c2cc(Cl)ccc2O1] |
| 233 | 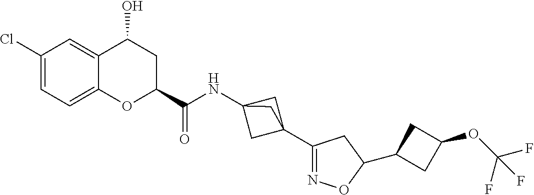 [SMILES: O=C(NC12CC(C3=NOC([C@H]4C[C@@H](OC(F)(F)F)C4)C3)(C1)C2)[C@@H]1C[C@@H](O)c2cc(Cl)ccc2O1] |
| 234 | 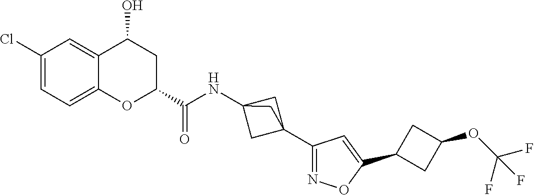 [SMILES: O=C(NC12CC(c3cc([C@H]4C[C@@H](OC(F)(F)F)C4)on3)(C1)C2)[C@H]1C[C@@H](O)c2cc(Cl)ccc2O1] |
| 235 | 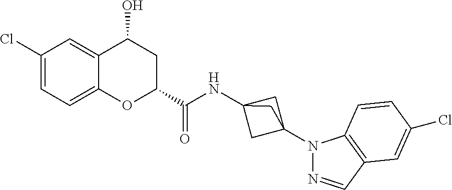 [SMILES: O=C(NC12CC(n3ncc4cc(Cl)ccc43)(C1)C2)[C@H]1C[C@@H](O)c2cc(Cl)ccc2O1] |
| 236 | 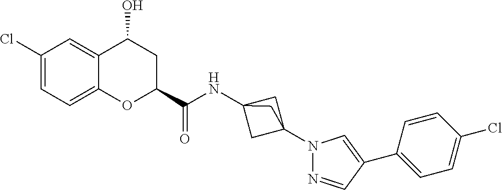 [SMILES: O=C(NC12CC(n3cc(-c4ccc(Cl)cc4)cn3)(C1)C2)[C@@H]1C[C@@H](O)c2cc(Cl)ccc2O1] |
| 237 | 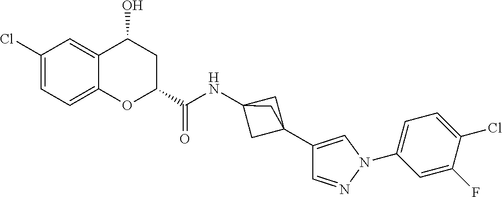 [SMILES: O=C(NC12CC(c3cnn(-c4ccc(Cl)c(F)c4)c3)(C1)C2)[C@H]1C[C@@H](O)c2cc(Cl)ccc2O1] |
| 238 | 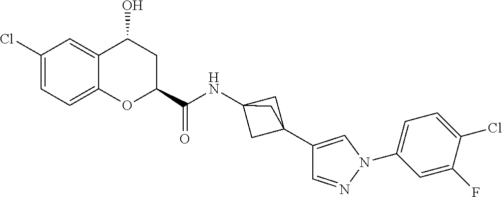 [SMILES: O=C(NC12CC(c3cnn(-c4ccc(Cl)c(F)c4)c3)(C1)C2)[C@@H]1C[C@@H](O)c2cc(Cl)ccc2O1] |
| 239 | 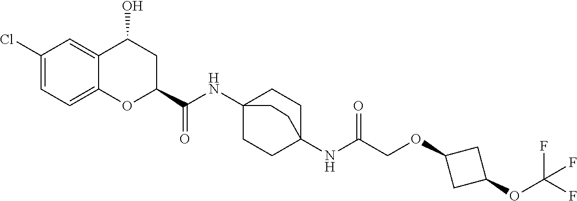 [SMILES: O=C(CO[C@H]1C[C@@H](OC(F)(F)F)C1)NC12CCC(NC(=O)[C@@H]3C[C@@H](O)c4cc(Cl)ccc4O3)(CC1)CC2] |
| 240 | 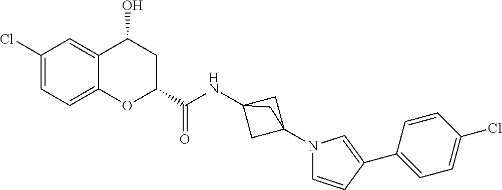 [SMILES: O=C(NC12CC(n3ccc(-c4ccc(Cl)cc4)c3)(C1)C2)[C@H]1C[C@@H](O)c2cc(Cl)ccc2O1] |
| 241 | 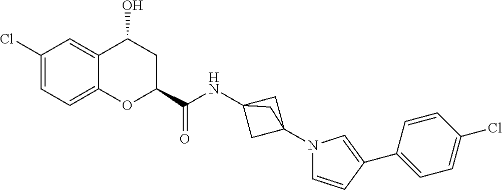 [SMILES: O=C(NC12CC(n3ccc(-c4ccc(Cl)cc4)c3)(C1)C2)[C@@H]1C[C@@H](O)c2cc(Cl)ccc2O1] |
| 242 | 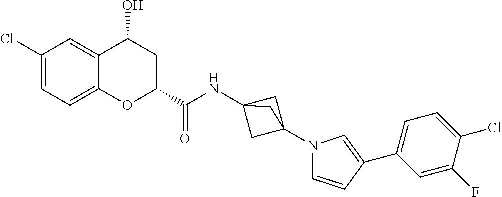 [SMILES: O=C(NC12CC(n3ccc(-c4ccc(Cl)c(F)c4)c3)(C1)C2)[C@H]1C[C@@H](O)c2cc(Cl)ccc2O1] |
| 243 | 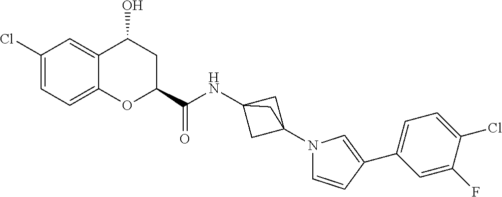 [SMILES: O=C(NC12CC(n3ccc(-c4ccc(Cl)c(F)c4)c3)(C1)C2)[C@@H]1C[C@@H](O)c2cc(Cl)ccc2O1] |
| 244 | 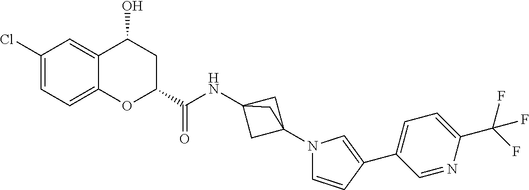 [SMILES: O=C(NC12CC(n3ccc(-c4ccc(C(F)(F)F)nc4)c3)(C1)C2)[C@H]1C[C@@H](O)c2cc(Cl)ccc2O1] |
| 245 | 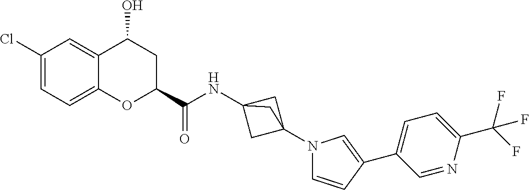 [SMILES: O=C(NC12CC(n3ccc(-c4ccc(C(F)(F)F)nc4)c3)(C1)C2)[C@@H]1C[C@@H](O)c2cc(Cl)ccc2O1] |
| 246 | 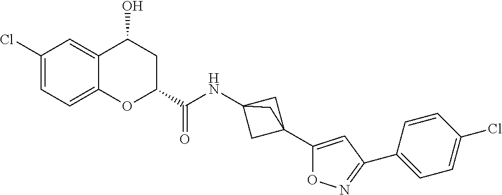 [SMILES: O=C(NC12CC(c3cc(-c4ccc(Cl)cc4)no3)(C1)C2)[C@H]1C[C@@H](O)c2cc(Cl)ccc2O1] |
| 247 | 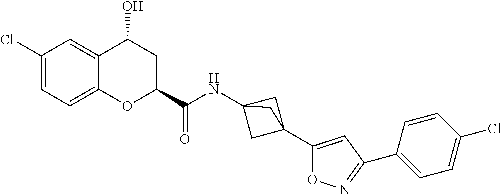 [SMILES: O=C(NC12CC(c3cc(-c4ccc(Cl)cc4)no3)(C1)C2)[C@@H]1C[C@@H](O)c2cc(Cl)ccc2O1] |
| 248 | 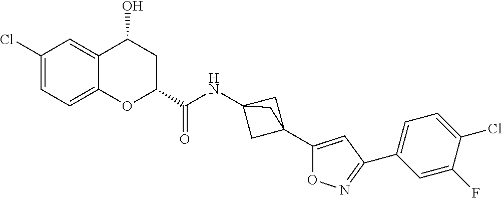 [SMILES: O=C(NC12CC(c3cc(-c4ccc(Cl)c(F)c4)no3)(C1)C2)[C@H]1C[C@@H](O)c2cc(Cl)ccc2O1] |
| 249 | 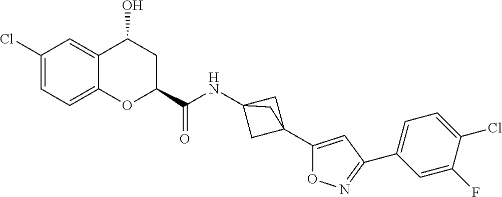 [SMILES: O=C(NC12CC(c3cc(-c4ccc(Cl)c(F)c4)no3)(C1)C2)[C@@H]1C[C@@H](O)c2cc(Cl)ccc2O1] |
| 250 | 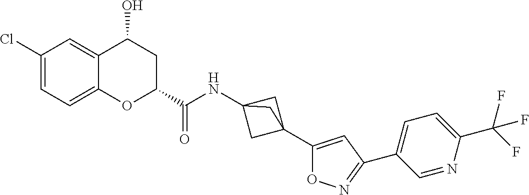 [SMILES: O=C(NC12CC(c3cc(-c4ccc(C(F)(F)F)nc4)no3)(C1)C2)[C@H]1C[C@@H](O)c2cc(Cl)ccc2O1] |
| 251 | 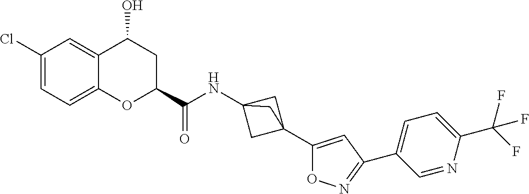 [SMILES: O=C(NC12CC(c3cc(-c4ccc(C(F)(F)F)nc4)no3)(C1)C2)[C@@H]1C[C@@H](O)c2cc(Cl)ccc2O1] |
| 252 | 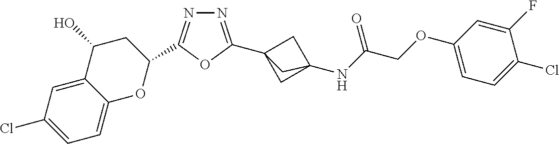 [SMILES: O=C(COc1ccc(Cl)c(F)c1)NC12CC(c3nnc([C@H]4C[C@@H](O)c5cc(Cl)ccc5O4)o3)(C1)C2] |
| 253 | 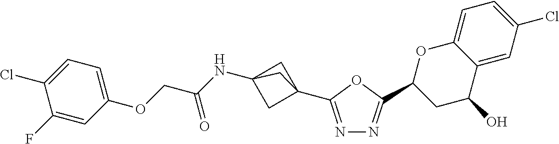 [SMILES: O=C(COc1ccc(Cl)c(F)c1)NC12CC(c3nnc([C@@H]4C[C@H](O)c5cc(Cl)ccc5O4)o3)(C1)C2] |
| 254 | 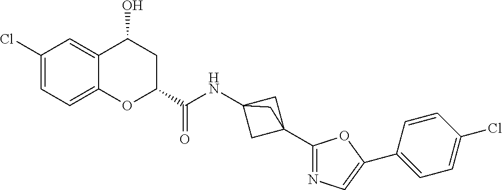 [SMILES: O=C(NC12CC(c3ncc(-c4ccc(Cl)cc4)o3)(C1)C2)[C@H]1C[C@@H](O)c2cc(Cl)ccc2O1] |
| 255 | 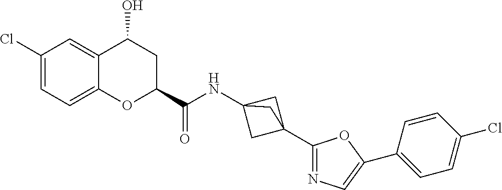 [SMILES: O=C(NC12CC(c3ncc(-c4ccc(Cl)cc4)o3)(C1)C2)[C@@H]1C[C@@H](O)c2cc(Cl)ccc2O1] |
| 256 | 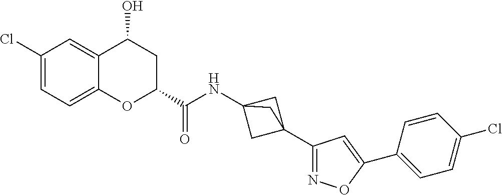 [SMILES: O=C(NC12CC(c3cc(-c4ccc(Cl)cc4)on3)(C1)C2)[C@H]1C[C@@H](O)c2cc(Cl)ccc2O1] |
| 257 | 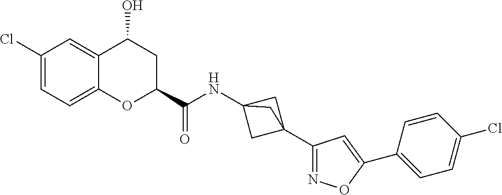 [SMILES: O=C(NC12CC(c3cc(-c4ccc(Cl)cc4)on3)(C1)C2)[C@@H]1C[C@@H](O)c2cc(Cl)ccc2O1] |
| 258 | 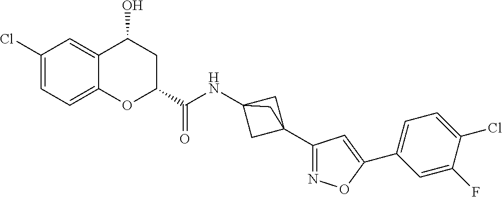 [SMILES: O=C(NC12CC(c3cc(-c4ccc(Cl)c(F)c4)on3)(C1)C2)[C@H]1C[C@@H](O)c2cc(Cl)ccc2O1] |
| 259 | 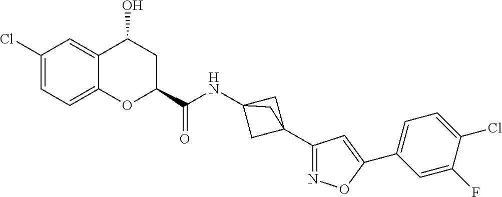 [SMILES: O=C(NC12CC(c3cc(-c4ccc(Cl)c(F)c4)on3)(C1)C2)[C@@H]1C[C@@H](O)c2cc(Cl)ccc2O1] |
| 260 | 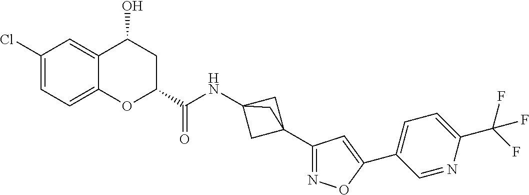 [SMILES: O=C(NC12CC(c3cc(-c4ccc(C(F)(F)F)nc4)on3)(C1)C2)[C@H]1C[C@@H](O)c2cc(Cl)ccc2O1] |
| 261 | 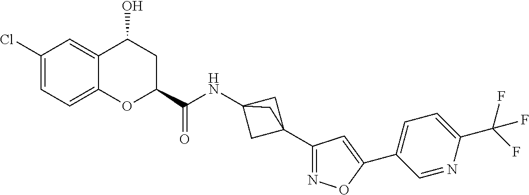 [SMILES: O=C(NC12CC(c3cc(-c4ccc(C(F)(F)F)nc4)on3)(C1)C2)[C@@H]1C[C@@H](O)c2cc(Cl)ccc2O1] |
| 262 | 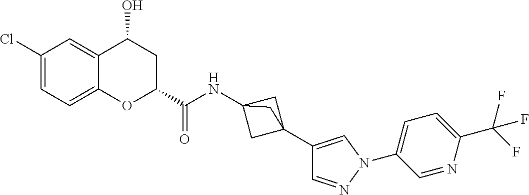 [SMILES: O=C(NC12CC(c3cnn(-c4ccc(C(F)(F)F)nc4)c3)(C1)C2)[C@H]1C[C@@H](O)c2cc(Cl)ccc2O1] |
| 263 | 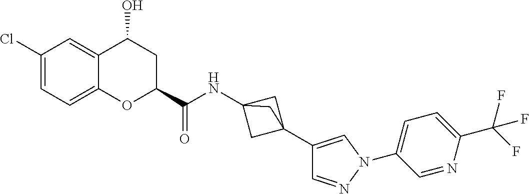 [SMILES: O=C(NC12CC(c3cnn(-c4ccc(C(F)(F)F)nc4)c3)(C1)C2)[C@@H]1C[C@@H](O)c2cc(Cl)ccc2O1] |
| 264 | 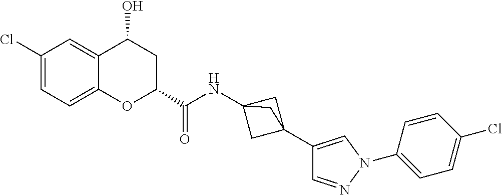 [SMILES: O=C(NC12CC(c3cnn(-c4ccc(Cl)cc4)c3)(C1)C2)[C@H]1C[C@@H](O)c2cc(Cl)ccc2O1] |
| 265 | 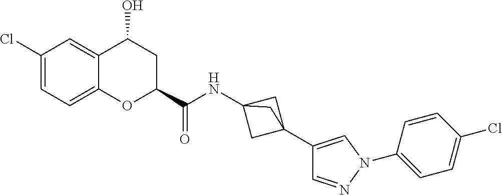 [SMILES: O=C(NC12CC(c3cnn(-c4ccc(Cl)cc4)c3)(C1)C2)[C@@H]1C[C@@H](O)c2cc(Cl)ccc2O1] |
| 266 | 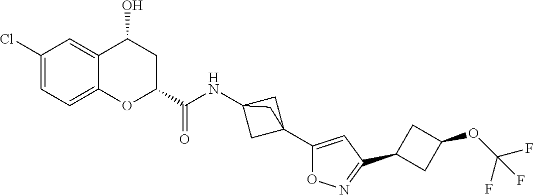 [SMILES: O=C(NC12CC(c3cc([C@H]4C[C@@H](OC(F)(F)F)C4)no3)(C1)C2)[C@H]1C[C@@H](O)c2cc(Cl)ccc2O1] |
| 267 | 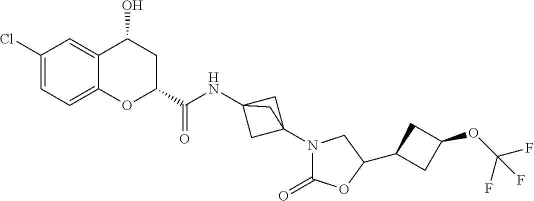 [SMILES: O=C(NC12CC(N3CC([C@H]4C[C@@H](OC(F)(F)F)C4)OC3=O)(C1)C2)[C@H]1C[C@@H](O)c2cc(Cl)ccc2O1] |
| 268 | 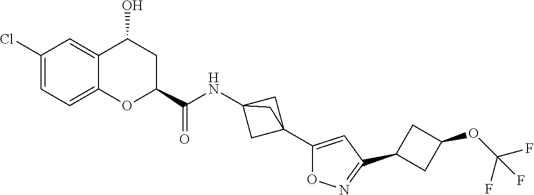 [SMILES: O=C(NC12CC(c3cc([C@H]4C[C@@H](OC(F)(F)F)C4)no3)(C1)C2)[C@@H]1C[C@@H](O)c2cc(Cl)ccc2O1] |
| 269 | 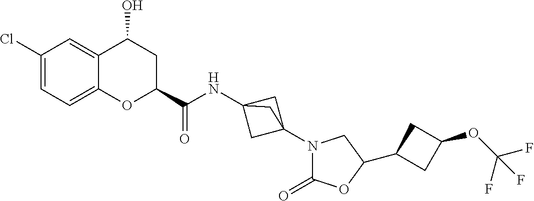 [SMILES: O=C(NC12CC(N3CC([C@H]4C[C@@H](OC(F)(F)F)C4)OC3=O)(C1)C2)[C@@H]1C[C@@H](O)c2cc(Cl)ccc2O1] |
| 270 | 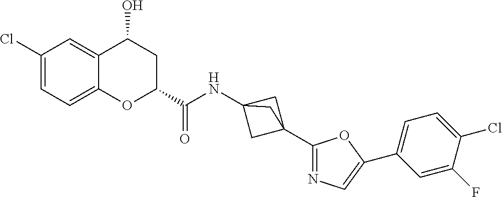 [SMILES: O=C(NC12CC(c3ncc(-c4ccc(Cl)c(F)c4)o3)(C1)C2)[C@H]1C[C@@H](O)c2cc(Cl)ccc2O1] |
| 271 | 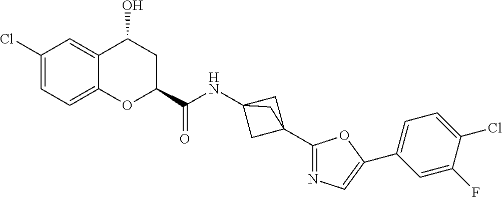 [SMILES: O=C(NC12CC(c3ncc(-c4ccc(Cl)c(F)c4)o3)(C1)C2)[C@@H]1C[C@@H](O)c2cc(Cl)ccc2O1] |
| 272 | 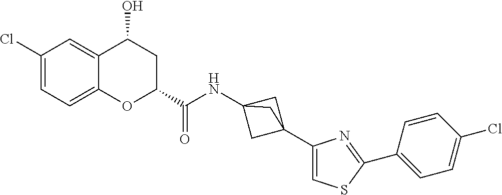 [SMILES: O=C(NC12CC(c3csc(-c4ccc(Cl)cc4)n3)(C1)C2)[C@H]1C[C@@H](O)c2cc(Cl)ccc2O1] |
| 273 | 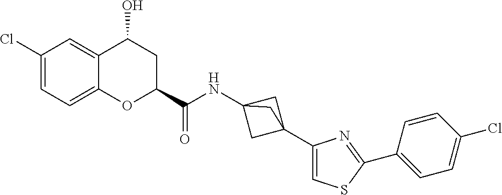 [SMILES: O=C(NC12CC(c3csc(-c4ccc(Cl)cc4)n3)(C1)C2)[C@@H]1C[C@@H](O)c2cc(Cl)ccc2O1] |
| 274 | 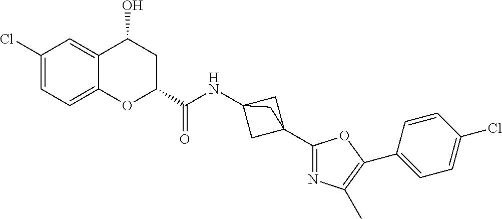 [SMILES: Cc1nc(C23CC(NC(=O)[C@H]4C[C@@H](O)c5cc(Cl)ccc5O4)(C2)C3)oc1-c1ccc(Cl)cc1] |
| 275 | 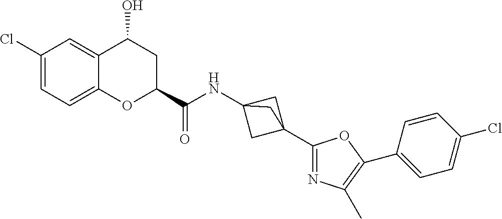 [SMILES: Cc1nc(C23CC(NC(=O)[C@@H]4C[C@@H](O)c5cc(Cl)ccc5O4)(C2)C3)oc1-c1ccc(Cl)cc1] |
| 276 | 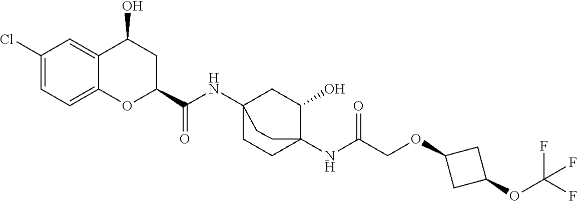 [SMILES: O=C(CO[C@H]1C[C@@H](OC(F)(F)F)C1)NC12CCC(NC(=O)[C@@H]3C[C@H](O)c4cc(Cl)ccc4O3)(CC1)C[C@@H]2O] |
| 277 | 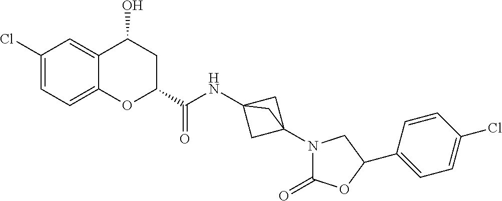 [SMILES: O=C(NC12CC(N3CC(c4ccc(Cl)cc4)OC3=O)(C1)C2)[C@H]1C[C@@H](O)c2cc(Cl)ccc2O1] |
| 278 | 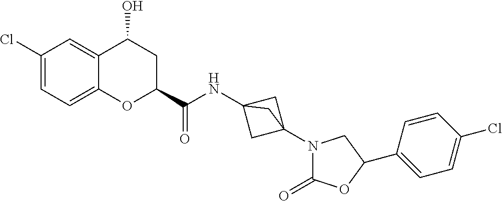 [SMILES: O=C(NC12CC(N3CC(c4ccc(Cl)cc4)OC3=O)(C1)C2)[C@@H]1C[C@@H](O)c2cc(Cl)ccc2O1] |
| 279 | 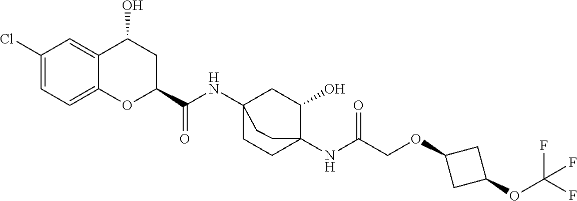 [SMILES: O=C(CO[C@H]1C[C@@H](OC(F)(F)F)C1)NC12CCC(NC(=O)[C@@H]3C[C@@H](O)c4cc(Cl)ccc4O3)(CC1)C[C@@H]2O] |
| 280 | 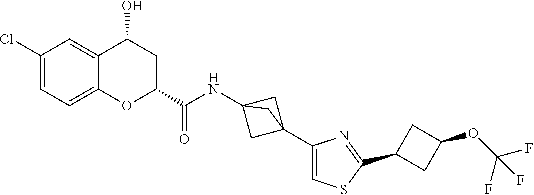 [SMILES: O=C(NC12CC(c3csc([C@H]4C[C@@H](OC(F)(F)F)C4)n3)(C1)C2)[C@H]1C[C@@H](O)c2cc(Cl)ccc2O1] |
| 281 | 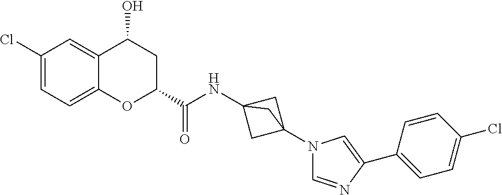 [SMILES: O=C(NC12CC(n3cnc(-c4ccc(Cl)cc4)c3)(C1)C2)[C@H]1C[C@@H](O)c2cc(Cl)ccc2O1] |
| 282 | 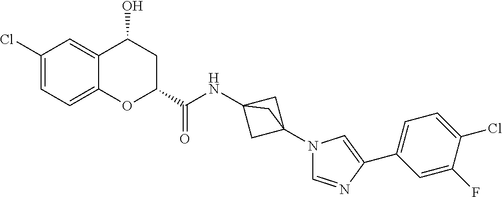 [SMILES: O=C(NC12CC(n3cnc(-c4ccc(Cl)c(F)c4)c3)(C1)C2)[C@H]1C[C@@H](O)c2cc(Cl)ccc2O1] |
| 283 | 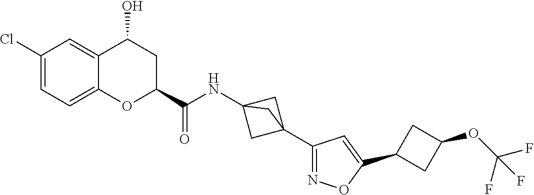 [SMILES: O=C(NC12CC(c3cc([C@H]4C[C@@H](OC(F)(F)F)C4)on3)(C1)C2)[C@@H]1C[C@@H](O)c2cc(Cl)ccc2O1] |
| 284 | 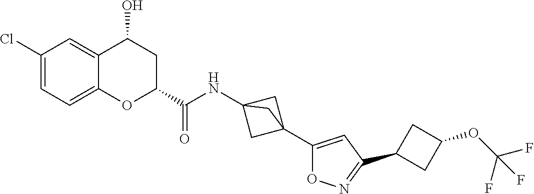 [SMILES: O=C(NC12CC(c3cc([C@H]4C[C@H](OC(F)(F)F)C4)no3)(C1)C2)[C@H]1C[C@@H](O)c2cc(Cl)ccc2O1] |
| 285 | 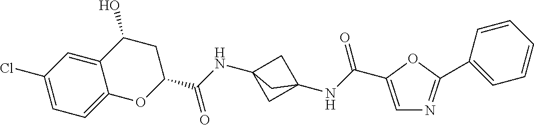 [SMILES: O=C(NC12CC(NC(=O)[C@H]3C[C@@H](O)c4cc(Cl)ccc4O3)(C1)C2)c1cnc(-c2ccccc2)o1] |
| 286 | 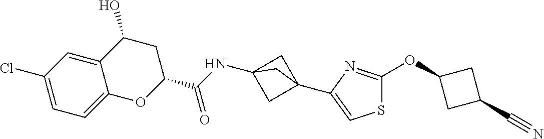 [SMILES: N#C[C@H]1C[C@@H](Oc2nc(C34CC(NC(=O)[C@H]5C[C@@H](O)c6cc(Cl)ccc6O5)(C3)C4)cs2)C1] |
| 287 | 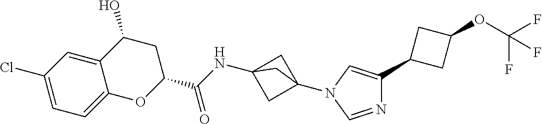 [SMILES: O=C(NC12CC(n3cnc([C@H]4C[C@@H](OC(F)(F)F)C4)c3)(C1)C2)[C@H]1C[C@@H](O)c2cc(Cl)ccc2O1] |
| 288 | 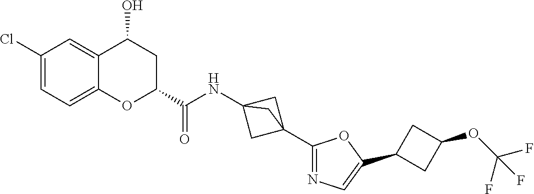 [SMILES: O=C(NC12CC(c3ncc([C@H]4C[C@@H](OC(F)(F)F)C4)o3)(C1)C2)[C@H]1C[C@@H](O)c2cc(Cl)ccc2O1] |
| 289 | 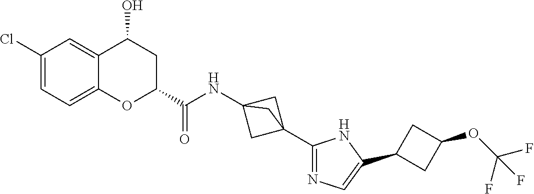 [SMILES: O=C(NC12CC(c3ncc([C@H]4C[C@@H](OC(F)(F)F)C4)[nH]3)(C1)C2)[C@H]1C[C@@H](O)c2cc(Cl)ccc2O1] |
| 290 | 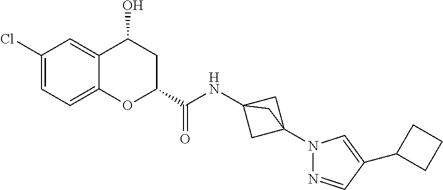 [SMILES: O=C(NC12CC(n3cc(C4CCC4)cn3)(C1)C2)[C@H]1C[C@@H](O)c2cc(Cl)ccc2O1] |
| 291 | 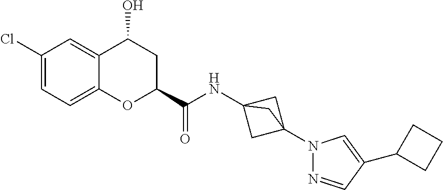 [SMILES: O=C(NC12CC(n3cc(C4CCC4)cn3)(C1)C2)[C@@H]1C[C@@H](O)c2cc(Cl)ccc2O1] |
| 292 | 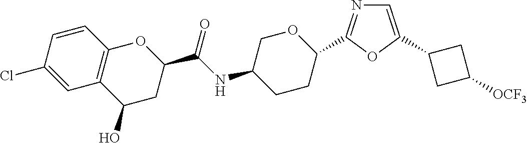 [SMILES: O=C(N[C@@H]1CC[C@@H](c2ncc([C@H]3C[C@@H](OC(F)(F)F)C3)o2)OC1)[C@H]1C[C@@H](O)c2cc(Cl)ccc2O1] |
| 293 | 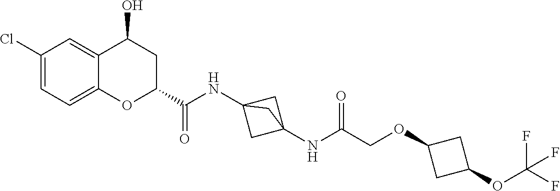 [SMILES: O=C(CO[C@H]1C[C@@H](OC(F)(F)F)C1)NC12CC(NC(=O)[C@H]3C[C@H](O)c4cc(Cl)ccc4O3)(C1)C2] |
| 294 | 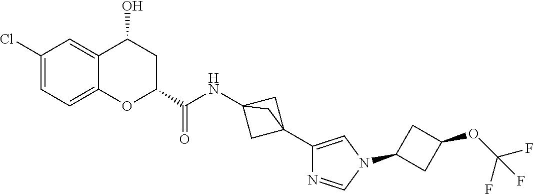 [SMILES: O=C(NC12CC(c3cn([C@H]4C[C@@H](OC(F)(F)F)C4)cn3)(C1)C2)[C@H]1C[C@@H](O)c2cc(Cl)ccc2O1] |
| 295 | 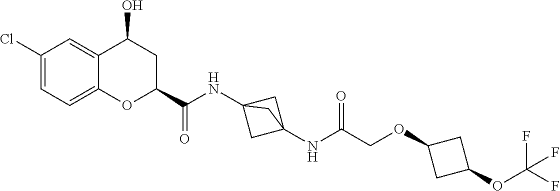 [SMILES: O=C(CO[C@H]1C[C@@H](OC(F)(F)F)C1)NC12CC(NC(=O)[C@@H]3C[C@H](O)c4cc(Cl)ccc4O3)(C1)C2] |
| 296 | 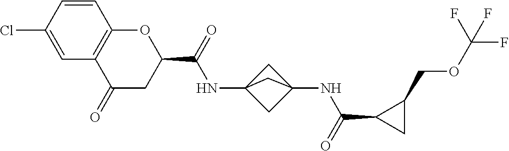 [SMILES: O=C1C[C@H](C(=O)NC23CC(NC(=O)[C@@H]4C[C@@H]4COC(F)(F)F)(C2)C3)Oc2ccc(Cl)cc21] |
| 297 | 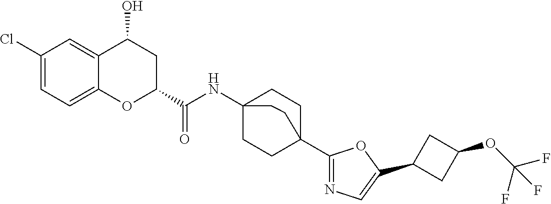 [SMILES: O=C(NC12CCC(c3ncc([C@H]4C[C@@H](OC(F)(F)F)C4)o3)(CC1)CC2)[C@H]1C[C@@H](O)c2cc(Cl)ccc2O1] |
| 298 | 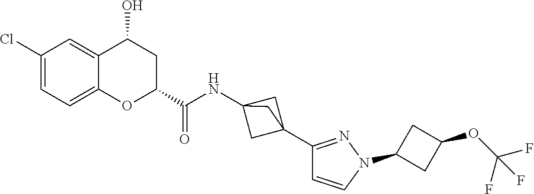 [SMILES: O=C(NC12CC(c3ccn([C@H]4C[C@@H](OC(F)(F)F)C4)n3)(C1)C2)[C@H]1C[C@@H](O)c2cc(Cl)ccc2O1] |
| 299 | 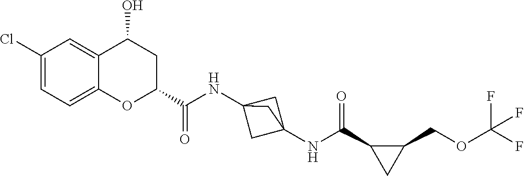 [SMILES: O=C(NC12CC(NC(=O)[C@@H]3C[C@@H]3COC(F)(F)F)(C1)C2)[C@H]1C[C@@H](O)c2cc(Cl)ccc2O1] |
| 300 | 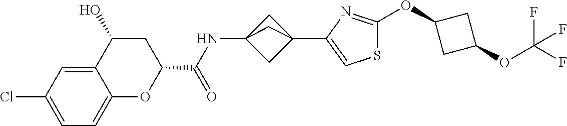 [SMILES: O=C(NC12CC(c3csc(O[C@H]4C[C@@H](OC(F)(F)F)C4)n3)(C1)C2)[C@H]1C[C@@H](O)c2cc(Cl)ccc2O1] |
| 301 | 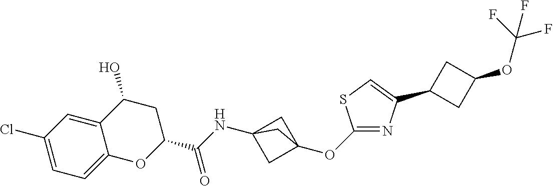 [SMILES: O=C(NC12CC(Oc3nc([C@H]4C[C@@H](OC(F)(F)F)C4)cs3)(C1)C2)[C@H]1C[C@@H](O)c2cc(Cl)ccc2O1] |
| 302 | 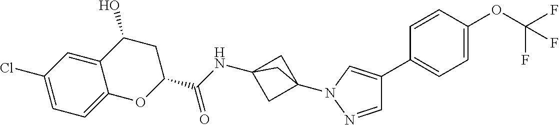 [SMILES: O=C(NC12CC(n3cc(-c4ccc(OC(F)(F)F)cc4)cn3)(C1)C2)[C@H]1C[C@@H](O)c2cc(Cl)ccc2O1] |
| 303 | 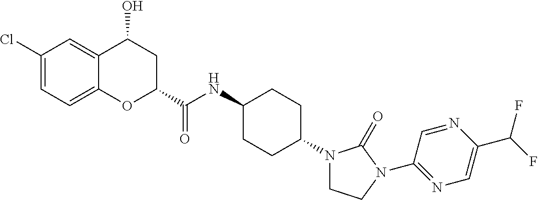 [SMILES: O=C(N[C@H]1CC[C@H](N2CCN(c3cnc(C(F)F)cn3)C2=O)CC1)[C@H]1C[C@@H](O)c2cc(Cl)ccc2O1] |
| 304 | 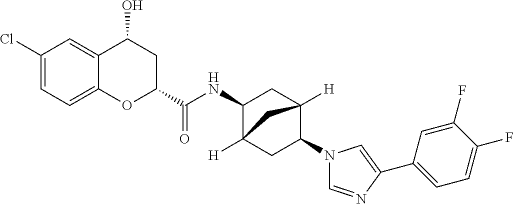 [SMILES: O=C(N[C@H]1C[C@H]2C[C@@H]1C[C@@H]2n1cnc(-c2ccc(F)c(F)c2)c1)[C@H]1C[C@@H](O)c2cc(Cl)ccc2O1] |
| 305 | 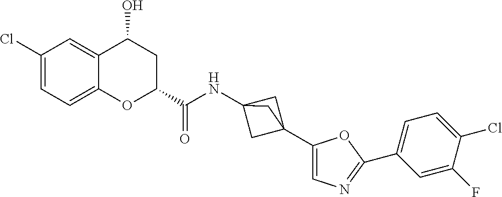 [SMILES: O=C(NC12CC(c3cnc(-c4ccc(Cl)c(F)c4)o3)(C1)C2)[C@H]1C[C@@H](O)c2cc(Cl)ccc2O1] |
| 306 | 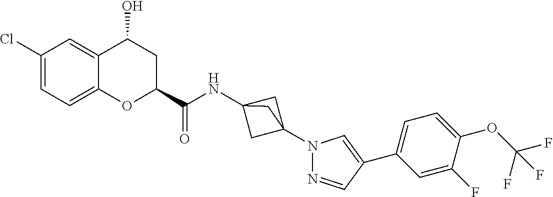 [SMILES: O=C(NC12CC(n3cc(-c4ccc(OC(F)(F)F)c(F)c4)cn3)(C1)C2)[C@@H]1C[C@@H](O)c2cc(Cl)ccc2O1] |
| 307 | 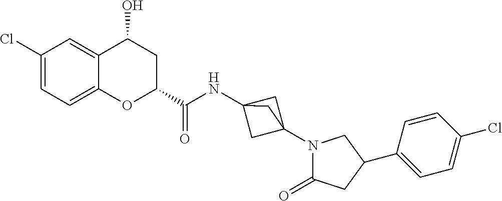 [SMILES: O=C(NC12CC(N3CC(c4ccc(Cl)cc4)CC3=O)(C1)C2)[C@H]1C[C@@H](O)c2cc(Cl)ccc2O1] |
| 308 | 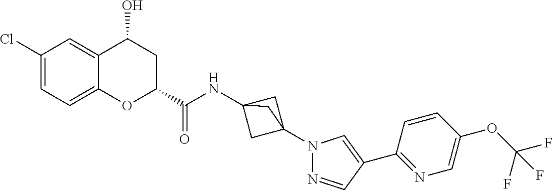 [SMILES: O=C(NC12CC(n3cc(-c4ccc(OC(F)(F)F)cn4)cn3)(C1)C2)[C@H]1C[C@@H](O)c2cc(Cl)ccc2O1] |
| 309 | 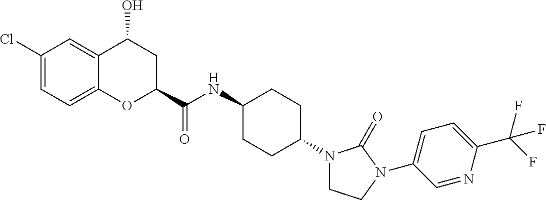 [SMILES: O=C(N[C@H]1CC[C@H](N2CCN(c3ccc(C(F)(F)F)nc3)C2=O)CC1)[C@@H]1C[C@@H](O)c2cc(Cl)ccc2O1] |
| 310 | 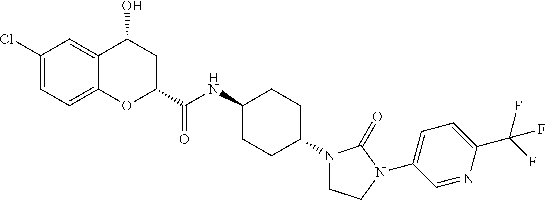 [SMILES: O=C(N[C@H]1CC[C@H](N2CCN(c3ccc(C(F)(F)F)nc3)C2=O)CC1)[C@H]1C[C@@H](O)c2cc(Cl)ccc2O1] |
| 311 | 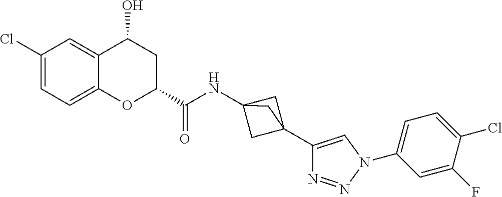 [SMILES: O=C(NC12CC(c3cn(-c4ccc(Cl)c(F)c4)nn3)(C1)C2)[C@H]1C[C@@H](O)c2cc(Cl)ccc2O1] |
| 312 | 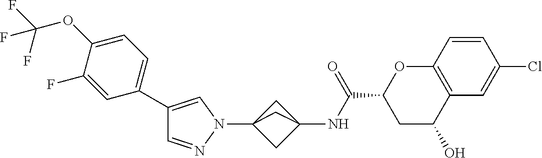 [SMILES: O=C(NC12CC(n3cc(-c4ccc(OC(F)(F)F)c(F)c4)cn3)(C1)C2)[C@H]1C[C@@H](O)c2cc(Cl)ccc2O1] |
| 313 | 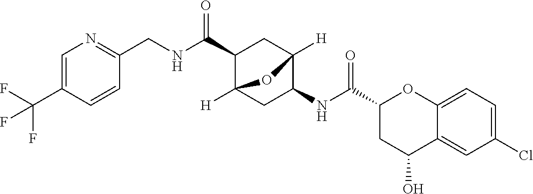 [SMILES: O=C(NCc1ccc(C(F)(F)F)cn1)[C@H]1C[C@H]2O[C@@H]1C[C@@H]2NC(=O)[C@H]1C[C@@H](O)c2cc(Cl)ccc2O1] |
| 314 | 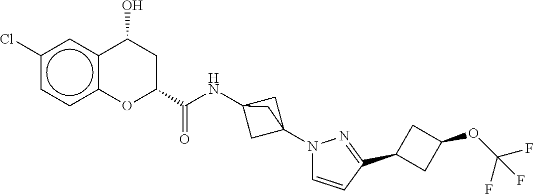 |
| 315 | 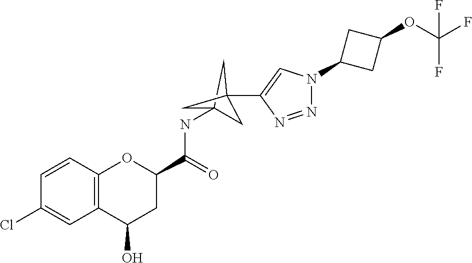 [SMILES: O=C(NC12CC(c3cn([C@H]4C[C@@H](OC(F)(F)F)C4)nn3)(C1)C2)[C@H]1C[C@@H](O)c2cc(Cl)ccc2O1] |
| 316 | 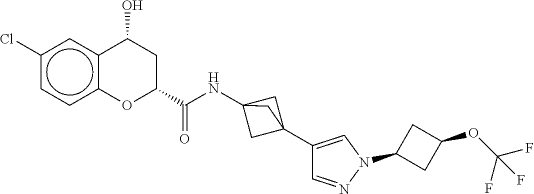 |

In some embodiments, a disclosed compound is selected from a compound set forth in Table 2 or a pharmaceutically acceptable salt, solvate, hydrate, tautomer, N-oxide or stereoisomer thereof.

TABLE 2
Exemplary compounds of the invention

| Compound | |
|---|---|
| Number | Structure |
| 317 | 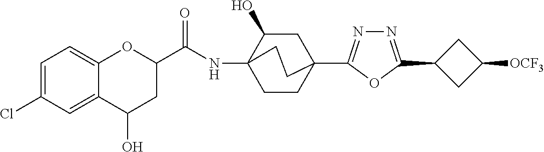 [SMILES: O=C(NC12CCC(c3nnc([C@H]4C[C@@H](OC(F)(F)F)C4)o3)(CC1)C[C@@H]2O)C1CC(O)c2cc(Cl)ccc2O1] |
| 318 | 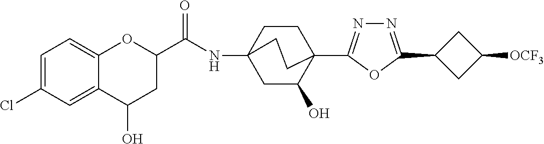 [SMILES: O=C(NC12CCC(c3nnc([C@H]4C[C@@H](OC(F)(F)F)C4)o3)(CC1)[C@@H](O)C2)C1CC(O)c2cc(Cl)ccc2O1] |
| 319 | 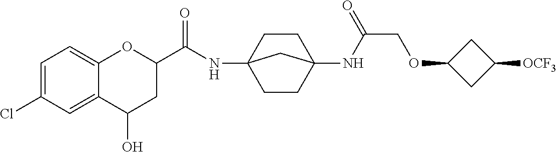 [SMILES: O=C(CO[C@H]1C[C@@H](OC(F)(F)F)C1)NC12CCC(NC(=O)C3CC(O)c4cc(Cl)ccc4O3)(CC1)C2] |
| 320 | 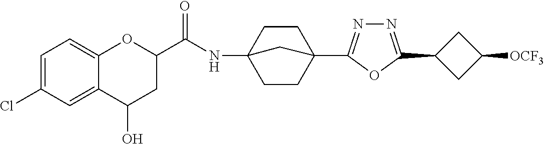 [SMILES: O=C(NC12CCC(c3nnc([C@H]4C[C@@H](OC(F)(F)F)C4)o3)(CC1)C2)C1CC(O)c2cc(Cl)ccc2O1] |
| 321 | 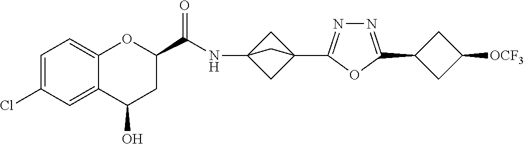 [SMILES: O=C(NC12CC(c3nnc([C@H]4C[C@@H](OC(F)(F)F)C4)o3)(C1)C2)[C@H]1C[C@@H](O)c2cc(Cl)ccc2O1] |
| 322 | 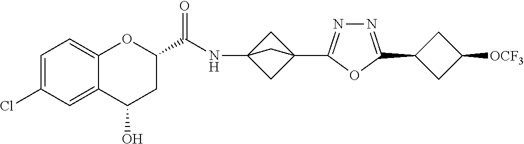 [SMILES: O=C(NC12CC(c3nnc([C@H]4C[C@@H](OC(F)(F)F)C4)o3)(C1)C2)[C@@H]1C[C@H](O)c2cc(Cl)ccc2O1] |
| 323 | 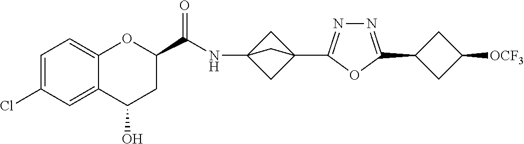 [SMILES: O=C(NC12CC(c3nnc([C@H]4C[C@@H](OC(F)(F)F)C4)o3)(C1)C2)[C@H]1C[C@H](O)c2cc(Cl)ccc2O1] |
| 324 | 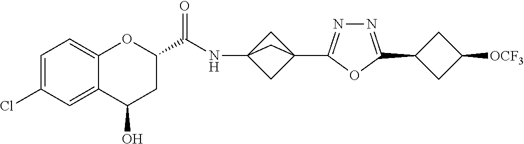 [SMILES: O=C(NC12CC(c3nnc([C@H]4C[C@@H](OC(F)(F)F)C4)o3)(C1)C2)[C@@H]1C[C@@H](O)c2cc(Cl)ccc2O1] |
| 325 | 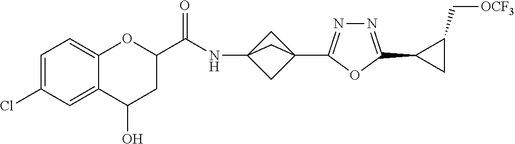 [SMILES: O=C(NC12CC(c3nnc([C@@H]4C[C@H]4COC(F)(F)F)o3)(C1)C2)C1CC(O)c2cc(Cl)ccc2O1] |
| 326 | 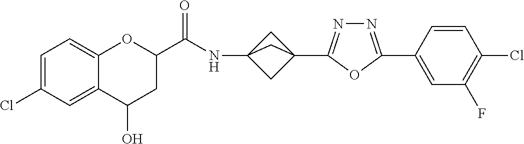 [SMILES: O=C(NC12CC(c3nnc(-c4ccc(Cl)c(F)c4)o3)(C1)C2)C1CC(O)c2cc(Cl)ccc2O1] |
| 327 | 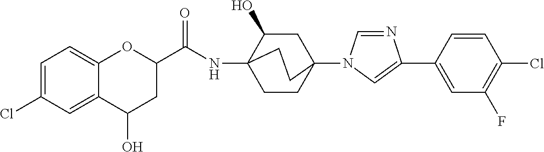 [SMILES: O=C(NC12CCC(n3cnc(-c4ccc(Cl)c(F)c4)c3)(CC1)C[C@@H]2O)C1CC(O)c2cc(Cl)ccc2O1] |
| 328 | 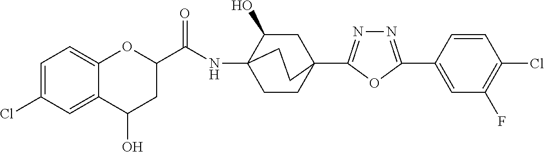 [SMILES: O=C(NC12CCC(c3nnc(-c4ccc(Cl)c(F)c4)o3)(CC1)C[C@@H]2O)C1CC(O)c2cc(Cl)ccc2O1] |
| 329 | 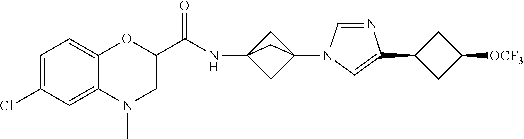 [SMILES: CN1CC(C(=O)NC23CC(n4cnc([C@H]5C[C@@H](OC(F)(F)F)C5)c4)(C2)C3)Oc2ccc(Cl)cc21] |
| 330 | 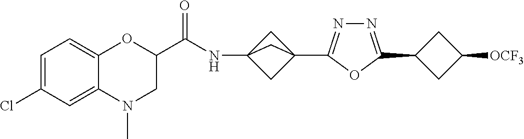 [SMILES: CN1CC(C(=O)NC23CC(c4nnc([C@H]5C[C@@H](OC(F)(F)F)C5)o4)(C2)C3)Oc2ccc(Cl)cc21] |
| 331 | 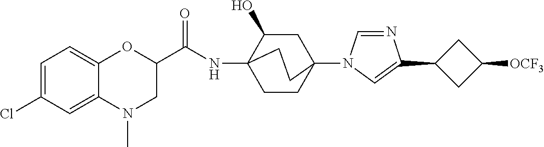 [SMILES: CN1CC(C(=O)NC23CCC(n4cnc([C@H]5C[C@@H](OC(F)(F)F)C5)c4)(CC2)C[C@@H]3O)Oc2ccc(Cl)cc21] |
| 332 | 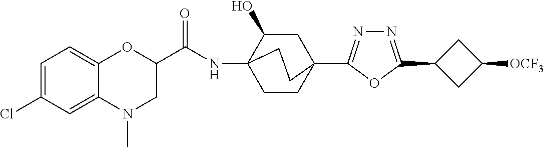 [SMILES: CN1CC(C(=O)NC23CCC(c4nnc([C@H]5C[C@@H](OC(F)(F)F)C5)o4)(CC2)C[C@@H]3O)Oc2ccc(Cl)cc21] |
| 333 | 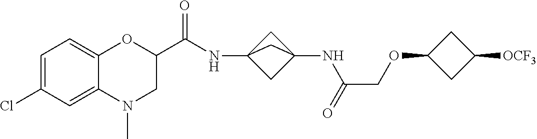 [SMILES: CN1CC(C(=O)NC23CC(NC(=O)CO[C@H]4C[C@@H](OC(F)(F)F)C4)(C2)C3)Oc2ccc(Cl)cc21] |
| 334 | 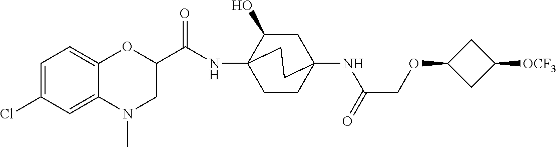 [SMILES: CN1CC(C(=O)NC23CCC(NC(=O)CO[C@H]4C[C@@H](OC(F)(F)F)C4)(CC2)C[C@@H]3O)Oc2ccc(Cl)cc21] |
| 335 | 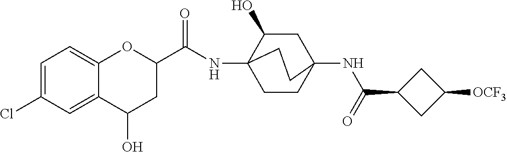 [SMILES: O=C(NC12CCC(NC(=O)[C@H]3C[C@@H](OC(F)(F)F)C3)(CC1)C[C@@H]2O)C1CC(O)c2cc(Cl)ccc2O1] |
| 336 | 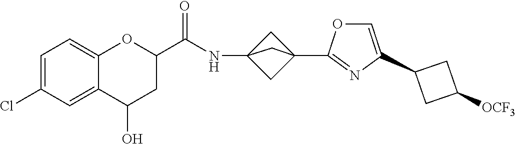 [SMILES: O=C(NC12CC(c3nc([C@H]4C[C@@H](OC(F)(F)F)C4)co3)(C1)C2)C1CC(O)c2cc(Cl)ccc2O1] |
| 337 | 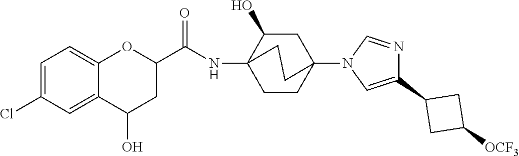 [SMILES: O=C(NC12CCC(n3cnc([C@H]4C[C@@H](OC(F)(F)F)C4)c3)(CC1)C[C@@H]2O)C1CC(O)c2cc(Cl)ccc2O1] |
| 338 | 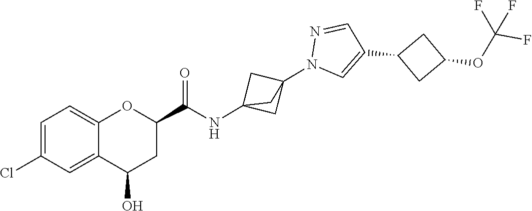 [SMILES: O=C(NC12CC(n3cc([C@H]4C[C@@H](OC(F)(F)F)C4)cn3)(C1)C2)[C@H]1C[C@@H](O)c2cc(Cl)ccc2O1] |
| 339 | 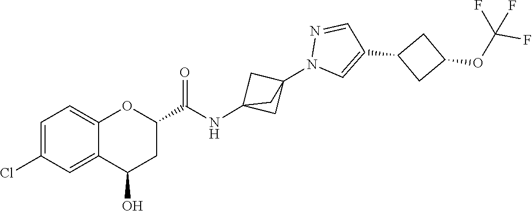 [SMILES: O=C(NC12CC(n3cc([C@H]4C[C@@H](OC(F)(F)F)C4)cn3)(C1)C2)[C@@H]1C[C@@H](O)c2cc(Cl)ccc2O1] |
| 340 | 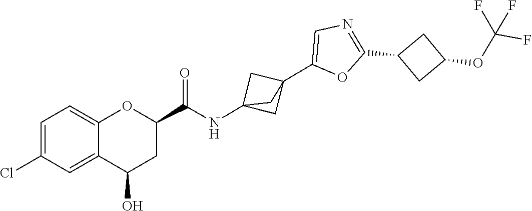 [SMILES: O=C(NC12CC(c3cnc([C@H]4C[C@@H](OC(F)(F)F)C4)o3)(C1)C2)[C@H]1C[C@@H](O)c2cc(Cl)ccc2O1] |
| 341 | 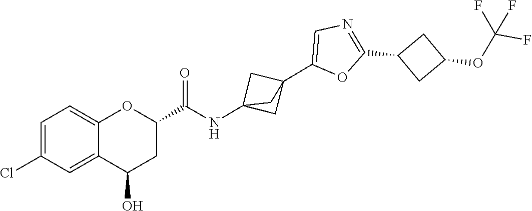 [SMILES: O=C(NC12CC(c3cnc([C@H]4C[C@@H](OC(F)(F)F)C4)o3)(C1)C2)[C@@H]1C[C@@H](O)c2cc(Cl)ccc2O1] |
| 342 | 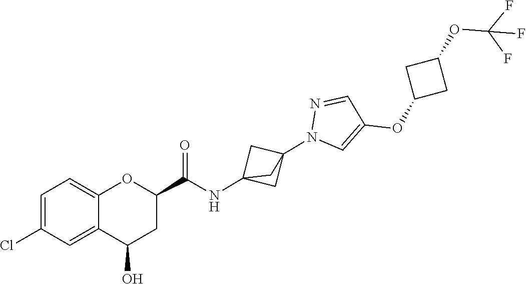 [SMILES: O=C(NC12CC(n3cc(O[C@H]4C[C@@H](OC(F)(F)F)C4)cn3)(C1)C2)[C@H]1C[C@@H](O)c2cc(Cl)ccc2O1] |
| 343 | 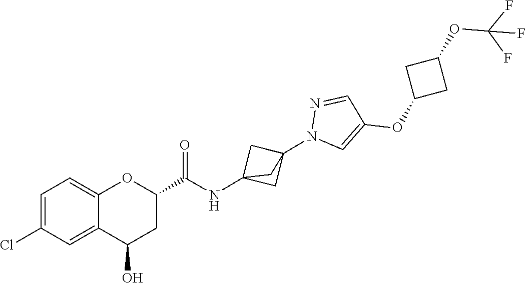 [SMILES: O=C(NC12CC(n3cc(O[C@H]4C[C@@H](OC(F)(F)F)C4)cn3)(C1)C2)[C@@H]1C[C@@H](O)c2cc(Cl)ccc2O1] |
| 344 | 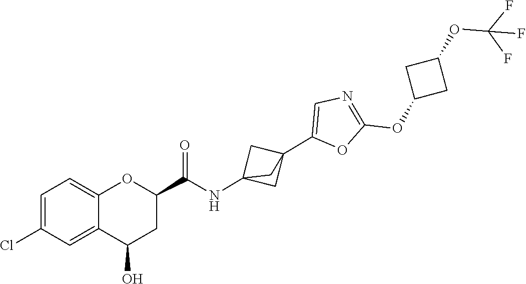 [SMILES: O=C(NC12CC(c3cnc(O[C@H]4C[C@@H](OC(F)(F)F)C4)o3)(C1)C2)[C@H]1C[C@@H](O)c2cc(Cl)ccc2O1] |
| 345 | 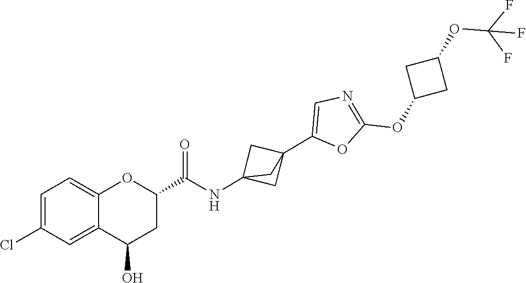 [SMILES: O=C(NC12CC(c3cnc(O[C@H]4C[C@@H](OC(F)(F)F)C4)o3)(C1)C2)[C@@H]1C[C@@H](O)c2cc(Cl)ccc2O1] |
| 346 | 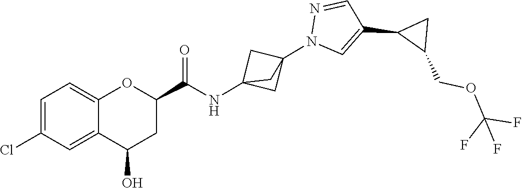 [SMILES: O=C(NC12CC(n3cc([C@H]4C[C@@H]4COC(F)(F)F)cn3)(C1)C2)[C@H]1C[C@@H](O)c2cc(Cl)ccc2O1] |
| 347 | 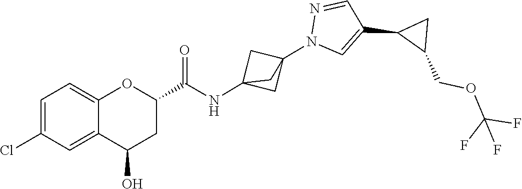 [SMILES: O=C(NC12CC(n3cc([C@H]4C[C@@H]4COC(F)(F)F)cn3)(C1)C2)[C@@H]1C[C@@H](O)c2cc(Cl)ccc2O1] |
| 348 | 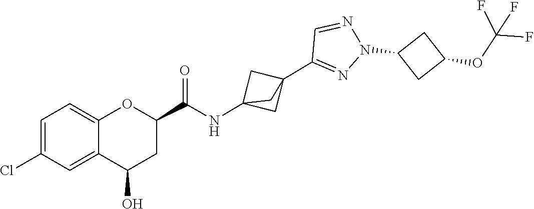 [SMILES: O=C(NC12CC(c3cnn([C@H]4C[C@@H](OC(F)(F)F)C4)n3)(C1)C2)[C@H]1C[C@@H](O)c2cc(Cl)ccc2O1] |
| 349 | 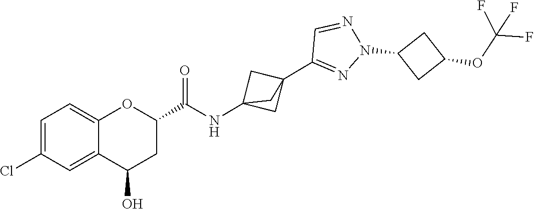 [SMILES: O=C(NC12CC(c3cnn([C@H]4C[C@@H](OC(F)(F)F)C4)n3)(C1)C2)[C@@H]1C[C@@H](O)c2cc(Cl)ccc2O1] |
| 350 | 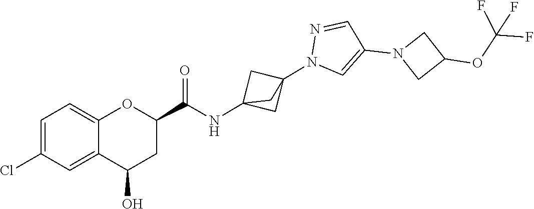 [SMILES: O=C(NC12CC(n3cc(N4CC(OC(F)(F)F)C4)cn3)(C1)C2)[C@H]1C[C@@H](O)c2cc(Cl)ccc2O1] |
| 351 | 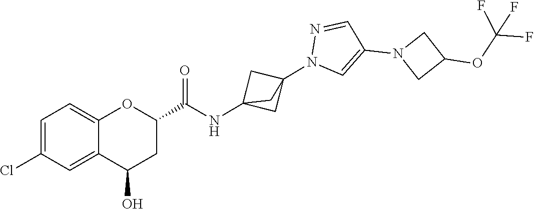 [SMILES: O=C(NC12CC(n3cc(N4CC(OC(F)(F)F)C4)cn3)(C1)C2)[C@@H]1C[C@@H](O)c2cc(Cl)ccc2O1] |
| 352 | 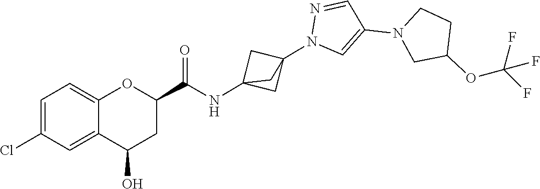 [SMILES: O=C(NC12CC(n3cc(N4CCC(OC(F)(F)F)C4)cn3)(C1)C2)[C@H]1C[C@@H](O)c2cc(Cl)ccc2O1] |
| 353 | 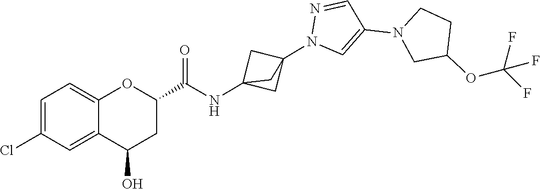 [SMILES: O=C(NC12CC(n3cc(N4CCC(OC(F)(F)F)C4)cn3)(C1)C2)[C@@H]1C[C@@H](O)c2cc(Cl)ccc2O1] |
| 354 | 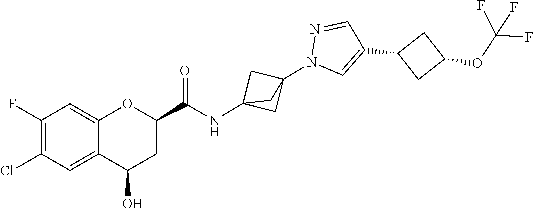 [SMILES: O=C(NC12CC(n3cc([C@H]4C[C@@H](OC(F)(F)F)C4)cn3)(C1)C2)[C@H]1C[C@@H](O)c2cc(Cl)c(F)cc2O1] |
| 355 | 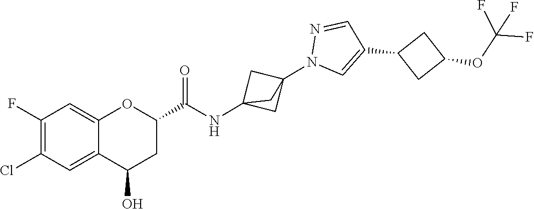 [SMILES: O=C(NC12CC(n3cc([C@H]4C[C@@H](OC(F)(F)F)C4)cn3)(C1)C2)[C@@H]1C[C@@H](O)c2cc(Cl)c(F)cc2O1] |
| 356 | 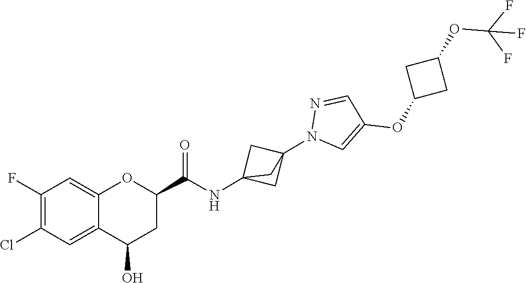 [SMILES: O=C(NC12CC(n3cc(O[C@H]4C[C@@H](OC(F)(F)F)C4)cn3)(C1)C2)[C@H]1C[C@@H](O)c2cc(Cl)c(F)cc2O1] |
| 357 | 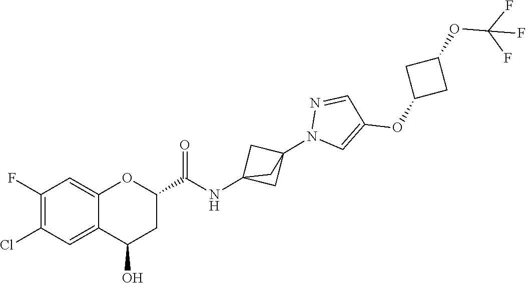 [SMILES: O=C(NC12CC(n3cc(O[C@H]4C[C@@H](OC(F)(F)F)C4)cn3)(C1)C2)[C@@H]1C[C@@H](O)c2cc(Cl)c(F)cc2O1] |

### Methods of Making Exemplary Compounds

The compounds of the invention may be better understood in connection with the following synthetic schemes and methods which illustrate a means by which the compounds can be prepared. The compounds of this invention can be prepared by a variety of synthetic procedures. A representative synthetic procedure is illustrated in, but is not limited to, that shown in the following schemes. The variables A, D, E, W, X, Y, L, L¹, L², R¹, R², R^{W2}, A^{II}, D^{II}, W^{II}, Y^{II}, L^{1-II}, L^{2-II}, R^{1-II}, R^{2-II}, A^{III}, D^{III}, W^{III}, L^{1-III}, L^{2-III}, R^{1-III}, and R^{2-III} are defined as detailed herein, e.g., in the Summary.

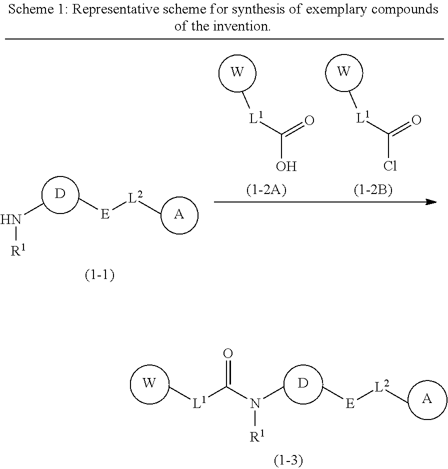

As shown in Scheme 1, compounds of formula (1-3) can be prepared from compounds of formula (1-1). Compounds of formula (1-1) can be coupled with carboxylic acids of formula (1-2A) or alternatively with acid chlorides of formula (1-2B) under amide bond forming conditions to give amides of formula (1-3). Examples of conditions known to generate amides from a mixture of a carboxylic acid of formula (1-2A) and an amine of formula (1-1) include but are not limited to adding a coupling reagent such as N-(3-dimethylaminopropyl)-N′-ethylcarbodiimide or 1-(3-dimethylaminopropyl)-3-ethylcarbodiimide (EDC, EDAC or EDCI), 1,3-dicyclohexylcarbodiimide (DCC), bis(2-oxo-3-oxazolidinyl)phosphinic chloride (BOPC), N-[(dimethylamino)-1H-1,2,3-triazolo-[4,5-b]pyridin-1-ylmethylene]-N-methylmethanaminium hexafluorophosphate N-oxide or 2-(7-azabenzotriazol-1-yl)-N,N,N′,N′-tetramethyluronium hexafluorophosphate or 1-[bis(dimethylamino)methylene]-1H-1,2,3-triazolo[4,5-b]pyridinium 3-oxid hexafluorophosphate or 2-(3H-[1,2,3]triazolo[4,5-b]pyridin-3-yl)-1,1,3,3-tetramethylisouronium hexafluorophosphate(V) or 2-(7-aza-1H-benzotriazole-1-yl)-1,1,3,3-tetramethyluronium hexafluorophosphate (HATU), O-(benzotriazol-1-yl)-N,N,N′,N′-tetramethyluronium tetrafluoroborate (TBTU), 2-(1H-benzo[d][1,2,3]triazol-1-yl)-1,1,3,3-tetramethylisouronium hexafluorophosphate(V) (HBTU), 2,4,6-tripropyl-1,3,5,2,4,6-trioxatriphosphinane 2,4,6-trioxide (T3P®), (1-cyano-2-ethoxy-2-oxoethylidenaminooxy)dimethylamino-morpholino-carbenium hexafluorophosphate (COMU®), and fluoro-N,N,N′,N′-tetramethylformamidinium hexafluorophosphate. The coupling reagents may be added as a solid, a solution, or as the reagent bound to a solid support resin.

In addition to the coupling reagents, auxiliary-coupling reagents may facilitate the coupling reaction. Auxiliary coupling reagents that are often used in the coupling reactions include but are not limited to 4-(dimethylamino)pyridine (DMAP), 1-hydroxy-7-azabenzotriazole (HOAT) and 1-hydroxybenzotriazole (HOBT). The coupling reaction may be carried out optionally in the presence of a base such as triethylamine or diisopropylethylamine. The coupling reaction may be carried out in solvents such as but not limited to tetrahydrofuran, N,N-dimethylformamide, N,N-dimethylacetamide, dimethyl sulfoxide, dichloromethane, and ethyl acetate.

Alternatively, carboxylic acids of formula (1-2A) can be converted to the corresponding acid chlorides of formula (1-2B) by reaction with thionyl chloride, PCl₃, PCl₅, cyanuric chloride, Ghosez's reagent or oxalyl chloride. The reactions with thionyl chloride and oxalyl chloride can be catalyzed with N,N-dimethylformamide at ambient temperature in a solvent such as dichloromethane. The resultant acid chlorides of formula (1-2B) can then be coupled with amines of formula (1-1) optionally in the presence of a base such as a tertiary amine base such as triethylamine or diisopropylethylamine or an aromatic base such as pyridine, at room temperature in a solvent such as dichloromethane to give amides of formula (1-3). Compounds of formula (1-3) are representative of compounds of formula (I).

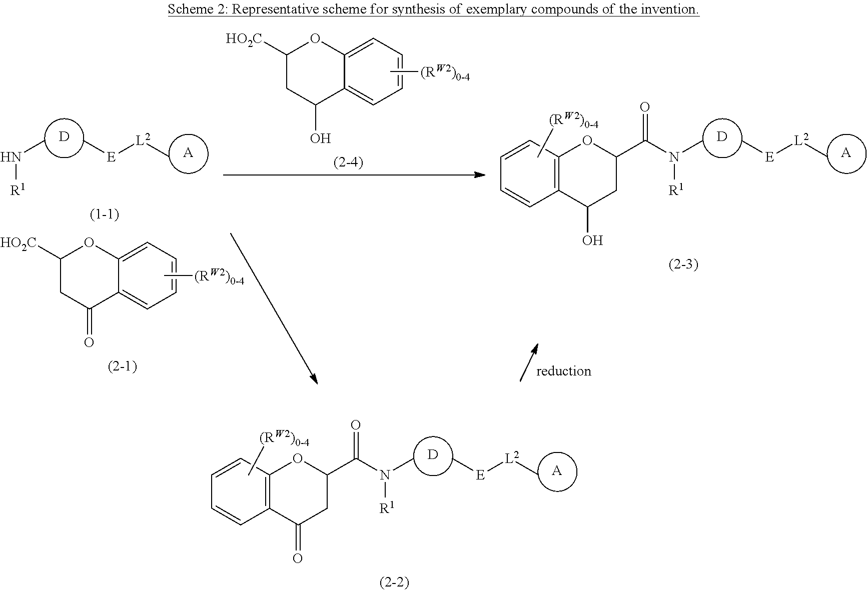

As shown in Scheme 2, compounds of formula (2-3) can be prepared from compounds of formula (1-1). Compounds of formula (1-1) can be coupled with compounds of formula (2-1), under amide bond forming conditions described in Scheme 1 to give compounds of formula (2-2). Compounds of formula (2-2) can be reduced to compounds of formula (2-3) using a reductant such as sodium cyanoborohydride in the presence of zinc chloride in an optionally warmed solvent such as methanol or sodium borohydride in a solvent such as methanol. Compounds of formula (2-2) and formula (2-3) are representative of compounds of Formula (I).

Alternatively, compounds of formula (1-1) can be coupled with compounds of formula (2-4), under amide bond forming conditions described in Scheme 1 to give compounds of formula (2-3).

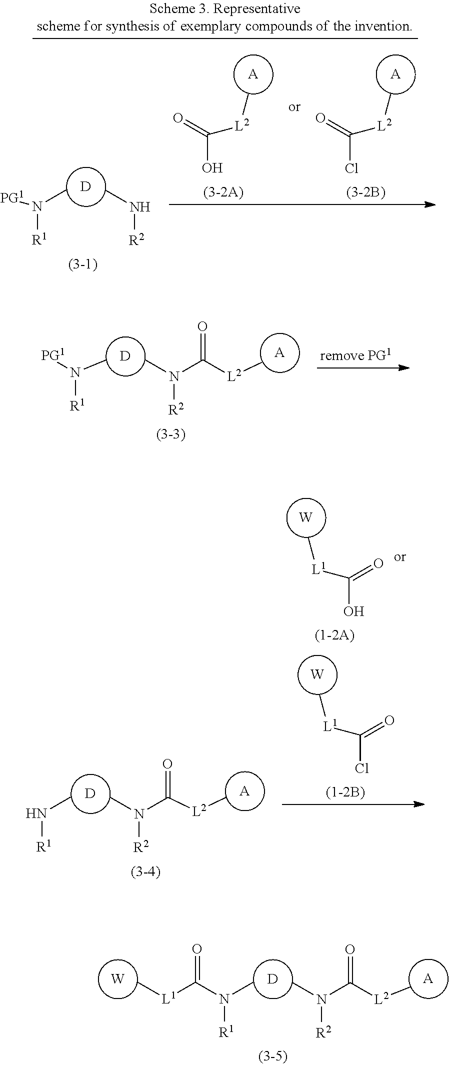

As shown in Scheme 3, compounds of formula (3-5) can be prepared from compounds of formula (3-1). Compounds of formula (3-1) where PG¹ is an amine protecting group (e.g. tert-butoxycarbonyl or benzyloxycarbonyl) can be coupled with carboxylic acids of formula (3-2A) or alternatively with acid chlorides of formula (3-2B) under amide bond forming conditions to give amides of formula (3-3). Examples of conditions known to generate amides from a mixture of a carboxylic acid of formula (3-2A) and an amine of formula (3-1) are described in Scheme 1.

Alternatively, carboxylic acids of formula (3-2A) can be converted to the corresponding acid chlorides of formula (3-2B) by reactions described in Scheme 1. The resultant acid chlorides of formula (3-2B) can then be coupled with amines of formula (3-1) optionally in the presence of a base such as a tertiary amine base such as triethylamine or diisopropylethylamine or an aromatic base such as pyridine, at room temperature in a solvent such as dichloromethane to give amides of formula (3-3).

Compounds of formula (3-3) can be deprotected using conditions known to one of skill in the art and dependent upon the protecting group (PG¹) used to give compounds of formula (3-4). Compounds of formula (3-4) can be coupled with carboxylic acids of formula (1-2A) or alternatively acid chlorides of formula (1-2B) under amide bond forming conditions as discussed above to afford compounds of formula (3-5). Compounds of formula (3-5) are representative compounds of Formula (I).

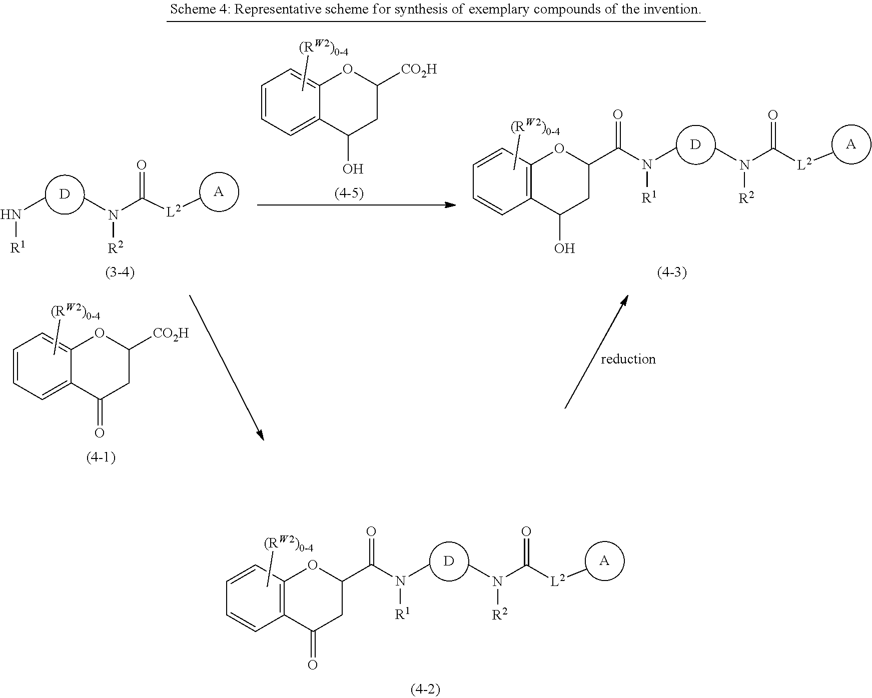

As shown in Scheme 4, compounds of formula (4-3) can be prepared from compounds of formula (3-4). Compounds of formula (3-4) can be coupled with compounds of formula (4-1), under amide bond forming conditions described in Scheme 1 to give compounds of formula (4-2). Compounds of formula (4-2) can be reduced to compounds of formula (4-3) using conditions described in Scheme 2. Compounds of formula (4-2) and formula (4-3) are representative of compounds of Formula (I).

Alternatively, compounds of formula (3-4) can be coupled with compounds of formula (4-5), under amide bond forming conditions described in Scheme 1 to give compounds of formula (4-3).

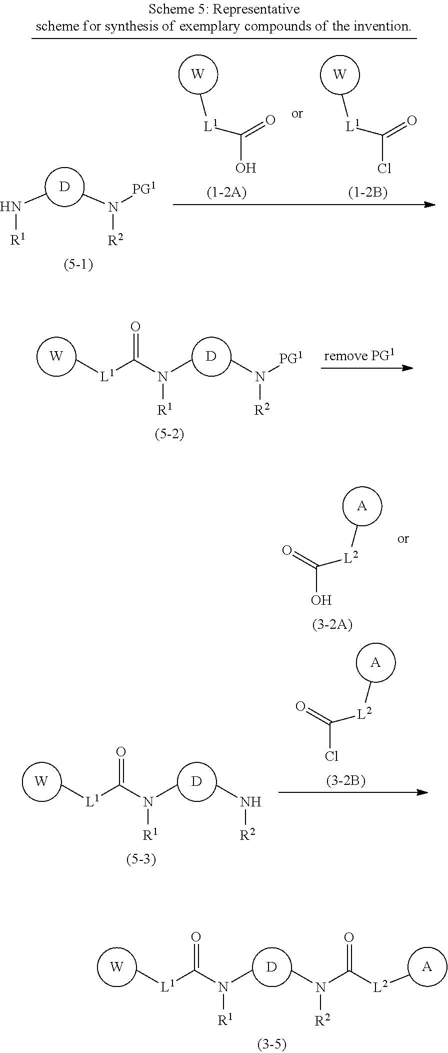

As shown in Scheme 5, compounds of formula (3-5) can be prepared from compounds of formula (5-1). Compounds of formula (5-1) where PG is an amine protecting group (e.g. tert-butoxycarbonyl or benzyloxycarbonyl) can be coupled with carboxylic acids of formula (1-2A) or alternatively with acid chlorides of formula (1-2B) under amide bond forming conditions to give amides of formula (5-2). Examples of conditions known to generate amides from a mixture of a carboxylic acid of formula (1-2A) and an amine of formula (5-1) are described in Scheme 1.

Alternatively, carboxylic acids of formula (1-2A) can be converted to the corresponding acid chlorides of formula (1-2B) by reactions described in Scheme 1. The resultant acid chlorides of formula (1-2B) can then be coupled with amines of formula (5-1) optionally in the presence of a base such as a tertiary amine base such as triethylamine or diisopropylethylamine or an aromatic base such as pyridine, at room temperature in a solvent such as dichloromethane to give amides of formula (5-2).

Compounds of formula (5-2) can be deprotected using conditions known to one of skill in the art and dependent upon the protecting group (PG¹) used to give compounds of formula (5-3). Compounds of formula (5-3) can be coupled with carboxylic acids of formula (3-2A) or alternatively acid chlorides of formula (3-2B) under amide bond forming conditions as discussed above to afford compounds of formula (3-5). Compounds of formula (3-5) are representative compounds of Formula (I).

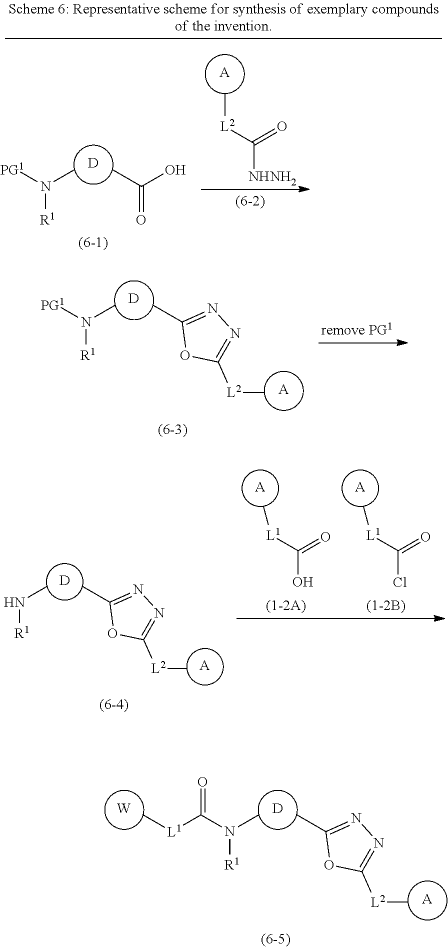

Compounds of formula (6-1) can be reacted with compounds of formula (6-2) in heated phosphorus oxychloride to give compounds of formula (6-3). Alternatively, compounds of formula (6-1) can also be reacted with compounds of formula (6-2) under the amide bond coupling conditions described to make compounds of formula (1-3). Following the coupling, the intermediate can be cyclized and dehydrated using 4-methylbenzene-1-sulfonyl chloride in the presence of a tertiary amine base such as N,N-diisopropylethylamine in optionally heated acetonitrile to give compounds of formula (6-3). Compounds of formula (6-3) can be deprotected using conditions known to one of skill in the art and dependent upon the protecting group (PG¹) used to give compounds of formula (6-4). Compounds of formula (6-4) can be coupled with carboxylic acids of formula (1-2A) or alternatively acid chlorides of formula (1-2B) under amide bond forming conditions as discussed above to afford compounds of formula (6-5). Compounds of formula (6-5) are representative compounds of Formula (I).

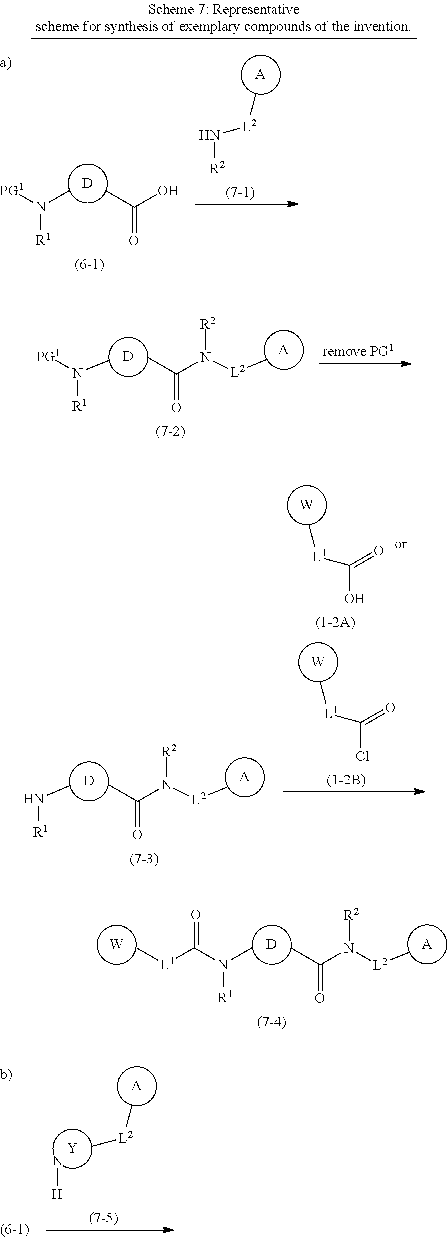

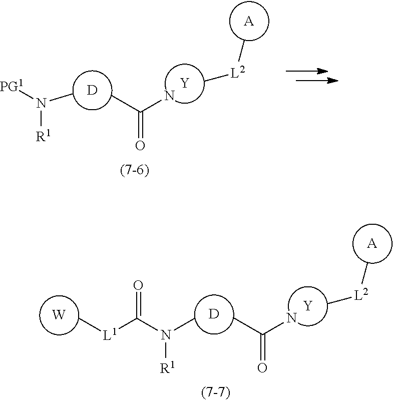

As shown in Scheme 7 a), compounds of formula (7-4) can be prepared from compounds of formula (6-1). Compounds of formula (6-1) where PG¹ is an amine protecting group (e.g. tert-butoxycarbonyl or benzyloxycarbonyl) can be coupled with amines of formula (7-1) under amide bond forming conditions to give amides of formula (7-2). Examples of conditions known to generate amides from a mixture of a carboxylic acid of formula (6-1) and an amine of formula (7-1) are described in Scheme 1.

Compounds of formula (7-2) can be deprotected using conditions known to one of skill in the art and dependent upon the protecting group (PG¹) used to give compounds of formula (7-3). Compounds of formula (7-3) can be coupled with carboxylic acids of formula (1-2A) or alternatively acid chlorides of formula (1-2B) under amide bond forming conditions as discussed above to afford compounds of formula (7-4). Compounds of formula (7-4) are representative compounds of Formula (I). As shown in Scheme 7 b), compounds of formula (7-7) can be prepared from compounds of formula (6-1) and amines of formula (7-5) using the reaction conditions described in Scheme 7a. Compounds of formula (7-7) are representative compounds of Formula (I).

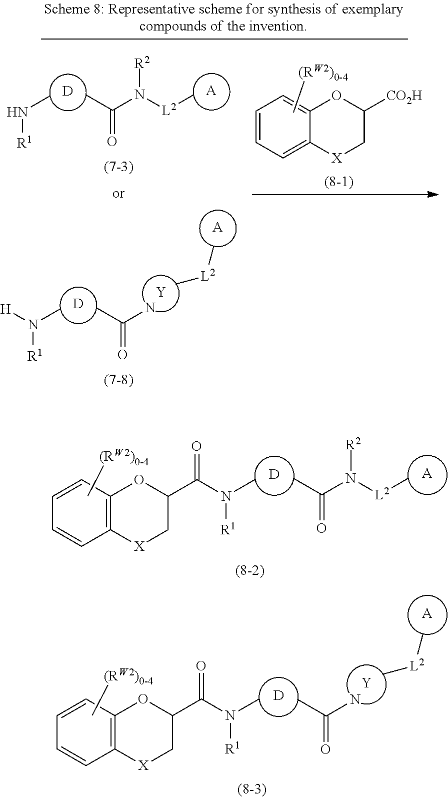

As shown in Scheme 8, compounds of formula (8-2) or formula (8-3) can be prepared from compounds of formula (7-3) and formula (7-8) respectively. Compounds of formula (7-3) or formula (7-8) can be coupled with compounds of formula (8-1), under amide bond forming conditions described in Scheme 1 to give compounds of formula (8-2) or compounds of formula (8-3). Compounds of formula (8-2) and formula (8-3) are representative of compounds of formula (I)

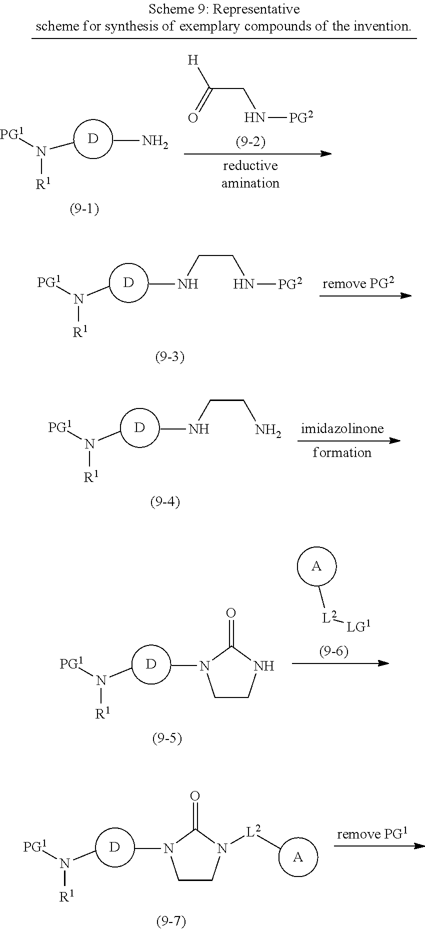

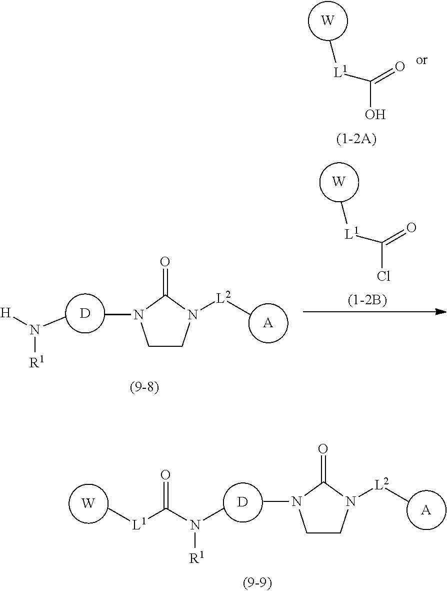

As shown in Scheme 9, compounds of formula (9-9) can be prepared from compounds of formula (9-1). Compounds of formula (9-1) can be reductively aminated with compounds of formula (9-2), wherein PG¹ is a suitable amine protecting group, to afford compounds of formula (9-3). Removal of the amine protecting group of compounds of formula (9-3) using conditions known to one of skill in the art and dependent upon the protecting group (PG¹) affords compounds of formula (9-4) which can subsequently be cyclized via imidazolinone forming conditions utilizing the primary and secondary amine groups to afford compounds of formula (9-5). Compounds of formula (9-4) can be treated with a carbonylation reagent such as A N,N′-carbonyldiimidazole in the presence of a tertiary amine base such as 1,8-diazabicyclo[5.4.0]undec-7-ene. Compounds of formula (9-5) can be treated with compounds of formula (9-6) where LG¹ is a leaving group, e.g., halogen or sulfonate, under nucleophilic substitution (when L² is a bond) to give compounds of formula (9-7). When L² is a bond, nuclear aromatic substitution reaction conditions may be used such as palladium catalyzed cross-coupling reaction conditions of compounds of formula (9-5) with compounds of formula (9-6) to give compounds of formula (9-7). An example of palladium cross-coupling reaction conditions includes but is not limited to a palladium catalyst (e.g. tris(dibenzylideneacetone)dipalladium(0)), a ligand (e.g. 2-(dicyclohexylphosphino)-2′,4′,6′-triisopropylbiphenyl (XPhos)), and a base (e.g. cesium carbonate), heated in a solvent (e.g. dioxane) under an inert atmosphere. Compounds of formula (9-9) are representative compounds of Formula (I).

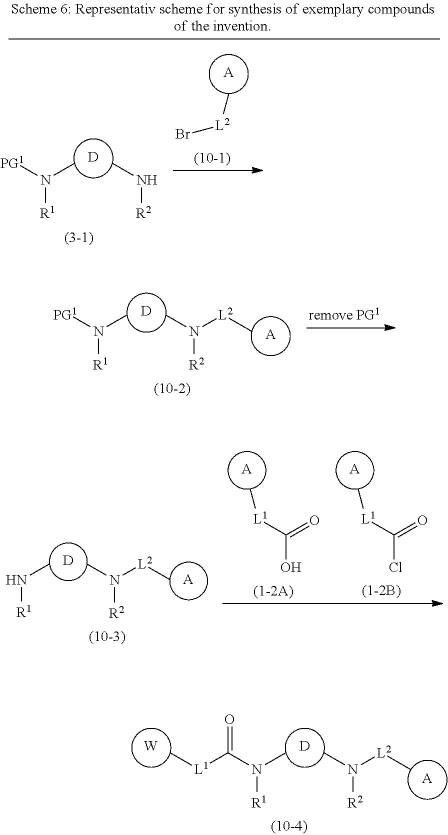

Alternatively, compounds of formula (10-4), can be prepared from compounds of formula (3-1) as shown in Scheme 10. Amines of formula (3-1) can be reacted with bromides of formula (10-1), in the presence of a base such as, but not limited to, N,N-diisopropylethylamine, or potassium carbonate, to provide compounds of formula (10-2). The reaction is typically performed at an elevated temperature in a solvent such as, but not limited to, N,N-dimethylformamide or dimethyl sulfoxide.

Compounds of formula (10-2) can be deprotected using conditions known to one of skill in the art and dependent upon the protecting group (PG¹) used to give compounds of formula (10-3). Compounds of formula (10-3) can be coupled with carboxylic acids of formula (1-2A) or alternatively acid chlorides of formula (1-2B) under amide bond forming conditions as discussed above to afford compounds of formula (10-4). Compounds of formula (10-4) are representative compounds of Formula (I).

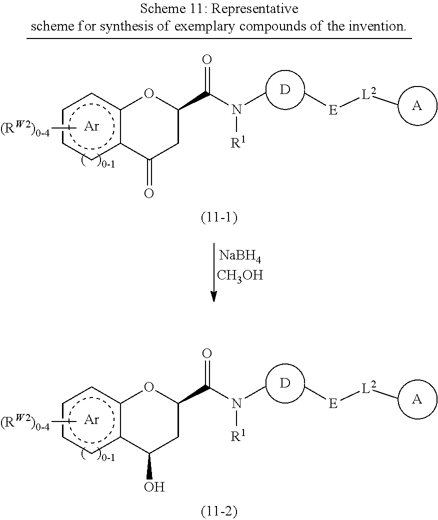

As shown in Scheme 11, compounds of formula (11-2) can be prepared from compounds of formula (11-1). Compounds of formula (11-1), wherein Ar is a fused aryl or heteroaryl ring, can be reduced to compounds of formula (11-2) using a reductant such as sodium borohydride in an optionally warmed solvent such as methanol. Compounds of formula (11-2) are representative of compounds of Formula (I).

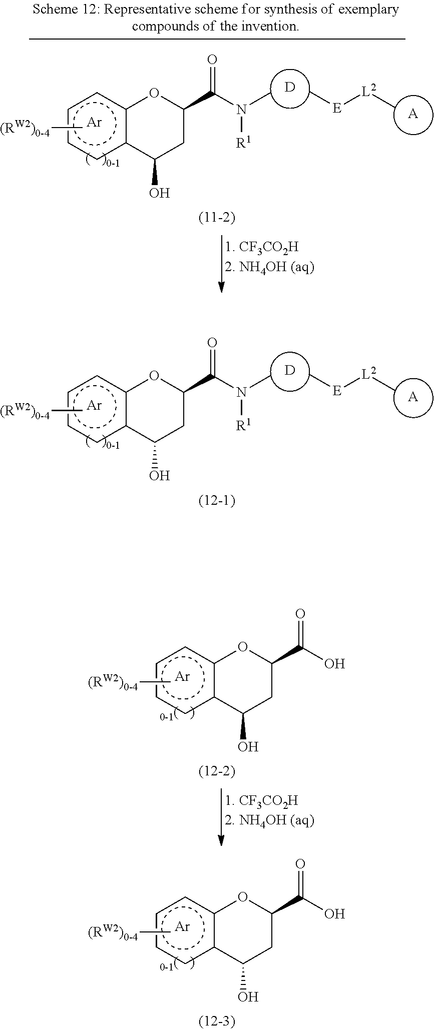

As shown in Scheme 12, compounds of formula (12-1) can be prepared from compounds of formula (11-2). Compounds of formula (11-2), wherein Ar is a fused aryl or heteroaryl ring, can be converted to compounds of formula (12-1) by treatment with optionally warmed trifluoroacetic acid for 0.5-4 hours followed by aqueous ammonium hydroxide. Similarly, compounds of formula (12-2) can be transformed to compounds of formula (12-3) under the same conditions. Compounds of formula (12-3) are intermediates to prepare compounds of Formula (I). Compounds of formula (12-1) are representative of compounds of Formula (I).

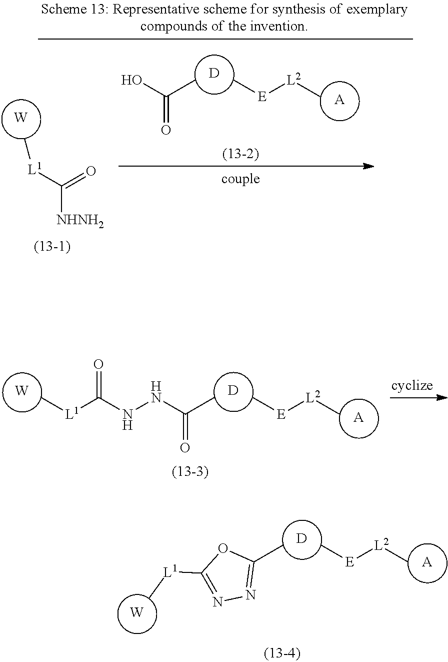

As shown in Scheme 13, compounds of formula (13-4) can be prepared from compounds of formula (13-1). Compounds of formula (13-1), can be coupled with carboxylic acids of formula (13-2) under the amide bond forming conditions described in Scheme 1 to give compounds of formula (13-3). Compounds of formula (13-3) can then be cyclized to give oxadiazoles of formula (13-4) using the conditions described in Scheme 6 or Scheme 2-3. Compounds of formula (13-4) are representative of compounds of Formula (I).

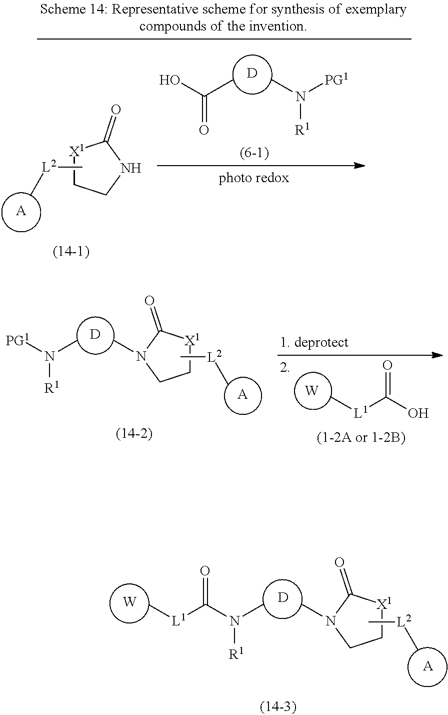

As shown in Scheme 14, compounds of formula (14-3) can be prepared from compounds of formula (14-1). Compounds of formula (14-1), wherein X is O, NH, or CH/CH₂, can be reacted with compounds of formula (6-1) under photo redox conditions to give compounds of formula (14-2). Compounds of formula (14-2) can be deprotected and then coupled with compounds of formula (1-2A) or alternatively compounds of formula (1-2B) under the amide bond forming conditions described in Scheme 1 to give compounds of formula (14-3). Compounds of formula (14-3) are representative of compounds of Formula (I).

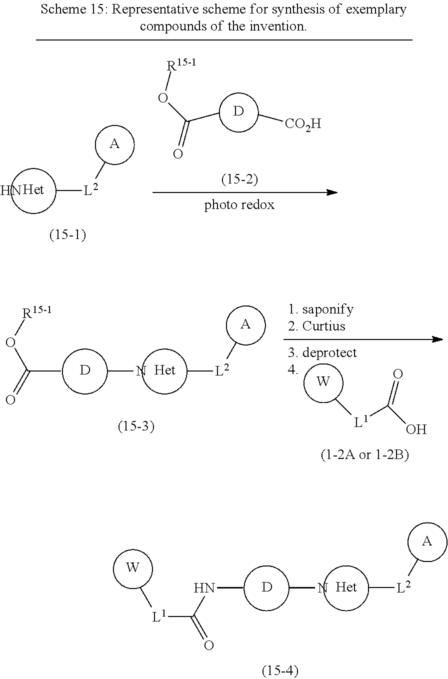

As shown in Scheme 15, compounds of formula (15-4) can be prepared from compounds of formula (15-1). Compounds of formula (15-1), wherein Het is a heteroaryl or heterocycle containing an NH moiety, can be reacted with compounds of formula (15-2), wherein R¹⁵⁻¹ is methyl or ethyl, under photo redox conditions to give compounds of formula (15-3). Compounds of formula (15-3) can be converted to compounds of formula (15-4) in a four-step process. Step one is saponification of the ester of compounds of formula (15-3) followed by the second step, a Curtius rearrangement reaction. Removal of the amine protecting group installed with the Curtius is the third step followed by coupling with compounds of formula 1-2A or 1-2B in a fourth step completes the sequence. Compounds of formula (15-4) are representative of compounds of Formula (I).

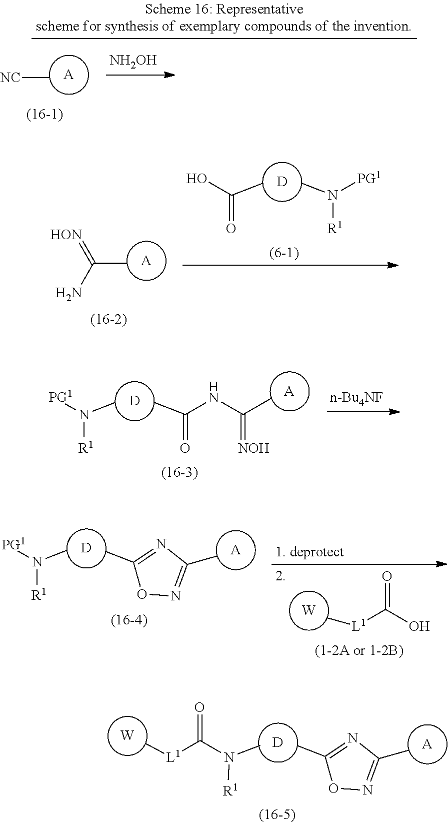

As shown in Scheme 16, compounds of formula (16-5) can be prepared from compounds of formula (16-1). Compounds of formula (16-1) can be treated with hydroxyamine to give compounds of formula (16-2). Compounds of formula (16-2) can be coupled with compounds of formula (6-1) under the amide bond forming conditions described in Scheme 1 to give compounds of formula (16-3). Compounds of formula (16-3) can be treated with tetrabutylammonium fluoride to give compounds of formula (16-4). Oxadiazoles of formula (16-4) can be deprotected and then coupled with compounds of formula (1-2A) or formula (1-2B) to give compounds of formula (16-5). Compounds of formula (16-5) are representative of compounds of Formula (I).

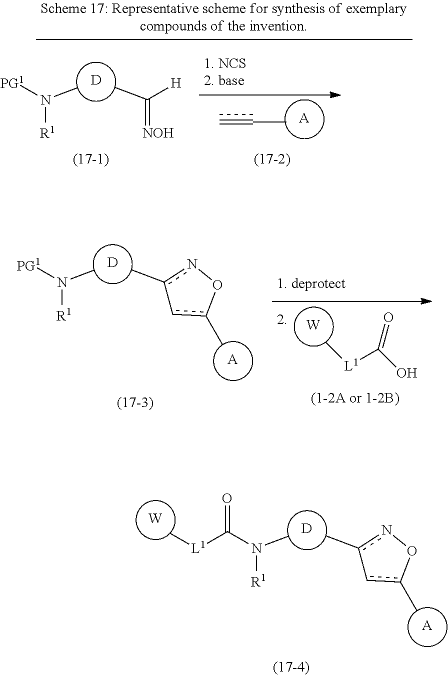

As shown in Scheme 17, compounds of formula (17-4) can be prepared from compounds of formula (17-1). Compounds of formula (17-1) can be treated with N-chlorosuccinimide. Subsequent treatment with an alkene or alkyne of formula (17-2) in the presence of a base such as triethylamine gives compounds of formula (17-3). Oxazolines or oxazoles of formula (17-3) can be deprotected and then coupled with compounds of formula (1-2A) or compounds of formula (1-2B) under conditions previously described to give compounds of formula (17-4). Compounds of formula (17-4) are representative of compounds of Formula (I).

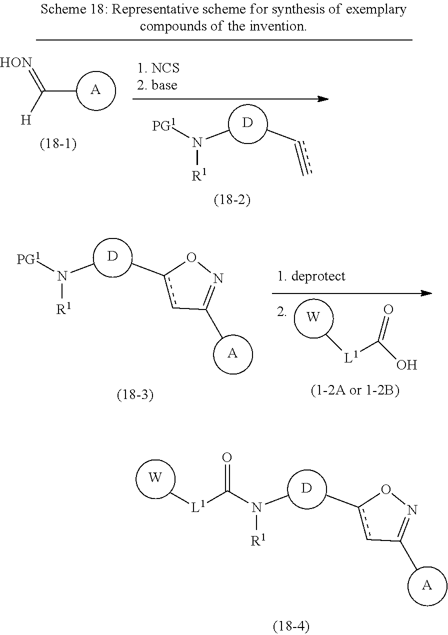

As shown in Scheme 18, compounds of formula (18-4) can be prepared from compounds of formula (18-1). Compounds of formula (18-1) can be treated with N-chlorosuccinimide. Subsequent treatment with an alkene or alkyne of formula (18-2) in the presence of a base such as triethylamine gives compounds of formula (18-3). Oxazolines or oxazoles of formula (18-3) can be deprotected and then coupled with compounds of formula (1-2A) or compounds of formula (1-2B) under conditions previously described to give compounds of formula (18-4). Compounds of formula (18-4) are representative of compounds of Formula (I).

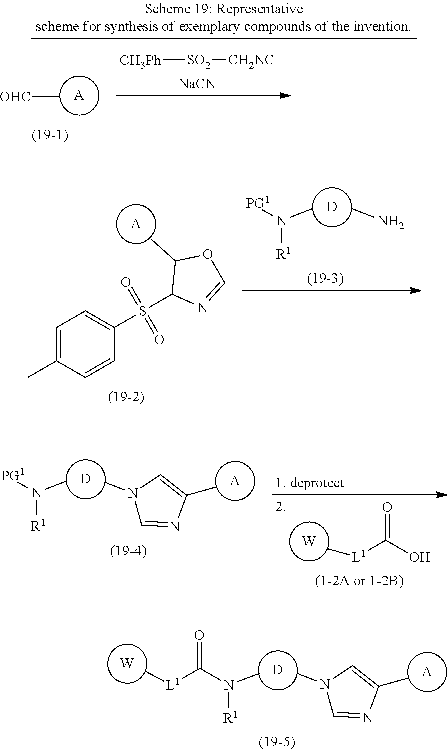

As shown in Scheme 19, compounds of formula (19-5) can be prepared from compounds of formula (19-1). Compounds of formula (19-1) can be treated with 1-((isocyanomethyl)sulfonyl)-4-methylbenzene and sodium cyanide to give compounds of formula (19-2). Compounds of formula (19-2) can be reacted with compounds of formula (19-3) in heated xylene to give compounds of formula (19-4). Compounds of formula (19-4) can be deprotected and then coupled with compounds of formula (1-2A) or compounds of formula (1-2B) under conditions previously described to give compounds of formula (19-5). Compounds of formula (19-5) are representative of compounds of Formula (I).

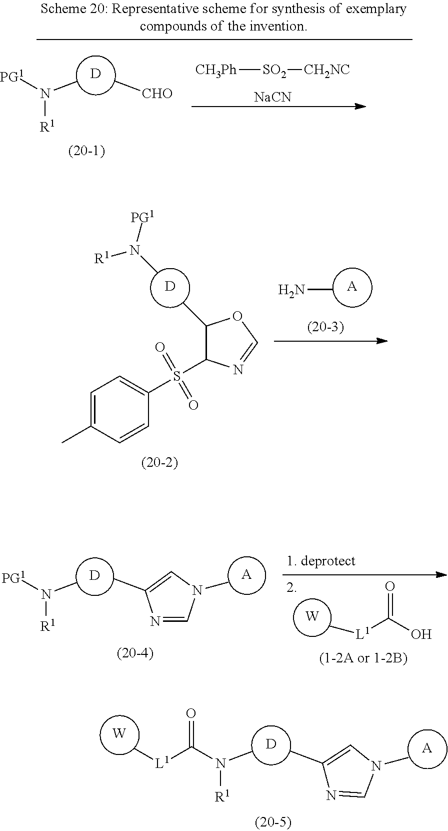

As shown in Scheme 20, compounds of formula (20-5) can be prepared from compounds of formula (20-1). Compounds of formula (20-1) can be treated with 1-((isocyanomethyl)sulfonyl)-4-methylbenzene and sodium cyanide to give compounds of formula (20-2). Compounds of formula (20-2) can be reacted with compounds of formula (20-3) in heated xylene to give compounds of formula (20-4). Compounds of formula (20-4) can be deprotected and then coupled with compounds of formula (1-2A) or compounds of formula (1-2B) under conditions previously described to give compounds of formula (20-5). Compounds of formula (20-5) are representative of compounds of Formula (I).

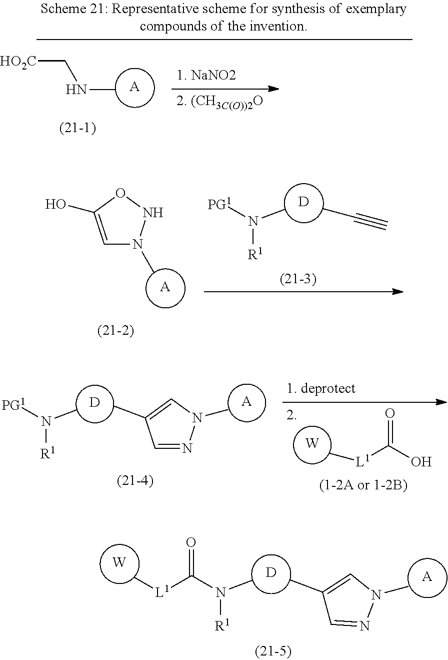

As shown in Scheme 21, compounds of formula (21-5) can be prepared from compounds of formula (21-1). Compounds of formula (21-1) can be treated with sodium nitrite and then cyclized in the presence of heated acetic anhydride to give compounds of formula (21-2). Compounds of formula (21-2) can be reacted with compounds of formula (21-3) in the presence 4,7-diphenyl-1,10-phenanthroline, copper(II) sulfate, and a base such as triethylamine to give compounds of formula (21-4). Compounds of formula (21-4) can be deprotected and then coupled with compounds of formula (1-2A) or compounds of formula (1-2B) under conditions previously described to give compounds of formula (21-5). Compounds of formula (21-5) are representative of compounds of Formula (I).

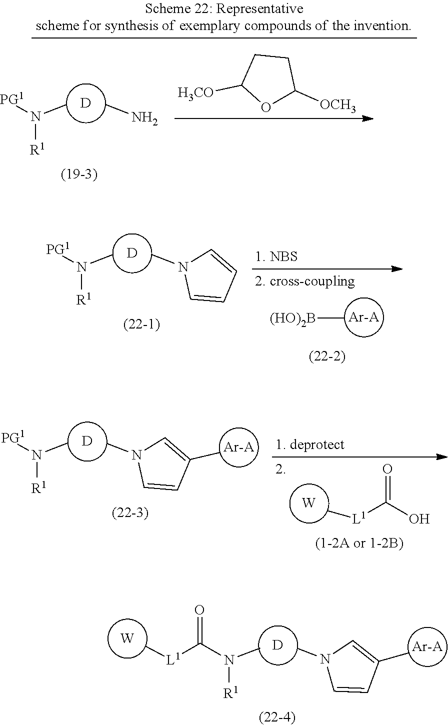

As shown in Scheme 22, compounds of formula (22-4) can be prepared from compounds of formula (19-3). Compounds of formula (19-3) can be treated with 2,5-dimethoxytetrahydrofuran in a heated mixture of acetic acid and water to give compounds of formula (22-1). Compounds of formula (22-1) can be brominated with N-bromosuccinimide (NBS) and then cross-coupled under Suzuki reaction conditions with a boronic acid or other suitable coupling partner of formula (22-2), where Ar-A is an A-ring consisting of an optionally substituted aryl or optionally substituted heteroaryl moiety, to give compounds of formula (22-3). Compounds of formula (22-3) can be deprotected and then coupled with compounds of formula (1-2A) or compounds of formula (1-2B) under conditions previously described to give compounds of formula (22-4). Compounds of formula (22-4) are representative of compounds of Formula (I).

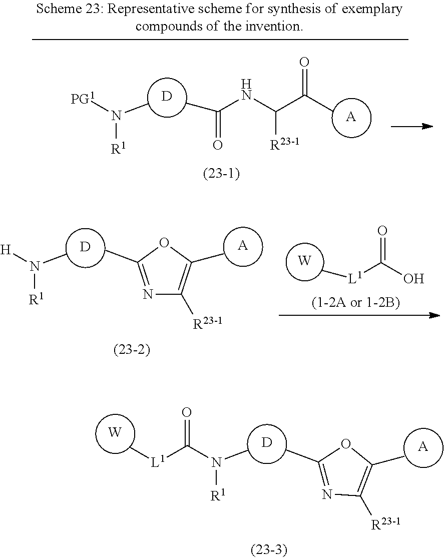

As shown in Scheme 23, compounds of formula (23-3) can be prepared from compounds of formula (23-1). Compounds of formula (23-1), wherein R²³-1 is hydrogen or methyl, can be treated with heated sulfuric acid or phosphorus oxychloride to both cyclize the starting material and remove the protecting group, PG¹, to give compounds of formula (23-2). Compounds of formula (23-2) can be coupled with compounds of formula (1-2A) or compounds of formula (1-2B) under conditions previously described to give compounds of formula (23-3). Compounds of formula (23-3) are representative of compounds of Formula (I).

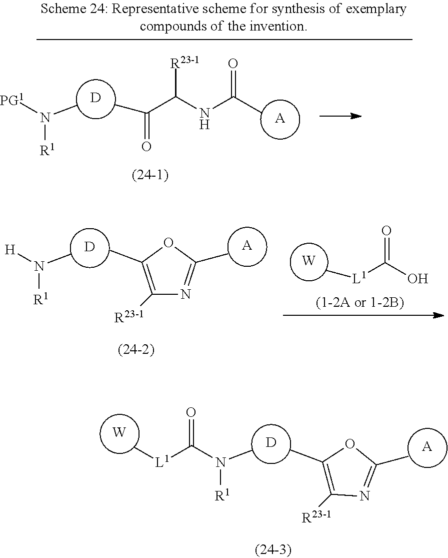

As shown in Scheme 24, compounds of formula (24-3) can be prepared from compounds of formula (24-1). Compounds of formula (24-1), wherein R²³⁻¹ is hydrogen or methyl, can be treated with heated sulfuric acid to both cyclize the starting material and remove the protecting group, PG¹, to give compounds of formula (24-2). Compounds of formula (24-2) can be coupled with compounds of formula (1-2A) or compounds of formula (1-2B) under conditions previously described to give compounds of formula (24-3). Compounds of formula (24-3) are representative of compounds of Formula (I).

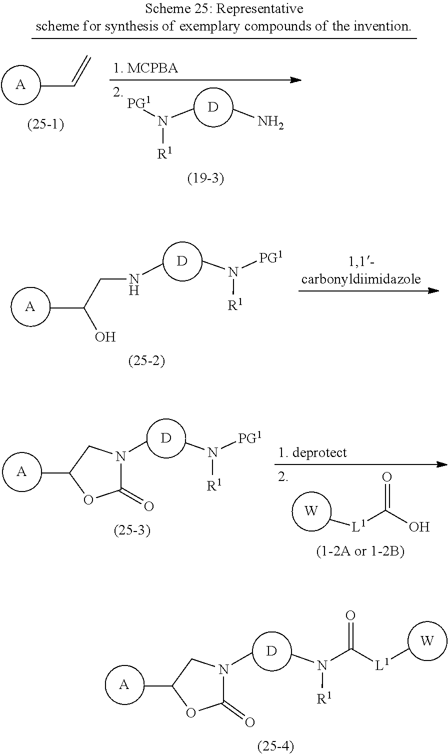

As shown in Scheme 25, compounds of formula (25-4) can be prepared from compounds of formula (25-1). Compounds of formula (25-1) can be oxidized with m-chloroperoxybenzoic acid to give an intermediate epoxide that is opened by treatment with compounds of formula (19-3) to give compounds of formula (25-2). Compounds of formula (25-2) can be reacted with 1,1′-carbonyldiimidazole to give compounds of formula (25-3). Compounds of formula (25-3) can be deprotected and then coupled with compounds of formula (1-2A) or compounds of formula (1-2B) under conditions previously described to give compounds of formula (25-4). Compounds of formula (25-4) are representative of compounds of Formula (I).

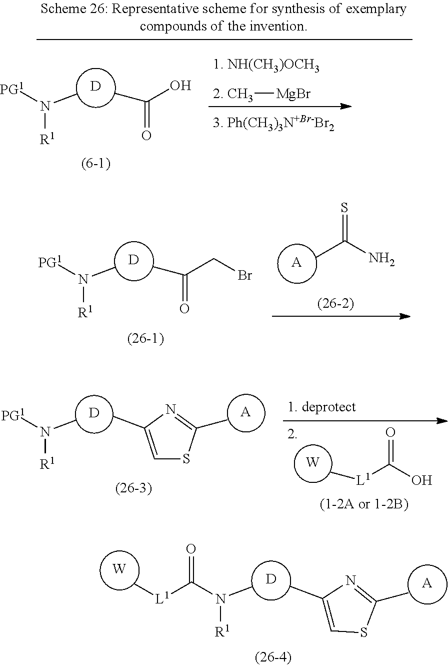

As shown in Scheme 26, compounds of formula (26-4) can be prepared from compounds of formula (6-1). Compounds of formula (6-1) can be converted to compounds of formula (26-1) in a three-step process. In the first step, compounds of formula (6-1) are coupled with N,O-dimethylhydroxylamine using an amide bond forming reaction condition described in Scheme 1. The resultant N-methoxy-N-(methyl)amide moiety is reacted in a second step with methyl magnesium bromide to give a methyl ketone. In the third step, the methyl ketone can be brominated with phenyltrimethylammonium tribromide to give compounds of formula (26-1). Compounds of formula (26-1) can be reacted with a thioamide of formula (26-2) to give compounds of formula (26-3). Compounds of formula (26-3) can be deprotected and then coupled with compounds of formula (1-2A) or compounds of formula (1-2B) under conditions previously described to give compounds of formula (26-4). Compounds of formula (26-4) are representative of compounds of Formula (I).

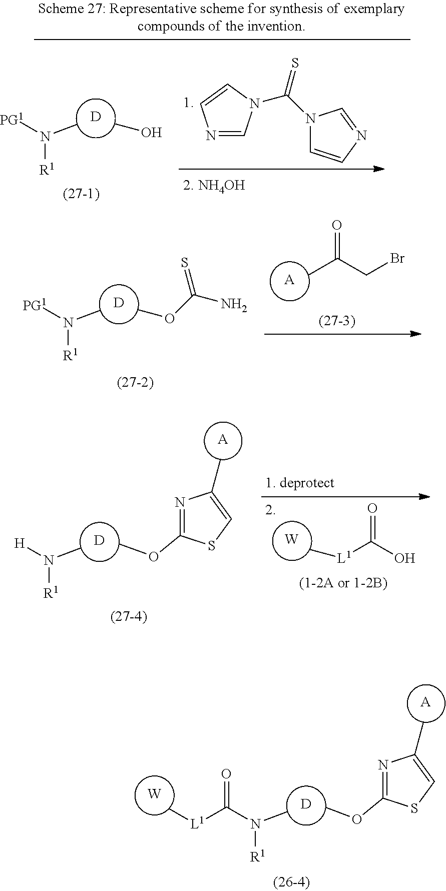

As shown in Scheme 27, compounds of formula (27-1) can be transformed to compounds of formula (27-5). Compounds of formula (27-1) can be reacted with di(1H-imidazol-1-yl)methanethione in the presence of N,N-dimethylpyridin-4-amine followed by ammonium hydroxide to give compounds of formula (27-2). Compounds of formula (27-2) can be reacted with compounds of formula (27-3) in the presence of a tertiary amine base to give compounds of formula (27-4). Compounds of formula (27-4) can be deprotected and then coupled with compounds of formula (1-2A) or compounds of formula (1-2B) under conditions previously described to give compounds of formula (27-5). Compounds of formula (27-5) are representative of compounds of Formula (I).

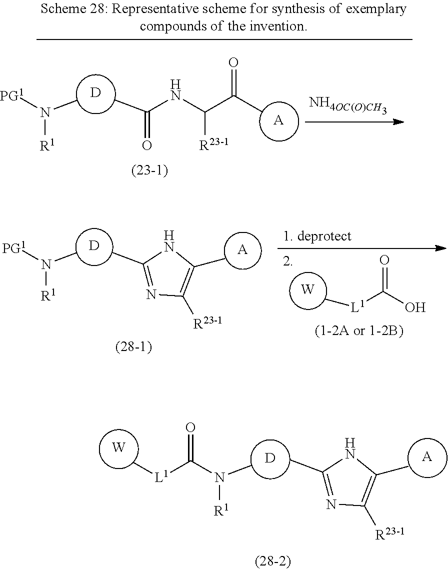

As shown in Scheme 28, compounds of formula (23-1) can be converted to compounds of formula (28-2). Compounds of formula (23-1) can be reacted with ammonium acetate in heated xylene to give compounds of formula (28-1). Compounds of formula (28-1) can be deprotected and then coupled with compounds of formula (1-2A) or compounds of formula (1-2B) under conditions previously described to give compounds of formula (28-2). Compounds of formula (28-2) are representative of compounds of Formula (I).

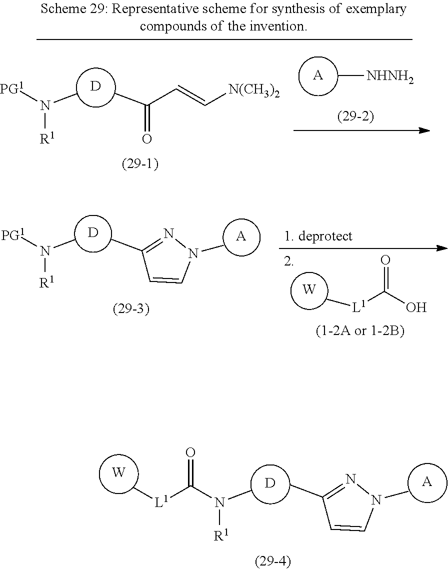

As shown in Scheme 29, compounds of formula (29-1) can be converted to compounds of formula (29-4). Compounds of formula (29-1) can be reacted with hydrazines of formula (29-2) in a solvent such as warmed methanol or ethanol to give compounds of formula (29-3). Compounds of formula (29-3) can be deprotected and then coupled with compounds of formula (1-2A) or compounds of formula (1-2B) under conditions previously described to give compounds of formula (29-4). Compounds of formula (29-4) are representative of compounds of Formula (I).

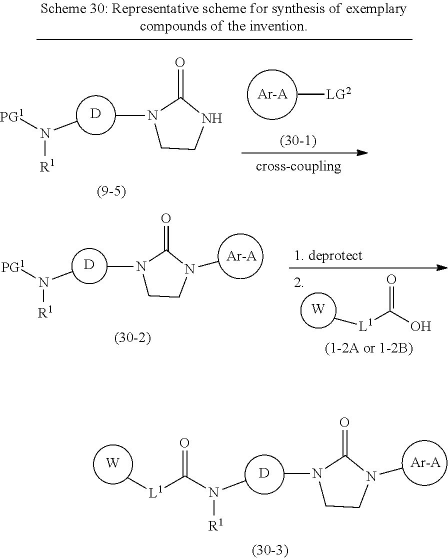

As shown in Scheme 30, compounds of formula (9-5) can be converted to compounds of formula (30-3). Compounds of formula (9-5) can be reacted with compounds of formula (30-1), wherein LG² is a leaving group such as chlorine, bromine, iodine or sulfonate and Ar-A is an A-ring consisting of an optionally substituted aryl or optionally substituted heteroaryl moiety, under palladium-mediated cross-coupling reaction conditions to give compounds of formula (30-2). Compounds of formula (30-2) can be deprotected and then coupled with compounds of formula (1-2A) or compounds of formula (1-2B) under conditions previously described to give compounds of formula (30-3). Compounds of formula (30-3) are representative of compounds of Formula (I).

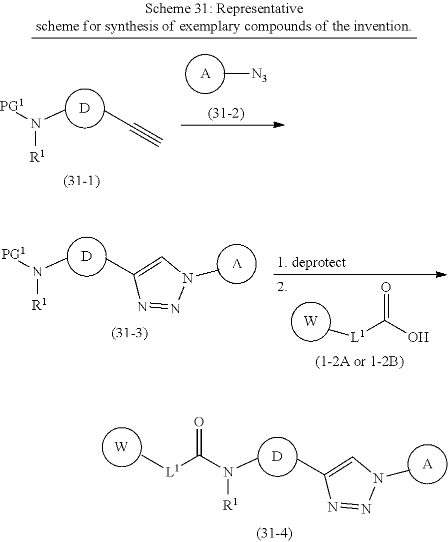

As shown in Scheme 31, compounds of formula (31-1) can be converted to compounds of formula (31-4). Compounds of formula (31-1) can be reacted with azides of formula (31-2) under click chemistry reaction conditions to give compounds of formula (31-3). Compounds of formula (31-3) can be deprotected and then coupled with compounds of formula (1-2A) or compounds of formula (1-2B) under conditions previously described to give compounds of formula (31-4). Compounds of formula (31-4) are representative of compounds of Formula (I).

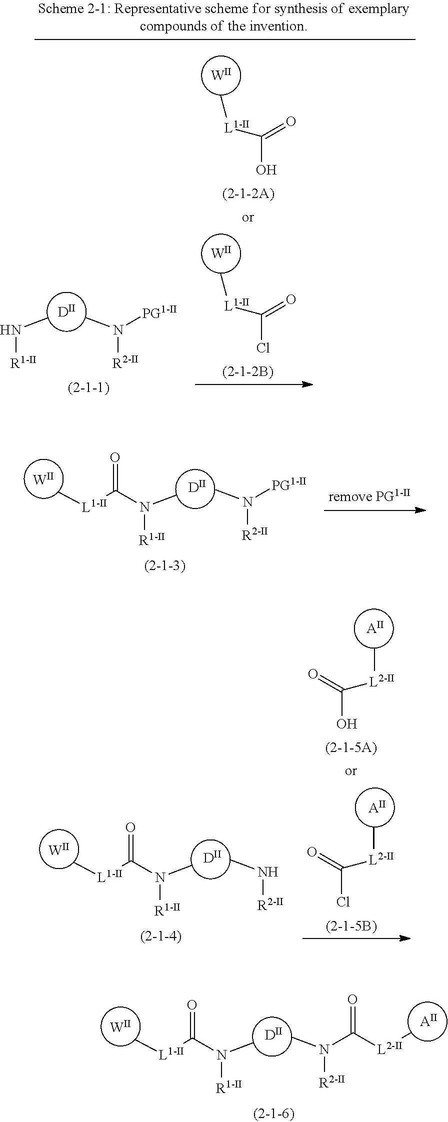

As shown in Scheme 2-1, compounds of formula (2-1-6) can be prepared from compounds of formula (2-1-1). Compounds of formula (2-1-1) where PG^{1-II} is an amine protecting group (e.g. tert-butoxycarbonyl or benzyloxycarbonyl) can be coupled with carboxylic acids of formula (2-1-2A) or alternatively with acid chlorides of formula (2-1-2B) under amide bond forming conditions to give amides of formula (2-1-3). Examples of conditions known to generate amides from a mixture of a carboxylic acid of formula (2-1-2A) and an amine of formula (2-1-1) are described in Scheme 1.

Alternatively, carboxylic acids of formula (2-1-2A) can be converted to the corresponding acid chlorides of formula (2-1-2B) by reactions described in Scheme 1. The resultant acid chlorides of formula (2-1-2B) can then be coupled with amines of formula (2-1-1) optionally in the presence of a base such as a tertiary amine base such as triethylamine or diisopropylethylamine or an aromatic base such as pyridine, at room temperature in a solvent such as dichloromethane to give amides of formula (2-1-3).

Compounds of formula (2-1-3) can be deprotected using conditions known to one of skill in the art and dependent upon the protecting group (PG^{1-II}) used to give compounds of formula (2-1-4). Compounds of formula (2-1-4) can be coupled with carboxylic acids of formula (2-1-5A) or alternatively acid chlorides of formula (2-1-5B) under amide bond forming conditions as discussed above to afford compounds of formula (2-1-6). Compounds of formula (2-1-6) are representative compounds of Formula (II).

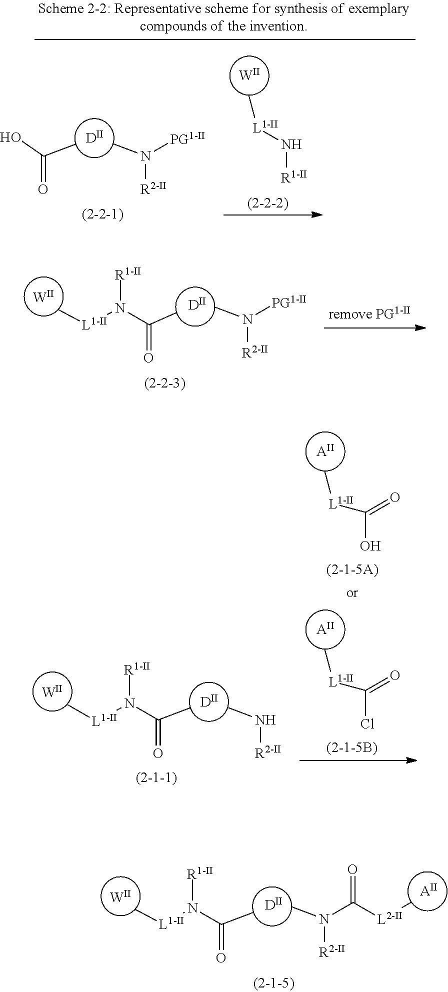

Scheme 2-2: As shown in Scheme 2-2, compounds of formula (2-2-5) can be prepared from compounds of formula (2-2-1). Compounds of formula (2-2-1) where PG^{1-II} is an amine protecting group (e.g. tert-butoxycarbonyl or benzyloxycarbonyl) can be coupled with amines of formula (2-2-2) under amide bond forming conditions to give amides of formula (2-2-3). Examples of conditions known to generate amides from a mixture of a carboxylic acid of formula (2-2-1) and an amine of formula (2-2-2) are described in Scheme 1.

Compounds of formula (2-2-3) can be deprotected using conditions known to one of skill in the art and dependent upon the protecting group (PG^{1-II}) used to give compounds of formula (2-2-4). Compounds of formula (2-2-4) can be coupled with carboxylic acids of formula (2-1-5A) or alternatively acid chlorides of formula (2-1-5B) under amide bond forming conditions as discussed above to afford compounds of formula (2-2-5). Compounds of formula (2-2-5) are representative compounds of Formula (II).

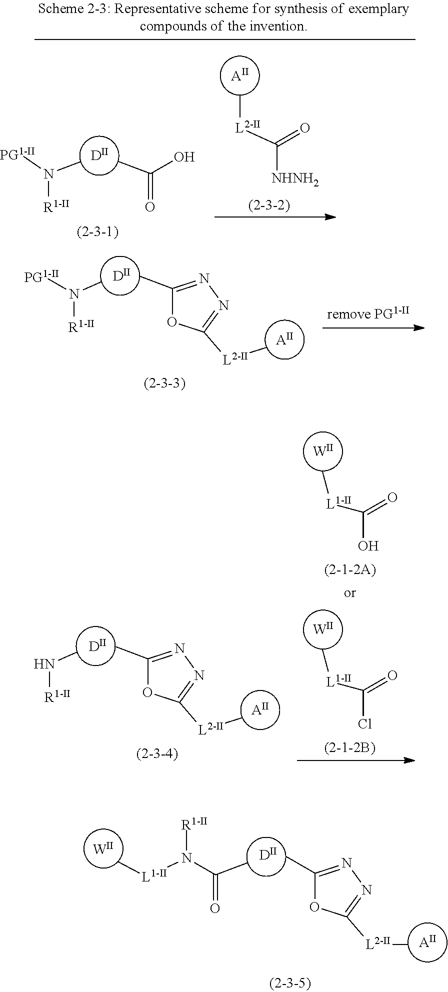

As shown in Scheme 2-3, compounds of formula (2-3-1) can be reacted with compounds of formula (2-3-2) in heated phosphorus oxychloride to give compounds of formula (2-3-3). Alternatively, compounds of formula (2-3-1) can also be reacted with compounds of formula (2-3-2) under the amide bond coupling conditions described to make compounds of formula (1-3). Following the coupling, the intermediate can be cyclized and dehydrated using 4-methylbenzene-1-sulfonyl chloride in the presence of a tertiary amine base such as N,N-diisopropylethylamine in heated acetonitrile to give compounds of formula (2-3-3). Compounds of formula (2-3-3) can be deprotected using conditions known to one of skill in the art and dependent upon the protecting group (PG^{1-II}) used to give compounds of formula (2-3-4). Compounds of formula (2-3-4) can be coupled with carboxylic acids of formula (2-1-2A) or alternatively acid chlorides of formula (2-1-2B) under amide bond forming conditions as discussed above to afford compounds of formula (2-3-5). Compounds of formula (2-3-5) are representative compounds of Formula (II).

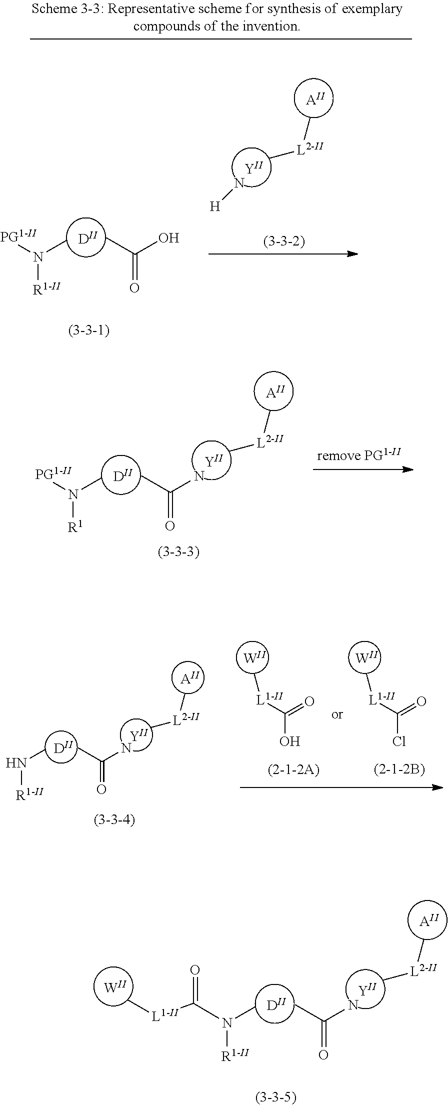

As shown in Scheme 3-3, compounds of formula (3-3-5) can be prepared from compounds of formula (3-3-1). Compounds of formula (3-3-1) where PG^{1-II} is an amine protecting group (e.g. tert-butoxycarbonyl or benzyloxycarbonyl) can be coupled with amines of formula (3-3-2) under amide bond forming conditions to give amides of formula (3-3-3). Examples of conditions known to generate amides from a mixture of a carboxylic acid of formula (3-3-1) and an amine of formula (3-3-2) are described in Scheme 1.

Compounds of formula (3-3-3) can be deprotected using conditions known to one of skill in the art and dependent upon the protecting group (PG^{1-II}) used to give compounds of formula (3-3-4). Compounds of formula (3-3-4) can be coupled with carboxylic acids of formula (2-1-5A) or alternatively acid chlorides of formula (2-1-5B) under amide bond forming conditions as discussed above to afford compounds of formula (3-3-5). Compounds of formula (3-3-5) are representative compounds of Formula (II).

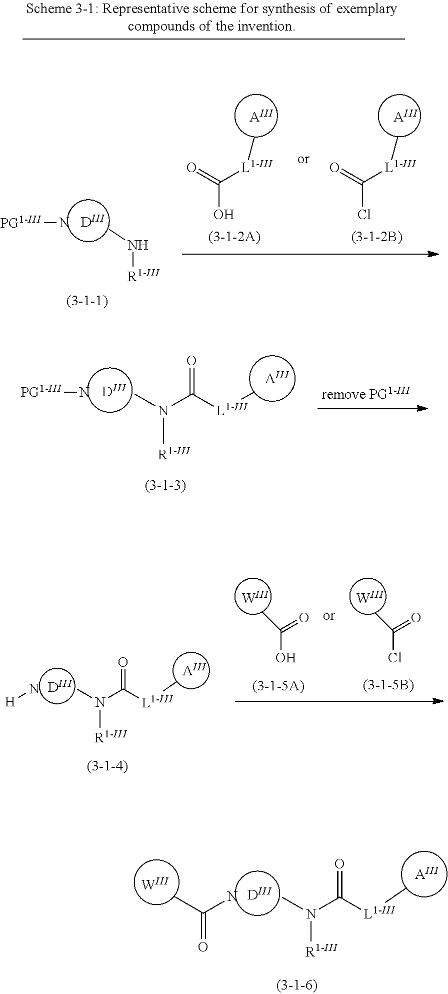

As shown in Scheme 3-1, compounds of formula (3-1-6) can be prepared from compounds of formula (3-1-1). Compounds of formula (3-1-1) where PG^{1-III} is an amine protecting group (e.g. tert-butoxycarbonyl or benzyloxycarbonyl) can be coupled with carboxylic acids of formula (3-1-2A) or alternatively with acid chlorides of formula (3-1-2B) under amide bond forming conditions to give amides of formula (3-1-3). Examples of conditions known to generate amides from a mixture of a carboxylic acid of formula (3-1-2A) and an amine of formula (3-1-1) are described in Scheme 1.

Alternatively, carboxylic acids of formula (3-1-2A) can be converted to the corresponding acid chlorides of formula (3-1-2B) by reactions described in Scheme 1. The resultant acid chlorides of formula (3-1-2B) can then be coupled with amines of formula (3-1-1) optionally in the presence of a base such as a tertiary amine base such as triethylamine or diisopropylethylamine or an aromatic base such as pyridine, at room temperature in a solvent such as dichloromethane to give amides of formula (3-1-3).

Compounds of formula (3-1-3) can be deprotected using conditions known to one of skill in the art and dependent upon the protecting group (PG^{1-III}) used to give compounds of formula (3-1-4). Compounds of formula (3-14) can be coupled with carboxylic acids of formula (3-1-5A) or alternatively acid chlorides of formula (3-1-5B) under amide bond forming conditions as discussed above to afford compounds of formula (3-1-6). Compounds of formula (3-1-6) are representative compounds of Formula (III-a).

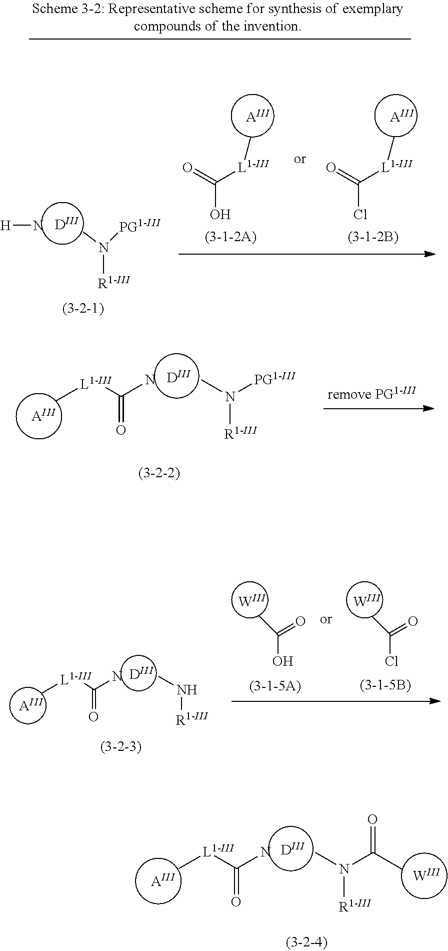

As shown in Scheme 3-2, compounds of formula (3-2-4) can be prepared from compounds of formula (3-2-1). Compounds of formula (3-2-1) where PG^{1-III} is an amine protecting group (e.g. tert-butoxycarbonyl or benzyloxycarbonyl) can be coupled with carboxylic acids of formula (3-1-2A) or alternatively with acid chlorides of formula (3-1-2B) under amide bond forming conditions to give amides of formula (3-2-2). Examples of conditions known to generate amides from a mixture of a carboxylic acid of formula (3-1-2A) and an amine of formula (3-2-1) are described in Scheme 1.

Alternatively, carboxylic acids of formula (3-1-2A) can be converted to the corresponding acid chlorides of formula (3-1-2B) by reactions described in Scheme 1. The resultant acid chlorides of formula (3-1-2B) can then be coupled with amines of formula (3-2-1) optionally in the presence of a base such as a tertiary amine base such as triethylamine or diisopropylethylamine or an aromatic base such as pyridine, at room temperature in a solvent such as dichloromethane to give amides of formula (3-2-2).

Compounds of formula (3-2-2) can be deprotected using conditions known to one of skill in the art and dependent upon the protecting group (PG^{1-III}) used to give compounds of formula (3-2-3). Compounds of formula (3-2-3) can be coupled with carboxylic acids of formula (3-1-5A) or alternatively acid chlorides of formula (3-1-5B) under amide bond forming conditions as discussed above to afford compounds of formula (3-2-4). Compounds of formula (3-24) are representative compounds of Formula (III-b).

### Pharmaceutical Compositions

The present invention features pharmaceutical compositions comprising a compound of Formula (I), Formula (II), Formula (III-a) or Formula (III-b), or a pharmaceutically acceptable salt, solvate, hydrate, tautomer, or stereoisomer thereof. In some embodiments, the pharmaceutical composition further comprises a pharmaceutically acceptable excipient. In some embodiments, the compound of Formula (I), Formula (II), Formula (III-a) or Formula (III-b) or a pharmaceutically acceptable salt, solvate, hydrate, tautomer, stereoisomer thereof is provided in an effective amount in the pharmaceutical composition. In some embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), Formula (II), Formula (III-a) or Formula (III-b), or a pharmaceutically acceptable salt, solvate, hydrate, tautomer, stereoisomer thereof (the “active ingredient”) into association with a carrier and/or one or more other accessory ingredients, and then, if necessary and/or desirable, shaping and/or packaging the product into a desired single- or multi-dose unit. Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of a compound of Formula (I), Formula (II), Formula (III-a) or Formula (III-b), the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) of a compound of Formula (I), Formula (II), Formula (III-a) or Formula (III-b), or a pharmaceutically acceptable salt, solvate, hydrate, tautomer, stereoisomer thereof.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A compound of Formula (I), Formula (II), Formula (III-a) or Formula (III-b), or a pharmaceutically acceptable salt, solvate, hydrate, tautomer, stereoisomer thereof may also be in micro-encapsulated form.

The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212, 162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, J. Hosp. Pharm. 46: 1576-1587, 1989). The compositions of the present invention can also be delivered as nanoparticles.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

In some embodiments, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein, e.g., a compound of Formula (I), Formula (II), Formula (III-a) or Formula (III-b), or a pharmaceutically acceptable salt, solvate, hydrate, tautomer, or stereoisomer thereof are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound of Formula (I), Formula (II), Formula (III-a) or Formula (III-b), or a pharmaceutically acceptable salt, solvate, hydrate, tautomer, or stereoisomer thereof for administration one or more times a day may comprise about 0.0001 mg to about 5000 mg, e.g., from about 0.0001 mg to about 4000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound of Formula (I), Formula (II), Formula (III-a) or Formula (III-b), or a pharmaceutically acceptable salt, solvate, hydrate, tautomer, or stereoisomer thereof may be at dosage levels sufficient to deliver from about 0.001 mg/kg to about 1000 mg/kg, e.g., about 0.001 mg/kg to about 500 mg/kg, about 0.01 mg/kg to about 250 mg/kg, about 0.1 mg/kg to about 100 mg/kg, about 0.1 mg/kg to about 50 mg/kg, about 0.1 mg/kg to about 40 mg/kg, about 0.1 mg/kg to about 25 mg/kg, about 0.01 mg/kg to about 10 mg/kg, about 0.1 mg/kg to about 10 mg/kg, or about 1 mg/kg to about 50 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, e.g., a compound of Formula (I), Formula (II), Formula (III-a) or Formula (III-b), or a pharmaceutically acceptable salt, solvate, hydrate, tautomer, or stereoisomer thereof as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and pain-relieving agents. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Pharmaceutical compositions provided by the present invention include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. eIF2B, eIF2 or component of eIF2α signal transduction pathway or component of phosphorylated eIF2α pathway or the ISR pathway), and/or reducing, eliminating, or slowing the progression of disease symptoms (e.g. symptoms of cancer a neurodegenerative disease, a leukodystrophy, an inflammatory disease, a musculoskeletal disease, a metabolic disease, or a disease or disorder associated with impaired function of eIF2B, eIF2α or a component of the eIF2 pathway or ISR pathway). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. a symptom of cancer, a neurodegenerative disease, a leukodystrophy, an inflammatory disease, a musculoskeletal disease, a metabolic disease, or a disease or disorder associated with impaired function of eIF2B, eIF2 α, or a component of the eIF2 pathway or ISR pathway), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Also encompassed by the invention are kits (e.g., pharmaceutical packs). The inventive kits may be useful for preventing and/or treating a disease (e.g., cancer, a neurodegenerative disease, a leukodystrophy, an inflammatory disease, a musculoskeletal disease, a metabolic disease, or other disease or condition described herein).

The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Thus, in one aspect, provided are kits including a first container comprising a compound of Formula (I), Formula (II), Formula (III-a) or Formula (III-b), or a pharmaceutically acceptable salt, solvate, hydrate, tautomer, or stereoisomer thereof, or a pharmaceutical composition thereof. In certain embodiments, the kits are useful in preventing and/or treating a proliferative disease in a subject. In certain embodiments, the kits further include instructions for administering a compound of Formula (I), Formula (II), Formula (III-a) or Formula (III-b), or a pharmaceutically acceptable salt, solvate, hydrate, tautomer, or stereoisomer thereof, or a pharmaceutical composition thereof, to a subject to prevent and/or treat a disease described herein.

### Methods of Treatment

The present invention features compounds, compositions, and methods comprising a compound of Formula (I), Formula (II), Formula (III-a) or Formula (III-b), or a pharmaceutically acceptable salt, solvate, hydrate, tautomer, ester, N-oxide or stereoisomer thereof. In some embodiments, the compounds, compositions, and methods are used in the prevention or treatment of a disease, disorder, or condition. Exemplary diseases, disorders, or conditions include, but are not limited to a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a disease with mutations that leads to UPR induction, a malaria infection, a musculoskeletal disease, a metabolic disease, or a mitochondrial disease.

In some embodiments, the disease, disorder, or condition is related to (e.g., caused by) modulation of (e.g., a decrease in) eIF2B activity or level, eIF2α activity or level, or a component of the eIF2 pathway or ISR pathway. In some embodiments, the disease, disorder, or condition is related to modulation of a signaling pathway related to a component of the eIF2 pathway or ISR pathway (e.g., phosphorylation of a component of the eIF2 pathway or ISR pathway). In some embodiments, the disease, disorder, or condition is related to (e.g., caused by) neurodegeneration. In some embodiments, the disease, disorder, or condition is related to (e.g., caused by) neural cell death or dysfunction. In some embodiments, the disease, disorder, or condition is related to (e.g., caused by) glial cell death or dysfunction. In some embodiments, the disease, disorder, or condition is related to (e.g., caused by) an increase in the level or activity of eIF2B, eIF2α, or a component of the eIF2 pathway or ISR pathway. In some embodiments, the disease, disorder, or condition is related to (e.g., caused by) a decrease in the level or activity of eIF2B, eIF2α, or a component of the eIF2 pathway or ISR pathway.

In some embodiments, the disease may be caused by a mutation to a gene or protein sequence related to a member of the eIF2 pathway (e.g., eIF2B, eIF2α, or other component). Exemplary mutations include an amino acid mutation in the eIF2B1, eIF2B2, eIF2B3, eIF2B4, eIF2B5 subunits. In some embodiments, an amino acid mutation (e.g., an amino acid substitution, addition, or deletion) in a particular protein that may result in a structural change, e.g., a conformational or steric change, that affects the function of the protein. For example, in some embodiments, amino acids in and around the active site or close to a binding site (e.g., a phosphorylation site, small molecule binding site, or protein-binding site) may be mutated such that the activity of the protein is impacted. In some instances, the amino acid mutation (e.g., an amino acid substitution, addition, or deletion) may be conservative and may not substantially impact the structure or function of a protein. For example, in certain cases, the substitution of a serine residue with a threonine residue may not significantly impact the function of a protein. In other cases, the amino acid mutation may be more dramatic, such as the substitution of a charged amino acid (e.g., aspartic acid or lysine) with a large, nonpolar amino acid (e.g., phenylalanine or tryptophan) and therefore may have a substantial impact on protein function. The nature of the mutations that affect the structure of function of a gene or protein may be readily identified using standard sequencing techniques, e.g., deep sequencing techniques that are well known in the art. In some embodiments, a mutation in a member of the eIF2 pathway may affect binding or activity of a compound of Formula (I), Formula (II), Formula (III-a) or Formula (III-b), or a pharmaceutically acceptable salt, solvate, hydrate, tautomer, ester, N-oxide or stereoisomer thereof and thereby modulate treatment of a particular disease, disorder, or condition, or a symptom thereof.

In some embodiments, an eIF2 protein may comprise an amino acid mutation (e.g., an amino acid substitution, addition, or deletion) at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid substitution at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid addition at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid deletion at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

In some embodiments, the eIF2 protein may comprise an amino acid mutation (e.g., an amino acid substitution, addition, or deletion) at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid substitution at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid addition at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid deletion at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. Exemplary mutations include V183F (eIF2B1 subunit), H341Q (eIF2B3), I346T (eIF2B3), R483W (eIF2B4), R113H (eIF2B5), and R195H (eIF2B5).

In some embodiments, an amino acid mutation (e.g., an amino acid substitution, addition, or deletion) in a member of the eIF2 pathway (e.g., an eIF2B protein subunit) may affect binding or activity of a compound of Formula (I), Formula (II), Formula (III-a) or Formula (III-b), or a pharmaceutically acceptable salt, solvate, hydrate, tautomer, ester, N-oxide or stereoisomer thereof and thereby modulate treatment of a particular disease, disorder, or condition, or a symptom thereof.

### Neurodegenerative Disease

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a neurodegenerative disease. As used herein, the term “neurodegenerative disease” refers to a disease or condition in which the function of a subject's nervous system becomes impaired. Examples of a neurodegenerative disease that may be treated with a compound, pharmaceutical composition, or method described herein include Alexander's disease, Alper's disease, Alzheimer's disease, Amyotrophic lateral sclerosis (ALS), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Dystonia, frontotemporal dementia (FTD), Gerstmann-Straussler-Scheinker syndrome, Huntington's disease, HIV-associated dementia, Kennedy's disease, Krabbe disease, kuru, Lewy body dementia, Machado-Joseph disease (Spinocerebellar ataxia type 3), Multiple system atrophy, Multisystem proteinopathy, Narcolepsy, Neuroborreliosis, Parkinson's disease, Pelizaeus-Merzbacher Disease, Pick's disease, Primary lateral sclerosis, Prion diseases, Refsum's disease, Sandhoff disease, Schilder's disease, Subacute combined degeneration of spinal cord secondary to Pernicious Anaemia, Schizophrenia, Spinocerebellar ataxia (multiple types with varying characteristics, e.g., Spinocerebellar ataxia type 2 or Spinocerebellar ataxia type 8), Spinal muscular atrophy, Steele-Richardson-Olszewski disease, progressive supranuclear palsy, corticobasal degeneration, adrenoleukodystrophy, X-linked adrenoleukodystrophy, cerebral adrenoleukodystrophy, Pelizaeus-Merzbacher Disease, Krabbe disease, leukodystrophy due to mutation in DARS2 gene (sometimes known as leukoencephalopathy with brainstem and spinal cord involvement and lactate elevation (LBSL), DARS2-related spectrum disorders, or Tabes dorsalis.

In some embodiments, the neurodegenerative disease comprises vanishing white matter disease, childhood ataxia with CNS hypo-myelination, a leukodystrophy, a leukoencephalopathy, a hypomyelinating or demyelinating disease, an intellectual disability syndrome (e.g., Fragile X syndrome), Alzheimer's disease, amyotrophic lateral sclerosis (ALS), Creutzfeldt-Jakob disease, frontotemporal dementia (FTD), Gerstmann-Straussler-Scheinker disease, Huntington's disease, dementia (e.g., HIV-associated dementia or Lewy body dementia), kuru, multiple sclerosis, Parkinson's disease, or a prion disease.

In some embodiments, the neurodegenerative disease comprises vanishing white matter disease, childhood ataxia with CNS hypo-myelination, a leukodystrophy, a leukoencephalopathy, a hypomyelinating or demyelinating disease, or an intellectual disability syndrome (e.g., Fragile X syndrome).

In some embodiments, the neurodegenerative disease comprises a psychiatric disease such as agoraphobia, Alzheimer's disease, anorexia nervosa, amnesia, anxiety disorder, attention deficit disorder, bipolar disorder, body dysmorphic disorder, bulimia nervosa, claustrophobia, depression, delusions, Diogenes syndrome, dyspraxia, insomnia, Munchausen's syndrome, narcolepsy, narcissistic personality disorder, obsessive-compulsive disorder, psychosis, phobic disorder, schizophrenia, seasonal affective disorder, schizoid personality disorder, sleepwalking, social phobia, substance abuse, tardive dyskinesia, Tourette syndrome, or trichotillomania.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat vanishing white matter disease. Exemplary methods of treating vanishing white matter disease include, but are not limited to, reducing or eliminating a symptom of vanishing white matter disease, reducing the loss of white matter, reducing the loss of myelin, increasing the amount of myelin, or increasing the amount of white matter in a subject.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat childhood ataxia with CNS hypo-myelination. Exemplary methods of treating childhood ataxia with CNS hypo-myelination include, but are not limited to, reducing or eliminating a symptom of childhood ataxia with CNS hypo-myelination, increasing the level of myelin, or decreasing the loss of myelin in a subject.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat an intellectual disability syndrome (e.g., Fragile X syndrome). Exemplary methods of treating an intellectual disability syndrome include, but are not limited to, reducing or eliminating a symptom of an intellectual disability syndrome.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat neurodegeneration. Exemplary methods of treating neurodegeneration include, but are not limited to, improvement of mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a leukoencephalopathy or demyelinating disease. Exemplary leukoencephalopathies include, but are not limited to, progressive multifocal leukoencephalopathy, toxic leukoencephalopathy, leukoencephalopathy with vanishing white matter, leukoencephalopathy with neuroaxonal spheroids, reversible posterior leukoencephalopathy syndrome, hypertensive leukoencephalopathy, megalencephalic leukoencephalopathy with subcortical cysts, Charcot-Marie-Tooth disorder, and Devic's disease. A leukoencephalopathy may comprise a demyelinating disease, which may be inherited or acquired. In some embodiments, an acquired demyelinating disease may be an inflammatory demyelinating disease (e.g., an infectious inflammatory demyelinating disease or a non-infectious inflammatory demyelinating disease), a toxic demyelinating disease, a metabolic demyelinating disease, a hypoxic demyelinating disease, a traumatic demyelinating disease, or an ischemic demyelinating disease (e.g., Binswanger's disease). Exemplary methods of treating a leukoencephalopathy or demyelinating disease include, but are not limited to, reducing or eliminating a symptom of a leukoencephalopathy or demyelinating disease, reducing the loss of myelin, increasing the amount of myelin, reducing the loss of white matter in a subject, or increasing the amount of white matter in a subject.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a traumatic injury or a toxin-induced injury to the nervous system (e.g., the brain). Exemplary traumatic brain injuries include, but are not limited to, a brain abscess, concussion, ischemia, brain bleeding, cranial fracture, diffuse axonal injury, locked-in syndrome, or injury relating to a traumatic force or blow to the nervous system or brain that causes damage to an organ or tissue. Exemplary toxin-induced brain injuries include, but are not limited to, toxic encephalopathy, meningitis (e.g. bacterial meningitis or viral meningitis), meningoencephalitis, encephalitis (e.g., Japanese encephalitis, eastern equine encephalitis, West Nile encephalitis), Guillan-Barre syndrome, Sydenham's chorea, rabies, leprosy, neurosyphilis, a prion disease, or exposure to a chemical (e.g., arsenic, lead, toluene, ethanol, manganese, fluoride, dichlorodiphenyltrichloroethane (DDT), dichlorodiphenyldichloroethylene (DDE), tetrachloroethylene, a polybrominated diphenyl ether, a pesticide, a sodium channel inhibitor, a potassium channel inhibitor, a chloride channel inhibitor, a calcium channel inhibitor, or a blood brain barrier inhibitor).

In other embodiments, the compound of Formula (I), Formula (II), Formula (III-a) or Formula (III-b), or a pharmaceutically acceptable salt, solvate, hydrate, tautomer, ester, N-oxide or stereoisomer thereof is used to improve memory in a subject. Induction of memory has been shown to be facilitated by decreased and impaired by increased eIF2α phosphorylation. Regulators of translation, such as compounds disclosed herein (e.g. a compound of Formula (I), Formula (II), Formula (III-a) or Formula (III-b)), could serve as therapeutic agents that improve memory in human disorders associated with memory loss such as Alzheimer's disease and in other neurological disorders that activate the UPR or ISR in neurons and thus could have negative effects on memory consolidation such as Parkinson's disease, schizophrenia, amyotrophic lateral sclerosis (ALS) and prion diseases. In addition, a mutation in eIF2γ that disrupts complex integrity linked intellectual disability (intellectual disability syndrome or ID) to impaired translation initiation in humans. Hence, two diseases with impaired eIF2 function, ID and VWM, display distinct phenotypes but both affect mainly the brain and impair learning. In some embodiments, the disease or condition is unsatisfactory memory (e.g., working memory, long term memory, short term memory, or memory consolidation).

In still other embodiments, the compound of Formula (I), Formula (II), Formula (III-a) or Formula (III-b), or a pharmaceutically acceptable salt, solvate, hydrate, tautomer, ester, N-oxide or stereoisomer thereof is used in a method to improve memory in a subject (e.g., working memory, long term memory, short term memory, or memory consolidation). In some embodiments, the subject is human. In some embodiments, the subject is a non-human mammal. In some embodiments, the subject is a domesticated animal. In some embodiments, the subject is a dog. In some embodiments, the subject is a bird. In some embodiments, the subject is a horse. In embodiments, the patient is a bovine. In some embodiments, the subject is a primate.

### Cancer

In some embodiments, the compound of Formula (I), Formula (II), Formula (III-a) or Formula (III-b), or a pharmaceutically acceptable salt, solvate, hydrate, tautomer, or stereoisomer thereof is used to treat cancer. As used herein, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma (e.g., WNT-dependent pediatric medulloblastoma), Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

In some embodiments, the compound of Formula (I), Formula (II), Formula (III-a) or Formula (III-b), or a pharmaceutically acceptable salt, solvate, hydrate, tautomer, or stereoisomer thereof is used to treat pancreatic cancer, breast cancer, multiple myeloma, cancers of secretory cells. For example certain methods herein treat cancer by decreasing or reducing or preventing the occurrence, growth, metastasis, or progression of cancer. In some embodiments, the methods described herein may be used to treat cancer by decreasing or eliminating a symptom of cancer. In some embodiments, the compound of Formula (I), Formula (II), Formula (III-a) or Formula (III-b), or a pharmaceutically acceptable salt, solvate, hydrate, tautomer, or stereoisomer thereof may be used as a single agent in a composition or in combination with another agent in a composition to treat a cancer described herein (e.g., pancreatic cancer, breast cancer, multiple myeloma, cancers of secretory cells).

In some embodiments, the compounds (compounds described herein, e.g., a compound of Formula (I), Formula (II), Formula (III-a) or Formula (III-b)) and compositions (e.g., compositions comprising a compound described herein, e.g., a compound of Formula (I), Formula (II), Formula (III-a) or Formula (III-b))) are used with a cancer immunotherapy (e.g., a checkpoint blocking antibody) to treat a subject (e.g., a human subject), e.g., suffering from a disease or disorder described herein (e.g., abnormal cell growth, e.g., cancer (e.g., a cancer described herein)). The methods described herein comprise administering a compound described herein, e.g., a compound of Formula (I), Formula (II), Formula (III-a) or Formula (III-b) and an immunotherapy to a subject having abnormal cell growth such as cancer. Exemplary immunotherapies include, but are not limited to the following.

In some embodiments, the immunotherapeutic agent is a compound (e.g., a ligand, an antibody) that inhibits the immune checkpoint blockade pathway. In some embodiments, the immunotherapeutic agent is a compound that inhibits the indoleamine 2,3-dioxygenase (IDO) pathway. In some embodiments, the immunotherapeutic agent is a compound that agonizes the STING pathway. Cancer immunotherapy refers to the use of the immune system to treat cancer. Three groups of immunotherapy used to treat cancer include cell-based, antibody-based, and cytokine therapies. All groups exploit cancer cells' display of subtly different structures (e.g., molecular structure; antigens, proteins, molecules, carbohydrates) on their surface that can be detected by the immune system. Cancer immunotherapy (i.e., anti-tumor immunotherapy or anti-tumor immunotherapeutics) includes but is not limited to, immune checkpoint antibodies (e.g., PD-1 antibodies, PD-L1 antibodies, PD-L2 antibodies, CTLA-4 antibodies, TIM3 antibodies, LAG3 antibodies, TIGIT antibodies); and cancer vaccines (i.e., anti-tumor vaccines or vaccines based on neoantigens such as a peptide or RNA vaccine).

Cell-based therapies (e.g., cancer vaccines), usually involve the removal of immune cells from a subject suffering from cancer, either from the blood or from a tumor. Immune cells specific for the tumor will be activated, grown, and returned to a subject suffering from cancer where the immune cells provide an immune response against the cancer. Cell types that can be used in this way are e.g., natural killer cells, lymphokine-activated killer cells, cytotoxic T-cells, dendritic cells, CAR-T therapies (i.e., chimeric antigen receptor T-cells which are T-cells engineered to target specific antigens), TIL therapy (i.e., administration of tumor-infiltrating lymphocytes), TCR gene therapy, protein vaccines, and nucleic acid vaccines. An exemplary cell-based therapy is Provenge. In some embodiments, the cell-based therapy is a CAR-T therapy.

Interleukin-2 and interferon-alpha are examples of cytokines, proteins that regulate and coordinate the behavior of the immune system.

Cancer Vaccines with Neoantigens

Neoantigens are antigens encoded by tumor-specific mutated genes. Technological innovations have made it possible to dissect the immune response to patient-specific neoantigens that arise as a consequence of tumor-specific mutations, and emerging data suggest that recognition of such neoantigens is a major factor in the activity of clinical immunotherapies. These observations indicate that neoantigen load may form a biomarker in cancer immunotherapy. Many novel therapeutic approaches are being developed that selectively enhance T cell reactivity against this class of antigens. One approach to target neoantigens is via cancer vaccine. These vaccines can be developed using peptides or RNA, e.g., synthetic peptides or synthetic RNA.

Antibody therapies are antibody proteins produced by the immune system and that bind to a target antigen on the surface of a cell. Antibodies are typically encoded by an immunoglobulin gene or genes, or fragments thereof. In normal physiology antibodies are used by the immune system to fight pathogens. Each antibody is specific to one or a few proteins, and those that bind to cancer antigens are used, e.g., for the treatment of cancer.

Antibodies are capable of specifically binding an antigen or epitope. (Fundamental Immunology, 3^{rd} Edition, W. E., Paul, ed., Raven Press, N.Y. (1993). Specific binding occurs to the corresponding antigen or epitope even in the presence of a heterogeneous population of proteins and other biologics. Specific binding of an antibody indicates that it binds to its target antigen or epitope with an affinity that is substantially greater than binding to irrelevant antigens. The relative difference in affinity is often at least 25% greater, more often at least 50% greater, most often at least 100% greater. The relative difference can be at least 2-fold, at least 5-fold, at least 10-fold, at least 25-fold, at least 50-fold, at least 100-fold, or at least 1000-fold, for example.

Exemplary types of antibodies include without limitation human, humanized, chimeric, monoclonal, polyclonal, single chain, antibody binding fragments, and diabodies. Once bound to a cancer antigen, antibodies can induce antibody-dependent cell-mediated cytotoxicity, activate the complement system, prevent a receptor interacting with its ligand or deliver a payload of chemotherapy or radiation, all of which can lead to cell death. Exemplary antibodies for the treatment of cancer include but are not limited to, Alemtuzumab, Bevacizumab, Bretuximab vedotin, Cetuximab, Gemtuzumab ozogamicin, Ibritumomab tiuxetan, Ipilimumab, Ofatumumab, Panitumumab, Rituximab, Tositumomab, Trastuzumab, Nivolumab, Pembrolizumab, Avelumab, durvalumab and pidilizumab.

### Checkpoint Blocking Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a compound (e.g., an inhibitor or antibody) that inhibits the immune checkpoint blockade pathway. Immune checkpoint proteins, under normal physiological conditions, maintain self-tolerance (e.g., prevent autoimmunity) and protect tissues from damage when the immune system is responding to e.g., pathogenic infection. Immune checkpoint proteins can be dysregulated by tumors as an important immune resistance mechanism. (Pardoll, Nature Rev. Cancer, 2012, 12, 252-264). Agonists of co-stimulatory receptors or antagonists of inhibitory signals (e.g., immune checkpoint proteins), provide an amplification of antigen-specific T-cell responses. Antibodies that block immune checkpoints do not target tumor cells directly but typically target lymphocyte receptors or their ligands to enhance endogenous antitumor activity.

Exemplary checkpoint blocking antibodies include but are not limited to, anti-CTLA-4, anti-PD-1, anti-LAG3 (i.e., antibodies against lymphocyte activation gene 3), and anti-TIM3 (i.e., antibodies against T-cell membrane protein 3). Exemplary anti-CTLA-4 antibodies include but are not limited to, ipilimumab and tremelimumab. Exemplary anti-PD-1 ligands include but are not limited to, PD-L1 (i.e., B7-H1 and CD274) and PD-L2 (i.e., B7-DC and CD273). Exemplary anti-PD-1 antibodies include but are not limited to, nivolumab (i.e., MDX-1106, BMS-936558, or ONO-4538)), CT-011, AMP-224, pembrolizumab (trade name Keytruda), and MK-3475. Exemplary PD-L1-specific antibodies include but are not limited to, BMS936559 (i.e., MDX-1105), MEDI4736 and MPDL-3280A. Exemplary checkpoint blocking antibodies also include but are not limited to, IMP321 and MGA271.

T-regulatory cells (e.g., CD4+, CD25+, or T-reg) are also involved in policing the distinction between self and non-self (e.g., foreign) antigens, and may represent an important mechanism in suppression of immune response in many cancers. T-reg cells can either emerge from the thymus (i.e., “natural T-reg”) or can differentiate from mature T-cells under circumstances of peripheral tolerance induction (i.e., “induced T-reg”). Strategies that minimize the action of T-reg cells would therefore be expected to facilitate the immune response to tumors. (Sutmuller, van Duivernvoorde et al., 2001).

### IDO Pathway Inhibitors

The IDO pathway regulates immune response by suppressing T cell function and enabling local tumor immune escape. IDO expression by antigen-presenting cells (APCs) can lead to tryptophan depletion, and resulting antigen-specific T cell energy and regulatory T cell recruitment. Some tumors even express IDO to shield themselves from the immune system. A compound that inhibits IDO or the IDO pathway thereby activating the immune system to attack the cancer (e.g., tumor in a subject). Exemplary IDO pathway inhibitors include indoximod, epacadostat and EOS200271.

### STING Pathway Agonists

Stimulator of interferon genes (STING) is an adaptor protein that plays an important role in the activation of type I interferons in response to cytosolic nucleic acid ligands. Evidence indicates involvement of the STING pathway in the induction of antitumor immune response. It has been shown that activation of the STING-dependent pathway in cancer cells can result in tumor infiltration with immune cells and modulation of the anticancer immune response. STING agonists are being developed as a class of cancer therapeutics. Exemplary STING agonists include MK-1454 and ADU-S100.

### Co-Stimulatory Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a co-stimulatory inhibitor or antibody. In some embodiments, the methods described herein comprise depleting or activating anti-4-1BB, anti-OX40, anti-GITR, anti-CD27 and anti-CD40, and variants thereof.

Inventive methods of the present invention contemplate single as well as multiple administrations of a therapeutically effective amount of a compound as described herein. Compounds, e.g., a compound as described herein, can be administered at regular intervals, depending on the nature, severity and extent of the subject's condition. In some embodiments, a compound described herein is administered in a single dose. In some embodiments, a compound described herein is administered in multiple doses.

### Inflammatory Disease

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat an inflammatory disease. As used herein, the term “inflammatory disease” refers to a disease or condition characterized by aberrant inflammation (e.g. an increased level of inflammation compared to a control such as a healthy person not suffering from a disease). Examples of inflammatory diseases include postoperative cognitive dysfunction, arthritis (e.g., rheumatoid arthritis, psoriatic arthritis, juvenile idiopathic arthritis), systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, Vitiligo, asthma (e.g., allergic asthma), acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis. Proteins associated with inflammation and inflammatory diseases (e.g. aberrant expression being a symptom or cause or marker of the disease) include interleukin-6 (IL-6), interleukin-8 (IL-8), interleukin-18 (IL-18), TNF-α (tumor necrosis factor-alpha), and C-reactive protein (CRP).

In some embodiments, the inflammatory disease comprises postoperative cognitive dysfunction, arthritis (e.g., rheumatoid arthritis, psoriatic arthritis, or juvenile idiopathic arthritis), systemic lupus erythematosus (SLE), myasthenia gravis, diabetes (e.g., juvenile onset diabetes or diabetes mellitus type 1), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, asthma (e.g., allergic asthma), acne vulgaris, celiac disease, chronic prostatitis, pelvic inflammatory disease, reperfusion injury, sarcoidosis, transplant rejection, interstitial cystitis, atherosclerosis, or atopic dermatitis.

In some embodiments, the inflammatory disease comprises postoperative cognitive dysfunction, which refers to a decline in cognitive function (e.g. memory or executive function (e.g. working memory, reasoning, task flexibility, speed of processing, or problem solving)) following surgery.

In other embodiments, the method of treatment is a method of prevention. For example, a method of treating postsurgical cognitive dysfunction may include preventing postsurgical cognitive dysfunction or a symptom of postsurgical cognitive dysfunction or reducing the severity of a symptom of postsurgical cognitive dysfunction by administering a compound described herein prior to surgery.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat an inflammatory disease (e.g., an inflammatory disease described herein) by decreasing or eliminating a symptom of the disease. In some embodiments, the compound of Formula (I), Formula (II), Formula (III-a) or Formula (II-b), or a pharmaceutically acceptable salt, solvate, hydrate, tautomer, ester, N-oxide or stereoisomer thereof may be used as a single agent in a composition or in combination with another agent in a composition to treat an inflammatory disease (e.g., an inflammatory disease described herein).

### Musculoskeletal Diseases

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a musculoskeletal disease. As used herein, the term “musculoskeletal disease” refers to a disease or condition in which the function of a subject's musculoskeletal system (e.g., muscles, ligaments, tendons, cartilage, or bones) becomes impaired. Exemplary musculoskeletal diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include muscular dystrophy (e.g., Duchenne muscular dystrophy, Becker muscular dystrophy, distal muscular dystrophy, congenital muscular dystrophy, Emery-Dreifuss muscular dystrophy, facioscapulohumeral muscular dystrophy, myotonic muscular dystrophy type 1, or myotonic muscular dystrophy type 2), limb girdle muscular dystrophy, multisystem proteinopathy, rhizomelic chondrodysplasia punctata, X-linked recessive chondrodysplasia punctata, Conradi-Hünermann syndrome, Autosomal dominant chondrodysplasia punctata, stress induced skeletal disorders (e.g., stress induced osteoporosis), multiple sclerosis, amyotrophic lateral sclerosis (ALS), primary lateral sclerosis, progressive muscular atrophy, progressive bulbar palsy, pseudobulbar palsy, spinal muscular atrophy, progressive spinobulbar muscular atrophy, spinal cord spasticity, spinal muscle atrophy, myasthenia gravis, neuralgia, fibromyalgia, Machado-Joseph disease, Paget's disease of bone, cramp fasciculation syndrome, Freidrich's ataxia, a muscle wasting disorder (e.g., muscle atrophy, sarcopenia, cachexia), an inclusion body myopathy, motor neuron disease, or paralysis.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a musculoskeletal disease (e.g., a musculoskeletal disease described herein) by decreasing or eliminating a symptom of the disease. In some embodiments, the method of treatment comprises treatment of muscle pain or muscle stiffness associated with a musculoskeletal disease. In some embodiments, the compound of Formula (I), Formula (II), Formula (III-a) or Formula (III-b), or a pharmaceutically acceptable salt, solvate, hydrate, tautomer, ester, N-oxide or stereoisomer thereof may be used as a single agent in a composition or in combination with another agent in a composition to treat a musculoskeletal disease (e.g., a musculoskeletal disease described herein).

### Metabolic Diseases

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat metabolic disease. As used herein, the term “metabolic disease” refers to a disease or condition affecting a metabolic process in a subject. Exemplary metabolic diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include non-alcoholic steatohepatitis (NASH), non-alcoholic fatty liver disease (NAFLD), liver fibrosis, obesity, heart disease, atherosclerosis, arthritis, cystinosis, diabetes (e.g., Type I diabetes, Type II diabetes, or gestational diabetes), phenylketonuria, proliferative retinopathy, or Kearns-Sayre disease.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a metabolic disease (e.g., a metabolic disease described herein) by decreasing or eliminating a symptom of the disease. In some embodiments, the method of treatment comprises decreasing or eliminating a symptom comprising elevated blood pressure, elevated blood sugar level, weight gain, fatigue, blurred vision, abdominal pain, flatulence, constipation, diarrhea, jaundice, and the like. In some embodiments, the compound of Formula (I), Formula (II), Formula (III-a) or Formula (III-b), or a pharmaceutically acceptable salt, solvate, hydrate, tautomer, or stereoisomer thereof may be used as a single agent in a composition or in combination with another agent in a composition to treat a metabolic disease (e.g., a musculoskeletal disease described herein).

### Mitochondrial Diseases

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat mitochondrial disease. As used herein, the term “mitochondrial disease” refers to a disease or condition affecting the mitochondria in a subject. In some embodiments, the mitochondrial disease is associated with, or is a result of, or is caused by mitochondrial dysfunction, one or more mitochondrial protein mutations, or one or more mitochondrial DNA mutations. In some embodiments, the mitochondrial disease is a mitochondrial myopathy. In some embodiments, mitochondrial diseases, e.g., the mitochondrial myopathy, that may be treated with a compound of Formula (I), Formula (II), Formula (III-a) or Formula (III-b), or a pharmaceutically acceptable salt, solvate, hydrate, tautomer, ester, N-oxide or stereoisomer thereof include, e.g., Barth syndrome, chronic progressive external ophthalmoplegia (cPEO), Kearns-Sayre syndrome (KSS), Leigh syndrome (e.g., MILS, or maternally inherited Leigh syndrome), mitochondrial DNA depletion syndromes (MDDS, e.g., Alpers syndrome), mitochondrial encephalomyopathy (e.g., mitochondrial encephalomyopathy, lactic acidosis, and stroke-like episodes (MELAS)), mitochondrial neurogastrointestinal encephalomyopathy (MNGIE), myoclonus epilepsy with ragged red fibers (MERRF), neuropathy, ataxia, retinitis pigmentosa (NARP), Leber's hereditary optic neuropathy (LHON), and Pearson syndrome.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a mitochondrial disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a mitochondrial disease described herein.

### Hearing Loss

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat hearing loss. As used herein, the term “hearing loss” or “hearing loss condition” may broadly encompass any damage to the auditory systems, organs, and cells or any impairment of an animal subject's ability to hear sound, as measured by standard methods and assessments known in the art, for example otoacoustic emission testing, pure tone testing, and auditory brainstem response testing. Exemplary hearing loss conditions that may be treated with a compound of Formula (I), Formula (II), Formula (III-a) or Formula (III-b), or a pharmaceutically acceptable salt, solvate, hydrate, tautomer, ester, N-oxide or stereoisomer thereof include, but are not limited to, mitochondrial nonsyndromic hearing loss and deafness, hair cell death, age-related hearing loss, noise-induced hearing loss, genetic or inherited hearing loss, hearing loss experienced as a result of ototoxic exposure, hearing loss resulting from disease, and hearing loss resulting from trauma. In some embodiments, mitochondrial nonsyndromic hearing loss and deafness is a MT-RNR1-related hearing loss. In some embodiments, the MT-RNR1-related hearing loss is the result of amino glycoside ototoxicity. In some embodiments, mitochondrial nonsyndromic hearing loss and deafness is a MT-TS1-related hearing loss. In some embodiments, mitochondrial nonsyndromic hearing loss and deafness is characterized by sensorineural hearing loss.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a hearing loss condition described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a hearing loss condition described herein.

### Ocular Disease

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat eye disease. As used herein, the term “ocular disease” may refer to a disease or condition in which the function of a subject's eye becomes impaired. Exemplary ocular diseases and conditions that may be treated with a compound of Formula (I), Formula (II), Formula (III-a) or Formula (III-b), or a pharmaceutically acceptable salt, solvate, hydrate, tautomer, ester, N-oxide or stereoisomer thereof include cataracts, glaucoma, endoplasmic reticulum (ER) stress, autophagy deficiency, age-related macular degeneration (AMD), or diabetic retinopathy.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat an ocular disease or condition described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat an ocular disease or condition described herein.

### Kidney Diseases

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat kidney disease. As used herein, the term “kidney disease” may refer to a disease or condition in which the function of a subject's kidneys becomes impaired. Exemplary kidney diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjögren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence. Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VGEF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome.

In some embodiments, the compound of Formula (I), Formula (II), Formula (III-a) or Formula (III-b), or a pharmaceutically acceptable salt, solvate, hydrate, tautomer, ester, N-oxide or stereoisomer thereof is used to treat a kidney disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a kidney disease described herein.

Skin Diseases

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a skin disease. As used herein, the term “skin disease” may refer to a disease or condition affecting the skin. Exemplary skin diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include acne, alopecia areata, basal cell carcinoma, Bowen's disease, congenital erythropoietic porphyria, contact dermatitis, Darier's disease, disseminated superficial actinic porokeratosis, dystrophic epidermolysis bullosa, eczema (atopic eczema), extra-mammary Paget's disease, epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, mucous membrane pemphigoid, pemphigoid, pemphigus vulgaris, pityriasis lichenoides, pityriasis rubra pilaris, plantar warts (verrucas), polymorphic light eruption, psoriasis, plaque psoriasis, pyoderma gangrenosum, rosacea, scabies, scleroderma, shingles, squamous cell carcinoma, sweet's syndrome, urticaria and angioedema and vitiligo.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a skin disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a skin disease described herein.

### Fibrotic Diseases

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a fibrotic disease. As used herein, the term “fibrotic disease” may refer to a disease or condition that is defined by the accumulation of excess extracellular matrix components. Exemplary fibrotic diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include adhesive capsulitis, arterial stiffness, arthrofibrosis, atrial fibrosis, cardiac fibrosis, cirrhosis, congenital hepatic fibrosis, Crohn's disease, cystic fibrosis, Dupuytren's contracture, endomyocardial fibrosis, glial scar, hepatitis C, hypertrophic cardiomyopathy, hypersensitivity pneumonitis, idiopathic pulmonary fibrosis, idiopathic interstitial pneumonia, interstitial lung disease, keloid, mediastinal fibrosis, myelofibrosis, nephrogenic systemic fibrosis, non-alcoholic fatty liver disease, old myocardial infarction, Peyronie's disease, pneumoconiosis, pneumonitis, progressive massive fibrosis, pulmonary fibrosis, radiation-induced lung injury, retroperitoneal fibrosis, scleroderma/systemic sclerosis, silicosis and ventricular remodeling.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a fibrotic disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a fibrotic disease described herein.

### Hemoglobin Disorders

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a hemoglobin disease. As used herein, the terms “hemoglobin disease” or “hemoglobin disorder” may refer to a disease or condition characterized by an abnormal production or structure of the hemoglobin protein. Exemplary hemoglobin diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include “dominant” β-thalassemia, acquired (toxic) methemoglobinemia, carboxyhemoglobinemia, congenital Heinz body hemolytic anemia, HbH disease, HbS/β-thalassemia, HbE/β-thalassemia, HbSC disease, homozygous α⁺-thalassemia (phenotype of α⁰-thalassemia), Hydrops fetalis with Hb Bart's, sickle cell anemia/disease, sickle cell trait, sickle β-thalassemia disease, α⁺-thalassemia, α⁰-thalassemia, α-Thalassemia associated with myelodysplastic syndromes, α-Thalassemia with mental retardation syndrome (ATR), β⁰-Thalassemia, β⁺-Thalassemia, δ-Thalassemia, γ-Thalassemia, β-Thalassemia major, β-Thalassemia intermedia, δβ-Thalassemia, and εγδβ-Thalassemia.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a hemoglobin disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a hemoglobin disease described herein.

### Autoimmune Diseases

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat an autoimmune disease. As used herein, the term “autoimmune disease” may refer to a disease or condition in which the immune system of a subject attacks and damages the tissues of said subject. Exemplary kidney diseases that may be treated with a compound of Formula (I), Formula (II), Formula (III-a) or Formula (III-b), or a pharmaceutically acceptable salt, solvate, hydrate, tautomer, ester, N-oxide or stereoisomer thereof include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat an autoimmune disease described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat an autoimmune disease described herein.

### Viral Infections

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a viral infection. Exemplary viral infections that may be treated with a compound of Formula (I), Formula (II), Formula (III-a) or Formula (III-b), or a pharmaceutically acceptable salt, solvate, hydrate, tautomer, ester, N-oxide or stereoisomer thereof include influenza, human immunodeficiency virus (HIV) and herpes.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a viral infection described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a viral infection described herein.

### Malaria Infection

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a malaria. As used herein, the term “malaria” may refer to a parasitic disease of protozoan of the plasmodium genus that causes infection of red blood cells (RBCs). Exemplary forms of malaria infection that may be treated with a compound of Formula (I), Formula (II), Formula (III-a) or Formula (III-b), or a pharmaceutically acceptable salt, solvate, hydrate, tautomer, ester, N-oxide or stereoisomer thereof include infection caused by Plasmodium vivax, Plasmodium ovale, Plasmodium malariae and Plasmodium falciparum. In some embodiments, the malaria infection that may be treated with a compound of Formula (I), Formula (II), Formula (III-a) or Formula (III-b), or a pharmaceutically acceptable salt, solvate, hydrate, tautomer, ester, N-oxide or stereoisomer thereof is resistant/recrudescent malaria.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a malaria infection described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a malaria infection described herein.

Diseases with Mutations Leading to Unfolded Protein Response (UPR) Induction

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a disease with mutations that leads to UPR induction. Exemplary disease with mutations that lead to UPR induction include Marinesco-Sjogren syndrome, neuropathic pain, diabetic neuropathic pain, noise induced hearing loss, non-syndromic sensorineural hearing loss, age-related hearing loss, Wolfram syndrome, Darier White disease, Usher syndrome, collagenopathies, Thin basement nephropathy, Alport syndrome, skeletal chondrodysplasia, metaphyseal chondrodysplasia type Schmid, and Pseudochondrodysplasia.

In some embodiments, the compound of Formula (I), Formula (II), Formula (III-a) or Formula (III-b) or a pharmaceutically acceptable salt, solvate, hydrate, tautomer, ester, N-oxide or stereoisomer thereof is used to treat a disease with mutations that leads to UPR induction described herein by decreasing or eliminating a symptom of the disease. In some embodiments, the compound of Formula (I), Formula (II), Formula (III-a) or Formula (III-b) or a pharmaceutically acceptable salt, solvate, hydrate, tautomer, ester, N-oxide or stereoisomer thereof may be used as a single agent in a composition or in combination with another agent in a composition to treat a disease with mutations that leads to UPR induction described herein.

### Methods of Modulating Protein Production

In another aspect, disclosed herein is a method of modulating the expression of eIF2B, eIF2α, a component of the eIF2 pathway, component of the ISR pathway or any combination thereof in a cell, the method comprising contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof, thereby modulating the expression of eIF2B, eIF2α, a component of the eIF2 pathway, component of the ISR pathway or any combination thereof in the cell. In some embodiments, contacting the compound of Formula (I), Formula (II), Formula (III-a) or Formula (III-b), or a pharmaceutically acceptable salt, solvate, hydrate, tautomer, ester, N-oxide or stereoisomer thereof with the cell increases the expression of eIF2B, eIF2α, a component of the eIF2 pathway, component of the ISR pathway or any combination thereof in the cell. In some embodiments, contacting the compound of Formula (I), Formula (II), Formula (III-a) or Formula (III-b), or a pharmaceutically acceptable salt, solvate, hydrate, tautomer, ester, N-oxide or stereoisomer thereof with the cell decreases the expression of eIF2B, eIF2α, a component of the eIF2 pathway, component of the ISR pathway or any combination thereof in the cell.

In another aspect, disclosed herein is a method of preventing or treating a condition, disease or disorder described herein in a patient in need thereof, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the compound of Formula (I), Formula (II), Formula (III-a) or Formula (III-b), or a pharmaceutically acceptable salt, solvate, hydrate, tautomer, ester, N-oxide or stereoisomer thereof modulates the expression of eIF2B, eIF2α, a component of the eIF2 pathway, component of the ISR pathway or any combination thereof by the patient's cells, thereby treating the condition, disease or disorder. In some embodiments, the condition, disease or disorder is characterized by aberrant expression of eIF2B, eIF2α, a component of the eIF2 pathway, component of the ISR pathway or any combination thereof by the patient's cells. In some embodiments, the compound of Formula (I), Formula (II), Formula (III-a) or Formula (III-b), or a pharmaceutically acceptable salt, solvate, hydrate, tautomer, ester, N-oxide or stereoisomer thereof increases the expression of eIF2B, eIF2α, a component of the eIF2 pathway, component of the ISR pathway or any combination thereof by the patient's cells, thereby treating the condition, disease or disorder. In some embodiments, the compound of Formula (I), Formula (II), Formula (III-a) or Formula (III-b), or a pharmaceutically acceptable salt, solvate, hydrate, tautomer, ester, N-oxide or stereoisomer thereof decreases the expression of eIF2B, eIF2α, a component of the eIF2 pathway, component of the ISR pathway or any combination thereof by the patient's cells, thereby treating the condition, disease or disorder.

In another aspect, disclosed herein is a method of modulating the activity of eIF2B, eIF2α, a component of the eIF2 pathway, component of the ISR pathway or any combination thereof in a cell, the method comprising contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof, thereby modulating the activity of eIF2B, eIF2α, a component of the eIF2 pathway, component of the ISR pathway or any combination thereof in the cell. In some embodiments, contacting the compound of Formula (I), Formula (II), Formula (III-a) or Formula (III-b), or a pharmaceutically acceptable salt, solvate, hydrate, tautomer, ester, N-oxide or stereoisomer thereof with the cell increases the activity of eIF2B, eIF2α, a component of the eIF2 pathway, component of the ISR pathway or any combination thereof in the cell. In some embodiments, contacting the compound of Formula (I), Formula (II), Formula (III-a) or Formula (III-b), or a pharmaceutically acceptable salt, solvate, hydrate, tautomer, ester, N-oxide or stereoisomer thereof with the cell decreases the activity of eIF2B, eIF2α, a component of the eIF2 pathway, component of the ISR pathway or any combination thereof in the cell.

In another aspect, disclosed herein is a method of preventing or treating a condition, disease or disorder described herein in a patient in need thereof, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the compound of Formula (I), Formula (II), Formula (III-a) or Formula (III-b), or a pharmaceutically acceptable salt, solvate, hydrate, tautomer, ester, N-oxide or stereoisomer thereof modulates the activity of eIF2B, eIF2α, a component of the eIF2 pathway, component of the ISR pathway or any combination thereof by the patients cells, thereby treating the condition, disease or disorder. In some embodiments, the condition, disease or disorder is characterized by aberrant activity of eIF2B, eIF2α, a component of the eIF2 pathway, component of the ISR pathway or any combination thereof in the patient's cells. In some embodiments, the compound of Formula (I), Formula (II), Formula (III-a) or Formula (III-b), or a pharmaceutically acceptable salt, solvate, hydrate, tautomer, ester, N-oxide or stereoisomer thereof increases the activity of eIF2B, eIF2α, a component of the eIF2 pathway, component of the ISR pathway or any combination thereof in the patient's cells, thereby treating the condition, disease or disorder. In some embodiments, the compound of Formula (I), Formula (II), Formula (III-a) or Formula (III-b), or a pharmaceutically acceptable salt, solvate, hydrate, tautomer, ester, N-oxide or stereoisomer thereof decreases the activity of eIF2B, eIF2α, a component of the eIF2 pathway, component of the ISR pathway or any combination thereof in the patient's cells, thereby treating the condition, disease or disorder.

In some embodiments, administering an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the compound of Formula (I), Formula (II), Formula (III-a) or Formula (III-b), or a pharmaceutically acceptable salt, solvate, hydrate, tautomer, ester, N-oxide or stereoisomer thereof modulates both the expression and the activity of eIF2B, eIF2α, a component of the eIF2 pathway, component of the ISR pathway or any combination thereof in the patients cells, thereby treating the condition, disease or disorder.

In some embodiments, the compound of Formula (I), Formula (II), Formula (III-a) or Formula (III-b) is chemically modified, prior to (ex vivo) or after (in vivo) contacting with a cell, forming a biologically active compound that modulates the expression and/or activity of eIF2B, eIF2α, a component of the eIF2 pathway, component of the ISR pathway or any combination thereof in the cell. In some embodiments, the compound of Formula (I), Formula (II), Formula (III-a) or Formula (III-b) is metabolized by the patient forming a biologically active compound that modulates the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of formula (II).

In one aspect, disclosed herein is a method of treating a disease related to a modulation of eIF2B activity or levels, eIF2α activity or levels, or the activity or levels of a component of the eIF2 pathway or the ISR pathway in a patient in need thereof, comprising administering to the patient an effective amount of a compound of Formula (I), Formula (II), Formula (III-a) or Formula (III-b). In some embodiments, the modulation comprises an increase in eIF2B activity or levels, increase in eIF2α activity or levels, or increase in activity or levels of a component of the eIF2 pathway or the ISR pathway. In some embodiments, the disease may be caused by a mutation to a gene or protein sequence related to a member of the eIF2 pathway (e.g., the eIF2α signaling pathway).

### Methods of Increasing Protein Activity and Production

In another aspect, the compound of Formula (I), Formula (II), Formula (III-a) or Formula (III-b), or a pharmaceutically acceptable salt, solvate, hydrate, tautomer, ester, N-oxide or stereoisomer thereof may be useful in applications where increasing production output of eIF2B, eIF2α, a component of the eIF2 pathway, a component of the ISR pathway or any combination thereof is desirable, such as in vitro cell free systems for protein production.

In some embodiments, the present invention features a method of increasing expression of eIF2B, eIF2α, a component of the eIF2 pathway, a component of the ISR pathway or any combination thereof by a cell or in vitro expression system, the method comprising contacting the cell or in vitro expression system with an effective amount of a compound of Formula (I), Formula (II), Formula (III-a) or Formula (III-b), or a pharmaceutically acceptable salt, solvate, hydrate, tautomer, ester, N-oxide or stereoisomer thereof. In some embodiments, the method is a method of increasing the expression of eIF2B, eIF2α, a component of the eIF2 pathway, a component of the ISR pathway or any combination thereof by a cell comprising contacting the cell with an effective amount of a compound described herein (e.g., the compound of Formula (I), Formula (II), Formula (III-a) or Formula (III-b), or a pharmaceutically acceptable salt, solvate, hydrate, tautomer, ester, N-oxide or stereoisomer thereof). In other embodiments, the method is a method of increasing the expression of eIF2B, eIF2α a component of the eIF2 pathway, a component of the ISR pathway or any combination thereof by an in vitro protein expression system comprising contacting the in vitro expression system with a compound described herein (e.g. the compound of Formula (I), Formula (II), Formula (III-a) or Formula (III-b), or a pharmaceutically acceptable salt, solvate, hydrate, tautomer, ester, N-oxide or stereoisomer thereof). In some embodiments, contacting the cell or in vitro expression system with an effective amount of a compound of Formula (I), Formula (II), Formula (III-a) or Formula (III-b), or a pharmaceutically acceptable salt, solvate, hydrate, tautomer, ester, N-oxide or stereoisomer thereof increases expression of eIF2B, eIF2, a component of the eIF2 pathway, a component of the ISR pathway or any combination thereof in the cell or in vitro expression system by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%. In some embodiments, contacting the cell or in vitro expression system with an effective amount of a compound of Formula (I), Formula (II), Formula (III-a) or Formula (III-b), or a pharmaceutically acceptable salt, solvate, hydrate, tautomer, ester, N-oxide or stereoisomer thereof increases expression of eIF2B, eIF2α, a component of the eIF2 pathway, a component of the ISR pathway or any combination thereof in the cell or in vitro expression system by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold.

In some embodiments, the present invention features a method of increasing the expression of eIF2B, eIF2α, a component of the eIF2 pathway, a component of the ISR pathway or any combination thereof by a patient cells, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the patient has been diagnosed with a disease, disorder, or condition disclosed herein and wherein the disease, disorder or condition is characterized by aberrant expression of eIF2B, eIF2α, a component of the eIF2 pathway, a component of the ISR pathway or any combination thereof (e.g., a leukodystrophy, a leukoencephalopathy, a hypomyelinating or demyelinating disease, muscle-wasting disease, or sarcopenia). In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof increases the expression of eIF2B, eIF2a, a component of the eIF2 pathway, a component of the ISR pathway or any combination thereof by the patients cells about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition. In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof increases expression of eIF2B, eIF2α, a component of the eIF2 pathway, a component of the ISR pathway or any combination thereof by the patients cells about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold, thereby treating the disease, disorder or condition.

In another aspect, the compound of Formula (I), Formula (II), Formula (III-a) or Formula (III-b), or a pharmaceutically acceptable salt, solvate, hydrate, tautomer, ester, N-oxide or stereoisomer thereof may be useful in applications where increasing the activity of eIF2B, eIF2α, a component of the eIF2 pathway, a component of the ISR pathway or any combination thereof is desirable.

In some embodiments, the present invention features a method of increasing the activity of eIF2B, eIF2α, a component of the eIF2 pathway, a component of the ISR pathway or any combination thereof in a cell, the method comprising contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof. In some embodiments, contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%. In some embodiments, contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the cell by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold.

In some embodiments, the present invention features a method of increasing the activity of eIF2B, eIF2α, a component of the eIF2 pathway, a component of the ISR pathway or any combination thereof in a patient in need thereof, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the patient has been diagnosed with a disease, disorder, or condition disclosed herein and wherein the disease, disorder or condition is characterized by lowered levels of protein activity. In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition. In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the patient by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold, thereby treating the disease, disorder or condition.

In some embodiments, the compound of Formula (I), Formula (II), Formula (III-a) or Formula (III-b) is chemically modified, prior to (ex vivo) or after (in vivo) contacting with the cell or in vitro expression system, forming a biologically active compound that increases the expression and/or activity of eIF2B, eIF2α, a component of the eIF2 pathway, component of the ISR pathway or any combination thereof in the cells and/or in vitro expression system. In some embodiments, the compound of Formula (I), Formula (II), Formula (III-a) or Formula (III-b) is metabolized by the patient forming a biologically active compound that increases the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of formula (II).

### Methods of Decreasing Protein Activity and Production

In another aspect, the compound of Formula (I), Formula (II), Formula (III-a) or Formula (III-b), or a pharmaceutically acceptable salt, solvate, hydrate, tautomer, ester, N-oxide or stereoisomer thereof may be useful in applications where decreasing production output of eIF2B, eIF2α, a component of the eIF2 pathway, a component of the ISR pathway or any combination thereof is desirable.

In some embodiments, the present invention features a method of decreasing expression of eIF2B, eIF2α, a component of the eIF2 pathway, a component of the ISR pathway or any combination thereof in a cell, the method comprising contacting the cells with an effective amount of a compound of Formula (I), Formula (II), Formula (III-a) or Formula (III-b), or a pharmaceutically acceptable salt, solvate, hydrate, tautomer, ester, N-oxide or stereoisomer thereof. In some embodiments, contacting the cells with an effective amount of a compound of Formula (I), Formula (II), Formula (III-a) or Formula (III-b), or a pharmaceutically acceptable salt, solvate, hydrate, tautomer, ester, N-oxide or stereoisomer thereof decreases expression of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%.

In some embodiments, the present invention features a method of decreasing the expression of eIF2B, eIF2α, a component of the eIF2 pathway, a component of the ISR pathway or any combination thereof in a patient in need thereof, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the patient has been diagnosed with a disease, disorder, or condition described herein and wherein the disease, disorder or condition is characterized by increased levels of protein production. In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof decreases the expression of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In another aspect, the compound of Formula (I), Formula (II), Formula (III-a) or Formula (III-b), or a pharmaceutically acceptable salt, solvate, hydrate, tautomer, ester, N-oxide or stereoisomer thereof may be useful in applications where decreasing the activity of eIF2B, eIF2α, a component of the eIF2 pathway, a component of the ISR pathway or any combination thereof is desirable.

In some embodiments, the present invention features a method of decreasing the activity of eIF2B, eIF2α, a component of the eIF2 pathway, a component of the ISR pathway or any combination thereof in a cell, the method comprising contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof. In some embodiments, contacting the cell with an effective amount of a compound of Formula (I), Formula (II), Formula (III-a) or Formula (III-b), or a pharmaceutically acceptable salt, solvate, hydrate, tautomer, ester, N-oxide or stereoisomer thereof decreases the activity of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In some embodiments, the present invention features a method of decreasing the activity of eIF2B, eIF2α, a component of the eIF2 pathway, a component of the ISR pathway or any combination thereof in a patient in need thereof, the method comprising administering to the patient an effective amount of a compound of Formula (I), Formula (II), Formula (III-a) or Formula (III-b), or a pharmaceutically acceptable salt, solvate, hydrate, tautomer, ester, N-oxide or stereoisomer thereof, wherein the patient has been diagnosed with a disease, disorder, or condition described herein and wherein the disease, disorder or condition is characterized by increased levels of protein activity. In some embodiments, administering to the patient in need thereof an effective amount of a compound of Formula (I), Formula (II), Formula (III-a) or Formula (III-b), or a pharmaceutically acceptable salt, solvate, hydrate, tautomer, ester, N-oxide or stereoisomer thereof decreases the activity of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In some embodiments, the compound of Formula (I), Formula (II), Formula (III-a) or Formula (III-b) is chemically modified, prior to (ex vivo) or after (in vivo) contacting with a cell, forming a biologically active compound that decreases the expression and/or activity of eIF2B, eIF2α, a component of the eIF2 pathway, component of the ISR pathway or any combination thereof in the cell. In some embodiments, the compound of Formula (I), Formula (II), Formula (III-a) or Formula (III-b) is metabolized by the patient forming a biologically active compound that decreases the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of Formula (I), Formula (II), Formula (III-a) or Formula (III-b).

In some embodiments, the compounds set forth herein are provided as pharmaceutical compositions including a compound of Formula (I), Formula (II), Formula (III-a) or Formula (III-b) or a pharmaceutically acceptable salt, solvate, hydrate, tautomer, or stereoisomer thereof and a pharmaceutically acceptable excipient. In embodiments of the method, a compound of Formula (I), Formula (II), Formula (III-a) or Formula (III-b) or a pharmaceutically acceptable salt, solvate, hydrate, tautomer, or stereoisomer thereof, is co-administered with a second agent (e.g. therapeutic agent). In other embodiments of the method, a compound of Formula (I), Formula (II), Formula (III-a) or Formula (III-b) or a pharmaceutically acceptable salt, solvate, hydrate, tautomer, or stereoisomer thereof, is co-administered with a second agent (e.g. therapeutic agent), which is administered in a therapeutically effective amount. In embodiments, the second agent is an agent for improving memory.

### Combination Therapy

In one aspect, the present invention features a pharmaceutical composition comprising a compound of Formula (I), Formula (II), Formula (III-a) or Formula (III-b) or a pharmaceutically acceptable salt, solvate, hydrate, tautomer, or stereoisomer thereof as well as a second agent (e.g. a second therapeutic agent). In some embodiments, the pharmaceutical composition includes a second agent (e.g. a second therapeutic agent) in a therapeutically effective amount. In some embodiments, the second agent is an agent for treating cancer, a neurodegenerative disease, a leukodystrophy, an inflammatory disease, a musculoskeletal disease, a metabolic disease, or a disease or disorder associated with impaired function of eIF2B, eIF2α, or a component of the eIF2 pathway or ISR pathway.

The compounds described herein can be used in combination with one another, with other active agents known to be useful in treating cancer, a neurodegenerative disease, an inflammatory disease, a musculoskeletal disease, a metabolic disease, or a disease or disorder associated with impaired function of eIF2B, eIF2α, or a component of the eIF2 pathway or ISR pathway or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for a cancer, a neurodegenerative disease, a leukodystrophy, an inflammatory disease, a musculoskeletal disease, a metabolic disease, or a disease or disorder associated with impaired function of eIF2B, eIF2α, or a component of the eIF2 pathway or ISR pathway.

In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating a leukodystrophy. In embodiments, the second agent is an agent for treating vanishing white matter disease. In embodiments, the second agent is an agent for treating childhood ataxia with CNS hypo-myelination. In embodiments, the second agent is an agent for treating an intellectual disability syndrome. In embodiments, the second agent is an agent for treating pancreatic cancer. In embodiments, the second agent is an agent for treating breast cancer. In embodiments, the second agent is an agent for treating multiple myeloma. In embodiments, the second agent is an agent for treating myeloma. In embodiments, the second agent is an agent for treating a cancer of a secretory cell. In embodiments, the second agent is an agent for reducing eIF2α phosphorylation. In embodiments, the second agent is an agent for inhibiting a pathway activated by eIF2α phosphorylation. In embodiments, the second agent is an agent for inhibiting a pathway activated by eIF2α. In embodiments, the second agent is an agent for inhibiting the integrated stress response. In embodiments, the second agent is an anti-inflammatory agent. In embodiments, the second agent is an agent for treating postsurgical cognitive dysfunction. In embodiments, the second agent is an agent for treating traumatic brain injury. In embodiments, the second agent is an agent for treating a musculoskeletal disease. In embodiments, the second agent is an agent for treating a metabolic disease. In embodiments, the second agent is an anti-diabetic agent.

### Anti-Cancer Agents

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anticancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 1 1-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R^{II} retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol, i.e. paclitaxel), Taxotere, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and SC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-1 12378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A 1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tularik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-25041 1 (Sanofi), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ^{U 1}ln, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HK1-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ^{m}In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ^{m}Ag, ^{m}In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

### Additional Agents

In some embodiments, the second agent for use in combination with a compound (e.g., a compound of Formula (I), Formula (II), Formula (III-a) or Formula (III-b)) or composition thereof described herein is an agent for use in treating a neurodegenerative disease, a leukodystrophy, an inflammatory disease, a musculoskeletal disease, or a metabolic disease. In some embodiments, a second agent for use in combination with a compound (e.g., a compound of Formula (I), Formula (II), Formula (III-a) or Formula (III-b)) or composition thereof described herein is an agent approved by the FDA or similar regulatory agency of a country other than the USA, for treating a disease, disorder, or condition described herein.

In some embodiments, a second agent for use in treating a neurodegenerative disease, a leukodystrophy, an inflammatory disease, a musculoskeletal disease, or a metabolic disease includes, but is not limited to, an anti-psychotic drug, anti-depressive drug, anti-anxiety drug, analgesic, a stimulant, a sedative, a pain reliever, an anti-inflammatory agent, a benzodiazepine, a cholinesterase inhibitor, a non-steroidal anti-inflammatory drug (NSAID), a corticosteroid, a MAO inhibitor, a beta-blocker, a calcium channel blocker, an antacid, or other agent. Exemplary second agents may include donepezil, galantamine, rivastigmine, memantine, levodopa, dopamine, pramipexole, ropinirole, rotigotine, doxapram, oxazepam, quetiapine, selegiline, rasagiline, entacapone, benztropine, trihexyphenidyl, riluzole, diazepam, chlorodiazepoxide, lorazepam, alprazolam, buspirone, gepirone, ispapirone, hydroxyzine, propranolol, hydroxyzine, midazolam, trifluoperazine, methylphenidate, atomoxetine, methylphenidate, pemoline, perphenazine, divalproex, valproic acid, sertraline, fluoxetine, citalopram, escitalopram, paroxetine, fluvoxamine, trazodone, desvenlafaxine, duloxetine, venlafaxine, amitriptyline, amoxapine, clomipramine, desipramine, imipramine, nortriptyline, protriptyline, trimipramine, maprotiline, bupropion, nefazodone, vortioxetine, lithium, clozapine, fluphenazine, haloperidol, paliperidone, loxapine, thiothixene, pimozide, thioridazine, risperidone, aspirin, ibuprofen, naproxen, acetaminophen, azathioprine, methotrexate, mycophenolic acid, leflunomide, dibenzoylmethane, cilostazol, pentoxifylline, duloxetine, a cannabinoid (e.g, nabilone), simethicone, magaldrate, aluminum salts, calcium salts, sodium salts, magnesium salts, alginic acid, acarbose, albiglutide, alogliptin, metformin, insulin, lisinopril, atenolol, atorvastatin, fluvastatin, lovastatin, pitavastatin, simvastatin, rosuvastatin, and the like.

Naturally derived agents or supplements may also be used in conjunction with a compound of Formula (I), Formula (II), Formula (III-a) or Formula (III-b) or a composition thereof to treat a neurodegenerative disease, an inflammatory disease, a musculoskeletal disease, or a metabolic disease. Exemplary naturally derived agents or supplements include omega-3 fatty acids, carnitine, citicoline, curcumin, gingko, vitamin E, vitamin B (e.g., vitamin B5, vitamin B6, or vitamin B12), huperzine A, phosphatidylserine, rosemary, caffeine, melatonin, chamomile, St. John's wort, tryptophan, and the like.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General scheme relating to methods of making exemplary compounds of the invention are additionally described in the section entitled Methods of Making Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization; BTMG for 2-tert-butyl-1,1,3,3-tetramethylguanidine; DBU for 1,8-diazabicyclo[5.4.0]undec-7-ene; DCI for desorption chemical ionization; DIPEA for N,N-diisopropylethylamine; DMSO for dimethyl sulfoxide; ESI for electrospray ionization; HATU for 1-[bis(dimethylamino)methylene]-1H-1,2,3-triazolo[4,5-b]pyridinium 3-oxid hexafluorophosphate; HPLC for high performance liquid chromatography; LED for light-emitting diode; MS for mass spectrum; NMR for nuclear magnetic resonance; psi for pounds per square inch; SCX for strong cation exchange; SFC for supercritical fluid chromatography; T3P for 1-propanephosphonic anhydride; and TLC for thin-layer chromatography.

### Example 1: (2R)-6-chloro-N-(3-{5-[(3,5-dimethylphenoxy)methyl]-2-oxo-1,3-oxazolidin-3-yl}bicyclo[1.1.1]pentan-1-yl)-4-oxo-3,4-dihydro-2H-1-benzopyran-2-carboxamide (Compound 100)

### Example 1A: Tert-Butyl (3-(5-((3,5-dimethylphenoxy)methyl)-2-oxooxazolidin-3-yl)bicyclo[1.1.1]pentan-1-yl)carbamate

A 30 mL vial was charged with iodomesitylene diacetate (127 mg, 0.35 mmol), 3-((tert-butoxycarbonyl)amino)bicyclo[1.1.1]pentane-1-carboxylic acid (Enamine, 157 mg, 0.691 mmol) and toluene (5 mL), and the mixture was stirred at 55° C. for 30 minutes. Toluene was then removed under high vacuum. Iridium(III) bis[2-(2,4-difluorophenyl)-5-methylpyridine-N,C₂₀]-4,40-di-tert-butyl-2,20-bipyridine hexafluorophosphate (14 mg, 0.014 mmol), copper(I) thiophene-2-carboxylate (31.6 mg, 0.166 mmol), 4,7-diphenyl-1,10-phenanthroline (83 mg, 0.249 mmol), 2-tert-butyl-1,1,3,3-tetramethylguanidine (BTMG, 0.29 mL, 1.45 mmol) and metaxalone (153 mg, 0.691 mmol) were added sequentially followed by dioxane (5.0 mL). The vial was degassed by sparging with nitrogen for 3 minutes before sealing with a polytetrafluoroethylene-lined cap. The vial was then put inside a 250 mL glass Dewar filled with water and clamped at a 45 angle to increase exposure to the light-emitting diode (LED). (The glass Dewar was used to focus the blue LED to the vial, and the water bath was used to keep a constant temperature). The reaction was stirred and irradiated using 40W Kessil® PR160 390 nm Photoredox lamp just 5 cm above the vial. The bath temperature was measured as 22° C. when setting up the reaction and rose to 38° C. after an hour, and the temperature was stabilized at 38° C. for the remainder of the reaction time. After 48 hours, the reaction mixture was quenched by exposing to air and partitioned between water (50 mL) and dichloromethane (2×50 mL). The organic layers were combined and dried over sodium sulfate and concentrated under reduced pressure. The residue was taken up in methanol (5 mL), filtered through a glass microfiber frit and purified by preparative HPLC [YMC TriArt™ C18 Hybrid 5 μm column, 50×100 mm, flow rate 140 mL/minute, 2-100% gradient of acetonitrile in buffer (0.025 M aqueous ammonium bicarbonate, adjusted to pH 10 with ammonium hydroxide)] to give the title compound (12.6 mg, 0.031 mmol, 4.5% yield). ¹H NMR (400 MHz, methanol-d₄) δ ppm 6.59 (s, 1H), 6.53 (s, 2H), 4.69-4.44 (m, 1H), 4.16-4.01 (m, 2H), 3.75 (t, J=9.0 Hz, 1H), 3.53 (dd, J=8.8, 6.1 Hz, 1H), 2.29 (s, 6H), 2.24 (s, 6H), 1.42 (s, 9H); MS (APCI⁺) m/z 403 (M+H)⁺.

### Example 1B: (R)-6-chloro-4-oxochroman-2-carboxylic Acid

6-Chloro-4-oxo-3,4-dihydro-2H-1-benzopyran-2-carboxylic acid (Princeton) was purified by preparative chiral supercritical fluid chromatography (SFC) [performed on a Thar 200 preparative SFC (SFC-5) system using a Daicel CHIRALPAK® AD-H, 30×250 mm I.D., 5 μm column. The column was heated at 38° C., and the backpressure regulator was set to maintain 100 bar. The mobile phase was 40% methanol in carbon dioxide at a flow rate of 80 g/minute] to give the title compound as the earlier eluting fraction. MS (ESI⁻) m/z 225 (M−H)⁻.

### Example 1C: (2R)-6-chloro-N-(3-{S-[(3,5-dimethylphenoxy)methyl]-2-oxo-1,3-oxazolidin-3-yl}bicyclo[1.1.1]pentan-1-yl)-4-oxo-3,4-dihydro-2H-1-benzopyran-2-carboxamide

The product of Example 1A (7 mg, 0.031 mmol) was combined with trifluoroacetic acid (0.1 mL) and stirred at ambient temperature for 30 minutes, and then the mixture was concentrated under reduced pressure. The product of Example 1B (7 mg, 0.031 mmol), triethylamine (0.017 mL), N,N-dimethylformamide (1.0 mL) and 1-[bis(dimethylamino)methylene]-1H-1,2,3-triazolo[4,5-b]pyridinium 3-oxid hexafluorophosphate (HATU, 15.3 mg, 0.04 mmol) were added sequentially, and the resulting reaction mixture was stirred at ambient temperature for 3 hours. Water (0.1 mL) was added and the resulting solution was filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (13 mg, 0.025 mmol, 82% yield). ¹H NMR (400 MHz, DMSO-d₆) δ ppm 8.65 (s, 1H), 7.65 (d, J=2.7 Hz, 1H), 7.58 (dd, J=8.8, 2.7 Hz, 1H), 7.14 (d, J=8.7 Hz, 1H), 6.60 (s, 1H), 6.56 (s, 2H), 5.05 (t, J=7.1 Hz, 1H), 4.83-4.72 (m, 1H), 4.09 (qd, J=11.1, 4.4 Hz, 2H), 3.68 (t, J=8.8 Hz, 1H), 3.39 (dd, J=8.8, 6.3 Hz, 1H), 2.94 (d, J=7.1 Hz, 2H), 2.29 (s, 6H), 2.23 (s, 6H); MS (APCI⁺) m/z 511 (M+H)⁺.

### Example 2: (2R)-6-chloro-N-(1R,3r,5S)-8-[3-(4-chlorophenoxy)propyl]-8-azabicyclo[3.2.1]octan-3-yl)-4-oxo-3,4-dihydro-2H-1-benzopyran-2-carboxamide (Compound 101)

### Example 2A: (1R,3r,5S)-8-(3-(4-chlorophenoxy)propy)-8-azabicyclo[3.2.1]octan-3-amine

rac-tert-Butyl ((1R,5S)-8-azabicyclo[3.2.1]octan-3-yl)carbamate (Combi-Blocks, 155 mg, 0.685 mmol), 1-(3-bromopropoxy)-4-chlorobenzene (Enamine, 188 mg, 0.75 mmol) and N,N-diisopropylethylamine (0.5 mL) were combined with dimethyl sulfoxide (1 mL) and stirred at 90° C. for 18 hours. The reaction mixture was cooled to ambient temperature and partitioned between water (50 mL) and dichloromethane (2×30 mL). The organic phases were combined, dried over sodium sulfate, and concentrated under reduced pressure. The residue was taken up in dichloromethane (2 mL) and trifluoroacetic acid (2 mL) was added. After stirring at ambient temperature for 1 hour, the solution was concentrated under reduced pressure, and the residue was purified by preparative HPLC [Waters XBridge™ C18 5 μm OBD column, 50×100 mm, flow rate 90 mL/minute, 5-100% gradient of acetonitrile in buffer (0.025 M aqueous ammonium bicarbonate, adjusted to pH 10 with ammonium hydroxide)] to give the title compound (0.13 g, 0.44 mmol, 64% yield). ¹H NMR (400 MHz, DMSO-d₆-D₂O) δ ppm 7.32-7.26 (m, 2H), 6.96-6.90 (m, 2H), 3.98 (t, J=6.3 Hz, 2H), 3.09-2.99 (m, 3H), 2.37 (t, J=7.3 Hz, 2H), 1.98-1.85 (m, 4H), 1.84-1.73 (m, 4H), 1.36-1.23 (m, 2H); MS (APCI⁺) m/z 295 (M+H)⁺.

### Example 2B: (2R)-6-chloro-N-{(1R,3r,5S)-8-[3-(4-chlorophenoxy)propyl]-8-azabicyclo[3.2.1]octan-3-yl}-4-oxo-3,4-dihydro-2H-1-benzopyran-2-carboxamide

The product of Example 1B (15 mg, 0.068 mmol), the product of Example 2A (20 mg, 0.068 mmol) and triethylamine (0.019 mL) were combined with N,N-dimethylformamide (1 mL) and stirred at ambient temperature. 1-[Bis(dimethylamino)methylene]-1H-1,2,3-triazolo[4,5-b]pyridinium 3-oxid hexafluorophosphate (HATU, 28 mg, 0.075 mmol) was added in one portion. After stirring at ambient temperature for 30 minutes, water (0.2 mL) was added to the reaction mixture. The resulting solution was filtered through a glass microfiber frit and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (25 mg, 0.050 mmol, 73% yield). ¹H NMR (400 MHz, DMSO-d₆) δ ppm 7.78 (d, J=5.5 Hz, 1H), 7.68-7.59 (m, 2H), 7.34-7.26 (m, 2H), 7.17 (d, J=8.6 Hz, 1H), 6.98-6.90 (m, 2H), 5.21 (dd, J=7.5, 5.1 Hz, 1H), 4.01 (t, J=6.4 Hz, 2H), 3.77-3.71 (m, 1H), 3.11-2.91 (m, 4H), 2.36 (t, J=7.0 Hz, 2H), 2.00-1.69 (m, 7H), 1.62-1.43 (m, 3H); MS (APCI⁺) m/z 503 (M+H)⁺.

### Example 3: (2R,4R)-6-chloro-N-[(1r,4R)-4-{[(4-chloro-3-fluorophenoxy)acetyl](methyl)amino}cyclohexyl]-4-hydroxy-3,4-dihydro-2H-1-benzopyran-2-carboxamide (Compound 102)

### Example 3A: Tert-Butyl ((1r,4r)-4-(2-(4-chloro-3-fluorophenoxy)-N-methylacetamido)cyclohexyl)carbamate

The reaction and purification conditions described in Example 2B substituting 2-(4-chloro-3-fluorophenoxy)acetic acid for the product of Example 1B, and tert-butyl (trans-4-(methylamino)cyclohexyl)carbamate for the product of Example 2A gave the title compound. MS (APCI⁺) m/z 415 (M+H)⁺.

### Example 3B: (2R,4RV-6-chloro-4-hydroxychroman-2-carboxylic Acid

The product of Example 1B (250 mg, 1.1 mmol) was dissolved in methanol (2 mL) and stirred at ambient temperature. Sodium borohydride (167 mg, 4.41 mmol) was added. After stirring for 5 minutes, saturated ammonium chloride solution (1 mL) was added. After stirring for another 10 minutes, the resulting mixture was combined with diatomaceous earth (10 g) and concentrated under reduced pressure to give a free flowing powder. The powder was directly purified by reversed-phase flash chromatography [Interchim PuriFlash C18XS 30 μm 175 g column, flow rate 100 mL/minute, 5-100% gradient of acetonitrile in buffer (0.1% trifluoroacetic acid)] to give the title compound (0.24 g, 1.05 mmol, 95% yield). MS (APCI⁻) m/z 227 (M−H)⁻.

### Example 3C: (2R,4R)-6-chloro-N-[(1r,4R)-4-{[(4-chloro-3-fluorophenoxy)acetyl](methyl)amino}cyclohexyl]-4-hydroxy-3,4-dihydro-2H-1-benzopyran-2-carboxamide

The product of Example 3A (34 mg, 0.082 mmol) and trifluoroacetic acid (0.5 mL) were combined and stirred at 25° C. for 30 minutes and then concentrated under reduced pressure. To the residue was added N,N-dimethylformamide (2 mL), the product of Example 3B (20.6 mg, 0.090 mmol) and N,N-diisopropylethylamine (0.114 mL). While stirring, 1-propanephosphonic anhydride (T3P, 50 weight % solution in N,N-dimethylformamide, 0.057 mL) was added drop-wise over 2 minutes, and the resulting mixture was stirred for 1 hour and then partitioned between dichloromethane (2×25 mL) and aqueous sodium carbonate (1.0 M, 20 mL). The organic layers were combined, dried over anhydrous sodium sulfate, and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (26 mg, 0.049 mmol, 60% yield). ¹H NMR (120° C., 400 MHz, DMSO-d₆) δ ppm 7.45-7.37 (m, 2H), 7.34 (d, J=8.1 Hz, 1H), 7.15 (dd, J=8.8, 2.7 Hz, 1H), 6.97 (dd, J=11.4, 2.8 Hz, 1H), 6.87 (d, J=8.7 Hz, 1H), 6.82 (ddd, J=8.9, 2.8, 1.3 Hz, 1H), 5.23 (d, J=5.9 Hz, 1H), 4.87-4.77 (m, 3H), 4.60 (dd, J=11.3, 2.8 Hz, 1H), 3.96 (br s, 1H), 3.70-3.57 (m, 1H), 2.84 (s, 3H), 2.42 (ddd, J=13.2, 5.9, 2.9 Hz, 1H), 1.98-1.88 (m, 2H), 1.83 (dt, J=13.1, 10.5 Hz, 1H), 1.74-1.61 (m, 4H), 1.54-1.37 (m, 2H); MS (APCI⁺) m/z 507 (M−H₂O+H)⁺.

### Example 4: 3-[2-(4-chloro-3-fluorophenoxy)acetamido]-N-[(6-chloro-4-oxo-3,4-dihydro-2H-1-benzopyran-2-yl)methyl]bicyclo[1.1.1]pentane-1-carboxamide (Compound 103)

Modifying a reported benzylic oxidation procedure (U.S. Pat. Appl. Publ. (2004), US 20040224994 A1), to a mixture of Example 30 (0.019 g, 0.038 mmol) in CH₃CN (0.15 mL) and H₂O (0.15 mL) was added potassium persulfate (0.026 g, 0.095 mmol) and copper(II) sulfate pentahydrate (0.010 g, 0.038 mmol). The reaction mixture was heated to 80° C. for 20 minutes and then to 50° C. overnight. Then the reaction mixture was cooled to ambient temperature, diluted with H₂O (1 mL) and extracted with dichloromethane (3×5 mL). The combined organic extracts were dried over Na₂SO₄ and concentrated. The crude material was diluted with N,N-dimethylformamide, filtered, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.013 g, 0.026 mmol, 67% yield). ¹H NMR (400 MHz, DMSO-d₆) δ ppm 8.71 (s, 1H), 8.06 (t, J=5.9 Hz, 1H), 7.69-7.57 (m, 2H), 7.49 (t, J=8.9 Hz, 1H), 7.12-7.03 (m, 2H), 6.85 (ddd, J=9.0, 2.8, 1.2 Hz, 1H), 4.67-4.56 (m, 1H), 4.47 (s, 2H), 3.51 (dt, J=13.9, 6.1 Hz, 1H), 3.43-3.37 (m, 1H), 2.79 (dd, J=17.1, 12.1 Hz, 1H), 2.67 (dd, J=17.1, 3.5 Hz, 1H), 2.20 (s, 6H); MS (APCI⁺) m/z 507 (M+H)⁺.

### Example 5: 3-[2-(4-chloro-3-fluorophenoxy)acetamido]-N-{[rac-(2R,4R)-6-chloro-4-hydroxy-3,4-dihydro-2H-1-benzopyran-2-yl]methyl}bicyclo[1.1.1]pentane-1-carboxamide (Compound 104)

To a mixture of Example 4 (0.0076 g, 0.015 mmol) in methanol (0.27 mL) was added sodium borohydride (0.006 g, 0.26 mmol). This reaction mixture was allowed to stir at ambient temperature for 3 hours, was quenched with ammonium chloride (saturated aqueous solution, 1 mL) and extracted with ethyl acetate (3×5 mL). The combined organic layers were concentrated under heated N₂, diluted with N,N-dimethylformamide, and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.003 g, 0.006 mmol, 39% yield). ¹H NMR (500 MHz, DMSO-d₆, dr 17:1) δ ppm 8.73 (s, 1H), 8.03 (t, J=6.0 Hz, 1H), 7.50 (t, J=8.9 Hz, 1H), 7.37 (dd, J=2.7, 1.0 Hz, 1H), 7.29 (d, J=2.6 Hz, 0.6H), 7.20 (dd, J=8.7, 2.7 Hz, 0.6H), 7.14 (ddd, J=8.7, 2.7, 0.7 Hz, 1H), 7.08 (dd, J=11.4, 2.8 Hz, 1H), 6.85 (ddd, J=9.0, 2.9, 1.2 Hz, 1H), 6.81 (d, J=8.7 Hz, 0.6H), 6.74 (d, J=8.7 Hz, 1H), 5.66-5.59 (m, 1H), 4.74 (dd, J=10.7, 6.0 Hz, 1H), 4.47 (s, 2H), 4.19 (dtd, J=11.5, 5.8, 1.9 Hz, 1H), 3.44-3.38 (m, 1H), 3.27 (dt, J=13.6, 5.7 Hz, 1H), 2.20 (s, 0.36H), 2.20 (s, 6H), 2.15 (ddd, J=13.0, 6.1, 1.9 Hz, 1H), 1.53 (dt, J=13.0, 11.2 Hz, 1H); MS (APCI⁺) m/z 491 (M−H₂O+H)⁺.

### Example 6: (2R,4R)-6-chloro-4-hydroxy-N-[(1r,4R)-4-([5-(trifluoromethyl)pyridin-2-yl]methyl)carbamoyl)cyclohexyl]-3,4-dihydro-2H-1-benzopyran-2-carboxamide (Compound 105)

### Example 6A: Tert-Butyl ((1r,4r)-4-(((5-(trifluoromethyl)pyridin-2-yl)methyl)carbamoyl)cyclohexyl)carbamate

The reaction and purification conditions described in Example 2B substituting trans-4-((tert-butoxycarbonyl)amino)cyclohexanecarboxylic acid (ArkPharm) for the product of Example 1B, and (5-(trifluoromethyl)pyridin-2-yl)methanamine hydrochloride (PharmaBlock) for the product of Example 2A gave the title compound. MS (APCI⁺) m/z 402 (M+H)⁺.

### Example 6B: (R)-6-chloro-4-oxo-N-((1r,4R)-4-(((5-(trifluoromethyl)pyridin-2-yl)methyl)carbamoyl)cyclohexyl)chroman-2-carboxamide

The reaction and purification conditions described in Example 1C substituting the product of Example 6A for the product of Example 1A gave the title compound. MS (APCI⁺) m/z 510 (M+H)⁺.

### Example 6C: (2R,4R)-6-chloro-4-hydroxy-N-(1r,4R)-4-(((5-(trifluoromethyl)pyridin-2-yl)methyl)carbamoyl)cyclohexyl)-3,4-dihydro-2H-1-benzopyran-2-carboxamide

The product of Example 6B (24 mg, 0.047 mmol) was combined with methanol (1 mL), and the mixture was stirred at ambient temperature. Sodium borohydride (7.1 mg, 0.188 mmol) was added. After stirring for 30 minutes, saturated ammonium chloride solution (0.2 mL) was added, the resulting mixture was stirred for 10 minutes and then partitioned between dichloromethane (2×5 mL) and aqueous sodium carbonate solution (1M, 5 mL). The organic phases were combined, dried over sodium sulfate, and concentrated under reduced pressure. The resulting residue was taken up in methanol (1 mL) and filtered through a glass microfiber frit. The filtrate was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (16 mg, 0.031 mmol, 66% yield). ¹H NMR (400 MHz, DMSO-d₆) δ ppm 8.91-8.86 (m, 1H), 8.49 (t, J=6.0 Hz, 1H), 8.17 (dd, J=8.3, 2.4 Hz, 1H), 7.89 (d, =8.2 Hz, 1H), 7.46 (d, J=8.3 Hz, 1H), 7.39 (dd, J=2.8, 1.0 Hz, 1H), 7.20 (dd, J=8.7, 2.7 Hz, 1H), 6.89 (d, J=8.7 Hz, 1H), 5.70 (br s, 1H), 4.81 (dd, J=10.7, 5.9 Hz, 1H), 4.61 (dd, J=11.9, 2.2 Hz, 1H), 4.43 (d, J=5.8 Hz, 2H), 3.64-3.56 (m, 1H), 2.35 (ddd, J=12.8, 5.9, 2.3 Hz, 1H), 2.20 (tt, J=11.9, 3.2 Hz, 1H), 1.88-1.78 (m, 4H), 1.72 (td, J=12.3, 10.7 Hz, 1H), 1.54-1.24 (m, 4H); MS (APCI⁺) m/z 512 (M+H)⁺.

### Example 7: (2R)-6-chloro-4-oxo-N-[4-({[5-(trifluoromethyl)pyridin-2-yl]methyl}carbamoyl)bicyclo[2.2.2]octan-1-yl]-3,4-dihydro-2H-1-benzopyran-2-carboxamide (Compound 106)

### Example 7A: (2R)-4-amino-N-((5-(trifluoromethyl)pyridin-2-yl)methyl)bicyclo[2.2.2]octane-1-carboxamide, Trifluoroacetic Acid

(5-(Trifluoromethyl)pyridin-2-yl)methanamine hydrochloride (Pharma Block 53 mg, 0.25 mmol), 4-((tert-butoxycarbonyl)amino)bicyclo[2.2.2]octane-1-carboxylic acid (Ark Pharm, 67 mg, 0.25 mmol), and triethylamine (0.104 mL) were combined with N,N-dimethylformamide (5 mL) and stirred at ambient temperature. 1-[Bis(dimethylamino)methylene]-1H-1,2,3-triazolo[4,5-b]pyridinium 3-oxid hexafluorophosphate (HATU, 104 mg, 0.274 mmol) was added. After stirring at ambient temperature for 2 hours, the reaction mixture was partitioned between dichloromethane (3×25 mL) and aqueous sodium carbonate (1.0 M, 20 mL). The organic layers were combined, dried over anhydrous sodium sulfate, and concentrated under reduced pressure. The residue was taken up in dichloromethane (2 mL) and trifluoroacetic acid (0.019 mL, 0.25 mmol) was added in one portion. After stirring for 30 minutes, the reaction mixture was concentrated under reduced pressure, and the residue was directly purified by preparative HPLC [YMC TriArt™ Hybrid C18 5 μm ODS column, 50×100 mm, flow rate 140 mL/minute, 5-100% gradient of acetonitrile in buffer (0.1% trifluoroacetic acid)] to give the title compound (78 mg, 0.18 mmol, 71% yield). MS (APCI⁺) m/z 328 (M+H)⁺.

### Example 7B: (2R)-6-chloro-4-oxo-N-[4-({[5-(trifluromethyl)pyridin-2-yl]methyl}carbamoyl)bicyclo[2.2.2]octan-1-yl]-3,4-dihydro-2H-1-benzopyran-2-carboxamide

The reaction and purification conditions described in Example 2B substituting the product of Example 7A for the product of Example 2A gave the title compound. ¹H NMR (400 MHz, DMSO-d₆) δ ppm 8.88-8.85 (m, 1H), 8.20-8.13 (m, 2H), 7.71 (s, 1H), 7.66-7.58 (m, 2H), 7.37 (d, J=8.3 Hz, 1H), 7.15 (dd, J=8.7, 0.6 Hz, 1H), 5.06 (dd, J=8.3, 4.9 Hz, 1H), 4.40 (d, J=5.8 Hz, 2H), 3.04-2.83 (m, 2H), 1.87-1.72 (m, 12H); MS (APCI⁺) m/z 536 (M+H)⁺.

### Example 8: (2R,4R)-6-chloro-4-hydroxy-N-[4-({[5-(trifluoromethyl)pyridin-2-yl]methyl}carbamoyl)bicyclo[2.2.2]octan-1-yl]-3,4-dihydro-2H-1-benzopyran-2-carboxamide (Compound 107)

The reaction and purification conditions described in Example 6C substituting the product of Example 7 for the product of Example 6B gave the title compound. ¹H NMR (400 MHz, DMSO-d₆) δ ppm 8.91-8.86 (m, 1H), 8.49 (t, J=6.0 Hz, 1H), 8.17 (dd, J=8.3, 2.4 Hz, 1H), 7.89 (d, J=8.2 Hz, 1H), 7.46 (d, J=8.3 Hz, 1H), 7.39 (dd, J=2.8, 1.0 Hz, 1H), 7.20 (dd, J=8.7, 2.7 Hz, 1H), 6.89 (d, J=8.7 Hz, 1H), 5.70 (br s, 1H), 4.81 (dd, J=10.7, 5.9 Hz, 1H), 4.61 (dd, J=11.9, 2.2 Hz, 1H), 4.43 (d, J=5.8 Hz, 2H), 3.64-3.56 (m, 1H), 2.35 (ddd, J=12.8, 5.9, 2.3 Hz, 1H), 2.20 (tt, J=11.9, 3.2 Hz, 1H), 1.88-1.78 (m, 4H), 1.72 (td, J=12.3, 10.7 Hz, 1H), 1.54-1.24 (m, 4H); MS (APCI⁺) m/z 538 (M+H)⁺.

### Example 9: (2R)-6-chloro-N-(3-{5-[(4-chloro-3-fluorophenoxy)methyl]-1,3,4-oxadiazol-2-yl}bicyclo[1.1.1]pentan-1-yl)-4-oxo-3,4-dihydro-2H-1-benzopyran-2-carboxamide (Compound 108)

The reaction and purification conditions described in Example 2B substituting 3-(5-((4-chloro-3-fluorophenoxy)methyl)-1,3,4-oxadiazol-2-yl)bicyclo[1.1.1]pentan-1-amine (prepared as described in International Patent Publication WO2017/193030 A1) for the product of Example 2A gave the title compound. ¹H NMR (400 MHz, DMSO-d₆) δ ppm 9.15 (s, 1H), 7.69-7.61 (m, 2H), 7.53 (t, J=8.8 Hz, 1H), 7.25 (dd, J=11.3, 2.9 Hz, 1H), 7.21-7.14 (m, 1H), 6.97 (ddd, J=9.0, 2.9, 1.2 Hz, 1H), 5.43 (s, 2H), 5.13 (dd, J=7.7, 6.6 Hz, 1H), 3.00-2.94 (m, 2H), 2.49 (s, 6H) MS (APCI⁺) m/z 518 (M+H)⁺.

### Example 10: (2S)-6-chloro-N-(3-{5-[(4-chloro-3-fluorophenoxy)methyl]-1,3,4-oxadiazol-2-yl}bicyclo[1.1.1]pentan-1-yl)-4-oxo-3,4-dihydro-2H-1-benzopyran-2-carboxamide (Compound 109)

### Example 10A: (S)-6-chloro-4-oxochroman-2-carboxylic Acid

Chiral SFC purification as described in Example 1B also gave this title compound as the later eluting fraction. MS (ESI⁻) m/z 225 (M−H)⁻.

### Example 10B: (2S)-6-chloro-N-(3-{5-[(4-chloro-3-fluorophenoxy)methyl]-1,3,4-oxadiazol-2-yl}bicyclo[1.1.1]pentan-1-yl)-4-oxo-3,4-dihydro-2H-1-benzopyran-2-carboxamide

The reaction and purification conditions described in Example 2B substituting 3-(5-((4-chloro-3-fluorophenoxy)methyl)-1,3,4-oxadiazol-2-yl)bicyclo[1.1.1]pentan-1-amine for the product of Example 2A, and the product of Example 10A for the product of Example 1B gave the title compound. ¹H NMR (400 MHz, DMSO-d₆) δ ppm 9.15 (s, 1H), 7.68-7.62 (m, 2H), 7.53 (t, J=8.8 Hz, 1H), 7.24 (dd, J=11.2, 2.9 Hz, 1H), 7.20-7.15 (m, 1H), 6.97 (ddd, J=8.9, 2.9, 1.2 Hz, 1H), 5.43 (s, 2H), 5.13 (dd, J=7.7, 6.6 Hz, 1H), 3.02-2.94 (m, 2H), 2.49 (s, 6H); MS (APCI⁺) m/z 518 (M+H)⁺.

### Example 11: (2R,4R)-6-chloro-N-(3-{5-[(4-chloro-3-fluorophenoxy)methyl]-1,3,4-oxadiazol-2-yl}bicyclo[1.1.1]pentan-1-yl)-4-hydroxy-3,4-dihydro-2H-1-benzopyran-2-carboxamide (Compound 110)

The reaction and purification conditions described in Example 6C substituting the product of Example 9 for the product of Example 6B gave the title compound. ¹H NMR (400 MHz, DMSO-d₆) δ ppm 8.89 (s, 1H), 7.53 (t, J=8.8 Hz, 1H), 7.41-7.36 (m, 1H), 7.25 (dd, J=11.2, 3.0 Hz, 1H), 7.21 (ddd, J=8.7, 2.7, 0.7 Hz, 1H), 6.97 (ddd, J=9.0, 2.9, 1.2 Hz, 1H), 6.89 (d, J=8.7 Hz, 1H), 5.71 (d, J=6.2 Hz, 1H), 5.43 (s, 2H), 4.82 (dt, J=11.4, 6.0 Hz, 1H), 4.63 (dd, J=11.9, 2.3 Hz, 1H), 2.51 (s, 6H), 2.37 (ddd, J=12.9, 5.9, 2.4 Hz, 1H), 1.78-1.65 (m, 1H); MS (APCI⁺) m/z 502 (M−H₂O+H)⁺.

### Example 12: (2S,4S)-6-chloro-N-(3-{5-[(4-chloro-3-fluorophenoxy)methyl]-1,3,4-oxadiazol-2-yl}bicyclo[1.1.1]pentan-1-yl)-4-hydroxy-3,4-dihydro-2H-1-benzopyran-2-carboxamide (Compound 111)

The reaction and purification conditions described in Example 6C substituting the product of Example 10 for the product of Example 6B gave the title compound. ¹H NMR (500 MHz, DMSO-d₆) δ ppm 8.90 (s, 1H), 7.53 (t, J=8.9 Hz, 1H), 7.39 (dd, J=2.6, 1.0 Hz, 1H), 7.25 (dd, J=11.3, 2.9 Hz, 1H), 7.21 (ddd, J=8.8, 2.7, 0.7 Hz, 1H), 6.97 (ddd, J=9.0, 2.9, 1.2 Hz, 1H), 6.89 (d, J=8.7 Hz, 1H), 5.72 (d, J=6.2 Hz, 1H), 5.43 (s, 2H), 4.82 (dt, J=11.4, 6.0 Hz, 1H), 4.63 (dd, J=12.0, 2.3 Hz, 1H), 2.51 (s, 6H), 2.37 (ddd, J=12.9, 5.9, 2.3 Hz, 1H), 1.76-1.66 (m, 1H); MS (APCI⁺) m/z 502 (M−H₂O+H)⁺.

### Example 13: 2-(4-chloro-3-fluorophenoxy)-N-[(2S)-2-hydroxy-4-(2-{[(1s,3R)-3-(trifluoromethoxy)cyclobutyl]oxy}acetamido)bicyclo[2.2.2]octan-1-yl]acetamide (Compound 112)

### Example 13A: Ethyl 1,4-dioxaspiro[4.5]decane-8-carboxylate

A mixture of ethyl 4-oxocyclohexanecarboxylate (11.70 mL, 73.4 mmol), ethane-1,2-diol (12.29 mL, 220 mmol), and p-toluenesulfonic acid monohydrate (1.397 g, 7.34 mmol) in toluene (200 mL) was stirred at reflux with a Dean-Stark trap apparatus for 180 minutes. The reaction mixture was neutralized with N-ethyl-N-isopropylpropan-2-amine and then concentrated. The residue was purified on silica gel (0-30% ethyl acetate in heptane) to give 12.77 g of the title compound. ¹H NMR (400 MHz, DMSO-d₆) δ ppm 4.01 (q, J=7.1 Hz, 2H), 3.81 (s, 4H), 2.32 (tt, J=10.4, 3.8 Hz, 1H), 1.83-1.71 (m, 2H), 1.66-1.57 (m, 1H), 1.62-1.38 (m, 5H), 1.13 (t, J=7.1 Hz, 3H).

### Example 13B: Ethyl 8-acetyl-1,4-dioxaspiro[4.5]decane-8-carboxylate

To a solution of diisopropylamine (5.19 mL, 36.4 mmol) in tetrahydrofuran (25 mL) at 0° C. was added n-butyllithium slowly below 5° C. After stirring for 30 minutes, the solution was cooled to −78° C. under nitrogen, and a solution of Example 13A (6.0 g, 28.0 mmol) in tetrahydrofuran (3 mL) was added slowly, and the resultant mixture was stirred for 30 minutes at the same temperature. Then acetyl chloride (2.59 mL, 36.4 mmol) was added slowly to maintain the temperature below −60° C., and the mixture was stirred at −70° C. for 2 hours. The reaction was quenched with saturated NH₄Cl solution, and the aqueous phase was extracted with ethyl acetate. The organic layer was washed with brine, dried over magnesium sulfate and filtered. The filtrate was concentrated, and the residue was purified on silica gel (0-70% ethyl acetate in heptane) to give 6.78 g of the title compound. ¹H NMR (500 MHz, DMSO-d₆) δ ppm 4.19-4.11 (m, 2H), 3.85 (s, 4H), 2.13 (s, 3H), 2.10-2.01 (m, 2H), 1.90 (ddd, J=13.9, 9.6, 4.6 Hz, 2H), 1.54 (th, J=13.6, 4.7 Hz, 4H), 1.18 (dd, J=7.6, 6.5 Hz, 3H).

### Example 13C: Ethyl 1-acetyl-4-oxocyclohexane-1-carboxylate

A mixture of Example 13B (6.5 g, 25.4 mmol) and HCl (21.13 mL, 127 mmol) in acetone (60 mL) was stirred at ambient temperature overnight. Volatiles were removed under reduced pressure, and the residue was partitioned between water and dichloromethane. The organic layer was washed with brine, dried over magnesium sulfate and filtered. The filtrate was concentrated to give 5.46 g of the title compound which was used without further purification. ¹H NMR (400 MHz, DMSO-d₆) δ ppm 4.16 (q, J=7.1 Hz, 2H), 2.17 (s, 3H), 2.35-2.07 (m, 8H), 1.17 (t, J=7.1 Hz, 3H).

### Example 13D: Ethyl 4-(benzylamino)-2-oxobicyclo[2.2.2]octane-1-carboxylate, Hydrochloric Acid

A mixture of Example 13C (9.7 g, 45.7 mmol), benzylamine (14.98 mL, 137 mmol), and p-toluenesulfonic acid monohydrate (0.087 g, 0.457 mmol) in toluene (100 mL) was stirred at reflux with a Dean-Stark trap apparatus overnight. The mixture was concentrated, and the residue was stirred with a mixture of ethyl acetate (50 mL) and 3 N HCl (100 mL) for 30 minutes. The precipitate was collected by filtration, washed with mixture of ethyl acetate/heptane, and air-dried to give 11.3 g of the title compound as an HCl salt. The filtrate was neutralized with 6 N NaOH and extracted with ethyl acetate (100 mL×2). The organic layer was washed with brine, dried over magnesium sulfate and filtered. The residue was purified on silica gel (0-700% ethyl acetate in heptane) to give another 0.77 g of the title compound. ¹H NMR (400 MHz, DMSO-d₆) δ ppm 9.73 (t, J=6.2 Hz, 2H), 7.87-7.12 (m, 5H), 4.09 (m, 4H), 2.88 (s, 2H), 2.08 (dt, J=20.7, 13.4 Hz, 6H), 1.16 (t, J=7.1 Hz, 3H); MS (ESI⁺) m/z 302.1 (M+H)⁺.

### Example 13E: 4-(benzylamino)-2-oxobicyclo[2.2.2]octane-1-carboxylic Acid Hydrochloride

A mixture of Example 13D (20.7 g, 61.3 mmol) and 25% aqueous sodium hydroxide (49.0 mL, 306 mmol) in methanol (200 mL) and water (200 mL) was stirred for 24 hours at ambient temperature. The mixture was concentrated, and the residue was acidified with 1 N HCl. The precipitate was collected by filtration, washed with water, and air dried to give 16.4 g of the title compound. ¹H NMR (400 MHz, DMSO-d₆) δ ppm 12.70 (s, 1H), 9.67 (s, 2H), 7.62 (dd, J=7.5, 2.0 Hz, 2H), 7.43 (d, J=6.6 Hz, 3H), 4.13 (s, 2H), 2.87 (s, 2H), 2.08 (tdq, J=14.4, 10.8, 5.8, 5.0 Hz, 8H).

### Example 13F: 1-amino-4-(benzylamino)bicyclo[2.2.2]octan-2-one, Trifluoroacetic Acid

To a mixture of Example 13E (5.0 g, 16.14 mmol) and oxalyl chloride (24.21 mL, 48.4 mmol) in dichloromethane (100 mL) was added N,N-dimethylformamide (0.250 mL, 3.23 mmol), and the suspension was stirred at ambient temperature for 14 hours. The mixture was concentrated, and the residue was triturated with ether/heptane. The precipitate was collected by filtration and dried to give 4.99 g of 4-(benzylamino)-2-oxobicyclo[2.2.2]octane-1-carbonyl chloride which was used in next step without further purification. To a mixture of sodium azide (0.832 g, 12.80 mmol) in dioxane (10 mL) and water (10 mL) at 0° C. was added a suspension of the crude 4-(benzylamino)-2-oxobicyclo[2.2.2]octane-1-carbonyl chloride (0.934 g, 3.2 mmol) in dioxane (30 mL), and the solution was stirred at ambient temperature for 30 minutes. Volatiles were removed to give the crude corresponding acyl azide which was suspended with 50 mL of toluene and heated at 65° C. for 2 hours to convert to the isocyanate, 4-(benzylamino)-1-isocyanatobicyclo[2.2.2]octan-2-one. Then 3 N HCl (40 mL) was added carefully, and the mixture was stirred at 100° C. for 3 hours. Volatiles were removed under vacuum, and the residue was stirred with methanol, and the inorganic salts were removed by filtration. The filtrate was concentrated, and the residue was purified by HPLC (0-60% acetonitrile in 0.1% trifluoroacetic acid/water on Phenomenex® C18 10 μm (250 mm×50 mm) column at a flow rate of 50 mL/minute) to give 550 mg of the title compound as a trifluoroacetate salt. ¹H NMR (400 MHz, DMSO-d₆) δ ppm 9.47 (s, 2H), 8.59 (s, 3H), 7.55-7.39 (m, 5H), 4.18 (s, 2H), 3.01 (s, 2H), 2.28-2.09 (m, 6H), 1.96 (td, J=12.6, 12.0, 7.0 Hz, 2H); MS (ESI⁺) m/z 245.1 (M+H)⁺.

### Example 13G: (S)-tert-butyl (4-(benzylamino)-2-hydroxybicyclo[2.2.2]octan-1-yl)carbamate, Hydrochloric Acid

Magnesium sulfate (0.196 g) and nicotinamide adenine dinucleotide phosphate (NADPH, 0.200 g) were mixed in 360 mL of potassium phosphate buffer (125 mM, pH=7.0) and 0.04 L of isopropanol. A portion of this solution (60 mL) was reserved and used to dissolve Codexis® KRED-P2-C02 enzyme (400 mg). Example 13F (20.0 g) was added to the 340 mL of remaining buffered solution and the pH was adjusted to 7.5 with 50% (weight/weight) NaOH. The reaction was initiated by addition of the enzyme in the 60 mL of buffered solution. The reaction mixture was stirred overnight at 40° C. The cloudy, aqueous solution was adjusted to pH>11 with 50% weight/weight aqueous sodium hydroxide. Diatomaceous earth (20 g) was added to the reaction mixture and then stirred for 10 minutes. The mixture was filtered to remove all insoluble material. The aqueous layer was charged back to the reaction vessel and di-tert-butyl dicarbonate (16 g, 1.2 equivalent) in 400 mL of ethyl acetate was charged to the same vessel. The biphasic solution was stirred for two hours. The aqueous layer was routinely checked to maintain pH>10. At 2 hours, the two layers were separated, and the aqueous layer was charged back to the reaction vessel. The amount of amino alcohol intermediate remaining in the aqueous layer was determined by high performance liquid chromatography (HPLC: Supelco Acentis® Express C18 column, 4.6×150 mm, 2.7 micron. Mobile A=0.1% H₃PO₄ in water; Mobile B=85% acetonitrile-15% methanol. Wavelength=218 nm. Flow rate=1.25 mL/minute, 25° C. column temperature) and 1.2 equivalents of di-tert-butyl dicarbonate were added to the reaction vessel dissolved in ethyl acetate (200 mL). The pH was maintained >10. This reaction proceeded for 2 hours and the two layers were separated. The organic layers were combined, washed with brine containing 2.5% sodium hydroxide (60 mL), filtered through magnesium sulfate and concentrated in vacuo. The residual material was taken up in 200 mL methyl tert-butyl ether. The mixture was cooled to 5° C. and 4 N HCl in dioxane (14.0 mL) was slowly added. The precipitated material was collected by filtration and dried in vacuo to provide the title compound. (18.1 g, 75%). ¹H NMR (400 MHz, DMSO-d₆) δ ppm 9.28 (t, J=6.3 Hz, 2H), 7.69-7.55 (m, 2H), 7.48-7.30 (m, 3H), 6.23 (s, 1H), 5.18 (s, 1H), 4.03-3.98 (m, 3H), 2.40-2.26 (m, 1H), 2.11-1.64 (m, 9H), 1.37 (s, 9H); MS (APCI⁺) m/z 347.4 (M+H)⁺.

### Example 13H: (S)-Tert-Butyl (4-amino-2-hydroxybicyclo[2.2.2]octan-1-yl)carbamate, Hydrochloric Acid

To a mixture of Example 13G (29.75 g, 78 mmol) in methanol (96 mL) was added to 10% Pd(OH)₂/C wet, (3.15 g, 9.42 mmol) in a 600 mL stainless steel reactor. The reactor was purged with nitrogen, and then was stirred at 900 RPM under 50 psi of hydrogen at 50° C. for 18 hours. The reaction mixture was filtered, and the filtrate was concentrated to give the title compound. ¹H NMR (400 MHz, DMSO-d₆) δ ppm 8.09 (brs, 3H), 6.16 (s, 1H), 5.12 (d, J=4.2 Hz, 1H), 3.95 (dt, J=9.3, 3.2 Hz, 1H), 2.14 (ddd, J=12.7, 9.4, 3.0 Hz, 1H), 2.09 1.97 (m, 1H), 1.92-1.52 (m, 8H), 1.36 (s, 9H); MS (+ESI) m/z 257.1 (M+H).

### Example 13I: (S)-allyl (4-amino-3-hydroxybicyclo[2.2.2]octan-1-yl)carbamate, Hydrochloric Acid

To a suspension of Example 13H (15.00 g, 51.2 mmol) and sodium carbonate (16.29 g, 154 mmol) in tetrahydrofuran (150 mL) and water (75 mL) at 0° C. was added allyl chloroformate (6.56 mL, 61.5 mmol). The mixture was stirred at 0° C. for 10 minutes and then warmed to ambient temperature and stirred for an additional 1.5 hours. The reaction was diluted with ethyl acetate (200 mL) and washed with water (150 mL), 1 N HCl (75 mL), water (75 mL), and brine (75 mL). The organic layer was dried over MgSO₄, filtered, concentrated, and triturated with heptane to give the crude (S)-allyl tert-butyl (2-hydroxybicyclo[2.2.2]octane-1,4-diyl)dicarbamate which was used in the next step without further purifications. This crude material was dissolved in methanol (110 mL), a 4 N dioxane solution of HCl (21.15 mL, 85 mmol) was added, and the mixture was stirred at 50° C. for 1 hour. Volatiles were removed under vacuum. The residue was triturated in tert-butyl methyl ether (50 mL), filtered, and vacuum oven-dried to provide the title compound, which was used in the next step without further purifications. ¹H NMR (400 MHz, DMSO-d₆) δ ppm 8.01 (s, 3H), 7.03 (s, 1H), 5.88 (ddt, J=17.2, 10.6, 5.4 Hz, 1H), 5.57 (d, J=4.7 Hz, 1H), 5.26 (dq, J=17.2, 1.8 Hz, 1H), 5.16 (dq, J=10.4, 1.6 Hz, 1H), 4.40 (d, J=5.3 Hz, 2H), 3.84 (ddt, J=9.4, 4.9, 2.7 Hz, 1H), 2.22 (ddd, J=13.0, 9.5, 3.0 Hz, 1H), 2.05-1.95 (m, 1H), 1.93-1.53 (m, 8H); MS (DCI⁺) m/z 241.2 (M+H)⁺.

### Example 13J: (S)-Allyl (4-(2-(4-chloro-3-fluorophenoxy)acetamido)-3-hydroxybicyclo[2.2.2]octan-1-yl)carbamate

To a suspension of Example 13I (11 g, 39.7 mmol) and 2-(4-chloro-3-fluorophenoxy)acetic acid (9.76 g, 47.7 mmol) in dimethylformamide (100 mL) was added triethylamine (16.62 mL, 119 mmol) followed by HATU (18.14 g, 47.7 mmol). The mixture was stirred for 90 minutes, diluted with water (300 mL), and extracted with ethyl acetate (300, 150 mL). The combined organic layers were washed with brine and concentrated. The concentrate was dissolved in methanol (30 mL) and tetrahydrofuran (60 mL) and treated with a solution of lithium hydroxide (1.428 g, 59.6 mmol) in water (20 mL). The mixture was stirred for 2 hours and then concentrated. The residue was dissolved in ethyl acetate (120 mL), washed with water (60 mL) and brine (100 mL), dried over MgSO₄, and filtered The filtrate was concentrated and flushed through a silica plug eluting with ethyl acetate/heptanes (9:1) to provide the title compound as a white solid. ¹H NMR (500 MHz, DMSO-d₆) δ ppm 7.48 (t, J=8.9 Hz, 1H), 7.25 (s, 1H), 7.05 (dd, J=11.4, 2.8 Hz, 1H), 6.94 (s, 1H), 6.83 (ddd, J=9.0, 2.9, 1.2 Hz, 1H), 5.88 (ddt, J=17.2, 10.6, 5.3 Hz, 1H), 5.26 (dq, J=17.2, 1.7 Hz, 1H), 5.16 (dq, J=10.5, 1.5 Hz, 1H), 5.05 (d, J=4.4 Hz, 1H), 4.46 (s, 2H), 4.40 (d, J=5.4 Hz, 2H), 4.06-3.98 (m, 1H), 2.18 (ddd, J=12.8, 9.5, 2.9 Hz, 1H), 2.10-1.97 (m, 1H), 1.95-1.64 (m, 8H); MS (+ESI) m/z 427.2 (M+H).

### Example 13K: (S)—N-(4-amino-2-hydroxybicyclo[2.2.2]octan-1-yl)-2-(4-chloro-3-fluorophenoxy)acetamide

To a solution of Example 13J (15.43 g, 36.1 mmol) and diethylamine (37.8 mL, 361 mmol) in dichloromethane (100 mL) was added tetrakis(triphenylphosphine)palladium(O) (0.835 g, 0.723 mmol). The mixture was stirred at ambient temperature for 3 hours. The reaction mixture concentrated, and the residue was purified on a 330 g column using the Biotage Isolera™ One flash system eluting with dichloromethane/methanol/30% ammonium hydroxide (10:1:0.1). The desired fractions were concentrated; the residue was dissolved in ethyl acetate with 2% methanol and concentrated until most of the solvents were removed.

To the warm remaining solution was added heptane. The resulting solution was cooled to room temperature, and a precipitate formed. The solids were collected by filtration and washed with ethyl acetate/heptanes (1:9). The precipitation process was repeated two more times. The solids were dried in a vacuum oven to provide 9.7 g of the title compound. ¹H NMR (400 MHz, DMSO-d₆) δ ppm 7.48 (t, J=8.9 Hz, 1H), 7.18 (s, 1H), 7.05 (dd, J=11.4, 2.9 Hz, 1H), 6.82 (ddd, J=8.9, 2.9, 1.2 Hz, 1H), 4.95 (d, J=4.3 Hz, 1H), 4.45 (s, 2H), 3.97 (dd, J=8.0, 3.5 Hz, 1H), 2.04 (ddd, J=13.1, 11.2, 4.8 Hz, 1H), 1.94-1.69 (m, 4H), 1.54-1.22 (m, 5H); MS (+ESI) m/z 343.3 (M+H).

### Example 13L: (cis)-3-(benzyloxy)cyclobutanol

To a solution of 3-(benzyloxy)cyclobutanone (1.0 g, 5.67 mmol) in methanol (10 mL), sodium tetrahydroborate (0.215 g, 5.67 mmol) was added portionwise at −30° C. over 10 minutes, and then the mixture was stirred at the same temperature for one hour. The reaction mixture was cooled with an ice bath, and saturated ammonium chloride solution was added carefully to quench the reaction. The volatiles were removed under vacuum. The residue was extracted with ethyl acetate. The organic layer was dried over magnesium sulfated and filtered. The filtrate was concentrated, and the residue was purified on silica gel (0-70% ethyl acetate in heptane) to give 0.75 g of the title compound. ¹H NMR (400 MHz, DMSO-d₆) δ ppm 7.38-7.23 (m, 5H), 4.33 (s, 2H), 3.68 (ddt, J=14.5, 7.9, 6.7 Hz, 1H), 3.60-3.48 (m, 1H), 2.59-2.49 (m, 2H), 1.73 (dtd, J=8.9, 7.8, 2.9 Hz, 2H).

### Example 13M: Tert-Butyl 2-((cis)-3-(benzyloxy))cyclobutoxy)acetate

To a solution of Example 13L (0.63 g, 3.53 mmol), tert-butyl 2-bromoacetate (0.783 mL, 5.30 mmol) and tetrabutylammonium hydrogen sulfate (0.060 g, 0.177 mmol) in toluene (10 mL) and water (0.3 mL), sodium hydroxide (2.121 g, 53.0 mmol) in 3 mL of water was added. The two-phase mixture was stirred at ambient temperature for 2 hours. The organic layer was diluted with more ethyl acetate, washed with water and brine, dried over magnesium sulfate and filtered. The filtrate was concentrated, and the residue was purified on silica gel (0-60% ethyl acetate in heptane) to give 0.95 g of the title compound. ¹H NMR (400 MHz, DMSO-d₆) δ ppm 7.39-7.23 (m, 5H), 4.35 (s, 2H), 3.86 (s, 2H), 3.71-3.58 (m, 2H), 2.56 (dtd, J=9.4, 6.6, 2.9 Hz, 2H), 1.79 (dtd, J=9.2, 7.6, 2.9 Hz, 2H), 1.41 (s, 9H).

### Example 13N: Ter-Butyl 2-((cis)-3-hydroxycyclobutoxy)acetate

To a solution of Example 13M (0.94 g, 3.22 mmol) in tetrahydrofuran (8 mL) in a 20 mL Barnstead Hast C reactor was added 5% Pd/C, wet (0.1 g, 0.470 mmol), and the reaction mixture was stirred at 50° C. and 78 psi of hydrogen for 4 hours. The suspension was filtered, and the filtrate was concentrated under vacuum to give 0.67 g of the title compound which was used without further purification. ¹H NMR (400 MHz, DMSO-d₆) δ ppm 4.99 (d, J=6.6 Hz, 1H), 3.83 (s, 2H), 3.64 (ddt, J=14.5, 7.9, 6.6 Hz, 1H), 3.53 (tt, J=7.9, 6.5 Hz, 1H), 2.51-2.44 (m, 2H), 1.78-1.65 (m, 2H), 1.41 (s, 9H).

### Example 13O: Tert-Butyl 2-((cis)-3-(trifluoromethoxy)cyclobutoxy)acetate

To a mixture of silver(I) trifluoromethanesulfonate (2.52 g, 9.79 mmol), 1-chloromethyl-4-fluoro-1,4-diazoniabicyclo[2.2.2]octane bis(tetrafluoroborate) (1.734 g, 4.89 mmol), and potassium fluoride (0.758 g, 13.05 mmol) in a flask wrapped with aluminum foil and cooled with a water bath, Example 13N (0.66 g, 3.26 mmol) in ethyl acetate (25 mL) was added, followed by 2-fluoropyridine (0.841 mL, 9.79 mmol) and trimethyl(trifluoromethyl)silane (4.89 mL, 9.79 mmol) dropwise to keep the internal temperature lower than 30° C. The reaction mixture was stirred at ambient temperature overnight. The suspension was filtered through a diatomaceous earth cartridge and washed with more ethyl acetate. The organic filtrate was dried over magnesium sulfate and filtered. The filtrate was concentrated, and the residue was purified on silica gel (0-70% ethyl acetate in heptane) to give 0.46 g of the title compound. ¹H NMR (400 MHz, DMSO-d₆) δ ppm 4.46 (p, J=7.1 Hz, 1H), 3.92 (s, 2H), 3.79-3.67 (m, 1H), 2.74 (dtt, J=9.2, 5.7, 2.8 Hz, 2H), 2.15-2.03 (m, 2H), 1.42 (s, 9H).

### Example 13P: 2-((cis)-3-(trifluoromethoxy)cyclobutoxy)acetic Acid

A mixture of Example 13O (0.46 g, 1.702 mmol) and 2,2,2-trifluoroacetic acid (3.93 mL, 51.1 mmol) in dichloromethane (5.0 mL) was stirred at ambient temperature for 3 hours. Solvent and excess trifluoroacetic acid were removed under high vacuum to give 0.36 g of the title compound which was used without further purification. ¹H NMR (400 MHz, DMSO-d₆) δ ppm 12.63 (s, 1H), 4.47 (p, J=7.1 Hz, 1H), 3.95 (s, 2H), 3.81-3.70 (m, 1H), 2.75 (tdt, J=9.0, 5.7, 2.4 Hz, 2H), 2.15-2.03 (m, 2H).

### Example 13Q: 2-(4-chloro-3-fluorophenoxy)-N-[(2S)-2-hydroxy-4-(2-{[(1s,3R)-3-(trifluoromethoxy)cyclobutyl]oxy}acetamido)bicyclo[2.2.2]octan-1-yl]acetamide

To a mixture of Example 13K (52 mg, 0.152 mmol), Example 13P (34.1 mg, 0.159 mmol), and N-ethyl-N-isopropylpropan-2-amine (0.106 mL, 0.607 mmol) in N,N-dimethylformamide (2.0 mL), 2-(3H-[1,2,3]triazolo[4,5-b]pyridin-3-yl)-1,1,3,3-tetramethylisouronium hexafluorophosphate(V) (72.1 mg, 0.190 mmol) was added, and the mixture was stirred at ambient temperature for 1 hour. Volatiles were removed under high vacuum, and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 67 mg of the title compound. ¹H NMR (400 MHz, DMSO-d₆) δ ppm 7.48 (t, J=8.9 Hz, 1H), 7.25 (s, 1H), 7.09-6.97 (m, 2H), 6.83 (dd, J=9.0, 2.7 Hz, 1H), 4.47 (p, J=7.1 Hz, 1H), 4.46 (s, 2H), 4.03 (dd, J=9.7, 3.1 Hz, 1H), 3.69 (p, J=6.9 Hz, 1H), 3.68 (s, 2H), 2.72 (dtt, J=9.2, 5.8, 2.9 Hz, 2H), 2.26 (ddd, J=12.5, 9.5, 2.4 Hz, 1H), 2.12 (dp, J=9.6, 3.6 Hz, 2H), 2.11-2.00 (m, 1H), 1.97-1.72 (m, 8H); MS (APCI⁺) m/z 539.1 (M+H).

### Example 14: 6-chloro-4-oxo-N-[3-(2-{[(1s,3s)-3-(trifluoromethoxy)cyclobutyl]oxy}acetamido)bicyclo[1.1.1]pentan-1-yl]-3,4-dihydro-2H-1-benzopyran-2-carboxamide (Compound 113)

### Example 14A: Tert-Butyl (3-(6-chloro-4-oxochroman-2-carboxamido)bicyclo[1.1.1]pentan-1-yl)carbamate

The reaction and purification conditions described in Example 2B substituting 6-chloro-4-oxochroman-2-carboxylic acid (Princeton Bio) for the product of Example 1B, and tert-butyl (3-aminobicyclo[1.1.1]pentan-1-yl)carbamate (PharmaBlock) for the product of Example 2A gave the title compound. MS (ESI⁻) m/z 405 (M−H)⁻.

### Example 14B: N-(3-aminobicyclo[1.1.1]pentan-1-yl)-6-chloro-4-oxochroman-2-carboxamide, Trifluoroacetic Acid

The product of Example 14A (600 mg, 1.48 mmol) was stirred in dichloromethane (2 mL) at ambient temperature. Trifluoroacetic acid (1 mL) was added in one portion. After stirring for 30 minutes, the reaction mixture was concentrated under reduced pressure to give the title compound (0.63 g, 1.50 mmol, 102% yield). MS (ESI⁺) (m/z 307 (M+H)⁺.

### Example 14C: 6-chloro-4-oxo-N-[3-(2-{[(1s,3s)-3-(trifluoromethoxy)cyclobutyl]oxy}acetamido)bicyclo[1.1.1]pentan-1-yl]-3,4-dihydro-2H-1-benzopyran-2-carboxamide

The reaction and purification conditions described in Example 2B substituting the product of Example 14B for the product of Example 2A, and the product of Example 13P for the product of Example 1B gave the title compound. ¹H NMR (400 MHz, DMSO-d₆) δ ppm 8.92 (s, 1H), 8.36 (s, 1H), 7.68-7.60 (m, 2H), 7.20-7.12 (m, 1H), 5.08 (t, J=7.1 Hz, 1H), 4.47 (p, J=7.1 Hz, 1H), 3.72 (s, 2H), 3.75-3.63 (m, 1H), 2.94 (d, J=7.1 Hz, 2H), 2.79-2.67 (m, 2H), 2.23 (s, 6H), 2.19-2.08 (m, 2H); MS (APCI⁺) m/z 503 (M+H)⁺.

### Example 15: rac-(2R,4R)-6-chloro-4-hydroxy-N-[3-(2-{[(1s,3s)-3-(trifluoromethoxy)cyclobutyl]oxy}acetamido)bicyclo[1.1.1]pentan-1-yl]-3,4-dihydro-2H-1-benzopyran-2-carboxamide (Compound 114)

The reaction and purification conditions described in Example 6C substituting the product of Example 14C for the product of Example 6B gave the title compound. ¹H NMR (400 MHz, DMSO-d₆) δ ppm 8.66 (s, 1H), 8.36 (s, 1H), 7.37 (dd, J=2.7, 1.0 Hz, 1H), 7.19 (dd, J=8.7, 2.7 Hz, 1H), 6.88 (d, J=8.7 Hz, 1H), 5.84-5.57 (m, 1H), 4.80 (dd, J=10.7, 5.9 Hz, 1H), 4.58 (dd, J=12.1, 2.3 Hz, 1H), 4.48 (p, J=7.2 Hz, 1H), 3.72 (s, 2H), 3.74-3.64 (m, 1H), 2.79-2.68 (m, 2H), 2.36-2.30 (m, 1H), 2.25 (s, 6H), 2.20-2.09 (m, 2H), 1.75-1.60 (m, 1H); MS (APCI⁺) m/z 487 (M−H₂O+H)⁺.

### Example 16: (2R)-6-chloro-N-[(3S)-3-hydroxy-4-(2-{[(1s,3R)-3-(trifluoromethoxy)cyclobutyl]oxy}acetamido)bicyclo[2.2.2]octan-1-yl]-4-oxo-3,4-dihydro-2H-1-benzopyran-2-carboxamide (Compound 115)

### Example 16A: Tert-Butyl ((S)-4-((R)-6-chloro-4-oxochroman-2-carboxamido)-2-hydroxybicyclo[2.2.2]octan-1-yl)carbamate

The reaction and purification conditions described in Example 2B substituting the product of Example 13H for the product of Example 2A gave the title compound. MS (ESI⁺) m/z 409 (M−C(CH₃)₃+H)⁺.

### Example 16B: (2R)-6-chloro-N-[(3S)-3-hydroxy-4-(2-{[(1s,3R)-3-(trifluoromethoxy)cyclobutyl]oxy}acetamido)bicyclo[2.2.2]octan-1-yl]-4-oxo-3,4-dihydro-2H-1-benzopyran-2-carboxamide

The reaction and purification conditions described in Example 1C substituting the product of Example 16A for the product of Example 1A, and the product of Example 13P for the product of Example 1B gave the title compound. ¹H NMR (400 MHz, DMSO-d₆) δ ppm 7.71 (s, 1H), 7.66-7.57 (m, 2H), 7.15 (d, J=8.6 Hz, 1H), 6.92 (s, 1H), 5.19 (d, J=4.6 Hz, 1H), 5.04 (dd, J=8.2, 5.0 Hz, 1H), 4.47 (p, J=7.1 Hz, 1H), 3.95-3.88 (m, 1H), 3.77-3.64 (m, 3H), 2.99-2.85 (m, 2H), 2.74 (dtd, J=9.9, 6.7, 3.3 Hz, 2H), 2.28-2.16 (m, 2H), 2.11 (d, J=9.6 Hz, 2H), 1.95-1.81 (m, 3H), 1.78-1.66 (m, 5H); MS (APCI⁺) m/z 561 (M+H)⁺.

### Example 17: 2-(4-chlorophenoxy)-N-[4-(2-{[(1s,3s)-3-(trifluoromethoxy)cyclobutyl]oxy}acetamido)bicyclo[2.2.2]octan-1-yl]acetamide (Compound 116)

### Example 17A: N-(4-aminobicyclo[2.2.2]octan-1-yl)-2-(4-chlorophenoxy)acetamide, 2 Trifluoroacetic Acid

The reaction and purification conditions described in Examples 14A through 14B substituting 2-(4-chlorophenoxy)acetic acid for 6-chloro-4-oxochroman-2-carboxylic acid, and tert-butyl (4-aminobicyclo[2.2.2]octan-1-yl)carbamate for tert-butyl (3-aminobicyclo[1.1.1]pentan-1-yl)carbamate gave the title compound. MS (ESI⁻) m/z 407 (M−H)⁻.

### Example 17B: 2-(4-chlorophenoxy)-N-[4-(2-{[(1s,3s)-3-(trifluoromethoxy)cyclobutyl]oxy}acetamido)bicyclo[2.2.2]octan-1-yl]acetamide

The reaction and purification conditions described in Example 2B substituting the product of Example 17A for the product of Example 2A, and the product of Example 13P for the product of Example 1B gave the title compound. ¹H NMR (500 MHz, DMSO-d₆) δ ppm 7.43 (s, 1H), 7.36-7.29 (m, 2H), 6.98 (s, 1H), 6.96-6.89 (m, 2H), 4.47 (p, J=7.1 Hz, 1H), 4.38 (s, 2H), 3.68 (p, J=6.9 Hz, 1H), 3.68 (s, 2H), 2.77-2.68 (m, 2H), 2.16-2.07 (m, 2H), 1.89 (s, 12H); MS (APCI⁺) m/z 505 (M+H)⁺.

### Example 18: (2R,4R)-6-chloro-4-hydroxy-N-[(3S)-3-hydroxy-4-(2-{[(1s,3R)-3-(trifluoromethoxy)cyclobutyl]oxy}acetamido)bicyclo[2.2.2]octan-1-yl]-3,4-dihydro-2H-1-benzopyran-2-carboxamide (Compound 117)

The reaction and purification conditions described in Example 6C substituting the product of Example 16B for the product of Example 6B gave the title compound. ¹H NMR (400 MHz, DMSO-d₆) δ ppm 7.37 (dd, J=2.7, 0.9 Hz, 1H), 7.34 (s, 1H), 7.18 (dd, J=8.7, 2.7 Hz, 1H), 6.94 (s, 1H), 6.86 (d, J=8.7 Hz, 1H), 5.66 (br s, 1H), 5.22 (br s, 1H), 4.77 (dd, J=10.6, 5.9 Hz, 1H), 4.55 (dd, J=11.8, 2.2 Hz, 1H), 4.48 (p, J=7.1 Hz, 1H), 3.94 (dd, J=9.5, 3.3 Hz, 1H), 3.78-3.65 (m, 3H), 2.81-2.69 (m, 2H), 2.36-2.19 (m, 3H), 2.18-2.07 (m, 2H), 2.02-1.65 (m, 9H); MS (APCI⁺) m/z 545 (M−H₂O+H)⁺.

### Example 19: (1s,3s)-N-{3-[2-(4-chloro-3-fluorophenoxy)acetamido]bicyclo[1.1.1]pentan-1-yl}-3-(trifluoromethoxy)cyclobutane-1-carboxamide (Compound 118)

The reaction and purification conditions described in Example 2B substituting N-(3-aminobicyclo[1.1.1]pentan-1-yl)-2-(4-chloro-3-fluorophenoxy)acetamide (prepared as described in International Patent Publication WO2017/193034 A1) for the product of Example 2A, and the product of Example 250 for the product of Example 1B gave the title compound. ¹H NMR (400 MHz, DMSO-d₆) δ ppm 8.69 (s, 1H), 8.52 (s, 1H), 7.49 (t, J=8.9 Hz, 1H), 7.07 (dd, J=11.4, 2.8 Hz, 1H), 6.85 (ddd, J=8.9, 2.9, 1.2 Hz, 1H), 4.73 (p, J=7.5 Hz, 1H), 4.47 (s, 2H), 2.60-2.51 (m, 1H), 2.43 (dtd, J=10.2, 7.2, 2.9 Hz, 2H), 2.29-2.17 (m, 2H), 2.22 (s, 6H); MS (APCI⁺) m/z 451 (M+H)⁺.

### Example 20: (2R,4R)-6-chloro-4-hydroxy-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide (Compound 119)

The reaction and purification conditions described in Example 6C substituting the product of Example 33B for the product of Example 6B gave the title compound. ¹H NMR (400 MHz, DMSO-d₆) δ ppm 8.92-8.87 (m, 1H), 8.70 (s, 1H), 8.54 (t, J=6.0 Hz, 1H), 8.19 (dd, J=8.3, 2.4 Hz, 1H), 7.45 (d, J=8.3 Hz, 1H), 7.38 (dd, J=2.7, 0.9 Hz, 1H), 7.20 (ddd, J=8.8, 2.7, 0.7 Hz, 1H), 6.88 (d, J=8.7 Hz, 1H), 5.70 (s, 1H), 4.85-4.76 (m, 1H), 4.60 (dd, J=12.0, 2.3 Hz, 1H), 4.43 (d, J=6.0 Hz, 2H), 2.36 (ddd, J=12.9, 5.9, 2.3 Hz, 1H), 2.25 (s, 6H), 1.77-1.63 (m, 1H); MS (APCI⁺) m/z 496 (M+H)⁺.

### Example 21: 2-(4-chloro-3-fluorophenoxy)-N-{(3R,6S)-6-[3-(4-chlorophenoxy)azetidine-1-carbonyl]oxan-3-yl}acetamide (Compound 120)

### Example 21A: Tert-Butyl ((3R,6S)-6-(3-(4-chlorophenoxy)azetidine-1-carbonyl)tetrahydro-2H-pyran-3-yl)carbamate

The methodologies described in Example 30D substituting (2S,5R)-5-((tert-butoxycarbonyl)amino)tetrahydro-2H-pyran-2-carboxylic acid (purchased from Astatech) for 3-(2-(4-chloro-3-fluorophenoxy)acetamido)bicyclo[1.1.1]pentane-1-carboxylic acid and substituting 3-(4-chlorophenoxy)azetidine (purchased from PharmaBlock) for Example 30C gave the title compound. MS (APCI⁺) m/z 411 (M+H)⁺.

### Example 21B: ((2S,5R)-5-aminotetrahydro-2H-pyran-2-yl)(3-(4-chlorophenoxy)azetidin-1-yl)methanone

To a solution of Example 21A (0.045 g, 0.110 mmol) in dichloromethane (0.11 mL) was added trifluoroacetic acid (0.06 mL, 0.77 mmol). The reaction mixture stirred for 1 hour and was concentrated to afford the title compound which was carried forward without further purification. MS (APCI⁺) m/z 311 (M+H)⁺.

### Example 21C: 2-(4-chloro-3-fluorophenoxy)-N-{(3R,6S)-6-[3-(4-chlorophenoxy)azetidine-1-carbonyl]oxan-3-yl}acetamide

The methodologies described in Example 30D substituting 2-(4-chloro-3-fluorophenoxy)acetic acid for 3-(2-(4-chloro-3-fluorophenoxy)acetamido)bicyclo[1.1.1]pentane-1-carboxylic acid and substituting Example 21B for Example 30C gave the title compound. ¹H NMR (400 MHz, DMSO-d₆) δ ppm 7.99 (dd, J=7.9, 5.2 Hz, 1H), 7.49 (t, J=8.9 Hz, 1H), 7.36 (d, J=2.2 Hz, 1H), 7.34 (d, J=2.2 Hz, 1H), 7.06 (dd, J=11.4, 2.8 Hz, 1H), 6.92-6.85 (m, 2H), 6.88-6.80 (m, 1H), 5.03 (dtt, J=8.5, 6.2, 2.8 Hz, 1H), 4.75-4.65 (m, 1H), 4.52 (s, 2H), 4.32 (ddt, J=10.4, 6.4, 1.7 Hz, 1H), 4.22-4.13 (m, 1H), 3.92-3.85 (m, 1H), 3.81 (dddd, J=10.1, 8.1, 4.3, 1.5 Hz, 2H), 3.75 (m, 1H), 3.12 (td, J=10.3, 1.4 Hz, 1H), 1.92-1.78 (m, 2H), 1.66-1.46 (m, 2H); MS (APCI⁺) m/z 497 (M+H)⁺.

### Example 22: (2R,4R)-6-chloro-4-hydroxy-N-[(3R,6S)-6-({[4-(trifluoromethyl)phenyl]methyl}carbamoyl)oxan-3-yl]-3,4-dihydro-2H-1-benzopyran-2-carboxamide and (2S,4S)-6-chloro-4-hydroxy-N-[(3R,6S)-6-({[4-(trifluoromethyl)phenyl]methyl}carbamoyl)oxan-3-yl]-3,4-dihydro-2H-1-benzopyran-2-carboxamide (Compound 121)

The methodologies described in Example 5 substituting Example 38 for Example 4 and purifying by preparative HPLC (Phenomenex® Luna® C18(2) 10 μm 100 Å AXIA™ column (250 mm 50 mm) using a 30-100% gradient of acetonitrile (A) and 0.1% trifluoroacetic acid in water (B) over 25 minutes, at a flow rate of 50 mL/minute) gave the title compounds. ¹H NMR (400 MHz, DMSO-d₆, dr 20:1) δ ppm 8.41 (td, J=6.3, 1.7 Hz, 1H), 8.04 (t, J=8.3 Hz, 0.03H), 7.96 (dd, J=8.1, 2.3 Hz, 1H), 7.90 (d, J=8.4 Hz, 0.03H), 7.68 (d, J=8.1 Hz, 2H), 7.46 (d, J=8.0 Hz, 2H), 7.39 (dd, J=2.7, 0.9 Hz, 1H), 7.33-7.22 (m, 0.08H), 7.22-7.16 (m, 1H), 6.98 (dd, J=43.5, 8.9 Hz, 0.05H), 6.88 (d, J=8.7 Hz, 1H), 6.12 (s, 0.01H), 4.81 (dd, J=10.6, 5.9 Hz, 1H), 4.64 (dd, J=11.8, 2.3 Hz, 1H), 4.59 (t, J=3.6 Hz, 0.05H), 4.35 (d, J=6.3 Hz, 2H), 3.92 (dddd, J=8.0, 6.3, 4.6, 1.9 Hz, 1H), 3.88-3.73 (m, 2H), 3.30-3.18 (m, 1H), 2.35 (ddt, J=13.0, 5.7, 2.5 Hz, 1H), 2.08-1.99 (m, 1H), 1.96-1.89 (m, 1H), 1.80-1.68 (m, 1H), 1.71-1.58 (m, 1H), 1.55-1.40 (m, 1H); MS (APCI⁺) m/z 513 (M+H)⁺.

### Example 23: (2R,4R)-6-chloro-N-(3R,6S)-6-[3-(4-chlorophenoxy)azetidine-1-carbonyl]oxan-3-yl)-4-hydroxy-3,4-dihydro-2H-1-benzopyran-2-carboxamide and (2S,4S)-6-chloro-N-{(3R,6S)-6-[3-(4-chlorophenoxy)azetidine-1-carbonyl]oxan-3-yl}-4-hydroxy-3,4-dihydro-2H-1-benzopyran-2-carboxamide (Compound 122)

The methodologies described in Example 5 substituting Example 40 for Example 4 and purifying by preparative HPLC (Phenomenex® Luna® C18(2) 10 μm 100 Å AXIA™ column (250 mm×50 mm) using a 30-100% gradient of acetonitrile (A) and 0.1% trifluoroacetic acid in water (B) over 25 minutes, at a flow rate of 50 mL/minute) gave the title compounds. ¹H NMR (400 MHz, DMSO-d₆) δ ppm 7.92 (t, J=7.0 Hz, 1H), 7.41-7.29 (m, 3H), 7.20 (dd, J=8.7, 2.7 Hz, 1H), 6.96-6.84 (m, 3H), 5.03 (dq, J=6.6, 3.3 Hz, 1H), 4.81 (dd, J=10.6, 5.9 Hz, 1H), 4.76-4.65 (m, 1H), 4.64 (dd, J=11.8, 2.3 Hz, 1H), 4.37-4.27 (m, 1H), 4.18 (dt, J=10.2, 3.7 Hz, 1H), 3.92-3.71 (m, 3H), 3.23-3.11 (m, 1H), 2.39-2.29 (m, 1H), 1.90 (s, 1H), 1.86 (dt, J=9.2, 2.8 Hz, 1H), 1.72 (dtd, J=12.6, 11.0, 2.7 Hz, 1H), 1.69-1.52 (m, 2H); MS (APCI⁺) m/z 521 (M+H)⁺.

### Example 24: rac-(2R,4R)-6-chloro-4-hydroxy-N-[3-(([5-(trifluoromethyl)pyridin-2-yl]methyl)carbamoyl)bicyclo[1.1.1]pentan-1-yl]-3,4-dihydro-2H-1-benzopyran-2-carboxamide (Compound 123)

The reaction and purification conditions described in Example 6C substituting the product of Example 35 for the product of Example 6B gave the title compound. ¹H NMR (400 MHz, DMSO-d₆) δ ppm 8.92-8.87 (m, 1H), 8.70 (s, 1H), 8.54 (t, J=6.0 Hz, 1H), 8.19 (dd, J=8.3, 2.4 Hz, 1H), 7.45 (d, J=8.3 Hz, 1H), 7.38 (dd, J=2.7, 0.9 Hz, 1H), 7.20 (ddd, J=8.8, 2.7, 0.7 Hz, 1H), 6.88 (d, J=8.7 Hz, 1H), 5.70 (s, 1H), 4.85-4.76 (m, 1H), 4.60 (dd, J=12.0, 2.3 Hz, 1H), 4.43 (d, J=6.0 Hz, 2H), 2.36 (ddd, J=12.9, 5.9, 2.3 Hz, 1H), 2.25 (s, 6H), 1.77-1.63 (m, 1H); MS (ESI⁺) m/z 496 (M+H)⁺.

### Example 25: 2-(4-chloro-3-fluorophenoxy)-N-(2-hydroxy-4-{5-[(1s,3s)-3-(trifluoromethoxy)cyclobutyl]-1,3,4-oxadiazol-2-yl}bicyclo[2.2.2]octan-1-yl)acetamide (Compound 124)

### Example 25A: Dimethyl 2-oxobicyclo[2.2.2]octane-1,4-dicarboxylate

To a mixture of dimethyl bicyclo[2.2.2]octane-1,4-dicarboxylate (3.89 g, 17.19 mmol, Enamine) in acetic acid (40 mL) was added chromium trioxide (3.44 g, 34.4 mmol) at 20° C., and then the mixture was stirred at 90° C. for 18 hours. The reaction mixture was diluted with ethyl acetate (200 mL), poured into water (100 mL) and adjusted to pH=9 with solid NaHCO₃. The aqueous layer was extracted with ethyl acetate (3×200 mL). The organic phase was washed with brine (300 mL), dried over Na₂SO₄ and concentrated under reduced pressure. The residue was purified by column chromatography on silica gel (petroleum ether:ethyl acetate=20:1-10:1) to give crude title compound which was treated with petroleum ether (50 mL). The solid was collected by filtration and dried under high vacuum to give 0.8 g of the title compound. ¹H NMR (400 MHz, DMSO-d₆), δ ppm 1.68-2.16 (m, 8H), 2.25-2.35 (m, 2H), 2.58 (s, 2H), 3.64 (s, 1H), 3.70 (s, 3H), 3.74 (s, 3H).

### Example 25B: 4-(methoxycarbonyl)-2-oxobicyclo[2.2.2]octane-1-carboxylic Acid

To a solution of Example 25A (8.4 g, 33.2 mmol) in tetrahydrofuran (80 mL) and methanol (20 mL) was added a solution of lithium hydroxide monohydrate (1.116 g, 26.6 mmol) in water (20 mL) at 0° C., and the resulting mixture was stirred for 48 hours at 25° C. The mixture was concentrated under reduced pressure at 25° C., and the residue was diluted with water (40 mL) and extracted with 2-methoxy-2-methylpropane (2×80 mL). The aqueous layer was adjusted to pH=2 with aqueous 0.5 N HCl, and the precipitate was collected by filtration and dried under high vacuum to give the title compound (4 g, yield 50.6%). ¹H NMR (400 MHz, CDCl₃) δ ppm 1.88-2.12 (m, 7H), 2.27-2.39 (m, 2H), 2.60 (s, 2H), 3.72 (s, 1H), 3.75 (s, 3H).

### Example 25C: 4-tert-butyl 1-methyl 2-oxobicyclo[2.2.2]octane-1,4-dicarboxylate

To a solution of Example 25B (4 g, 16.80 mmol) in t-butanol (60 mL) was added pyridine (9.57 g, 121 mmol) and N,N-dimethylpyridin-4-amine (2.052 g, 16.80 mmol). Then di-tert-butyl dicarbonate (18.33 g, 84 mmol) was added slowly at 20° C., and the mixture was stirred at 35° C. for 24 hours. The resulting solution was concentrated under reduced pressure, and the residue was partitioned between ethyl acetate (100 mL) and water (100 mL). The organic phase was washed with water (2×100 mL), dried with Na₂SO₄ and concentrated under reduced pressure to give the title compound (5.5 g) which was used in the subsequent step without further purification. ¹H NMR (400 MHz, CDCl₃) δ ppm 1.37 (s, 9H), 1.79 (br d, J=12.35 Hz, 2H), 1.83-2.00 (m, 4H), 2.21 (br d, J=13.33 Hz, 2H), 2.46 (s, 2H), 3.68 (s, 3H).

### Example 25D: 4-(tert-butoxycarbonyl)-2-oxobicyclo[2.2.2]octane-1-carboxylic Acid

To a solution of Example 25C (5.5 g, 19.48 mmol) in tetrahydrofuran (80 mL) and methanol (20 mL) was added a solution of NaOH (0.779 g, 19.48 mmol) in water (20 mL) at 0° C., and the mixture was stirred at 0° C. to 25° C. for 12 hours. The mixture was concentrated under reduced pressure at 25° C. The residue was diluted with water (30 mL) and washed with 2-methoxy-2-methylpropane (2×50 mL). The aqueous layer was acidified to pH=1 with aqueous 1 N HCl, and the precipitate was collected by filtration and dried under high vacuum to give the title compound (2.4 g, yield 41%). ¹H NMR (400 MHz, CDCl₃), δ ppm 1.22 (s, 1H), 1.41-1.53 (m, 9H), 1.78-1.98 (m, 2H), 2.03-2.27 (m, 6H), 2.57-2.69 (m, 2H).

### Example 25E: Tert-Butyl 4-(((benzyloxy)carbonyl)amino)-3-oxobicyclo[2.2.2]octane-1-carboxylate

To a solution of Example 25D (1 g, 3.73 mmol) in toluene (100 mL) was added triethylamine (1.558 mL, 11.18 mmol) and diphenyl phosphorazidate (2.051 g, 7.45 mmol) sequentially at 20° C., and the mixture was stirred for 2 hours at 120° C. under N₂. Then benzyl alcohol (1.163 mL, 11.18 mmol) was added at 120° C., and the mixture was stirred at 120° C. for 12 hours. The reaction mixture was cooled to 25° C. and concentrated under reduced pressure. The residue was diluted with water (50 mL) and extracted with ethyl acetate (2-100 mL). The organic phase was dried with Na₂SO₄ and concentrated under reduced pressure, and the residue was purified by column chromatography on silica gel eluted with petroleum ether and ethyl acetate (100:1 to 30:1 to 10:1) to give the title compound (0.95 g, yield 62.5%). ¹H NMR (400 MHz, CDCl₃) δ ppm 1.37 (s, 9H), 1.50-1.56 (m, 2H), 1.70-1.88 (m, 3H), 1.97-2.12 (m, 3H), 2.55 (s, 2H), 2.72-2.90 (m, 2H), 4.99 (s, 2H), 5.92 (br s, 1H), 7.25-7.31 (m, 5H).

### Example 25F: Tert-Butyl 4-amino-3-oxobicyclo[0.2.2]octane-1-carboxylate

To a mixture of Pd(OH)₂ (600 mg, 4.27 mmol) in tetrahydrofuran (60 mL) was added a solution of Example 25E (2 g, 4.82 mmol) in tetrahydrofuran (60 mL) at 20° C. under argon, and the resulting mixture was stirred for 2 hours under H₂ at 15 psi. The resulting mixture was filtered through a pad of diatomaceous earth, and the cake was washed with ethyl acetate (30 mL). Water (20 mL) was added, and the resulting mixture was adjusted to pH=1 with aqueous 1.2 M HCl. The two phases were separated, and the aqueous layer was washed with ethyl acetate (2×20 mL). The aqueous layer was lyophilized to give the title compound (1.2 g, yield 88%). ¹H NMR (400 MHz, CD30D) δ ppm 1.46-1.49 (m, 9H), 1.94-2.07 (m, 4H), 2.13-2.25 (m, 4H), 2.74 (s, 2H).

### Example 25G: Tert-Butyl 4-(2-(4-chloro-3-fluorophenoxy)acetamido)-3-oxobicyclo[2.2.2]octane-1-carboxylate

A mixture of Example 25F (0.51 g, 1.849 mmol), 2-(4-chloro-3-fluorophenoxy)acetic acid (0.435 g, 2.127 mmol) and N-ethyl-N-isopropylpropan-2-amine (0.969 mL, 5.55 mmol) in N,N-dimethylformamide (10.0 mL) was treated with 2-(3H-[1,2,3]triazolo[4,5-b]pyridin-3-yl)-1,1,3,3-tetramethylisouronium hexafluorophosphate(V) (0.703 g, 1.849 mmol), and the reaction mixture was stirred at ambient temperature overnight. Water (100 mL) was added dropwise, and stirring was continued for 15 minutes. The precipitate was collected by filtration, washed with water and heptane, and dried under vacuum to give 0.74 g of the title compound. ¹H NMR (400 MHz, DMSO-d₆) δ ppm 7.67 (s, 1H), 7.45 (t, J=8.9 Hz, 1H), 7.04 (dd, J=11.3, 2.9 Hz, 1H), 6.81 (ddd, J=9.0, 2.9, 1.2 Hz, 1H), 4.52 (s, 2H), 2.53 (d, J=1.3 Hz, 2H), 2.46-2.29 (m, 2H), 1.94 (t, J=9.9 Hz, 2H), 1.87-1.79 (m, 1H), 1.78 (d, J=10.5 Hz, 3H), 1.36 (s, 9H); MS (ESI⁺) m/z 426.1 (M+H)⁺.

### Example 25H: 4-(2-(4-chloro-3-fluorophenoxy)acetamido)-3-oxobicyclo[2.2.2]octane-1-carboxylic Acid

To a solution of Example 25G (0.73 g, 1.714 mmol) in dichloromethane (10.0 mL) was added 2,2,2-trifluoroacetic acid (1.321 mL, 17.14 mmol), and the reaction mixture was stirred at ambient temperature for 2 hours and 50° C. for 1 hour. Volatiles were removed under high vacuum. The residue was triturated with dichloromethane/heptane to give 0.63 g of the title compound. ¹H NMR (400 MHz, DMSO-d₆) δ ppm 12.53 (s, 1H), 7.71 (s, 1H), 7.49 (t, J=8.8 Hz, 1H), 7.08 (dd, J=11.4, 2.9 Hz, 1H), 6.85 (ddd, J=8.9, 2.9, 1.2 Hz, 1H), 4.57 (s, 2H), 2.59 (d, J=1.3 Hz, 2H), 2.42 (dd, J=11.5, 8.5 Hz, 2H), 2.09-1.93 (m, 2H), 1.84 (d, J=8.3 Hz, 4H); MS (ESI⁺) m/z 370.2 (M+H)⁺.

### Example 25O: Methyl 4-(2-(4-chloro-3-fluorophenoxy)acetamido)-3-oxobicyclo[2.2.2]octane-1-carboxylate

To a solution of Example 25H (4.5 g, 10.95 mmol) in methanol (100 mL) was added H₂SO₄ (5 mL, 92 mmol) at 20° C., and the reaction mixture was stirred for 12 hours at 80° C.

The mixture was concentrated under reduced pressure, and the residue was diluted with water (100 mL), and the mixture was extracted with ethyl acetate (2×200 mL). The organic phase was dried with Na₂SO₄ and concentrated under reduced pressure. The residue was treated with methanol, the solid was collected by filtration, and dried by high vacuum to give the title compound (2.66 g, yield 55.7%). ¹H NMR (400 MHz, DMSO-d₆) δ ppm 1.81-1.92 (m, 4H), 1.96-2.08 (m, 2H), 2.42 (br dd, J=11.19, 8.74 Hz, 2H), 2.64 (s, 2H), 3.63 (s, 4H), 4.58 (s, 2H), 6.86 (dt, J=8.93, 1.41 Hz, 1H), 7.09 (dd, J=11.43, 2.87 Hz, 1H), 7.50 (t, J=8.86 Hz, 1H), 7.73 (s, 1H).

### Example 25J: Methyl 4-(2-(4-chloro-3-fluorophenoxy)acetamido)-3-hydroxybicyclo[2.2.2]octane-1-carboxylate

To a solution of Example 25I (2 g, 4.69 mmol) in methanol (50 mL) was added NaBH₄ (0.124 g, 3.28 mmol) at 0° C. and the reaction mixture was stirred for 3 hours at the same temperature. The reaction was quenched with saturated NH₄Cl solution, and the resulting mixture was concentrated under reduced pressure. The residue was diluted with water (30 mL) and extracted with ethyl acetate (2×50 mL). The organic phase was dried with Na₂SO₄ and concentrated under reduced pressure to give the title compound (2.1 g, yield 89%) which was used in the next step directly. MS (ESI+) m/z 386.0 (M+H)⁺.

### Example 25K: Methyl 3-((tert-butyldimethylsilyl)oxy)-4-(2-(4-chloro-3-fluorophenoxy)acetamido)bicyclo[2.2.2]octane-1-carboxylate

To a solution of Example 25J (2 g, 4.15 mmol) in CH₂Cl₂ (50 mL) was added 2,6-dimethylpyridine (1.777 g, 16.59 mmol) and tert-butyldimethylsilyl trifluoromethanesulfonate (2.74 g, 10.37 mmol) in order at 0° C., and the reaction mixture was stirred for 3 hours at 0° C. Saturated aqueous NH₄Cl (100 mL) was added, the two phases were separated, and the organic phase was dried with Na₂SO₄ and concentrated under reduced pressure. The residue was purified by reverse phase MPLC (Stationary phase: SNAP C18 120 g, 25˜35 μm, 100 Å, Mobile phase: A: trifluoroacetic acid/H₂O=0.05% volume/volume; B: acetonitrile, flow rate: 50 mL/minute; gradient (the percent of B): 5%-10% 5 minutes; 10%-30% 10 minutes; 30%-40% 15 minutes 40%-100% 20 minutes; 100% 6 minutes), and the desired fractions were concentrated under reduced pressure. The residue was basified by adding water and 2 g of NaHCO₃. The mixture was extracted with ethyl acetate (2×100 mL). The organic phase was dried with Na₂SO₄ and concentrated under reduced pressure to give the title compound (2.8 g, yield 99%). ¹H NMR (400 MHz, CDCl₃) δ ppm 0.00 (s, 3H), 0.06 (s, 3H), 0.84 (s, 10H), 1.62-1.76 (m, 2H), 1.77-1.98 (m, 7H), 2.24 (br dd, J=13.14, 9.60 Hz, 1H), 2.34-2.45 (m, 1H), 3.62 (s, 3H), 4.04-4.13 (m, 1H), 4.29 (d, J=0.98 Hz, 2H), 6.41 (br s, 1H), 6.61 (br d, J=8.93 Hz, 1H), 6.68 (dd, J=10.39, 2.69 Hz, 1H), 7.20-7.33 (m, 1H).

### Example 25L: N-(2-((tert-butyldimethylsilyl)oxy)-4-(hydrazinocarbonyl)bicyclo[2.2.2]octan-1-yl)-2-(4-chloro-3-fluorophenoxy)acetamide

A mixture of Example 25K (1.0 g, 2.000 mmol) and hydrazine monohydrate (1.471 mL, 30.0 mmol) was stirred at 120° C. for 16 hours. The resulting solution was cooled to ambient temperature. Water was added, and the mixture was extracted with ethyl acetate.

The organic layer was washed with brine, dried over magnesium sulfate and filtered. The filtrate was concentrated, and the residue was purified by HPLC (Phenomenex® Luna® C18(2) 10 μm 100 Å AXIA™ column (250 mm×50 mm) using a 30-100% gradient of acetonitrile (A) and 0.1% trifluoroacetic acid in water (B) over 25 minutes, at a flow rate of 50 mL/minute) to give 110 mg of the title compound. ¹H NMR (400 MHz, DMSO-d₆) δ ppm 10.12 (s, 1H), 7.47 (d, J=8.9 Hz, 1H), 7.16-6.97 (m, 2H), 6.82-6.75 (m, 1H), 4.46-4.25 (m, 3H), 2.23-2.10 (m, 2H), 1.80-1.57 (m, 7H), 1.51 (dt, J=13.5, 2.4 Hz, 1H), 0.84 (s, 9H), 0.02 (s, 3H), −0.03 (s, 3H).

### Example 25M: (cis)-benzyl 3-hydroxycyclobutanecarboxylate

To a solution of benzyl 3-oxocyclobutanecarboxylate (5.0 g, 24.48 mmol) in methanol (50 mL), sodium tetrahydroborate (0.926 g, 24.48 mmol) was added portionwise at −30° C. over 10 minutes followed by stirring at the same temperature for 3 hours. The mixture was cooled with an ice bath, saturated ammonium chloride was added carefully, and volatiles were removed under vacuum. The residue was extracted with ethyl acetate. The combined organic layer was dried over magnesium sulfate and filtered. The filtrate was concentrated, and the residue was purified on silica gel (0˜60% ethyl acetate in heptane) to give 2.55 g of the title compound. ¹H NMR (400 MHz, DMSO-d₆) δ ppm 7.42 7.28 (m, 5H), 5.21 (d, J=7.0 Hz, 1H), 5.08 (s, 2H), 3.97 (tq, J=8.3, 6.9 Hz, 1H), 2.68-2.54 (m, 1H), 2.40 (dddt, J=10.2, 6.8, 5.2, 2.5 Hz, 2H), 2.04-1.90 (m, 2H).

### Example 25N: (cis)-benzyl 3-(trifluoromethoxy)cyclobutanecarboxylate

The title compound was synthesized using the same procedure as described in Example 13O substituting Example 13N with Example 25M. ¹H NMR (400 MHz, DMSO-d₆) δ ppm 7.43-7.29 (m, 4H), 5.11 (s, 2H), 4.77 (p, J=7.5 Hz, 1H), 2.94-2.81 (m, 1H), 2.63 (dtt, J=9.7, 7.2, 2.3 Hz, 2H), 2.40-2.26 (m, 2H).

### Example 25O: (cis)-3-(trifluoromethoxy)cyclobutanecarboxylic Acid

A mixture of Example 25N (0.1 g, 0.365 mmol) and sodium hydroxide (0.912 mL, 1.823 mmol) in tetrahydrofuran (0.7 mL) was stirred at ambient temperature overnight. Solvent was removed under vacuum, and the residue was partitioned between dichloromethane and 1 N HCl. The organic layer was dried over magnesium sulfate and concentrated to give 0.047 g of the title compound which was used without further purification. ¹H NMR (400 MHz, DMSO-d₆) δ ppm 12.40 (brs, 1H), 5.75 (s, 1H), 4.74 (p, J=7.4 Hz, 1H), 2.77 2.52 (m, 3H), 2.34 2.21 (m, 2H).

### Example 25P: N-(2-((tert-butyldimethylsilyl)oxy)-4-(2-((cis)-3-(trifluoromethoxy)cyclobutanecarbonyl)hydrazinecarbonyl)bicyclo[2.2.2]octan-1-yl)-2-(4-chloro-3-fluorophenoxy)acetamide

To a mixture of Example 25L, Example 250 (0.040 g, 0.220 mmol), and N-ethyl-N-isopropylpropan-2-amine (0.123 mL, 0.704 mmol) in N,N-dimethylformamide (2.5 mL), 2-(3H-[1,2,3]triazolo[4,5-b]pyridin-3-yl)-1,1,3,3-tetramethylisouronium hexafluorophosphate(V) (0.084 g, 0.220 mmol) was added, and the mixture was stirred at ambient temperature for 2 hours. Volatiles were removed under high vacuum, and the residue was purified by HPLC (Phenomenex® Luna® C18(2) 10 μm 100 Å AXIA™ column (250 mm-50 mm). A 30-100% gradient of acetonitrile (A) and 0.1% trifluoroacetic acid in water (B) was used over 25 minutes, at a flow rate of 50 mL/minute) to give 65 mg of the title compound. ¹H NMR (400 MHz, DMSO-d₆) δ ppm 9.62 (d, J=1.5 Hz, 1H), 9.30 (d, J=1.6 Hz, 1H), 7.46 (t, J=8.8 Hz, 1H), 7.10 (d, J=11.7 Hz, 1H), 7.01 (dd, J=11.4, 2.8 Hz, 1H), 6.79 (ddd, J=9.0, 2.9, 1.2 Hz, 1H), 4.75 (p, J=7.6 Hz, 1H), 4.46-4.31 (m, 3H), 2.63 (qd, J=9.5, 7.4 Hz, 1H), 2.51-2.40 (m, 2H), 2.29-2.07 (m, 4H), 1.84 (ddd, J=10.9, 8.3, 4.6 Hz, 1H), 1.80-1.68 (m, 3H), 1.64 (qd, J=12.8, 10.9, 5.5 Hz, 3H), 1.50 (dt, J=13.6, 2.4 Hz, 1H), 0.81 (s, 9H), 0.00 (s, 3H), −0.06 (s, 3H).

### Example 25Q: N-(2-((tert-butyldimethylsilyl)oxy)-4-(5-((cis)-3-(trifluoromethoxy)cyclobutyl)-1,3,4-oxadiazol-2-yl)bicyclo[2.2.2]octan-1-yl)-2-(4-chloro-3-fluorophenoxy)acetamide

To a suspension of Example 25P (0.065 g, 0.098 mmol) in acetonitrile (2.0 mL) was added N-ethyl-N-isopropylpropan-2-amine (0.051 mL, 0.293 mmol), followed by 4-methylbenzene-1-sulfonyl chloride (0.037 g, 0.195 mmol). The reaction mixture was stirred at ambient temperature overnight. Volatiles were removed, and the residue was partitioned between water and ethyl acetate. The organic layer was washed with brine, dried over magnesium sulfate and filtered. The filtrate was concentrated, and the residue was purified by HPLC (Phenomenex® Luna® C18(2) 10 μm 100 Å AXIA™ column (250 mm×50 mm). A 45-100% gradient of acetonitrile (A) and 0.1% trifluoroacetic acid in water (B) was used over 25 minutes, at a flow rate of 50 mL/minute) to give 44 mg of the title compound. ¹H NMR (400 MHz, DMSO-d₆) δ ppm 7.45 (t, J=8.9 Hz, 1H), 7.20 (s, 1H), 7.07-6.96 (m, 1H), 6.79 (ddd, J=8.9, 2.9, 1.2 Hz, 1H), 4.84 (p, J=7.4 Hz, 1H), 4.47 (ddd, J=9.4, 5.4, 2.9 Hz, 1H), 4.42-4.34 (m, 2H), 2.78 (dtt, J=9.6, 7.4, 2.6 Hz, 2H), 2.46-2.22 (m, 4H), 1.98-1.76 (m, 4H), 1.78-1.62 (m, 3H), 0.81 (s, 9H), 0.00 (s, 3H), −0.05 (s, 3H).

### Example 25R: 2-(4-chloro-3-fluorophenoxy)-N-(2-hydroxy-4-{5-[(1s,3s)-3-(trifluoromethoxy)cyclobutyl]-1,3,4-oxadiazol-2-yl}bicyclo[2.2.2]octan-1-yl)acetamide

A solution of Example 25N (0.043 g, 0.066 mmol) in tetrahydrofuran (1.0 mL) was treated with tetrabutylammonium fluoride (0.166 mL, 0.166 mmol), and the reaction mixture was stirred at ambient temperature for 3 hours. The mixture was concentrated, and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 23 mg of the title compound. ¹H NMR (400 MHz, DMSO-d₆) δ ppm 7.47 (t, J=8.9 Hz, 1H), 7.35 (s, 1H), 7.05 (dd, J=11.4, 2.8 Hz, 1H), 6.83 (ddd, J=9.0, 2.8, 1.2 Hz, 1H), 5.19 (s, 1H), 4.87 (p, J=7.5 Hz, 1H), 4.48 (s, 2H), 4.12 (dd, J=7.0, 4.3 Hz, 1H), 4.04-3.95 (m, 1H), 2.80 (dddt, J=9.7, 7.4, 5.2, 2.5 Hz, 2H), 2.48-2.40 (m, 1H), 2.35-2.25 (m, 1H), 2.14-2.02 (m, 2H), 2.02-1.76 (m, 5H), 1.76-1.55 (m, 2H); MS (APCI⁺) m/z 534.1 (M+H)⁺.

### Example 26: (2R,4R)-6-chloro-N-{(1R,3r,5S)-8-[3-(4-chlorophenoxy)propyl]-8-azabicyclo[3.2.1]octan-3-yl}-4-hydroxy-3,4-dihydro-2H-1-benzopyran-2-carboxamide (Compound 125)

The reaction and purification conditions described in Example 6C substituting the product of Example 2B for the product of Example 6B gave the title compound. ¹H NMR (400 MHz, DMSO-d₆) δ ppm 7.47 (d, J=6.3 Hz, 1H), 7.38 (dd, J=2.7, 0.9 Hz, 1H), 7.36-7.26 (m, 2H), 7.20 (dd, J=8.7, 2.7 Hz, 1H), 6.99-6.92 (m, 2H), 6.88 (d, J=8.7 Hz, 1H), 5.67 (d, J=6.1 Hz, 1H), 4.80 (dt, J=11.0, 5.8 Hz, 1H), 4.69 (dd, J=11.2, 2.6 Hz, 1H), 4.02 (t, J=6.4 Hz, 2H), 3.83 (q, J=6.6 Hz, 1H), 3.15-3.10 (m, 2H), 2.39 (t, J=6.9 Hz, 2H), 2.32 (ddd, J=12.9, 5.8, 2.7 Hz, 1H), 2.10-1.92 (m, 2H), 1.91-1.68 (m, 7H), 1.60 (dd, J=13.8, 8.1 Hz, 2H); MS (ESI⁺) m/z 505 (M+H)⁺.

### Example 27: rac-(2R,4R)-6-chloro-N-[(1r,4R)-4-{[(6-chloro-1H-benzimidazol-2-yl)methyl]carbamoyl}cyclohexyl]-4-hydroxy-3,4-dihydro-2H-1-benzopyran-2-carboxamide (Compound 126)

The reaction and purification conditions described in Example 6C substituting the product of Example 39B for the product of Example 6B gave the title compound. ¹H NMR (400 MHz, DMSO-d₆) δ ppm 8.38 (t, J=5.6 Hz, 1H), 7.88 (d, J=8.2 Hz, 1H), 7.52 (d, J=2.1 Hz, 1H), 7.48 (d, J=8.5 Hz, 1H), 7.41-7.36 (m, 2H), 7.20 (dd, J=8.7, 2.7 Hz, 1H), 7.12 (dd, J=8.5, 2.1 Hz, 1H), 6.89 (d, J=8.7 Hz, 1H), 5.72 (br s, 1H), 4.81 (dd, J=10.7, 6.0 Hz, 1H), 4.61 (dd, J=11.9, 2.2 Hz, 1H), 4.45 (d, J=5.5 Hz, 2H), 3.71-3.52 (m, 2H), 2.35 (ddd, J=13.1, 6.0, 2.4 Hz, 1H), 2.24-2.13 (m, 1H), 1.88-1.77 (m, 3H), 1.71 (q, J=12.0 Hz, 1H), 1.51-1.29 (m, 3H); MS (APCI⁺) m/z 517 (M+H)⁺.

### Example 28: (2R,4R)-6-chloro-N-(3-{[(5,6-difluoro-1H-benzimidazol-2-yl)methyl]carbamoyl}bicyclo[1.1.1]pentan-1-yl)-4-hydroxy-3,4-dihydro-2H-1-benzopyran-2-carboxamide (Compound 127)

The reaction and purification conditions described in Example 6C substituting the product of Example 36 for the product of Example 6B gave the title compound. ¹H NMR (500 MHz, DMSO-d₆) δ ppm 8.69 (s, 1H), 8.48 (t, J=5.8 Hz, 1H), 7.58-7.49 (m, 2H), 7.38 (dd, J=2.7, 1.0 Hz, 1H), 7.20 (dd, J=8.7, 2.7 Hz, 1H), 6.88 (d, J=8.7 Hz, 1H), 5.70 (br s, 1H), 4.80 (dd, J=10.7, 5.9 Hz, 1H), 4.59 (dd, J=12.0, 2.2 Hz, 1H), 4.43 (d, J=5.7 Hz, 2H), 2.35 (ddd, J=13.0, 5.9, 2.4 Hz, 1H), 2.24 (s, 6H), 2.07 (s, 1H), 1.69 (td, J=12.6, 10.8 Hz, 1H); MS (APCI⁺) m/z 503 (M+H)⁺.

### Example 29: (2R,4R)-6-chloro-4-hydroxy-N-(3-{[(1s,3S)-3-(trifluoromethoxy)cyclobutane-1-carbonyl]amino}bicyclo[1.1.1]pentan-1-yl)-3,4-dihydro-2H-1-benzopyran-2-carboxamide (Compound 128)

The reaction and purification conditions described in Example 6C substituting the product of Example 34 for the product of Example 6B gave the title compound. ¹H NMR (400 MHz, DMSO-d₆) δ ppm 8.66 (s, 1H), 8.52 (s, 1H), 7.37 (dd, J=2.7, 1.0 Hz, 1H), 7.20 (dd, J=8.7, 2.7 Hz, 1H), 6.88 (d, J=8.7 Hz, 1H), 5.69 (br s, 1H), 4.80 (dd, J=10.9, 6.0 Hz, 1H), 4.78-4.68 (m, 1H), 4.58 (dd, J=12.0, 2.3 Hz, 1H), 2.61-2.52 (m, 2H), 2.49-2.39 (m, 2H), 2.39-2.29 (m, 1H), 2.29-2.18 (m, 1H), 2.23 (s, 6H), 1.75-1.62 (m, 1H); MS (APCI⁺) m/z 456 (M−H₂O+H)⁺.

### Example 30: N-[(6-chloro-3,4-dihydro-2H-1-benzopyran-2-yl)methyl]-3-[2-(4-chloro-3-fluorophenoxy)acetamido]bicyclo[1.1.1]pentane-1-carboxamide (Compound 129)

### Example 30A: 6-chlorochroman-2-carbaldehyde

To a cooled (0° C.) solution of 6-chlorochroman-2-carboxylic acid (0.45 g, 2.1 mmol) in methanol (3.5 mL) was added thionyl chloride (0.39 mL, 5.3 mmol), and the mixture was then heated to 65° C. for 3 hours. The reaction mixture was then cooled to ambient temperature, concentrated, and diluted with saturated sodium bicarbonate solution. The aqueous layer was extracted with ethyl acetate (3×10 mL), and the combined organic layers were washed with water (10 mL) and brine (10 mL), dried (Na₂SO₄), and concentrated to provide methyl 6-chlorochroman-2-carboxylate.

To a cooled (−78° C.) suspension of methyl 6-chlorochroman-2-carboxylate (0.47 g, 2.1 mmol) in dichloromethane (0.77 mL) and toluene (3.1 mL) was added DIBAL-H (diisobutylaluminum hydride) (2.2 mL, 2.2 mmol, 1 M in toluene) dropwise. The reaction mixture stirred for 1.5 hours while remaining cold. This reaction mixture was then quenched with methanol (1 mL) and allowed to warm to ambient temperature. A saturated Rochelle salt aqueous solution (1 mL) was then added to the reaction which was stirred rapidly for 10 minutes. The reaction mixture was extracted with diethyl ether (3×5 mL), and the combined organic phases were concentrated under heated N₂ to provide the title compound as a mixture with remaining methyl 6-chlorochroman-2-carboxylate and (6-chlorochroman-2-yl)methanol. The residue was carried forward without further purification.

### Example 30B: N-benzyl-1-(6-chlorochroman-2-yl)methanamine

To a solution of the product of Example 30A (0.30 g, 1.5 mmol) in 2.4 weight % sodium acetate trihydrate and 3.6 weight % acetic acid in methanol (15 mL) was added benzylamine (0.17 mL, 1.5 mmol). To this reaction mixture was added sodium cyanoborohydride (0.24 g, 3.8 mmol) at ambient temperature, and the mixture was stirred for 2 hours, was concentrated, and purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give the title compound (0.18 g, 0.62 mmol, 41% yield). ¹H NMR (400 MHz, DMSO-d₆) δ ppm 9.33 (s, 2H), 7.58-7.48 (m, 2H), 7.51-7.37 (m, 3H), 7.21-7.11 (m, 2H), 6.84 (d, J=8.6 Hz, 1H), 4.38 (ddt, J=10.9, 8.7, 2.8 Hz, 1H), 4.32-4.20 (m, 2H), 3.27 (dd, J=13.2, 3.4 Hz, 1H), 3.19 (dd, J=13.2, 8.7 Hz, 1H), 2.79 (qdd, J=13.5, 8.4, 4.2 Hz, 2H), 2.03 (ddq, J=15.9, 5.9, 3.1, 2.6 Hz, 1H), 1.68 (dtd, J=13.6, 10.6, 5.9 Hz, 1H); MS (APCI⁺) m/z 288 (M+H)⁺.

### Example 30C: (6-chlorochroman-2-yl)methanamine

Example 30B (0.178 g, 0.621 mmol) in tetrahydrofuran (2.0 mL) was added to 10% Pd(OH)₂/C wet (0.0386 g, 0.115 mmol) in a 20 mL RS10 with a glass liner. 4M HCl in dioxane (0.50 mL, 2.0 mmol) was added. The reactor was purged with argon. The mixture was stirred at 1200 rpm under 55 psi of hydrogen at 25° C. After 20.4 hours, no reaction occurred, so ethanol (2.0 mL) and 10% Pd(OH)₂/C wet (0.208 g, 0.621 mmol) was added to the reaction mixture, and the solution was placed back under hydrogen pressure and allowed to stir for 4 days. Although there was incomplete conversion, some dehalogenation occurred, so then the mixture was filtered and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.028 g, 0.14 mmol, 23% yield). ¹H NMR (400 MHz, DMSO-d₆) δ ppm 8.00 (s, 3H), 7.25-7.17 (m, 1H), 7.14 (dd, J=8.7, 2.7 Hz, 1H), 6.81 (d, J=8.7 Hz, 1H), 4.22 (ddt, J=10.5, 8.2, 2.8 Hz, 1H), 3.18 (s, 1H), 3.12-3.04 (m, 1H), 2.80 (qdd, J=13.7, 8.5, 4.2 Hz, 2H), 2.09-1.98 (m, 1H), 1.68 (dtd, J=13.6, 10.7, 5.9 Hz, 1H).

### Example 30D: N-[(6-chloro-3,4-dihydro-2H-1-benzopyran-2-yl)methyl]-3-[2-(4-chloro-3-fluorophenoxy)acetamido]bicyclo[1.1.1]pentane-1-carboxamide

To a mixture of 3-(2-(4-chloro-3-fluorophenoxy)acetamido)bicyclo[1.1.1]pentane-1-carboxylic acid (0.050 g, 0.16 mmol, CALICO Life Sciences; AbbVie Inc.; Sidrauski, Carmela; et al. WO2017/193030, 2017, A1) and the product of Example 30C (0.033 g, 0.17 mmol) in N,N-dimethylformamide (0.91 mL) was added triethylamine (0.09 mL, 0.64 mmol) followed by 1-[bis(dimethylamino)methylene]-1H-1,2,3-triazolo[4,5-b]pyridinium 3-oxid hexafluorophosphate (HATU, 0.067 g, 0.18 mmol). This reaction mixture was allowed to stir at ambient temperature for 5 hours. Then the reaction mixture was diluted with water (0.5 mL) and filtered. The filtrate was purified by preparative HPLC (Phenomenex® Luna® C18(2) 10 μm 100 Å AXIA™ column (250 mm 50 mm). A 30-100% gradient of acetonitrile (A) and 0.1% trifluoroacetic acid in water (B) was used over 25 minutes, at a flow rate of 50 mL/minute) to give the title compound (0.028 g, 0.057 mmol, 36% yield). ¹H NMR (400 MHz, DMSO-d₆) δ ppm 8.71 (s, 1H), 8.00 (t, J=5.9 Hz, 1H), 7.50 (t, J=8.9 Hz, 1H), 7.11 (d, J=14.8 Hz, 1H), 7.07 (dd, J=5.5, 2.8 Hz, 1H), 6.85 (ddd, J=9.0, 2.9, 1.2 Hz, 1H), 6.75 (d, J=8.7 Hz, 1H), 4.47 (s, 2H), 4.05 (dtd, J=9.8, 6.0, 2.3 Hz, 1H), 3.45-3.33 (m, 1H), 3.26 (dt, J=13.6, 6.0 Hz, 1H), 2.80-2.68 (m, 2H), 2.20 (s, 6H), 2.02-1.89 (m, 1H), 1.56 (dtd, J=13.6, 9.8, 6.6 Hz, 1H); MS (APCI⁺) m/z 493 (M+H)⁺.

### Example 31: 6-chloro-N-{(1r,4r)-4-[2-(4-chloro-3-fluorophenoxy)acetamido]cyclohexyl}-4-oxo-3,4-dihydro-2H-1-benzopyran-2-carboxamide (Compound 130)

### Example 31A: Tert-Butyl ((1r,4r)-4-(2-(4-chloro-3-fluorophenoxy)acetamido)cyclohexyl)carbamate

To a solution of 2-(4-chloro-3-fluorophenoxy)acetic acid (15 g, 69 mmol) and 1-[bis(dimethylamino)methylene]-1H-1,2,3-triazolo[4,5-b]pyridinium 3-oxid hexafluorophosphate (HATU, 39.5 g, 104 mmol) in tetrahydrofuran (600 mL) was added N-ethyl-N-isopropylpropan-2-amine (24.2 mL, 138 mmol). Then the mixture was stirred at 15° C. for 15 minutes, followed by the addition of tert-butyl ((1r,4r)-4-aminocyclohexyl)carbamate (14.8 g, 69.2 mmol). The reaction mixture was stirred at 15° C. for 12 hours, was filtered, and the filter cake was washed with tetrahydrofuran (10 mL) to give the title compound (26.0 g, 64.7 mmol, 93% yield). ¹H NMR (400 MHz, DMSO-d₆) δ ppm 7.91 (d, J=7.6 Hz, 1H), 7.46 (t, J=8.80 Hz, 1H), 7.04 (d, J=8.20 Hz, 1H), 6.82 (d, J=10.4 Hz, 1H), 6.67 (d, J=7.6 Hz, 1H), 4.45 (s, 2H), 3.51 (s, 1H), 3.15 (s, 1H), 1.69-1.76 (m, 4H), 1.15-1.34 (m, 14H).

### Example 31B: N-((1r,4r)-4-aminocyclohexyl)-2-(4-chloro-3-fluorophenoxy)acetamide, Hydrochloric Acid

To a solution of Example 31A (25.9 g, 64.3 mmol) in methanol (250 mL) was added a solution of hydrogen chloride (250 mL, 4 M in methanol) dropwise at 0° C., and the resulting mixture was allowed to warm to ambient temperature for 12 hours. Then methyl tert-butyl ether (1 L) was added, the mixture was cooled to 0° C., and a precipitate was generated. The resulting mixture stirred for 1 hour. The precipitate was collected by filtration was filtered and dried under high vacuum to give the title compound. ¹H NMR (400 MHz, D₂O) δ ppm 7.28 (t, J=8.80 Hz, 1H), 6.74-6.77 (m, 1H), 6.63-6.66 (m, 1H), 4.34 (s, 2H), 3.57-3.62 (m, 1H), 3.03-3.09 (m, 1H), 1.94 (d, J=12.4 Hz, 2H), 1.82 (d, J=12.0 Hz, 2H), 1.37-1.44 (m, 2H), 1.25-1.32 (m, 2H).

### Example 31C: 6-chloro-N-{(1r,4r)-4-[2-(4-chloro-3-fluorophenoxy)acetamido]cyclohexyl}-4-oxo-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 31B for Example 30C gave the title compound. ¹H NMR (501 MHz, DMSO-d₆) δ ppm 8.16 (d, J=8.0 Hz, 1H), 7.95 (d, J=8.1 Hz, 1H), 7.72-7.57 (m, 2H), 7.48 (t, J=8.9 Hz, 1H), 7.16 (d, J=8.7 Hz, 1H), 7.05 (dd, J=11.4, 2.9 Hz, 1H), 6.83 (ddd, J=9.0, 3.0, 1.1 Hz, 1H), 5.11 (dd, J=8.2, 5.2 Hz, 1H), 4.48 (s, 2H), 3.54 (d, J=33.1 Hz, 2H), 3.03-2.82 (m, 2H), 1.73 (d, J=37.6 Hz, 4H), 1.31 (q, J=12.3, 11.0 Hz, 4H); MS (APCI⁺) m/z 509 (M+H)⁺.

### Example 32: (2S,4S)-6-chloro-N-[(3R,6S)-6-{[(7-chloroimidazo[1,2-a]pyridin-2-yl)methyl]carbamoyl}oxan-3-yl]-4-hydroxy-3,4-dihydro-2H-1-benzopyran-2-carboxamide and (2R,4R)-6-chloro-N-[(3R,6S)-6-{[(7-chloroimidazo[1,2-a]pyridin-2-yl)methyl]carbamoyl}oxan-3-yl]-4-hydroxy-3,4-dihydro-2H-1-benzopyran-2-carboxamide (Compound 131)

The methodologies described in Example 5 substituting Example 41 for Example 4 and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compounds. ¹H NMR (501 MHz, DMSO-d₆, dr 5.6:1) δ ppm 8.78 (s, 1H), 8.77 (s, 1H), 8.42-8.36 (m, 1H), 8.03 (d, J=4.1 Hz, 1H), 8.01-7.96 (m, 2H), 7.92 (dd, J=8.1, 3.8 Hz, 0.18H), 7.50 (t, J=2.1 Hz, 0.18H), 7.44-7.36 (m, 3H), 7.24-7.17 (m, 1H), 7.07-7.01 (m, 0.18H), 6.93 (d, 0.1=8.8 Hz, 0.18H), 6.88 (d, J=8.7 Hz, 1H), 6.11 (t, J=5.4 Hz, 0.18H), 4.91 (t, J=5.4 Hz, 0.18H), 4.81 (dd, J=10.7, 5.9 Hz, 1H), 4.65 (dd, J=11.9, 2.3 Hz, 1H), 4.59 (d, J=3.1 Hz, 0.18H), 4.48 (d, J=6.0 Hz, 3H), 3.97-3.89 (m, 1H), 3.81 (dt, J=11.5, 2.5 Hz, 2H), 3.24 (dt, J=11.9, 10.6 Hz, 1H), 2.39-2.31 (m, 1H), 2.08-2.01 (m, 1H), 1.93 (s, 1H), 1.79-1.61 (m, 2H), 1.56-1.45 (m, 1H); MS (APCI⁺) m/z 520 (M+H)⁺.

### Example 33: (2R)-6-chloro-4-oxo-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide (Compound 132)

### Example 33A: 3-amino-N-((5-(trifluoromethyl)pyridin-2-yl)methyl)bicyclo[1.1.1]pentane-1-carboxamide, 2 Trifluoroacetic Acid

The reaction and purification conditions described in Examples 14A through 14B substituting 3-((tert-butoxycarbonyl)amino)bicyclo[1.1.1]pentane-1-carboxylic acid (Enamine) for 6-chloro-4-oxochroman-2-carboxylic acid, and (5-(trifluoromethyl)pyridin-2-yl)methanamine hydrochloride (Apollo) for tert-butyl (3-aminobicyclo[1.1.1]pentan-1-yl)carbamate (PharmaBlock) gave the title compound. MS (ESI⁺) m/z 286 (M+H)⁺.

### Example 33B: (2R)-6-chloro-4-oxo-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide

The reaction and purification conditions described in Example 2B substituting the product of Example 33A for the product of Example 2A gave the title compound. ¹H NMR (500 MHz, DMSO-d₆) δ ppm 8.95 (s, 1H), 8.91-8.86 (m, 1H), 8.53 (t, J=6.1 Hz, 1H), 8.18 (dd, J=8.2, 2.4 Hz, 1H), 7.68-7.61 (m, 2H), 7.44 (d, J=8.2 Hz, 1H), 7.21-7.13 (m, 1H), 5.09 (dd, J=8.3, 6.0 Hz, 1H), 4.42 (d, J=6.0 Hz, 2H), 3.03-2.88 (m, 2H), 2.23 (s, 6H); MS (APCI⁺) m/z 494 (M+H)⁺.

### Example 34: (2R)-6-chloro-4-oxo-N-(3-{[(1s,3S)-3-(trifluoromethoxy)cyclobutane-1-carbonyl]amino}bicyclo[1.1.1]pentan-1-yl)-3,4-dihydro-2H-1-benzopyran-2-carboxamide (Compound 133)

The reaction and purification conditions described in Examples 14A through 14C substituting the product of Example 250 for the product of Example 13P, and the product of Example 1B for 6-chloro-4-oxochroman-2-carboxylic acid gave the title compound. ¹H NMR (400 MHz, DMSO-d₆) δ ppm 8.92 (s, 1H), 8.52 (s, 1H), 7.67-7.59 (m, 2H), 7.20-7.12 (m, 1H), 5.07 (t, J=7.1 Hz, 1H), 4.73 (p, J=7.5 Hz, 1H), 2.94 (d, J=7.1 Hz, 2H), 2.57-2.52 (m, 1H), 2.48-2.37 (m, 2H), 2.28-2.20 (m, 2H), 2.20 (s, 6H); MS (APCI⁺) m/z 473 (M+H)⁺.

### Example 35: 6-chloro-4-oxo-N-[3-({[5-(trifluoromethyl)pyridin-2-yl]methyl}carbamoyl)bicyclo[1.1.1]pentan-1-yl]-3,4-dihydro-2H-1-benzopyran-2-carboxamide (Compound 134)

The reaction and purification conditions described in Example 33B substituting the racemic 6-chloro-4-oxochroman-2-carboxylic acid for (R)-6-chloro-4-oxochroman-2-carboxylic acid gave the title compound. ¹H NMR (501 MHz, DMSO-d₆) δ ppm 8.96 (s, 1H), 8.90-8.87 (m, 1H), 8.54 (t, J=6.0 Hz, 1H), 8.18 (dd, J=8.4, 2.4 Hz, 1H), 7.68-7.61 (m, 2H), 7.44 (d, J=8.2 Hz, 1H), 7.17 (dd, J=8.5, 0.7 Hz, 1H), 5.08 (dd, J=8.4, 5.9 Hz, 1H), 4.42 (d, J=6.0 Hz, 2H), 2.96 (d, J=3.6 Hz, 1H), 2.94 (s, 1H), 2.23 (s, 6H); MS (ESI⁺*) m/z 494 (M+H)⁺.

### Example 36: (2R)-6-chloro-N-(3-{[(5,6-difluoro-1H-benzimidazol-2-yl)methyl]carbamoyl}bicyclo[1.1.1]pentan-1-yl)-4-oxo-3,4-dihydro-2H-1-benzopyran-2-carboxamide (Compound 135)

The title compound was prepared using the methodologies described above. ¹H NMR (400 MHz, DMSO-d₆) δ ppm 12.34 (s, 1H), 8.94 (s, 1H), 8.48 (t, J=5.8 Hz, 1H), 7.68-7.60 (m, 2H), 7.54 (br s, 2H), 7.21-7.13 (m, 1H), 5.08 (dd, J=8.2, 6.2 Hz, 1H), 4.43 (d, J=5.8 Hz, 2H), 3.01-2.91 (m, 2H), 2.22 (s, 6H); MS (ESI⁺) m/z 501 (M+H)⁺.

### Example 37: rac-(2R,4R)-6-chloro-N-[(1r,4R)-4-{3-[5-(difluoromethyl)pyrazin-2-yl]-2-oxoimidazolidin-1-yl}cyclohexyl]-4-hydroxy-3,4-dihydro-2H-1-benzopyran-2-carboxamide (Compound 136)

### Example 37A: Tert-Butyl (2-(((1r,4r)-4-(((benzyloxy)carbonyl)amino)cyclohexyl)amino)ethyl)carbamate

To a mixture of benzyl ((1r,4r)-4-aminocyclohexyl)carbamate (2.5 g, 10.1 mmol) and tert-butyl (2-oxoethyl)carbamate (2.48 g, 15.6 mmol) in methanol (67 mL) stirred at ambient temperature was added acetic acid (4 mL) followed by sodium cyanoborohydride (1.39 g, 22.2 mmol) and trifluoroacetic acid (0.776 mL). After 18 hours, the resulting solution was concentrated under reduced pressure to less than 20 mL, filtered through a glass microfiber frit and directly purified by preparative HPLC [YMC TriArt™ C-18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (1.0 g, 2.55 mmol, 25% yield). MS (APCI⁺) m/z 392 (M+H)⁺.

### Example 37B: Benzyl ((1r,4r)-4-((2-aminoethyl)amino)cyclohexyl)carbamate

Trifluoroacetic acid (1 mL) was added to a dichloromethane (1.0 mL) solution of the product of Example 37A (1 g, 2.55 mmol) stirred at 0° C. The reaction mixture was slowly warmed up to ambient temperature over 30 minutes and then concentrated under reduced pressure. The residue was partitioned between dichloromethane (2×50 mL) and aqueous NaOH (2.5 M, 20 mL). The organic layers were combined and concentrated under reduced pressure. The resulting residue was taken up in methanol (˜20 mL) and filtered through a glass microfiber frit. The filtrate was concentrated under reduced pressure to give the title compound (0.72 g, 2.47 mmol, 97% yield). MS (ESI⁺) m/z 292 (M+H)⁺.

### Example 37C: Benzyl ((1r,4r)-4-(2-oxoimidazolidin-1-yl)cyclohexyl)carbamate

To a mixture of the product of Example 37B (0.715 g, 2.45 mmol) and 1,8-diazabicyclo[5.4.0]undec-7-ene (DBU, 0.055 mL, 0.368 mmol) in tetrahydrofuran (24 mL) was added N,N-carbonyldiimidazole (458 mg, 2.82 mmol). The resulting mixture was stirred at ambient temperature for 18 hours and then concentrated under reduced pressure. The residue was directly purified by preparative HPLC [YMC TriArt™ C18 Hybrid 5 μm column, 50×100 mm, flow rate 140 mL/minute, 0-100% gradient of acetonitrile in buffer (0.025 M aqueous ammonium bicarbonate, adjusted to pH 10 with ammonium hydroxide)] to give the title compound (267 mg, 0.84 mmol, 34% yield). MS (ESI⁺) m/z 318 (M+H)⁺.

### Example 37D: Benzyl ((1r,4r)-4-(3-(5-(difluoromethyl)pyrazin-2-yl)-2-oxoimidazolidin-1-yl)cyclohexyl)carbamate

2-Bromo-5-(difluoromethyl)pyrazine (Matrix, 44.5 mg, 0.213 mmol), 2-(dicyclohexylphosphino)-2′,4′,6′-triisopropylbiphenyl (XPhos, 11.7 mg, 0.025 mmol) tris(dibenzylideneacetone)dipalladium(0) (11.3 mg, 0.012 mmol), the product of Example 37C (52 mg, 0.164 mmol) and cesium carbonate (160 mg, 0.492 mmol) were added to a sealed tubed followed by dioxane (2 mL). The tube was degassed three times with a nitrogen back flush each time and then sealed. The reaction mixture was warmed to 55° C. and stirred for 3 hours and then at 100° C. for 2 hours. The mixture was cooled to ambient temperature and partitioned between dichloromethane (2×25 mL) and aqueous sodium carbonate (1.0 M, 20 mL). The organic layers were combined and dried over anhydrous sodium sulfate and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (65 mg, 0.146 mmol, 89% yield). ¹H NMR (400 MHz, DMSO-d₆) δ ppm 9.52 (d, J=1.5 Hz, 1H), 8.62-8.59 (m, 1H), 7.40-7.28 (m, 5H), 7.22 (d, J=7.8 Hz, 1H), 7.03 (t, J=54.6 Hz, 1H), 5.01 (s, 2H), 3.93 (dd, J=9.0, 6.9 Hz, 2H), 3.64 (ddt, J=11.8, 7.7, 4.0 Hz, 1H), 3.52 (t, J=8.0 Hz, 2H), 3.33-3.24 (m, 1H), 1.95-1.85 (m, 2H), 1.74-1.52 (m, 4H), 1.31 (qd, J=12.8, 3.8 Hz, 2H); MS (ESI⁺) m/z 446 (M+H)⁺.

### Example 37E: 1-((1r,4r)-4-aminocyclohexyl)-3-(5-(difluoromethyl)pyrazin-2-yl)imidazolidin-2-one

The product of Example 37D (60 mg, 0.135 mmol) was combined with trifluoroacetic acid (3 mL) in a sealed tube and stirred at 70° C. for 1 hour. The reaction was cooled to ambient temperature and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (34 mg, 0.11 mmol, 81% yield). MS (APCI⁺) m/z 312 (M+H)⁺.

### Example 37F: 6-chloro-N-((1r,4r)-4-(3-(5-(difluoromethyl)pyrazin-2-yl)-2-oxoimidazolidin-1-yl)cyclohexyl)-4-oxochroman-2-carboxamide

The reaction and purification conditions described in Example 2B substituting the product of Example 37E for the product of Example 2A, and 6-chloro-4-oxochroman-2-carboxylic acid (Princeton Bio) for the product of Example 1B gave the title compound. MS (APCI⁺) m/z 520 (M+H)⁺.

### Example 37G: rac-(2R,4R)-6-chloro-N-[(1r,4R)-4-{3-[5-(difluoromethyl)pyrazin-2-yl]-2-oxoimidazolidin-1-yl}cyclohexyl]-4-hydroxy-3,4-dihydro-2H-1-benzopyran-2-carboxamide

The reaction and purification conditions described in Example 6C substituting the product of Example 37F for the product of Example 6B gave the title compound. ¹H NMR (400 MHz, DMSO-d₆) δ ppm 9.64 (d, J=1.5 Hz, 1H), 8.51 (s, 1H), 7.45 (dd, J=2.6, 0.8 Hz, 1H), 7.19 (dd, J=8.7, 2.6 Hz, 1H), 6.86 (d, J=8.7 Hz, 1H), 6.68 (t, J=55.2 Hz, 1H), 6.40 (d, J=8.3 Hz, 1H), 4.91 (q, J=7.0 Hz, 1H), 4.65 (dd, J=9.2, 3.2 Hz, 1H), 4.06-4.00 (m, 2H), 3.91 (tt, J=12.1, 3.9 Hz, 1H), 3.80 (dtd, J=11.9, 8.0, 4.1 Hz, 1H), 3.57-3.50 (m, 2H), 2.68 (ddd, J=13.7, 5.7, 3.3 Hz, 1H), 2.26 (d, J=7.1 Hz, 1H), 2.21-2.12 (m, 2H), 2.08-1.98 (m, 1H), 1.96-1.83 (m, 2H), 1.70-1.57 (m, 2H), 1.48-1.27 (m, 2H); MS (APCI⁺) m/z 522 (M+H)⁺.

### Example 38: 6-chloro-4-oxo-N-[(3R,6S)-6-({[4-(trifluoromethyl)phenyl]methyl}carbamoyl)oxan-3-yl]-3,4-dihydro-2H-1-benzopyran-2-carboxamide (Compound 137)

### Example 38A: Tert-Butyl ((3R,6S)-6-((4-(trifluoromethyl)benzyl)carbamoyl)tetrahydro-2H-pyran-3-yl)carbamate

The methodologies described in Example 30D substituting (2S,5R)-5-((tert-butoxycarbonyl)amino)tetrahydro-2H-pyran-2-carboxylic acid (purchased from Astatech) for 3-(2-(4-chloro-3-fluorophenoxy)acetamido)bicyclo[1.1.1]pentane-1-carboxylic acid and substituting (4-(trifluoromethyl)phenyl)methanamine hydrochloride for Example 30C gave the title compound. MS (APCI⁺) m/z 303 (M−C(O)OC(CH₃)₃+H)⁺.

### Example 38B: (2S,5R)-5-amino-N-(4-(trifluoromethyl)benzyl)tetrahydro-2H-pyran-2-carboxamide

The methodologies described in 21B substituting Example 38A for Example 21A gave the title compound. MS (APCI⁺) m/z 303 (M+H)⁺.

### Example 38C: 6-chloro-4-oxo-N-[(3R,6S)-6-({[4-(trifluoromethyl)phenyl]methyl}carbamoyl)oxan-3-yl]-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 38B for Example 30C gave the title compound. ¹H NMR (500 MHz, DMSO-d₆) δ ppm 8.41 (td, J=6.3, 4.0 Hz, 1H), 8.25 (dd, J=7.9, 3.1 Hz, 1H), 7.70-7.61 (m, 4H), 7.45 (d, 0.1=8.1 Hz, 2H), 7.17 (dt, J=8.6, 0.8 Hz, 1H), 5.14 (td, J=6.6, 1.6 Hz, 1H), 4.34 (d, J=6.4 Hz, 2H), 3.94-3.80 (m, 1H), 3.82-3.78 (m, 1H), 3.78-3.68 (m, 1H), 3.17 (dt, J=25.9, 10.6 Hz, 1H), 3.00-2.95 (m, 2H), 2.02 (ddd, J=13.0, 8.2, 3.0 Hz, 1H), 1.89 (dd, J=43.0, 12.6 Hz, 1H), 1.60 (pd, J=12.8, 3.9 Hz, 1H), 1.47 (tdd, J=11.4, 6.4, 3.8 Hz, 1H); MS (APCI⁺) m/z 511 (M+H)⁺.

### Example 39: 6-chloro-N-[(1r,4r)-4-{[(6-chloro-1H-benzimidazol-2-yl)methyl]carbamoyl}cyclohexyl]-4-oxo-3,4-dihydro-2H-1-benzopyran-2-carboxamide (Compound 138)

### Example 39A: Tert-Butyl (trans-1-(((6-chloro-1H-benzo[d]imidazol-2-yl)methyl)carbamoyl)cyclohexyl)carbamate

The reaction and purification conditions described in Example 2B substituting (6-chloro-1H-benzo[d]imidazol-2-yl)methanamine for the product of Example 2A, and trans-4-((tert-butoxycarbonyl)amino)cyclohexanecarboxylic acid for the product of Example 1B gave the title compound. MS (ESI⁺) m/z 407 (M+H)⁺.

### Example 39B: 6-chloro-N-[(1r,4r)-4-{[(6-chloro-1H-benzimidazol-2-yl)methyl]carbamoyl}cyclohexyl]-4-oxo-3,4-dihydro-2H-1-benzopyran-2-carboxamide

The reaction and purification conditions described in Example 1C substituting the product of Example 39A for the product of Example 1A, and 6-chloro-4-oxochroman-2-carboxylic acid for the product of Example 1B gave the title compound. ¹H NMR (500 MHz, DMSO-d₆) δ ppm 8.55 (t, J=5.5 Hz, 1H), 8.15 (d, J=8.0 Hz, 1H), 7.71 (d, J=2.0 Hz, 1H), 7.69-7.58 (m, 3H), 7.36 (dd, J=8.6, 2.0 Hz, 1H), 7.24-7.13 (m, 1H), 5.11 (dd, J=8.1, 5.5 Hz, 1H), 4.56 (d, J=5.5 Hz, 2H), 3.04-2.88 (m, 2H), 2.23-2.12 (m, 1H), 1.89-1.79 (m, 3H), 1.78-1.70 (m, 1H), 1.48-1.33 (m, 2H), 1.37-1.17 (m, 2H); MS (APCI⁺) m/z 515 (M+H)⁺.

### Example 40: 6-chloro-N-{(3R,6S)-6-[3-(4-chlorophenoxy)azetidine-1-carbonyl]oxan-3-yl}-4-oxo-3,4-dihydro-2H-1-benzopyran-2-carboxamide (Compound 139)

### Example 40A: Tert-Butyl ((3R,6S)-6-(3-(4-chlorophenoxy)azetidine-1-carbonyl)tetrahydro-2H-pyran-3-yl)carbamate

The methodologies described in Example 30D substituting (2S,5R)-5-((tert-butoxycarbonyl)amino)tetrahydro-2H-pyran-2-carboxylic acid (purchased from Astatech) for 3-(2-(4-chloro-3-fluorophenoxy)acetamido)bicyclo[1.1.1]pentane-1-carboxylic acid and substituting 3-(4-chlorophenoxy)azetidine (purchased from PharmaBlock) for Example 30C gave the title compound. MS (APCI⁺) m/z 411 (M+H)⁺.

### Example 40B: ((2S,5R)-5-aminotetrahydro-2H-pyran-2-yl)(3-(4-chlorophenoxy)azetidin-1-yl)methanone

The methodologies described in 21B substituting Example 40A for Example 21A gave the title compound. MS (APCI⁺) m/z 303 (M+H)⁺.

### Example 40C: 6-chlor-N-{(3R,6S)-6-[3-(4-chlorophenoxy)azetidine-1-carbonyl]oxan-3-yl}-4-oxo-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 40B for Example 30C gave the title compound. ¹H NMR (400 MHz, DMSO-d₆) δ ppm 8.20 (dt, J=7.8, 5.0 Hz, 1H), 7.68-7.59 (m, 2H), 7.39-7.30 (m, 2H), 7.16 (ddd, J=8.6, 1.4, 0.7 Hz, 1H), 6.92-6.83 (m, 2H), 5.14 (dd, J=7.6, 5.9 Hz, 1H), 5.02 (dp, J=7.6, 3.2, 2.5 Hz, 1H), 4.74-4.64 (m, 1H), 4.31 (dd, J=10.9, 6.5 Hz, 1H), 4.16 (dd, J=10.5, 3.3 Hz, 1H), 3.92-3.85 (m, 1H), 3.85-3.78 (m, 1H), 3.81-3.70 (m, 1H), 3.68 (s, 1H), 3.18-3.03 (m, 1H), 3.03-2.89 (m, 2H), 1.97-1.79 (m, 2H), 1.56 (s, 1H), 1.61-1.45 (m, 1H); MS (APCI⁺) m/z 520 (M+H)⁺.

### Example 41: 6-chloro-N-[(3R,6S)-6-{[(7-chloroimidazo[1,2-a]pyridin-2-yl)methyl]carbamoyl}oxan-3-yl]-4-oxo-3,4-dihydro-2H-1-benzopyran-2-carboxamide (Compound 140)

### Example 41A: Tert-Butyl ((3R,6S)-6-(((7-chloroimidazo[1,2-a]pyridin-2-yl)methyl)carbamoyl)tetrahydro-2H-pyran-3-yl)carbamate

The methodologies described in Example 30D substituting (2S,5R)-5-((tert-butoxycarbonyl)amino)tetrahydro-2H-pyran-2-carboxylic acid (purchased from Astatech) for 3-(2-(4-chloro-3-fluorophenoxy)acetamido)bicyclo[1.1.1]pentane-1-carboxylic acid, substituting (7-chloroimidazo[1,2-a]pyridin-2-yl)methanamine hydrochloride (purchased from Anichem) for Example 30C, and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. MS (APCI⁺) m/z 409 (M+H)⁺.

### Example 41B: (2S,5R)-5-amino-N-((7-chloroimidazo[1,2-a]pyridin-2-yl)methyl)tetrahydro-2H-pyran-2-carboxamide

The methodologies described in 21B substituting Example 41A for Example 21A gave the title compound. MS (APCI⁺) m/z 309 (M+H)⁺.

### Example 41C: 6-chloro-N-[(3R,6S)-6-{[(7-chloroimidazo[1,2-a]pyridin-2-yl)methyl]carbamoyl}oxan-3-yl]-4-oxo-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid, substituting Example 41B for Example 30C, and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (400 MHz, DMSO-d₆) δ ppm 8.54 (dd, J=7.2, 0.8 Hz, 1H), 8.23 (dd, J=7.9, 3.3 Hz, 1H), 8.11 (td, J=6.0, 2.3 Hz, 1H), 7.74 (s, 1H), 7.69-7.60 (m, 3H), 7.21-7.13 (m, 1H), 6.94 (dd, J=7.2, 2.1 Hz, 1H), 5.14 (td, J=6.7, 1.0 Hz, 1H), 4.38 (d, J=5.9 Hz, 2H), 3.87 (dddd, J=33.3, 10.6, 4.8, 1.9 Hz, 1H), 3.76 (ddd, J=19.9, 11.3, 3.3 Hz, 2H), 3.17 (dt, J=21.0, 10.5 Hz, 1H), 3.01-2.94 (m, 2H), 2.03 (ddt, J=13.5, 8.0, 2.6 Hz, 1H), 1.97-1.80 (m, 1H), 1.69-1.40 (m, 2H); MS (APCI⁺) ml 517 (M+H)⁺.

### Example 42: 6-chloro-N-{(1r,4r)-4-[2-(4-chloro-3-fluorophenoxy)acetamido]cyclohexyl}-4-hydroxy-3,4-dihydro-2H-1-benzopyran-2-carboxamide (Compound 141)

To a solution of Example 31 (0.012 g, 0.024 mmol) in acetonitrile (0.16 mL) was added zinc chloride (0.010 g, 0.071 mmol). After stirring at 50° C. for 5 minutes, sodium cyanoborohydride (0.005 g, 0.071 mmol) was added, and this mixture was allowed to stir at 50° C. for 3 days. Then the reaction mixture was cooled to ambient temperature, diluted with N,N-dimethylformamide/water (1.2 mL, 3:1) and purified by preparative HPLC (Phenomenex® Luna® C18(2) 10 μm 100 Å AXIA™ column (250 mm×50 mm) using a 30-100% gradient of acetonitrile (A) and 0.1% trifluoroacetic acid in water (B) over 25 minutes, at a flow rate of 50 mL/minute) to give the title compound (0.006 g, 0.012 mmol, 50% yield). ¹H NMR (400 MHz, DMSO-d₆, dr 2.5:1) δ ppm 8.17 (d, J=7.9 Hz, 1H), 7.96 (t, J=7.1 Hz, 2H), 7.89 (d, J=8.1 Hz, 0.2H), 7.67-7.59 (m, 2H), 7.49 (td, J=8.9, 2.2 Hz, 1H), 7.40-7.35 (m, 0.2H), 7.23-7.14 (m, 1H), 7.06 (dt, J=11.5, 2.9 Hz, 2H), 6.92-6.81 (m, 2H), 6.51 (s, 0.2), 5.69 (d, J=6.4 Hz, 0.2H), 5.11 (dd, J=8.1, 5.4 Hz, 1H), 4.81 (m, 0.4H), 4.66-4.57 (m, 0.4H), 4.49 (d, J=3.7 Hz, 3H), 3.59 (s, 5H), 2.96 (dd, J=6.7, 3.9 Hz, 2H), 1.78 (s, 7H), 1.70 (s, 2H), 1.37-1.28 (m, 8H); MS (ESI⁺) m/z 493 (M−H₂O+H)⁺.

### Example 43: (2R,4R)-6-chloro-N-(3-{5-[(3,5-dimethylphenoxy)methyl]-2-oxo-1,3-oxazolidin-3-yl}bicyclo[1.1.1]pentan-1-yl)-4-hydroxy-3,4-dihydro-2H-1-benzopyran-2-carboxamide (Compound 142)

The reaction and purification conditions described in Example 6C substituting the product of Example 1C for the product of Example 6B gave the title compound. ¹H NMR (400 MHz, DMSO-d₆) δ ppm 8.77 (s, 1H), 7.38 (dd, J=2.7, 1.0 Hz, 1H), 7.20 (ddd, J=8.7, 2.7, 0.7 Hz, 1H), 6.89 (d, J=8.7 Hz, 1H), 6.60 (br s, 1H), 6.57 (br s, J=1.5 Hz, 2H), 5.72 (d, J=6.3 Hz, 1H), 4.88-4.76 (m, 2H), 4.62 (dd, J=12.0, 2.2 Hz, 1H), 4.13 (dd, J=11.0, 3.3 Hz, 1H), 4.06 (dd, J=11.0, 5.5 Hz, 1H), 3.70 (t, 0.1=8.9 Hz, 1H), 3.44-3.37 (m, 1H), 2.36 (ddd, J=13.0, 6.0, 2.5 Hz, 1H), 2.32 (s, 6H), 2.23 (s, 6H), 1.77-1.63 (m, 1H); MS (APCI⁺) m/z 495 (M−H₂O+H)⁺.

### Example 44: (2R,4R)-6-chloro-N-{2-[(4-chloro-3-fluorophenoxy)acetyl]-2-azaspiro[3.3]heptan-6-yl}-4-hydroxy-3,4-dihydro-2H-1-benzopyran-2-carboxamide (Compound 143)

### Example 44A: Tert-Butyl (2-(2-(4-chloro-3-fluorophenoxy)acetyl)-2-azaspiro[3.3]heptan-6-yl)carbamate

The reaction and purification conditions described in Example 2B substituting 2-(4-chloro-3-fluorophenoxy)acetic acid (CombiBlocks) for the product of Example 1B, and tert-butyl 2-azaspiro[3.3]heptan-6-ylcarbamate (Enamine) for the product of Example 2A gave the title compound. MS (APCI⁺) m/z 399 (M+H)⁺.

### Example 44B: (2R,4R)-6-chloro-N-(2-[(4-chloro-3-fluorophenoxy)acetyl]-2-azaspiro[3.3]heptan-6-yl)-4-hydroxy-3,4-dihydro-2H-1-benzopyran-2-carboxamide

The reaction and purification conditions described in Example 3C substituting the product of Example 44A for the product of Example 1A gave the title compound. ¹H NMR (400 MHz, DMSO-d₆) δ ppm 8.26 (t, J=8.4 Hz, 1H), 7.47 (td, 0.1=8.9, 1.5 Hz, 1H), 7.38 (d, J=2.7 Hz, 1H), 7.24-7.17 (m, 1H), 7.06 (ddd, J=11.3, 5.3, 2.8 Hz, 1H), 6.88 (d, J=8.7 Hz, 1H), 6.86-6.77 (m, 1H), 5.69 (d, J=5.3 Hz, 1H), 4.81 (dt, J=10.9, 5.5 Hz, 1H), 4.66-4.56 (m, 3H), 4.31-4.10 (m, 3H), 3.97 (s, 1H), 3.86 (s, 1H), 2.50-2.43 (m, 2H), 2.34 (ddd, J=12.3, 6.0, 3.2 Hz, 1H), 2.29-2.21 (m, 2H), 1.76-1.62 (m, 1H); MS (APCI⁻) m/z 507 (M−H)⁻.

### Example 45: 2-(4-chloro-3-fluorophenoxy)-N-[2-(6-chloro-4-oxo-3,4-dihydro-2H-1-benzopyran-2-carbonyl)-2-azaspiro[3.3]heptan-6-yl]acetamide (Compound 144)

### Example 45A: Tert-Butyl 6-(2-(4-chloro-3-fluorophenoxy)acetamido)-2-azaspiro[3.3]heptane-2-carboxylate

The reaction and purification conditions described in Example 2B substituting 2-(4-chloro-3-fluorophenoxy)acetic acid (Pharmablock) for the product of Example 1B, and tert-butyl 6-amino-2-azaspiro[3.3]heptane-2-carboxylate (Synnovator) for the product of Example 2A gave the title compound. MS (ESI⁺) m/z 343 (M−C(CH₃)₃+H)⁺.

### Example 45B: 2-(4-chloro-3-fluorophenoxy)-N-(2-azaspiro[3.3]heptan-6-yl)acetamide, 3 Trifluoroacetic Acid

The product of Example 45A (1.55 g, 3.89 mmol) was dissolved in dichloromethane (20 mL) and stirred at 0° C. Trifluoroacetic acid (5 mL) was added in one portion. The reaction mixture was slowly warmed up to ambient temperature over 20 minutes and stirred for one hour. The mixture was then concentrated under reduced pressure to give the title compound (2.5 g, 3.90 mmol, 100% yield). MS (APCI⁺) ml 299 (M+H)⁺.

### Example 45C: 2-(4-chloro-3-fluorophenoxy)-N-[2-(6-chloro-4-oxo-3,4-dihydro-2H-1-benzopyran-2-carbonyl)-2-azaspiro[3.3]heptan-6-yl]acetamide

The reaction and purification conditions described in Example 2B substituting the product of Example 45B for the product of Example 2A, and 6-chloro-4-oxochroman-2-carboxylic acid for the product of Example 1B gave the title compound. ¹H NMR (400 MHz, Chloroform-d₆) δ ppm 7.86 (t, J=2.2 Hz, 1H), 7.45 (td, J=8.5, 2.6 Hz, 1H), 7.33 (t, J=8.6 Hz, 1H), 6.97 (dd, J=10.3, 8.8 Hz, 1H), 6.75 (dd, J=10.2, 2.9 Hz, 1H), 6.71-6.63 (m, 1H), 6.56 (dd, J=12.3, 7.7 Hz, 1H), 5.00 (td, J=10.7, 4.0 Hz, 1H), 4.43 (s, 2H), 4.52-4.29 (m, 3H), 4.17-4.13 (m, 1H), 4.06-3.98 (m, 1H), 3.08 (ddd, J=17.2, 10.8, 1.5 Hz, 1H), 2.93 (ddd, J=17.3, 6.7, 4.0 Hz, 1H), 2.75-2.65 (m, 2H), 2.30-2.17 (m, 2H); MS (APCI⁺) m/z 507 (M+H)⁺.

### Example 46: 2-(4-chloro-3-fluorophenoxy)-N-{2-[rac-(2R,4R)-6-chloro-4-hydroxy-3,4-dihydro-2H-1-benzopyran-2-carbonyl]-2-azaspiro[3.3]heptan-6-yl}acetamide (Compound 145)

The reaction and purification conditions described in Example 6C substituting the product of Example 45C for the product of Example 6B gave the title compound. ¹H NMR (400 MHz, Chloroform-d₆) δ ppm 7.43 (d, J=2.6 Hz, 1H), 7.33 (td, J=8.6, 1.9 Hz, 1H), 7.16 (td, J=8.4, 2.6 Hz, 1H), 6.83-6.71 (m, 2H), 6.70-6.63 (m, 1H), 6.53 (t, J=9.3 Hz, 1H), 4.91-4.81 (m, 1H), 4.74-4.65 (m, 1H), 4.42 (s, 2H), 4.46-4.30 (m, 3H), 4.09 (q, J=10.4 Hz, 1H), 4.03-3.90 (m, 2H), 2.74-2.60 (m, 2H), 2.45 (q, J=4.7 Hz, 2H), 2.28-2.12 (m, 2H); MS (APCI⁺) m/z 491 (M−H₂O+H)⁺.

### Example 47: 6-chloro-N-[(3S)-3-hydroxy-4-{[(1s,3R)-3-(trifluoromethoxy)cyclobutane-1-carbonyl]amino}bicyclo[2.2.2]octan-1-yl]-4-oxo-4H-1-benzopyran-2-carboxamide (Compound 146)

### Example 47A: (S)-tert-butyl (4-(6-chloro-4-oxo-4H-chromene-2-carboxamido)-2-hydroxybicyclo[2.2.2]octan-1-yl)carbamate

The methodologies described in Example 30D substituting 6-chloro-4-oxo-4H-chromene-2-carboxylic acid (purchased from Princeton Bio) for 3-(2-(4-chloro-3-fluorophenoxy)acetamido)bicyclo[1.1.1]pentane-1-carboxylic acid, substituting (S)-tert-butyl (4-amino-2-hydroxybicyclo[2.2.2]octan-1-yl)carbamate hydrochloric acid (CALICO Life Sciences; AbbVie Inc.; Sidrauski, Carmela; et al. WO2017/193030, 2017, A1) for Example 30C, and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. MS (APCI⁺) m/z 463 (M+H)⁺.

### Example 47B: (S)—N-(4-amino-3-hydroxybicyclo[2.2.2]octan-1-yl)-6-chloro-4-oxo-4H-chromene-2-carboxamide

The methodologies described in Example 21B substituting Example 47A for Example 21A gave the title compound. MS (APCI⁺) m/z 363 (M+H)⁺.

### Example 47C: 6-chloro-N-[(3S)-3-hydroxy-4-{[(1s,3R)-3-(trifluoromethoxy)cyclobutane-1-carbonyl]amino}bicyclo[2.2.2]octan-1-yl]-4-oxo-4H-1-benzopyran-2-carboxamide

To a solution of Example 25N (0.040 g, 0.15 mmol) in methanol (2.2 mL) was added sodium hydroxide (0.23 mL, 0.58 mmol, 2.5M aqueous). After stirring at 50° C. for 10 minutes, the reaction mixture was concentrated in vacuo, diluted with a drop of acetonitrile and concentrated HCl, and concentrated again. The residue was taken up in N,N-dimethylformamide (2.2 mL) and triethylamine (0.16 mL, 1.2 mmol). This suspension was then added to Example 47B (0.053 g, 0.15 mmol), followed by the addition of 1-[bis(dimethylamino)methylene]-1H-1,2,3-triazolo[4,5-b]pyridinium 3-oxid hexafluorophosphate (HATU, 0.072 g, 0.19 mmol). The reaction mixture was stirred for 24 hours, was diluted with water (0.3 mL), and then was concentrated in vacuo. The residue was taken up in N,N-dimethylformamide (3 mL), and purified by preparative HPLC [YMC TriArt™ C18 Hybrid 5 μm column, 50×100 mm, flow rate 140 mL/minute, 5-100% gradient of acetonitrile in buffer (1% TFA)] to give the title compound (0.093 g, 0.176 mmol, 121% yield). ¹H NMR (500 MHz, DMSO-d₆) δ ppm 8.24 (s, 1H), 7.99-7.91 (m, 2H), 7.86 (d, J=9.0 Hz, 1H), 7.33 (s, 1H), 6.81 (s, 1H), 6.54 (s, 1H), 5.19 (d, J=3.8 Hz, 1H), 4.73 (p, J=7.6 Hz, 1H), 4.12 (d, J=9.3 Hz, 1H), 3.33 (s, 4H), 2.70-2.59 (m, 1H), 2.42 (q, J=6.4 Hz, 2H), 2.38-2.17 (m, 3H), 2.09-1.92 (m, 2H), 1.90-1.84 (m, 3H), 1.73 (dt, J=13.0, 6.6 Hz, 1H), MS (APCI⁺) m/z 529 (M+H)⁺.

### Example 48: (2S,4S)-6-chloro-4-hydroxy-N-[(3S)-3-hydroxy-4-{[(1s,3R)-3-(trifluoromethoxy)cyclobutane-1-carbonyl]amino}bicyclo[2.2.2]octan-1-yl]-3,4-dihydro-2H-1-benzopyran-2-carboxamide and (2R,4R)-6-chloro-4-hydroxy-N-[(3S)-3-hydroxy-4-{[(1s,3R)-3-(trifluoromethoxy)cyclobutane-1-carbonyl]amino}bicyclo[2.2.2]octan-1-yl]-3,4-dihydro-2H-1-benzopyran-2-carboxamide (Compound 147)

The methodologies described in Example 5 substituting Example 47 for Example 4 and purifying by preparative HPLC (Phenomenex® Luna® C18(2) 10 μm 100 Å AXIA™ column (250 mm×50 mm) using a 30-100% gradient of acetonitrile (A) and 0.1% trifluoroacetic acid in water (B) over 25 minutes, at a flow rate of 50 mL/minute) gave the title compounds. ¹H NMR (400 MHz, Chloroform-d, dr 10:1) δ ppm 7.42 (d, J=2.6 Hz, 1H), 7.32 (d, J=2.6 Hz, 0H), 7.21 (dd, J=8.8, 2.6 Hz, 0H), 7.17 (dd, J=8.7, 2.6 Hz, 1H), 6.86 (dd, J=8.7, 1.8 Hz, 0H), 6.81 (dd, J=8.7, 2.4 Hz, 1H), 6.35 (s, OH), 6.27 (s, 1H), 5.27 (s, 1H), 4.87 (t, J=6.2 Hz, 1H), 4.77 (s, OH), 4.61 (s, OH), 4.54 (tt, J=8.1, 4.7 Hz, 2H), 4.08 (d, J=8.6 Hz, 1H), 2.66-2.50 (m, 2H), 2.54-2.39 (m, 3H), 2.23-2.00 (m, 1H), 1.99-1.79 (m, 2H), 1.79-1.68 (m, 2H), 1.56 (dq, J=11.8, 6.0 Hz, 1H); MS (APCI⁺) m/z 515 (M−H₂O+H)⁺.

### Example 49: 6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide (Compound 148)

### Example 49A: Tert-Butyl (3-(4-(3,4-difluorophenyl)-1H-imidazol-1-yl)bicyclo[1.1.1]pentan-1-yl)carbamate

Modifying a reported preparation of imidazoles (Sumitomo Dainippon Pharma Co, Ltd, etc. EP2905279, 2015, A1), to a solution of 3,4-difluorobenzaldehyde (0.78 mL, 7.0 mmol) in ethanol (30 mL) and tetrahydrofuran (9 mL) was added toluene-4-sulfonylmethyl isocyanide (TOSMIC, 1.51 g, 7.74 mmol), followed by a solution of sodium cyanide (0.038 g, 0.77 mmol) in a few drops of water. This reaction mixture was allowed to stir at ambient temperature for 4 hours, was concentrated, diluted with ethyl acetate, and concentrated again to provide an impure residue containing 5-(3,4-difluorophenyl)-4-tosyl-4,5-dihydrooxazole.

To a solution of 5-(3,4-difluorophenyl)-4-tosyl-4,5-dihydrooxazole (2.00 g, 5.93 mmol) in xylene (12 mL) was added tert-butyl (3-aminobicyclo[1.1.1]pentan-1-yl)carbamate (1.93 g, 9.72 mmol). This reaction mixture was allowed to stir at 135° C. for 4.5 hours, was cooled to ambient temperature, and concentrated in vacuo. The residue was diluted with N,N-dimethylformamide (6 mL), and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound. ¹H NMR (500 MHz, DMSO-d₆) δ ppm 8.72 (s, 1H), 8.16 (d, J=1.5 Hz, 1H), 7.90-7.78 (m, 2H), 7.75-7.51 (m, 2H), 2.47 (s, 6H), 1.41 (s, 9H); MS (APCI⁺) ml 362 (M+H)⁺.

### Example 49B: 3-(4-(3,4-difluorophenyl)-1H-imidazol-1-yl)bicyclo[1.1.1]pentan-1-amine

The methodologies described in Example 21B substituting Example 49A for Example 21A gave the title compound as a trifluoroacetic acid salt. MS (APCI⁺) m/z 262 (M+H)⁺.

### Example 49C: 6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 49B for Example 30C gave the title compound. ¹H NMR (500 MHz, DMSO-d₆) δ ppm 9.22 (s, 1H), 8.50-8.46 (m, 1H), 8.09 (d, J=1.5 Hz, 1H), 7.85-7.75 (m, 1H), 7.69-7.67 (m, 1H), 7.66-7.60 (m, 2H), 7.52 (dt, J=10.7, 8.5 Hz, 1H), 7.19 (dd, J=8.4, 0.9 Hz, 1H), 5.17 (dd, J=8.7, 5.7 Hz, 1H), 3.03-2.97 (m, 2H), 2.58 (s, 6H); MS (APCI⁺) m/z 470 (M+H)⁺.

### Example 50: rac-(2R,4R)-6-chloro-N-{3-[4-(3,4-difluorophenyl)-1H-imidazo-1-yl]bicyclo[1.1.1]pentan-1-yl}-4-hydroxy-3,4-dihydro-2H-1-benzopyran-2-carboxamide (Compound 149)

The methodologies described in Example 5 substituting Example 49 for Example 4 and purifying by preparative HPLC (Phenomenex® Luna® C18(2) 10 μm 100 Å AXIA™ column (250 mm×50 mm) using a 30-100% gradient of acetonitrile (A) and 0.1% trifluoroacetic acid in water (B) over 25 minutes, at a flow rate of 50 mL/minute) gave the title compound. ¹H NMR (400 MHz, DMSO-d₆, dr 25:1) δ ppm 8.99 (s, 1H), 8.71 (d, J=1.4 Hz, 1H), 8.18 (d, J=1.5 Hz, 1H), 7.85 (ddd, J=11.9, 7.7, 2.2 Hz, 1H), 7.68-7.61 (m, 1H), 7.55 (dt, J=10.6, 8.5 Hz, 1H), 7.40 (dd, J=2.7, 1.0 Hz, 1H), 7.34 (d, J=2.6 Hz, 0.04H), 7.27 (dd, J=8.7, 2.7 Hz, 0.05H), 7.22 (ddd, J=8.8, 2.7, 0.7 Hz, 1H), 6.91 (d, J=8.7 Hz, 1H), 4.88-4.81 (m, 1H), 4.68 (dd, J=11.9, 2.4 Hz, 1H), 4.64-4.59 (m, 0.12H), 2.63 (s, 6H), 2.40 (ddd, J=12.9, 6.0, 2.4 Hz, 1H), 1.75 (ddd, J=12.9, 12.0, 10.7 Hz, 1H); MS (APCI⁺) m/z 472 (M+H)⁺.

### Example 51: 6-chloro-4-oxo-N-(4-{5-[(1s,3s)-3-(trifluoromethoxy)cyclobutyl]-1,3,4-oxadiazol-2-yl}bicyclo[2.1.1]hexan-1-yl)-3,4-dihydro-2H-1-benzopyran-2-carboxamide (Compound 150)

### Example 51A: (1s,3s)-3-(trifluoromethoxy)cyclobutanecarbohydrazide

To a suspension of Example 25N (0.10 g, 0.37 mmol) in ethanol (1.5 mL) was added hydrazine hydrate (0.18 mL, 1.8 mmol, 50 weight %), and the reaction mixture was heated at 90° C. overnight. Then the reaction mixture was cooled to ambient temperature and concentrated. The residue was purified by silica gel column chromatography (0-100% ethyl acetate/heptanes) and visualized by KMnO₄ thin-layer chromatography stain to give the title compound (0.067 g, 0.34 mmol, 93% yield). ¹H NMR (400 MHz, Chloroform-d) δ ppm 6.67 (s, 1H), 4.56 (p, J=7.6 Hz, 1H), 3.92-3.89 (m, 2H), 2.60-2.54 (m, 4H), 2.54-2.42 (m, 1H); MS (APCI⁺) m/z 199 (M+H)⁺.

### Example 51B: Tert-Butyl (4-(2-((1s,3s)-3-(trifluoromethoxy)cyclobutanecarbonyl)hydrazinecarbonyl)bicyclo[2.1.1]hexan-1-yl)carbamate

The methodologies described in Example 25P substituting Example 51A for Example 25L and substituting 4-((tert-butoxycarbonyl)amino)bicyclo[2.1.1]hexane-1-carboxylic acid for Example 250 gave the title compound. ¹H NMR (400 MHz, Chloroform-d) δ ppm 8.72 (s, 1H), 8.36 (d, J=6.2 Hz, 1H), 4.99 (s, 1H), 4.59 (p, J=7.5 Hz, 1H), 2.71-2.62 (m, 1H), 2.58 (t, J=7.9 Hz, 4H), 1.96-1.87 (m, 3H), 1.73 (dd, J=3.9, 1.9 Hz, 2H), 1.58 (s, 3H), 1.45 (s, 9H); MS (APCI⁺) m/z 422 (M+H)⁺.

### Example 51C: Tert-Butyl (4-(5-((1s,3s)-3-(trifluoromethoxy)cyclobutyl)-1,3,4-oxadiazol-2-yl)bicyclo[2.1.1]hexan-1-yl)carbamate

The methodologies described in Example 25Q substituting Example 51B for Example 25P gave the title compound. MS (APCI⁺) m/z 404 (M+H)⁺.

### Example 51D: 4-(5-((1s,3s)-3-(trifluoromethoxy)cyclobutyl)-1,3,4-oxadiazol-2-yl)bicyclo[2.1.1]hexan-1-amine

The methodologies described in Example 21B substituting Example 51C for Example 21A gave the title compound. MS (APCI⁺) m/z 304 (M+H)⁺.

### Example 51E: 6-chloro-4-oxo-N-(4-{5-[(1s,3s)-3-(trifluoromethoxy)cyclobutyl]-1,3,4-oxadiazol-2-yl}bicyclo[2.1.1]hexan-1-yl)-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 51D for Example 30C gave the title compound. ¹H NMR (400 MHz, Chloroform-d) δ ppm 7.90 (d, J=2.7 Hz, 1H), 7.50 (dd, J=8.8, 2.7 Hz, 1H), 7.07 (d, J=8.8 Hz, 1H), 7.01 (s, 1H), 4.88 (dd, 0.1=13.4, 3.3 Hz, 1H), 4.77-4.65 (m, 1H), 3.33 (tt, J=10.1, 7.7 Hz, 1H), 3.20 (dd, J=17.3, 3.3 Hz, 1H), 2.95-2.79 (m, 3H), 2.75-2.63 (m, 2H), 2.63-2.55 (m, 2H), 2.30-2.17 (m, 2H), 2.20-2.06 (m, 2H), 2.10-1.90 (m, 2H); MS (APCI⁺) m/z 512 (M+H)⁺.

### Example 52: 6-chloro-4-oxo-N-(3-{5-[(1s,3s)-3-(trifluoromethoxy)cyclobutyl]-1,3,4-oxadiazol-2-yl}bicyclo[1.1.1]pentan-1-yl)-3,4-dihydro-2H-1-benzopyran-2-carboxamide (Compound 151)

### Example 52A: Methyl 3-(6-chlorochroman-2-carboxamido)bicyclo[1.1.1]pentane-1-carboxylate

The methodologies described in Example 30D substituting 6-chlorochroman-2-carboxylic acid (purchased from Anichem) for 3-(2-(4-chloro-3-fluorophenoxy)acetamido)bicyclo[1.1.1]pentane-1-carboxylic acid and substituting methyl 3-aminobicyclo[1.1.1]pentane-1-carboxylate hydrochloride for Example 30C gave the title compound. MS (APCI⁺) m/z 336 (M+H)⁺.

### Example 52B: 6-chloro-N-(3-(hydrazinecarbonyl)bicyclo[1.1.1]pentan-1-yl)chroman-2-carboxamide

The methodologies described in Example 51A substituting 52A for Example 25N gave the title compound. ¹H NMR (400 MHz, DMSO-d₆) δ ppm 9.04 (s, 1H), 8.62 (s, 1H), 7.17-7.10 (m, 2H), 6.91-6.82 (m, 1H), 4.45 (dd, J=9.2, 3.1 Hz, 1H), 4.19 (s, 2H), 2.79 (ddd, J=15.9, 9.8, 5.6 Hz, 1H), 2.66 (dt, J=16.7, 5.1 Hz, 1H), 2.16 (s, 6H), 2.11 (dt, J=8.5, 5.3 Hz, 1H), 1.89-1.75 (m, 1H); MS (APCI⁺) m/z 336 (M+H)⁺.

### Example 52C: 6-chloro-N-(3-(2-((1s,3)-3-(trifluoromethoxy)cyclobutanecarbonyl)hydrazinecarbonyl)bicyclo[1.1.1]pentan-1-yl)chroman-2-carboxamide

The methodologies described in Example 47C substituting Example 52B for Example 47B gave the title compound. ¹H NMR (600 MHz, Chloroform-d) δ ppm 8.13 (d, J=6.1 Hz, 1H), 8.07 (s, 1H), 7.11-7.05 (m, 2H), 6.97 (s, 1H), 6.82 (d, J=8.6 Hz, 1H), 4.61-4.55 (m, 1H), 4.42 (dd, J=10.1, 2.8 Hz, 1H), 2.86 (ddd, J=16.6, 10.7, 5.8 Hz, 1H), 2.75 (dt, J=16.5, 4.6 Hz, 1H), 2.60 (d, J=6.3 Hz, 5H), 2.46 (s, 6H), 2.45-2.37 (m, 1H), 2.00-1.90 (m, 1H); MS (APCI⁺) m/z 502 (M+H)⁺.

### Example 52D: 6-chloro-N-(3-(5-((1s,3s)-3-(trifluoromethoxy)cyclobutyl)-1,3,4-oxadiazol-2-yl)bicyclo[1.1.1]pentan-1-yl)chroman-2-carboxamide

The methodologies described in Example 25Q substituting Example 52C for Example 25P gave the title compound. ¹H NMR (400 MHz, Chloroform-d) δ ppm 7.14-7.04 (m, 3H), 6.83 (d, J=8.5 Hz, 1H), 4.71 (p, J=7.6 Hz, 1H), 4.45 (dd, J=10.1, 2.8 Hz, 1H), 3.34 (tt, J=10.1, 7.7 Hz, 1H), 2.95-2.76 (m, 4H), 2.76-2.66 (m, 3H), 2.65 (s, 5H), 2.49-2.37 (m, 1H), 2.04-1.90 (m, 1H); MS (APCI⁺) m/z 484 (M+H)⁺.

### Example 52E: 6-chloro-4-oxo-N-(3-{5-[(1s,3s)-3-(trifluoromethoxy)cyclobutyl]-1,3,4-oxadiazol-2-yl}bicyclo[1.1.1]pentan-1-yl)-3,4-dihydro-2H-1-benzopyran-2-carboxamide

The methodologies described in Example 4 substituting Example 52D for Example 30 gave the title compound and Example 60. ¹H NMR (500 MHz, Chloroform-d) δ ppm 7.90 (d, J=2.6 Hz, 1H), 7.50 (dd, J=8.8, 2.7 Hz, 1H), 7.10-7.03 (m, 2H), 4.87 (dd, J=13.6, 3.3 Hz, 1H), 4.71 (p, J=7.6 Hz, 1H), 3.33 (tt, J=10.2, 7.7 Hz, 1H), 3.20 (dd, J=17.3, 3.3 Hz, 1H), 2.93-2.82 (m, 3H), 2.76-2.64 (m, 2H), 2.67 (s, 6H); MS (APCI⁺) m/z 498 (M+H)⁺.

### Example 53: 6-chloro-4-oxo-N-[(3R,6)-6-{5-[(1s,3R)-3-(trifluoromethoxy)cyclobutyl]-1,3,4-oxadiazol-2-yl}oxan-3-yl]-3,4-dihydro-2H-1-benzopyran-2-carboxamide (Compound 152)

### Example 3A: Tert-Butyl ((3R,6S)-6-(2-((1s,3R)-3-(trifluoromethoxy)cyclobutanecarbonyl)hydrazinecarbonyl)tetrahydro-2H-pyran-3-yl)carbamate

The methodologies described in Example 25P substituting Example 51A for Example 25L and substituting (2S,5R)-5-((tert-butoxycarbonyl)amino)tetrahydro-2H-pyran-2-carboxylic acid (purchased from Astatech) for Example 250 gave the title compound. MS (APCI⁺) m/z 425 (M+H)⁺.

### Example 53B: Tert-Butyl ((3R,6S)-6-(5-((1s,3R)-3-(trifluoromethoxy)cyclobutyl)-1,3,4-oxadiazol-2-yl)tetrahydro-2H-pyran-3-yl)carbamate

The methodologies described in Example 25Q substituting Example 53A for Example 25P gave the title compound. ¹H NMR (400 MHz, Chloroform-d) δ ppm 4.70 (p, J=7.6 Hz, 1H), 4.62 (dd, J=9.9, 3.2 Hz, 1H), 4.43 (s, 1H), 4.16 (dd, J=11.0, 4.2 Hz, 1H), 3.75 (s, 1H), 3.41-3.21 (m, 2H), 2.87 (dtd, J=10.1, 7.4, 2.9 Hz, 1H), 2.72 (t, J=9.5 Hz, 2H), 2.29-2.01 (m, 3H), 1.45 (s, 9H); MS (APCI⁺) m/z 408 (M+H)⁺.

### Example 53C: (3R,6S)-6-(5-((s,3R)-3-(trifluoromethoxy)cyclobutyl)-1,3,4-oxadiazol-2-yl)tetrahydro-2H-pyran-3-amine

The methodologies described in Example 21B substituting Example 53B for Example 21A gave the title intermediate. MS (APCI⁺) m/z 308 (M+H)⁺.

### Example 3D: 6-chloro-4-oxo-N-[(3R,6S)-6-{5-[(1s,3R)-3-(trifluoromethoxy)cyclobutyl]-1,3,4-oxadiazol-2-yl}oxan-3-yl]-3,4-dihydro-2H-1-benzopyran-2-carboxamide

The methodologies described in Example 30D substituting 6-chloro-4-oxochroman-2-carboxylic acid (purchased from Princeton Bio) for 3-(2-(4-chloro-3-fluorophenoxy)acetamido)bicyclo[1.1.1]pentane-1-carboxylic acid and substituting Example 53C for Example 30C gave the title compound. ¹H NMR (400 MHz, Chloroform-d) δ ppm 7.89 (dd, J=2.7, 1.1 Hz, 1H), 7.50 (ddd, J=8.9, 2.7, 0.9 Hz, 1H), 7.06 (dd, J=8.8, 1.7 Hz, 1H), 6.58 (dd, J=7.9, 2.9 Hz, 1H), 4.92 (dd, J=13.0, 3.4 Hz, 1H), 4.78-4.66 (m, 2H), 4.24-4.14 (m, 1H), 4.18-4.10 (m, 1H), 3.50-3.30 (m, 2H), 3.20 (dd, J=17.3, 3.4 Hz, 1H), 2.89 (tdd, J=13.0, 9.4, 6.1 Hz, 3H), 2.78-2.64 (m, 2H), 2.38-2.07 (m, 2H), 1.73 (dddd, J=20.6, 18.0, 10.1, 4.8 Hz, 1H), 1.41 (s, 1H); MS (APCI⁺) m/z 516 (M+H)⁺.

### Example 54: 2-(4-chloro-3-fluorophenoxy)-N-[(3R,6S)-6-{5-[(1s,3R)-3-(trifluoromethoxy)cyclobutyl]-1,3,4-oxadiazol-2-yl}oxan-3-yl]acetamide (Compound 153)

The methodologies described in Example 30 substituting 2-(4-chloro-3-fluorophenoxy)acetic acid for 3-(2-(4-chloro-3-fluorophenoxy)acetamido)bicyclo[1.1.1]pentane-1-carboxylic acid and substituting Example 53C for Example 30C gave the title compound. ¹H NMR (400 MHz, Chloroform-d) δ ppm 7.34 (t, J=8.6 Hz, 1H), 6.77 (dd, J=10.2, 2.8 Hz, 1H), 6.69 (ddd, J=8.9, 2.9, 1.2 Hz, 1H), 6.38 (d, J=7.9 Hz, 1H), 4.77-4.65 (m, 2H), 4.47 (s, 2H), 4.22-4.12 (m, 2H), 3.43-3.28 (m, 2H), 2.93-2.82 (m, 2H), 2.78-2.64 (m, 1H), 2.27 (dd, J=13.5, 4.5 Hz, 1H), 2.22-2.06 (m, 2H), 1.77-1.58 (m, 1H); MS (APCI⁺) m/z 494 (M+H)⁺.

### Example 55: (2R,4R)-6-chloro-N-(3-{3-[(4-chloro-3-fluorophenoxy)methyl]-4,5-dihydro-1,2,4-oxadiazol-5-yl}bicyclo[1.1.1]pentan-1-yl)-4-hydroxy-3,4-dihydro-2H-1-benzopyran-2-carboxamide (Compound 154)

The purification conditions of Example 57 also afforded this title compound (as an earlier eluting fraction). ¹H NMR (400 MHz, DMSO-d₆) δ ppm 8.65 (s, 1H), 7.51 (t, J=8.9 Hz, 1H), 7.45 (d, J=1.7 Hz, 1H), 7.37 (dd, J=2.8, 1.0 Hz, 1H), 7.19 (dd, J=8.7, 2.7 Hz, 1H), 7.14 (dd, J=11.3, 2.9 Hz, 1H), 6.93-6.89 (m, 1H), 6.87 (d, J=8.7 Hz, 1H), 5.74 (s, 1H), 5.75-5.70 (m, 1H), 5.53 (d, J=1.3 Hz, 1H), 4.82-4.71 (m, 2H), 4.57 (dd, J=12.0, 2.2 Hz, 1H), 2.34 (ddd, J=12.9, 5.9, 2.3 Hz, 1H), 1.90 (s, 6H), 1.67 (td, J=12.5, 10.8 Hz, 1H); MS (APCI⁺) m/z 504 (M−H₂O+H)⁺.

### Example 56: (2R)-6-chloro-N-(3-{3-[(4-chloro-3-fluorophenoxy)methyl]-1,2,4-oxadiazol-5-yl}bicyclo[1.1.1]pentan-1-yl)-4-oxo-3,4-dihydro-2H-1-benzopyran-2-carboxamide (Compound 155)

The reaction and purification conditions described in Example 2B substituting 3-(3-((4-chloro-3-fluorophenoxy)methyl)-1,2,4-oxadiazol-5-yl)bicyclo[1.1.1]pentan-1-amine (prepared as described in International Patent Publication WO2017/193030 A1) for the product of Example 2A gave the title compound. ¹H NMR (400 MHz, DMSO-d₆) δ ppm 9.18 (s, 1H), 7.69-7.61 (m, 2H), 7.52 (t, J=8.9 Hz, 1H), 7.22 (dd, J=11.4, 2.9 Hz, 1H), 7.20-7.16 (m, 1H), 6.94 (ddd, J=9.0, 2.9, 1.2 Hz, 1H), 5.31 (s, 2H), 5.13 (dd, J=7.7, 6.6 Hz, 1H), 2.98 (d, J=1.3 Hz, 1H), 2.97 (s, 1H), 2.53 (s, 6H); MS (APCI⁺) m/z 518 (M+H)⁺.

### Example 57: (2R,4R)-6-chloro-N-(3-{3-[(4-chloro-3-fluorophenoxy)methyl]-1,2,4-oxadiazol-5-yl}bicyclo[1.1.1]pentan-1-yl)-4-hydroxy-3,4-dihydro-2H-1-benzopyran-2-carboxamide (Compound 156)

The product of Example 56 (24 mg, 0.046 mmol) was combined with methanol (1 mL) and stirred at ambient temperature. Sodium borohydride (10.5 mg, 0.28 mmol) was added. After stirring at ambient temperature for 20 minutes, saturated ammonium chloride solution (0.1 mL) was added. After stirring for another 10 minutes, the resulting mixture was combined with diatomaceous earth (5 g) and concentrated under reduced pressure to a free flowing powder and the powder was directly purified by reversed-phase flash chromatography [Interchim PuriFlash C18XS 15 μm 120 g column, flow rate 40 mL/minute, 5-100% gradient of acetonitrile in buffer (0.025 M aqueous ammonium bicarbonate, adjusted to pH 10 with ammonium hydroxide)] to give the title compound (12 mg, 0.023 mmol, 50% yield) as a later eluting fraction relative to Example 55. ¹H NMR (400 MHz, DMSO-d₆) δ ppm 8.93 (s, 1H), 7.51 (t, J=8.9 Hz, 1H), 7.38 (dd, J=2.7, 1.0 Hz, 1H), 7.22 (dd, J=4.5, 2.8 Hz, 1H), 7.21-7.18 (m, 1H), 6.94 (ddd, J=9.0, 2.9, 1.2 Hz, 1H), 6.89 (d, J=8.8 Hz, 1H), 5.75 (br s, 1H), 5.30 (s, 2H), 4.81 (dd, J=10.6, 5.9 Hz, 1H), 4.63 (dd, J=12.0, 2.3 Hz, 1H), 2.55 (s, 6H), 2.37 (ddd, J=12.9, 5.9, 2.4 Hz, 1H), 1.77-1.64 (m, 1H); MS (APCI⁺) m/z 502 (M−H₂O+H)⁺.

### Example 58: 4-{2-[(1s,3s)-3-(trifluoromethoxy)cyclobutyl]oxy}acetamido)-N-[5-(trifluoromethyl)pyridin-2-yl]methyl)bicyclo[2.2.2]octane-1-carboxamide (Compound 157)

### Example 58A: Tert-Butyl (4-(((5-(trifluoromethyl)pyridin-2-yl)methyl)carbamoyl)bicyclo[2.2.2]octan-1-yl)carbamate

The reaction and purification conditions described in Example 2B substituting (5-(trifluoromethyl)pyridin-2-yl)methanamine hydrochloride (Chem-Impex) for the product of Example 2A, and 4-((tert-butoxycarbonyl)amino)bicyclo[2.2.2]octane-1-carboxylic acid (Combi-Blocks) for the product of Example 1B gave the title compound. MS (APCI⁺) m/z 428 (M+H)⁺.

### Example 58B: 4-(2-((1s,3s)-3-(trifluoromethoxy)cyclobutoxy)acetamido)-N-((5-(trifluoromethyl)pyridin-2-yl)methyl)bicyclo[2.2.2]octane-1-carboxamide

The reaction and purification conditions described in Example 1C substituting the product of Example 58A for the product of Example 1A, and the product of Example 13P for the product of Example 1B gave the title compound. ¹H NMR (400 MHz, DMSO-d₆) δ ppm 8.89-8.84 (m, 1H), 8.22-8.12 (m, 2H), 7.37 (d, J=8.3 Hz, 1H), 7.02 (s, 1H), 4.48 (p, J=7.1 Hz, 1H), 4.40 (d, J=5.8 Hz, 2H), 3.70 (p, J=6.9 Hz, 1H), 3.69 (s, 2H), 2.79-2.68 (m, 2H), 2.18-2.07 (m, 2H), 1.89-1.75 (m, 12H); MS (APCI⁺) m/z 524 (M+H)⁺.

### Example 59: (1r,4r)-4-(2-{[(1s,3s)-3-(trifluoromethoxy)cyclobutyl]oxy}acetamido)-N-([5-(trifluoromethyl)pyridin-2-yl]methyl)cyclohexane-1-carboxamide (Compound 158)

### Example 59A: Tert-Butyl ((1r,4r)-4-(((5-(trifluoromethyl)pyridin-2-yl)methyl)carbamoyl)cyclohexyl)carbamate

The reaction and purification conditions described in Example 2B substituting (5-(trifluoromethyl)pyridin-2-yl)methanamine hydrochloride (Chem-Impex) for the product of Example 2A, and (1r,4r)-4-((tert-butoxycarbonyl)amino)cyclohexane-1-carboxylic acid (Enamine) for the product of Example 1B gave the title compound. MS (APCI⁺) m/z 402 (M+H)⁺.

### Example 59B: (1r,4r)-4-(2-{[(1s,3s)-3-(trifluoromethoxy)cyclobutyl]oxy}acetamido)-N-{[5-(trifluoromethyl)pyridin-2-yl]methyl}cyclohexane-1-carboxamide

The reaction and purification conditions described in Example 1C substituting the product of Example 59A for the product of Example 1A, and the product of Example 13P for the product of Example 1B gave the title compound. ¹H NMR (500 MHz, DMSO-d₆) δ ppm 8.55 (t, J=5.5 Hz, 1H), 8.15 (d, J=8.0 Hz, 1H), 7.71 (d, J=2.0 Hz, 1H), 7.69-7.58 (m, 3H), 7.36 (dd, J=8.6, 2.0 Hz, 1H), 7.24-7.13 (m, 1H), 5.11 (dd, J=8.1, 5.5 Hz, 1H), 4.56 (d, J=5.5 Hz, 2H), 3.04-2.88 (m, 2H), 2.23-2.12 (m, 1H), 1.89-1.79 (m, 3H), 1.78-1.70 (m, 1H), 1.48-1.33 (m, 2H), 1.37-1.17 (m, 2H); MS (APCI⁺) m/z 515 (M+H)⁺.

### Example 60: rac-(2R,4R)-6-chloro-4-hydroxy-N-(3-{5-[(1s,3S)-3-(trifluoromethoxy)cyclobutyl]-1,3,4-oxadiazol-2-yl}bicyclo[1.1.1]pentan-1-yl)-3,4-dihydro-2H-1-benzopyran-2-carboxamide (Compound 159)

The methodologies described in Example 4 substituting Example 52D for Example 30 gave the title compound and Example 52. ¹H NMR (500 MHz, DMSO-d₆) δ ppm 8.99 (s, 1H), 7.32 (d, J=2.7 Hz, 1H), 7.26 (dd, J=8.8, 2.7 Hz, 1H), 6.96 (d, J=8.7 Hz, 1H), 4.90 (p, J=7.5 Hz, 1H), 4.61 (t, J=3.7 Hz, 1H), 4.57 (dd, J=11.1, 2.7 Hz, 1H), 3.44-3.33 (m, 2H), 2.92-2.78 (m, 3H), 2.49 (s, 6H), 2.17-2.08 (m, 1H), 1.91 (ddd, J=14.2, 10.8, 3.7 Hz, 1H); MS (APCI⁺) m/z 500 (M+H)⁺.

### Example 61: rac-(2R,4R)-6-chloro-4-hydroxy-N-(4-{5-[(1s,3S)-3-(trifluoromethoxy)cyclobutyl]-1,3,4-oxadiazol-2-yl}bicyclo[2.1.1]hexan-1-yl)-3,4-dihydro-2H-1-benzopyran-2-carboxamide (Compound 160)

The methodologies described in Example 5 substituting Example 51 for Example 4 and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (400 MHz, Chloroform-d, dr 33:1) δ ppm 7.44 (d, J=2.6 Hz, 1H), 7.40 (t, J=3.1 Hz, 0.03H), 7.21 (dd, J=8.8, 2.6 Hz, 0.03H), 7.16 (dd, J=8.8, 2.6 Hz, 1H), 7.08 (s, 1H), 7.04 (d, J=5.4 Hz, 0.03H), 6.90 (d, J=8.8 Hz, 0.03H), 6.83 (d, J=8.7 Hz, 1H), 4.94 (dd, J=8.6, 5.4 Hz, 1H), 4.81 (d, J=3.2 Hz, 0.03H), 4.70 (p, J=7.5 Hz, 1H), 4.64 (dd, J=9.6, 3.2 Hz, 1H), 3.34 (tt, J=10.1, 7.7 Hz, 1H), 2.86 (dtt, J=9.9, 7.5, 2.5 Hz, 2H), 2.66 (dddq, J=13.8, 8.8, 5.6, 3.0 Hz, 3H), 2.58-2.48 (m, 2H), 2.22-1.94 (m, 8H); MS (APCI⁺) m/z 514 (M+H)⁺.

### Example 62: (2RS,4RS)-6-chloro-4-hydroxy-N-[(3R,6)-6-{5-[cis-3-(trifluoromethoxy)cyclobutyl]-1,3,4-oxadiazol-2-yl}oxan-3-yl]-3,4-dihydro-2H-1-benzopyran-2-carboxamide (Compound 161)

To a suspension of Example 53 (0.070 g, 0.14 mmol) in methanol (2.4 mL) was added sodium borohydride (0.026 g, 0.68 mmol). This mixture was allowed to stir at ambient temperature for 1 hour and then was quenched with a drop of water and concentrated under heated N₂. The residue was diluted with N,N-dimethylformamide (2 mL) and water (0.5 mL), filtered, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.070 g, 0.14 mmol, quantitative yield). ¹H NMR (500 MHz, CDCl₃) δ ppm 7.45 (td, J=2.4, 0.9 Hz, 1H), 7.19 (dddd, J=8.7, 2.6, 1.3, 0.6 Hz, 1H), 6.86 (dd, J=8.7, 4.2 Hz, 1H), 6.52 (dd, J=14.3, 8.0 Hz, 1H), 4.93 (dd, J=8.1, 5.4 Hz, 1H), 4.77-4.65 (m, 3H), 4.20-4.05 (m, 2H), 3.45-3.26 (m, 2H), 2.93-2.83 (m, 2H), 2.76-2.60 (m, 2H), 2.34-2.06 (m, 4H), 1.74-1.57 (m, 1H); MS (APCI⁺) m/z 500 (M−H₂O+H)⁺.

### Example 63: (2R)-6-chloro-4-oxo-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide (Compound 162)

### Example 63A: (R)-tert-butyl (3-(6-chloro-4-oxochroman-2-carboxamido)bicyclo[1.1.1]pentan-1-yl)carbamate

The reaction and purification conditions described in Example 2B substituting tert-butyl (3-aminobicyclo[1.1.1]pentan-1-yl)carbamate (PharmaBlock) for the product of Example 2A gave the title compound. MS (ESI⁺) m/z 407 (M+H)⁺.

### Example 63B: (2R)-6-chloro-4-oxo-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide

The reaction and purification conditions described in Example 1C substituting the product of Example 63A for the product of Example 1A and the product of Example 13P for the product of Example 1B gave the title compound. ¹H NMR (400 MHz, DMSO-d₆) δ ppm 8.94 (s, 1H), 8.38 (s, 1H), 7.68-7.60 (m, 2H), 7.21-7.13 (m, 1H), 5.08 (t, J=7.1 Hz, 1H), 4.48 (p, J=7.2 Hz, 1H), 3.72 (s, 2H), 3.76-3.63 (m, 1H), 2.95 (d, J=7.1 Hz, 2H), 2.79-2.67 (m, 2H), 2.23 (s, 6H), 2.20-2.08 (m, 2H); MS (APCI⁺) m/z 503 (M+H)⁺.

### Example 64: 6-chloro-4-oxo-N-(1-5-[cis-3-(trifluoromethoxy)cyclobutyl]-1,3,4-oxadiazol-2-yl)-2-oxabicyclo[2.2.2]octan-4-yl)-3,4-dihydro-2H-1-benzopyran-2-carboxamide (Compound 163)

### Example 64A: Tert-Butyl (1-vinyl-1-oxabicyclo[2.2.2]octan-4-yl)carbamate

To a solution of 1-vinyl-2-oxabicyclo[2.2.2]octane-4-carboxylic acid (4.3 g, 23.60 mmol) in toluene (150 mL) was added 4 Å molecular sieves (6 g, 23.60 mmol), diphenylphosphoryl azide (7.14 g, 26.0 mmol) and triethylamine (3.62 mL, 26.0 mmol) in order at 20° C., and the mixture was stirred under N₂ at 20° C. for 2 hours and then at 120° C. for 2 hours. After insoluble materials were filtered off, the filtrate was concentrated in vacuo. To a solution of the residue in anhydrous tetrahydrofuran (120 mL) was added potassium tert-butoxide (5.83 g, 51.9 mmol) under cooling with ice, the mixture was stirred at 20° C. for 12 hours. After quenching the reaction by addition of 10% aqueous citric acid solution, the mixture was concentrated in vacuo. After dilution of the residue with ethyl acetate, the mixture was washed with saturated sodium hydrogen carbonate solution, water and brine, dried over anhydrous sodium sulfate, filtered, and then concentrated in vacuo. The residue was purified by column chromatography on silica gel eluted with petroleum ether and ethyl acetate from 100:1 to 10:1 to give the title compound (5.2 g, yield 85%). ¹H NMR (400 MHz, CDCl₃) δ ppm 5.81 (dd, J=17.54, 10.96 Hz, 1H), 5.15 (d, J=17.54 Hz, 1H), 5.03 (d, J=10.96 Hz, 1H), 4.29 (br s, 1H), 3.99 (s, 2H), 2.05-2.16 (m, 2H), 1.91-2.02 (m, 2H), 1.74-1.91 (m, 4H), 1.42 (s, 9H).

### Example 64B: Tert-Butyl (1-formyl-2-oxabicyclo[2.2.2]octan-4-yl)carbamate

To a solution of the product of Example 64A (2.6 g, 9.75 mmol) in tetrahydrofuran (90 mL) and water (60 mL) was added sodium periodate (6.26 g, 29.2 mmol) and osmium tetroxide (1.239 g, 4.87 mmol) in order at 0° C. and the mixture was stirred for 12 hours at 20° C. One additional reaction on 2.6 g scale was set up as described above. And these two reactions were combined, diluted with water (200 mL), and extracted with ethyl acetate (2×250 mL). The combined organic fractions were dried with Na₂SO₄ and concentrated under reduced pressure, and the residue was purified by column chromatography on silica gel eluted with petroleum ether:ethyl acetate (100:1 to 4:1) to give the title compound (3.7 g, yield 70.6%). ¹H NMR (400 MHz, CDCl₃) δ ppm 9.57 (s, 1H), 7.26 (s, 1H), 4.33 (br s, 1H), 4.06 (s, 2H), 2.08-2.20 (m, 2H), 1.94-2.05 (m, 2H), 1.81-1.92 (m, 4H), 1.42 (s, 9H).

### Example 64C: Tert-Butyl (1-formyl-2-oxabicyclo[2.2.2]octan-4-yl)carbamate

To a solution of the product of Example 64B (1.9 g, 7.07 mmol) in tetrahydrofuran (60 mL), 2-methylpropan-2-ol (60 mL, 656 mmol) and water (20 mL) was added sodium dihydrogen phosphate (3.39 g, 28.3 mmol) and 2-methyl-2-butene (7.49 mL, 70.7 mmol) and sodium chlorite (1.279 g, 14.14 mmol) in order at 20° C. and the mixture was stirred at 20° C. for 12 hours. One additional reaction on 0.3 g scale and two additional reactions on 0.5 g scale were set up as described above. These four reactions were combined and concentrated under reduced pressure to remove most of volatiles, and the remaining mixture was diluted with water (50 mL), adjusted to pH=12 by aqueous NaOH (1 M), and extracted with methyl tert-butyl ether (50 mL) and ethyl acetate (50 mL) in order. The aqueous layer was adjusted to pH=1 by aqueous HCl (1 M) and extracted with ethyl acetate (2×100 mL). The organic phase was dried with Na₂SO₄ and concentrated under reduced pressure to give the title compound (3.1 g, yield 91%). ¹H NMR (400 MHz, DMSO-d₆) δ ppm 12.42 (br s, 1H), 6.68 (br s, 1H), 3.80 (s, 2H), 1.85-1.99 (m, 5H), 1.85-1.99 (m, 1H), 1.73-1.84 (m, 2H), 1.35 (s, 9H).

### Example 64D: cis-3-(trifluoromethoxy)cyclobutanecarbohydrazide

A mixture of the product of Example 25N (0.8 g, 2.92 mmol) and hydrazine hydrate (1.419 mL, 14.59 mmol) in ethanol (12.0 mL) was heated at reflux for 16 hours. Solvent and excess hydrazine were removed under the high vacuum to give 0.56 g of the title compound. ¹H NMR (400 MHz, DMSO-d₆) δ ppm 9.08 (s, 1H), 4.76 (p, J=7.6 Hz, 1H), 4.22 (s, 2H), 2.60-2.50 (m, 1H), 2.44 (dtd, J=10.1, 7.1, 2.8 Hz, 2H), 2.34-2.18 (m, 2H).

### Example 64E: Tert-Butyl (1-(2-(cis-3-(trifluoromethoxy)cyclobutanecarbonyl)hydrazinecarbonyl)-2-oxabicyclo[2.2.2]octan-4-yl)carbamate

To a mixture of the product of Example 64C (0.2 g, 0.737 mmol), the product of Example 64D (0.153 g, 0.774 mmol), and N-ethyl-N-isopropylpropan-2-amine (0.386 mL, 2.211 mmol) in N,N-dimethylformamide (5.0 mL), 2-(3H-[1,2,3]triazolo[4,5-b]pyridin-3-yl)-1,1,3,3-tetramethylisouronium hexafluorophosphate(V) (0.350 g, 0.921 mmol) was added and the mixture was stirred at ambient temperature for 1 hour. Solvent was removed under high vacuum and the residue was purified by HPLC (Phenomenex® Luna® C18(2) 10 μm 100 Å AXIA™ column (250 mm×50 mm). A 5-80% gradient of acetonitrile (A) and 0.1% trifluoroacetic acid in water (B) was used over 25 minutes, at a flow rate of 50 mL/minute) to give 292 mg of the title compound. ¹H NMR (500 MHz, DMSO-d₆) δ ppm 9.77 (s, 1H), 9.37 (s, 1H), 6.72 (s, 1H), 4.78 (p, J=7.6 Hz, 1H), 3.89 (s, 2H), 2.66 (tt, J=9.4, 7.5 Hz, 1H), 2.47 (dp, J=7.0, 2.4 Hz, 1H), 2.28 (dd, J=8.8, 2.9 Hz, 1H), 2.28-2.17 (m, 1H), 1.99-1.90 (m, 4H), 1.84 (ddt, J=19.0, 14.2, 6.0 Hz, 4H), 1.37 (s, 8H).

### Example 64F: Tert-Butyl (1-(5-(cis-3-(trifluoromethoxy)cyclobutyl)-1,3,4-oxadiazol-2-yl)-2-oxabicyclo[2.2.2]octan-4-yl)carbamate

The title compound was synthesized using the same procedure as described in Example 25Q substituting the product of Example 25P with the product of Example 64E. ¹H NMR (400 MHz, DMSO-d₆) δ ppm 6.80 (s, 1H), 4.90 (p, J=7.5 Hz, 1H), 3.92 (s, 2H), 3.50-3.38 (m, 1H), 2.84 (dtt, J=9.6, 7.4, 2.5 Hz, 2H), 2.50-2.43 (m, 1H), 2.36-2.23 (m, 2H), 2.12-2.00 (m, 2H), 1.93-1.82 (m, 2H), 1.37 (s, 9H); MS (APCI⁺) m/z 433.98 (M+H)⁺.

### Example 64G: 1-(5-(cis-3-(trifluoromethoxy)cyclobutyl)-1,3,4-oxadiazol-2-yl)-2-oxabicyclo[2.2.2]octan-4-amine, Trifluoroacetic Acid

A solution of the product of Example 64F (0.15 g, 0.346 mmol) in dichloromethane (5.0 mL) was treated with 2,2,2-trifluoroacetic acid (1.333 mL, 17.30 mmol). The reaction mixture was stirred at ambient temperature for 2 hours. Solvent and excess 2,2,2-trifluoroacetic acid were removed under high vacuum and the residue was purified by HPLC (Phenomenex® Luna® C18(2) 10 μm 100 Å AXIA™ column (250 mm×50 mm). A 5-80% gradient of acetonitrile (A) and 0.1% trifluoroacetic acid in water (B) was used over 25 minutes, at a flow rate of 50 mL/minute) to give 162 mg of the title compound. ¹H NMR (400 MHz, DMSO-d₆) δ ppm 8.27 (s, 3H), 4.91 (p, J=7.4 Hz, 1H), 3.89 (s, 2H), 3.45 (tt, J=9.8, 7.8 Hz, 1H), 2.84 (dtt, J=9.6, 7.4, 2.5 Hz, 2H), 2.50-2.33 (m, 3H), 2.23-2.10 (m, 2H), 1.99 (tq, J=11.0, 6.8, 6.2 Hz, 4H); MS (APCI⁺) m/z 334.1 (M+H)⁺.

### Example 64H: 6-chloro-4-oxo-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide

The product of Example 64G (0.06 g, 0.134 mmol), 6-chloro-4-oxochroman-2-carboxylic acid (0.033 g, 0.148 mmol), and N-ethyl-N-isopropylpropan-2-amine (0.094 mL, 0.537 mmol) were combined in N,N-dimethylformamide (1.5 mL). 2-(3H-[1,2,3]Triazolo[4,5-b]pyridin-3-yl)-1,1,3,3-tetramethylisouronium hexafluorophosphate(V) (0.064 g, 0.168 mmol) was added and the mixture was stirred at ambient temperature for 1 hour. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 57 mg of the title compound. ¹H NMR (500 MHz, DMSO-d₆) δ ppm 8.04 (s, 1H), 7.67-7.60 (m, 2H), 7.17 (d, J=8.7 Hz, 1H), 5.10 (dd, J=8.5, 4.9 Hz, 1H), 4.90 (p, J=7.5 Hz, 1H), 3.99 (t, J=1.3 Hz, 2H), 3.43 (tt, J=9.9, 7.8 Hz, 1H), 3.03-2.79 (m, 5H), 2.73 (s, 1H), 2.50-2.43 (m, 1H), 2.33 (ddt, J=13.1, 11.2, 3.6 Hz, 2H), 2.17 2.11 (m, 1H), 2.09 (ddd, J=15.8, 12.7, 4.0 Hz, 3H), 1.98 (dd, J=10.3, 6.8 Hz, 2H); MS (APCI⁺) m/z 541.67 (M+H)⁺.

### Example 65: (2R,4R)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide (Compound 164)

The reaction and purification conditions described in Example 6C substituting the product of Example 63B for the product of Example 6B gave the title compound. ¹H NMR (400 MHz, DMSO-d₆) δ ppm 8.65 (s, 1H), 8.35 (s, 1H), 7.37 (dd, J=2.8, 1.0 Hz, 1H), 7.19 (dd, J=8.7, 2.7 Hz, 1H), 6.88 (d, J=8.7 Hz, 1H), 5.70 (br s, 1H), 4.80 (dd, J=10.7, 5.9 Hz, 1H), 4.59 (dd, J=12.0, 2.2 Hz, 1H), 4.48 (p, J=7.2 Hz, 1H), 3.73 (s, 2H), 3.72-3.66 (m, 1H), 2.79-2.68 (m, 2H), 2.39-2.30 (m, 1H), 2.26 (s, 6H), 2.20-2.09 (m, 2H), 1.75-1.62 (m, 1H); MS (APCI⁺) m/z 487 (M−H₂O+H)⁺.

### Example 66: 6-chloro-4-hydroxy-N-(1-5-[cis-3-(trifluoromethoxy)cyclobutyl]-1,3,4-oxadiazol-2-yl)-2-oxabicyclo[2.2.2]octan-4-yl)-3,4-dihydro-2H-1-benzopyran-2-carboxamide (Compound 165)

To a suspension of the product of Example 64 (0.043 g, 0.079 mmol) in methanol (2.0 mL), sodium tetrahydroborate (6.00 mg, 0.159 mmol) was added and the reaction mixture was stirred at ambient temperature for 15 minutes. Solvent was removed under vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 32 mg of the title compound. ¹H NMR (500 MHz, DMSO-d₆) δ ppm 7.66 (s, 1H), 7.38 (d, J=2.7 Hz, 1H), 7.19 (dd, J=8.7, 2.7 Hz, 1H), 6.87 (d, J=8.7 Hz, 1H), 4.90 (p, J=7.5 Hz, 1H), 4.79 (dd, J=10.6, 5.9 Hz, 1H), 4.60 (dd, J=11.7, 2.3 Hz, 1H), 4.10-4.00 (m, 2H), 3.44 (dd, J=17.7, 2.1 Hz, 1H), 2.85 (dtt, J=9.7, 7.3, 2.4 Hz, 2H), 2.55 (d, J=13.7 Hz, 1H), 2.51-2.44 (m, 1H), 2.41-1.98 (m, 10H); MS (APCI⁺) m/z 544.15 (M+H)⁺.

### Example 67: 2-(4-chloro-3-fluorophenoxy)-N-(3-{5-[rac-(2R,4R)-6-chloro-4-hydroxy-3,4-dihydro-2H-1-benzopyran-2-yl]-1,3,4-oxadiazol-2-yl}bicyclo[1.1.1]pentan-1-yl)acetamide (Compound 166)

### Example 67A: Methyl rac-(2R,4R)-4-{[tert-butyl(dimethyl)silyl]oxy}-6-chloro-3,4-dihydro-2H-1-benzopyran-2-carboxylate

To a solution of methyl 6-chloro-4-hydroxychroman-2-carboxylate (Princeton, 1.49 g, 6.12 mmol) in tetrahydrofuran (24 mL) at 0° C. and was added tert-butyldimethylchlorosilane (TBS-Cl, 2.03 g, 13.5 mmol) followed by imidazole (1.00 g, 14.7 mmol). The cooling bath was removed and the flask was allowed to warm to ambient temperature overnight. The reaction mixture was diluted with water (80 mL), extracted with diethyl ether (3×25 mL), and concentrated in vacuo. A portion of the residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title intermediate. ¹H NMR (500 MHz, DMSO-d₆) δ ppm 7.25 (dd, J=8.7, 2.6 Hz, 1H), 7.16 (dd, J=2.7, 0.7 Hz, 1H), 6.91 (d, J=8.8 Hz, 1H), 5.07 (dd, J=6.5, 4.6 Hz, 1H), 4.97-4.92 (m, 1H), 3.66 (s, 3H), 2.35 (dt, 0.1=13.9, 4.6 Hz, 1H), 2.15 (dt, J=13.9, 6.2 Hz, 1H), 0.87 (s, 9H), 0.16 (s, 3H), 0.15 (s, 3H).

### Example 67B: rac-(2R,4R)-4-{[tert-butyl(dimethyl)silyl]oxy}-6-chloro-3,4-dihydro-2H-1-benzopyran-2-carbohydrazide

The methodologies described in Example 51A substituting Example 67A for Example 25N and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title intermediate. MS (APCI⁺) m/z 357 (M+H)⁺.

### Example 67C: 2-(4-chloro-3-fluorophenoxy)-N-(3-{2-[rac-(2R,4R)-6-chloro-4-hydroxy-3,4-dihydro-2H-1-benzopyran-2-carbonyl]hydrazinecarbonyl}bicyclo[1.1.1]pentan-1-yl)acetamide

The methodologies described in Example 30D substituting Example 67B for Example 30C and substituting Hunig's base (1.7 equivalents) for triethylamine gave the title intermediate. ¹H NMR (600 MHz, DMSO-d₆) δ ppm 10.00 (d, J=1.3 Hz, 1H), 9.81 (d, J=1.2 Hz, 1H), 8.76 (s, 1H), 7.50 (t, J=8.9 Hz, 1H), 7.39 (dd, J=2.8, 1.0 Hz, 1H), 7.23-7.15 (m, 1H), 7.11-7.05 (m, 1H), 6.88-6.84 (m, 2H), 4.84 (dd, J=10.7, 5.8 Hz, 1H), 4.79 (dd, J=12.1, 2.3 Hz, 1H), 4.48 (s, 2H), 2.41-2.34 (m, 1H), 2.25 (s, 6H), 2.22 (dd, J=13.6, 5.5 Hz, 1H), 1.75 (td, J=12.5, 10.8 Hz, 1H); MS (APCI⁺) m/z 521 (M−H₂O+H)⁺.

### Example 67D: 2-(4-chloro-3-fluorophenoxy)-N-{3-[5-(6-chloro-4-hydroxy-3,4-dihydro-2H-1-benzopyran-2-yl)-1,3,4-oxadiazol-2-yl]bicyclo[1.1.1]pentan-1-yl}acetamide

The methodologies described in Example 25Q substituting Example 67C for Example 25 P gave the title compound. ¹H NMR (600 MHz, DMSO-d₆, dr 25:1) δ ppm 8.95 (s, 1H), 7.51 (t, J=8.9 Hz, 1H), 7.43 (dd, J=2.7, 1.0 Hz, 1H), 7.39 (t, J=3.2 Hz, 0.07H), 7.27 (dd, J=8.8, 2.7 Hz, 0.05H), 7.21 (ddd, J=8.8, 2.7, 0.7 Hz, 1H), 7.09 (dd, J=11.3, 2.8 Hz, 1H), 7.03 (s, 0.06H), 6.93 (d, J=8.8 Hz, 0.06H), 6.87 (ddd, J=9.0, 2.8, 1.2 Hz, 1H), 6.86 (d, J=8.7 Hz, 1H), 5.81 (s, 1H), 5.69 (dd, J=11.5, 2.3 Hz, 1H), 5.58-5.53 (m, 0.04H), 4.91 (dd, J=10.3, 5.9 Hz, 1H), 4.75 (s, 0.03H), 4.51 (s, 2H), 2.56-2.51 (m, 1H), 2.51 (s, 6H), 2.32-2.30 (m, 0.09H), 2.15 (ddd, J=13.1, 11.6, 10.4 Hz, 1H); MS (APCI⁺) m/z 502 (M−H₂O+H)⁺.

### Example 68: 6-chloro-4-oxo-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide (Compound 167)

### Example 68A: Tert-Butyl (4-(2-(cis-3-(trifluoromethoxy)cyclobutanecarbonyl)hydrazinecarbonyl)bicyclo[2.2.2]octan-1-yl)carbamate

The methodologies described in Example 25P substituting Example 51A for Example 25L and substituting 4-(tert-butoxycarbonyl)amino)bicycle[2.2.2]octane-1-carboxyl acid (purchased from AChemBlock) for Example 250 gave the title intermediate. MS (APCI⁺) m/z 450 (M+H)⁺.

### Example 68B: Tert-Butyl (4-(5-((cis)-3-(trifluoromethoxy)cyclobutyl)-1,3,4-oxadiazol-2-yl)bicyclo[2.2.2]octan-1-yl)carbamate

The methodologies described in Example 25Q substituting Example 68A for Example 25P gave the title intermediate. ¹H NMR (400 MHz, CDCl₃) δ ppm 4.73-4.62 (m, 1H), 4.40-4.35 (m, 1H), 2.87-2.78 (m, 2H), 2.64 (q, J=10.0 Hz, 2H), 2.09-2.01 (m, 6H), 2.00-1.92 (m, 6H), 1.43 (s, 9H); MS (APCI⁺) m/z 432 (M+H)⁺.

### Example 68C: 4-(5-((cis)-3-(trifluoromethoxy)cyclobutyl)-1,3,4-oxadiazol-2-yl)bicyclo[2.2.2]octan-1-amine

The methodologies described in Example 21B substituting Example 68B for Example 21A gave the title intermediate. MS (APCI⁺) m/z 332 (M+H)⁺.

### Example 68D: 6-chloro-4-oxo-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide

The methodologies described in Example 30D substituting 6-chloro-4-oxochroman-2-carboxylic acid (Princeton Bio) for 3-(2-(4-chloro-3-fluorophenoxy)acetamido)bicyclo[1.1.1]pentane-1-carboxylic acid and substituting Example 68C for Example 30C gave the title compound. ¹H NMR (500 MHz, CDCl₃) δ ppm 7.89 (d, J=2.7 Hz, 1H), 7.48 (dd, J=8.8, 2.7 Hz, 1H), 7.03 (d, J=8.8 Hz, 1H), 6.31 (s, 1H), 4.80 (dd, J=13.0, 3.4 Hz, 1H), 4.75-4.65 (m, 1H), 3.32 (tt, J=10.1, 7.7 Hz, 1H), 3.17 (dd, J=17.3, 3.4 Hz, 1H), 2.90-2.80 (m, 3H), 2.65 (tdd, J=10.1, 7.8, 2.9 Hz, 2H), 2.10 (s, 12H); MS (APCI⁺) m/z 540 (M+H)⁺.

### Example 69: 2-(4-chloro-3-fluorophenoxy)-N-[rac-(1R,2S,4R,5S)-5-{5-[cis-3-(trifluoromethoxy)cyclobutyl]-1,3,4-oxadiazol-2-yl}-7-oxabicyclo[2.2.1]heptan-2-yl]acetamide (Compound 168)

### Example 69A: furan-3-ylmethanol

To a solution of furan-3-carboxylic acid (50 g, 446 mmol) in tetrahydrofuran (500 mL) was added a solution of borane in tetrahydrofuran (669 mL, 669 mmol) at 0° C., and the mixture was stirred at 20° C. for 1 hour. One additional vial on 25 g scale and six additional vials on 50 g scale were set up as described above. The reactions conducted in parallel were combined for work up. After cooling to 0° C., the reaction mixture was quenched with water until gas evolution had ceased. After bulk solvent removal, the resulting crude residue was then partitioned between saturated aqueous NaHCO₃ and ethyl acetate, and the aqueous layer was further extracted with ethyl acetate (2×1000 mL). The combined organic phases were washed with brine (1000 mL), dried Na₂SO₄, and concentrated to dryness under reduced pressure. The residue was purified by column chromatography on silica gel with petroleum ether:ethyl acetate=3:1 to give the title compound (230 g, yield 63.1%) as a yellow oil. ¹HNMR (400 MHz, DMSO-d₆) δ ppm 7.46-7.61 (m, 2H), 4.34 (d, J=5.50 Hz, 2H), 4.97 (t, J=5.50 Hz, 1H), 6.44 (d, J=0.63 Hz, 1H).

### Example 69B: 3-((benzyloxy)methyl)furan

To a solution of the product of Example 69A (20 g, 183 mmol) in N,N-dimethylformamide (200 mL) was added NaH (8.81 g, 220 mmol) at 0° C. and the mixture was stirred at 0° C. for 0.5 hour. (Bromomethyl)benzene (37.7 g, 220 mmol) was added at 0° C. and the reaction mixture was stirred at 20° C. for 12 hours. One additional vial on 5 g scale and nine additional vials on 20 g scale were set up as described above. The reactions conducted in parallel were combined for work up. After cooling to 0° C., the reaction was quenched with water until gas evolution ceased. The mixture was extracted with ethyl acetate (3×3000 mL). The combined organic fractions were washed with brine (2×1000 mL), dried over Na₂SO₄, filtered and concentrated under reduced pressure. The residue was purified by column chromatography on silica gel eluted with petroleum ether:ethyl acetate=100:1 to 50:1 to give the title compound (480 g, yield 91%). ¹H NMR (400 MHz, CDCl₃) δ ppm 7.27 (s, 7H), 6.37 (s, 1H), 4.45 (s, 2H), 4.35 (s, 2H).

### Example 69C: rac-(1R,2R,4R)-5-((benzyloxy)methyl)-7-oxabicyclo[2.2.1]hept-5-ene-2-carbonitrile

Acrylonitrile (33.8 g, 638 mmol) was treated portion-wise with zinc chloride (20.85 g, 153 mmol), and the mixture was stirred at 20° C. for 10 minutes. Then the product of Example 69B (30 g, 128 mmol) was added to the mixture and the mixture was stirred at 20° C. for 12 hours. Fifteen additional vials on 30 g scale were set up as described above. The reactions conducted in parallel were combined for work up. The combined reaction mixtures were diluted with ethyl acetate (1000 mL) and purified by column chromatography on silica gel eluted with ethyl acetate:petroleum ether (1:3) to give the title compound (129 g, yield 20.96%). ¹H NMR (400 MHz, DMSO-d₆) δ ppm 7.20-7.41 (m, 5H), 6.01-6.33 (m, 1H), 5.17-5.23 (m, 1H), 5.01-5.08 (m, 1H), 4.40-4.52 (m, 2H), 4.08-4.23 (m, 2H), 3.97-4.07 (m, 1H), 2.72 (dd, J=8.57, 3.81 Hz, 1H), 1.98 (s, 2H), 1.85-1.94 (m, 1H), 1.71-1.82 (m, 1H), 1.17 (t, J=7.13 Hz, 2H).

### Example 69D: rac-(1R,2R,4R)-5-((benzyloxy)methyl)-7-oxabicyclo[2.2.1]heptane-2-carbonitrile

To a solution of the product of Example 69C (15 g, 49.7 mmol) in methanol (150 mL) was added Pd/C (5.29 g, 2.487 mmol) under argon, and the mixture was stirred at 20° C. under hydrogen (15 psi) for 2 hours. One additional vial on 1 g scale and two additional vials on 15 g scale were set up as described above. The reactions conducted in parallel were combined for work up. The suspension was filtered through a pad of diatomaceous earth and the pad was washed with methanol (5×200 mL). The combined filtrates were concentrated to dryness and the residue was purified by column chromatography on silica gel eluted with petroleum ether:ethyl acetate (3:1) to give the title compound (38 g, yield 64.5%). ¹H NMR (400 MHz, DMSO-d₆) δ ppm 7.25-7.45 (m, 5H), 4.74-4.88 (m, 1H), 4.56-4.71 (m, 1H), 4.37-4.52 (m, 1H), 3.45-3.64 (m, 1H), 2.89-3.23 (m, 1H), 2.09-2.36 (m, 2H), 1.85-2.04 (m, 1H), 1.62-1.84 (m, 1H), 1.05 (dd, 0.1=12.51, 5.50 Hz, 1H).

### Example 69E: rac-(1R,2S,4R)-5-((benzyloxy)methyl)-7-oxabicyclo[2.21.]heptane-2-carboxylic Acid

To a solution of the product of Example 69D (27 g, 89 mmol) in ethanol (270 mL) was added and aqueous solution of 3 N KOH (237 mL, 710 mmol) at 20° C., and the mixture was stirred at 100° C. for 16 hours. One additional vial on 1 g scale and one additional vial on 10 g scale were set up as described above. The reactions conducted in parallel were combined for work up. The mixture was concentrated under reduced pressure, and the residue was extracted with ethyl acetate (3-500 mL). The aqueous phase was adjusted to pH=1 with 1 N HCl. The mixture was extracted with ethyl acetate (3×500 mL), and the combined organic fractions were concentrated under reduced pressure to give the title compound (35 g, yield 85%). ¹H NMR (400 MHz, DMSO-d₆) δ ppm 12.03-12.41 (m, 1H), 7.23-7.49 (m, 5H), 4.55-4.67 (m, 1H), 4.33-4.54 (m, 3H), 3.52 (dd, J=9.66, 6.36 Hz, 1H), 2.19-2.38 (m, 1H), 1.70-1.90 (m, 2H), 1.02 (dd, J=12.04, 5.20 Hz, 1H).

### Example 69F: 2-(trimethylsilyl)ethyl (rac-(1S,2R,4S)-5-((benzyloxy)methyl)-7-oxabicyclo[2.2.1]heptan-2-yl)carbamate

To a mixture of the product of Example 69E (6.0 g, 22.87 mmol), N,N-diisopropylethylamine (11.99 mL, 68.6 mmol) and 2-(trimethylsilyl)ethanol (21.0 g, 178 mmol) in toluene (60 mL) stirred at ambient temperature was added diphenylphosphoryl azide (7.9 mL, 0.00 mmol). The resulting solution was heated at 80° C. for 16 hours and cooled down to ambient temperature. The reaction mixture was diluted with toluene (30 mL) and washed with water (50 mL), saturated sodium bicarbonate solution (50 mL) and then brine (50 mL). The organic phase was dried with magnesium sulfate, filtered and concentrated. The residue was purified on silica gel using a gradient of 0-40% ethyl acetate in heptane to give 5.72 g of the title compound. ¹H NMR (500 MHz, DMSO-d₆) δ ppm 7.38-7.23 (m, 5H), 7.03 (d, J=6.3 Hz, 1H), 4.50-4.35 (m, 3H), 4.14 (d, J=5.9 Hz, 1H), 4.06-3.97 (m, 2H), 3.52-3.41 (m, 2H), 3.31-3.25 (m, 2H), 2.19 (tdd, J=11.4, 7.6, 4.7 Hz, 1H), 2.04-1.96 (m, 1H), 1.82-1.65 (m, 1H), 0.90-0.81 (m, 3H), 0.00 (s, 9H).

### Example 69G: 2-(trimethylsilyl)ethyl (rac-(1R,2S,4R)-5-(hydroxymethyl)-7-oxabicyclo[2.2.1]heptan-2-yl)carbamate

To a solution of the product of Example 69F (5.72 g, 15.15 mmol) in tetrahydrofuran (69 mL) was added 10% Pd(OH)₂/C (2.8 g, 9.97 mmol) in a 160 mL stainless steel reactor. The suspension was stirred under 60 psi of hydrogen at ambient temperature for 18 hours. The mixture was filtered and the filtrate was concentrated to give 4.08 g of the title compound, used without further purification. ¹H NMR (400 MHz, DMSO-d₆) δ ppm 6.99 (d, J=6.3 Hz, 1H), 4.51 (t, J=4.9 Hz, 1H), 4.35 (t, J=5.1 Hz, 1H), 4.12 (d, J=5.9 Hz, 1H), 4.10-3.93 (m, 2H), 3.51-3.37 (m, 1H), 3.24 (td, J=10.4, 4.9 Hz, 1H), 2.06 (td, J=12.0, 11.1, 6.7 Hz, 2H), 1.82-1.72 (m, 1H), 1.65 (dt, J=11.8, 5.9 Hz, 1H), 1.38 (d, J=13.4 Hz, 1H), 0.99-0.76 (m, 3H), 0.00 (s, 9H).

### Example 69H: 2-(trimethylsilyl)ethyl (rac-(1R,2S,4R)-5-cyano-7-oxabicyclo[2.2.1]heptan-2-yl)carbamate

To a solution of the product of Example 69G (4.08 g, 14.19 mmol) in acetonitrile/water (9:1, 50 mL) were successively added TEMPO (0.222 g, 1.419 mmol) and ammonium acetate (4.38 g, 56.8 mmol) and (diacetoxyiodo)benzene (10.06 g, 31.2 mmol). The mixture was stirred at ambient temperature for 3 hours. Solvent was removed and the residue was partitioned between water and ethyl acetate. The organic layer was separated, dried over magnesium sulfate, filtered, and concentrated. The residue was purified on silica gel using a gradient of 0-70% ethyl acetate in heptane to give 3.7 g of the title compound. ¹H NMR (400 MHz, DMSO-d₆) δ ppm 7.20-7.08 (m, 1H), 4.79-4.68 (m, 1H), 4.36 (d, J=5.6 Hz, 1H), 4.06-3.96 (m, 2H), 3.64 (ddd, J=8.1, 6.2, 3.4 Hz, 1H), 3.30 (s, 1H), 3.09-2.88 (m, 1H), 2.18 (dd, J=13.5, 8.1 Hz, 1H), 2.11-1.82 (m, 1H), 1.60 (dt, J=12.1, 4.6 Hz, 2H), 0.94-0.85 (m, 2H), 0.00 (s, 9H).

### Example 69I: rac-(1R,2S,4R,5S)-5-(((2-(trimethylsilyl)ethoxy)carbonyl)amino)-7-oxabicyclo[2.2.1]heptane-2-carboxylic Acid

A solution of the product of Example 69H (3.7 g, 13.10 mmol) and potassium hydroxide (43.7 mL, 131 mmol) in ethanol (40 mL) was heated at 80° C. for 5 hours. Solvent was removed and the residue was partitioned between ethyl acetate and water. The aqueous phase was then acidified with cold 0.5 N HCl, and extracted with ethyl acetate. The organic layer was washed with brine, dried over magnesium sulfate, filtered and concentrated to give 0.56 g of the title compound. ¹H NMR (400 MHz, DMSO-d₆) δ ppm 12.18 (s, 1H), 7.03 (d, J=6.4 Hz, 1H), 4.62 (d, J=5.5 Hz, 1H), 4.25 (d, J=5.7 Hz, 1H), 4.08-3.91 (m, 2H), 3.52 (dd, J=8.4, 6.1 Hz, 1H), 2.54 (dd, J=9.1, 4.5 Hz, 1H), 1.93-1.72 (m, 2H), 1.59 (dd, J=12.4, 9.1 Hz, 1H), 1.49 (dt, J=12.6, 4.2 Hz, 1H), 0.98-0.81 (m, 2H), 0.00 (s, 9H).

### Example 69J: 2-(trimethylsilyl)ethyl [rac-(1R,2S,4R,5S)-5-{2-[cis-3-(trifluoromethoxy)cyclobutane-1-carbonyl]hydrazinecarbonyl}-7-oxabicyclo[2.2.1]heptan-2-yl]carbamate

The title compound was synthesized using the procedure described in Example 64E substituting the product of Example 64C with the product of Example 69I. ¹H NMR (400 MHz, DMSO-d₆) δ ppm 9.75 (s, 2H), 7.02 (d, J=6.4 Hz, 1H), 4.77 (p, J=7.4 Hz, 1H), 4.53 (d, J=5.4 Hz, 1H), 4.23 (d, J=5.6 Hz, 1H), 4.01 (t, J=8.8 Hz, 2H), 3.64-3.46 (m, 2H), 3.32 (s, 6H), 2.73-2.60 (m, 1H), 2.48-2.42 (m, 1H), 2.25 (q, J=9.6 Hz, 2H), 1.94-1.81 (m, 2H), 1.60-1.44 (m, 2H), 0.89 (t, J=8.4 Hz, 2H), 0.00 (d, J=1.0 Hz, 9H).

### Example 69K: 2-(4-chloro-3-fluorophenoxy)-N-[rac-(1R,2S,4R,5S)-5-{5-[cis-3-(trifluoromethoxy)cyclobutyl]-1,3,4-oxadiazol-2-yl}-7-oxabicyclo[2.2.1]heptan-2-yl]acetamide

A suspension of the product of Example 69J (0.105 g, 0.218 mmol) in acetonitrile (5.0 mL) was treated with N-ethyl-N-isopropylpropan-2-amine (0.114 mL, 0.654 mmol) and 4-methylbenzene-1-sulfonyl chloride (0.083 g, 0.436 mmol). The reaction mixture was stirred at 50° C. overnight. The mixture was concentrated and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 8 mg of 2-(trimethylsilyl)ethyl [rac-(1R,2S,4R,5S)-5-{5-[cis-3-(trifluoromethoxy)cyclobutyl]-1,3,4-oxadiazol-2-yl}-7-oxabicyclo[2.2.1]heptan-2-yl]carbamate. This intermediate was dissolved in dichloromethane (1.0 mL) and treated with 2,2,2-trifluoroacetic acid (0.5 mL, 6.49 mmol) at ambient temperature for 45 minutes. Solvent and excess 2,2,2-trifluoroacetic acid were removed under high vacuum to give rac-(1R,2S,4R,5S)-5-{5-[cis-3-(trifluoromethoxy)cyclobutyl]-1,3,4-oxadiazol-2-yl}-7-oxabicyclo[2.2.1]heptan-2-amine that was used without further purification. To a mixture of the crude amine, 2-(4-chloro-3-fluorophenoxy)acetic acid (4.41 mg, 0.022 mmol) and N-ethyl-N-isopropylpropan-2-amine (0.030 mL, 0.173 mmol) in N,N-dimethylformamide (1 mL) was added 2-(3H-[1,2,3]triazolo[4,5-b]pyridin-3-yl)-1,1,3,3-tetramethylisouronium hexafluorophosphate(V) (9.84 mg, 0.026 mmol). The reaction mixture was stirred at ambient temperature for 30 minutes. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 5 mg of the title compound. ¹H NMR (400 MHz, CDCl₃) δ ppm 7.33 (t, J=8.6 Hz, 1H), 6.75 (dd, J=10.3, 2.8 Hz, 1H), 6.75-6.60 (m, 2H), 4.91 (d, J=5.6 Hz, 1H), 4.69 (td, J=14.3, 13.5, 6.7 Hz, 1H), 4.59 (d, J=5.8 Hz, 1H), 4.45 (d, J=1.6 Hz, 2H), 4.42-4.30 (m, 1H), 3.39-3.25 (m, 1H), 3.21 (dd, J=9.0, 4.3 Hz, 1H), 2.85 (tdd, J=12.1, 5.9, 1.6 Hz, 2H), 2.67 (td, J=12.5, 11.5, 8.8 Hz, 2H), 2.36 (dt, J=13.2, 5.0 Hz, 1H), 2.30-2.16 (m, 1H), 2.14-1.97 (m, 1H), 1.67 (ddd, J=13.3, 5.8, 3.2 Hz, 2H) MS (APCI⁺) m/z 506.02 (M+H)⁺.

### Example 70: (2R,4R)-6-chloro-4-hydroxy-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide (Compound 169)

Example 62 was purified by chiral SFC (supercritical fluid chromatography) using a (S,S) Whelk-O®1 column (20×250 mm, 5 micron) eluted with 40% CH₃OH in CO₂ at 34° C. with a CO₂ flow rate of 36 mL/minute, CH₃OH flow rate of 24 mL/minute, front pressure of 171 bar, and back pressure of 100 bar to give the title compound (first isomer eluted out of the column, 0.0093 g, 0.018 mmol, 200% yield). The absolute stereochemistry of this title compound was arbitrarily assigned. ¹H NMR (400 MHz, CDCl₃) δ ppm 7.45 (d, J=2.5 Hz, 1H), 7.20 (dd, J=8.7, 2.5 Hz, 1H), 6.87 (d, J=8.8 Hz, 1H), 6.52-6.46 (m, 1H), 5.00-4.87 (m, 2H), 4.75-4.64 (m, 2H), 4.24-4.01 (m, 1H), 3.47-3.28 (m, 1H), 2.91-2.82 (m, 3H), 2.70 (dt, J=20.5, 9.2 Hz, 3H), 2.18 (dd, J=18.4, 6.1 Hz, 3H), 2.10 (d, J=5.1 Hz, 1H); MS (APCI⁺) m/z 500 (M−H₂O+H)⁺.

### Example 71: (2S,4S)-6-chloro-4-hydroxy-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide (Compound 170)

Example 62 was purified by chiral SFC (supercritical fluid chromatography) using a (S,S) Whelk-O®1 column (20×250 mm, 5 micron) eluted with 40% CH₃OH in CO₂ at 34° C. with a CO₂ flow rate of 36 mL/minute, CH₃OH flow rate of 24 mL/minute, front pressure of 171 bar, and back pressure of 100 bar to give the title compound (second isomer eluted out of the column, 0.014 g, 0.028 mmol, 31% yield). The absolute stereochemistry of this title compound was arbitrarily assigned. ¹H NMR (400 MHz, CDCl₃) δ ppm 7.45 (d, J=2.7 Hz, 1H), 7.20 (dd, J=8.7, 2.6 Hz, 1H), 6.86 (d, J=8.7 Hz, 1H), 6.45 (d, J=8.0 Hz, 1H), 4.96-4.89 (m, 1H), 4.73-4.65 (m, 2H), 4.13-4.05 (m, 3H), 3.38-3.27 (m, 1H), 2.86 (dd, J=11.7, 7.1 Hz, 2H), 2.74-2.63 (m, 2H), 2.37-2.25 (m, 1H), 2.24-2.14 (m, 1H), 2.11 (d, J=6.6 Hz, 1H), 1.74-1.64 (m, 1H); MS (APCI⁺) m/z 500 (M−H₂O+H)⁺.

### Example 72: rac-(2R,4R)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide (Compound 171)

The methodologies described in Example 5 substituting Example 68 for Example 4 gave the title compound. ¹H NMR (500 MHz, CDCl₃, dr 33:1) δ ppm 7.44 (dd, J=2.7, 0.8 Hz, 1H), 7.33 (s, 0.01H), 7.18 (dd, J=8.7, 2.6 Hz, 1H), 6.88 (d, J=8.8 Hz, 0.03H), 6.83 (d, J=8.7 Hz, 1H), 6.39 (s, 0.03H), 6.30 (s, 1H), 4.90 (dd, J=7.9, 5.5 Hz, 1H), 4.79 (d, J=3.5 Hz, 0.02H), 4.69 (p, J=7.7 Hz, 1H), 4.58 (dd, J=8.8, 3.4 Hz, 1H), 3.31 (tt, J=10.1, 7.7 Hz, 1H), 2.84 (dtt, J=9.9, 7.3, 2.6 Hz, 2H), 2.69-2.63 (m, 1H), 2.66-2.57 (m, 2H), 2.18 (ddd, J=13.7, 8.8, 7.9 Hz, 1H), 2.12-2.00 (m, 12H); MS (APCI⁺) m/z 542 (M+H)⁺.

### Example 73: (2S,4R)-6-chloro-4-hydroxy-N-[trans-4-({[5-(trifluoromethyl)pyridin-2-yl]methyl}carbamoyl)cyclohexyl]-3,4-dihydro-2H-1-benzopyran-2-carboxamide (Compound 172)

### Example 73A: (2S,4S)-6-chloro-4-hydroxychroman-2-carboxylic Acid

The reaction and purification conditions described in Example 3B substituting the product of Example 10A for the product of Example 1B gave the title compound. MS (APCI⁻) m/z 227 (M−H)⁻.

### Example 73B: (2S,4R)-6-chloro-4-hydroxychromane-2-carboxylic Acid

The product of Example 73A (140 mg, 0.612 mmol) was combined with trifluoroacetic acid (1.0 mL) and stirred at 30° C. for 2 hours. The reaction mixture was concentrated under high vacuum. The residue was taken up in acetonitrile (3.0 mL), and aqueous ammonium hydroxide (3 M, 3 mL) was added. The resulting mixture was stirred at ambient temperature for 18 hours and then concentrated under high vacuum. The residue was taken up in methanol, filtered through a glass microfiber frit and purified by preparative HPLC [Waters SunFire™ C18 5 μm OBD column, 30×150 mm, flow rate 30 mL/minute, 3-100% gradient of acetonitrile in buffer (0.1% trifluoroacetic acid)] to give the title compound (80 mg, 0.35 mmol, 57% yield). MS (ESI⁻) m/z 227 (M−H)⁻.

### Example 73C: (2S,4R)-6-chloro-4-hydroxy-N-[trans-4-({[5-(trifluoromethyl)pyridin-2-yl]methyl}carbamoyl)cyclohexyl]-3,4-dihydro-2H-1-benzopyran-2-carboxamide

The reaction and purification conditions described in Example 3C substituting the product of Example 59A for the product of Example 3A, and the product of Example 73B for the product of Example 3B gave the title compound. ¹H NMR (400 MHz, DMSO-d₆) δ ppm 8.90-8.85 (m, 1H), 8.48 (t, J=5.9 Hz, 1H), 8.17 (dd, J=8.3, 2.4 Hz, 1H), 7.95 (d, J=8.2 Hz, 1H), 7.45 (d, J=8.3 Hz, 1H), 7.31 (d, J=2.7 Hz, 1H), 7.24 (dd, J=8.7, 2.7 Hz, 1H), 6.93 (d, J=8.7 Hz, 1H), 5.62 (br s, 1H), 4.62-4.53 (m, 2H), 4.43 (d, J=5.2 Hz, 2H), 3.58 (s, 1H), 2.19 (tt, =11.8, 3.2 Hz, 1H), 2.09 (dt, J=13.9, 3.4 Hz, 1H), 1.97-1.76 (m, 5H), 1.52-1.25 (m, 4H), MS (APCI⁺) m/z 512 (M+H)⁺.

### Example 74: (2R)-6-chloro-N-{trans-4-[3-(4-chlorophenyl)azetidine-1-carbonyl]cyclohexyl}-4-oxo-3,4-dihydro-2H-1-benzopyran-2-carboxamide (Compound 173)

### Example 74A: Tert-Butyl (trans-4-(3-(4-chlorophenyl)azetidine-1-carbonyl)cyclohexyl)carbamate

The reaction and purification conditions described in Example 2B substituting 3-(4-chlorophenyl)azetidine (Enamine) for the product of Example 2A, and trans-4-((tert-butoxycarbonyl)amino)cyclohexanecarboxylic acid (Ark Pharm) for the product of Example 1B gave the title compound. MS (APCI⁺) m/z 393 (M+H)⁺.

### Example 74B: (2R)-6-chloro-N-{trans-4-[3-(4-chlorophenyl)azetidine-1-carbonyl]cyclohexyl}-4-oxo-3,4-dihydro-2H-1-benzopyran-2-carboxamide

The reaction and purification conditions described in Example 1C substituting the product of Example 74A for the product of Example 1A give the title compound. ¹H NMR (400 MHz, DMSO-d₆) δ ppm 8.14 (d, J=7.9 Hz, 1H), 7.67-7.59 (m, 2H), 7.45-7.35 (m, 4H), 7.16 (d, J=8.7 Hz, 1H), 5.11 (dd, J=7.8, 5.5 Hz, 1H), 4.55 (t, J=8.5 Hz, 1H), 4.22 (t, J=8.9 Hz, 1H), 4.14 (dd, J=8.5, 5.9 Hz, 1H), 3.90-3.79 (m, 1H), 3.77 (dd, J=9.2, 6.2 Hz, 1H), 3.54-3.46 (m, 1H), 3.04-2.88 (m, 2H), 2.22-2.11 (m, 1H), 1.86-1.65 (m, 4H), 1.43-1.16 (m, 4H) MS (APCI⁺) m/z 501 (M+H)⁺.

### Example 75: (2R,4R)-6-chloro-N-{trans-4-[3-(4-chlorophenyl)azetidine-1-carbonyl]cyclohexyl}-4-hydroxy-3,4-dihydro-2H-1-benzopyran-2-carboxamide (Compound 174)

The reaction and purification conditions described in Example 6C substituting the product of Example 74B for the product of Example 6B gave the title compound. ¹H NMR (400 MHz, DMSO-d₆) δ ppm 7.87 (d, J=8.1 Hz, 1H), 7.46-7.35 (m, 5H), 7.19 (dd, J=8.7, 2.7 Hz, 1H), 6.88 (d, J=8.7 Hz, 1H), 5.69 (br s, 1H), 4.81 (dd, J=10.7, 5.9 Hz, 1H), 4.61 (dd, J=11.9, 2.2 Hz, 1H), 4.56 (t, J=8.5 Hz, 1H), 4.23 (t, J=8.9 Hz, 1H), 4.15 (dd, J=8.5, 5.9 Hz, 1H), 3.91-3.79 (m, 1H), 3.78 (dd, J=9.3, 6.1 Hz, 1H), 3.64-3.50 (m, 1H), 2.34 (ddd, J=12.9, 5.9, 2.3 Hz, 1H), 2.22-2.12 (m, 1H), 1.87-1.66 (m, 5H), 1.48-1.27 (m, 4H); MS (APCI⁺) m/z 503 (M+H)⁺.

### Example 76: (2S)-6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide (Compound 175)

The methodologies described in Example 30D substituting the product of Example 10A for 3-(2-(4-chloro-3-fluorophenoxy)acetamido)bicyclo[1.1.1]pentane-1-carboxylic acid and substituting Example 49B for Example 30C gave the title compound. ¹H NMR (400 MHz, DMSO-d₆) δ ppm 9.19 (s, 1H), 8.47-8.42 (m, 1H), 8.07 (d, J=1.4 Hz, 1H), 7.80 (ddd, J=12.0, 7.7, 2.2 Hz, 1H), 7.70-7.61 (m, 2H), 7.65-7.57 (m, 1H), 7.52 (dt, J=10.7, 8.5 Hz, 1H), 7.23-7.15 (m, 1H), 5.17 (dd, J=8.3, 6.0 Hz, 1H), 3.07-2.92 (m, 2H), 2.57 (s, 6H); MS (APCI⁺) m/z 470 (M+H)⁺.

### Example 77: (2R)-6-chloro-N-{3-[4-(3,4-difluorophenyl)-1H-imidazol-1-yl]bicyclo[1.1.1]pentan-1-yl}-4-oxo-3,4-dihydro-2H-1-benzopyran-2-carboxamide (Compound 176)

The methodologies described in Example 30D substituting the product of Example 1B for 3-(2-(4-chloro-3-fluorophenoxy)acetamido)bicyclo[1.1.1]pentane-1-carboxylic acid and substituting Example 49B for Example 30C gave the title compound. ¹H NMR (400 MHz, DMSO-d₆) δ ppm 9.19 (s, 1H), 8.40 (s, 1H), 8.06 (d, J=1.4 Hz, 1H), 7.80 (ddd, J=12.1, 7.8, 2.2 Hz, 1H), 7.70-7.61 (m, 2H), 7.61 (ddd, J=6.0, 4.5, 2.1 Hz, 1H), 7.51 (dt, J=10.7, 8.5 Hz, 1H), 7.24-7.15 (m, 1H), 5.17 (dd, J=8.2, 6.0 Hz, 1H), 3.03-2.96 (m, 2H), 2.57 (s, 6H); MS (APCI⁺) m/z 470 (M+H)⁺.

### Example 78: (2S)-6-chloro-4-oxo-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide (Compound 177)

The methodologies described in Example 30D substituting the product of Example 10A for 3-(2-(4-chloro-3-fluorophenoxy)acetamido)bicyclo[1.1.1]pentane-1-carboxylic acid and substituting Example 53C for Example 30C gave the title compound. ¹H NMR (400 MHz, CDCl₃) δ ppm 7.89 (d, J=2.6 Hz, 1H), 7.50 (dd, J=8.8, 2.6 Hz, 1H), 7.06 (d, J=8.8 Hz, 1H), 6.57 (d, J=7.8 Hz, 1H), 4.92 (dd, J=13.2, 3.4 Hz, 1H), 4.78-4.66 (m, 2H), 4.26-4.09 (m, 2H), 3.47-3.29 (m, 2H), 3.20 (dd, J=17.3, 3.4 Hz, 1H), 2.88 (dddd, J=13.2, 11.1, 5.4, 3.0 Hz, 3H), 2.71 (tdd, J=9.2, 7.0, 4.0 Hz, 2H), 2.33 (dtt, J=13.2, 4.6, 2.3 Hz, 1H), 2.32-2.19 (m, 1H), 2.22-2.10 (m, 1H), 1.82-1.70 (m, 1H); MS (APCI⁺) m/z 516 (M+H)⁺.

### Example 79: (2S,4R)-6-chloro-N-trans-4-[3-(4-chlorophenyl)azetidine-1-carbonyl]cyclohexyl)-4-hydroxy-3,4-dihydro-2H-1-benzopyran-2-carboxamide (Compound 178)

The reaction and purification conditions described in Example 3C substituting the product of Example 74A for the product of Example 3A, and the product of Example 73B for the product of Example 3B gave the title compound. ¹H NMR (400 MHz, DMSO-d₆) δ ppm 7.87 (d, J=8.1 Hz, 1H), 7.39-7.29 (m, 4H), 7.24 (d, J=2.6 Hz, 1H), 7.17 (dd, J=8.8, 2.7 Hz, 1H), 6.86 (d, J=8.8 Hz, 1H), 5.55 (br s, 1H), 4.55-4.45 (m, 3H), 4.16 (t, J=8.9 Hz, 1H), 4.08 (dd, J=8.5, 6.0 Hz, 1H), 3.84-3.72 (m, 1H), 3.71 (dd, J=9.2, 6.1 Hz, 1H), 3.56-3.45 (m, 1H), 2.15-2.04 (m, 1H), 2.05-1.96 (m, 1H), 1.85 (ddd, J=14.1, 10.7, 3.8 Hz, 1H), 1.79-1.64 (m, 4H), 1.39-1.10 (m, 4H); MS (APCI⁺) m/z 503 (M+H)⁺.

### Example 80: (2R)-6-chloro-N-{3-[3-(4-chlorophenyl)-2-oxoimidazolidin-1-yl]bicyclo[1.1.1]pentan-1-yl}-4-oxo-3,4-dihydro-2H-1-benzopyran-2-carboxamide (Compound 179)

### Example 80A: tert-butyl{3-[3-(4-chlorophenyl)-2-oxoimidazolidin-1-yl]bicyclo[1.1.1.]pentan-1-yl}carbamate

The reaction and purification conditions described in Example 1A substituting 1-(4-chlorophenyl)imidazolidin-2-one (Enamine) for metaxalone gave the title compound. MS (APCI⁺) m/z 378 (M+H)⁺.

### Example 80B: (2R)-6-chlor-N-{3-[3-(4-chlorophenyl)-2-oxoimidazolidin-1-yl]bicyclo[1.1.1]pentan-1-yl}-4-oxo-3,4-dihydro-2H-1-benzopyran-2-carboxamide

The reaction and purification conditions described in Example 1C substituting the product of Example 80A for the product of Example 1A gave the title compound. ¹H NMR (400 MHz, DMSO-d₆) δ ppm 9.00 (s, 1H), 7.68-7.60 (m, 2H), 7.60-7.52 (m, 2H), 7.39-7.30 (m, 2H), 7.22-7.14 (m, 1H), 5.10 (dd, J=7.8, 6.4 Hz, 1H), 3.81-3.72 (m, 2H), 3.48-3.41 (m, 2H), 2.99-2.92 (m, 2H), 2.30 (s, 6H); MS (APCI⁺) m/z 486 (M+H)⁺.

### Example 81: (2S,4S)-6-chloro-N-{3-[4-(3,4-difluorophenyl)-1H-imidazol-1-yl]bicyclo[1.1.1]pentan-1-yl}-4-hydroxy-3,4-dihydro-2H-1-benzopyran-2-carboxamide (Compound 180)

The methodologies described in Example 5 substituting Example 76 for Example 4 and purifying by preparative HPLC (Phenomenex® Luna® C8(2) 5 μm AXIA™ column (150 mm×30 mm) using a 30-100% gradient of acetonitrile (A) and 0.1% trifluoroacetic acid in water (B) over 25 minutes, at a flow rate of 50 mL/minute) gave the title compound. ¹H NMR (400 MHz, DMSO-d₆) δ ppm 8.91 (s, 1H), 8.51 (s, 1H), 8.09 (d, J=1.5 Hz, 1H), 7.84-7.72 (m, 1H), 7.64-7.56 (m, 1H), 7.50 (dt, J=10.7, 8.5 Hz, 1H), 7.36 (dd, J=2.7, 0.9 Hz, 1H), 7.18 (dd, J=8.7, 2.7 Hz, 1H), 6.90-6.77 (m, 1H), 4.80 (dd, J=10.6, 5.9 Hz, 1H), 4.64 (dd, J=11.9, 2.3 Hz, 1H), 2.57 (s, 6H), 2.44-2.26 (m, 2H), 1.77-1.60 (m, 1H), MS (APCI⁺) m/z 472 (M+H)⁺.

### Example 82: (2R,4R)-6-chloro-N-{3-[4-(3,4-difluorophenyl)-1H-imidazol-1-yl]bicyclo[1.1.1]pentan-1-yl}-4-hydroxy-3,4-dihydro-2H-1-benzopyran-2-carboxamide (Compound 181)

The methodologies described in Example 5 substituting Example 77 for Example 4 and purifying by preparative HPLC (Phenomenex® Luna® C8(2) 5 μm AXIA™ column (150 mm×30 mm) using a 30-100% gradient of acetonitrile (A) and 0.1% trifluoroacetic acid in water (B) over 25 minutes, at a flow rate of 50 mL/minute) gave the title compound. ¹H NMR (500 MHz, DMSO-d₆) δ ppm 8.95 (s, 1H), 8.42 (s, 1H), 8.08 (s, 1H), 7.81 (ddd, J=12.0, 7.8, 2.1 Hz, 1H), 7.62 (ddd, J=8.0, 3.8, 2.0 Hz, 1H), 7.52 (dt, J=10.7, 8.6 Hz, 1H), 7.40 (d, J=2.7 Hz, 1H), 7.22 (dd, J=8.7, 2.7 Hz, 1H), 6.90 (d, J=8.7 Hz, 1H), 4.83 (dd, J=10.7, 5.8 Hz, 1H), 4.67 (dd, J=11.9, 2.3 Hz, 1H), 2.59 (s, 6H), 2.43-2.35 (m, 1H), 1.73 (td, J=12.6, 10.8 Hz, 1H); MS (APCI⁺) m/z 472 (M+H)⁺.

### Example 83: (2R,4R)-6-chloro-4-hydroxy-N-[trans-4-(3-phenylazetidine-1-carbonyl)cyclohexyl]-3,4-dihydro-2H-1-benzopyran-2-carboxamide (Compound 182)

### Example 83A: Tert-Butyl ((trans)-4-(3-phenylazetidine-1-carbonyl)cyclohexyl)carbamate

Palladium hydroxide on carbon (moistened, 10-20% dry basis, 1.5 mg) was added to a solution of the product of Example 74A (15.4 mg, 0.039 mmol) in methanol (2 mL) in a 4 mL-vial followed by addition of sodium borohydride (5.9 mg, 0.157 mmol). After stirring at ambient temperature for 10 minutes, more sodium borohydride (5.9 mg, 0.157 mmol) was added. The vial was flushed with nitrogen, sealed and stirred for another 2 hours. Water (0.2 mL) was added. The resulting mixture was stirred for 10 minutes, filtered through a syringe filter and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (13 mg, 0.036 mmol, 93% yield). MS (APCI⁺) m/z 359 (M+H)⁺.

### Example 83B: (2R)-6-chloro-4-oxo-N-[trans-4-(3-phenylazetidine-1-carbonyl)cyclohexyl]-3,4-dihydro-2H-1-benzopyran-2-carboxamide

The reaction and purification conditions described in Example 1C substituting the product of Example 83A for the product of Example 1A gave the title compound. MS (APCI⁺) m/z 467 (M+H)⁺.

### Example 83C: (2R,4R)-6-chloro-4-hydroxy-N-[trans-4-(3-phenylazetidine-1-carbonyl)cyclohexyl]-3,4-dihydro-2H-1-benzopyran-2-carboxamide

The reaction and purification conditions described in Example 6C substituting the product of Example 83B for the product of Example 6B gave the title compound. ¹H NMR (400 MHz, DMSO-d₆) δ ppm 7.88 (d, J=8.1 Hz, 1H), 7.41-7.32 (m, 5H), 7.32-7.22 (m, 1H), 7.19 (dd, J=8.7, 2.7 Hz, 1H), 6.88 (d, J=8.7 Hz, 1H), 5.72 (br s, 1H), 4.80 (dd, J=10.7, 6.0 Hz, 1H), 4.64-4.53 (m, 2H), 4.29-4.19 (m, 1H), 4.19-4.12 (m, 1H), 3.90-3.76 (m, 2H), 3.64-3.53 (m, 1H), 2.34 (ddd, J=13.0, 6.0, 2.3 Hz, 1H), 2.24-2.13 (m, 1H), 1.85-1.64 (m, 5H), 1.47-1.26 (m, 4H); MS (APCI⁺) m/z 469 (M+H)⁺.

### Example 84: (2R,4R)-6-chloro-N-{3-[3-(4-chlorophenyl)-2-oxoimidazolidin-1-yl]bicyclo[1.1.1]pentan-1-yl}-4-hydroxy-3,4-dihydro-2H-1-benzopyran-2-carboxamide (Compound 183)

The reaction and purification conditions described in Example 6C substituting the product of Example 80 for the product of Example 6B gave the title compound. ¹H NMR (400 MHz, DMSO-d₆) δ ppm 8.75 (s, 1H), 7.61-7.52 (m, 2H), 7.40-7.28 (m, 3H), 7.20 (dd, J=8.7, 2.7 Hz, 1H), 6.89 (d, J=8.7 Hz, 1H), 5.66 (br s, 1H), 4.81 (dd, J=10.6, 5.9 Hz, 1H), 4.60 (dd, J=12.0, 2.3 Hz, 1H), 3.77 (dd, J=9.3, 6.6 Hz, 2H), 3.48-3.42 (m, 2H), 2.41-2.33 (m, 1H), 2.32 (s, 6H), 1.70 (td, J=12.3, 10.7 Hz, 1H); MS (APCI⁺) m/z 488 (M+H)⁺.

### Example 85: (2R)-6-chloro-4-oxo-N-[(3R,6S)-6-{5-[cis-3-(trifluoromethoxy)cyclobutyl]-1,3,4-oxadiazol-2-yl}oxan-3-yl]-3,4-dihydro-2H-1-benzopyran-2-carboxamide (Compound 184)

The methodologies described in Example 30D substituting the product of Example 1B for 3-(2-(4-chloro-3-fluorophenoxy)acetamido)bicyclo[1.1.1]pentane-1-carboxylic acid and substituting Example 53C for Example 30C gave the title compound. ¹H NMR (400 MHz, CDCl₃) δ ppm 7.89 (d, J=2.6 Hz, 1H), 7.50 (ddd, J=8.8, 2.7, 0.6 Hz, 1H), 7.07 (d, J=8.8 Hz, 1H), 6.61 (d, J=7.9 Hz, 1H), 4.92 (dd, J=12.9, 3.4 Hz, 1H), 4.78-4.66 (m, 2H), 4.17 (dddd, J=14.2, 12.6, 6.5, 2.9 Hz, 2H), 3.50-3.38 (m, 1H), 3.41-3.29 (m, 1H), 3.24-3.14 (m, 1H), 2.96-2.82 (m, 3H), 2.76-2.65 (m, 3H), 2.26 (s, 1H), 2.16 (qd, J=10.0, 9.5, 4.6 Hz, 2H), 1.75-1.64 (m, 1H); MS (APCI⁺) m/z 516 (M+H)⁺.

### Example 86: (2S,4R)-6-chloro-4-hydroxy-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide (Compound 185)

### Example 86A: Tert-Butyl (rac-(1R,2S,4R)-5-((benzyloxy)methyl)-7-oxabicyclo[2.2.1]heptan-2-yl)carbamate

The title compound was synthesized using the same procedure as described in Example 69E substituting 2-(trimethylsilyl)ethanol with tert-butanol. ¹H NMR (400 MHz, CDCl₃) δ ppm 7.29-7.39 (m, 5H), 4.71 (br d, J=6.50 Hz, 1H), 4.42-4.61 (m, 3H), 4.24-4.36 (m, 1H), 3.60-3.72 (m, 1H), 3.60-3.72 (m, 1H), 3.47-3.58 (m, 1H), 3.15-3.33 (m, 1H), 2.40 (tq, J=10.43, 5.14 Hz, 1H), 2.23 (br dd, J=13.45, 8.07 Hz, 1H), 1.89 (td, J=11.94, 6.00 Hz, 1H), 1.35-1.53 (m, 9H), 1.29-1.33 (m, 1H), 0.81-0.98 (m, 1H).

### Example 86B: Tert-Butyl (rac-(1R,2S,4R)-5-(hydroxymethyl)-7-oxabicyclo[2.2.1]heptan-2-yl)carbamate

The title compound was synthesized using the same procedure as described in Example 69F substituting the product of Example 69E with the product of Example 86A. ¹H NMR (400 MHz, CDCl₃) δ ppm 4.77 (br d, J=7.38 Hz, 1H), 4.58 (t, J=5.07 Hz, 1H), 4.32 (br d, J=5.88 Hz, 1H), 3.65-3.84 (m, 3H), 3.48 (t, J=10.01 Hz, 1H), 2.19-2.40 (m, 2H), 1.83-1.95 (m, 3H), 1.44 (s, 9H), 1.34 (dt, J=13.45, 4.35 Hz, 1H), 0.87-1.00 (m, 1H).

### Example 86C: Tert-Butyl (rac-(1R,2S,4R)-5-cyano-7-oxabicyclo[2.2.1]heptan-2-yl)carbamate

The title compound was synthesized using the same procedure as described in Example 69G substituting the product of Example 69F with the product of Example 86B. ¹H NMR (400 MHz, CDCl₃) δ ppm 4.75 (t, J=5.07 Hz, 1H), 4.65 (br s, 1H), 4.51 (br d, J=5.63 Hz, 1H), 3.94 (br s, 1H), 2.77-2.87 (m, 1H), 2.62 (dd, J=14.01, 8.13 Hz, 1H), 2.22 (td, J=12.35, 5.82 Hz, 1H), 1.74 (br dd, J=12.82, 5.32 Hz, 1H), 0.83-0.92 (m, 2H) 0.94-1.01 (m, 1H) 1.23-1.33 (m, 1H) 1.41-1.48 (m, 9H) 1.49-1.52 (m, 1H).

### Example 86D: rac-(1R,2S,4R,5S)-5-((tert-butoxycarbonyl)amino)-7-oxabicyclo[2.2.1]heptane-2-carboxylic Acid

The title compound was synthesized using the same procedure as described in Example 69H substituting the product of Example 69G with the product of Example 86C. ¹H NMR (400 MHz, DMSO-d₆) δ ppm 12.14 (br s, 2H), 7.21-7.43 (m, 6H), 4.60 (d, J=5.63 Hz, 1H), 4.39-4.52 (m, 4H), 3.47-3.56 (m, 1H), 2.51-2.57 (m, 2H), 2.16-2.34 (m, 2H), 1.70-1.89 (m, 3H), 1.02 (dd, J=11.94, 5.19 Hz, 1H).

### Example 86E: Tert-Butyl (rac-(1R,2S,4R,5S)-5-(2-(cis-3-(trifluoromethoxy)cyclobutanecarbonyl)hydrazinecarbonyl)-7-oxabicyclo[2.2.1]heptan-2-yl)carbamate

The title compound was synthesized using the same procedure as described in Example 64E substituting the product of Example 64C with the product of Example 86D. ¹H NMR (400 MHz, DMSO-d₆) δ ppm 9.77 (d, J=1.8 Hz, 1H), 9.65 (d, J=1.7 Hz, 1H), 6.76 (d, J=6.4 Hz, 1H), 4.74 (p, J=7.5 Hz, 1H), 4.58-4.41 (m, 1H), 4.20 (d, J=5.5 Hz, 1H), 2.65 (tt, J=9.3, 7.8 Hz, 1H), 2.43 (dd, J=9.0, 4.5 Hz, 1H), 2.24 (dd, J=11.1, 8.3 Hz, 2H), 1.94-1.77 (m, 2H), 1.58-1.40 (m, 2H), 1.34 (s, 9H).

### Example 86F: Tert-Butyl (rac-(1R,2S,4R,5S)-5-(5-(cis-3-(trifluoromethoxy)cyclobutyl)-1,3,4-oxadiazol-2-yl)-7-oxabicyclo[2.2.1]heptan-2-yl)carbamate

The title compound was synthesized using the same procedures described in Example 25Q substituting the product of Example 25P with the product of Example 86E. ¹H NMR (400 MHz, DMSO-d₆) δ ppm 6.89 (d, J=6.2 Hz, 1H), 4.89 (p, =7.5 Hz, 1H), 4.71 (d, J=5.5 Hz, 1H), 4.38 (d, J=5.5 Hz, 1H), 3.59 (t, J=4.4 Hz, 1H), 3.46-3.35 (m, 1H), 3.24 (dd, 0.1=8.9, 4.5 Hz, 1H), 2.83 (tdt, J=9.7, 7.4, 2.4 Hz, 2H), 2.51-2.35 (m, 1H), 2.09-1.96 (m, 2H), 1.91 (dd, J=12.6, 8.9 Hz, 1H), 1.63-1.52 (m, 1H), 1.39 (s, 9H); MS (DCI⁺) m/z 420.3 (M+H)⁺.

### Example 86G: (rac-(1R,2S,4R,5S)-5-(5-(cis-3-(trifluoromethoxy)cyclobutyl)-1,3,4-oxadiazol-2-yl)-7-oxabicyclo[2.2.1]heptan-2-amine Trifluoroacetic Acid

To a solution of the product of Example 86F (0.22 g, 0.525 mmol) in dichloromethane (5.0 mL) was added 2,2,2-trifluoroacetic acid (2.5 mL, 32.4 mmol). The reaction mixture was stirred at ambient temperature for one hour. Solvent and excess 2,2,2-trifluoroacetic acid were removed under high vacuum to give 0.24 g of the title compound, which was used without further purification. MS (DCI⁺) m/z 320.2 (M+H)⁺.

### Example 86H: (2S,4R)-6-chloro-4-hydroxy-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide

To a solution of the product of Example 86G (27 mg, 0.044 mmol), the product of Example 73B (12.47 mg, 0.055 mmol) and N-ethyl-N-isopropylpropan-2-amine (0.030 mL, 0.174 mmol) in N,N-dimethylformamide (1 mL) was added 2-(3H-[1,2,3]triazolo[4,5-b]pyridin-3-yl)-1,1,3,3-tetramethylisouronium hexafluorophosphate(V) (24.88 mg, 0.065 mmol) and the mixture was stirred at ambient temperature for 30 minutes. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 13 mg of the title compound. ¹H NMR (400 MHz, DMSO-d₆) δ ppm 8.10 (dd, J=6.7, 3.9 Hz, 1H), 7.31 (d, J=2.7 Hz, 1H), 7.24 (dt, J=8.8, 2.1 Hz, 1H), 6.93 (d, J=8.7 Hz, 1H), 4.90 (p, J=7.4 Hz, 1H), 4.80 (d, J=5.5 Hz, 1H), 4.62 (ddd, J=14.2, 7.1, 3.5 Hz, 2H), 4.47 (dd, J=10.0, 5.4 Hz, 1H), 3.95 (dq, J=11.3, 4.1 Hz, 1H), 3.31 (dd, J=8.8, 4.7 Hz, 1H), 2.83 (dtd, J=10.2, 7.4, 3.0 Hz, 2H), 2.19-1.89 (m, 5H), 1.72 (ddq, J=12.9, 8.8, 5.5, 4.4 Hz, 1H).

### Example 87: (2S,4S)-6-chloro-4-hydroxy-N-[(1RS,2SR,4RS,5SR)-5-5-[cis-3-(trifluoromethoxy)cyclobutyl]-1,3,4-oxadiazol-2-yl)-7-oxabicyclo[2.2.1]heptan-2-yl]-3,4-dihydro-2H-1-benzopyran-2-carboxamide (Compound 186)

### Example 87A: (2S)-6-chloro-4-oxo-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide

To the mixture of the product of Example 86G (75.0 mg, 0.121 mmol), the product of Example 10A (34.3 mg, 0.151 mmol) and N-ethyl-N-isopropylpropan-2-amine (0.085 mL, 0.485 mmol) in N,N-dimethylformamide (1 mL) was added 2-(3H-[1,2,3]triazolo[4,5-b]pyridin-3-yl)-1,1,3,3-tetramethylisouronium hexafluorophosphate(V) (69.1 mg, 0.182 mmol) and the mixture was stirred at ambient temperature for 30 minutes. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 44 mg of the title compound. ¹H NMR (400 MHz, DMSO-d₆) δ ppm 8.37 (d, J=6.8 Hz, 1H), 7.67-7.58 (m, 2H), 7.16 (dd, J=8.6, 1.1 Hz, 1H), 5.13 (ddd, J=8.2, 5.7, 3.5 Hz, 1H), 4.90 (p, J=7.6 Hz, 1H), 4.81 (d, J=5.5 Hz, 1H), 4.45 (d, J=5.5 Hz, 0H), 4.36 (d, J=5.5 Hz, 1H), 3.90 (td, J=7.6, 3.4 Hz, 1H),3.32-3.25 (m, 1H), 3.05-2.91 (m, 2H), 2.93-2.77 (m, 2H), 2.22-2.01 (m, 2H), 1.98 (ddd, J=12.3, 8.9, 2.9 Hz, 1H), 1.63 (ddt, J=17.8, 13.1, 4.5 Hz, 1H).

### Example 87B: (2S,4S)-6-chloro-4-hydroxy-N-[(1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide

The title compound was synthesized using the same procedure as described in Example 6C substituting the product of Example 6B with the product of Example 87A. ¹H NMR (400 MHz, DMSO-d₆) δ ppm 8.02 (t, J=7.5 Hz, 1H), 7.38 (d, J=2.7 Hz, 1H), 7.19 (dt, J=8.7, 2.4 Hz, 1H), 6.88 (d, J=8.7 Hz, 1H), 5.63 (s, 1H), 4.90 (p, J=7.5 Hz, 1H), 4.80 (q, J=6.6, 5.7 Hz, 2H), 4.66 (dt, J=11.7, 2.5 Hz, 1H), 4.48 (t, J=5.8 Hz, 1H), 3.95 (ddq, J=8.2, 5.6, 2.8 Hz, 1H), 3.30 (d, J=4.7 Hz, 1H), 2.89-2.78 (m, 2H), 2.54 (t, J=3.7 Hz, 0H), 2.38-2.27 (m, 1H), 2.19-1.96 (m, 3H), 1.84-1.73 (m, 1H), 1.71 (dq, J=13.1, 5.1, 4.5 Hz, 1H); MS (APCI⁺) m/z 530.64 (M+H)⁺.

### Example 88: (2R,4R)-6-chloro-4-hydroxy-N-[1RS,2SR,4RS,5SR)-5-{5-[cis-3-(trifluoromethoxy)cyclobutyl]-1,3,4-oxadiazol-2-yl}-7-oxabicyclo[2.2.1]heptan-2-yl]-3,4-dihydro-2H-1-benzopyran-2-carboxamide (Compound 187)

The title compound was synthesized using the same procedures described in Example 87A through Example 87B substituting the product of Example 10A with the product of Example 1B. ¹H NMR (400 MHz, DMSO-d₆) δ ppm 8.02 (dd, J=8.2, 6.8 Hz, 1H), 7.38 (d, J=2.7 Hz, 1H), 7.19 (dt, J=8.7, 2.4 Hz, 1H), 6.88 (d, J=8.7 Hz, 1H), 5.69 (s, 1H), 4.90 (p, J=7.5 Hz, 1H), 4.80 (q, J=6.8, 5.7 Hz, 2H), 4.66 (dt, J=11.8, 2.5 Hz, 1H), 4.48 (t, J=5.7 Hz, 1H), 3.95 (ddt, J=8.2, 5.8, 2.7 Hz, 1H), 3.47-3.34 (m, 11H), 3.31 (dd, J=8.8, 4.7 Hz, 1H), 2.83 (dtd, J=10.1, 7.4, 2.9 Hz, 2H), 2.38-2.27 (m, 1H), 2.19-1.93 (m, 3H), 1.84-1.73 (m, 1H), 1.76-1.66 (m, 1H); MS (APCI⁺) m/z 530.64 (M+H)⁺.

### Example 89: (2R)-6-chloro-4-oxo-N-[(1r,4R)-4-{2-oxo-3-[3-(trifluoromethoxy)cyclobutyl]imidazolidin-1-yl}cyclohexyl]-3,4-dihydro-2H-1-benzopyran-2-carboxamide (Compound 188)

### Example 89A: Benzyl ((1r,4r)-4-(2-oxo-3-(3-(trifluoromethoxy)cyclobutyl)imidazolidin-1-yl)cyclohexyl)carbamate

The reaction and purification conditions described in Example 1A substituting the product of Example 250 for 3-((tert-butoxycarbonyl)amino)bicyclo[1.1.1]pentane-1-carboxylic acid, and the product of Example 37C for metaxalone gave the title compound. MS (APCI⁺) m/z 456 (M+H)⁺.

### Example 89B: (2R)-6-chloro-4-oxo-N-[(1r,4R)-4-{2-oxo-3-[3-(trifluoromethoxy)cyclobutyl]imidazolidin-1-yl}cyclohexyl]-3,4-dihydro-2H-1-benzopyran-2-carboxamide

The reaction and purification conditions described in Example 1C substituting the product of Example 89A for the product of Example 1A, and also raising the reaction temperature for the first step from ambient temperature in trifluoroacetic acid to 70° C. in trifluoroacetic acid gave the title compound. ¹H NMR (600 MHz, DMSO-d₆) δ ppm 8.16 (dd, J=8.0, 1.4 Hz, 1H), 7.67-7.61 (m, 2H), 7.17 (dd, J=8.7, 0.6 Hz, 1H), 5.11 (dd, J=7.9, 5.7 Hz, 1H), 4.89 (tt, J=7.1, 3.6 Hz, 0.4H, trans cyclobutane), 4.59 (p, J=7.2 Hz, 0.6H, cis cyclobutane), 4.47-4.39 (m, 0.4H, trans cyclobutane), 4.08-3.88 (m, 0.6H, cis cyclobutane), 3.56-3.42 (m, 2H), 3.31 (d, J=1.9 Hz, 2H), 3.25-3.19 (m, 2H), 3.03-2.91 (m, 2H), 2.50-2.43 (m, 2H), 2.38-2.32 (m, 2H), 1.84-1.78 (m, 1H), 1.74-1.69 (m, 1H), 1.60-1.43 (m, 4H), 1.39-1.25 (m, 2H); MS (APCI⁺) m/z 530 (M+H)⁺.

### Example 90: (2R)-6,7-difluoro-4-oxo-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 189)

### Example 90A: Tert-Butyl [4-(2-{[cis-3-(trifluoromethoxy)cyclobutyl]oxy}acetamido)bicyclo[2.2.2]octan-1-yl]carbamate

The reaction and purification conditions described in Example 2B substituting tert-butyl (4-aminobicyclo[2.2.2]octan-1-yl)carbamate for the product of Example 2A, and the product of Example 13P for the product of Example 1B gave the title compound. MS (APCI⁺) m/z 437 (M+H)⁺.

### Example 90B: (E)-4-(4,5-difluoro-2-hydroxyphenyl)-4-oxobut-2-enoic Acid

Maleic anhydride (1.90 g, 19.37 mmol) and aluminum chloride (5.17 g, 38.7 mmol) were combined with dichloroethane (20 mL) and stirred at 50° C. for 2 minutes. 3,4-Difluoroanisole (2.0 mL, 16.85 mmol) was added dropwise over a period of 2 minutes. The resulting reaction mixture was stirred at 50° C. for 5 hours and then at ambient temperature for 18 hours before being poured into a mixture of concentrated aqueous HCl (11.6 M, 20 mL) and ice (about 100 grams). After all ice was melted and while the mixture was still cold, the precipitate was collected by filtration through filter paper, and dried in a 40° C. vacuum oven overnight to give the title compound (1.54 g, 6.75 mmol, 40% yield). ¹H NMR (DMSO-d₆) δ ppm 13.00 (br s, 1H), 11.67 (s, 1H), 7.90 (d, J=15.5 Hz, 1H), 7.83 (dd, J=11.4, 9.4 Hz, 1H), 7.11-7.05 (m, 1H), 6.65 (d, J=15.4 Hz, 1H), MS (ESI⁻) m/z 227 (M−H)⁻.

### Example 90C: 6,7-difluoro-4-oxochroman-2-carboxylic Acid

The product of Example 90B (340 mg, 1.49 mmol) was suspended in water (7.45 mL) and stirred at ambient temperature. Aqueous NaOH (1.0 M, 1.64 mL) was added dropwise over a period of 2 minutes. The reaction mixture was heated to 100° C. and stirred at that temperature for 2 minutes and then cooled to ambient temperature over a period of 15 minutes. Aqueous HCl (6.0 M) was added dropwise to adjust the pH to about 1. The resulting milky solution was partitioned between dichloromethane (2×30 mL) and water (10 mL). The organic layers were combined, dried over sodium sulfate and concentrated under reduced pressure. The resulting residue was purified by preparative HPLC [YMC TriArt™ C18 Hybrid 5 μm column. 50×100 mm, flow rate 140 mL/minute, 0-100% gradient of acetonitrile in buffer (0.1% trifluoroacetic acid)] to give the title compound (0.2 g, 0.88 mmol, 59% yield). MS (ESI⁻) m/z 227 (M−H)⁻.

### Example 90D: (R)-6,7-difluoro-4-oxochroman-2-carboxylic Acid

The product of Example 90C was purified by preparative chiral HPLC [CHIRALPAK® AD-H 5 μm column, 20×250 mm, flow rate 6 mL/minute, 80% ethanol and 0.1% trifluoroacetic acid in heptane (isocratic gradient)] to give the title compound as the earlier eluting fraction. MS (ESI⁻) m/z 227 (M−H)⁻.

### Example 90E: (2R)-6,7-difluoro-4-oxo-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide

The reaction and purification conditions described in Example 1C substituting the product of Example 90A for the product of Example 1A, and the product of Example 90D for the product of Example 1B gave the title compound. ¹H NMR (500 MHz, DMSO-d₆) δ ppm 7.69 (s, 1H), 7.66 (dd, J=10.3, 9.2 Hz, 1H), 7.30 (dd, J=11.5, 6.5 Hz, 1H), 6.99 (s, 1H), 5.06 (dd, J=8.4, 5.0 Hz, 1H), 4.47 (p, J=7.1 Hz, 1H), 3.73-3.63 (m, 1H), 3.67 (s, 2H), 2.98-2.84 (m, 2H), 2.77-2.68 (m, 2H), 2.16-2.07 (m, 2H), 1.90-1.83 (m, 12H); MS (APCI⁺) m/z 547 (M+H)⁺.

### Example 91: (2S,4S)-6-chloro-4-hydroxy-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide (Compound 190)

The title compound was synthesized using the same procedures as described in Example 87A through Example 87B substituting the product of Example 86G with product of Example 64G. ¹H NMR (400 MHz, DMSO-d₆) δ ppm 7.67 (s, 1H), 7.38 (d, J=2.6 Hz, 1H), 7.19 (dd, J=8.7, 2.7 Hz, 1H), 6.87 (d, J=8.7 Hz, 1H), 5.68 (s, 1H), 4.90 (p, J=7.5 Hz, 1H), 4.79 (dd, J=10.6, 5.9 Hz, 1H), 4.60 (dd, J=11.7, 2.3 Hz, 1H), 4.05 (t, J=1.9 Hz, 2H), 3.49-3.40 (m, 1H), 2.85 (tdt, J=9.7, 7.4, 2.3 Hz, 2H), 2.51-2.44 (m, 1H), 2.41-2.13 (m, 6H), 2.13-1.98 (m, 4H), 1.77 (dt, J=12.8, 10.9 Hz, 1H); MS (APCI⁺) m/z 519.06 (M+H)⁺.

### Example 92: (2R,4R)-6-chloro-4-hydroxy-N-(1-{5-[cis-3-(trifluoromethoxy)cyclobutyl]-1,3,4-oxadiazol-2-yl}-2-oxabicyclo[2.2.2]octan-4-yl)-3,4-dihydro-2H-1-benzopyran-2-carboxamide (Compound 191)

The title compound was synthesized using the same procedures as described in Example 87A through Example 87B substituting the product of Example 86G with the product of Example 64G and the product of Example 10A with the product of Example 1B. ¹H NMR (400 MHz, DMSO-d₆) δ ppm 7.66 (s, 1H), 7.38 (d, J=2.5 Hz, 1H), 7.19 (dd, J=8.8, 2.7 Hz, 1H), 6.87 (d, J=8.7 Hz, 1H), 4.90 (p, J=7.4 Hz, 1H), 4.79 (dd, J=10.5, 5.9 Hz, 1H), 4.60 (dd, J=11.7, 2.3 Hz, 1H), 4.10-4.00 (m, 2H), 2.85 (dtd, J=9.9, 7.4, 2.8 Hz, 2H), 2.50-2.43 (m, 1H), 2.41-2.27 (m, 3H), 2.31-2.20 (m, 1H), 2.23-2.16 (m, 1H), 2.16 (d, J=8.0 Hz, 1H), 2.13-1.99 (m, 2H), 2.06 (s, 2H), 1.77 (dt, J=12.7, 10.9 Hz, 1H); MS (APCI⁺) m/z 519.06 (M+H)⁺.

### Example 93: (2R)-6-chloro-4-oxo-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 192)

The reaction and purification conditions described in Example 1C substituting the product of Example 90A for the product of Example 1A gave the title compound. ¹H NMR (600 MHz, DMSO-d₆) δ ppm 17.68 (s, 1H), 7.65-7.58 (m, 2H), 7.15 (dd, J=8.8, 0.5 Hz, 1H), 6.98 (s, 1H), 5.04 (dd, J=8.5, 4.8 Hz, 1H), 4.47 (p, J=7.2 Hz, 1H), 3.72-3.64 (m, 1H), 3.67 (s, 2H), 2.98-2.85 (m, 2H), 2.76-2.68 (m, 2H), 2.15-2.06 (m, 2H), 1.92-1.82 (m, 12H); MS (APCI⁺) m/z 545 (M+H)⁺.

### Example 94: (2S,4R)-6-chloro-4-hydroxy-N-[4-({[5-(trifluoromethyl)pyridin-2-yl]methyl}carbamoyl)bicyclo[2.2.2]octan-1-yl]-3,4-dihydro-2H-1-benzopyran-2-carboxamide (Compound 193)

The reaction and purification conditions described in Example 3C substituting the product of Example 58A for the product of Example 3A, and the product of Example 73B for the product of Example 3B gave the title compound. ¹H NMR (500 MHz, DMSO-d₆) δ ppm 8.89-8.85 (m, 1H), 8.20 (t, J=6.0 Hz, 1H), 8.16 (dd, J=8.4, 2.4 Hz, 1H), 7.43 (s, 1H), 7.38 (d, J=8.2 Hz, 1H), 7.31 (d, J=2.6 Hz, 1H), 7.23 (dd, J=8.8, 2.7 Hz, 1H), 6.91 (d, J=8.8 Hz, 1H), 5.61 (s, 1H), 4.61-4.53 (m, 2H), 4.40 (d, J=5.8 Hz, 2H), 2.09-1.99 (m, 1H), 2.02-1.92 (m, 1H), 1.95-1.77 (m, 12H); MS (APCI⁺) m/z 538 (M+H)⁺.

### Example 95: (2R,4R)-6,7-difluoro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 194)

The reaction and purification conditions described in Example 6C substituting the product of Example 90 for the product of Example 6B gave the title compound. ¹H NMR (500 MHz, DMSO-d₆) δ ppm 7.32 (s, 1H), 7.36-7.28 (m, 1H), 7.00 (s, 1H), 6.96 (dd, J=11.9, 7.0 Hz, 1H), 5.70 (s, 1H), 4.74 (dd, J=10.7, 6.0 Hz, 1H), 4.56 (dd, J=11.8, 2.3 Hz, 1H), 4.47 (p, J=7.1 Hz, 1H), 3.73-3.64 (m, 3H), 2.78-2.68 (m, 2H), 2.26 (ddd, J=12.9, 5.9, 2.3 Hz, 1H), 2.17-2.07 (m, 2H), 1.94-1.87 (m, 12H), 1.72 (ddd, J=13.0, 11.9, 10.7 Hz, 1H), MS (APCI⁺) m/z 549 (M+H)⁺.

### Example 96: (2R,4R)-6-chloro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 195)

The reaction and purification conditions described in Example 6C substituting the product of Example 93 for the product of Example 6B gave the title compound. ¹H NMR (500 MHz, DMSO-d₆) δ ppm 7.37 (dd, J=2.7, 1.0 Hz, 1H), 7.31 (s, 1H), 7.18 (dd, J=8.6, 2.7 Hz, 1H), 7.00 (s, 1H), 6.86 (d, J=8.7 Hz, 1H), 5.69 (s, 1H), 4.77 (dd, J=10.7, 5.9 Hz, 1H), 4.55 (dd, J=11.8, 2.2 Hz, 1H), 4.47 (p, J=7.1 Hz, 1H), 3.73-3.64 (m, 1H), 3.68 (s, 2H), 2.78-2.68 (m, 2H), 2.26 (ddd, J=12.9, 6.0, 2.3 Hz, 1H), 2.16-2.06 (m, 2H), 1.96-1.87 (m, 12H), 1.72 (ddd, J=13.0, 11.9, 10.8 Hz, 1H); MS (APCI⁺) m/z 547 (M+H)⁺.

### Example 97: (2R,4R)-6-chloro-4-hydroxy-N-[4-(2-{[cis-3-(trifluoromethoxy)cyclobutyl]oxy}acetamido)bicyclo[2.1.1]hexan-1-yl]-3,4-dihydro-2H-1-benzopyran-2-carboxamide (Compound 196)

### Example 97A: (R)-tert-butyl (4-(6-chloro-4-oxochroman-2-carboxamido)bicyclo[2.1.1]hexan-1-yl)carbamate

The reaction and purification conditions described in Example 2B substituting tert-butyl (4-aminobicyclo[2.1.1]hexan-1-yl)carbamate (Matrix) for the product of Example 2A gave the title compound. MS (APCI⁺) m/z 365 (M−C(CH₃)₃+H)⁺.

### Example 97B: (2R)-6-chloro-4-oxo-N-[4-(2-{[cis-3-(trifluoromethoxy)cyclobutyl]oxy}acetamido)bicyclo[2.1.1]hexan-1-yl]-3,4-dihydro-2H-1-benzopyran-2-carboxamide

The reaction and purification conditions described in Example 1C substituting the product of Example 97A for the product of Example 1A, and the product of Example 13P for the product of Example 1B gave the title compound. MS (APCI⁺) m/z 517 (M+H)⁺.

### Example 97C: (2R,4R)-6-chloro-4-hydroxy-N-[4-(2-{[cis-3-(trifluoromethoxy)cyclobutyl]oxy}acetamido)bicyclo[2.1.1]hexan-1-yl]-3,4-dihydro-2H-1-benzopyran-2-carboxamide

The reaction and purification conditions described in Example 6C substituting the product of Example 97B for the product of Example 6B gave the title compound. ¹H NMR (500 MHz, DMSO-d₆) δ ppm 8.41 (s, 1H), 8.11 (s, 1H), 7.38 (dd, J=2.7, 1.0 Hz, 1H), 7.19 (ddd, J=8.6, 2.7, 0.7 Hz, 1H), 6.89 (d, J=8.7 Hz, 1H), 5.69 (s, 1H), 4.83-4.77 (m, 1H), 4.59 (dd, J=12.0, 2.2 Hz, 1H), 4.49 (p, J=7.2 Hz, 1H), 3.73 (s, 2H), 3.73-3.68 (m, 1H), 2.78-2.70 (m, 2H), 2.34 (ddd, J=12.9, 5.9, 2.3 Hz, 1H), 2.20-2.11 (m, 2H), 2.08-2.04 (m, 2H), 1.85-1.77 (m, 6H), 1.72 (ddd, J=12.8, 12.0, 10.8 Hz, 1H); MS (APCI⁺) m/z 501 (M−H₂O+H)⁺.

### Example 98: (2R,4R)-6-chloro-4-hydroxy-N-[(1r,4R)-4-2-oxo-3-[3-(trifluoromethoxy)cyclobutyl]imidazolidin-1-yl)cyclohexyl]-3,4-dihydro-2H-1-benzopyran-2-carboxamide (Compound 197)

The reaction and purification conditions described in Example 6C substituting the product of Example 89 for the product of Example 6B gave the title compound. ¹H NMR (600 MHz, DMSO-d₆) δ ppm 7.93-7.88 (m, 2H), 7.38 (d, J=2.7 Hz, 1H), 7.20 (dd, J=8.7, 2.7 Hz, 1H), 6.89 (d, J=8.7 Hz, 1H), 5.71 (s, 1H), 4.90 (tt, J=7.1, 3.7 Hz, 0.4H, trans cyclobutane), 4.81 (dd, J=10.7, 6.0 Hz, 1H), 4.64-4.55 (m, 1.6H), 4.03-3.94 (m, 0.6H, cis cyclobutane), 3.62-3.57 (m, 1H), 3.51-3.30 (m, 3H), 3.27-3.21 (m, 2H), 2.67-2.61 (m, 1H), 2.47 (ddd, J=9.8, 5.9, 2.8 Hz, 2H), 2.41-2.31 (m, 3H), 1.85-1.77 (m, 2H), 1.76-1.67 (m, 1H), 1.61-1.55 (m, 2H), 1.58-1.46 (m, 2H), 1.45-1.34 (m, 2H); MS (APCI⁺) m/z 514 (M−H₂O+H)⁺.

### Example 99: (2R,4S)-6-chloro-4-hydroxy-N-[trans-4-({[5-(trifluoromethyl)pyridin-2-yl]methyl}carbamoyl)cyclohexyl]-3,4-dihydro-2H-1-benzopyran-2-carboxamide (Compound 198)

The product of Example 6C (12 mg, 0.023 mmol) was dissolved in trifluoroacetic acid (0.5 mL, 6.49 mmol) and stirred at ambient temperature for 1 hour. The solution was concentrated under reduced pressure. The resulting residue was taken up in acetonitrile (2 mL) and aqueous ammonium hydroxide (1.7 M, 5 mL) was added. The reaction mixture was stirred at ambient temperature for 2 hours and then concentrated under reduced pressure. The residue was taken up in methanol (2 mL) and filtered through a glass microfiber frit. The residue was purified by reversed-phase chiral HPLC [Phenomenex® Lux® i-Cellulose-5 5 μm column, 21.2×150 mm, flow rate 25 mL/minute, 30-60% acetonitrile in buffer (0.025 M aqueous ammonium bicarbonate, pH 8.2)] to give the title compound (6 mg, 0.012 mmol, 50% yield). ¹H NMR (500 MHz, DMSO-d₆) δ ppm 8.91-8.86 (m, 1H), 8.50 (t, J=6.0 Hz, 1H), 8.18 (dd, J=8.3, 2.4 Hz, 1H), 7.97 (d, J=8.1 Hz, 1H), 7.46 (d, J=8.3 Hz, 1H), 7.32 (d, J=2.7 Hz, 1H), 7.25 (dd, J=8.7, 2.7 Hz, 1H), 6.94 (d, J=8.8 Hz, 1H), 5.63 (s, 1H), 4.62-4.54 (m, 2H), 4.43 (d, J=5.9 Hz, 2H), 3.63-3.57 (m, 1H), 2.19 (tt, J=12.1, 3.3 Hz, 1H), 2.09 (dt, J=13.8, 3.3 Hz, 1H), 1.91 (ddd, J=14.2, 10.9, 3.8 Hz, 1H), 1.87-1.77 (m, 4H), 1.51-1.40 (m, 2H), 1.40-1.26 (m, 2H); MS (APCI⁺) m/z 512 (M+H)⁺.

### Example 100: (2S,4S)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide (Compound 199)

### Example 100A: rac-(2R,4R)-6-chloro-4-hydroxy-3,4-dihydro-2H-1-benzopyran-2-carboxylic Acid

The reaction and purification conditions described in Example 3B substituting 6-chloro-4-oxochroman-2-carboxylic acid (Princeton Bio) for the product of Example 1B gave the title compound. MS (ESI⁻) m/z 227 (M−H)⁻.

### Example 100B: (2S,4S)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide

The title compound was prepared using the procedures described for the synthesis of Example 131D, substituting the product from Example 100A for the product from Example 73B. The crude product was purified by chiral SFC separation [Column: CHIRALPAK IG, 10×250 mm, 5 μm, gradient: 40% methanol in CO₂ (isocratic), flow rate: 15 g/minute; column temperature: 40° C.; automatic back-pressure regulator setting: 1700 psi] to give the title compound as the later eluting fraction. ¹H NMR (500 MHz, DMSO-d₆) δ ppm 8.94 (s, 1H), 7.95 (dd, J=9.5, 2.0 Hz, 1H), 7.87 (dd, J=8.5, 2.0 Hz, 1H), 7.84-7.78 (m, 1H), 7.41-7.37 (m, 1H), 7.21 (dd, J=8.5, 2.5 Hz, 1H), 6.89 (d, J=8.5 Hz, 1H), 5.74-5.70 (m, 1H), 4.86-4.78 (m, 1H), 4.64 (dd, J=12.0, 2.5 Hz, 1H), 2.60 (s, 6H), 2.41-2.34 (m, 1H), 1.77-1.67 (m, 1H); MS (ESI) m/z 488 (M−H)⁻.

### Example 101: (2S,4S)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide (Compound 200)

### Example 101A: 6-chloro-4-oxo-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide

To a solution of the product from Example 132A (240 mg, 0.609 mmol) in dichloromethane (10 mL) was added trifluoroacetic acid (5 mL, 64.9 mmol) and the resulting solution was stirred at room temperature for 16 hours. The volatiles were removed in vacuo. The residue was combined with 6-chloro-4-oxochroman-2-carboxylic acid (134 mg, 0.593 mmol) and N,N-diisopropylethylamine (0.311 mL, 1.780 mmol) in N,N-dimethylformamide (5 mL). (1-[Bis(dimethylamino)methylene]-1H-1,2,3-triazolo[4,5-b]pyridinium 3-oxid hexafluorophosphate)(HATU, 271 mg, 0.712 mmol) was added, and the reaction mixture stirred at room temperature for 16 hours. The mixture was diluted with dichloromethane (50 mL) and washed with brine (3×50 mL), and the combined organic extract were dried over Na₂SO₄, filtered and concentrated in vacuo. The residue was purified by column chromatography on silica gel using a solvent gradient of 0-100% ethyl acetate in isohexane to give the title compound (186 mg, 0.289 mmol, 48.6% yield). ¹H NMR (500 MHz, DMSO-d₆) δ ppm 9.19 (s, 1H), 9.14 (d, J=2.1 Hz, 1H), 8.36 (dd, J=8.4, 2.1 Hz, 1H), 8.12 (d, J=1.3 Hz, 1H), 7.93-7.88 (m, 2H), 7.70-7.64 (m, 2H), 7.20 (dd, J=8.5, 0.8 Hz, 1H), 5.17 (dd, J=8.9, 5.4 Hz, 1H), 3.02-2.97 (m, 2H), 2.56 (s, 6H).

### Example 101B: (2S,4S)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide

The product from Example 10A (163 mg, 0.324 mmol) was suspended in methanol (7 mL) and cooled to 0° C. Sodium borohydride (16 mg, 0.42 mmol) was added slowly portionwise. The reaction mixture was stirred at 0° C. for 1 hour and was quenched with 1 M HCl (25 mL) and extracted with ethyl acetate (40 mL×3). The combined organic extract was dried over MgSO₄, filtered and concentrated in vacuo. The residue was purified by silica gel column chromatography (0-10% methanol in dichloromethane) to afford 6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide (105 mg), which was subjected to chiral SFC separation [Column: Chiralpak® IG, 10×250 mm, 5 μm, gradient: 35% methanol in CO₂ (isocratic), flow rate: 15 g/minute; column temperature: 40° C.; automatic back-pressure regulator setting: 1700 psi] to give the title compound as the later eluting fraction. (15 mg, 9%). ¹H NMR (500 MHz, DMSO-d₆) δ ppm 9.14 (d, J=2.0 Hz, 1H), 8.93 (s, 1H), 8.36 (dd, J=8.0, 2.0 Hz, 1H), 8.13 (d, J=1.5 Hz, 1H), 7.94-7.87 (m, 2H), 7.41-7.38 (m, 1H), 7.24-7.18 (m, 1H), 6.90 (d, J=8.5 Hz, 1H), 5.73 (s, 1H), 4.83 (dd, J=10.5, 6.0 Hz, 1H), 4.67 (dd, J=12.0, 2.5 Hz, 1H), 2.58 (s, 6H), 2.42-2.34 (m, 1H), 1.78-1.69 (m, 1H); MS (ESI) m/z 505 (M+H)⁺.

### Example 102: (2R,4R)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide (Compound 201)

The title compound was prepared using the method described for the synthesis of Example 101B. It was the first of two stereoisomers to elute during the SFC purification step (18 mg, 10%). ¹H NMR (500 MHz, DMSO-d₆) δ ppm 9.14 (d, J=2.0 Hz, 1H), 8.93 (s, 1H), 8.36 (dd, J=8.0, 2.0 Hz, 1H), 8.13 (d, J=1.5 Hz, 1H), 7.95-7.88 (m, 2H), 7.42-7.38 (m, 1H), 7.22 (dd, J=8.5, 2.5 Hz, 1H), 6.90 (d, J=8.5 Hz, 1H), 5.78-5.69 (m, 1H), 4.86-4.80 (m, 1H), 4.67 (dd, J=12.0, 2.5 Hz, 1H), 2.58 (s, 6H), 2.42-2.34 (m, 1H), 1.78-1.69 (m, 1H); MS (ESI) m/z 505 (M+H)⁺.

### Example 103: (2R,4R)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide (Compound 202)

The title compound was prepared using the procedures described for the synthesis of Example 131D, substituting the product from Example 100A for the product from Example 73B. The crude product was purified by chiral SFC separation [Column: Chiralpak® IG, 10×250 mm, 5 μm, gradient: 40% methanol in CO₂ (isocratic), flow rate: 15 g/minute; column temperature: 40° C.; automatic back-pressure regulator setting: 1700 psi] to give the title compound as the earlier eluting fraction. ¹H NMR (500 MHz, methanol-d₄) δ ppm 7.93-7.85 (m, 2H), 7.69-7.62 (m, 1H), 7.45-7.41 (m, 1H), 7.16 (dd, J=8.5, 2.5 Hz, 1H), 6.93 (d, J=8.5 Hz, 1H), 4.96-4.89 (m, 1H), 4.64 (dd, J=11.5, 2.5 Hz, 1H), 2.68 (s, 6H), 2.59-2.51 (m, 1H), 1.95-1.85 (m, 1H); MS (ESI) m/z 488 (M−H)⁻.

### Example 104: (2R,4R)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide (Compound 203)

### Example 104A: (2R)-6-chloro-4-oxo-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide

The methodologies described in Example 30D substituting the product of Example 1B for 3-(2-(4-chloro-3-fluorophenoxy)acetamido)bicyclo[1.1.1]pentane-1-carboxylic acid and substituting Example 68C for Example 30C gave the title intermediate. MS (APCI⁺) m/z 540 (M+H)⁺.

### Example 104B: (2R,4R)-6-chloro-4-hydroxy-N-(4-{5-[cis-3-(trifluoroethoxy)cyclobutyl]-1,3,4-oxadiazol-2-yl}bicyclo[2.2.2]octan-1-yl)-3,4-dihydro-2H-1-benzopyran-2-carboxamide

The methodologies described in Example 5 substituting Example 104A for Example 4 gave the title compound. ¹H NMR (500 MHz, DMSO-d₆) δ ppm 7.43 (s, 1H), 7.38 (dd, J=2.8, 1.0 Hz, 1H), 7.18 (dd, J=8.8, 2.7 Hz, 1H), 6.87 (d, J=8.7 Hz, 1H), 4.89 (p, J=7.5 Hz, 1H), 4.78 (dd, J=10.7, 6.0 Hz, 1H), 4.58 (dd, J=11.8, 2.2 Hz, 1H), 2.82 (tdt, J=9.7, 7.4, 2.3 Hz, 2H), 2.47 (ddd, J=9.9, 7.5, 2.6 Hz, 2H), 2.28 (ddd, J=12.9, 5.9, 2.3 Hz, 1H), 1.96 (s, 12H), 1.80-1.70 (m, 1H); MS (APCI⁺) m/z 542 (M+H)⁺.

### Example 105: (2S,4S)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide (Compound 204)

### Example 105A: (2S)-6-chloro-4-oxo-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide

The methodologies described in Example 30D substituting the product of Example 10A for 3-(2-(4-chloro-3-fluorophenoxy)acetamido)bicyclo[1.1.1]pentane-1-carboxylic acid and substituting Example 68C for Example 30C gave the title intermediate. MS (APCI⁺) m/z 540 (M+H)⁺.

### Example 105B: (2S,4S)-6-chloro-4-hydroxy-N-(4-{5-[cis-3-(trifluoromethoxy)cyclobutyl]-1,3,4-oxadiazol-2-yl}bicyclo[2.2.2]octan-1-yl)-3,4-dihydro-2H-1-benzopyran-2-carboxamide

The methodologies described in Example 5 substituting Example 105A for Example 4 gave the title compound. ¹H NMR (400 MHz, DMSO-d₆) δ ppm 7.41 (s, 1H), 7.40-7.35 (m, 1H), 7.18 (dd, J=8.7, 2.7 Hz, 1H), 6.87 (d, J=8.7 Hz, 1H), 5.67 (s, 1H), 4.89 (p, J=7.5 Hz, 1H), 4.78 (dd, J=10.5, 6.0 Hz, 1H), 4.58 (dd, J=11.8, 2.3 Hz, 1H), 2.82 (dtt, J=9.7, 7.4, 2.5 Hz, 2H), 2.54-2.42 (m, 2H), 2.28 (ddd, J=12.9, 5.9, 2.3 Hz, 1H), 1.96 (s, 12H), 1.75 (dt, J=12.7, 11.0 Hz, 1H); MS (APCI⁺) m/z 542 (M+H)⁺.

### Example 106: (2R,4R)-6-chloro-4-hydroxy-N-(1-{[cis-3-(trifluoromethoxy)cyclobutyl]carbamoyl}-2-oxabicyclo[2.2.2]octan-4-yl)-3,4-dihydro-2H-1-benzopyran-2-carboxamide (Compound 205)

### Example 106A: cis-3-(trifluoromethoxy)cyclobutanamine

A mixture of the product of Example 250 (1.25 g, 6.79 mmol), N,N-diisopropylethylamine (3.56 mL, 20.37 mmol) and 2-(trimethylsilyl)ethanol (9.73 mL, 67.9 mmol) in toluene (20 mL) was stirred at ambient temperature and diphenylphosphoryl azide (2.80 g, 10.18 mmol) was added. The mixture was heated to 80° C. overnight, then cooled to ambient temperature. The solution was diluted with toluene (30 mL) and washed with water (5 0 mL), saturated NaHCO₃ (50 mL) and brine (50 mL). The organic fraction was dried with magnesium sulfate and filtered. The filtrate was concentrated and purified on silica gel using a gradient of 0-30% ethyl acetate in heptane to give 1.57 g of tert-butyl (cis-3-(trifluoromethoxy)cyclobutyl)carbamate. This compound was dissolved in dichloromethane (20 mL) and treated with 13 mL of trifluoroacetic acid for 3 hours. Solvent and excess trifluoroacetic acid were removed under high vacuum to give 1.8 g of the title compound, which was used without further purification. ¹H NMR (600 MHz, DMSO-d₆) δ ppm 8.13 (s, 3H), 4.65 (p, J=7.2 Hz, 1H), 3.37 (s, 1H), 2.71 (tdt, J=9.5, 7.0, 2.4 Hz, 2H), 2.38-2.29 (m, 2H).

### Example 106B: Tert-Butyl (1-((cis-3-(trifluoromethoxy)cyclobutyl)carbamoyl)-2-oxabicyclo[2.2.2]octan-4-yl)carbamate

A mixture of the product of Example 64C (0.1 g, 0.369 mmol), the product of Example 106A (0.150 g, 0.461 mmol), N-ethyl-N-isopropylpropan-2-amine (0.322 mL, 1.843 mmol) and 2-(3H-[1,2,3]triazolo[4,5-b]pyridin-3-yl)-1,1,3,3-tetramethylisouronium hexafluorophosphate(V) (0.210 g, 0.553 mmol) in N,N-dimethylformamide (5.0 mL) was stirred at ambient temperature for 16 hours. Solvent was removed under high vacuum and the residue was purified by HPLC (Phenomenex® Luna® C18(2) 10 μm 100 Å AXIA™ column (250 mm×50 mm). A 35-100% gradient of acetonitrile (A) and 0.1% trifluoroacetic acid in water (B) was used over 25 minutes, at a flow rate of 50 mL/minute) to give 118 mg of the title compound. ¹H NMR (600 MHz, DMSO-d₆) δ ppm 7.96 (d, J=8.4 Hz, 1H), 6.69 (s, 1H), 4.52 (p, J=7.3 Hz, 1H), 3.94-3.84 (m, 1H), 3.89 (s, 2H), 2.57 (tdt, J=9.7, 6.9, 2.6 Hz, 2H), 2.31 (ddd, J=11.7, 10.1, 5.9 Hz, 2H), 1.96-1.88 (m, 4H), 1.80-1.71 (m, 4H), 1.36 (s, 9H).

### Example 106C: 4-amino-N-((cis)-3-(trifluoromethoxy)cyclobutyl)-2-oxabicyclo[2.2.2]octane-1-carboxamide Trifluoroacetic Acid

A mixture of the product of Example 106B (0.12 g, 0.294 mmol) and 2,2,2-trifluoroacetic acid (0.023 mL, 0.294 mmol) in dichloromethane (5 mL) was stirred at ambient temperature for 16 hours. Solvent and excess 2,2,2-trifluoroacetic acid were removed under high vacuum to give 118 mg of the title compound. ¹H NMR (600 MHz, DMSO-d₆) δ ppm 8.20 (s, 3H), 8.03 (d, J=8.3 Hz, 1H), 4.53 (p, J=7.3 Hz, 1H), 3.98-3.85 (m, 1H), 3.85 (s, 2H), 2.59 (dtd, J=9.7, 7.0, 3.1 Hz, 2H), 2.30 (dt, J=11.9, 8.8 Hz, 2H), 2.00 (td, J=12.6, 12.1, 8.7 Hz, 2H), 1.86 (tt, J=11.5, 7.3 Hz, 6H).

### Example 106D: (2R,4R)-6-chloro-4-hydroxy-N-(1-{[cis-3-(trifluoromethoxy)cyclobutyl]carbamoyl}-2-oxabicyclo[2.2.2]octan-4-yl)-3,4-dihydro-2H-1-benzopyran-2-carboxamide

The title compound was synthesized using the same procedures as described in Example 87A through Example 87B substituting the product of Example 86G with the product of Example 106C and the product of Example 10A with the product of Example 1B. ¹H NMR (400 MHz, DMSO-d₆) δ ppm 8.00 (d, J=8.4 Hz, 1H), 7.58 (s, 1H), 7.38 (d, J=2.7 Hz, 1H), 7.18 (dd, J=8.7, 2.7 Hz, 1H), 6.86 (d, J=8.7 Hz, 1H), 5.54 (s, 1H), 4.78 (dd, J=10.6, 5.9 Hz, 1H), 4.62-4.47 (m, 2H), 4.08-3.98 (m, 2H), 3.96-3.81 (m, 1H), 2.59 (dd, J=11.8, 3.1 Hz, 2H), 2.38-2.23 (m, 3H), 2.06 (dddd, J=17.4, 10.6, 6.0, 3.4 Hz, 2H), 1.94 (ddd, J=18.5, 11.4, 3.1 Hz, 3H), 1.91-1.77 (m, 2H), 1.80-1.69 (m, 1H); MS (APCI⁺) m/z 519.06 (M+H)⁺.

### Example 107: (2S,4S)-6-chloro-4-hydroxy-N-(1-{[cis-3-(trifluoromethoxy)cyclobutyl]carbamoyl}-2-oxabicyclo[2.2.2]octan-4-yl)-3,4-dihydro-2H-1-benzopyran-2-carboxamide (Compound 206)

The title compound was synthesized using the same procedures as described in Example 87A through Example 87B substituting the product of Example 86G with Example 106C. ¹H NMR (500 MHz, DMSO-d₆) δ ppm 8.02 (d, J=8.4 Hz. 1H), 7.60 (s, 1H), 7.37 (dd, J=2.8, 0.9 Hz, 1H), 7.18 (dd, J=8.6, 2.6 Hz, 1H), 6.87 (d, J=8.7 Hz, 1H), 5.67 (s, 1H), 4.78 (dd, J=10.6, 5.9 Hz, 1H), 4.62-4.48 (m, 2H), 4.03 (qd, J=7.9, 2.5 Hz, 2H), 3.91 (dtd, J=16.2, 9.1, 7.3 Hz, 1H), 2.58 (tdd, J=12.0, 8.7, 5.2 Hz, 2H), 2.37-2.23 (m, 3H), 2.12-1.86 (m, 6H), 1.89-1.77 (m, 2H), 1.75 (ddd, J=12.9, 10.8, 9.7 Hz, 1H); MS (APCI⁺) m/z 519.06 (M+H)⁺.

### Example 108: (2R,4R)-6-chloro-N-{trans-4-[3-(4-chloro-3-fluorophenyl)-2-oxoimidazolidin-1-yl]cyclohexyl}-4-hydroxy-3,4-dihydro-2H-1-benzopyran-2-carboxamide (Compound 207)

### Example 108A: Benzyl ((trans)-4-(3-(4-chloro-3-fluorophenyl)-2-oxoimidazolidin-1-yl)cyclohexyl)carbamate

4-Chloro-3-fluoroiodobenzene (161 mg, 0.63 mmol), tris(dibenzylideneacetone)dipalladium(O) (24.0 mg, 0.026 mmol), 2-(dicyclohexylphosphino)-2′,4′,6′-triisopropylbiphenyl (24.9 mg, 0.052 mmol, XPhos), the product of Example 37C (166 mg, 0.52 mmol) and cesium carbonate (426 mg, 1.31 mmol) were suspended in dioxane (5 mL). The reactor was degassed three times with a nitrogen back flush each time and then sealed. The reaction mixture was warmed to 100° C. and stirred for 2 hours. The resulting mixture was cooled to ambient temperature and combined with diatomaceous earth (about 5 grams) and concentrated under reduced pressure to a free flowing powder. The powder was directly purified by reversed-phase flash chromatography [Custom packed YMC TriArt™ C18 Hybrid 20 μm column, 25×150 mm, flow rate 70 mL/minute, 5-100% gradient of acetonitrile in buffer (0.025 M aqueous ammonium bicarbonate, adjusted to pH 10 with ammonium hydroxide)] to give the title compound (180 mg, 0.41 mmol, 77% yield). ¹H NMR (400 MHz, DMSO-d₆) δ ppm 7.73 (dd, J=12.7, 2.5 Hz, 1H), 7.49 (t, J=8.8 Hz, 1H), 7.40-7.27 (m, 5H), 7.21 (d, J=7.9 Hz, 1H), 5.01 (s, 2H), 3.77 (dd, J=9.4, 6.7 Hz, 2H), 3.63-3.53 (m, 1H), 3.43 (dd, J=9.3, 6.7 Hz, 2H), 3.32-3.23 (m, 2H), 1.92-1.84 (m, 2H), 1.69-1.61 (m, 2H), 1.60-1.50 (m, 2H), 1.30 (qd, J=12.6, 3.8 Hz, 2H); MS (APCI⁺) ml 466 (M+H)⁺.

### Example 108B: (2R,4R)-6-chloro-N-{trans-4-[3-(4-chloro-3-fluorophenyl)-2-oxoimidazolidin-1-yl]cyclohexyl}-4-hydroxy-3,4-dihydro-2H-1-benzopyran-2-carboxamide

The reaction and purification conditions described in Example 3C substituting the product of Example 108A for the product of Example 3A, and also raising the reaction temperature for the first step from ambient temperature in trifluoroacetic acid to 65° C. in trifluoroacetic acid gave the title compound. ¹H NMR (400 MHz, DMSO-d₆) δ ppm 7.91 (d, J=8.2 Hz, 1H), 7.74 (dd, J=12.7, 2.6 Hz, 1H), 7.49 (t, J=8.8 Hz, 1H), 7.39 (dd, J=2.8, 1.0 Hz, 1H), 7.33 (ddd, J=9.0, 2.6, 1.0 Hz, 1H), 7.20 (dd, J=8.8, 2.6 Hz, 1H), 6.89 (d, J=8.7 Hz, 1H), 5.69 (d, J=6.4 Hz, 1H), 4.82 (dt, J=11.4, 5.9 Hz, 1H), 4.62 (dd, J=11.9, 2.2 Hz, 1H), 3.78 (dd, J=9.4, 6.6 Hz, 2H), 3.69-3.57 (m, 2H), 3.50-3.41 (m, 2H), 2.35 (ddd, J=12.8, 5.9, 2.3 Hz, 1H), 1.89-1.81 (m, 2H), 1.78-1.53 (m, 5H), 1.53-1.40 (m, 2H); MS (APCI⁺) m/z 504 (M−H₂O+H)⁺.

### Example 109: (2S,4R)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide (Compound 208)

### Example 109A: Tert-Butyl (3-(2-(trans-3-(trifluoromethoxy)cyclobutoxy)acetamido)bicyclo[1.1.1]pentan-1-yl)carbamate

The reaction and purification conditions described in Example 2B substituting tert-butyl (3-aminobicyclo[1.1.1]pentan-1-yl)carbamate (PharmaBlock) for the product of Example 2A and the product of Example 250 for the product of Example 1B gave the title compound. ¹H NMR (500 MHz, DMSO-d₆) δ ppm 8.31 (s, 1H), 7.51 (s, 1H), 4.48 (p, J=7.1 Hz, 1H), 3.73-3.64 (m, 3H), 2.77-2.68 (m, 2H), 2.18-2.11 (m, 2H), 2.11 (s, 6H), 1.37 (s, 9H); MS (APCI⁺) m/z 395 (M+H)⁺.

### Example 109B: (2S,4R)-6-chloro-4-hydroxy-N-[3-(2-{cis-3-(trifluoromethoxy)cyclobutyl]oxy}acetamido)bicyclo[1.1.1]pentan-1-yl-3,4-dihydro-2H-1-benzopyran-2-carboxamide

Trifluoroacetic acid (0.5 mL) was added to the product of Example 109A (32.6 mg, 0.083 mmol), and the reaction was stirred at ambient temperature for 15 minutes. The resulting solution was concentrated under reduced pressure to a residue. Triethylamine (0.058 mL), N,N-dimethylformamide (1 mL), the product of Example 73B (20.8 mg, 0.091 mmol) and 1-[bis(dimethylamino)methylene]-1H-1,2,3-triazolo[4,5-b]pyridinium 3-oxid hexafluorophosphate (33.3 mg, 0.088 mmol, HATU) were added in sequential order. The resulting reaction mixture was stirred at ambient temperature for 1 hour. Water (0.2 mL) was then added. The resulting solution was filtered through a glass microfiber frit and directly purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (32 mg, 0.063 mmol, 77% yield). ¹H NMR (500 MHz, DMSO-d₆) δ ppm 8.72 (s, 1H), 8.37 (s, 1H), 7.31 (d, J=2.7 Hz, 1H), 7.25 (dd, J=8.7, 2.7 Hz, 1H), 6.93 (d, J=8.8 Hz, 1H), 5.64-5.60 (m, 1H), 4.60-4.56 (m, 1H), 4.54 (dd, J=10.9, 2.7 Hz, 1H), 4.48 (p, J=7.2 Hz, 1H), 3.73 (s, 2H), 3.72-3.65 (m, 1H), 2.78-2.69 (m, 2H), 2.27-2.23 (m, 6H), 2.18-2.11 (m, 2H), 2.08 (ddd, J=13.9, 3.8, 2.8 Hz, 1H), 1.94-1.84 (m, 1H); MS (APCI⁺) m/z 505 (M−H₂O+H)⁺.

### Example 110: (2R)-6-chloro-N-{3-[4-(4-chlorophenyl)-1H-pyrazol-1-yl]bicyclo[1.1.1]pentan-1-yl}-4-oxo-3,4-dihydro-2H-1-benzopyran-2-carboxamide (Compound 209)

### Example 110A: Methyl 3-(4-(4-chlorophenyl)-1H-pyrazol-1-yl)bicyclo[1.1.1]pentane-1-carboxylate

A 30 mL vial was charged with iodomesitylene diacetate (243 mg, 0.667 mmol), 3-(methoxycarbonyl)bicyclo[1.1.1]pentane-1-carboxylic acid (227 mg, 1.33 mmol, Synthonix) and toluene (5 mL). The mixture was stirred at 60° C. for 45 minutes. Toluene was then removed under high vacuum. Iridium(III) bis[2-(2,4-difluorophenyl)-5-methylpyridine-N,C₂₀]-4,40-di-tert-butyl-2,20-bipyridine hexafluorophosphate (25 mg, 0.025 mmol), 4-(4-chlorophenyl)-1H-pyrazole (240 mg, 1.34 mmol, Matrix), 4,7-diphenyl-1,10-phenanthroline (120 mg, 0.361 mmol), copper(II) acetate (121 mg, 0.666 mmol), 2-tert-butyl-1,1,3,3-tetramethylguanidine (BTMG, 0.48 mL, 2.38 mmol) were added sequentially followed by dioxane (5.0 mL). The vial was degassed by sparging with nitrogen for 3 minutes before sealing with a polytetrafluoroethylene-lined cap. The vial was then put inside a 250 mL glass Dewar filled with water and clamped at a 45 angle to increase exposure to the light-emitting diode (LED). (The glass Dewar was used to focus the blue LED to the vial, and the water bath was used to keep a constant temperature). The reaction was stirred and irradiated using an 18W 450 nm HepatoChem blue LED photoredox lamp just 5 cm above the vial. The bath temperature was measured as 22° C. when setting up the reaction and rose to 30° C. after an hour, and the temperature was stabilized at 30° C. for the remainder of the reaction time. After 18 hours, the reaction mixture was quenched by exposing to air and partitioned between water (50 mL) and dichloromethane (2×50 mL). The organic layers were combined and dried over sodium sulfate and concentrated under reduced pressure. The residue was taken up in methanol (5 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (40 mg, 0.13 mmol, 9.8% yield). ¹H NMR (400 MHz, DMSO-d₆) δ ppm 1H NMR (400 MHz, DMSO-d₆) δ ppm 8.34 (d, J=0.9 Hz, 1H), 7.98 (d, J=0.9 Hz, 1H), 7.67-7.60 (m, 2H), 7.45-7.35 (m, 2H), 3.68 (s, 3H), 2.52 (s, 6H); MS (APCI⁺) m/z 303 (M+H)⁺.

### Example 110B: 3-(4-(4-chlorphenyl)-1H-pyrazol-1-yl)bicyclo[1.1.1]pentane-1-carboxylic Acid

The product of Example 110A (35 mg, 0.116 mmol) was combined with methanol (5 mL) and stirred at ambient temperature. Aqueous NaOH (0.185 mL, 2.5 M) was added. After stirring for 30 minutes, more NaOH (0.23 mL, 2.5 M) was added and the resulting solution was stirred at 45° C. for 2 hours and then at ambient temperature for 18 hours. The reaction mixture was combined with diatomaceous earth (about 5 grams) and concentrated under reduced pressure to a free flowing powder. The powder was directly purified by reversed-phase flash chromatography [Custom packed YMC TriArt™ C18 Hybrid 20 μm column, 25×150 mm, flow rate 70 mL/minute, 5-100% gradient of acetonitrile in buffer (0.1% trifluoroacetic acid)] to give the title compound (32 mg, 0.11 mmol, 96% yield). MS (APCI⁺) m/z 289 (M+H)⁺.

### Example 110C: 3-(4-(4-chlorophenyl)-1H-pyrazol-1-yl)bicyclo[1.1.1]pentan-1-amine

A mixture of the product of Example 110B (35 mg, 0.12 mmol), N,N-diisopropylethylamine (0.064 mL, 0.36 mmol), and 2-(trimethylsilyl)ethanol (0.26 mL, 1.82 mmol) in toluene (2 mL) was stirred at ambient temperature and diphenylphosphoryl azide (0.039 mL, 0.182 mmol) was added. The mixture was heated at 55° C. for 18 hours, cooled to ambient temperature, and then concentrated under reduced pressure. Trifluoroacetic acid (1.0 mL) was added to the residue. The mixture was stirred at ambient temperature for 1 hour and then concentrated under reduced pressure. The resulting residue was taken up in methanol (3 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (20 mg, 0.062 mmol, 51% yield). MS (APCI⁺) m/z 260 (M+H)⁺.

### Example 110D: (2R)-6-chloro-N-{3-[4-(4-chlorophenyl)-1H-pyrazol-1-yl]bicyclo[1.1.1]pentan-1-yl}-4-oxo-3,4-dihydro-2H-1-benzopyran-2-carboxamide

The reaction and purification conditions described in Example 2B substituting the product of Example 110C for the product of Example 2A gave the title compound. ¹H NMR (500 MHz, DMSO-d₆) δ ppm 9.18 (s, 1H), 8.30 (d, J=0.8 Hz, 1H), 7.96 (d, J=0.8 Hz, 1H), 7.68-7.60 (m, 4H), 7.42-7.38 (m, 2H), 7.19 (d, J=8.5 Hz, 1H), 5.15 (dd, J=8.2, 6.1 Hz, 1H), 3.01-2.93 (m, 2H), 2.51 (s, 6H); MS (APCI⁺) m/z 468 (M+H)⁺.

### Example 111: (2R,4R)-6-chloro-4-hydroxy-N-[(1R*,2S*,4R*,5S*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide (Compound 210)

### Example 111A: rac-(1R,4R)-2,5-diisothiocyanatobicyclo[2.2.1]heptane

To a solution of 2,5-norbornadiene (5.0 g, 54.3 mmol) in toluene (50 mL) was added ammonium thiocyanate (12.4 g, 163 mmol) and a solution of concentrated sulfuric acid (4.63 mL, 87 mmol) in water (3 mL). The resulting reaction mixture was stirred at 75° C. for 36 hours, cooled to ambient temperature, and then diluted with tetrahydrofuran (50 mL). The pH of the mixture was adjusted to around 8 with saturated aqueous ammonium bicarbonate. The organic layer was separated, dried over sodium sulfate, filtered and concentrated under reduced pressure. The residue was purified by silica gel column chromatography (9-33% ethyl acetate in petroleum ether) to give the title compound (1.8 g, 8.56 mmol, 16% yield). ¹H NMR (400 MHz, CDCl₃) δ ppm 3.54 (dd, J=3.1, 7.1 Hz, 2H), 2.60 (br d, J=4.4 Hz, 2H), 1.80-1.66 (m, 6H).

### Example 111B: Di-Tert-Butyl rac-(1R,2S,4R,5S)-bicyclo[2.2.1]heptane-2,5-diyldicarbamate

The product of Example 111A (16.0 g, 76 mmol) was combined with dioxane (160 mL) and aqueous HCl (12 M, 160 mL). The reaction was stirred at 100° C. for 12 hours, cooled to ambient temperature, and concentrated under reduced pressure. To the residue was added dichloromethane (300 mL), and the mixture was stirred at 0° C. Di-tert-butyl dicarbonate (88 ml, 380 mmol) was slowly added. The ice bath was then removed, and the resulting reaction mixture was allowed to stir at ambient temperature for 13 hours. The resulting organic mixture was washed with 0.5 M aqueous HCl (8×100 mL), dried over sodium sulfate and triturated with petroleum ether (200 mL) to give the title compound (6 g, 17.46 mmol, 23% yield). ¹H NMR (400 MHz, CDCl₃) δ ppm 6.73 (br d, J=6.5 Hz, 2H), 3.25-3.08 (m, 2H), 1.96 (br s, 2H), 1.50-1.42 (m, 2H), 1.37 (s, 18H), 1.29 (br s, 2H), 1.20 (br d, J=12.5 Hz, 2H).

### Example 111C: rac-(1R,2S,4R,5S)-bicyclo[2.2.1]heptane-2,5-diamine, 2 Hydrochloric Acid

To a solution of the product of Example 111B (2 g, 6.13 mmol) in dichloromethane (50 mL) stirred at 0° C. was added HCl (4.0 M HCl in methanol, 20 mL). The ice bath was removed and the reaction solution was allowed to stir at 25° C. for 13 hours and then concentrated under reduced pressure to give the title compound (1.1 g, 5.52 mmol, 90% yield). ¹H NMR (400 MHz, methanol-d₄) δ ppm S=3.22 (br dd, J=3.5, 7.7 Hz, 2H), 2.56 (br d, J=4.2 Hz, 2H), 1.98-1.88 (m, 2H), 1.79 (s, 2H), 1.60 (td, J=4.4, 14.0 Hz, 2H); MS (ESI⁺) m/z 127 (M+H)⁺.

### Example 111D: Benzyl [rac-(1R,2S,4R,5S)-5-aminobicyclo[2.2.1]heptan-2-yl]carbamate

To a solution of the product of Example 111C (37.5 g, 188 mmol) in a solvent mixture of dichloromethane (1200 mL) and methanol (400 mL) stirred at 0° C. was added N,N-diisopropylethylamine (132 mL, 753 mmol). The reaction solution was stirred at 0° C. for 1 hour. Then a solution of benzyl chloroformate (12.85 g, 75 mmol) in dichloromethane (400 mL) was added dropwise at 0° C. The reaction mixture was allowed to warm to 25° C. and stirred at 25° C. for 13 hours. Hydrochloric acid (4.0 M in methanol) was added to the reaction to adjust the pH to 3. The reaction mixture was then concentrated under reduced pressure, taken up in water (1.0 L) and then extracted with ethyl acetate (4×400 mL). The pH of the aqueous phase was adjusted to 9 with potassium carbonate and then extracted with dichloromethane (4×400 mL). The organic layers were combined and concentrated under reduce pressure. The resulting residue was purified by silica gel column chromatography (dichloromethane:methanol 50:1 to 10:1, 0.5% NH₃) to give the title compound (35.3 g, 136 mmol, 18% yield). MS (ESI⁺) m/z 261 (M+H)⁺.

### Example 111E: Benzyl ((1RS,2SR,4RS,5SR)-5-((R)-6-chloro-4-oxochroman-2-carboxamido)bicyclo[2.2.1]heptan-2-yl)carbamate

The reaction and purification conditions described in Example 2B substituting the product of Example 111D for the product of Example 2A gave the title compound. MS (APCI⁺) m/z 469 (M+H)⁺.

### Example 111F: Benzyl [(1R*,2S*,4R*,5S*)-5-{[(2R)-6-chloro-4-oxo-3,4-dihydro-2H-1-benzopyran-2-carbonyl]amino}bicyclo[2.2.1]heptan-2-yl]carbamate

The product of Example 111E was purified by preparative chiral HPLC [CHIRALCEL® OJ 20 μm column, 20×250 mm, flow rate 7.5 mL/minute, 40% ethanol and 5% 2-propanol in heptane (isocratic gradient)]. The earlier eluting fraction was collected and concentrated to give the title compound. MS (APCI⁺) m/z 469 (M+H)⁺.

### Example 111G: (2R)-6-chloro-4-oxo-N-[(1R*,2S*,4R*,5S*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide

The reaction and purification conditions described in Example 1C substituting the product of Example 111F for the product of Example 1A, the product of Example 13P for the product of Example 1B, and also raising the reaction temperature for the first step from ambient temperature in trifluoroacetic acid to 70° C. in trifluoroacetic acid gave the title compound. MS (APCI⁺) m/z 469 (M+H)⁺.

### Example 111H: (2R,4R)-6-chloro-4-hydroxy-N-[(1R*,2S*,4R*,5S*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide

The reaction and purification conditions described in Example 6C substituting the product of Example 111G for the product of Example 6B gave the title compound. ¹H NMR (500 MHz, DMSO-d₆) δ ppm 7.84 (d, J=6.9 Hz, 1H), 7.57 (d, J=7.0 Hz, 1H), 7.37 (d, J=2.2 Hz, 1H), 7.18 (dd, J=8.7, 2.7 Hz, 1H), 6.88 (d, J=8.7 Hz, 1H), 5.72 (br s, 1H), 4.79 (dd, J=10.7, 6.0 Hz, 1H), 4.59 (dd, J=11.9, 2.3 Hz, 1H), 4.47 (p, J=7.1 Hz, 1H), 3.74 (s, 2H), 3.69 (p, J=6.9 Hz, 1H), 3.55-3.48 (m, 2H), 2.77-2.68 (m, 2H), 2.29 (ddd, J=12.9, 6.0, 2.4 Hz, 1H), 2.18-2.05 (m, 4H), 1.80-1.69 (m, 1H), 1.65-1.54 (m, 2H), 1.44-1.32 (m, 4H); MS (APCI⁺) m/z 515 (M−H₂O+H)⁺.

### Example 112: (2R,4R)-6-chloro-4-hydroxy-N-[(1S*,2R*,4S*,5R*)-5-(2-{[cis-3-(trifluoromethoxy)cyclobutyl]oxy}acetamido)bicyclo[2.2.1]heptan-2-yl]-3,4-dihydro-2H-1-benzopyran-2-carboxamide (Compound 211)

### Example 112A: Benzyl [(1S*,2R*,4S*,5R*)-5-{[(2R)-6-chloro-4-oxo-3,4-dihydro-2H-1-benzopyran-2-carbonyl]amino}bicyclo[2.2.1]heptan-2-yl]carbamate

The product of Example 111E was purified by preparative chiral HPLC [CHIRALCEL® OJ 20 μm column, 20×250 mm, flow rate 7.5 mL/minute, 40% ethanol and 5% 2-propanol in heptane (isocratic gradient)]. The later eluting fraction was collected and concentrated to give the title compound. MS (APCI⁺) m/z 469 (M+H)⁺.

### Example 112B: (2R)-6-chloro-4-oxo-N-[(1S*,2R*,4S*,5R*)-5-(2-{cis-3-(trifluoromethoxy)cyclobutyl]oxy}acetamido)bicyclo[2.2.1]heptan-2-yl]-3,4-dihydro-2H-1-benzopyran-2-carboxamide

The reaction and purification conditions described in Example 1C substituting the product of Example 112A for the product of Example 1A, the product of Example 13P for the product of Example 1B, and also raising the reaction temperature for the first step from ambient temperature in trifluoroacetic acid to 70° C. in trifluoroacetic acid gave the title compound. MS (APCI⁺) m/z 469 (M+H)⁺.

### Example 112C: (2R,4R)-6-chloro-4-hydroxy-N-[(1S*,2R*,4S*,5R*)-5-(2-{[cis-3-(trifluoromethoxy)cyclobutyl]oxy}acetamido)bicyclo[2.2.]heptan-2-yl]-3,4-dihydro-2H-1-benzopyran-2-carboxamide

The reaction and purification conditions described in Example 6C substituting the product of Example 112B for the product of Example 6B gave the title compound. ¹H NMR (500 MHz, DMSO-d₆) δ ppm 7.84 (d, J=7.0 Hz, 1H), 7.57 (d, J=7.0 Hz, 1H), 7.37 (dd, J=2.8, 1.0 Hz, 1H), 7.18 (dd, J=8.7, 2.6 Hz, 1H), 6.88 (d, J=8.8 Hz, 1H), 5.72 (br s, 1H), 4.79 (dd, J=10.7, 5.9 Hz, 1H), 4.60 (dd, J=11.8, 2.2 Hz, 1H), 4.47 (p, J=7.2 Hz, 1H), 3.74 (s, 2H), 3.70 (t, 0.1=6.8 Hz, 1H), 3.56-3.48 (m, 2H), 2.78-2.68 (m, 2H), 2.30 (ddd, J=12.9, 5.9, 2.3 Hz, 1H), 2.19-2.05 (m, 4H), 1.79-1.69 (m, 1H), 1.64-1.55 (m, 2H), 1.45-1.31 (m, 4H); MS (APCI⁺) m/z 515 (M−H₂O+H)⁺.

### Example 113: (2R,4R)-6-chloro-N-{3-[4-(4-chlorophenyl)-1H-pyrazol-1-yl]bicyclo[1.1.1]pentan-1-yl}-4-hydroxy-3,4-dihydro-2H-1-benzopyran-2-carboxamide (Compound 212)

The reaction and purification conditions described in Example 6C substituting the product of Example 110 for the product of Example 6B gave the title compound. ¹H NMR (600 MHz, DMSO-d₆) δ ppm 8.93 (s, 1H), 8.31 (d, J=0.8 Hz, 1H), 7.96 (d, J=0.8 Hz, 1H), 7.65-7.60 (m, 2H), 7.42-7.36 (m, 3H), 7.21 (dd, J=8.7, 2.7 Hz, 1H), 6.90 (d, J=8.7 Hz, 1H), 5.76 (br s, 1H), 4.83 (dd, J=10.7, 5.9 Hz, 1H), 4.65 (dd, J=12.0, 2.3 Hz, 1H), 2.54 (s, 6H), 2.39 (ddd, J=12.9, 5.8, 2.4 Hz, 1H), 1.77-1.68 (m, 1H); MS (APCI⁺) m/z 470 (M+H)⁺.

### Example 114: (2R)-6-chloro-4-oxo-N-[trans-4-(2-{[cis-3-(trifluoromethoxy)cyclobutyl]oxy}acetamido)cyclohexyl]-3,4-dihydro-2H-1-benzopyran-2-carboxamide (Compound 213)

### Example 114A: (2R)—N-(trans-4-aminocyclohexyl)-6-chloro-4-oxo-3,4-dihydro-2H-1-benzopyran-2-carboxamide

Benzyl (trans-4-aminocyclohexyl)carbamate (30 mg, 0.12 mmol) was combined with the product of Example 1B (27.4 mg, 0.12 mmol), triethylamine (0.084 mL) and N,N-dimethylformamide (2 mL). The mixture was stirred at ambient temperature and 1-[bis(dimethylamino)methylene]-1H-1,2,3-triazolo[4,5-b]pyridinium 3-oxid hexafluorophosphate (55 mg, 0.145 mmol, HATU) was added. The resulting suspension was stirred at ambient temperature for 1 hour and then concentrated under reduced pressure. Trifluoroacetic acid (0.5 mL) was added. The resulting solution was stirred at 65° C. for 30 minutes, cooled to ambient temperature, and then concentrated under reduced pressure. The residue was taken up in methanol (3 mL), filtered through a glass microfiber frit, and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (26 mg, 0.081 mmol, 67% yield). MS (ESI⁺) m/z 323 (M+H)⁺.

### Example 114B: (2R)-6-chloro-4-oxo-N-[trans-4-(2-{[cis-3-(trifluoromethoxy)cyclobutyl]oxy}acetamido)cyclohexyl]-3,4-dihydro-2H-1-benzopyran-2-carboxamide

The reaction and purification conditions described in Example 2B substituting the product of Example 114A for the product of Example 2A, and the product of Example 13P for the product of Example 1B gave the title compound. ¹H NMR (600 MHz, DMSO-d₆) δ ppm 8.17 (d, J=8.0 Hz, 1H), 7.67-7.61 (m, 2H), 7.58 (d, J=8.3 Hz, 1H), 7.17 (dd, J=8.7, 0.5 Hz, 1H), 5.11 (dd, J=8.4, 5.1 Hz, 1H), 4.48 (p, J=7.1 Hz, 1H), 3.75 (s, 2H), 3.70 (tt, J=7.3, 6.4 Hz, 1H), 3.59-3.47 (m, 2H), 3.01-2.90 (m, 2H), 2.77-2.69 (m, 2H), 2.19-2.11 (m, 2H), 1.80-1.65 (m, 4H), 1.39-1.22 (m, 4H); MS (APCI⁺) m/z 519 (M+H)⁺.

### Example 115: (2R,4R)-6-chloro-4-hydroxy-N-[trans-4-(2-{[cis-3-(trifluoromethoxy)cyclobutyl]oxy}acetamido)cyclohexyl]-3,4-dihydro-2H-1-benzopyran-2-carboxamide (Compound 214)

The reaction and purification conditions described in Example 6C substituting the product of Example 114B for the product of Example 6B gave the title compound. ¹H NMR (500 MHz, DMSO-d₆) δ ppm 7.89 (d, J=8.2 Hz, 1H), 7.60 (d, J=8.3 Hz, 1H), 7.38 (dd, J=2.7, 1.0 Hz, 1H), 7.20 (ddd, J=8.7, 2.7, 0.7 Hz, 1H), 6.89 (d, J=8.7 Hz, 1H), 5.70 (d, J=6.3 Hz, 1H), 4.85-4.77 (m, 1H), 4.61 (dd, J=11.9, 2.2 Hz, 1H), 4.48 (p, J=7.1 Hz, 1H), 3.75 (s, 2H), 3.76-3.66 (m, 1H), 3.63-3.51 (m, 2H), 2.78-2.69 (m, 2H), 2.34 (ddd, J=12.9, 5.9, 2.3 Hz, 1H), 2.21-2.11 (m, 2H), 1.80-1.66 (m, 5H), 1.40-1.30 (m, 4H); MS (APCI⁺) m/z 503 (M−H₂O+H)⁺.

### Example 116: (2R,4R)-6-chloro-4-hydroxy-N-[trans-4-{[cis-3-(trifluoromethoxy)cyclobutyl]carbamoyl}cyclohexyl]-3,4-dihydro-2H-1-benzopyran-2-carboxamide (Compound 215)

### Example 116A: Tert-Butyl [trans-4-{[cis-3-(trifluoromethoxy)cyclobutyl]carbamoyl}cyclohexyl]carbamate

The reaction and purification conditions described in Example 2B substituting the product of Example 106A for the product of Example 2A, and trans-4-[(tert-butoxycarbonyl)amino]cyclohexane-1-carboxylic acid for the product of Example 1B gave the title compound. MS (APCI⁺) m/z 325 (M−C(CH₃)₃+H)⁺.

### Example 116B: (2R)-6-chloro-4-oxo-N-[trans-4-{[cis-3-(trifluoromethoxy)cyclobutyl]carbamoyl}cyclohexyl]-3,4-dihydro-2H-1-benzopyran-2-carboxamide

The reaction and purification conditions described in Example 1C substituting the product of Example 116A for the product of Example 1A gave the title compound. MS (APCI⁺) m/z 489 (M+H)⁺.

### Example 116C: (2R,4R)-6-chloro-4-hydroxy-N-[trans-4-{[cis-3-(trifluoromethoxy)cyclobutyl]carbamoyl}cyclohexal]-3,4-dihydro-2H-1-benzopyran-2-carboxamide

The reaction and purification conditions described in Example 6C substituting the product of Example 116B for the product of Example 6B gave the title compound. ¹H NMR (600 MHz, DMSO-d₆) δ ppm 8.06 (d, J=7.9 Hz, 1H), 7.87 (d, J=8.2 Hz, 1H), 7.38 (dd, J=2.7, 1.0 Hz, 1H), 7.19 (ddd, J=8.7, 2.7, 0.8 Hz, 1H), 6.88 (d, J=8.7 Hz, 1H), 5.70 (br s, 1H), 4.81 (dd, J=10.7, 5.9 Hz, 1H), 4.60 (dd, J=12.0, 2.2 Hz, 1H), 4.56 (p, J=7.4 Hz, 1H), 3.93-3.83 (m, 1H), 3.61-3.52 (m, 1H), 2.71-2.62 (m, 2H), 2.34 (ddd, J=12.9, 5.9, 2.3 Hz, 1H), 2.14-2.05 (m, 2H), 1.99 (tt, J=11.9, 3.5 Hz, 1H), 1.83-1.67 (m, 5H), 1.43-1.23 (m, 4H); MS (APCI⁺) m/z 491 (M+H)⁺.

### Example 117: (2R)-6-chloro-4-oxo-N-(3-{[cis-3-(trifluoromethoxy)cyclobutyl]carbamoyl}bicyclo[1.1.1]pentan-1-yl)-3,4-dihydro-2H-1-benzopyran-2-carboxamide (Compound 216)

### Example 117A: (R)-methyl 3-(6-chloro-4-oxochroman-2-carboxamido)bicyclo[1.1.1]pentane-1-carboxylate

The methodologies described in Example 30D substituting the product of Example 1B for 3-(2-(4-chloro-3-fluorophenoxy)acetamido)bicyclo[1.1.1]pentane-1-carboxylic acid and substituting methyl 3-aminobicyclo[1.1.1]pentane-1-carboxylate hydrochloride (Princeton) for Example 30C gave the title intermediate. MS (APCI⁺) m/z 350 (M+H)⁺.

### Example 17B: (R)-3-(6-chloro-4-oxochroman-2-carboxamido)bicyclo[1.1.1]pentane-1-carboxylic Acid

To a solution of Example 117A (0.22 g, 0.64 mmol) in tetrahydrofuran (1.2 mL) was added lithium hydroxide (1 N aqueous, 1.2 mL, 1.2 mmol). The reaction mixture was stirred at ambient temperature for 1 hour, was concentrated, and was neutralized with 1 N HCl. A precipitate formed upon neutralization that was collected by filtration and dried. The title intermediate was impure but carried forward without purification. ¹H NMR (500 MHz, DMSO-d₆) δ ppm 8.89 (s, 1H), 7.66-7.60 (m, 3H), 7.19-7.12 (m, 4H), 6.89 (d, J=8.6 Hz, 2H), 5.06 (t, J=7.1 Hz, 1H), 2.94 (d, J=7.2 Hz, 2H), 2.06 (s, 6H); MS (APCI⁺) m/z 336 (M+H)⁺.

### Example 117C: (2R)-6-chloro-4-oxo-N-(3-{[cis-3-(trifluoromethoxy)cyclobutyl]carbamoyl}bicyclo[1.1.1]pentan-1-yl)-3,4-dihydro-2H-1-benzopyran-2-carboxamide

The methodologies described in Example 30D substituting Example 117B for 3-(2-(4-chloro-3-fluorophenoxy)acetamido)bicyclo[1.1.1]pentane-1-carboxylic acid and substituting the product of Example 106A for Example 30C gave the title compound. ¹H NMR (500 MHz, DMSO-d₆) δ ppm 8.94 (s, 1H), 8.06 (d, J=8.1 Hz, 1H), 7.68-7.61 (m, 2H), 7.17 (dd, J=8.3, 0.9 Hz, 1H), 5.08 (dd, J=8.9, 5.4 Hz, 1H), 4.56 (t, J=7.2 Hz, 1H), 3.90 (s, 1H), 2.97-2.93 (m, 2H), 2.20 (d, J=9.6 Hz, 2H), 2.15 (s, 6H); MS (APCI⁺) m/z 473 (M+H)⁺.

### Example 118: (2S,4R)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide (Compound 217)

A solution of the product from Example 119G (62 mg, 0.124 mmol) in trifluoroacetic acid (2 mL, 26.0 mmol) was stirred at 0° C. for 5 minutes, and then at room temperature for 3 hours. The solution was concentrated in vacuo, and the residue was dissolved in toluene (3 mL) and concentrated in vacuo (3×). The residue was dissolved in acetonitrile (2 mL), ammonium hydroxide (0.047 mL, 0.124 mmol) was added, and the resulting mixture was stirred at room temperature for 16 hours. The solvent was removed in vacuo to give a mixture of hydroxychromane diastereomers that favored the desired (S,R)-isomer by ˜3:1 by ¹H NMR analysis. The mixture was separated by chiral SFC purification [Column: Chiralpak® IG, 10×250 mm, 5 μm, gradient: 15% methanol in CO₂ (isocratic), flow rate: 15 g/minute; column temperature: 40° C.; automatic back-pressure regulator setting: 1700 psi] to give the title compound (19 mg, 30%). ¹H NMR (500 MHz, DMSO-d₆) δ ppm 8.97 (s, 1H), 7.33 (d, J=2.6 Hz, 1H), 7.27 (dd, J=8.8, 2.7 Hz, 1H), 6.94 (d, J=8.7 Hz, 1H), 5.65 (s, 1H), 4.91 (p, J=7.6 Hz, 1H), 4.64-4.54 (m, 2H), 3.31 (s, 1H), 2.83-2.72 (m, 2H), 2.54 (s, 6H), 2.43 (dt, J=12.4, 9.6 Hz, 2H), 2.12 (dt, J=13.9, 3.3 Hz, 1H), 1.92 (ddd, J=14.2, 11.0, 3.7 Hz, 1H); MS (ESI) m/z 500 (M+H)⁺.

### Example 119: (2S,4S)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide (Compound 218)

### Example 119A: cis-3-hydroxycyclobutanecarbonitrile

3-Oxocyclobutanecarbonitrile (2.0 g, 21.03 mmol) was dissolved in anhydrous tetrahydrofuran (60.0 mL) under a nitrogen atmosphere. The solution was cooled to −78° C. and lithium tri-sec-butylhydroborate (L-Selectride®,1.0 M in tetrahydrofuran, 21.03 mL) was added slowly via syringe. The reaction mixture was stirred at −78° C. for 3 hours. The reaction mixture was quenched with saturated NH₄Cl (250 mL). The mixture was warmed to room temperature and extracted with ethyl acetate (250 mL×3). The organic phases were combined, dried over MgSO₄, filtered and concentrated under reduced pressure to give a residue that was purified by chromatography on silica gel using a solvent gradient of 0-100% ethyl acetate in isohexane to afford the title compound (1.579 g, 15.45 mmol, 73.4% yield). ¹H NMR (500 MHz, DMSO-d₆) δ ppm 5.41 (d, J=7.2 Hz, 1H), 4.05-3.96 (m, 1H), 2.78-2.70 (m, 1H), 2.65-2.51 (m, 2H), 2.14-2.01 (m, 2H).

### Example 119B: cis-3-((tert-butyldiphenylsilyl)oxy)-N′-hydroxycyclobutanecarboximidamide

To the product from Example 119A (0.5 g, 4.89 mmol) and imidazole (0.733 g, 10.76 mmol) in N,N-dimethylformamide (25 mL) at 0° C. was added tert-butyldiphenylchlorosilane (1.382 mL, 5.38 mmol). The reaction mixture was warmed to room temperature and stirred overnight. The reaction mixture was concentrated in vacuo and dissolved in ethyl acetate (50 mL), washed with water (2×50 mL) and brine (50 mL), dried over MgSO₄, filtered and concentrated in vacuo. To a solution of the oil in ethanol (10 mL) was added hydroxylamine (0.790 mL, 12.89 mmol), and the resulting solution was heated at reflux for 16 hours. The reaction mixture was cooled to room temperature and the volatiles were removed in vacuo to give the title compound (1.911 g, 4.93 mmol, 96% yield).

### Example 119C: Tert-Butyl (3-(((cis-3-((tert-butyldiphenylsilyl)oxy)cyclobutyl)(hydroxyimino)methyl)carbamoyl)bicyclo[1.1.1]pentan-1-yl)carbamate

3-((tert-Butoxycarbonyl)amino)bicyclo[1.1.1]pentane-1-carboxylic acid (200 mg, 0.880 mmol) and the product from Example 119B (389 mg, 1.056 mmol) were dissolved in anhydrous N,N-dimethylformamide (5 mL) under a nitrogen atmosphere. The solution was cooled to 0° C., N,N-diisopropylethylamine (0.461 mL, 2.64 mmol) and (1-[bis(dimethylamino)methylene]-1H-1,2,3-triazolo[4,5-b]pyridinium 3-oxid hexafluorophosphate) (HATU, 402 mg, 1.056 mmol) were added, and the reaction mixture was stirred at 0° C. for 10 minutes and then at room temperature for 48 hours. The reaction mixture was diluted with dichloromethane (50 mL) and washed with 1 M HCl (30 mL), saturated aqueous NaHCO₃ (30 mL) and brine (30 mL×3). The organic phase was dried via hydrophobic frit and concentrated in vacuo. The residue was taken up in ethyl acetate (35 mL) and washed with brine (50 mL×3), and the organic phase was dried via hydrophobic frit and concentrated in vacuo. The crude product was purified by column chromatography on silica gel, eluting with a solvent gradient of 0-10% methanol in dichloromethane to afford the title compound (436 mg, 0.709 mmol, 81% yield). ¹H NMR (500 MHz, DMSO-d₆) δ ppm 7.64-7.57 (m, 4H), 7.49-7.41 (m, 6H), 6.25-6.03 (m, 2H), 4.15-4.06 (m, 1H), 2.35-2.25 (m, 3H), 2.22-2.10 (m, 8H), 1.38 (s, 9H), 0.98 (s, 9H).

### Example 119D: Tert-Butyl (3-(3-(cis-3-hydroxycyclobutyl)-1,2,4-oxadiazol-5-yl)bicyclo[1.1.1]pentan-1-yl)carbamate

The product from Example 119C (432 mg, 0.748 mmol) was dissolved in anhydrous tetrahydrofuran (7 mL) under a nitrogen atmosphere and the solution was cooled to 0° C. Tetra-n-butyl ammonium fluoride (1 M in tetrahydrofuran) (2.62 mL, 2.62 mmol) was added slowly via syringe. The reaction mixture was stirred at 0° C. for 15 minutes and then at 60° C. for 6 hours. The reaction mixture was adsorbed onto silica (˜2 g) and purified by chromatography on silica gel using a solvent gradient of 0-10% methanol in dichloromethane to afford the title compound (172 mg, 0.508 mmol, 68.0% yield). ¹H NMR (500 MHz, DMSO-d₆) δ ppm 7.78 (s, 1H), 5.27 (d, J=7.0 Hz, 1H), 4.13-4.03 (m, 1H), 3.06-2.97 (m, 1H), 2.56-2.51 (m, 2H), 2.37 (s, 6H), 2.11-2.01 (m, 2H), 1.39 (s, 9H).

### Example 119L: Tert-Butyl (3-(3-(cis-3-(trifluoromethoxy)cyclobutyl)-1,2,4-oxadiazol-5-yl)bicyclo[1.1.1]pentan-1-yl)carbamate

A mixture of silver(I) trifluoromethanesulfonate (371 mg, 1.445 mmol), potassium fluoride (124 mg, 2.141 mmol) and Selectfluor™ (1-chloromethyl-4-fluoro-1,4-diazoniabicyclo[2.2.2]octane bis(tetrafluoroborate)) (284 mg, 0.803 mmol) was stirred under a nitrogen atmosphere in a flask wrapped with aluminum foil. The flask was cooled in a water bath. The product from Example 119D (172 mg, 0.535 mmol) was dissolved in a mixed solvent of ethyl acetate (3 mL) and tetrahydrofuran (2 mL), and the resulting solution was added slowly to the previously described mixture. 2-Fluoropyridine (0.138 mL, 1.606 mmol) and trimethyl(trifluoromethyl)silane (0.238 mL, 1.606 mmol) were slowly added to the reaction mixture via syringe. The resulting mixture was stirred at room temperature overnight. The reaction mixture was filtered through a pad of diatomaceous earth, and washed with ethyl acetate (100 mL). The filtrate was dried over MgSO₄, filtered and concentrated in vacuo. The crude product was purified by column chromatography on silica gel eluting with a solvent gradient of 0-10% methanol in dichloromethane to afford the title compound (55 mg, 0.099 mmol, 18.47% yield). ¹H NMR (500 MHz, DMSO-d₆) δ ppm 7.76 (s, 1H), 4.89 (p, 0.1=7.5 Hz, 1H), 3.36-3.24 (m, 1H), 2.81-2.71 (m, 2H), 2.46-2.33 (m, 8H), 1.39 (s, 9H).

### Example 119F: 3-(3-(cis-3-(trifluoromethoxy)cyclobutyl)-1,2,4-oxadiazol-5-yl)bicyclo[1.1.1]pentan-1-amine

The product from Example 119E (51 mg, 0.131 mmol) was dissolved in dichloromethane (1 mL) at 0° C. under a nitrogen atmosphere. Trifluoroacetic acid (0.124 mL, 1.611 mmol) was slowly added, and the reaction mixture was stirred at room temperature for 16 hours. The mixture was concentrated in vacuo, and the residue was taken up in methanol (3 mL) and adsorbed onto SCX (0.5 g). An SCX cartridge (3 g) was made and the pre-adsorbed suspension was added on top of the cartridge. The SCX pad was washed with methanol (60 mL), and the product was eluted with 0.7 M NH₃ in methanol (60 mL). The filtrate was concentrated in vacuo to give the title compound (43 mg, 0.107 mmol, 82% yield).

### Example 119G: (2S,4S)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide

The product from Example 119F (155 mg, 0.385 mmol) was dissolved in anhydrous dichloromethane (3 mL), the product from Example 73A (80 mg, 0.350 mmol) and N,N-diisopropylethylamine (0.244 mL, 1.400 mmol) were added, and the resulting suspension was cooled in an ice bath. A 50% solution of propanephosphonic acid anhydride (T3P) in N,N-dimethylformamide (0.409 mL, 0.700 mmol) was added, and the resulting yellow solution was stirred at 0° C. for 30 minutes, and then at room temperature for 16 hours. The reaction mixture was diluted with dichloromethane (10 mL) and washed with 1 M HCl (10 mL). The aqueous phase was extracted with dichloromethane (10 mL×2), and the organic extracts were combined, dried (MgSO₄), filtered and concentrated under reduced pressure. The crude product was purified by chromatography on silica gel using a solvent gradient of 0-100% ethyl acetate in isohexane to afford the title compound (63 mg, 0.117 mmol, 33.5% yield). ¹H NMR (500 MHz, DMSO-d₆) δ ppm 8.92 (s, 1H), 7.39 (d, J=2.7 Hz, 1H), 7.22 (dd, J=8.7, 2.7 Hz, 1H), 6.89 (d, J=8.7 Hz, 1H), 5.78 (br, 1H), 4.91 (p, J=7.5 Hz, 1H), 4.82 (dd, J=10.6, 5.8 Hz, 1H), 4.64 (dd, J=12.0, 2.3 Hz, 1H), 3.29 (s, 1H), 2.84-2.74 (m, 2H), 2.53 (d, J=10.0 Hz, 6H), 2.46-2.35 (m, 3H), 1.72 (q, J=11.8 Hz, 1H); MS (ESI) m/z 500 (M+H)⁺.

### Example 120: (2R,4R)-6-chloro-4-hydroxy-N-[(1RS,2SR,4RS,5SR)-5-{[cis-3-(trifluoromethoxy)cyclobutyl]carbamoyl}-7-oxabicyclo[2.2.1]heptan-2-yl]-3,4-dihydro-2H-1-benzopyran-2-carboxamide (Compound 219)

### Example 120A: rac-(1S,2R,4S,5R)-5-amino-N-((cis)-3-(trifluoromethoxy)cyclobutyl)-7-oxabicyclo[2.2.1]heptane-2-carboxamide Trifluoroacetic Acid

The title compound was synthesized using the same procedures as described in Example 106B through Example 106C substituting Example 86D with Example 64C. MS (APCI⁺) m/z 294.99 (M+H)⁺.

### Example 120B: (2R,4R)-6-chloro-4-hydroxy-N-[(1RS,2SR,4RS,5SR)-5-{[cis-3-(trifluoromethoxy)cyclobutyl]carbamoyl}-7-oxabicyclo[2.2.1]heptan-2-yl]-3,4-dihydro-2H-1-benzopyran-2-carboxamide

The title compound was synthesized using the same procedures as described in Example 87A through Example 87B substituting Example 86G with Example 120A and Example 10A with Example 1B. ¹H NMR (600 MHz, DMSO-d₆) δ ppm 8.09 (dd, J=7.8, 2.0 Hz, 1H), 7.93 (dd, J=10.1, 6.8 Hz, 1H), 7.38 (d, J=2.7 Hz, 1H), 7.19 (dt, J=8.7, 2.5 Hz, 1H), 6.88 (d, J=8.7 Hz, 1H), 5.68 (s, 1H), 4.79 (dd, J=10.5, 6.0 Hz, 1H), 4.68-4.58 (m, 2H), 4.56 (q, J=7.2 Hz, 1H), 4.30 (t, J=5.9 Hz, 1H), 3.96-3.84 (m, 1H), 3.84 (dd, J=7.5, 3.3 Hz, 1H), 2.75-2.62 (m, 2H), 2.41 (dd, J=9.0, 4.6 Hz, 1H), 2.31 (ddd, J=13.2, 5.9, 2.4 Hz, 1H), 2.20-2.05 (m, 2H), 1.99-1.86 (m, 2H), 1.82-1.68 (m, 1H), 1.62 (dd, J=7.9, 3.6 Hz, 1H), 1.61-1.53 (m, 1H); MS (APCI⁺) m/z 505.05 (M+H)⁺.

### Example 121: (2R,4R)-6-chloro-4-hydroxy-N-[(2S)-2-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 220)

The reaction and purification conditions described in Example 6C substituting the product of Example 124C for the product of Example 6B gave the title compound. ¹H NMR (600 MHz, DMSO-d₆) δ ppm 7.37 (dd, J=2.7, 0.9 Hz, 1H), 7.23-7.17 (m, 2H), 7.04 (s, 1H), 6.85 (d, J=8.7 Hz, 1H), 5.70 (d, J=6.3 Hz, 1H), 5.13 (d, J=4.7 Hz, 1H), 4.78 (dt, J=11.3, 6.0 Hz, 1H), 4.60 (dd, J=11.5, 2.4 Hz, 1H), 4.47 (p, J=7.1 Hz, 1H), 4.05-3.99 (m, 1H), 3.72-3.65 (m, 1H), 3.68 (s, 2H), 2.76-2.69 (m, 2H), 2.35 (ddd, J=13.1, 5.9, 2.5 Hz, 1H), 2.28 (ddd, J=12.5, 9.3, 2.8 Hz, 1H), 2.21-2.15 (m, 1H), 2.15-2.08 (m, 2H), 1.97-1.75 (m, 8H), 1.75-1.67 (m, 1H); MS (APCI⁺) m/z 563 (M+H)⁺.

### Example 122: (2R)-6-chloro-4-oxo-N-(4-{[cis-3-(trifluoromethoxy)cyclobutyl]carbamoyl}bicyclo[2.2.2]octan-1-yl)-3,4-dihydro-2H-1-benzopyran-2-carboxamide (Compound 221)

### Example 122A: Tert-Butyl (4-{[cis-3-(trifluoromethoxy)cyclobutyl]carbamoyl}bicyclo[2.2.2]octan-1-yl)carbamate

The reaction and purification conditions described in Example 2B substituting the product of Example 106A for the product of Example 2A, and 4-((tert-butoxycarbonyl)amino)bicyclo[2.2.2]octane-1-carboxylic acid (Ark Pharm) for the product of Example 1B gave the title compound. MS (APCI⁺) m/z 407 (M+H)⁺.

### Example 122B: (2R)-6-chloro-4-oxo-N-(4-{[cis-3-(trifluoromethoxy)cyclobutyl]carbamoyl}bicyclo[2.2.2]octan-1-yl)-3,4-dihydro-2H-1-benzopyran-2-carboxamide

The reaction and purification conditions described in Example 1C substituting the product of Example 122A for the product of Example 1A gave the title compound. ¹H NMR (500 MHz, DMSO-d₆) δ ppm 7.69 (s, 1H), 7.65-7.58 (m, 3H), 7.15 (dd, J=8.7, 0.6 Hz, 1H), 5.05 (dd, J=8.4, 4.9 Hz, 1H), 4.54 (p, J=7.4 Hz, 1H), 3.94-3.83 (m, 1H), 2.99-2.92 (m, 1H), 2.92-2.84 (m, 1H), 2.65-2.56 (m, 2H), 2.23-2.14 (m, 2H), 1.81-1.65 (m, 12H); MS (APCI⁺) m/z 515 (M+H)⁺.

### Example 123: (2R,4R)-6-chloro-4-hydroxy-N-(4-{[cis-3-(trifluoromethoxy)cyclobutyl]carbamoyl}bicyclo[2.2.2]octan-1-yl)-3,4-dihydro-2H-1-benzopyran-2-carboxamide (Compound 222)

The reaction and purification conditions described in Example 6C substituting the product of Example 122B for the product of Example 6B gave the title compound. ¹H NMR (500 MHz, DMSO-d₆) δ ppm 7.63 (d, J=7.9 Hz, 1H), 7.37 (dd, J=2.7, 1.0 Hz, 1H), 7.31 (s, 1H), 7.18 (ddd, J=8.7, 2.8, 0.7 Hz, 1H), 6.86 (d, J=8.7 Hz, 1H), 5.68 (s, 1H), 4.77 (dd, J=10.6, 5.9 Hz, 1H), 4.59-4.50 (m, 2H), 3.95-3.84 (m, 1H), 3.48-3.21 (m, 1H), 2.66-2.57 (m, 2H), 2.27 (ddd, J=13.0, 5.9, 2.3 Hz, 1H), 2.24-2.15 (m, 2H), 1.88-1.67 (m, 12H); MS (APCI⁺) m/z 517 (M+H)⁺.

### Example 124: (2R)-6-chloro-N-[(2S)-2-hydroxy-4-(2-{[cis-3-(trifluoromethoxy)cyclobutyl]oxy}acetamido)bicyclo[2.2.2]octan-1-yl]-4-oxo-3,4-dihydro-2H-1-benzopyran-2-carboxamide (Compound 223)

### Example 124A: Tert-Butyl [(2S)-2-hydroxy-4-(2-{[cis-3-(trifluoromethoxy)cyclobutyl]oxy}acetamido)bicyclo[2.2.2]octan-1-yl]carbamate

The reaction and purification conditions described in Example 2B substituting the product of Example 13H for the product of Example 2A, and the product of Example 13P for the product of Example 1B gave the title compound. MS (APCI⁺) m/z 453 (M+H)⁺.

### Example 124B: N-[(3S)-4-amino-3-hydroxybicyclo[2.2.2]octan-1-yl]-2-{[(1s,3R)-3-(trifluoromethoxy)cyclobutyl]oxy}acetamide

Trifluoroacetic acid (1 mL) was added to the product of Example 124A (41 mg, 0.091 mmol) and stirred at ambient temperature for 20 minutes. The mixture was concentrated under reduced pressure to give the title compound (72 mg, 0.089 mmol, 98% yield) as a trifluoroacetic acid salt with excipient trifluoroacetic acid (3 equivalents). MS (APCI⁺) m/z 453 (M+H)⁺.

### Example 124C: (2R)-6-chloro-N-(2S)-2-hydroxy-4-(2-{[cis-3-(trifluoromethoxy)cyclobutyl]oxy}acetamido)bicyclo[2.2.2]octan-1-yl-4-oxo-3,4-dihydro-2H-1-benzopyran-2-carboxamide

The reaction and purification conditions described in Example 2B substituting the product of Example 124B for the product of Example 2A gave the title compound. ¹H NMR (400 MHz, CDCl₃) δ ppm 7.88 (d, J=2.6 Hz, 1H), 7.48 (dd, J=8.8, 2.7 Hz, 1H), 7.03 (d, J=8.9 Hz, 1H), 6.53 (s, 1H), 6.19 (s, 1H), 4.84 (dd, J=12.9, 3.4 Hz, 1K), 4.35 (s, 1H), 4.31 (p, J=7.3 Hz, 1H), 4.19 (d, J=8.9 Hz, 1H), 3.74 (s, 2H), 3.68 (p, J=6.9 Hz, 1H), 3.17 (dd, J=17.3, 3.4 Hz, 1H), 2.92-2.75 (m, 3H), 2.55 (ddd, J=13.5, 8.9, 3.0 Hz, 1H), 2.41 (m, J=11.8 Hz, 1H), 2.30-2.19 (m, 2H), 2.15-2.03 (m, 3H), 2.02-1.90 (m, 3H), 1.83 (dt, J=13.3, 2.5 Hz, 1H), 1.73 (td, J=11.7, 6.0 Hz, 1H); MS (APCI⁺) m/z 561 (M+H)⁺.

### Example 125: (2R)-6-chloro-N-{3-[3-(4-chlorophenyl)-2-oxopyrrolidin-1-yl]bicyclo[1.1.1]pentan-1-yl}-4-oxo-3,4-dihydro-2H-1-benzopyran-2-carboxamide (Compound 224)

### Example 125A: Ethyl 3-(3-(4-chlorophenyl)-2-oxopyrrolidin-1-yl)bicyclo[1.1.1]pentane-1-carboxylate

A 30 mL vial was charged with iodomesitylene diacetate (289 mg, 0.79 mmol), 3-(ethoxycarbonyl)bicyclo[1.1.1]pentane-1-carboxylic acid (292 mg, 1.59 mmol, Combi-Blocks) and toluene (5 mL). The mixture was stirred at 55° C. for 30 minutes. Toluene was then removed under high vacuum. Iridium(III) bis[2-(2,4-difluorophenyl)-5-methylpyridine-N,C₂₀]-4,40-di-tert-butyl-2,20-bipyridine hexafluorophosphate (24 mg, 0.024 mmol), copper(I) thiophene-2-carboxylate (54 mg, 0.28 mmol), 4,7-diphenyl-1,10-phenanthroline (141 mg, 0.42 mmol), 2-tert-butyl-1,1,3,3-tetramethylguanidine (BTMG, 0.50 mL, 2.47 mmol) and 3-(4-chlorophenyl)pyrrolidin-2-one (230 mg, 1.18 mmol, ChemSpace) were added sequentially followed by dioxane (5.0 mL). The vial was degassed by sparging with nitrogen for 3 minutes before sealing with a polytetrafluoroethylene-lined cap. The reaction was stirred and irradiated using 2 lamps: a 40W Kessil PR160 390 nm Photoredox lamp, and an 18W 450 nm HepatoChem blue LED photoredox lamp, with forced air cooling via an electric fan blowing directly at the vial. After 18 hours, the reaction mixture was quenched by exposing to air and partitioned between water (50 mL) and dichloromethane (2×50 mL). The organic layers were combined and dried over sodium sulfate and concentrated under reduced pressure. The residue was taken up in methanol (5 mL), filtered through a glass microfiber frit and purified by reversed-phase flash chromatography [Interchim® PuriFlash® C18XS 15 μm 120 g column, flow rate 60 mL/minute, 5-100/gradient of acetonitrile in buffer (0.025 M aqueous ammonium bicarbonate, adjusted to pH 10 with ammonium hydroxide)] to give the title compound (40 mg, 0.12 mmol, 10% yield). MS (APCI⁺) m/z 334 (M+H)⁺.

### Example 125B: 3-(3-(4-chlorophenyl)-2-oxopyrrolidin-1-yl)bicyclo[1.1.1]pentane-1-carboxylic Acid

The reaction and purification conditions described in Example 110B substituting the product of Example 125A for the product of Example 110A, and ethanol for methanol gave the title compound. ¹H NMR (500 MHz, DMSO-d₆) δ ppm 12.52 (br s, 1H), 7.41-7.35 (m, 2H), 7.28-7.22 (m, 2H), 3.68 (t, J=9.2 Hz, 1H), 3.35 (d, J=7.6 Hz, 2H), 2.45-2.35 (m, 1H), 2.30-2.26 (m, 6H), 1.99 (ddt, J=12.6, 9.9, 8.5 Hz, 1H); MS (APCI⁺) m/z 306 (M+H)⁺.

### Example 125C: 2-(trimethylsilyl)ethyl (3-(3-(4-chlorophenyl)-2-oxopyrrolidin-1-yl)bicyclo[1.1.1]pentan-1-yl)carbamate

The product of Example 125B (37 mg, 0.12 mmol) was azeotroped with dry toluene 3 times. Diisopropylethylamine (0.095 mL, 0.55 mmol), 2-(trimethylsilyl)ethanol (0.35 mL, 2.42 mmol), toluene (5 mL) and diphenylphosphoryl azide (0.039 mL, 0.18 mmol) were added sequentially. Dry nitrogen was bubbled through the reaction mixture for two to three minutes. The reaction mixture was then stirred at 60° C. for 10 hours, cooled to ambient temperature, and concentrated under reduced pressure. The resulting mixture was taken up in N,N-dimethylformamide (3 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (26 mg, 0.062 mmol, 51% yield). MS (APCI⁺) m/z 421 (M+H)⁺.

### Example 125D: 1-(3-aminobicyclo[1.1.1]pentan-1-yl)-3-(4-chlorophenyl)pyrrolidin-2-one

The product of Example 125C (26 mg, 0.062 mmol) was dissolved in dichloromethane (0.5 mL) and stirred at ambient temperature. Trifluoroacetic acid (0.5 mL) was added. After stirring for 20 minutes, the reaction mixture was concentrated under reduced pressure, taken up in N,N-dimethylformamide (1 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (16 mg, 0.058 mmol, 94% yield). MS (APCI⁺) m/z 277 (M+H)⁺.

### Example 125E: (2R)-6-chloro-N-{3-[3-(4-chlorophenyl)-2-oxopyrrolidin-1-yl]bicyclo[1.1.1]pentan-1-yl}-4-oxo-3,4-dihydro-2H-1-benzopyran-2-carboxamide

The reaction and purification conditions described in Example 2B substituting the product of Example 125D for the product of Example 2A gave the title compound. ¹H NMR (400 MHz, CDCl₃) δ ppm 7.88 (d, J=2.7 Hz, 1H), 7.48 (dd, J=8.8, 2.7 Hz, 1H), 7.35-7.27 (m, 2H), 7.24-7.16 (m, 2H), 7.05 (d, J=8.8 Hz, 1H), 7.02 (s, 1H), 4.85 (dd, J=13.5, 3.3 Hz, 1H), 3.63 (t, J=9.2 Hz, 1H), 3.51-3.37 (m, 2H), 3.18 (dd, J=17.3, 3.3 Hz, 1H),2.86 (dd, J=17.3, 13.5 Hz, 1H), 2.55 (s, 6H), 2.54-2.44 (m, 1H), 2.21-2.07 (m, 1H); MS (APCI⁺) m/z 485 (M+H)⁺.

### Example 126: (2R,4R)-6-chloro-N-{3-[(3R*)-3-(4-chlorophenyl)-2-oxopyrrolidin-1-yl]bicyclo[1.1.1]pentan-1-yl}-4-hydroxy-3,4-dihydro-2H-1-benzopyran-2-carboxamide (Compound 225)

The product of Example 125E was purified by preparative chiral HPLC [CHIRALCEL® OZ-H 5 μm column, 20×250 mm, flow rate 20 mL/minute, 60% ethanol in heptane (isocratic gradient)] to give the title compound as the earlier eluting fraction wherein the stereochemistry on the lactam ring is arbitrarily assigned. ¹H NMR (90° C., 400 MHz, DMSO-d₆) δ ppm 8.36 (s, 1H), 7.40 (dd, J=2.7, 1.0 Hz, 1H), 7.37-7.32 (m, 2H), 7.30-7.22 (m, 2H), 7.16 (dd, J=8.6, 2.7 Hz, 1H), 6.88 (d, J=8.7 Hz, 1H), 5.41 (br s, 1H), 4.81 (dd, J=10.5, 5.9 Hz, 1H), 4.58 (dd, J=11.7, 2.6 Hz, 1H), 3.65 (t, J=9.0 Hz, 1H), 3.50-3.31 (m, 2H), 2.50-2.36 (m, 2H), 2.37 (s, 6H), 2.09-1.95 (m, 1H), 1.78 (ddd, J=13.0, 11.7, 10.4 Hz, 1H); MS (APCI⁺) m/z 487 (M+H)⁺.

### Example 127: (2R,4R)-6-chloro-N-{3-[(3S*)-3-(4-chlorophenyl)-2-oxopyrrolidin-1-yl]bicyclo[1.1.1]pentan-1-yl}-4-hydroxy-3,4-dihydro-2H-1-benzopyran-2-carboxamide (Compound 226)

The product of Example 125E was purified by preparative chiral HPLC [CHIRALCEL® OZ-H 5 μm column, 20×250 mm, flow rate 20 mL/minute, 60% ethanol in heptane (isocratic gradient)] to give the title compound as the later eluting fraction wherein the stereochemistry on the lactam ring is arbitrarily assigned. ¹H NMR (90° C., 400 MHz, DMSO-d₆) δ ppm 8.37 (s, 1H), 7.41-7.39 (m, 1H), 7.38-7.33 (m, 2H), 7.30-7.22 (m, 2H), 7.16 (dd, J=8.7, 2.7 Hz, 1H), 6.88 (d, J=8.7 Hz, 1H), 4.81 (dd, J=10.4, 5.8 Hz, 1H), 4.59 (dd, J=11.6, 2.6 Hz, 1H), 3.66 (t, J=9.0 Hz, 1H), 3.51-3.35 (m, 2H), 2.48-2.32 (m, 2H), 2.37 (s, 6H), 2.09-1.95 (m, 1H), 1.85-1.71 (m, 1H) MS (APCI⁺) m/z 487 (M+H)⁺.

### Example 128: (2R,4R)-6-chloro-4-hydroxy-N-(3-{5-[cis-3-hydroxycyclobutyl]-4,5-dihydro-1,2-oxazol-3-yl}bicyclo[1.1.1]pentan-1-yl)-3,4-dihydro-2H-1-benzopyran-2-carboxamide (Compound 227)

### Example 128A: Tert-Butyl (3-(hydroxymethyl)bicyclo[1.1.1]pentan-1-yl)carbamate

To a solution of 3-((tert-butoxycarbonyl)amino)bicyclo[1.1.1]pentane-1-carboxylic acid (1.00 g, 4.43 mmol) in anhydrous tetrahydrofuran (25 mL) at 0° C. under a nitrogen atmosphere was added a 1.0 M solution of borane tetrahydrofuran complex in tetrahydrofuran (8.85 mL, 8.85 mmol) dropwise, and the reaction mixture was stirred at 0° C. for 1 hour and then at room temperature for 16 hours. The reaction mixture was quenched by the careful addition of methanol (50 mL) and stirred for 10 minutes before being concentrated in vacuo. The residue was partitioned between saturated aqueous NaHCO₃ (40 mL) and ethyl acetate (75 mL×3), and the combined organic extract was dried over MgSO₄, filtered and concentrated in vacuo. The residue was purified by column chromatography on silica gel using a solvent gradient of 0-10% methanol in dichloromethane to give the title compound (0.64 g, 2.79 mmol, 63% yield). ¹H NMR (500 MHz, DMSO-d₆) δ ppm 7.40 (br. s, 1H), 4.45 (t, J=5.5 Hz, 1H), 3.42 (d, J=5.5 Hz, 2H), 1.73 (s, 6H), 1.37 (s, 9H).

### Example 128B: Tert-Butyl (3-formylbicyclo[1.1.1]pentan-1-yl)carbamate

A solution of oxalyl chloride (0.544 mL, 6.22 mmol) in anhydrous dichloromethane (12 mL) was cooled to −78° C. under a nitrogen atmosphere. A solution of dimethyl sulfoxide (0.882 mL, 12.43 mmol) in anhydrous dichloromethane (2.5 mL) was added slowly, and the reaction mixture was stirred at −78° C. for 30 minutes. A solution of the product from Example 128A (1.02 g, 4.78 mmol) in anhydrous dichloromethane (20 mL) was slowly added, and the reaction mixture was stirred at −78° C. for 30 minutes. Triethylamine (4.00 mL, 28.7 mmol) was added slowly and the reaction mixture was stirred at −78° C. for 30 minutes. The dry ice bath was removed, and the reaction mixture was allowed to warm to room temperature and stirred for 1 hour. The reaction mixture was diluted with dichloromethane (50 mL) and quenched with water (40 mL). The phases were stirred for 5 minutes. The phases were separated, and the aqueous phase was extracted with dichloromethane (75 mL×2). The organic phases were combined, dried via hydrophobic frit and concentrated in vacuo to give the title compound (1.04 g, 4.48 mmol, 94% yield). ¹H NMR (500 MHz, DMSO-d₆) δ ppm 9.59 (s, 1H), 7.65 (br. s, 1H₁),2.12 (s, 6H), 1.38 (s, 9H).

### Example 128C: Tert-Butyl (3-((hydroxyimino)methyl)bicyclo[1.1.1]pentan-1-yl)carbamate

To a solution of the product from Example 128B (950 mg, 4.50 mmol) in ethanol (23 mL) and water (2.56 mL) was added sodium acetate (1.50 g, 18.0 mmol) and hydroxylamine hydrochloride (1.88 g, 27.0 mmol), and the resulting mixture was stirred at 80° C. for 16 hours. The mixture was cooled to room temperature and diluted with ethyl acetate (100 mL) and extracted with water (50 mL). The aqueous phase was extracted with ethyl acetate (2×100 mL) and dichloromethane (2×50 mL), and the combined organic extract was dried via hydrophobic frit and concentrated under reduced pressure to give the title compound (1.32 g, 4.49 mmol, 100% yield).

### Example 128D: cis-benzyl 3-((tert-butyldiphenylsilyl)oxy)cyclobutanecarboxylate

A solution of benzyl 3-oxocyclobutanecarboxylate (8.8 g, 43.1 mmol) in anhydrous tetrahydrofuran (250 mL) under a nitrogen atmosphere was cooled to −78° C., and lithium tri-sec-butylhydroborate (1.0 M in tetrahydrofuran, 108 mL) was added slowly via syringe. The reaction mixture was stirred at −78° C. for 3 hours, and was then quenched with saturated NH₄Cl (300 mL). The mixture was warmed to room temperature and extracted with ethyl acetate (3×200 mL). The combined organic extract was dried over MgSO₄, filtered and concentrated in vacuo. The residue was purified by column chromatography on silica gel using a solvent gradient of 0-100% ethyl acetate in isohexanes to afford cis-benzyl 3-hydroxycyclobutanecarboxylate (3.85 g, 17.92 mmol, 41.6% yield). A portion of the cis-benzyl 3-hydroxycyclobutanecarboxylate (2.00 g, 9.70 mmol) and imidazole (1.452 g, 21.33 mmol) were dissolved in N,N-dimethylformamide (50 mL) and cooled in an ice-water bath. tert-Butyldiphenylchlorosilane (2.74 mL, 10.67 mmol) was added, and the reaction mixture was allowed to warm to room temperature and stirred for 3 days. The reaction mixture was concentrated in vacuo and partitioned between ethyl acetate (50 mL) and water (2×50 mL). The organic phase was washed with brine (50 mL) and dried over MgSO₄. The drying agent was filtered off, and the solvent was removed in vacuo to give the title compound (4.72 g, 8.49 mmol, 88% yield). ¹H NMR (500 MHz, DMSO-d₆) δ ppm 7.61-7.58 (m, 3H), 7.50-7.31 (m, 12H), 5.09 (s, 2H), 4.17 (tt, J=8.0, 6.8 Hz, 1H), 2.61 (tt, J=9.8, 7.7 Hz, 1H), 2.43-2.34 (m, 2H), 2.16 (dddd, J=11.5, 10.1, 6.7, 2.7 Hz, 2H), 0.98 (s, 9H).

### Example 128E: cis-3-((tert-butyldiphenylsilyl)oxy)-N-methoxy-N-methylcyclobutanecarboxamide

A solution of the product from Example 128D (4.70 g, 10.57 mmol) in tetrahydrofuran (30 mL) was cooled in an ice-water bath, and 1.0 M NaOH (26.4 mL, 26.43 mmol) was added slowly. The reaction mixture was stirred at 50° C. for 16 hours. The mixture was concentrated in vacuo and the basic aqueous mixture was extracted with ethyl acetate (40 mL). The organic layer was dried over MgSO₄, filtered and concentrated in vacuo to give cis-3-((tert-butyldiphenylsilyl)oxy)cyclobutanecarboxylic acid (1.54 g, 2.259 mmol, 21.37% yield) as a colorless oil. The oil (1.52 g, 4.29 mmol) was combined with N,O-dimethylhydroylamine hydrochloride (0.502 g, 5.15 mmol) in anhydrous dichloromethane (30 mL) and cooled in an ice-water bath. Hunig's base (3.00 mL, 17.15 mmol) was added, followed by 2-(3H-[1,2,3]triazolo[4,5-b]pyridin-3-yl)-1,1,3,3-tetramethylisouronium hexafluorophosphate(V) (2.445 g, 6.43 mmol), and the reaction mixture stirred at room temperature for 24 hours. The mixture was diluted with ethyl acetate (75 mL) and washed with 1 M HCl (30 mL), saturated aqueous NaHCO₃ (30 mL) and brine (40 mL×3). The organic phase was dried over MgSO₄ and concentrated in vacuo to give the title compound (1.16 g, 1.751 mmol, 40.8% yield). ¹H NMR (500 MHz, DMSO-d₆) δ ppm 7.63-7.58 (m, 4H), 7.49-7.41 (m, 6H), 4.19 (p, J=7.4 Hz, 1H), 3.58 (s, 3H), 3.07 (s, 3H), 2.84 (s, 1H), 2.28 (dtt, J=9.9, 7.1, 2.6 Hz, 2H), 2.17-2.08 (m, 2H), 0.98 (s, 9H).

### Example 128F: cis-3-((tert-Butyldiphenylsilyloxy)cyclobutanecarbaldehyde

A solution of the product from Example 128E (6.24 g, 15.69 mmol) in anhydrous tetrahydrofuran (150 mL) under a nitrogen atmosphere was cooled to −78° C., and diisobutylaluminum hydride (1.0 M in toluene) (34.5 mL, 34.5 mmol) was slowly added via syringe. The reaction mixture was stirred at −78° C. for 2 hours. Methanol (1 mL) was added and the reaction mixture was stirred at −78° C. for 10 minutes. Saturated Rochelle salt solution (150 mL) and ethyl acetate (150 mL) were added and the dry-ice bath was removed. The mixture was stirred vigorously while warming to room temperature. The phases were separated, and the aqueous phase was extracted with ethyl acetate (2×100 mL). The combined organic extract was dried over MgSO₄, filtered and concentrated under reduced pressure. The residue was purified by column chromatography on silica gel, eluting with a solvent gradient of 0-50% tert-butyl methyl ether in isohexanes to afford the title compound (4.82 g, 13.53 mmol, 86% yield). ¹H NMR (500 MHz, DMSO-d₆) δ ppm 9.66 (s, 0.15H), 9.56 (s, 0.85H), 7.65-7.55 (m, 4H), 7.52-7.40 (m, 6H), 4.30-4.20 (m, 1H), 2.70-2.57 (m, 1H), 2.34-2.22 (m, 2H), 2.19-2.08 (m, 2H), 0.99 (s, 9H).

### Example 128G: tert-butyldiphenyl(cis-3-vinylcyclobutoxy)silane

A 2.5 M solution of n-butyllithium in hexanes (2.481 mL, 6.20 mmol) was added slowly to a suspension of methyltriphenylphosphonium bromide (2.216 g, 6.20 mmol) in anhydrous tetrahydrofuran (50 mL) at room temperature under a nitrogen atmosphere. The suspension was stirred at room temperature for 1 hour and then cooled to −78° C. A solution of the product from Example 128F (2.00 g, 5.91 mmol) in anhydrous tetrahydrofuran (50 mL) was added slowly, and the reaction mixture was stirred at −78° C. for 1 hour. The mixture was allowed to warm to room temperature and stirred overnight. The mixture was concentrated in vacuo and purified by column chromatography on silica gel using a solvent gradient of 0-100% tert-butyl methyl ether in isohexane) to yield the title compound (1.23 g, 3.47 mmol, 58.8% yield). ¹H NMR (500 MHz, DMSO-d₆) δ ppm 7.62-7.58 (m, 4H), 7.46-7.41 (m, 6H), 5.95-5.73 (m, 1H), 5.09-4.70 (m, 2H), 4.14-4.04 (m, 1H), 2.33-2.17 (m, 3H), 1.85-1.67 (m, 2H), 0.97 (s, 9H).

### Example 128H: Tert-Butyl (3-{5-[cis-3-{[tert-butyl(diphenyl)silyl]oxy}cyclobutyl]-4,5-dihydro-1,2-oxazol-3-yl}bicyclo[1.1.1]pentan-1-yl)carbamate

A solution of the product from Example 128C (1.32 g, 5.83 mmol) in anhydrous N,N-dimethylformamide (12.5 mL) was cooled in an ice-water bath while a solution of N-chlorosuccinimide (0.857 g, 6.42 mmol) in anhydrous N,N-dimethylformamide (12.5 mL) was slowly added. The reaction mixture was stirred at 0° C. for 30 minutes and at room temperature for 3 hours. A solution of the product from Example 128G (1.189 g, 3.53 mmol) in anhydrous N,N-dimethylformamide (6 mL) was added, followed by triethylamine (0.739 mL, 5.30 mmol), and the reaction mixture was stirred at 60° C. for 16 hours. The mixture was diluted with ethyl acetate (100 mL) and washed with 1 M HCl (50 mL). The aqueous phase was extracted with ethyl acetate (75 mL 2), and the combined organic extract was washed with brine (3×100 mL), dried via hydrophobic frit, and concentrated in vacuo. The crude product was purified by column chromatography on silica gel using a solvent gradient of 0-50% ethyl acetate in isohexane to afford the title compound (1.27 g, 2.129 mmol, 60.2% yield). ¹H NMR (500 MHz, DMSO-d₆) δ ppm 7.63-7.57 (m, 4H), 7.49-7.40 (m, 6H), 4.48-4.41 (m, 1H), 4.10-4.04 (m, 1H), 2.93 (dd, J=17.5, 10.5 Hz, 1H), 2.42 (dd, J=17.5, 7.5 Hz, 1H), 2.18-2.01 (m, 8H), 1.83-1.73 (m, 2H), 1.73-1.62 (m, 1H), 1.38 (s, 9H), 0.98 (s, 9H).

### Example 128I: Tert-Butyl (3-{5-[cis-3-hydroxycyclobutyl]-4,5-dihydro-1,2-oxazol-3-yl}bicyclo[1.1.1]pentan-1-yl)carbamate

A solution of the product from Example 128H (1.27 g, 2.265 mmol) in anhydrous tetrahydrofuran (20 mL) was cooled in an ice-water bath, and a 1.0 M solution of tetra-N-butylammonium fluoride in tetrahydrofuran (3.40 mL, 3.40 mmol) was added. The reaction mixture was stirred at 0° C. for 90 minutes, and then was allowed to warm to room temperature and stirred for 16 hours. The mixture was concentrated in vacuo and the crude product was purified by column chromatography on silica gel using a solvent gradient of 0-100% ethyl acetate in isohexane to afford the title compound (660 mg, 1.945 mmol, 86% yield). ¹H NMR (500 MHz, DMSO-d₆) δ ppm 7.60 (br. s, 1H), 4.96 (d, J=6.5 Hz, 1H), 4.45-4.38 (m, 1H), 3.92-3.82 (m, 1H), 2.94 (dd, J=17.0, 10.5 Hz, 1H), 2.48-2.41 (m, 1H), 2.06 (s, 8H), 1.84-1.72 (m, 1H), 1.60-1.43 (m, 2H), 1.37 (s, 9H).

### Example 128J: (2R,4R)-6-chloro-4-hydroxy-N-(3-{5-[cis-3-hydroxycyclobutyl]-4,5-dihydro-1,2-oxazol-3-yl}bicyclo[1.1.1]pentan-1-yl)-3,4-dihydro-2H-1-benzopyran-2-carboxamide

The title compound was prepared using the methods described for the synthesis of Example 131D, substituting the product from Example 128I for the product from Example 131C, and substituting the product from Example 3B for the product from Example 73B. ¹H NMR (500 MHz, DMSO-d₆) δ ppm 8.74 (br. s, 1H), 7.38 (dd, J=2.5, 1.0 Hz, 1H), 7.20 (dd, J=8.5, 2.5 Hz, 1H), 6.88 (d, J=8.5 Hz, 1H), 5.75-5.61 (m, 1H), 4.97 (d, J=6.5 Hz, 1H), 4.85-4.75 (m, 1H), 4.59 (dd, J=12.0, 2.5 Hz, 1H), 4.47-4.38 (m, 1H), 3.96-3.82 (m, 1H), 2.97 (dd, J=17.0, 10.5 Hz, 1H), 2.49-2.46 (m, 1H), 2.39-2.30 (m, 1H), 2.25-2.09 (m, 8H), 1.85-1.75 (m, 1H), 1.75-1.63 (m, 1H), 1.62-1.44 (m, 2H); MS (ESI) m/z 433 (M+H)⁺.

### Example 129: (2S,4R)-6-chloro-4-hydroxy-N-(3-{5-[cis-3-hydroxycyclobutyl]-4,5-dihydro-1,2-oxazol-3-yl}bicyclo[1.1.1]pentan-1-yl)-3,4-dihydro-2H-1-benzopyran-2-carboxamide (Compound 228)

The title compound was prepared using the methods described for the synthesis of Example 131D, substituting the product from Example 128I for the product from Example 131C. ¹H NMR (500 MHz, DMSO-d₆) δ ppm 8.80 (br. s, 1H) 7.31 (d, J=2.5 Hz, 1H), 7.25 (dd, J=8.5, 2.5 Hz, 1H), 6.93 (d, J=8.5 Hz, 1H), 5.72-5.50 (m, 1H), 5.08-4.88 (m, 1H), 4.58 (t, J=3.5 Hz, 1H), 4.54 (dd, J=11.0, 2.5 Hz, 1H), 4.48-4.37 (m, 1H), 3.94-3.83 (m, 1H), 2.97 (dd, J=17.0, 10.5 Hz, 1H), 2.47-2.43 (m, 1H), 2.23-2.05 (m, 9H), 1.93-1.85 (m, 1H), 1.83-1.76 (m, 1H), 1.60-1.45 (m, 2H); MS (ESI) m/z 433 (M+H)⁺.

### Example 130: (2R,4R)-6-chloro-4-hydroxy-N-(3-{3-[cis-3-(trifluoromethoxy)cyclobutyl]-1,2,4-oxadiazol-5-yl}bicyclo[1.1.1]pentan-1-yl)-3,4-dihydro-2H-1-benzopyran-2-carboxamide (Compound 229)

The title compound was prepared using the method described for the synthesis of Example 119G, substituting the product from Example 3B for the product from Example 73A. ¹H NMR (500 MHz, DMSO-d₆) δ ppm 8.91 (s, 1H), 7.39 (d, J=2.8 Hz, 1H), 7.22 (dd, J=8.7, 2.7 Hz, 1H), 6.89 (d, J=8.7 Hz, 1H), 5.73 (br s, 1H), 4.91 (p, J=7.6 Hz, 1H), 4.82 (dd, J=10.7, 5.8 Hz, 1H), 4.64 (dd, J=12.0, 2.3 Hz, 1H), 3.30 (s, 1H), 2.83-2.74 (m, 2H), 2.54 (s, 6H), 2.47-2.34 (m, 3H), 1.76-1.67 (m, 1H); MS (ESI) m/z 498 (M−H)⁻.

### Example 131: (2S,4R)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide (Compound 230)

### Example 131A (Z)-4-chloro-3-fluoro-N′-hydroxybenzimidamide

To a solution of 4-chloro-3-fluorobenzonitrile (2.5 g, 16.07 mmol) in ethanol (20 mL) was added hydroxylamine (2.5 mL, 40.8 mmol) and the resulting solution heated at reflux for 16 hours. After this time, the reaction mixture was cooled to room temperature and the volatiles were removed under reduced pressure. The resulting solid was triturated with dichloromethane/isohexane (3:1, 50 mL) to give the title compound (2.82 g, 14.21 mmol, 87% yield). ¹H NMR (500 MHz, DMSO-d₆) δ ppm 9.87 (s, 1H), 7.65 (dd, J=11.0, 1.9 Hz, 1H), 7.62-7.53 (m, 2H), 5.95 (s, 2H).

### Example 131B (E)-tert-butyl (3-(((4-chloro-3-fluorophenyl)(hydroxyimino)methyl)carbamoyl)bicyclo[1.1.1]pentan-1-yl)carbamate

The product from Example 131A (190 mg, 1.01 mmol) and 3-((tert-butoxycarbonyl)amino)bicyclo[1.1.1]pentane-1-carboxylic acid (191 mg, 0.840 mmol) were dissolved in anhydrous N,N-dimethylformamide (11 mL) at 0° C. under a nitrogen atmosphere. N,N-Diisopropylethylamine (0.440 mL, 2.52 mmol) and (1-[bis(dimethylamino)methylene]-1H-1,2,3-triazolo[4,5-b]pyridinium 3-oxid hexafluorophosphate) (HATU, 383 mg, 1.009 mmol) were added and the reaction mixture was stirred at 0° C. for 10 minutes and then at ambient temperature for 16 hours. The reaction mixture was poured into HCl (0.5 M, 50 mL) and extracted with dichloromethane (3×50 mL). The organic extracts were combined, passed through a phase separator and concentrated under reduced pressure. The residue was purified by chromatography on silica gel using a solvent gradient of 0-10% methanol in dichloromethane to afford the title compound (295 mg, 0.704 mmol, 84% yield). ¹H NMR (500 MHz, DMSO-d₆) δ ppm 7.76-7.68 (m, 2H), 7.67 (m, 1H), 7.60 (dd, J=8.4, 2.0 Hz, 1H), 6.90 (s, 2H), 2.25 (s, 6H), 1.39 (s, 9H).

### Example 131C: Tert-Butyl (3-(3-(4-chloro-3-fluorophenyl)-1,2,4-oxadiazol-5-yl)bicyclo[1.1.1]pentan-1-yl)carbamate

The product from Example 131B (583 mg, 0.733 mmol) was dissolved in anhydrous tetrahydrofuran (10 mL) under a nitrogen atmosphere. The solution was cooled to 0° C. and tetra-n-butylammonium fluoride (1 M in tetrahydrofuran) (1.832 mL, 1.832 mmol) was added slowly via syringe. The reaction mixture was stirred at room temperature for 15 minutes and then at 60° C. for 16 hours. The reaction mixture was concentrated under reduced pressure to give a residue that was purified by chromatography on silica gel using a solvent gradient of 0-100% ethyl acetate in isohexane to afford the title compound (120 mg, 0.215 mmol, 29.3% yield).

### Example 131D: (2S,4R)-6-chloro-N-{3-[3-(4-chloro-3-fluorophenyl)-1,2,4-oxadiazol-5-yl]bicyclo[1.1.1]pentan-1-yl}-4-hydroxy-3,4-dihydro-2H-1-benzopyran-2-carboxamide

To a solution of the product from Example 131C (120 mg, 0.316 mmol) in dichloromethane (10 mL) was added trifluoroacetic acid (0.024 mL, 0.316 mmol) and the resulting mixture was stirred at room temperature for 16 hours. The solvent was removed in vacuo to give 3-(3-(4-chloro-3-fluorophenyl)-1,2,4-oxadiazol-5-yl)bicyclo[1.1.1]pentan-1-amine, trifluoroacetic acid (134 mg, 0.317 mmol, 100% yield). A portion of the 3-(3-(4-chloro-3-fluorophenyl)-1,2,4-oxadiazol-5-yl)bicyclo[1.1.1]pentan-1-amine, trifluoroacetic acid (38.7 mg, 0.098 mmol) was combined with the product from Example 73B (15 mg, 0.066 mmol) and N,N-diisopropylethylamine (0.080 mL, 0.459 mmol) in anhydrous N,N-dimethylformamide (1 mL) under a nitrogen atmosphere. The resulting mixture was cooled in an ice-water bath, and a 50% solution of propanephosphonic acid anhydride (T3P®) in N,N-dimethylformamide (0.046 mL, 0.079 mmol) was added. The resulting solution was allowed to warm to room temperature and was stirred for 3 hours. The reaction mixture was purified by preparative HPLC [Waters XBridge™ C18 5 μm OBD column, 19×50 mm, 10-40% gradient of acetonitrile in buffer (0.1% aqueous ammonium bicarbonate)] to afford the title compound (5.3 mg, 16% yield). ¹H NMR (500 MHz, DMSO-d₆) δ ppm 9.00 (s, 1H), 7.95 (dd, J=9.7, 1.9 Hz, 1H), 7.90-7.85 (m, 1H), 7.82 (dd, J=8.3, 7.4 Hz, 1H), 7.33 (d, J=2.7 Hz, 1H), 7.27 (dd, J=8.7, 2.7 Hz, 1H), 6.95 (d, J=8.8 Hz, 1H), 5.65 (s, 1H), 4.63-4.56 (m, 2H), 2.60 (s, 6H), 2.13 (dt, J=13.8, 3.4 Hz, 1H), 1.93 (ddd, J=14.1, 10.9, 3.7 Hz, 1H); MS (ESI) m/z 488 (M−H)⁻.

### Example 132: (2S,4R)-6-chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide (Compound 231)

### Example 132A: Tert-Butyl (3-(4-(6-(trifluoromethyl)pyridin-3-yl)-1H-imidazol-1-yl)bicyclo[1.1.1]pentan-1-yl)carbamate

To a solution of 6-(trifluoromethyl)nicotinaldehyde (2.5 g, 14.28 mmol) in a 2:1 mixture of ethanol and tetrahydrofuran (100 mL) was added 1-((isocyanomethyl)sulfonyl)-4-methylbenzene (3.27 g, 16.75 mmol) and sodium cyanide (0.105 g, 2.143 mmol) dissolved in a small amount of water. The mixture was stirred at room temperature for 3 hours and was concentrated under reduced pressure. Ethyl acetate (100 mL) was added, and the solution was dried over MgSO₄, filtered and concentrated in vacuo to give 4-tosyl-5-(6-(trifluoromethyl)pyridin-3-yl)-4,5-dihydrooxazole (5.25 g, 12.76 mmol, 89% yield). A portion of this solid (1.04 g, 2.81 mmol) was combined with tert-butyl (3-aminobicyclo[1.1.1]pentan-1-yl)carbamate (1.00 g, 5.04 mmol) and xylene (50 mL), and the mixture was heated at 135° C. while stirring for 16 hours. The mixture was concentrated in vacuo, and the residue was purified by chromatography on silica gel using a solvent gradient of 0-100% ethyl acetate in isohexane to afford the title compound (243 mg, 0.561 mmol, 19.97% yield). ¹H NMR (500 MHz, DMSO-d₆) δ ppm 9.17-9.12 (m, 1H), 8.35 (dd, J=8.1, 2.1 Hz, 1H), 8.11-8.06 (m, 1H), 7.92-7.84 (m, 2H), 7.75 (s, 1H), 2.43 (s, 6H), 1.41 (s, 9H).

### Example 132B: (2S,4R)-6-Chloro-4-hydroxy-N-(3-{4-[6-(trifluoromethyl)pyridin-3-yl]-1H-imidazol-1-yl}bicyclo[1.1.1]pentan-1-yl)-3,4-dihydro-2H-1-benzopyran-2-carboxamide

The title compound was prepared using the procedures described for the synthesis of Example 131D, substituting the product from Example 132A for the product from Example 131C. ¹H NMR (500 MHz, DMSO-d₆) δ ppm 9.14 (d, J=2.1 Hz, 1H), 8.99 (s, 1H), 8.36 (dd, J=8.2, 2.2 Hz, 1H), 8.13 (d, J=1.3 Hz, 1H), 7.93 (d, J=1.2 Hz, 1H), 7.90 (d, J=8.1 Hz, 1H), 7.34 (d, J=2.7 Hz, 1H), 7.28 (dd, J=8.7, 2.7 Hz, 1H), 6.96 (d, J=8.7 Hz, 1H), 5.65 (d, J=4.7 Hz, 1H), 4.64-4.58 (m, 2H), 2.58 (s, 6H), 2.14 (dt, J=13.9, 3.3 Hz, 1H), 1.94 (ddd, J=14.2, 11.0, 3.6 Hz, 1H); MS (ESI) m/z 505 (M+H)⁺.

### Example 133: (2R,4R)-6-chloro-4-hydroxy-N-{3-(5-[cis-3-(trifluoromethoxy)cyclobutyl]-4,5-dihydro-1,2-oxazol-3-yl}bicyclo[1.1.1]pentan-1-yl)-3,4-dihydro-2H-1-benzopyran-2-carboxamide (Compound 232)

The title compound was prepared using the methods described for the synthesis of Example 131D, substituting the product from Example 134A for the product from Example 131C, and substituting the product from Example 3B for the product from Example 73B. ¹H NMR (500 MHz, CDCl₃) δ ppm 7.45 (d, J=2.5 Hz, 1H), 7.18 (dd, J=8.5, 2.5 Hz, 1H), 6.97 (s, 1H), 6.84 (d, J=8.5 Hz, 1H), 4.96-4.89 (m, 1H), 4.62-4.55 (m, 2H), 4.55-4.47 (m, 1H), 2.96 (dd, J=17.0, 10.5 Hz, 1H), 2.66 (ddd, J=13.5, 5.5, 3.0 Hz, 1H), 2.51-2.31 (m, 9H), 2.19-2.00 (m, 4H); MS (ESI) m/z 501 (M+H)⁺.

### Example 134: (2S,4R)-6-chloro-4-hydroxy-N-(3-{5-[cis-3-(trifluoromethoxy)cyclobutyl]-4,5-dihydro-1,2-oxazol-3-yl}bicyclo[1.1.1]pentan-1-yl)-3,4-dihydro-2H-1-benzopyran-2-carboxamide (Compound 233)

### Example 134A: Tert-Butyl (3-{5-[cis-3-(trifluoromethoxy)cyclobutyl]-4,5-dihydro-1,2-oxazol-3-yl}bicyclo[1.1.1]pentan-1-yl)carbamate

The title compound was prepared using the method described for the synthesis of Example 119e, substituting the product from Example 128I for the product from Example 119D. ¹H NMR (500 MHz, DMSO-d₆) δ ppm 7.61 (br. s, 1H), 4.71-4.63 (m, 1H), 4.54-4.48 (m, 1H), 2.99 (dd, J=17.5, 10.5 Hz, 1H), 2.49-2.44 (m, 1H), 2.38-2.30 (m, 2H), 2.09-1.94 (m, 8H), 1.90-1.80 (m, 1H), 1.37 (s, 9H).

### Example 134B: (2S,4R)-6-chloro-4-hydroxy-N-(3-{S-[cis-3-(trifluoromethoxy)cyclobutyl]-4,5-dihydro-1,2-oxazol-3-yl}bicyclo[1.1.1]pentan-1-yl)-3,4-dihydro-2H-1-benzopyran-2-carboxamide

The title compound was prepared using the methods described for the synthesis of Example 131D, substituting the product from Example 134A for the product from Example 131C. ¹H NMR (500 MHz, CDCl₃) δ ppm 7.32 (d, J=2.5 Hz, 1H), 7.22 (dd, J=9.0, 2.5 Hz, 1H), 7.04 (s, 1H), 6.90 (d, J=9.0 Hz, 1H), 4.83-4.78 (m, 1H), 4.68 (dd, J=12.0, 2.5 Hz, 1H), 4.63-4.56 (m, 1H), 4.56-4.46 (m, 1H), 2.98 (dd, J=17.0, 10.5 Hz, 1H), 2.57-2.33 (m, 10H), 2.18-2.03 (m, 3H), 2.03-1.92 (m, 2H), MS (ESI) m/z 501 (M+H)⁺.

### Example 135: (2R,4R)-6-chloro-4-hydroxy-N-{3-(5-[cis-3-(trifluoromethoxy)cyclobutyl]-1,2-oxazol-3-yl}bicyclo[1.1.1]pentan-1-yl)-3,4-dihydro-2H-1-benzopyran-2-carboxamide (Compound 234)

### Example 135A: tert-butyl(cis-3-ethynylcyclobutoxy)diphenylsilane

To a solution of the product from Example 128F (2.00 g, 5.91 mmol) in methanol (50 mL) was added K₂CO₃ (1.960 g, 14.18 mmol), and the resulting mixture was stirred at room temperature for 10 minutes. Dimethyl (1-diazo-2-oxopropyl)phosphonate (1.703 mL, 7.09 mmol) was added slowly via syringe, and the reaction mixture was stirred at room temperature for 3 hours. The reaction mixture was concentrated onto silica gel, and the crude product was purified by column chromatography on silica gel, eluting with a solvent gradient of 0-50% tert-butyl methyl ether in isohexanes to afford the title compound (1.54 g, 4.14 mmol, 70.1% yield). ¹H NMR (500 MHz, DMSO-d₆) δ ppm 7.62-7.57 (m, 4H), 7.50-7.41 (m, 6H), 4.12-4.08 (m, 1H), 2.95 (d, J=2.2 Hz, 1H), 2.49-2.27 (m, 3H), 2.03-1.97 (m, 2H), 0.98 (s, 9H).

### Example 135B: Tert-Butyl (3-(5-(cis-3-((tert-butyldiphenylsilyl)oxy)cyclobutyl)isoxazol-3-yl)bicyclo[1.1.1]pentan-1-yl)carbamate

The title compound was prepared using the methods described for the synthesis of Example 128H, substituting the product from Example 135A for the product from Example 128G. ¹H NMR (500 MHz, DMSO-d₆) δ ppm 7.65 (br s, 1H), 7.64-7.58 (m, 4H), 7.46 (dddd, J=14.1, 8.6, 5.7, 2.5 Hz, 6H), 6.25 (s, 1H), 4.25 (p, J=7.2 Hz, 1H), 3.00 (ddd, J=17.7, 10.1, 7.6 Hz, 1H), 2.57-2.52 (m, 2H), 2.19 (s, 6H), 2.16-2.12 (m, 2H), 1.39 (s, 9H), 0.99 (s, 9H).

### Example 135C: Tert-Butyl (3-(5-(cis-3-(trifluoromethoxy)cyclobutyl)isoxazol-3-yl)bicyclo[1.1.1]pentan-1-yl)carbamate

To a stirred solution of the product from Example 135B (402 mg, 0.719 mmol) in tetrahydrofuran (7.5 mL) was added a 1.0 M solution of tetra-N-butylammonium fluoride in tetrahydrofuran (1.08 mL, 1.08 mmol), and the resulting solution was stirred at room temperature for 48 hours. The reaction mixture was absorbed onto silica and the crude product was purified by column chromatography on silica gel using a solvent gradient of 0-100% ethyl acetate in isohexane to afford tert-butyl (3-(5-(cis-3-hydroxycyclobutyl)isoxazol-3-yl)bicyclo[1.1.1]pentan-1-yl)carbamate (148 mg, 61% yield). A mixture of silver(I) trifluoromethanesulfonate (356 mg, 1.386 mmol), potassium fluoride (107 mg, 1.848 mmol) and 1-chloromethyl-4-fluoro-1,4-diazoniabicyclo[2.2.2]octane bis(tetrafluoroborate) (245 mg, 0.693 mmol, Selectfluor™) was stirred under a nitrogen atmosphere in a flask wrapped with aluminum foil. The flask was cooled in a water bath, and a solution of tert-butyl (3-(5-(cis-3-hydroxycyclobutyl)isoxazol-3-yl)bicyclo[1.1.1]pentan-1-yl)carbamate (148 mg, 0.462 mmol) in 4:1 ethyl acetate:tetrahydrofuran (10 mL) was added slowly to the reaction mixture. 2-Fluoropyridine (0.12 mL, 1.39 mmol) and trimethyl(trifluoromethyl)silane (0.205 mL, 1.386 mmol) were slowly added to the reaction mixture via syringe. The reaction mixture was stirred at room temperature for 3 days. The mixture was adsorbed onto silica and the crude product was purified by column chromatography on silica gel, eluting with a solvent gradient of 0-100% ethyl acetate in isohexane to afford the title compound (82 mg, 37%). ¹H NMR (500 MHz, DMSO-d₆) δ ppm 7.65 (s, 1H), 6.38 (s, 1H), 4.85 (p, =7.4 Hz, 1H), 2.82-2.72 (m, 2H), 2.41-2.29 (m, 3H), 2.23-2.11 (m, 6H), 1.38 (s, 9H).

### Example 135D: (2R,4R)-6-chloro-4-hydroxy-N-(3-{5-[cis-3-(trifluoromethoxy)cyclobutyl]-1,2-oxazol-3-yl}bicyclo[1.1.1]pentan-1-yl)-3,4-dihydro-2H-1-benzopyran-2-carboxamide

The title compound was prepared using the methods described for the synthesis of Example 131D, substituting the product from Example 135C for the product from Example 131C, and substituting the product from Example 3B for the product from Example 73B. ¹H NMR (500 MHz, DMSO-d₆) δ ppm 8.79 (s, 1H), 7.38 (d, J=2.5 Hz, 1H), 7.21 (dd, J=8.5, 2.5 Hz, 1H), 6.89 (d, J=8.5 Hz, 1H), 6.43 (s, 1H), 5.71 (d, J=6.5 Hz, 1H), 4.90-4.77 (m, 2H), 4.61 (dd, J=12.0, 2.0 Hz, 1H), 2.83-2.74 (m, 2H), 2.41-2.29 (m, 10H), 1.76-1.65 (m, 1H); MS (ESI) m/z 497 (M−H)⁻.

### Example 136: (2R,4R)-6-chloro-N-[3-(5-chloro-1H-indazol-1-yl)bicyclo[1.1.1]pentan-1-yl]-4-hydroxy-3,4-dihydro-2H-1-benzopyran-2-carboxamide (Compound 235)

### Example 136A: Methyl 3-(5-chloro-1H-indazol-1-yl)bicyclo[1.1.1]pentane-1-carboxylate

A 30 mL vial was charged with iodomesitylene diacetate (0.57 g, 1.6 mmol), 3-(methoxycarbonyl)bicyclo[1.1.1]pentane-1-carboxylic acid (0.58 g, 3.2 mmol, Synthonix) and toluene (7 mL). The mixture was stirred at 60° C. for 30 minutes. Toluene was then removed under high vacuum. Tris(2-phenylpyridine)iridium (10.3 mg, 0.016 mmol), copper(II) acetylacetonate (103 mg, 0.39 mmol), and 5-chloro-1H-indazole (0.12 g, 0.79 mmol) were added followed by dioxane (2.0 mL). The vial was degassed by sparging with nitrogen for 3 minutes before sealing with a polytetrafluoroethylene-lined cap. The reaction was stirred and irradiated using 2 lamps: a 40W Kessil PR160 390 nm Photoredox lamp, and a 18W 450 nm HepatoChem blue LED photoredox lamp. Both lamps were placed 3 cm away from the reaction vial set inside a continuously running tap water bath. The reaction temperature was measured to be 18° C. and maintained at that temperature for the duration of the reaction. After 4 hours, the reaction mixture was quenched by exposing to air and partitioned between water (100 mL) and dichloromethane (2×50 mL). The organic layers were combined and dried over sodium sulfate and concentrated under reduced pressure. The residue was taken up in methanol (10 mL), filtered through a glass microfiber frit and purified by preparative HPLC [YMC TriArt™ C18 Hybrid 5 μm column, 50×100 mm, flow rate 140 mL/minute, 20-100% gradient of acetonitrile in buffer (0.025 M aqueous ammonium bicarbonate, adjusted to pH 10 with ammonium hydroxide)] to give the title compound (189 mg, 0.68 mmol, 87% yield). MS (ESI⁺) m/z 277 (M+H)⁺.

### Example 136B: 3-(5-chloro-1H-indazol-1-yl)bicyclo[1.1.1]pentane-1-carboxylic Acid

The reaction and purification conditions described in Example 110B substituting the product of Example 136A for the product of Example 110A gave the title compound. MS (APCI⁺) m/z 263 (M+H)⁺.

### Example 136C: 3-(5-chloro-1H-indazol-1-yl)bicyclo[1.1.1]pentan-1-amine

The reaction and purification conditions described in Examples 125C and 125D substituting the product of Example 136B for the product of Example 125B gave the title compound. MS (APCI⁺) m/z 234 (M+H)⁺.

### Example 136D: (2R,4R)-6-chloro-N-[3-(5-chloro-1H-indazol-1-yl)bicyclo[1.1.1]pentan-1-yl]-4-hydroxy-3,4-dihydro-2H-1-benzopyran-2-carboxamide

The product of Example 1B (26 mg, 0.12 mmol), the product of Example 136C (27 mg, 0.12 mmol) and triethylamine (0.081 mL, 0.58 mmol) were combined with N,N-dimethylformamide (2 mL) and stirred at ambient temperature. 1-[Bis(dimethylamino)methylene]-1H-1,2,3-triazolo[4,5-b]pyridinium 3-oxid hexafluorophosphate (57 mg, 0.15 mmol, HATU) was added. The resulting suspension was stirred for 1 hour, and then partitioned between dichloromethane (2×25 mL) and aqueous sodium carbonate (1.0 M, 20 mL). The organic layers were combined and dried over sodium sulfate and concentrated under reduced pressure. The residue was taken up in methanol (2 mL). To the resulting solution stirring at ambient temperature, sodium borohydride (53 mg, 1.4 mmol) was added in one portion. After stirring for 10 minutes, saturated aqueous ammonium chloride solution (0.1 mL) was added. The resulting mixture was combined with diatomaceous earth (about 2 grams) and concentrated under reduced pressure to a free flowing powder, and the powder was directly purified by reversed-phase flash chromatography [Custom packed YMC TriArt™ C18 Hybrid 20 μm column, 25×150 mm, flow rate 70 mL/minute, 5-100% gradient of acetonitrile in buffer (0.025 M aqueous ammonium bicarbonate, adjusted to pH 10 with ammonium hydroxide)] to give the title compound (39 mg, 0.09 mmol, 76% yield). ¹H NMR (500 MHz, DMSO-d₆) δ ppm 8.97 (s, 1H), 8.10 (d, J=1.0 Hz, 1H), 7.89 (dd, J=2.0, 0.7 Hz, 1H), 7.77 (dt, J=9.1, 0.9 Hz, 1H), 7.42 (dd, J=8.9, 2.0 Hz, 1H), 7.39 (dd, J=2.7, 1.0 Hz, 1H), 7.22 (ddd, J=8.7, 2.7, 0.7 Hz, 1H), 6.91 (d, J=8.7 Hz, 1H), 5.74 (s, 1H), 4.87-4.81 (m, 1H), 4.68 (dd, J=12.0, 2.3 Hz, 1H), 2.72 (s, 6H), 2.39 (ddd, J=12.8, 5.9, 2.3 Hz, 1H), 1.74 (ddd, J=12.9, 12.1, 10.8 Hz, 1H); MS (APCI⁺) m/z 555 (M+H)⁺.

### Example 137: (2S,4R)-6-chloro-N-{3-[4-(4-chlorophenyl)-1H-pyrazol-1-yl]bicyclo[1.1.1]pentan-1-yl}-4-hydroxy-3,4-dihydro-2H-1-benzopyran-2-carboxamide (Compound 236)

The reaction and purification conditions described in Example 2B substituting the product of Example 110C for the product of Example 2A, and the product of Example 73B for the product of Example 1B gave the title compound. ¹H NMR (600 MHz, DMSO-d₆) δ ppm 8.98 (s, 1H), 8.32 (d, J=0.8 Hz, 1H), 7.97 (d, J=0.8 Hz, 1H), 7.66-7.61 (m, 2H), 7.43-7.39 (m, 2H), 7.33 (d, J=2.7 Hz, 1H), 7.27 (dd, J=8.7, 2.7 Hz, 1H), 6.96 (d, J=8.7 Hz, 1H), 5.75-5.60 (m, 1H), 4.62-4.58 (m, 2H), 2.54 (s, 6H), 2.13 (ddd, J=13.9, 3.7, 2.7 Hz, 1H), 1.93 (ddd, J=13.8, 11.1, 3.7 Hz, 1H); MS (APCI⁺) m/z 471 (M+H)⁺.

### Example 138: (2R,4R)-6-chloro-N-{3-[1-(4-chloro-3-fluorophenyl)-1H-pyrazol-4-yl]bicyclo[1.1.1]pentan-1-yl}-4-hydroxy-3,4-dihydro-2H-1-benzopyran-2-carboxamide (Compound 237)

### Example 138A: Tert-Butyl 2-((4-chloro-3-fluorophenyl)amino)acetate

To a solution of 4-chloro-3-fluoroaniline (4.00 g, 27.5 mmol) and tert-butyl 2-bromoacetate (4.46 mL, 30.2 mmol) in N,N-dimethylformamide (30 mL) were added sodium iodide (0.824 g, 5.50 mmol) and N,N-diisopropylethylamine (7.20 mL, 41.2 mmol). The resulting mixture was heated and stirred at 80° C. for 16 hours. The mixture was poured into water (200 mL) and was extracted with ethyl acetate (2×100 mL). The combined organic layers were washed with brine (100 mL), dried (MgSO₄), filtered and concentrated under reduced pressure. The crude product was purified by chromatography on silica gel (0-50% ethyl acetate/isohexane) to afford the title compound (6.65 g, 88% yield). ¹H NMR (500 MHz, DMSO-d₆) δ ppm 7.20 (t, J=8.7 Hz, 1H), 6.52 (dd, J=12.5, 2.7 Hz, 1H), 6.45-6.36 (m, 2H), 3.80 (d, J=6.3 Hz, 2H), 1.42 (s, 9H); MS (ESI) m/z 204 (M+H−C(CH₃))⁺.

### Example 138B: 2-((4-chloro-3-fluorophenyl)amino)acetic Acid

To a stirred solution of the product of Example 138A (6.64 g, 25.6 mmol) in dioxane (30 mL) was added trifluoroacetic acid (10 mL). The reaction mixture was heated and stirred at 80° C. for 48 hours. The volatiles were evaporated under reduced pressure azeotroping with toluene (2×20 mL). The solid was triturated with isohexane-ethyl acetate (1:1, 50 mL), filtered and dried under vacuum to give the title compound (1.90 g, 33% yield). ¹H NMR (500 MHz, DMSO-d₆) δ ppm 12.65 (s, 1H), 7.20 (t, J=8.8 Hz, 1H), 6.54 (dd, J=12.5, 2.7 Hz, 1H), 6.43 (dd, J=8.8, 2.6 Hz, 1H), 6.38 (s, 1H), 3.83 (s, 2H); MS (ESI) m/z 205 (M+H)⁺.

### Example 138C: 2-((4-chloro-3-fluorophenyl)(nitroso)amino)acetic Acid

To a solution of the product of Example 138B (1.90 g, 9.33 mmol) in water (20 mL) and acetonitrile (10 mL) was added sodium nitrite (0.644 g, 9.33 mmol) and the resulting mixture was stirred at ambient temperature for 16 hours. The solvent was evaporated under reduced pressure to give the title compound (2.24 g, 100% yield). ¹H NMR (500 MHz, DMSO-d₆) δ ppm 7.75-7.59 (m, 2H), 7.47 (ddd, J=8.8, 2.5, 1.1 Hz, 1H), 4.35 (s, 2H), one exchangeable proton not observed; MS (ESI) m/z 231 (M−H)⁻.

### Example 138D: 3-(4-chloro-3-fluorophenyl)-2,3-dihydro-1,2,3-oxadiazol-5-ol

A solution of the product of Example 138C (2.23 g, 9.59 mmol) in acetic anhydride (0.905 mL, 9.59 mmol) was stirred and heated at 100° C. for 2 hours. Then the reaction was concentrated under reduced pressure. The residue was suspended in water and the solid was recovered by filtration. The solid was washed with water (2×10 mL) and dried under vacuum at ambient temperature to give the title compound (1.92 g, 84% yield). ¹H NMR (500 MHz, DMSO-d₆) δ ppm 8.20 (dd, J=9.6, 2.5 Hz, 1H), 8.05-7.97 (m, 1H), 7.92-7.86 (m, 1H), 7.85 (s, 1H), 2 exchangeable protons not observed; MS (ESI) m/z 215 (M+H)⁺.

### Example 138E: Tert-Butyl (3-(f-(4-chloro-3-fluorophenyl)-1H-pyrazol-4-yl)bicyclo[1.1.1]pentan-1-yl)carbamate

A mixture of the product of Example 138D (51 mg, 0.246 mmol), 4,7-diphenyl-1,10-phenanthroline (16.36 mg, 0.049 mmol), the product of Example 151A (53.3 mg, 0.246 mmol), copper (II) sulfate (7.85 mg, 0.049 mmol) and triethylamine (137 μl, 0.984 mmol) in tert-butanol and water (1:1, 2 mL) was stirred and heated at 60° C. for 2 hours. The mixture was absorbed on silica and purified by chromatography on silica gel (0-30% MTBE/isohexane) to afford the title product (82 mg, 78% yield) as a yellow solid: ¹H NMR (500 MHz, DMSO-d₆) δ ppm 8.43 (s, 1H), 7.90 (dd, J=11.0, 2.2 Hz, 1H), 7.75-7.67 (m, 2H), 7.66 (s, 1H), 7.57 (s, 1H), 2.14 (d, J=7.8 Hz, 6H), 1.40 (s, 9H); MS (ESI) m/z 378 (M+H)⁺.

### Example 138F: (2R,4R)-6-chloro-N-{3-[1-(4-chloro-3-fluorophenyl)-1H-pyrazol-4-yl]bicyclo[1.1.1]pentan-1-yl}-4-hydroxy-3,4-dihydro-1H-1-benzopyran-2-carboxamide

The title compound was prepared using the methods described for the synthesis of Example 131D, substituting the product from Example 131C with the product from Example 138E and substituting the product from Example 73B with the product from Example 3B. ¹H NMR (500 MHz, DMSO-d₆) δ ppm 8.72 (s, 1H), 8.48 (s, 1H), 7.92 (dd, J=10.8, 2.3 Hz, 1H), 7.75-7.68 (m, 3H), 7.42-7.37 (m, 1H), 7.21 (dd, J=8.7, 2.7 Hz, 1H), 6.90 (d, J=8.7 Hz, 1H), 5.71 (d, J=6.4 Hz, 1H), 4.87-4.78 (m, 1H), 4.62 (dd, J=12.0, 2.3 Hz, 1H), 2.42-2.35 (m, 1H), 2.30 (s, 6H), 1.77-1.67 (m, 1H); MS (ESI) m/z 488 (M+H)⁺.

### Example 139: (2S,4R)-6-chloro-N-{3-[1-(4-chloro-3-fluorophenyl)-1H-pyrazol-4-yl]bicyclo[1.1.1]pentan-1-yl}-4-hydroxy-3,4-dihydro-2H-1-benzopyran-2-carboxamide (Compound 238)

The title compound was prepared using the methods described for the synthesis of Example 131D, substituting the product from Example 131C with the product from Example 138E. ¹H NMR (500 MHz, DMSO-d₆) δ ppm 8.78 (s, 1H), 8.48 (s, 1H), 7.92 (dd, J=11.0, 2.3 Hz, 1H), 7.76-7.66 (m, 3H), 7.33 (d, J=2.6 Hz, 1H), 7.26 (dd, J=8.7, 2.7 Hz, 1H), 6.95 (d, J=8.7 Hz, 1H), 5.63 (s, 1H), 4.63-4.54 (m, 2H), 2.30 (s, 6H), 2.16-2.08 (m, 1H), 1.97-1.88 (m, 1H); MS (ESI) m/z 488 (M+H)⁺.

### Example 140: (2S,4R)-6-chloro-4-hydroxy-N-[4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 239)

The reaction and purification conditions described in Example 1C substituting the product of Example 90A for the product of Example 1A, and the product of Example 73B for the product of Example 1B gave the title compound. ¹H NMR (400 MHz, DMSO-d₆) δ ppm 7.38 (s, 1H), 7.30 (d, J=2.7 Hz, 1H), 7.22 (dd, J=8.8, 2.7 Hz, 1H), 6.98 (s, 1H), 6.90 (d, J=8.8 Hz, 1H), 5.59 (d, J=4.2 Hz, 1H), 4.60-4.51 (m, 2H), 4.47 (p, J=7.1 Hz, 1H), 3.69 (p, J=6.9 Hz, 1H), 3.68 (s, 2H), 2.78-2.67 (m, 2H), 2.17-2.06 (m, 2H), 2.05-1.97 (m, 1H), 1.97-1.90 (m, 1H), 1.93-1.88 (m, 12H); MS (APCI⁺) m/z 547 (M+H)⁺.

### Example 141: (2R,4R)-6-chloro-N-{3-[3-(4-chlorophenyl)-1H-pyrrol-1-yl]bicyclo[1.1.1]pentan-1-yl}-4-hydroxy-3,4-dihydro-2H-1-benzopyran-2-carboxamide (Compound 240)

### Example 141A: Tert-Butyl (3-(1H-pyrrol-1-yl)bicyclo[1.1.1]pentan-1-yl)carbamate

tert-Butyl (3-aminobicyclo[1.1.1]pentan-1-yl)carbamate (2 g, 10.09 mmol), 2,5-dimethoxytetrahydrofuran (2.2 mL, 17.15 mmol) in a mixture of acetic acid (4 mL) and water (4 mL) was heated to 100° C. for 10 minutes. The reaction mixture was cooled down to ambient temperature and 2 M aqueous NaOH (10 mL) and ethyl acetate (10 mL) were added. The layers were separated, and the organic layer was washed with a saturated aqueous solution of NaHCO₃ (10 mL). The organic layer was dried (MgSO₄), filtered and concentrated in vacuo. The residue was purified by chromatography on silica gel (0-50% ethyl acetate/isohexane) to afford the title compound (2.0 g, 78% yield). ¹H NMR (500 MHz, DMSO-d₆) δ ppm 6.75 (t, J=2.1 Hz, 2H), 6.02 (t, J=2.1 Hz, 2H), 2.30 (s, 6H), 1.40 (s, 9H), one exchangeable not observed; MS (ESI) m/z 249 (M+H)⁺.

### Example 141B: Tert-Butyl (3-(3-bromo-1H-pyrrol-1-yl)bicyclo[1.1.1]pentan-1-yl)carbamate

To the product of Example 141A (1 g, 4.03 mmol) in dichloromethane (10 mL) at −78° C. was added N-bromosuccinimide (NBS, 0.717 g, 4.03 mmol) in dichloromethane (15 mL). The reaction mixture was stirred at −78° C. for 1 hour then at ambient temperature for 30 minutes. The solvent was removed under vacuum. The crude product was purified by chromatography on silica gel (0-60% ethyl acetate/isohexane) to afford the title compound (1.1 g, 67% yield). ¹H NMR (500 MHz, CDCl₃) δ ppm 6.71-6.65 (m, 1H), 6.60-6.54 (m, 1H), 6.22-6.15 (m, 1H), 5.03 (s, 1H), 2.43 (s, 6H), 1.48 (s, 9H); MS (ESI) m/z 327 (M+H)⁺.

### Example 141C: Tert-Butyl (3-(3-(4-chlorophenyl)-1H-pyrrol-1-yl)bicyclo[1.1.1]pentan-1-yl)carbamate

A suspension of the product of Example 141B (350 mg, 1.070 mmol), (4-chlorophenyl)boronic acid (251 mg, 1.604 mmol), and sodium carbonate (227 mg, 2.139 mmol) in a mixture of dioxane (5 mL) and water (2 mL) was degassed under vacuum followed by a nitrogen back flush. Bis(1,2-bis(diphenylphosphino)ethane)palladium (19.32 mg, 0.021 mmol) was added and the reaction mixture was further degassed under vacuum and followed by a nitrogen back flush. The reaction mixture was heated to 80° C. for 50 minutes. Water (15 mL) and ethyl acetate (15 mL) were added and the layers were separated. The organic layer was washed with water (5 mL). The organic layer was dried over MgSO₄, filtered and concentrated in vacuo. The residue was purified by chromatography on silica gel (0-30% ethyl acetate/isohexane) to afford the title compound (105 mg, 26% yield). ¹H NMR (500 MHz, DMSO-d₆) δ ppm 7.73 (s, 1H), 7.57-7.50 (m, 2H), 7.37-7.32 (m, 2H), 7.32-7.28 (m, 1H), 6.82 (dd, J=2.8, 2.2 Hz, 1H), 6.46 (dd, J=2.9, 1.8 Hz, 1H), 2.34 (s, 6H), 1.41 (s, 9H); MS (ESI) m/z 359 (M+H)⁺.

### Example 141D: 3-(3-(4-chlorphenyl)-1H-pyrrol-1-yl)bicyclo[1.1.1]pentan-1-amine, Trifluoroacetic Acid

To the product of Example 141C (102 mg, 0.284 mmol) in dichloromethane (4 mL) was added trifluoroacetic acid (0.328 mL, 4.26 mmol) at ambient temperature. The reaction mixture was stirred at ambient temperature for 2 hours. The volatiles were removed under vacuum and coevaporated with toluene (3×5 mL) to afford the title compound (115 mg, 100% yield). ¹H NMR (500 MHz, DMSO-d₆) δ ppm 8.77 (s, 3H), 7.58-7.52 (m, 2H), 7.41-7.32 (m, 3H), 6.90 (t, J=2.5 Hz, 1H), 6.51 (dd, J=2.9, 1.8 Hz, 1H), 2.46 (s, 6H); MS (ESI) m/z 259 (M+H)⁺.

### Example 141E: (2R,4R)-6-chloro-N-{3-[3-(4-chlorophenyl)-1H-pyrrol-1-yl]bicyclo[1.1.1]pentan-1-yl}-4-hydroxy-3,4-dihydro-2H-1-benzopyran-2-carboxamide

The product of Example 3B (25 mg, 0.109 mmol) and the product from Example 141D (52 mg, 0.139 mmol) were dissolved in anhydrous N,N-dimethylformamide (1 mL) at room temperature. N,N-Diisopropylethylamine (0.134 mL, 0.765 mmol) and 2,4,6-tripropyl-1,3,5,2,4,6-trioxatriphosphinane 2,4,6-trioxide in N,N-dimethylformamide (50%) (0.076 mL, 0.131 mmol) were added and the reaction mixture was stirred at ambient temperature for 16 hours. The reaction mixture was purified by preparative HPLC [Waters XBridge™ C18 5 μm OBD column, 19×50 mm, 50-80% gradient of acetonitrile in buffer (0.1% aqueous ammonium bicarbonate)] to afford the title compound (19 mg, 36% yield). ¹H NMR (500 MHz, DMSO-d₆) δ ppm 8.88 (s, 1H), 7.59-7.53 (m, 2H), 7.42-7.38 (m, 1H), 7.37-7.30 (m, 3H), 7.22 (dd, J=8.7, 2.7 Hz, 1H), 6.91 (d, J=8.7 Hz, 1H), 6.87 (t, J=2.5 Hz, 1H), 6.48 (dd, J=2.9, 1.8 Hz, 1H), 5.72 (d, J=6.3 Hz, 1H), 4.88-4.79 (m, 1H), 4.66 (dd, J=12.0, 2.3 Hz, 1H), 2.49 (s, 6H), 2.43-2.36 (m, 1H), 1.79-1.68 (m, 1H); MS (ESI) m/z 469 (M+H)⁺.

### Example 142: (2S,4R)-6-chloro-N-{3-[3-(4-chlorophenyl)-1H-pyrrol-1-yl]bicyclo[1.1.1]pentan-1-yl}-4-hydroxy-3,4-dihydro-2H-1-benzopyran-2-carboxamide (Compound 241)

The title compound was prepared using the method described for the synthesis of Example 141E, substituting the product of Example 73B for the product of Example 3B. ¹H NMR (500 MHz, DMSO-d₆) δ ppm 8.93 (s, 1H), 7.59-7.52 (m, 2H), 7.36-7.32 (m, 4H), 7.27 (dd, J=8.7, 2.7 Hz, 1H), 6.96 (d, J=8.7 Hz, 1H), 6.86 (t, J=2.5 Hz, 1H), 6.48 (dd, J=2.9, 1.8 Hz, 1H), 5.64 (d, J=4.7 Hz, 1H), 4.64-4.57 (m, 2H), 2.48 (s, 6H), 2.13 (dt, J=13.9, 3.4 Hz, 1H), 1.93 (ddd, J=14.3, 10.9, 3.7 Hz, 1H); MS (ESI) m/z 469 (M+H)⁺.

### Example 143: (2R,4R)-6-chloro-N-{3-[3-(4-chloro-3-fluorophenyl)-1H-pyrrol-1-yl]bicyclo[1.1.1]pentan-1-yl}-4-hydroxy-3,4-dihydro-2H-1-benzopyran-2-carboxamide (Compound 242)

### Example 143A: Tert-Butyl (3-(3-(4-chloro-3-fluorophenyl)-1H-pyrrol-1-yl)bicyclo[1.1.1]pentan-1-yl)carbamate

The title compound was prepared using the method described for the synthesis of Example 141C, substituting (4-chloro-3-fluorophenyl)boronic acid for (4-chlorophenyl)boronic acid. ¹H NMR (500 MHz, DMSO-d₆) δ ppm 7.71 (s, 1H), 7.57 (dd, J=11.3, 2.0 Hz, 1H), 7.50-7.43 (m, 1H), 7.43-7.36 (m, 2H), 6.84 (dd, J=2.9, 2.2 Hz, 1H), 6.53 (dd, J=2.9, 1.8 Hz, 1H), 2.34 (s, 6H), 1.41 (s, 9H); MS (ESI) m/z 377 (M+H)⁺.

### Example 143B: 3-(3-(4-chloro-3-fluorophenyl)-1H-pyrrol-1-yl)bicyclo[1.1.1]pentan-1-amine, Trifluoroacetic Acid

The title compound was prepared using the method described for the synthesis of Example 141D, substituting product of Example 141C with the product of Example 143A. ¹H NMR (500 MHz, DMSO-d₆) δ ppm 8.77 (s, 3H), 7.58 (dd, J=11.3, 2.0 Hz, 1H), 7.52-7.46 (m, 2H), 7.40 (dd, J=8.5, 2.0 Hz, 1H), 6.92 (t, J=2.5 Hz, 1H), 6.57 (dd, J=2.9, 1.8 Hz, 1H), 2.46 (s, 6H); MS (ESI) m/z 277 (M+H)⁺.

### Example 143C: (2R,4R)-6-chloro-N-{3-[3-(4-chloro-3-fluorophenyl)-1H-pyrrol-1-yl]bicyclo[1.1.1]pentan-1-yl}-4-hydroxy-3,4-dihydro-2H-1-benzopyran-2-carboxamide

The title compound was prepared using the method described for the synthesis of Example 141E, substituting product of Example 141D with the product of Example 143B. ¹H NMR (500 MHz, DMSO-d₆) δ ppm 8.88 (s, 1H), 7.59 (dd, J=11.3, 2.0 Hz, 1H), 7.51-7.44 (m, 2H), 7.44-7.38 (m, 2H), 7.22 (dd, J=8.7, 2.7 Hz, 1H), 6.91 (d, J=8.7 Hz, 1H), 6.88 (t, J=2.5 Hz, 1H), 6.55 (dd, J=2.9, 1.8 Hz, 1H), 5.72 (d, J=6.3 Hz, 1H), 4.87-4.79 (m, 1H), 4.66 (dd, J=12.0, 2.3 Hz, 1H), 2.49 (s, 6H), 2.42-2.35 (m, 1H), 1.74 (td, J=12.6, 10.9 Hz, 1H); MS (ESI) m/z 487 (M+H)⁺.

### Example 144: (2S,4R)-6-chloro-N-{3-[3-(4-chloro-3-fluorophenyl)-1H-pyrrol-1-yl]bicyclo[1.1.1]pentan-1-yl}-4-hydroxy-3,4-dihydro-2H-1-benzopyran-2-carboxamide (Compound 243)

The title compound was prepared using the methods described for the synthesis of Example 141E, substituting the product of Example 141D with the product of Example 143B, and substituting the product of Example 3B with the product of Example 73B. ¹H NMR (500 MHz, DMSO-d₆) δ ppm 8.94 (s, 1H), 7.59 (dd, J=11.3, 2.0 Hz, 1H), 7.51-7.43 (m, 2H), 7.41 (dd, J=8.4, 2.0 Hz, 1H), 7.33 (d, J=2.6 Hz, 1H), 7.27 (dd, J=8.7, 2.7 Hz, 1H), 6.96 (d, J=8.7 Hz, 1H), 6.88 (t, 1H), 6.54 (dd, J=2.9, 1.8 Hz, 1H), 5.64 (d, J=4.7 Hz, 1H), 4.64-4.57 (m, 2H), 2.48 (s, 6H), 2.13 (dt, J=13.8, 3.3 Hz, 1H), 1.93 (ddd, J=14.2, 11.0, 3.7 Hz, 1H); MS (ESI) m/z 487 (M+H)⁺.

### Example 145: (2R,4R)-6-chloro-4-hydroxy-N-(3-{3-[6-(trifluoromethyl)pyridin-3-yl]-1H-pyrrol-1-yl}bicyclo[1.1.1]pentan-1-yl)-3,4-dihydro-2H-1-benzopyran-2-carboxamide (Compound 244)

### Example 145A: Tert-Butyl (3-(3-(6-(trifluoromethyl)pyridin-3-yl)-1H-pyrrol-1-yl)bicyclo[1.1.1]pentan-1-yl)carbamate

The title compound was prepared using the method described for the synthesis of Example 141C, substituting (4-chlorophenyl)boronic acid with (6-(trifluoromethyl)pyridin-3-yl)boronic acid. ¹H NMR (500 MHz, DMSO-d₆) δ ppm 8.97 (d, J=2.2 Hz, 1H), 8.15 (dd, J=8.2, 2.2 Hz, 1H), 7.82-7.77 (m, 1H), 7.75 (s, 1H), 7.59 (t, J=2.0 Hz, 1H), 6.93 (dd, J=2.9, 2.1 Hz, 1H), 6.67 (dd, J=2.9, 1.8 Hz, 1H), 2.37 (s, 6H), 1.41 (s, 9H); MS (ESI) m/z 394 (M+H)⁺.

### Example 145B: 3-(3-(6-(trifluoromethyl)pyridin-3-yl)-1H-pyrrol-1-yl)bicyclo[1.1.1]pentan-1-amine, Trifluoroacetic Acid

The title compound was prepared using the method described for the synthesis of Example 141D, substituting product of Example 141C with the product of Example 145A. ¹H NMR (500 MHz, DMSO-d₆) δ ppm 8.97 (d, J=2.2 Hz, 1H), 8.85 (s, 3H), 8.16 (dd, J=8.2, 2.2 Hz, 1H), 7.82 (d, J=8.2 Hz, 1H), 7.67 (t, J=2.0 Hz, 1H), 7.01 (dd, J=2.9, 2.1 Hz, 1H), 6.71 (dd, J=2.9, 1.8 Hz, 1H), 2.49 (s, 6H); MS (ESI) m/z 294 (M+H)⁺.

### Example 145C: (2R,4R)-6-chloro-4-hydroxy-N-(3-{3-[6-(trifluoromethyl)pyridin-3-yl]-1H-pyrrol-1-yl}bicyclo[1.1.1]pentan-1-yl)-3,4-dihydro-2H-1-benzopyran-2-carboxamide

The title compound was prepared using the method described for the synthesis of Example 141E, substituting product of Example 141D with the product of Example 145B. ¹H NMR (500 MHz, DMSO-d₆) δ ppm 8.98 (d, J=2.2 Hz, 1H), 8.89 (s, 1H), 8.17 (dd, J=8.0, 2.2 Hz, 1H), 7.80 (d, J=8.2 Hz, 1H), 7.64 (t, J=2.0 Hz, 1H), 7.42-7.38 (m, 1H), 7.22 (dd, J=8.7, 2.7 Hz, 1H), 7.00-6.95 (m, 1H), 6.91 (d, J=8.7 Hz, 1H), 6.69 (dd, J=2.9, 1.8 Hz, 1H), 5.73 (d, J=6.3 Hz, 1H), 4.88-4.80 (m, 1H), 4.66 (dd, J=12.0, 2.3 Hz, 1H), 2.52 (s, 6H), 2.43-2.35 (m, 1H), 1.79-1.69 (m, 1H); MS (ESI) m/z 504 (M+H)⁺.

### Example 146: (2S,4R)-6-chloro-4-hydroxy-N-(3-{3-[6-(trifluoromethyl)pyridin-3-yl]-1H-pyrrol-1-yl}bicyclo[1.1.1]pentan-1-yl)-3,4-dihydro-2H-1-benzopyran-2-carboxamide (Compound 245)

The title compound was prepared using the methods described for the synthesis of Example 141E, substituting the product from Example 141D with the product of Example 145B, and substituting the product of Example 3B with the product of Example 73B. ¹H NMR (500 MHz, DMSO-d₆) δ ppm 8.98 (d, J=2.2 Hz, 1H), 8.95 (s, 1H), 8.17 (dd, J=8.1, 2.2 Hz, 1H), 7.80 (d, J=8.2 Hz, 1H), 7.64 (t, J=2.0 Hz, 1H), 7.34 (d, J=2.7 Hz, 1H), 7.27 (dd, J=8.7, 2.7 Hz, 1H), 7.00-6.94 (m, 2H), 6.69 (dd, J=2.9, 1.8 Hz, 1H), 5.64 (d, J=4.5 Hz, 1H), 4.64-4.58 (m, 2H), 2.51 (s, 6H), 2.17-2.07 (m, 1H), 1.98-1.89 (m, 1H); MS (ESI) m/z 504 (M+H)⁺.

### Example 147: (2R,4R)-6-chloro-N-{3-[3-(4-chlorophenyl)-1,2-oxazol-5-yl]bicyclo[11.11]pentan-1-yl}-4-hydroxy-3,4-dihydro-2H-1-benzopyran-2-carboxamide (Compound 246)

### Example 147A: 4-chlorobenzaldehyde Oxime

The title compound was prepared using the method described for synthesis of Example 151B, substituting 4-chlorobenzaldehyde for 6-(trifluoromethyl)nicotinaldehyde. ¹H NMR (500 MHz, DMSO-d₆) δ ppm 11.35 (s, 1H), 8.15 (s, 1H), 7.64-7.58 (m, 2H), 7.49-7.42 (m, 2H).

### Example 147B: Tert-Butyl (3-(3-(4-chlorophenyl)isoxazol-5-yl)bicyclo[1.1.1]pentan-1-yl)carbamate

The title compound was prepared using the methods described for the synthesis of Example 128H, substituting the product from Example 147A for the product of Example 128C, and substituting the product from Example 151A for the product from Example 128G. MS (ESI) m/z 361 (M+H)⁺.

### Example 147C: (2R,4R)-6-chloro-N-{3-[3-(4-chlorophenyl)-1,2-oxazol-5-yl]bicyclo[1.1.1]pentan-1-yl}-4-hydroxy-3,4-dihydro-2H-1-benzopyran-2-carboxamide

The title compound was prepared using the methods described for the synthesis of Example 131D, substituting the product from Example 147B for the product from Example 131C, and substituting the product from Example 3B for the product from Example 73B. ¹H NMR (500 MHz, DMSO-d₆) δ ppm 8.86 (s, 1H), 7.89-7.86 (m, 2H), 7.60-7.56 (m, 2H), 7.39 (dd, J=3.0, 1.0 Hz, 1H), 7.21 (ddd, J=8.5, 3.0, 1.0 Hz, 1H), 6.98 (s, 1H), 6.89 (d, J=8.5 Hz, 1H), 5.71 (d, J=6.5 Hz, 1H), 4.86-4.77 (m, 1H), 4.63 (dd, J=12.0, 2.5 Hz, 1H), 2.47 (s, 6H), 2.41-2.34 (m, 1H), 1.77-1.67 (m, 1H); MS (ESI) m/z 469 (M−H)⁻.

### Example 148: (2S,4R)-6-chloro-N-{3-[3-(4-chlorophenyl)-1,2-oxazol-5-yl]bicyclo[1.1.1]pentan-1-yl}-4-hydroxy-3,4-dihydro-2H-1-benzopyran-2-carboxamide (Compound 247)

The title compound was prepared using the methods described for the synthesis of Example 131D, substituting the product from Example 147B for the product from Example 131C. ¹H NMR (500 MHz, DMSO-d₆) δ ppm 8.92 (s, 1H), 7.90-7.85 (m, 2H), 7.60-7.55 (m, 2H), 7.32 (d, J=2.5 Hz, 1H), 7.26 (dd, J=9.0, 2.5 Hz, 1H), 6.98 (s, 1H), 6.94 (d, J=8.5 Hz, 1H), 5.63 (d, J=4.5 Hz, 1H), 4.62-4.55 (m, 2H), 2.46 (s, 6H), 2.15-2.09 (m, 1H), 1.96-1.88 (m, 1H); MS (ESI) m/z 469 (M−H)⁻.

### Example 149: (2R,4R)-6-chloro-N-{3-[3-(4-chloro-3-fluorophenyl)-1,2-oxazol-5-yl]bicyclo[1.1.1]pentan-1-yl}-4-hydroxy-3,4-dihydro-2H-1-benzopyran-2-carboxamide (Compound 248)

### Example 149A: 4-chloro-3-fluorobenzaldehyde Oxime

The title compound was prepared using the method described for synthesis of Example 151B, substituting 4-chloro-3-fluorobenzaldehyde for 6-(trifluoromethyl)nicotinaldehyde. ¹H NMR (500 MHz, DMSO-d₆) δ ppm 11.55 (s, 1H), 8.16 (s, 1H), 7.65-7.55 (m, 2H), 7.46 (dd, J=8.0, 2.0 Hz, 1H).

### Example 149B: Tert-Butyl (3-(3-(4-chloro-3-fluorophenyl)isoxazol-5-yl)bicyclo[1.1.1]pentan-1-yl)carbamate

The title compound was prepared using the methods described for the synthesis of Example 128H, substituting the product from Example 149A for the product of Example 128C, and substituting the product from Example 151A for the product from Example 128G. MS (ESI) m/z 379 (M+H)⁺.

### Example 149C: (2R,4R)-6-chloro-N-{3-[3-(4-chloro-3-fluorophenyl)-1,2-oxazol-5-yl]bicyclo[1.1.1]pentan-1-yl}-4-hydroxy-3,4-dihydro-2H-1-benzopyran-2-carboxamide

The title compound was prepared using the methods described for the synthesis of Example 131D, substituting the product from Example 149B for the product from Example 131C, and substituting the product from Example 3B for the product from Example 73B. ¹H NMR (500 MHz, DMSO-d₆) δ ppm 8.86 (s, 1H), 7.93-7.87 (m, 1H), 7.78-7.72 (m, 2H), 7.39 (dd, J=2.5, 1.0 Hz, 1H), 7.21 (ddd, J=8.5, 2.5, 1.0 Hz, 1H), 7.04 (s, 1H), 6.89 (d, J=8.5 Hz, 1H), 5.72 (d, J=5.5 Hz, 1H), 4.87-4.77 (m, 1H), 4.63 (dd, J=12.0, 2.5 Hz, 1H), 2.47 (s, 6H), 2.41-2.35 (m, 1H), 1.76-1.67 (m, 1H); MS (ESI) m/z 487 (M−H)⁻.

### Example 150: (2S,4R)-6-chloro-N-{3-[3-(4-chloro-3-fluorophenyl)-1,2-oxazol-5-yl]bicyclo[11.11]pentan-1-yl}-4-hydroxy-3,4-dihydro-2H-1-benzopyran-2-carboxamide (Compound 249)

The title compound was prepared using the methods described for the synthesis of Example 131D, substituting the product from Example 149B for the product from Example 131C. ¹H NMR (500 MHz, DMSO-d₆) δ ppm 8.92 (s, 1H), 7.92-7.88 (m, 1H), 7.77-7.72 (m, 2H), 7.32 (d, J=2.5 Hz, 1H), 7.26 (dd, J=8.5, 2.5 Hz, 1H), 7.04 (s, 1H), 6.94 (d, J=8.5 Hz, 1H), 5.63 (d, J=4.5 Hz, 1H), 4.62-4.55 (m, 2H), 2.47 (s, 6H), 2.16-2.08 (m, 1H), 1.97-1.87 (m, 1H); MS (ESI) m/z 487 (M−H)⁻.

### Example 151: (2R,4R)-6-chloro-4-hydroxy-N-(3-{3-[6-(trifluoromethyl)pyridin-3-yl]-1,2-oxazol-5-yl}bicyclo[1.1.1]pentan-1-yl)-3,4-dihydro-2H-1-benzopyran-2-carboxamide (Compound 250)

### Example 151A: Tert-Butyl (3-ethynlbicyclo[1.1.1]pentan-1-yl)carbamate

The title compound (0.70 g, 74%) was prepared using the method described for the synthesis of Example 135A, substituting the product from Example 128B (1.29 g, 4.52 mmol) for the product from Example 128F. ¹H NMR (500 MHz, DMSO-d₆) δ ppm 7.61 (s, 1H), 3.10 (s, 1H), 2.14 (s, 6H), 1.37 (s, 9H).

### Example 151B: 6-(trifluoromethyl)nicotinaldehyde Oxime

To a solution of 6-(trifluoromethyl)nicotinaldehyde (1.00 g, 5.71 mmol) in a mixed solvent of ethanol (25 mL) and water (2.78 mL) was added hydroxylamine hydrochloride (2.381 g, 34.3 mmol) and sodium acetate (2.81 g, 34.3 mmol), and the resulting mixture was stirred at 80° C. for 16 hours. The mixture was diluted with ethyl acetate (50 mL) and washed with water (30 mL), and the aqueous phase was extracted with ethyl acetate (75 mL 2). The combined organic extract was dried via hydrophobic frit and concentrated in vacuo to give the title compound (1.54 g, 5.67 mmol, 99% yield).

### Example 151C: Tert-Butyl (3-(3-(6-(trifluoromethyl)pyridin-3-yl)isoxazol-5-yl)bicyclo[1.1.1]pentan-1-yl)carbamate

The title compound was prepared using the methods described for the synthesis of Example 128H, substituting the product from Example 151B for the product of Example 128C, and substituting the product from Example 151A for the product from Example 128G. MS (ESI) m/z 396 (M+H)⁺.

### Example 151D: (2R,4R)-6-chloro-4-hydroxy-N-(3-{3-[6-(trifluoromethyl)pyridin-3-yl]-1,2-oxazol-5-yl}bicyclo[1.1.1]pentan-1-yl)-3,4-dihydro-2H-1-benzopyran-2-carboxamide

The title compound was prepared using the methods described for the synthesis of Example 131D, substituting the product from Example 151C for the product from Example 131C, and substituting the product from Example 3B for the product from Example 73B. ¹H NMR (500 MHz, DMSO-d₆) δ ppm 9.24 (d, J=2.0 Hz, 1H), 8.88 (s, 1H), 8.54 (dd, J=8.0, 2.0 Hz, 1H), 8.08 (d, J=8.5 Hz, 1H), 7.39 (dd, J=3.0, 1.0 Hz, 1H), 7.21 (dd, J=8.5, 2.5 Hz, 1H), 7.19 (s, 1H), 6.90 (d, J=8.5 Hz, 1H), 5.72 (d, J=5.5 Hz, 1H), 4.87-4.78 (m, 1H), 4.63 (dd, J=12.0, 2.5 Hz, 1H), 2.50 (s, 6H), 2.42-2.34 (m, 1H), 1.77-1.67 (m, 1H); MS (ESI) ma 504 (M−H)⁻.

### Example 152: (2S,4R)-6-chloro-4-hydroxy-N-(3-{3-[6-(trifluoromethyl)pyridin-3-yl]-1,2-oxazol-5-yl}bicyclo[1.1.1]pentan-1-yl)-3,4-dihydro-2H-1-benzopyran-2-carboxamide (Compound 251)

The title compound was prepared using the methods described for the synthesis of Example 131D, substituting the product from Example 151C for the product from Example 131C. ¹H NMR (500 MHz, DMSO-d₆) δ ppm 9.23 (d, J=2.0 Hz, 1H), 8.94 (s, 1H), 8.55-8.51 (m, 1H), 8.08 (dd, J=8.0, 1.0 Hz, 1H), 7.33 (d, J=2.5 Hz, 1H), 7.26 (dd, J=8.5, 2.5 Hz, 1H), 7.18 (s, 1H), 6.95 (d, J=8.5 Hz, 1H), 5.64 (s, 1H), 4.62-4.56 (m, 2H), 2.49 (s, 6H), 2.15-2.09 (m, 1H), 1.96-1.88 (m, 1H); MS (ESI) m/z 504 (M−H)⁻.

### Example 153: 2-(4-chloro-3-fluorophenoxy)-N-(3-{5-[(2R*,4R*)-6-chloro-4-hydroxy-3,4-dihydro-2H-1-benzopyran-2-yl]-1,3,4-oxadiazol-2-yl}bicyclo[1.1.1]pentan-1-yl)acetamide (Compound 252)

Example 67 was purified by chiral SFC (supercritical fluid chromatography) using a Chiralcel® OD-H, 250×21 mm I.D., 5 μm column eluting with 100% CH₃OH in CO₂ with a flow rate of 80 g/minute and back pressure of 100 bar to give the title compound (second isomer eluted out of the column). The stereochemistry of this title compound was arbitrarily assigned (This compound is the enantiomer of Example 154). ¹H NMR (600 MHz, DMSO-de) δ ppm 8.94 (s, 1H), 7.51 (t, J=8.9 Hz, 1H), 7.43 (dd, J=2.7, 1.0 Hz, 1H), 7.21 (ddd, J=8.7, 2.7, 0.7 Hz, 1H), 7.09 (dd, J=11.3, 2.8 Hz, 1H), 6.90-6.84 (m, 2H), 5.80 (d, J=6.3 Hz, 1H), 5.69 (dd, J=11.5, 2.3 Hz, 1H), 4.91 (dt, J=11.2, 5.9 Hz, 1H), 4.51 (s, 2H), 2.54 (ddd, J=13.2, 6.0, 2.4 Hz, 1H), 2.51 (s, 6H), 2.15 (ddd, J=13.1, 11.6, 10.4 Hz, 1H); MS (APCI⁺) m/z 521 (M+H)⁺.

### Example 154: 2-(4-chloro-3-fluorophenoxy)-N-(3-{5-[(2S*,4S*)-6-chloro-4-hydroxy-3,4-dihydro-2H-1-benzopyran-2-yl]-1,3,4-oxadiazol-2-yl}bicyclo[1.1.1]pentan-1-yl)acetamide (Compound 253)

Example 67 was purified by chiral SFC (supercritical fluid chromatography) using a Chiralcel® OD-H, 250×21 mm I.D., 5 μm column eluting with 20% CH₃OH in CO₂ with a flow rate of 80 g/minute and back pressure of 100 bar to give the impure title compound (first isomer eluted out of the column). This impure residue was further purified by preparative HPLC (Phenomenex® Luna® C8(2) 5 μm AXIA™ column (150 mm×30 mm) using a 30-100% gradient of acetonitrile (A) and 0.1% trifluoroacetic acid in water (B) over 25 minutes, at a flow rate of 50 mL/minute) to isolate the title compound. The stereochemistry of this title compound was arbitrarily assigned (This compound is the enantiomer of Example 153). ¹H NMR (600 MHz, DMSO-d₆) δ ppm 8.95 (s, 1H), 7.50 (td, J=8.9, 2.8 Hz, 1H), 7.42 (dd, J=2.7, 1.0 Hz, 1H), 7.21 (ddd, J=8.7, 2.7, 0.7 Hz, 1H), 7.08 (dd, J=11.3, 2.9 Hz, 1H), 6.89-6.82 (m, 2H), 5.68 (dd, J=11.5, 2.4 Hz, 1H), 4.91 (dd, J=10.3, 5.9 Hz, 1H), 4.51 (s, 2H), 2.57-2.52 (m, 1H), 2.51 (s, 6H), 2.25 (d, J=4.4 Hz, 1H), 2.14 (ddd, J=13.1, 11.5, 10.3 Hz, 1H); MS (APCI⁺) m/z 521 (M+H)⁺.

### Example 155: (2R,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide (Compound 254)

### Example 155A: Tert-Butyl (3-((2-(4-chlorophenyl)-2-oxoethyl)carbamoyl)bicyclo[1.1.1]pentan-1-yl)carbamate

To a solution of 2-amino-1-(4-chlorophenyl)ethanone hydrochloride (Fluorochem, 0.250 g, 1.21 mmol) in N,N-dimethylformamide (10 mL) was added 3-((tert-butoxycarbonyl)amino)bicyclo[1.1.1]pentane-1-carboxylic acid (PharmaBlock, 0.331 g, 1.46 mmol), N,N-diisopropylethylamine (DIPEA, 0.64 mL, 3.6 mmol) and 1-(bis(dimethylamino)methylene)-1H-1,2,3-triazolo[4,5-b]pyridinium 3-oxid hexafluorophosphate (HATU, 0.692 g, 1.82 mmol). The reaction mixture was then stirred at ambient temperature for 19 hours. After this time, the solvent was removed under reduced pressure and the resulting residue was diluted with ethyl acetate (10 mL), washed with HCl (1 M, 3×10 mL), sodium bicarbonate solution (saturated aqueous, 3×10 mL) and brine (3×10 mL). The organic layer was then concentrated in vacuo to give the title intermediate (0.864 g, 1.21 mmol, quantitative yield). ¹H NMR (500 MHz, DMSO-d₆) δ ppm 8.65 (s, 1H), 8.10 (t, J=5.7 Hz, 1H), 7.98 (d, J=8.6 Hz, 2H), 7.61 (d, J=8.6 Hz, 2H), 4.52 (d, J=5.7 Hz, 2H), 2.08 (s, 6H), 1.38 (s, 9H); MS (ESI⁺) m/z 379 (M+H)⁺.

### Example 155B: 3-(5-(4-chlorophenyl)oxazol-2-yl)bicyclo[1.1.1]pentan-1-amine

To the product of Example 155A (200 mg, 0.528 mmol) was added sulfuric acid (500 μL, 9.38 mmol). The reaction mixture was heated at 80° C. for 30 minutes. The reaction mixture was then poured into an ice solution (10 mL) and basified with aqueous ammonia to basic pH. The aqueous layer was extracted with dichloromethane (3×5 mL). The combined organic layers were concentrated in vacuo to afford the title intermediate (72.0 mg, 0.257 mmol, 44% yield). ¹H NMR (500 MHz, DMSO-d₆) δ ppm 7.72-7.66 (m, 2H), 7.61 (s, 1H), 7.56-7.49 (m, 2H), 2.13 (s, 6H); MS (ESI⁺) m/z 261 (M+H)⁺.

### Example 155C: (2R,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide

The product of Example 3B (20 mg, 0.087 mmol) and Example 155B (30 mg, 0.12 mmol) were dissolved in N, N-dimethyl formamide (0.7 mL) at ambient temperature. To this solution were added N, N-diisopropylethylamine (0.11 mL, 0.61 mmol) and 1-propanephosphonic anhydride (T3P®, 50 weight % solution in N,N-dimethylformamide, 0.062 mL, 0.11 mmol) and the reaction mixture was stirred at ambient temperature for 16 hours. The reaction mixture was purified by preparative HPLC [Waters XBridge™ C18 5 μm, 19-50 mm column, 20-65% gradient of acetonitrile in buffer (0.025 M aqueous ammonium bicarbonate, adjusted to pH 10 with ammonium hydroxide)] to give the title compound (13 mg, 0.028 mmol, 32% yield). ¹H NMR (500 MHz, DMSO-d₆) δ ppm 8.87 (s, 1H), 7.72 (d, J=8.6 Hz, 2H), 7.66 (s, 1H), 7.54 (d, J=8.6 Hz, 2H), 7.40 (d, J=2.8 Hz, 1H), 7.22 (dd, J=8.7, 2.8 Hz, 1H), 6.90 (d, J=8.7 Hz, 1H), 5.72 (d, J=6.3 Hz, 1H), 4.84-4.81 (m, 1H), 4.64 (dd, J=12.0, 2.3 Hz, 1H), 2.49 (s, 6H), 2.38-2.36 (m, 1H), 1.74-1.71 (m, 1H); MS (ESI) m/z 471/473 (³⁵Cl/³⁷Cl, M+H)⁺.

### Example 156: (2S,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide (Compound 255)

The methodologies described in Example 155C substituting the product of Example 73B for Example 3B gave the title compound. ¹H NMR (500 MHz, DMSO-d₆) δ ppm 8.92 (s, 1H), 7.77-7.69 (m, 2H), 7.66 (s, 1H), 7.59-7.51 (m, 2H), 7.33 (d, J=2.6 Hz, 1H), 7.27 (dd, J=8.7, 2.7 Hz, 1H), 6.95 (d, J=8.7 Hz, 1H), 5.64 (d, J=4.7 Hz, 1H), 4.63-4.56 (m, 2H), 2.49 (s, 6H), 2.12 (dt, J=14.0, 3.4 Hz, 1H), 1.95-1.90 (m, 1H); MS (ESI+) m/z 471/473 (³⁵Cl/³⁷Cl, M+H)⁺.

### Example 157: (2R,4R)-6-chloro-N-{3-[5-(4-chlorophenyl)-1,2-oxazol-3-yl]bicyclo[1.1.1]pentan-1-yl}-4-hydroxy-3,4-dihydro-2H-1-benzopyran-2-carboxamide (Compound 256)

### Example 157A: Tert-Butyl (3-(5-(4-chlorophenyl)isoxazol-3-yl)bicyclo[1.1.1]pentan-1-yl)carbamate

The title compound was prepared using the methods described for the synthesis of Example 128H, substituting 1-chloro-4-ethynylbenzene for the product from Example 128G. ¹H NMR (500 MHz, DMSO-d₆) δ ppm 7.88-7.82 (m, 2H), 7.69 (s, 1H), 7.63-7.57 (m, 2H), 7.04 (s, 1H), 2.26 (s, 6H), 1.40 (s, 9H).

### Example 157B: (2R,4R)-6-chloro-N-{3-[5-(4-chlorophenyl)-1,2-oxazol-3-yl]bicyclo[1.1.1]pentan-1-yl}-4-hydroxy-3,4-dihydro-2H-1-benzopyran-2-carboxamide

The title compound was prepared using the methods described for the synthesis of Example 131D, substituting the product from Example 157A for the product from Example 131C, and substituting the product from Example 3B for the product from Example 73B. ¹H NMR (500 MHz, DMSO-d₆) δ ppm 8.82 (s, 1H), 7.89-7.82 (m, 2H), 7.64-7.57 (m, 2H), 7.39 (dd, J=2.5, 1.0 Hz, 1H), 7.21 (dd, J=8.5, 2.5 Hz, 1H), 7.08 (s, 1H), 6.90 (d, J=8.5 Hz, 1H), 5.71 (d, J=6.5 Hz, 1H), 4.82 (dt, J=11.5, 6.0 Hz, 1H), 4.63 (dd, J=12.0, 2.0 Hz, 1H), 2.43-2.34 (m, 7H), 1.77-1.67 (m, 1H); MS (ESI) m/z 469 (M−H)⁻.

### Example 158: (2S,4R)-6-chloro-N-{3-[5-(4-chlorophenyl)-1,2-oxazol-3-yl]bicyclo[1.1.1]pentan-1-yl}-4-hydroxy-3,4-dihydro-2H-1-benzopyran-2-carboxamide (Compound 257)

The title compound was prepared using the methods described for the synthesis of Example 131D, substituting the product from Example 157A for the product from Example 131C. ¹H NMR (500 MHz, DMSO-d₆) δ ppm 8.88 (s, 1H), 7.88-7.83 (m, 2H), 7.63-7.58 (m, 2H), 7.32 (d, J=2.5 Hz, 1H), 7.26 (dd, J=8.5, 2.5 Hz, 1H), 7.08 (s, 1H), 6.95 (d, J=8.5 Hz, 1H), 5.63 (d, J=4.5 Hz, 1H), 4.62-4.54 (m, 2H), 2.40 (s, 6H), 2.12 (dt, J=14.0, 3.5 Hz, 1H), 1.96-1.88 (m, 1H); MS (ESI) m/z 469 (M−H)⁻.

### Example 159: (2R,4R)-6-chloro-N-{3-[5-(4-chloro-3-fluorophenyl)-1,2-oxazol-3-yl]bicyclo[1.1.1]pentan-1-yl}-4-hydroxy-3,4-dihydro-2H-1-benzopyran-2-carboxamide (Compound 258)

### Example 159A: 1-chloro-4-ethynyl-2-fluorobenzene

The title compound was prepared using the method described for the synthesis of Example 135A, substituting 4-chloro-3-fluorobenzaldehyde (0.30 g, 1.89 mmol) for the product from Example 128F (0.29 g, 100%).

### Example 159B: Tert-Butyl (3-(5-(4-chloro-3fluorophenyl)isoxazol-3-yl)bicyclo[1.1.1]pentan-1-yl)carbamate

The title compound was prepared using the methods described for the synthesis of Example 128H, substituting the product from Example 159A for the product from Example 128G. ¹H NMR (500 MHz, DMSO-d₆) δ ppm 7.91 (dd, J=10.0, 2.0 Hz, 1H), 7.80-7.74 (m, 1H), 7.70 (dd, J=8.5, 2.0 Hz, 1H), 7.12 (s, 1H), 2.26 (s, 6H), 1.40 (s, 9H).

### Example 159C: (2R,4R)-6-chloro-N-{3-[5-(4-chloro-3-fluorophenyl)-1,2-oxazol-3-yl]bicyclo[1.1.1]pentan-1-yl}-4-hydroxy-3,4-dihydro-2H-1-benzopyran-2-carboxamide

The title compound was prepared using the methods described for the synthesis of Example 131D, substituting the product from Example 159B for the product from Example 131C, and substituting the product from Example 3B for the product from Example 73B. ¹H NMR (500 MHz, DMSO-d₆) δ ppm 8.83 (s, 1H), 7.92 (dd, J=10.0, 2.0 Hz, 1H), 7.78 (app. t, J=8.0 Hz, 1H), 7.71 (dd, J=8.5, 2.0 Hz, 1H), 7.39 (d, J=2.5 Hz, 1H), 7.21 (dd, J=8.5, 2.5 Hz, 1H), 7.17 (s, 1H), 6.90 (d, J=8.5 Hz, 1H), 5.71 (d, J=6.5 Hz, 1H), 4.86-4.79 (m, 1H), 4.63 (dd, J=12.0, 2.5 Hz, 1H), 2.43-2.34 (m, 7H), 1.77-1.66 (m, 1H); MS (ESI) m/z 487 (M−H)⁻.

### Example 160: (2S,4R)-6-chloro-N-{3-[5-(4-chloro-3-fluorophenyl)-1,2-oxazol-3-yl]bicyclo[11.11]pentan-1-yl}-4-hydroxy-3,4-dihydro-2H-1-benzopyran-2-carboxamide (Compound 259)

The title compound was prepared using the methods described for the synthesis of Example 131D, substituting the product from Example 159B for the product from Example 131C. ¹H NMR (500 MHz, DMSO-d₆) δ ppm 8.89 (s, 1H), 7.92 (dd, J=10.0, 2.0 Hz, 1H), 7.80-7.75 (m, 1H), 7.71 (dd, J=8.5, 2.0 Hz, 1H), 7.32 (d, J=2.5 Hz, 1H), 7.26 (dd, J=8.5, 2.5 Hz, 1H), 7.16 (s, 1H), 6.95 (d, J=8.5 Hz, 1H), 5.63 (d, J=4.5 Hz, 1H), 4.62-4.55 (m, 2H), 2.41 (s, 6H), 2.12 (dt, J=14.0, 3.5 Hz, 1H), 1.96-1.87 (m, 1H); MS (ESI) m/z 487 (M−H)⁻.

### Example 161: (2R,4R)-6-chloro-4-hydroxy-N-(3-{5-[6-(trifluoromethyl)pyridin-3-yl]-1,2-oxazol-3-yl}bicyclo[1.1.1]pentan-1-yl)-3,4-dihydro-2H-1-benzopyran-2-carboxamide (Compound 260)

### Example 161A: 5-ethynyl-2-(trifluoromethyl)pyridine

The title compound (0.29 g, 100%) was prepared using the method described for the synthesis of Example 135A, substituting 6-(trifluoromethyl)nicotinaldehyde (0.30 g, 1.71 mmol) for the product from Example 128F.

### Example 161B: Tert-Butyl (3-(5-(6-(trifluoromethyl)pyridin-3-yl)isoxazol-3-yl)bicyclo[1.1.1]pentan-1-yl)carbamate

The title compound was prepared using the methods described for the synthesis of Example 128H, substituting the product from Example 161A for the product from Example 128G. ¹H NMR (500 MHz, DMSO-d₆) δ ppm 9.24 (d, J=2.0 Hz, 1H), 8.50 (dd, J=8.0, 2.0 Hz, 1H), 8.08 (d, J=8.0 Hz, 1H), 7.71 (s, 1H), 7.34 (s, 1H), 2.29 (s, 6H), 1.40 (s, 9H).

### Example 161C: (2R,4R)-6-chloro-4-hydroxy-N-(3-{5-[6-(trifluoromethyl)pyridin-3-yl]-1,2-oxazol-3-yl}bicyclo[1.1.1]pentan-1-yl)-3,4-dihydro-2H-1-benzopyran-2-carboxamide

The title compound was prepared using the methods described for the synthesis of Example 131D, substituting the product from Example 161B for the product from Example 131C, and substituting the product from Example 3B for the product from Example 73B. ¹H NMR (500 MHz, DMSO-d₆) δ ppm 9.25 (d, J=2.0 Hz, 1H), 8.85 (s, 1H), 8.51 (dd, J=8.5, 2.0 Hz, 1H), 8.09 (d, J=8.0 Hz, 1H), 7.41-7.36 (m, 2H), 7.21 (dd, J=8.5, 2.5 Hz, 1H), 6.89 (d, J=8.5 Hz, 1H), 5.71 (d, J=6.0 Hz, 1H), 4.86-4.79 (m, 1H), 4.63 (dd, J=12.0, 2.5 Hz, 1H), 2.44 (s, 6H), 2.40-2.35 (m, 1H), 1.78-1.67 (m, 1H) MS (ESI) m/z 504 (M−H)⁻.

### Example 162: (2S,4R)-6-chloro-4-hydroxy-N-(3-{5-[6-(trifluoromethyl)pyridin-3-yl]-1,2-oxazol-3-yl}bicyclo[1.1.1]pentan-1-yl)-3,4-dihydro-2H-1-benzopyran-2-carboxamide (Compound 261)

The title compound was prepared using the methods described for the synthesis of Example 131D, substituting the product from Example 161B for the product from Example 131C. ¹H NMR (500 MHz, DMSO-d₆) δ ppm 9.24 (d, J=2.0 Hz, 1H), 8.90 (s, 1H), 8.51 (dd, J=8.5, 2.0 Hz, 1H), 8.09 (d, J=8.5 Hz, 1H), 7.38 (s, 1H), 7.33 (d, J=2.5 Hz, 1H), 7.26 (dd, J=8.5, 2.5 Hz, 1H), 6.95 (d, J=8.5 Hz, 1H), 5.63 (d, J=4.5 Hz, 1H), 4.63-4.55 (m, 2H), 2.43 (s, 6H), 2.15-2.09 (m, 1H), 1.96-1.88 (m, 1H); MS (ESI) m/z 504 (M−H)⁻.

### Example 163: (2R,4R)-6-chloro-4-hydroxy-N-(3-{1-[6-(trifluoromethyl)pyridin-3-yl]-1H-pyrazol-4-yl}bicyclo[1.1.1]pentan-1-yl)-3,4-dihydro-2H-1-benzopyran-2-carboxamide (Compound 262)

### Example 163A: 2-((6-(trifluoromethyl)pyridin-3-yl)amino)acetic Acid

At room temperature, sodium iodide (148 mg, 0.986 mmol) was added to a solution of N-ethyl-N-isopropylpropan-2-amine (1288 μl, 7.39 mmol), 6-(trifluoromethyl)pyridin-3-amine (799 mg, 4.93 mmol), and tert-butyl 2-bromoacetate (801 μL, 5.42 mmol) in N,N-dimethylformamide (5.0 mL). The suspension was stirred at 80° C. for 17 hours. Water (50 mL) was added, and the suspension was extracted with ethyl acetate (2×30 mL). The combined organic extracts were washed with brine (30 mL), dried over MgSO₄, filtered and concentrated. The residue was purified by C18 reversed-phase flash chromatography (120 g cartridge, 5-40% acetonitrile/10 mM ammonium bicarbonate) to afford the title compound (457 mg, 1.829 mmol, 37.1% yield). ¹H NMR (500 MHz, DMSO-d₆) δ ppm 8.07 (d, J=2.8 Hz, 1H), 7.51 (d, J=8.6 Hz, 1H), 6.98 (dd, J=8.7 Hz, 2.8 Hz, 1H), 6.79 (br t, 1H), 3.85 (d, J=5.4 Hz, 2H).

### Example 163B: 2-(nitroso(6-(trifluoromethyl)pyridin-3-yl)amino)acetic Acid

At room temperature, sodium nitrite (0.143 g, 2.076 mmol) was added to a suspension of the product of Example 163A (0.457 g, 2.076 mmol) in acetonitrile (2.3 mL) and water (4.6 mL) and the mixture was stirred for 3 hours. Then additional sodium nitrite (0.019 g, 0.415 mmol) was added, and the reaction mixture was stirred for another 30 minutes. The reaction mixture was concentrated to give the title compound (0.573 g, 1.460 mmol, 71% yield). ¹H NMR (500 MHz, DMSO-d₆) δ ppm 9.00 (d, J=2.5 Hz, 1H), 8.22 (dd, J=8.6 Hz, 2.6 Hz, 1H), 8.02 (d, J=8.6 Hz, 1H), 4.42 (s, 2H).

### Example 163C: 3-(6-(trifluoromethyl)pyridin-3-yl)-2,3-dihydro-1,2,3-oxadiazol-5-ol

The suspension of acetic anhydride (5 mL, 53.0 mmol) and Example 163B (573 mg, 2.300 mmol) was stirred at 100° C. for 2 hours, and the reaction mixture was concentrated. Water (50 mL) was added and the suspension was extracted with ethyl acetate (3×50 mL). The combined organic extracts were washed with saturated aqueous NaHCO₃ (50 mL) and brine (50 mL), dried over MgSO₄, filtered and concentrated to give the title compound (366 mg, 1.334 mmol, 58% yield) as an orange/brown solid. ¹H NMR (500 MHz, DMSO-d₆) δ ppm 9.35 (d, J=2.5 Hz, 1H), 8.69 (dd, J=8.5 Hz, 2.5 Hz, 1H), 8.32 (d, J=8.6 Hz, 1H), 7.98 (s, 1H).

### Example 163D: Tert-Butyl (3-(1-(6-(trifluoromethyl)pyridin-3-yl)-1H-pyrazol-4-yl)bicyclo[1.1.1]pentan-1-yl)carbamate

The title compound was synthesized using the same procedure as described in Example 138E substituting the product of Example 138D with the product of Example 163C. ¹H NMR (500 MHz, DMSO-d₆) δ ppm 9.24 (d, J=2.5 Hz, 1H), 8.58 (s, 1H), 8.44 (dd, J=8.6, 2.6 Hz, 1H), 8.02 (d, J=8.6 Hz, 1H), 7.76 (s, 1H), 7.57 (br s, 1H), 2.16 (s, 6H), 1.38 (s, 9H).

### Example 163E: 3-(1-(6-(trifluoromethyl)pyridin-3-yl)-1H-pyrazol-4-yl)bicyclo[1.1.1]pentan-1-amine Trifloroacetic Acid

At room temperature, trifluoroacetic acid (1.0 mL, 12.98 mmol) was added to the product of Example 163D (150 mg, 0.380 mmol). The solution was stirred at room temperature for 90 minutes. Toluene (5 mL) was added and the mixture was concentrated. Toluene (5 mL) was added again and the mixture was concentrated to give the title compound (146 mg, 0.325 mmol, 86% yield). ¹H NMR (500 MHz, DMSO-d₆) δ ppm 9.24 (d, J=2.5 Hz, 1H), 8.70 (br s, 3H), 8.68 (s, 1H), 8.44 (dd, J=8.5, 2.6 Hz, 1H), 8.05 (d, J=8.6 Hz, 1H), 7.84 (s, 1H), 2.26 (s, 6H).

### Example 163F: (2R,4R)-6-chloro-4-hydroxy-N-(3-(1-(6-(trifluoromethyl)pyridin-3-yl)-1H-pyrazol-4-ylbicyclo[1.1.1]pentan-1-yl)-3,4-dihydro-2H-1-benzopyran-2-carboxamide

At room temperature, N,N-diisopropylethylamine (0.107 mL, 0.612 mmol), followed by 2,4,6-tripropyl-1,3,5,2,4,6-trioxatriphosphinane 2,4,6-trioxide (0.061 mL, 0.105 mmol), were added to a solution of the product of Example 3B (20 mg, 0.087 mmol) and the product of Example 163E (41.7 mg, 0.102 mmol) in N,N-dimethylformamide (1.00 mL). The mixture was stirred at room temperature overnight. The reaction mixture was purified by preparative HPLC (Waters XSelect® Prep-C18, 5 μm column (19 mm×50 mm). A 35-65% gradient of 0.1% formic acid in acetonitrile (A) and 0.1% formic acid in water (B) was used over 7.5 minutes, at a flow rate of 30 mL/minute) to give the title compound (18.0 mg, 0.034 mmol, 38.7% yield). ¹H NMR (500 MHz, DMSO-d₆) δ ppm: 9.25 (d, J=2.6 Hz, 1H), 8.72 (s, 1H), 8.62 (s, 1H), 8.45 (dd, J=8.5, 2.6 Hz, 1H), 8.03 (d, J=8.6 Hz, 1H), 7.80 (s, 1H), 7.38 (d, J=2.6 Hz, 1H), 7.20 (dd, J=8.7, 2.7 Hz, 1H), 6.89 (d, J=8.7 Hz, 1H), 5.70 (br s, 1H), 4.83-4.78 (br m, 1H), 4.61 (dd, J=12.0, 2.3 Hz, 1H), 2.39-2.34 (m, 1H), 2.31 (s, 6H), 1.74-1.67 (m, 1H).

### Example 164: (2S,4R)-6-chloro-4-hydroxy-N-(3-{1-[6-(trifluoromethyl)pyridin-3-yl]-1H-pyrazol-4-yl}bicyclo[1.1.1]pentan-1-yl)-3,4-dihydro-2H-1-benzopyran-2-carboxamide (Compound 263)

The title compound was synthesized using the same procedure as described in Example 163F substituting the product of Example 3B with the product of Example 73B. ¹H NMR (500 MHz, DMSO-d₆) δ ppm 9.25 (d, J=2.5 Hz, 1H), 8.78 (s, 1H), 8.62 (s, 1H), 8.45 (dd, J=8.5, 2.6 Hz, 1H), 8.03 (d, J=8.6 Hz, 1H), 7.80 (s, 1H), 7.31 (d, J=2.7 Hz, 1H), 7.25 (dd, J=8.7, 2.7 Hz, 1H), 6.94 (d, J=8.7 Hz, 1H), 5.61 (brs, 1H), 4.59 (br t, J=3.7 Hz, 1H), 4.56 (dd, J=11.0, 2.7 Hz, 1H), 2.31 (s, 6H), 2.10 (dt, J=13.9, 3.4 Hz, 1H), 1.91 (ddd, J=14.2, 11.0, 3.7 Hz, 1H).

### Example 165: (2R,4R)-6-chloro-N-{3-[1-(4-chlorophenyl)-1H-pyrazol-4-yl]bicyclo[1.1.1]pentan-1-yl}-4-hydroxy-3,4-dihydro-2H-1-benzopyran-2-carboxamide (Compound 264)

### Example 165A: 3-(1-(4-chlorophenyl)-1H-pyrazol-4-yl)bicyclo[1.1.1]pentan-1-amine

The title compound was synthesized using the same procedure as described in Example 163A through Example 163E substituting 6-(trifluoromethyl)pyridin-3-amine with 4-chloroaniline. ¹H NMR (500 MHz, DMSO-d₆) δ ppm 8.60 (br s, 3H), 8.45 (s, 1H), 7.81 (d, J=8.9 Hz, 2H), 7.68 (s, 1H), 7.54 (d, J=8.9 Hz, 2H), 2.24 (s, 6H).

### Example 165B: (2R,4R)-6-chloro-N-{3-[1-(4-chlorophenyl)-1H-pyrazol-4-yl]bicyclo[1.1.1]pentan-1-yl}-4-hydroxy-3,4-dihydro-2H-1-benzopyran-2-carboxamide

The title compound was synthesized using the same procedure as described in Example 163D substituting the product of Example 163C with the product of Example 165A. ¹H NMR (500 MHz, DMSO-d₆) δ ppm 8.70 (s, 1H), 8.39 (s, 1H), 7.83 (d, J=8.9 Hz, 2H), 7.64 (s, 1H), 7.52 (d, J=8.9 Hz, 2H), 7.38 (dd, J=2.7, 0.9 Hz, 1H), 7.20 (dd, J=8.7, 2.7 Hz, 1H), 6.89 (d, J=8.7 Hz, 1H), 5.69 (br s, 1H), 4.81 (dd, J=10.7, 5.9 Hz, 1H), 4.60 (dd, J=12.0, 2.2 Hz, 1H), 2.38-2.34 (m, 1H), 2.28 (s, 6H), 1.74-1.67 (m, 1H).

### Example 166: (2S,4R)-6-chloro-N-{3-[1-(4-chlorophenyl)-1H-pyrazol-4-yl]bicyclo[1.1.1]pentan-1-yl}-4-hydroxy-3,4-dihydro-2H-1-benzopyran-2-carboxamide (Compound 265)

The title compound was synthesized using the same procedure as described in Example 163D substituting the product of Example 163C with the product of Example 165A and the product of Example 3B with the product of Example 73B. ¹H NMR (500 MHz, DMSO-d₆) δ ppm 8.75 (s, 1H), 8.39 (s, 1H), 7.83 (d, J=8.9 Hz, 2H), 7.63 (s, 1H), 7.52 (d, J=8.9 Hz, 2H), 7.31 (d, J=2.7 Hz, 1H), 7.25 (dd, J=8.7, 2.7 Hz, 1H), 6.93 (d, J=8.7 Hz, 1H), 5.61 (d, J=4.5 Hz, 1H), 4.60-4.57 (br m, 1H), 4.55 (dd, J=10.9, 2.8 Hz, 1H), 2.28 (s, 6H), 2.10 (dt, J=13.9, 3.3 Hz, 1H), 1.91 (ddd, J=14.2, 11.0, 3.7 Hz, 1H).

### Example 167: (2R,4R)-6-chloro-4-hydroxy-N-(3-{3-[cis-3-(trifluoromethoxy)cyclobutyl]-1,2-oxazol-5-yl}bicyclo[1.1.1]pentan-1-yl)-3,4-dihydro-2H-1-benzopyran-2-carboxamide (Compound 266)

### Example 167A: cis-N-methoxy-N-methyl-3-(trifluoromethoxy)cyclobutanecarboxamide

To a stirred solution of the product of Example 25N (1.00 g, 5.43 mmol), N,N-diisopropylethylamine (3.78 mL, 21.73 mmol), and N,O-dimethylhydroxylamine hydrochloride (0.636 g, 6.52 mmol) in N,N-dimethylformamide (20 mL), at 0° C. under an atmosphere of nitrogen, was added HATU (1-((dimethylaminodimethyliminio)methyl)-1H-[1,2,3]triazolo[4,5-b]pyridine 3-oxide hexafluorophosphate) (3.10 g, 8.15 mmol) and the reaction mixture was stirred at this temperature for 1 hour, then warmed to ambient temperature and stirred for 18 hours. The reaction mixture was diluted with ethyl acetate (50 mL) and washed with saturated NaHCO₃(aqueous) (25 mL) followed by 1 M HCl (aqueous) (25 mL) then 1:1 brine:water (3×50 mL). The organic phase was dried over Na₂SO₄, filtered, and concentrated in vacuo. The residue was purified by chromatography on silica gel (12 g cartridge, dichloromethane loading, 0-100% ethyl acetate/isohexane) to afford the title compound. This material was further purified by chromatography on silica gel (12 g cartridge, dichloromethane loading, 0-50% ethyl acetate/isohexane) to afford the title compound (976 mg, 3.87 mmol, 71.2% yield). ¹H NMR (500 MHz, CDCl₃) δ ppm 4.61 (p, J=7.6 Hz, 1H), 3.67 (s, 3H), 3.20 (s, 3H), 3.12-2.99 (m, 1H), 2.60-2.52 (m, 4H).

### Example 167B: cis-3-(trifluoromethoxy)cyclobutanecarbaldehyde

The product of Example 167A (978 mg, 4.30 mmol) was dissolved in anhydrous tetrahydrofuran (40 mL) under a nitrogen atmosphere. The solution was cooled to −78° C. and diisobutylaluminum hydride (1.0 M in hexanes) (9.47 mL, 9.47 mmol) was slowly added via syringe. The reaction mixture was stirred at −78° C. for 1 hour. Methanol (0.3 mL) was added and the reaction mixture was stirred at −78° C. for 10 minutes. Hydrochloric acid (1 M aqueous, 55 mL) and dichloromethane (55 mL) were added and the dry ice bath was removed. The mixture was stirred vigorously whilst warming to room temperature and stirring was continued for 2.5 hours. The phases were separated and the aqueous phase was extracted with dichloromethane (50 mL×2). The organic phases were combined, filtered through a hydrophobic phase separator and concentrated under reduced pressure (250 mbar at 40° C.) to afford the crude title compound (723 mg, 4.30 mmol, 100% yield) which was used directly in the subsequent step (assumed quantitative).

### Example 167C: 3-(trifluoromethoxy)cyclobutanecarbaldehyde Oxime

The product of Example 167B (0.778 g, 4.63 mmol) was dissolved in a mixed solvent of ethanol (36 mL) and water (4 mL) at room temperature. Hydroxylamine hydrochloride (1.930 g, 27.8 mmol) and sodium acetate (2.279 g, 27.8 mmol) were added and the reaction mixture was stirred at room temperature for 2 days. The reaction mixture was partitioned between dichloromethane (20 mL) and water (20 mL). The two layers were separated and the aqueous layer was re-extracted with dichloromethane (20 mL). The combined organic layers were passed through a hydrophobic cartridge and concentrated in vacuo to afford the crude title compound (0.848 g, 4.63 mmol, 100% yield).

### Example 167D: cis-N-hydroxy-3-(trifluoromethoxy)cyclobutanecarbimidoyl Chloride

The product of Example 167C (0.133 g, 0.724 mmol) was dissolved in anhydrous N,N-dimethylformamide (1.5 mL). A solution of N-chlorosuccinimide (0.106 g, 0.796 mmol) in anhydrous N,N-dimethylformamide (1 mL) was added slowly to the reaction mixture at 0° C. The reaction mixture was stirred at 0° C. for 1 hour and at room temperature for 4 hours. The reaction mixture was used directly, without analysis, in the subsequent step (assumed quantitative).

### Example 167E: Tert-Butyl (3-(3-(cis-3-(trifluoromethoxy)cyclobutyl)isoxazol-5-yl)bicyclo[1.1.1]pentan-1-yl)carbamate

To a stirred solution of the product of Example 151A (300 mg, 1.447 mmol) and triethylamine (0.202 mL, 1.447 mmol) in anhydrous N,N-dimethylformamide (3 mL) was added the product of Example 167D (0.362 M in N,N-dimethylformamide) (1.999 mL, 0.724 mmol) and the reaction mixture was heated to 60° C. and stirred for 18 hours. The reaction mixture was diluted with ethyl acetate (20 mL) and washed with 1 M HCl (aqueous) (20 mL) then 1:1 brine:water (3×25 mL). The organic phase was dried over Na₂SO₄, filtered, and concentrated in vacuo. The residue was purified by chromatography on silica gel (4 g cartridge, dichloromethane loading, 0-50% ethyl acetate/isohexane) to afford the title compound (170 mg, 0.438 mmol, 30.2% yield). ¹H NMR (500 MHz, DMSO-d₆) δ ppm 7.69 (s, 1H), 6.43 (s, 1H), 4.84 (p, J=7.5 Hz, 1H), 3.21-3.10 (m, 1H), 2.80-2.69 (m, 2H), 2.40-2.28 (m, 2H), 2.25 (s, 6H), 1.39 (s, 9H).

### Example 167F: 3-(3-(cis-3-(trifluoromethoxy)cyclobutyl)isoxazol-5-yl)bicyclo[1.1.1]pentan-1-amine, Trifluoroacetic Acid

To the product of Example 167E (170 mg, 0.438 mmol) in dichloromethane (5 mL) at room temperature was added trifluoroacetic acid (0.506 mL, 6.57 mmol). The reaction mixture was stirred at room temperature for 5 hours. The volatiles were removed under vacuum and co-evaporated with toluene (3×20 mL) to afford the title compound which was used without further purification. MS (ESI⁺) m/z 289.3 (M+H)⁺.

### Example 167G: (2R,4R)-6-chloro-4-hydroxy-N-(3-{3-[cis-3-(trifluoromethoxy)cyclobutyl]-1,2-oxazol-5-yl}bicyclo[1.1.1]pentan-1-yl)-3,4-dihydro-2H-1-benzopyran-2-carboxamide

The product of Example 3B (20 mg, 0.087 mmol) and the product of Example 167F (90 mg, 0.224 mmol) were dissolved in anhydrous N,N-dimethylformamide (1 mL) at room temperature. N,N-Diisopropylethylamine (0.107 mL, 0.612 mmol) and 2,4,6-tripropyl-1,3,5,2,4,6-trioxatriphosphinane 2,4,6-trioxide in N,N-dimethylformamide (50%) (0.061 mL, 0.105 mmol) were added and the reaction mixture was stirred at room temperature for 16 hours. The reaction mixture was purified by preparative HPLC (Waters XBridge™ Prep-C18, 5 μm column (19 mm×50 mm). A 40-70% gradient of acetonitrile (A) and 0.1% ammonium bicarbonate in water (B) was used over 7.5 minutes at a flow rate of 30 mL/minute) to afford the title compound (24 mg, 0.047 mmol, 53.9% yield). ¹H NMR (500 MHz, DMSO-d₆) δ ppm 8.82 (s, 1H), 7.38 (d, J=2.7 Hz, 1H), 7.21 (dd, J=8.7, 2.7 Hz, 1H), 6.89 (d, J=8.7 Hz, 1H), 6.47 (s, 1H), 5.71 (d, J=6.2 Hz, 1H), 4.89-4.78 (m, 2H), 4.62 (dd, 0.1=12.0, 2.3 Hz, 1H), 3.21-3.13 (m, 1H), 2.78-2.70 (m, 2H), 2.40 (s, 6H), 2.37-2.29 (m, 3H), 1.74-1.66 (m, 1H); ¹⁹F NMR (471 MHz, DMSO-d₆) δ ppm −57.77.

### Example 168: (2R,4R)-6-chloro-4-hydroxy-N-(3-{2-oxo-5-[cis-3-(trifluoromethoxy)cyclobutyl]-1,3-oxazolidin-3-yl}bicyclo[1.1.1]pentan-1-yl)-3,4-dihydro-2H-1-benzopyran-2-carboxamide (Compound 267)

### Example 168A: tert-butyl(cis-3-(oxiran-2-yl)cyclobutoxy)diphenylsilane

To a stirred suspension of the product of Example 128G (289 mg, 0.859 mmol) and sodium bicarbonate (72.1 mg, 0.859 mmol) in anhydrous dichloromethane at 0° C. under an atmosphere of nitrogen, was added 3-chlorobenzoperoxoic acid (183 mg, 0.816 mmol) dropwise as a solution in anhydrous dichloromethane (5 mL) and the reaction mixture stirred at this temperature for 1 hour then warmed to room temperature and stirred for 18 hours. The reaction mixture was partitioned between dichloromethane (50 mL) and saturated aqueous sodium hydrogen carbonate (50 mL) The two layers were separated and the aqueous layer was re-extracted with dichloromethane (50 mL). The combined organic layers were passed through a hydrophobic cartridge and concentrated in vacuo to afford a crude colorless solid.

The crude product was purified by chromatography on silica gel (4 g cartridge, dichloromethane loading, 0-10% tert-butyl methyl ether/isohexane) to afford the title compound (191 mg, 0.515 mmol, 59.9% yield) as a colorless solid. ¹H NMR (500 MHz, DMSO-d₆) δ ppm 7.59 (ddq, J=7.2, 3.1, 1.4 Hz, 4H), 7.47-7.40 (m, 6H), 4.12-4.06 (m, 1H), 2.92 (td, J=3.9, 2.6 Hz, 1H), 2.64 (dd, J=5.0, 4.0 Hz, 1H), 2.36 (dd, J=5.0, 2.7 Hz, 1H), 2.22-2.14 (m, 1H), 2.08-1.97 (m, 1H), 1.85-1.74 (m, 2H), 1.72-1.63 (m, 1H), 0.97 (s, 9H).

### Example 168B: Tert-Butyl (3-((2-((cis)-3-((tert-butyldiphenylsilyl)oxy)cyclobutyl)-2-hydroxyethyl)amino)bicyclo[1.1.1]pentan-1-yl)carbamate

A mixture of tert-butyl (3-aminobicyclo[1.1.1]pentan-1-yl)carbamate (0.215 g, 1.084 mmol) and Example 168A (0.191 g, 0.542 mmol) were combined in ethanol (3 mL) and the reaction mixture stirred at 60° C., under an atmosphere for nitrogen, for 18 hours. The reaction mixture was concentrated in vacuo and the crude oil was purified by chromatography on silica gel (4 g cartridge, dichloromethane loading, 0-100% ethyl acetate/isohexane, then 10% methanol/dichloromethane) to afford the title compound (0.257 g, 0.466 mmol, 86% yield) that was used without additional purification. MS (ESI⁺) m/z 551.3 (M+H)⁺.

### Example 168: Tert-Butyl (3-(5-(cis-3-((tert-butyldiphenylsilyl)oxy)cyclobutyl)-2-oxooxazolidin-3-yl)bicyclo[1.1.1]pentan-1-yl)carbamate

To a stirred solution of the product of Example 168B (133. mg, 0.241 mmol) in tetrahydrofuran (1 mL), at room temperature under an atmosphere of nitrogen, was added 1,1′-carbonyldiimidazole (86 mg, 0.531 mmol) and the reaction mixture was stirred for 3 hours. After removal of solvent, the residue was purified by chromatography on silica gel (4 g cartridge, dichloromethane loading, 0-100% ethyl acetate/isohexane) to afford the title compound (103 mg, 0.167 mmol, 69.3% yield). MS (ESI⁺) m/z 599.2 (M+Na)⁺.

### Example 168D: Tert-Butyl (3-(5-(cis-3-hydroxycyclobutyl)-2-oxooxazolidin-3-yl)bicyclo[1.1.1]pentan-1-yl)carbamate

To a solution of the product of Example 168C (119 mg, 0.206 mmol) in tetrahydrofuran (2. mL), at 0° C. under an atmosphere of nitrogen, was added tetrabutylammonium fluoride (0.413 mL, 0.413 mmol) (1 M in tetrahydrofuran) and the reaction mixture warmed to room temperature and stirred for 22 hours. Additional tetrabutylammonium fluoride (0.206 mL, 0.206 mmol) was added and the reaction mixture was stirred for an additional 3 hours. The reaction mixture was quenched with saturated NH₄Cl (aqueous) (10 mL) and extracted with dichloromethane (2×10 mL). The combined organic layers were passed through a hydrophobic phase separator and concentrated in vacuo. The residue was purified by chromatography on silica gel (4 g cartridge, dichloromethane loading, 0-100% ethyl acetate/isohexane followed by 10% methanol/dichloromethane) to afford the title compound (127 mg, 0.270 mmol, 131% yield). MS (ESI⁺) m/z 339.1 (M+H)⁺.

### Example 168E: Tert-Butyl (3-(2-oxo-5-(cis-3-(trifluoromethoxy)cyclobutyl)oxazolidin-3-yl)bicyclo[1.1.1]pentan-1-yl)carbamate

The title compound was synthesized using the same procedure as described in Example 13O substituting the product of Example 13N with the product of Example 168D. ¹⁹F NMR (471 MHz, DMSO-d₆) δ ppm −57.68.

### Example 168F: 3-(3-aminobicyclo[1.1.1]pentan-1-yl)-5-(cis-3-(trifluoromethoxy)cyclobutyl)oxazolidin-2-one Trifluoroacetic Acid

To the product of Example 168E (123 mg, 0.303 mmol) in dichloromethane (5 mL) at room temperature was added trifluoroacetic acid (0.350 mL, 4.54 mmol). The reaction mixture was stirred at room temperature for 3 hours. The volatiles were removed under vacuum and co-evaporated with toluene (3×20 mL) to afford the title compound (126 mg, 0.270 mmol, 89% yield). The product was carried forward for the next step without further purification.

### Example 168G: (2R,4R)-6-chloro-4-hydroxy-N-(3-{2-oxo-5-[cis-3-(trifluoromethoxy)cyclobutyl]-1,3-oxazolidin-3-yl}bicyclo[1.1.1]pentan-1-yl)-3,4-dihydro-2H-1-benzopyran-2-carboxamide

To a mixture of the product of Example 3B (20 mg, 0.087 mmol) and the product of Example 168F (60 mg, 0.143 mmol) in N,N-dimethylformamide (1 mL) at room temperature, N,N-diisopropylethylamine (0.107 mL, 0.612 mmol) and 2,4,6-tripropyl-1,3,5,2,4,6-trioxatriphosphinane 2,4,6-trioxide in N,N-dimethylformamide (50%) (0.061 mL, 0.105 mmol) were added and the reaction mixture was stirred at room temperature for 16 hours. The reaction mixture was purified by preparative HPLC (Waters X-Bridge™ Prep-C18, 5 μm column (19 mm×50 mm). A 40-70% gradient of acetonitrile (A) and 0.1% ammonium bicarbonate in water (B) was used over 7.5 minutes at a flow rate of 30 mL/minute) to afford the title compound (12 mg, 0.023 mmol, 26.0% yield). ¹H NMR (500 MHz, DMSO-d₆) δ ppm 8.74 (s, 1H), 7.38 (d, J=2.7 Hz, 1H), 7.21 (dd, J=8.7, 2.7 Hz, 1H), 6.89 (d, J=8.7 Hz, 1H), 5.70 (d, J=6.3 Hz, 1H), 4.84-4.78 (m, 1H), 4.77-4.71 (m, 1H), 4.61 (dd, J=12.0, 2.3 Hz, 1H), 4.53 (q, J=6.6 Hz, 1H), 3.61 (t, J=8.7 Hz, 1H), 3.10 (dd, J=8.9, 6.7 Hz, 1H), 2.46-2.30 (m, 2H), 2.28 (s, 6H), 2.26-2.11 (m, 2H), 2.05-1.94 (m, 2H), 1.70 (q, J=11.9 Hz, 1H); ¹⁹F NMR (471 MHz, DMSO-d₆) δ ppm −57.68.

### Example 169: (2S,4R)-6-chloro-4-hydroxy-N-(3-{3-[cis-3-(trifluoromethoxy)cyclobutyl]-1,2-oxazol-5-yl}bicyclo[1.1.1]pentan-1-yl)-3,4-dihydro-2H-1-benzopyran-2-carboxamide (Compound 268)

The title compound was synthesized using the same procedure as described in Example 167G substituting the product of Example 3B with the product of Example 73B. ¹H NMR (500 MHz, DMSO-d₆) δ ppm 8.88 (s, 1H), 7.32 (d, J=2.7 Hz, 1H), 7.26 (dd, J=8.7, 2.7 Hz, 1H), 6.93 (d, J=8.8 Hz, 1H), 6.47 (s, 1H), 5.62 (d, J=4.7 Hz, 1H), 4.85 (p, J=7.5 Hz, 1H), 4.61-4.54 (m, 2H), 3.22-3.12 (m, 1H), 2.78-2.70 (m, 2H), 2.39 (s, 6H), 2.37-2.28 (m, 2H), 2.13-2.07 (m, 1H), 1.95-1.87 (m, 1H); ¹⁹F NMR (471 MHz, DMSO-d₆) δ ppm −57.77.

### Example 170: (2S,4R)-6-chloro-4-hydroxy-N-(3-{2-oxo-5-[cis-3-(trifluoromethoxy)cyclobutyl]-1,3-oxazolidin-3-yl}bicyclo[1.1.1]pentan-1-yl)-3,4-dihydro-2H-1-benzopyran-2-carboxamide (Compound 269)

The title compound was synthesized using the same procedure as described in Example 168G substituting the product of Example 3B with the product of Example 73B. ¹H NMR (500 MHz, DMSO-d₆) δ ppm 8.80 (s, 1H), 7.32 (d, J=2.6 Hz, 1H), 7.26 (dd, J=8.7, 2.7 Hz, 1H), 6.94 (d, J=8.8 Hz, 1H), 5.61 (d, J=4.7 Hz, 1H), 4.74 (p, J=7.6 Hz, 1H), 4.60-4.50 (m, 3H), 3.61 (t, J=8.7 Hz, 1H), 3.10 (dd, J=8.9, 6.7 Hz, 1H), 2.47-2.36 (m, 1H), 2.28 (s, 6H), 2.24-2.15 (m, 1H), 2.13-1.86 (m, 5H); ¹⁹F NMR (471 MHz, DMSO-d₆) δ ppm −57.68.

### Example 171: (2R,4R)-6-chloro-N-{3-[5-(4-chloro-3-fluorophenyl)-1,3-oxazol-2-yl]bicyclo[1.1.1]pentan-1-yl}-4-hydroxy-3,4-dihydro-2H-1-benzopyran-2-carboxamide (Compound 270)

### Example 171A: 2-bromo-1-(4-chloro-3-fluorophenyl)ethanone

To a solution of 1-(4-chloro-3-fluorophenyl)ethanone (1.00 g, 5.79 mmol) in dichloromethane (10 mL) and methanol (30 mL) was added tetrabutylammonium tribromide (2.79 g, 5.79 mmol) portionwise. The resulting solution was stirred at ambient temperature overnight. The reaction mixture was then concentrated under reduced pressure. The resulting residue was then dissolved in ethyl acetate (20 mL) and washed with water (3×20 mL). The organic layer was concentrated under reduced pressure to give the title intermediate (1.30 g, 4.65 mmol, 80% yield). ¹H NMR (500 MHz, DMSO-d₆) δ ppm 8.01 (dd, J=10.0, 2.0 Hz, 1H), 7.87-7.80 (m, 2H), 4.96 (s, 2H).

### Example 171B: 2-amino-1-(4-chloro-3-fluorophenyl)ethanone Hydrochloride

The methodologies described in Example 193D substituting Example 171A for Example 193C gave the title intermediate. ¹H NMR (500 MHz, DMSO-d₆) δ ppm 8.39 (s, 3H), 8.07 (dd, J=9.9, 1.8 Hz, 1H), 7.91-7.85 (m, 2H), 4.61 (s, 2H).

### Example 171C: Tert-Butyl (3-((2-(4-chloro-3-fluorophenyl)-2-oxoethyl)carbamoyl)bicyclo[1.1.1]pentan-1-yl)carbamate

The methodologies described in Example 193E substituting 3-((tert-butoxycarbonyl)amino)bicyclo[1.1.1]pentane-1-carboxylic acid (PharmaBlock) for (2S,5R)-5-((tert-butoxycarbonyl)amino)tetrahydro-2H-pyran-2-carboxylic acid, substituting Example 171B for 193D, and including a trituration of the crude intermediate, washing with tert-butyl methyl ether (3×10 mL) gave the title intermediate. ¹H NMR (500 MHz, DMSO-d₆) δ ppm 8.14 (t, J=5.7 Hz, 1H), 7.98 (dd, J=9.9, 1.8 Hz, 1H), 7.83-7.80 (m, 1H), 7.21 (s, 1H), 6.91 (s, 1H), 4.52 (d, J=5.7 Hz, 2H), 2.01 (s, 6H), 1.37 (s, 9H); MS (ESI⁺) m/z 397 (M+H)⁺.

### Example 171D: 3-(5-(4-chloro-3-fluorophenyl)oxazol-2-yl)bicyclo[1.1.1]pentan-1-amine

The methodologies described in Example 155B substituting Example 171C for Example 155A gave the title intermediate. ¹H NMR (500 MHz, DMSO-d₆) δ ppm 7.75 (dd, J=10.4, 2.0 Hz, 1H), 7.71-7.66 (m, 2H), 7.53 (dd, J=8.4, 2.0 Hz, 1H), 2.13 (s, 6H); MS (ESI⁺) m/z 262 (M−NH₂+H)⁺.

### Example 171E: (2R,4R)-6-chloro-N-{3-[5-(4-chlorophenyl)-1,3-oxazol-2-yl]bicyclo[1.1.1]pentan-1-yl}-4-hydroxy-3,4-dihydro-2H-1-benzopyran-2-carboxamide

The methodologies described in Example 155C substituting Example 171D for Example 155B gave the title compound. ¹H NMR (500 MHz, DMSO-d₆) δ ppm 8.87 (s, 1H), 7.78 (dd, J=10.4, 2.0 Hz, 1H), 7.75 (s, 1H), 7.70 (t, J=8.1 Hz, 1H), 7.57 (dd, J=8.6, 1.9 Hz, 1H), 7.40 (dd, J=2.7, 1.0 Hz, 1H), 7.22 (dd, J=8.7, 2.7 Hz, 1H), 6.90 (d, J=8.7 Hz, 1H), 5.72 (d, J=6.3 Hz, 1H), 4.85-4.81 (m, 1H), 4.64 (dd, J=12.0, 2.3 Hz, 1H), 2.50 (s, 6H), 2.42-2.37 (m, 1H), 1.72 (q, J=11.7 Hz, 1H); MS (ESI⁺) m/z 489/491 (³⁵Cl/³⁷Cl, M+H)⁺.

### Example 172: (2S,4R)-6-chloro-N-{3-[5-(4-chloro-3-fluorophenyl)-1,3-oxazol-2-yl]bicyclo[1.1.1]pentan-1-yl}-4-hydroxy-3,4-dihydro-2H-1-benzopyran-2-carboxamide (Compound 271)

The methodologies described in Example 155C substituting the product of Example 73B for Example 3B and 171D for Example 155B gave the title compound. ¹H NMR (500 MHz, DMSO-d₆) δ ppm 8.93 (s, 1H), 7.78 (dd, J=10.3, 2.0 Hz, 1H), 7.75 (s, 1H), 7.70 (t, J=8.1 Hz, 1H), 7.56 (dd, J=8.2, 2.0 Hz, 1H), 7.33 (d, J=2.7 Hz, 1H), 7.27 (dd, J=8.7, 2.7 Hz, 1H), 6.95 (d, J=8.7 Hz, 1H), 5.64 (d, J=4.7 Hz, 1H), 4.63-4.56 (m, 2H), 2.49 (s, 6H), 2.14-2.10 (m, 1H), 1.95-1.90 (m, 1H); MS (ESI⁺) m/z 489/491 (³⁵Cl/³⁷Cl, M+H)⁺.

### Example 173: (2R,4R)-6-chloro-N-{3-[2-(4-chlorophenyl)-1,3-thiazol-4-yl]bicyclo[1.1.1]pentan-1-yl}-4-hydroxy-3,4-dihydro-2H-1-benzopyran-2-carboxamide (Compound 272)

### Example 173A: 3-(2-(4-chlorophenyl)thiazol-4-yl)bicyclo[1.1.1]pentan-1-amine

To a solution of the product of Example 181B (50 mg, 0.164 mmol) in ethanol (2 mL) was added 4-chlorobenzothioamide (31.0 mg, 0.181 mmol). The resulting solution was stirred at 80° C. for 2 hours. The reaction mixture was then concentrated under reduced pressure. To the crude mixture, dichloromethane (4 mL) and trifluoroacetic acid (0.307 mL, 3.98 mmol) were added. The resulting solution was stirred at ambient temperature for 2 hours. To the reaction mixture was added SCX resin (1 g) and the suspension was stirred for 30 minutes, the SCX was collected by filtration and washed with methanol (20 mL). The product was then eluted with NH₃ in MeOH (3.5 M) and the filtrate was concentrated in vacuo to afford the title compound (80 mg, 100% yield). ¹H NMR (500 MHz, DMSO-d₆) δ ppm 7.96-7.89 (m, 2H), 7.59-7.52 (m, 2H), 7.38 (s, 1H), 2.31 (s, 2H), 2.03 (s, 6H); MS (ESI) m/z 277 (M+H)⁺.

### Example 173B: (2R,4R)-6-chloro-N-{3-[2-(4-chlorophenyl)-1,3-thiazol-4-yl]bicyclo[1.1.1]pentan-1-yl}-4-hydroxy-3,4-dihydro-2H-1-benzopyran-2-carboxamide

The title compound was prepared using the method described for the synthesis of Example 141E, substituting the product from Example 141D with the product of Example 173A. ¹H NMR (500 MHz, DMSO-d₆) δ ppm 8.77 (s, 1H), 7.98-7.91 (m, 2H), 7.60-7.53 (m, 2H), 7.50 (s, 1H), 7.42-7.38 (m, 1H), 7.25-7.19 (m, 1H), 6.91 (d, J=8.7 Hz, 1H), 5.72 (d, J=6.2 Hz, 1H), 4.87-4.79 (m, 1H), 4.63 (dd, J=12.0, 2.3 Hz, 1H), 2.42-2.32 (m, 7H), 1.78-1.68 (m, 1H); MS (ESI) m/z 487 (M+H)⁺.

### Example 174: (2S,4R)-6-chloro-N-{3-[2-(4-chlorophenyl)-1,3-thiazol-4-yl]bicyclo[11.11]pentan-1-yl}-4-hydroxy-3,4-dihydro-2H-1-benzopyran-2-carboxamide (Compound 273)

The title compound was prepared using the methods described for the synthesis of Example 141E, substituting the product of Example 141D with the product from Example 173A, and substituting the product of Example 3B with the product of example 73B. ¹H NMR (500 MHz, DMSO-d₆) δ ppm 8.83 (s, 1H), 7.99-7.89 (m, 2H), 7.61-7.53 (m, 2H), 7.50 (s, 1H), 7.33 (d, J=2.7 Hz, 1H), 7.27 (dd, J=8.7, 2.7 Hz, 1H), 6.96 (d, J=8.7 Hz, 1H), 5.63 (d, J=4.7 Hz, 1H), 4.64-4.55 (m, 2H), 2.38 (s, 6H), 2.16-2.10 (m, 1H), 1.98-1.89 (m, 1H); MS (ESI) m/z 487 (M+H)⁺.

### Example 175: (2R,4R)-6-chloro-N-{3-[5-(4-chlorophenyl)-4-methyl-1,3-oxazol-2-yl]bicyclo[1.1.1]pentan-1-yl}-4-hydroxy-3,4-dihydro-2H-1-benzopyran-2-carboxamide (Compound 274)

### Example 175A: 2-amino-1-(4-chlorophenyl)propan-1-one Hydrochloride

The methodologies described in Example 193D substituting 2-bromo-1-(4-chlorophenyl)propan-1-one (Apollo) for Example 193C and modifying each of the reaction times to 16 hours gave the title intermediate. ¹H NMR (500 MHz, DMSO-d₆) δ ppm 8.13-8.06 (m, 2H), 7.71-7.67 (m, 2H), 7.40 (br, s, 3H), 5.11 (q, J=7.2 Hz, 1H), 1.41 (d, J=7.2 Hz, 3H); MS (ESI+) m/z 184 (M+H)⁺.

### Example 175B: Tert-Butyl (3-((1-(4-chlorophenyl)-1-oxopropan-2-yl)carbamoyl)bicyclo[1.1.1]pentan-1-yl)carbamate

The methodologies described in Example 155A substituting Example 175A for 2-amino-1-(4-chlorophenyl)ethanone hydrochloride gave the title intermediate. ¹H NMR (500 MHz, DMSO-d₆) δ ppm 7.95-7.91 (m, 2H), 7.61-7.57 (m, 2H), 7.20 (s, 1H), 6.91 (s, 1H), 5.19-5.17 (m, 1H), 2.01 (s, 6H), 1.37 (s, 9H), 1.26 (d, J=7.1 Hz, 3H); MS (ESI⁺) m/z 393 (M+H)⁺.

### Example 175C: 3-(5-(4-chlorophenyl)-4-methyloxazol-2-yl)bicyclo[1.1.1]pentan-1-amine

The methodologies described in Example 155B substituting Example 175B for Example 155A gave the title intermediate. ¹H NMR (500 MHz, DMSO-d₆) δ ppm 7.60-7.56 (m, 2H), 7.55-7.50 (m, 2H), 2.29 (s, 3H), 2.09 (s, 6H); MS (ESI⁺) m/z 275 (M+H)⁺.

### Example 175D: (2R,4R)-1-chloro-N-{3-[S-(4-chlorophenyl)-4-methyl-,3-oxazol-2-yl]bicyclo[1.1.1]pentan-1-yl}-4-hydroxy-3,4-dihydro-2H-1-benzopyran-2-carboxamide

The methodologies described in Example 155C substituting Example 175C for Example 155B gave the title compound. ¹H NMR (500 MHz, DMSO-d₆) δ ppm 8.86 (s, 1H), 7.63-7.59 (m, 2H), 7.57-7.53 (m, 2H), 7.39 (dd, J=2.8, 1.0 Hz, 1H), 7.22 (dd, J=8.7, 2.7 Hz, 1H), 6.90 (d, J=8.7 Hz, 1H), 5.72 (d, J=6.3 Hz, 1H), 4.84-4.81 (m, 1H), 4.63 (dd, J=12.0, 2.3 Hz, 1H), 2.47 (s, 6H), 2.42-2.38 (m, 1H), 2.32 (s, 3H), 2.09 (d, J=5.9 Hz, 1H); MS (ESI⁺) m/z 486 (M+H)⁺.

### Example 176: (2S,4R)-6-chloro-N-{3-[5-(4-chlorophenyl)-4-methyl-1,3-oxazol-2-yl]bicyclo[1.1.1]pentan-1-yl}-4-hydroxy-3,4-dihydro-2H-1-benzopyran-2-carboxamide (Compound 275)

The methodologies described in Example 155C substituting Example 175C for Example 155B and substituting the product of Example 73B for Example 3B gave the title compound. ¹H NMR (500 MHz, DMSO-d₆) δ ppm 8.91 (s, 1H), 7.64-7.58 (m, 2H), 7.57-7.52 (m, 2H), 7.33 (d, J=2.7 Hz, 1H), 7.27 (dd, J=8.7, 2.7 Hz, 1H), 6.95 (d, J=8.8 Hz, 1H), 5.63 (d, J=4.6 Hz, 1H), 4.61-4.56 (m, 2H), 2.46 (s, 6H), 2.32 (s, 3H), 2.14-2.10 (m, 1H), 1.94-1.90 (m, 1H); MS (ESI+) m/z 486 (M+H)⁺.

### Example 177: (2S,4S)-6-chloro-4-hydroxy-N-[(3S)-3-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 276)

### Example 177A: Tert-Butyl [(2S)-4-{[(2S)-6-chloro-4-oxo-3,4-dihydro-2H-1-benzopyran-2-carbonyl]amino}-2-hydroxybicyclo[2.2.2]octan-1-yl]carbamate

The reaction and purification conditions described in Example 2B substituting the product of Example 13H for the product of Example 2A, and the product of Example 10A for the product of Example 1B gave the title compound. MS (APCI⁺) m/z 465 (M+H)⁺.

### Example 177B: (2S)—N-[(3S)-4-amino-3-hydroxybicyclo[2.2.2]octan-1-yl]-6-chloro-4-oxo-3,4-dihydro-2H-1-benzopyran-2-carboxamide

Trifluoroacetic acid (1 mL) was added to a solution the product of Example 177A (0.28 g, 0.60 mmol) in dichloromethane (2 mL) and the resulting mixture was stirred at ambient temperature for 1 hour and then concentrated under reduced pressure. The residue was taken up in methanol (5 mL) and directly purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (0.2 g, 0.55 mmol, 91% yield). MS (APCI⁺) m/z 365 (M+H)⁺.

### Example 177: (2S,4S)-6-chloro-4-hydroxy-N-[(3S)-3-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide

The reaction and purification conditions described in Example 136D substituting the product of Example 177B for the product of Example 136C, and the product of Example 13P for the product of Example 1B gave the title compound. ¹H NMR (400 MHz, DMSO-d₆) δ ppm 7.37 (dd, J=2.8, 1.0 Hz, 1H), 7.34 (d, J=1.7 Hz, 1H), 7.17 (dd, J=8.6, 2.7 Hz, 1H), 6.93 (s, 1H), 6.86 (d, J=8.7 Hz, 1H), 5.66 (s, 1H), 5.21 (s, 1H), 4.77 (dd, J=10.4, 6.0 Hz, 1H), 4.54 (dd, J=11.7, 2.2 Hz, 1H), 4.48 (p, J=7.1 Hz, 1H), 3.94 (d, J=9.1 Hz, 1H), 3.78-3.65 (m, 3H), 2.80-2.69 (m, 2H), 2.35-2.18 (m, 3H), 2.17-2.06 (m, 2H), 2.02-1.66 (m, 9H); MS (APCI⁺) m/z 563 (M+H)⁺.

### Example 178: (2R,4R)-6-chloro-N-{3-[5-(4-chlorophenyl)-2-oxo-1,3-oxazolidin-3-yl]bicyclo[1.1.1]pentan-1-yl}-4-hydroxy-3,4-dihydro-2H-1-benzopyran-2-carboxamide (Compound 277)

The title compound was synthesized using the same procedures as described in Example 168A through Example 168C and Example F through Example G substituting Example 128G with 1-chloro-4-vinylbenzene. ¹H NMR (500 MHz, DMSO-d₆) δ ppm 8.75 (s, 1H), 7.52-7.48 (m, 2H), 7.45-7.41 (m, 2H), 7.39-7.37 (m, 1H), 7.20 (dd, J=8.7, 2.6 Hz, 1H), 6.88 (d, J=8.7 Hz, 1H), 5.69 (d, J=6.0 Hz, 1H), 5.59 (t, J=8.0 Hz, 1H), 4.81 (dd, J=10.8, 5.7 Hz, 1H), 4.61 (dd, J=12.0, 2.2 Hz, 1H), 3.98 (t, J=8.8 Hz, 1H), 3.42 (dd, J=9.0, 7.4 Hz, 1H), 2.38-2.27 (m, 7H), 1.73-1.65 (m, 1H).

### Example 179: (2S,4R)-6-chloro-N-{3-[5-(4-chlorophenyl)-2-oxo-1,3-oxazolidin-3-yl]bicyclo[1.1.1]pentan-1-yl}-4-hydroxy-3,4-dihydro-2H-1-benzopyran-2-carboxamide (Compound 278)

The title compound was synthesized using the same procedure as described in Example 178 substituting Example 3B with Example 73B. ¹H NMR (500 MHz, DMSO-d₆) δ ppm 8.81 (s, 1H), 7.52-7.48 (m, 2H), 7.45-7.42 (m, 2H), 7.31 (d, J=2.7 Hz, 1H), 7.25 (dd, J=8.8, 2.7 Hz, 1H), 6.93 (d, J=8.7 Hz, 1H), 5.63-5.56 (m, 2H), 4.59-4.54 (m, 2H), 3.97 (t, J=8.8 Hz, 1H), 3.42 (dd, J=9.0, 7.3 Hz, 1H), 2.31 (s, 6H), 2.09 (dt, J=14.0, 3.4 Hz, 1H), 1.93-1.86 (m, 1H).

### Example 180: (2S,4R)-6-chloro-4-hydroxy-N-[(3S)-3-hydroxy-4-(2-{[cis-3-(trifluoromethoxy)cyclobutyl]oxy}acetamido)bicyclo[2.2.2]octan-1-yl]-3,4-dihydro-2H-1-benzopyran-2-carboxamide (Compound 279)

The reaction conditions described in Example 99, substituting the product of Example 177C for the product of Example 6C, and purification by chiral preparative HPLC [CHIRALPAK® AD-H 5 μm column, 20×250 mm, flow rate 10 mL/minute, 100% ethanol (isocratic gradient)] gave the title compound as the later eluting fraction. ¹H NMR (400 MHz, DMSO-d₆) δ ppm 7.42 (s, 1H), 7.30 (d, J=2.6 Hz, 1H), 7.22 (dd, J=8.7, 2.7 Hz, 1H), 6.93 (s, 1H), 6.90 (d, J=8.7 Hz, 1H), 5.59 (s, 1H), 5.21 (s, 1H), 4.60-4.50 (m, 2H), 4.47 (p, J=7.1 Hz, 1H), 3.93 (dd, J=9.5, 3.3 Hz, 1H), 3.77-3.65 (m, 3H), 2.80-2.69 (m, 2H), 2.35-2.18 (m, 2H), 2.16-2.06 (m, 2H), 2.04-1.66 (m, 10H); MS (APCI⁺) m/z 563 (M+H)⁺.

### Example 181: (2R,4R)-6-chloro-4-hydroxy-N-(3-{2-[cis-3-(trifluoromethoxy)cyclobutyl]-1,3-thiazol-4-yl}bicyclo[1.1.1]pentan-1-yl)-3,4-dihydro-2H-1-benzopyran-2-carboxamide (Compound 280)

### Example 181A: Tert-Butyl (3-(methoxy(methyl)carbamoyl)bicyclo[1.1.1]pentan-1-yl)carbamate

To an ice-cooled solution of NO-dimethylhydroxylamine, hydrochloric acid (1.931 g, 19.80 mmol) and 3-((tert-butoxycarbonyl)amino)bicyclo[1.1.1]pentane-1-carboxylic acid (3.00 g, 13.20 mmol) in dichloromethane (50 mL) was added N,N-diisopropylethylamine (9.22 mL, 52.8 mmol) followed by 1-[bis(dimethylamino)methylene]-1H-1,2,3-triazolo[4,5-b]pyridinium 3-oxid hexafluorophosphate (HATU, 7.53 g, 19.80 mmol), and the reaction mixture was stirred at room temperature for 1 hour. The reaction mixture was diluted with dichloromethane (75 mL) and washed with 1 M aqueous HCl (100 mL), saturated aqueous NaHCO₃ (2×100 mL) and brine (100 mL). The organic phase was dried over MgSO₄, filtered and concentrated in vacuo. The residue was purified by column chromatography on silica gel using a solvent gradient of 0-50% ethyl acetate in isohexane to afford the title compound (3.18 g, 11.18 mmol, 85% yield). ¹H NMR (500 MHz, DMSO-d₆) δ ppm 7.59 (s, 1H), 3.63 (s, 3H), 3.08 (s, 3H), 2.15 (s, 6H), 1.38 (s, 9H).

### Example 181B: Tert-Butyl (3-(2-bromoacetyl)bicyclo[1.1.1]pentan-1-yl)carbamate

To an ice-cooled solution of the product from Example 181A (3.18 g, 11.76 mmol) in anhydrous tetrahydrofuran (100 mL) was added methylmagnesium bromide (3.0 M in diethyl ether, 11.76 mL, 35.3 mmol) dropwise. The resulting solution was allowed to warm to room temperature and stirred overnight. The reaction mixture was quenched with 1 M aqueous HCl (100 mL) and extracted with dichloromethane (100 mL). The organic layer was collected and the volatiles were removed in vacuo to give tert-butyl (3-acetylbicyclo[1.1.1]pentan-1-yl)carbamate (2.62 g, 10.47 mmol, 89% yield). A portion of the tert-butyl (3-acetylbicyclo[1.1.1]pentan-1-yl)carbamate (1.00 g, 4.44 mmol) was dissolved in tetrahydrofuran (10 mL), and phenyltrimethylammonium tribromide (1.669 g, 4.44 mmol) was added portionwise. The resulting solution was stirred at room temperature for 2 hours. The mixture was filtered, washing with tetrahydrofuran (5 mL), and the filtrate was concentrated in vacuo. The residue was purified by column chromatography on silica gel, eluting with a solvent gradient of 0-50% ethyl acetate in isohexane to afford the title compound (244 mg, 0.762 mmol, 17% yield). ¹H NMR (500 MHz, DMSO-d₆) δ ppm 7.63 (s, 1H), 4.46 (s, 2H), 2.19 (s, 6H), 1.38 (s, 9H).

### Example 181C: cis-3-(trifluoromethoxy)cyclobutanecarbothioamide

To a solution of the product from Example 25N (600 mg, 3.26 mmol) in dichloromethane (5 mL) was added ammonium chloride (1.74 g, 32.6 mmol), N,N-diisopropylethylamine (7.40 mL, 42.4 mmol) and 2-(3H-[1,2,3]triazolo[4,5-b]pyridin-3-yl)-1,1,3,3-tetramethylisouronium hexafluorophosphate(V) (1.24 g, 3.26 mmol), and the resulting mixture was stirred at ambient temperature overnight. The mixture was partitioned between dichloromethane (30 mL) and 1 M aqueous HCl solution (30 mL), the aqueous layer was extracted with dichloromethane (30 mL), and the combined organic layers were passed through a hydrophobic cartridge and concentrated in vacuo. The residue was purified by column chromatography on silica gel, eluting with a solvent gradient of 0-100% ethyl acetate in isohexane to afford cis-3-(trifluoromethoxy)cyclobutanecarboxamide (691 mg, 1.170 mmol, 35.9% yield). To a solution of cis-3-(trifluoromethoxy)cyclobutanecarboxamide (691 mg, 3.77 mmol) in tetrahydrofuran (20 mL) was added Lawesson's Reagent (2,4-bis(4-methoxyphenyl)-1,3-dithia-2,4-diphosphetane-2,4-disulfide) (916 mg, 2.264 mmol), and the resulting mixture was stirred at room temperature overnight. The mixture was concentrated onto silica, and the crude product was purified by column chromatography on silica gel, eluting with a solvent gradient of 0-100% ethyl acetate in isohexane to afford the title compound (125 mg, 0.615 mmol, 16.3% yield). ¹H NMR (500 MHz, DMSO-d₆) δ ppm 9.52 (s, 1H), 9.20 (s, 1H), 4.77 (p, J=7.5 Hz, 1H), 2.99-2.89 (m, 1H), 2.61-2.52 (m, 2H), 2.49-2.41 (m, 2H).

### Example 181D: Tert-Butyl (3-(2-(cis-3-(trifluoromethoxy)cyclobutyl)thiazol-4-yl)bicyclo[1.1.1]pentan-1-yl)carbamate

To a solution of the product from Example 181B (100 mg, 0.329 mmol) in ethanol (2 mL) was added the product from Example 181C (65.5 mg, 0.329 mmol). The resulting solution was stirred at 80° C. for 1 hour and concentrated in vacuo to afford the title compound. MS (ESI) m/z 405 (M+H)⁺.

### Example 181E: (2R,4R)-6-chloro-4-hydroxy-N-(3-{2-[cis-3-(trifluoromethoxy)cyclobutyl]-1,3-thiazol-4-yl}bicyclo[1.1.1]pentan-1-yl)-3,4-dihydro-2H-1-benzopyran-2-carboxamide

The title compound was prepared using the methods described for the synthesis of Example 131D, substituting the product from Example 181D for the product from Example 131C, and substituting the product from Example 3B for the product from Example 73B. ¹H NMR (500 MHz, DMSO-d₆) δ ppm 8.73 (s, 1H), 7.38 (dd, J=3.0, 1.0 Hz, 1H), 7.29 (s, 1H), 7.20 (dd, J=8.5, 2.5 Hz, 1H), 6.90 (d, J=8.5 Hz, 1H), 5.70 (d, J=6.5 Hz, 1H), 4.89-4.77 (m, 2H), 4.61 (dd, J=12.0, 2.5 Hz, 1H), 3.51-3.42 (m, 1H), 2.91-2.80 (m, 2H), 2.44-2.33 (m, 3H), 2.32 (s, 6H), 1.78-1.64 (m, 1H); MS (ESI) m/z 515 (M+H)⁺.

### Example 182: (2R,4R)-6-chloro-N-{3-[4-(4-chlorophenyl)-1H-imidazol-1-yl]bicyclo[1.1.1]pentan-1-yl}-4-hydroxy-3,4-dihydro-2H-1-benzopyran-2-carboxamide (Compound 281)

### Example 182A: Tert-Butyl (3-(4-(4-chlorophenyl)-1H-imidazol-1-yl)bicyclo[1.1.1]pentan-1-yl)carbamate

The methodologies described in Example 49A substituting 4-chlorobenzaldehyde for 3,4-difluorobenzaldehyde and purifying by column chromatography on silica gel (0-100% ethyl acetate in isohexane) gave the title intermediate. ¹H NMR (500 MHz, DMSO-d₆) δ ppm 7.79-7.76 (m, 3H), 7.74 (d, J=1.3 Hz, 1H), 7.42-7.39 (m, 2H), 2.40 (s, 6H), 1.41 (s, 9H); MS (ESI+) m/z 360 (M+H)⁺.

### Example 182B: 3-(4-(4-chlorophenyl)-1H-imidazol-1-yl)bicyclo[1.1.1]pentan-1-amine, Trifluoroacetic Acid

The methodologies described in Example 21B substituting Example 182A for Example 21A gave the title intermediate as a trifluoroacetic acid salt. ¹H NMR (500 MHz, DMSO-d₆) δ ppm 8.91 (s, 2H), 8.58 (s, 1H), 8.14 (s, 1H), 7.82-7.75 (m, 2H), 7.56-7.51 (m, 2H), 2.58 (s, 6H); MS (ESI+) m/z 260 (M+H)⁺.

### Example 182C: (2R,4R)-6-chloro-N-{3-[4-(4-chlorophenyl)-1H-imidazol-1-yl]bicyclo[1.1.1]pentan-1-yl}-4-hydroxy-3,4-dihydro-2H-1-benzopyran-2-carboxamide

The methodologies described in Example 155C substituting Example 182B for Example 155B gave the title compound. ¹H NMR (500 MHz, DMSO-d₆) δ ppm 8.91 (s, 1H), 7.83 (d, J=1.3 Hz, 1H), 7.81-7.71 (m, 3H), 7.46-7.36 (m, 3H), 7.22 (dd, J=8.7, 2.6 Hz, 1H), 6.91 (d, J=8.7 Hz, 1H), 5.72 (d, J=6.3 Hz, 1H), 4.85-4.81 (m, 1H), 4.67 (dd, J=12.0, 2.3 Hz, 1H), 2.54 (s, 6H), 2.42-2.39 (m, 1H), 1.74 (q, J=11.3 Hz, 1H); MS (ESI+) m/z 470/472 (³⁵Cl/³⁷Cl, M+H)⁺.

### Example 183: (2R,4R)-6-chloro-N-{3-[4-(4-chloro-3-fluorophenyl)-1H-imidazol-1-yl]bicyclo[1.1.1]pentan-1-yl}-4-hydroxy-3,4-dihydro-2H-1-benzopyran-2-carboxamide (Compound 282)

### Example 183A: Tert-Butyl (3-(4-(4-chloro-3-fluorophenyl)-1H-imidazol-1-yl)bicyclo[1.1.1]pentan-1-yl)carbamate

The methodologies described in Example 49A substituting 4-chloro-3-fluorobenzaldehyde for 3,4-difluorobenzaldehyde and purifying by column chromatography on silica gel (0-100% ethyl acetate in isohexane) gave the title intermediate. ¹H NMR (500 MHz, DMSO-d₆) δ ppm 7.89 (s, 1H), 7.78 (d, J=1.3 Hz, 1H), 7.72 (dd, J=11.0, 1.9 Hz, 1H), 7.63 (dd, J=8.4, 1.9 Hz, 1H), 7.56 (t, J=8.1 Hz, 1H), 2.41 (s, 6H), 1.41 (s, 9H); MS (ESI+) m/z 378 (M+H)⁺.

### Example 183B: 3-(4-(4-chloro-3-fluorophenyl)-1H-imidazol-1-yl)bicyclo[1.1.1]pentan-1-amine, Trifluoroacetic Acid

The methodologies described in Example 21B substituting Example 183A for Example 21A gave the title intermediate as a trifluoroacetic acid salt. ¹H NMR (500 MHz, DMSO-d₆) δ ppm 8.85 (s, 2H), 8.09 (s, 1H), 8.04 (d, J=1.3 Hz, 1H), 7.75-7.72 (m, 1H), 7.64-7.59 (m, 2H), 2.54 (s, 6H); MS (ESI+) m/z 278 (M+H)⁺.

### Example 183C: (2R,4R)-6-chloro-N-{3-[4-(4-chloro-3-fluorophenyl)-1H-imidazol-1-yl]bicyclo[1.1.1]pentan-1-yl}-4-hydroxy-3,4-dihydro-2H-1-benzopyran-2-carboxamide

The methodologies described in Example 155C substituting Example 183B for Example 155B gave the title compound. ¹H NMR (500 MHz, DMSO-d₆) δ ppm 8.92 (s, 1H), 7.93 (d, J=1.3 Hz, 1H), 7.82 (d, J=1.3 Hz, 1H), 7.76-7.71 (m, 1H), 7.64 (d, J=8.9 Hz, 1H), 7.56 (t, J=8.1 Hz, 1H), 7.40 (d, J=2.7 Hz, 1H), 7.22 (dd, J=8.9, 2.7 Hz, 1H), 6.91 (d, J=8.7 Hz, 1H), 5.73 (d, J=6.3 Hz, 1H), 4.86-4.81 (m, 1H), 4.69-4.65 (m, 1H), 2.54 (s, 6H), 2.42-2.38 (m, 1H), 1.74 (q, J=11.7 Hz, 1H); MS (ESI+) m/z 488/490 (³⁵Cl/³⁷Cl, M+H)⁺.

### Example 184: (2S,4R)-6-chloro-4-hydroxy-N-(3-{5-[cis-3-(trifluoromethoxy)cyclobutyl]-1,2-oxazol-3-yl}bicyclo[1.1.1]pentan-1-yl)-3,4-dihydro-2H-1-benzopyran-2-carboxamide (Compound 283)

The title compound was prepared using the methods described for the synthesis of Example 131D, substituting the product from Example 135C for the product from Example 131C. ¹H NMR (500 MHz, DMSO-d₆) δ ppm 8.85 (s, 1H), 7.32 (d, J=2.5 Hz, 1H), 7.25 (dd, J=9.0, 2.5 Hz, 1H), 6.94 (d, J=9.0 Hz, 1H), 6.42 (s, 1H), 5.68 (br. s, 1H), 4.85 (p, J=7.5 Hz, 1H), 4.61-4.53 (m, 2H), 2.83-2.75 (m, 2H), 2.41-2.29 (m, 9H), 2.14-2.07 (m, 1H), 1.95-1.86 (m, 1H); MS (ESI) m/z 497 (M−H)⁻.

### Example 185: (2R,4R)-6-chloro-4-hydroxy-N-(3-{3-[trans-3-(trifluoromethoxy)cyclobutyl]-1,2-oxazol-5-yl}bicyclo[1.1.1]pentan-1-yl)-3,4-dihydro-2H-1-benzopyran-2-carboxamide (Compound 284)

### Example 185A: Cis-Benzyl 3-hydroxycyclobutanecarboxylate

To a stirred solution of benzyl 3-oxocyclobutanecarboxylate (5 g, 24.48 mmol), at −78° C. under an atmosphere of nitrogen, was added 1.0M lithium tri-tert-butoxyaluminum hydride in tetrahydrofuran (26.9 mL, 26.9 mmol) dropwise over 30 minutes and the resultant reaction mixture was stirred for 3 hour at this temperature. The reaction mixture was quenched with saturated NH₄Cl (aqueous) (50 mL) and extracted with ethyl acetate (2×50 mL). The combined organic fractions were dried over Na₂SO₄, filtered, and concentrated in vacuo. The residue was purified by chromatography on silica gel (24 g cartridge, dichloromethane loading, 0-100% ethyl acetate/isohexane) to afford the title compound (4.3 g, 20.43 mmol, 83% yield). ¹H NMR (500 MHz, DMSO-d₆) δ ppm 7.42-7.30 (m, 5H), 5.20 (d, J=6.9 Hz, 1H), 5.09 (s, 2H), 4.06-3.92 (m, 1H), 2.62 (tt, J=10.0, 7.7 Hz, 1H), 2.41 (dddd, J=10.3, 9.4, 5.2, 2.5 Hz, 2H), 2.03-1.92 (m, 2H).

### Example 185B: Trans-Benzyl 3-(formyloxy)cyclobutanecarboxylate

To a solution of the product of Example 185A (100 mg, 0.485 mmol) and formic acid (0.022 mL, 0.582 mmol) in tetrahydrofuran (2 mL), at room temperature under nitrogen, was added triphenylphosphine (153 mg, 0.582 mmol) followed by diisopropyl azodicarboxylate (0.104 mL, 0.533 mmol) and the subsequent reaction mixture was stirred for 2 hours. The reaction mixture was concentrated in vacuo. The residue was purified by chromatography on silica gel (4 g cartridge, dichloromethane loading, 0-100% ethyl acetate/isohexane) to afford the title compound (38 mg, 0.159 mmol, 32.8% yield). ¹H NMR (500 MHz, DMSO-d₆) δ ppm 8.14 (s, 1H), 7.42-7.30 (m, 5H), 5.18-5.10 (m, 3H), 3.24-3.17 (m, 1H), 2.60-2.53 (m, 2H), 2.42-2.34 (m, 2H).

### Example 185C: Trans-Benzyl 3-hydroxycyclobutanecarboxylate

A solution of the product of Example 185B (378 mg, 1.62 mmol) in dimethylamine (2 M in tetrahydrofuran) (2.5 mL, 5.0 mmol) was stirred at room temperature for 18 hours. The reaction mixture was concentrated in vacuo to afford the crude product which was purified by chromatography on silica gel (4 g cartridge, dichloromethane loading, 0-100% ethyl acetate/isohexane) to afford the title compound (312 mg, 1.483 mmol, 92% yield). ¹H NMR (500 MHz, DMSO-d₆) δ ppm δ 7.42-7.30 (m, 5H), 5.18 (d, J=6.3 Hz, 1H), 5.10 (s, 2H), 4.33-4.19 (m, 1H), 3.04-2.94 (m, 1H), 2.44-2.33 (m, 2H), 2.15-2.04 (m, 2H).

### Example 185D: trans-3-(trifluoromethoxy)cyclobutanecarboxylic Acid

The title compound was synthesized using the same procedure as described in Example 25N through Example 250 substituting the product of Example 25M with the product of Example 185C. ¹H NMR (500 MHz, DMSO-d₆) δ ppm 7.43-7.30 (m, 5H), 5.13 (s, 2H), 4.92 (p, J=7.0 Hz, 1H), 3.25-3.17 (m, 1H), 2.64-2.51 (m, 4H); ¹⁹F NMR (471 MHz, DMSO-d₆) δ ppm −57.80.

### Example 185E: 3-(3-(trans-3-(trifluoromethoxy)cyclobutyl)isoxazol-5-yl)bicyclo[1.1.1]pentan-1-amine Trifluoroacetic Acid

The title compound was synthesized using the same procedures as described in Example 167A through Example 167F substituting the product of Example 25N with the product of Example 185D. MS (ESI⁺) m/z 289.1 (M+H)⁺.

### Example 185F: (2R,4R)-6-chloro-4-hydroxy-N-(3-{3-[trans-3-(trifluoromethoxy)cyclobutyl]-1,2-oxazol-5-yl}bicyclo[1.1.1]pentan-1-yl)-3,4-dihydro-2H-1-benzopyran-2-carboxamide

The title compound was synthesized using the same procedure as described in Example 167G substituting the product of Example 167F with the product of Example 185E. ¹H NMR (500 MHz, DMSO-d₆) δ ppm 8.83 (s, 1H), 7.38 (d, J=2.6 Hz, 1H), 7.21 (dd, J=8.7, 2.7 Hz, 1H), 6.89 (d, J=8.7 Hz, 1H), 6.48 (s, 1H), 5.71 (d, J=3.6 Hz, 1H), 5.05 (p, J=6.9 Hz, 1H), 4.81 (dd, J=16.1, 1.6 Hz, 1H), 4.62 (dd, J=12.0, 2.2 Hz, 1H), 3.61-3.55 (m, 1H), 2.72-2.64 (m, 2H), 2.55 (ddt, J=10.7, 7.0, 3.7 Hz, 1H), 2.41 (s, 7H), 2.40-2.34 (m, 1H), 1.71 (q, J=11.9 Hz, 1H); ¹⁹F NMR (471 MHz, DMSO-d₆) δ ppm −57.54.

### Example 186: N-(3-{[(2R,4R)-6-chloro-4-hydroxy-3,4-dihydro-2H-1-benzopyran-2-carbonyl]amino}bicyclo[11.1]pentan-1-yl)-2-phenyl-1,3-oxazole-5-carboxamide (Compound 285)

### Example 186A: Tert-Butyl (3-(2-phenyloxazole-5-carboxamido)bicyclo[1.1.1]pentan-1-yl)carbamate

The reaction and purification conditions described in Example 2B substituting tert-butyl (3-aminobicyclo[1.1.1]pentan-1-yl)carbamate for the product of Example 2A, and 2-phenyloxazole-5-carboxylic acid (Ark Pharm) for the product of Example 1B gave the title compound. MS (APCI⁺) m/z 370 (M+H)⁺.

### Example 186B: N-(3-[(2R,4R)-6-chloro-4-hydroxy-3,4-dihydro-2H-1-benzopyran-2-carbonyl]amino)bicyclo[1.1.1]pentan-1-yl)-2-phenyl-1,3-oxazole-5-carboxamide (Compound 285)

The product of Example 186A (40 mg, 0.108 mmol) was combined with trifluoroacetic acid (1.0 mL) and stirred at ambient temperature for 30 minutes. The mixture was concentrated under reduced pressure. Triethylamine (0.075 mL, 0.54 mmol), N,N-dimethylformamide (1 mL), the product of Example 1B (24.5 mg, 0.108 mmol) and 1-[bis(dimethylamino)methylene]-1H-1,2,3-triazolo[4,5-b]pyridinium 3-oxid hexafluorophosphate (49.4 mg, 0.130 mmol) were added sequentially. The reaction mixture was stirred at ambient temperature for 1 hour and then partitioned between dichloromethane (2×30 mL) and saturated aqueous sodium bicarbonate (30 mL). The organic fractions were combined, dried over sodium sulfated, and concentrated under reduced pressure. The residue was taken up in methanol (5 mL) and sodium borohydride (49 mg, 1.3 mmol) was added in 3 portions over a period of 3 minutes. After stirring for another 20 minutes, saturated aqueous ammonium chloride (0.2 mL) was added and the resulting mixture was partitioned between dichloromethane (2×30 mL) and saturated aqueous sodium bicarbonate (30 mL). The organic layers were combined, dried over sodium sulfated, concentrated under reduced pressure, taken up in N,N-dimethylformamide (3 mL), and directly purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (26 mg, 0.054 mmol, 50% yield). ¹H NMR (500 MHz, DMSO-d₆) δ ppm 9.28 (s, 1H), 8.74 (s, 1H), 8.17-8.09 (m, 2H), 7.86 (s, 1H), 7.64-7.54 (m, 3H), 7.39 (dd, J=2.7, 0.9 Hz, 1H), 7.21 (ddd, J=8.7, 2.7, 0.7 Hz, 1H), 6.90 (d, J=8.7 Hz, 1H), 5.71 (d, J=4.4 Hz, 1H), 4.85-4.79 (m, 1H), 4.62 (dd, J=12.0, 2.3 Hz, 1H), 2.39 (s, 6H), 2.40-2.33 (m, 1H), 1.77-1.66 (m, 1H); MS (ESI⁺) m/z 480 (M+H)⁺.

### Example 187: (2R,4R)-6-chloro-N-[3-(2-{[cis-3-cyanocyclobutyl]oxy}-1,3-thiazol-4-yl)bicyclo[1.1.1]pentan-1-yl]-4-hydroxy-3,4-dihydro-2H-1-benzopyran-2-carboxamide (Compound 286)

### Example 187A: O-(cis-3-cyanocyclobutyl) carbamothioate

At 0° C., under atmosphere of nitrogen, sodium hydride (49.4 mg, 1.236 mmol) (60 weight % dispersion in mineral oil) was added to a solution of the product of Example 119A (100 mg, 1.030 mmol) in tetrahydrofuran (4 mL). The reaction mixture was stirred at 0° C. for 90 minutes before the addition of carbon disulfide (0.074 mL, 1.236 mmol). The reaction mixture was allowed to warm to room temperature and stirred for 23 hours. Iodomethane (0.077 mL, 1.236 mmol) was then added and the mixture was stirred at room temperature for 5 hours. Ammonium hydroxide (0.139 mL, 2.059 mmol) was added and the reaction mixture was stirred at room temperature overnight. Water (10 mL) was added and the suspension was extracted with dichloromethane (3×10 mL). The combined extracts were dried over MgSO₄, filtered and concentrated. The residue was purified by chromatography on silica gel (24 g cartridge, 0-100% ethyl acetate/isohexane) to give the title compound (50 mg, 0.314 mmol, 30.5% yield). ¹H NMR (500 MHz, DMSO-d₆) δ ppm 8.82 (s, 1H), 8.49 (s, 1H), 5.18 (p, J=7.4 Hz, 1H), 3.10-3.02 (m, 1H), 2.85-2.73 (m, 2H), 2.37-2.22 (m, 2H).

### Example 187B: cis-3-((4-(3-aminobicyclo[1.1.1]pentan-1-yl)thiazol-2-yl)oxy)cyclobutanecarbonitrile

At room temperature, the product of Example 187A (25.7 mg, 0.164 mmol) was added to a solution of the product of Example 181B (50 mg, 0.164 mmol) in ethanol (2 mL). The reaction mixture was stirred at 80° C. for 3 hour, and then was concentrated to give the crude tert-butyl (3-(2-(cis-3-cyanocyclobutoxy)thiazol-4-yl)bicyclo[1.1.1]pentan-1-yl)carbamate (59 mg, 0.226 mmol, 137% yield). The material was used in the next step without further purification. This crude tert-butyl (3-(2-(cis-3-cyanocyclobutoxy)thiazol-4-yl)bicyclo[1.1.1]pentan-1-yl)carbamate (120 mg, 0.332 mmol) was dissolved in dichloromethane (2 mL) and treated with trifluoroacetic acid (0.384 mL, 4.98 mmol). The reaction mixture was allowed to stir at room temperature overnight. Methanol (2.0 mL) was added followed by SCX resin (1.22 g), and the suspension was stirred for 1 hour. The solid was collected by filtration, washed with methanol (2×10 mL). The solid was washed with NH₃ (3.5 M in methanol, 10 mL), and this filtrate was concentrated to give the title compound (59 mg, 0.196 mmol, 59.2% yield) (83% over 2 steps). ¹H NMR (500 MHz, DMSO-d₆) δ ppm 6.61 (s, 1H), 5.05 (p, J=7.3 Hz, 1H), 3.14-3.06 (m, 1H), 2.87-2.80 (m, 2H), 2.46-2.39 (m, 2H), 1.89 (s, 6H).

### Example 187C: (2R,4R)-6-chloro-N-[3-(2-{[cis-3-cyanocyclobutyl]oxy}-1,3-thiazol-4-yl)bicyclo[1.1.1]pentan-1-yl]-4-hydroxy-3,4-dihydro-2H-1-benzopyran-2-carboxamide

At room temperature, the product of Example 3B (18 mg, 0.079 mmol) was added to a solution of the product of Example 187B (26.7 mg, 0.102 mmol) in N,N-dimethylformamide (1 mL). Additional N,N-dimethylformamide (0.5 mL) was used to transfer the remaining acid into the reaction mixture. N,N-Diisopropylethylamine (0.107 mL, 0.611 mmol) was then added, followed by 2,4,6-tripropyl-1,3,5,2,4,6-trioxatriphosphinane 2,4,6-trioxide (0.061 mL, 0.105 mmol) (50 weight % in N,N-dimethylformamide). The reaction mixture was stirred at room temperature over 3 nights. The reaction mixture was directly purified by preparative HPLC (Waters XBridgem Prep-C18, 5 μm column (19 mm×50 mm). A 40-70% gradient of acetonitrile (A) and 0.1% ammonium bicarbonate in water (B) was used over 7.5 minutes at a flow rate of 30 mL/minute) to give the title compound (15.5 mg, 0.031 mmol, 39.6% yield). ¹H NMR (500 MHz, DMSO-d₆) δ ppm 8.72 (s, 1H), 7.37 (d, J=2.7 Hz, 1H), 7.19 (dd, J=8.8, 2.7 Hz, 1H), 6.88 (d, J=8.7 Hz, 1H), 6.73 (s, 1H), 5.71 (d, J=6.3 Hz, 1H), 5.07 (p, J=7.2 Hz, 1H),4.80 (dt, J=10.6, 6.2 Hz, 1H),4.59 (dd, J=11.9, 2.2 Hz, 1H), 3.11 (p, J=8.9 Hz, 1H), 2.89-2.78 (m, 2H), 2.47-2.40 (m, 2H), 2.23 (s, 6H), 1.68 (q, J=12.0 Hz, 1H).

### Example 188: (2R,4R)-6-chloro-4-hydroxy-N-(3-{4-[cis-3-(trifluoromethoxy)cyclobutyl]-1H-imidazol-1-yl}bicyclo[1.1.1]pentan-1-yl)-3,4-dihydro-2H-1-benzopyran-2-carboxamide (Compound 287)

### Example 188A: cis-N-methoxy-N-methyl-3-(trifluoromethoxy)cyclobutanecarboxamide

The title compound was prepared using the methods described for the synthesis of Example 181A, substituting the product from Example 250 for 3-((tert-butoxycarbonyl)amino)bicyclo[1.1.1]pentane-1-carboxylic acid. ¹H NMR (500 MHz, DMSO-d₆) δ ppm 1H NMR (500 MHz, DMSO-d₆) δ 4.80 (p, J=7.5 Hz, 1H), 3.64 (s, 3H), 3.19-3.03 (m, 4H), 2.58-2.52 (m, 2H), 2.34-2.24 (m, 2H).

### Example 188B: cis-3-(trifluoromethoxy)cyclobutanecarbaldehyde

The title compound was prepared using the methods described for the synthesis of Example 128f, substituting the product from Example 188A for the product of Example 128e. The crude product was used without any analysis (assumed quantitative yield).

### Example 188C: Tert-Butyl (3-(4-(cis-3-(trifluoromethoxy)cyclobutyl)-1H-imidazol-1-yl)bicyclo[1.1.1]pentan-1-yl)carbamate

To a solution of the product of Example 188B (510 mg, 3.03 mmol) in ethanol/tetrahydrofuran (2:1, 20 mL) was added dropwise 1-((isocyanomethyl)sulfonyl)-4-methylbenzene (592 mg, 3.03 mmol) and sodium cyanide (28 mg, 0.57 mmol) dissolved in a small amount of water. The mixture was then stirred at ambient temperature for 3 hours. After this time, the reaction mixture was concentrated under reduced pressure and to the resulting residue was added dichloromethane (10 mL). The solution was then dried over MgSO₄, filtered and concentrated under reduced pressure. To the crude intermediate (1 g, 2.75 mmol) was added tert-butyl (3-aminobicyclo[1.1.1]pentan-1-yl)carbamate (500 mg, 2.52 mmol) and xylene (10 mL). The reaction mixture was then heated at 135° C. for 16 hours and was then concentrated under reduced pressure. The residue was purified by chromatography on silica gel (0-100% ethyl acetate/isohexane) to afford the title compound (66 mg, 6% yield). ¹H NMR (500 MHz, DMSO-d₆) δ ppm 7.81-7.64 (m, 1H), 7.56 (s, 1H), 7.03 (s, 1H), 4.77 (p, J=7.6 Hz, 1H), 3.00-2.90 (m, 1H), 2.64-2.57 (m, 2H), 2.42-2.23 (m, 8H), 1.40 (s, 9H); MS (ESI) m/z 388 (M+H)⁺.

### Example 188D: 3-(4-(cis-3-(trifluoromethoxy)cyclobutyl)-1H-imidazol-1-yl)bicyclo[1.1.1]pentan-1-amine

The title compound was prepared using the methods described for the synthesis of Example 119F, substituting the product of Example 119E with the product of Example 188C. ¹H NMR (500 MHz, DMSO-d₆) δ ppm 7.51 (s, 1H), 6.99 (s, 1H), 4.76 (p, J=7.6 Hz, 1H), 2.99-2.90 (m, 1H), 2.65-2.56 (m, 2H), 2.41 (s, 2H), 2.34-2.25 (m, 2H), 2.11 (s, 6H); MS (ESI) m/z 288 (M+H)⁺.

### Example 188E: (2R,4R)-6-chloro-4-hydroxy-N-(3-{4-[cis-3-(trifluoromethoxy)cyclobutyl]-1H-imidazol-1-yl}bicyclo[1.1.1]pentan-1-yl)-3,4-dihydro-2H-1-benzopyran-2-carboxamide

The title compound was prepared using the methods described for the synthesis of Example 141E, substituting the product of Example 141E with the product of Example 188D. ¹H NMR (500 MHz, DMSO-d₆) δ ppm 8.90 (s, 1H), 7.60 (s, 1H), 7.39 (d, J=2.7 Hz, 1H), 7.22 (dd, J=8.6, 2.7 Hz, 1H), 7.09 (s, 1H), 6.89 (d, J=8.7 Hz, 1H), 5.74 (d, J=6.3 Hz, 1H), 4.86-4.74 (m, 2H), 4.68-4.62 (m, 1H), 3.00-2.92 (m, 1H), 2.65-2.58 (m, 2H), 2.47 (s, 6H), 2.41-2.35 (m, 1H), 2.35-2.27 (m, 2H), 1.72 (q, J=11.9 Hz, 1H); MS (ESI) m/z 498 (M+H)⁺.

### Example 189: (2R,4R)-6-chloro-4-hydroxy-N-(3-{5-[cis-3-(trifluoromethoxy)cyclobutyl]-1,3-oxazol-2-yl}bicyclo[1.1.1]pentan-1-yl)-3,4-dihydro-2H-1-benzopyran-2-carboxamide (Compound 288)

### Example 189A: Tert-Butyl (3-((2-oxo-2-((cis)-3-(trifluoromethoxy)cyclobutyl)ethyl)carbamoyl)bicyclo[1.1.1]pentan-1-yl)carbamate

The methodologies described in Example 193E substituting 3-((tert-butoxycarbonyl)amino)bicyclo[1.1.1]pentane-1-carboxylic acid (PharmaBlock) for (2S,5R)-5-((tert-butoxycarbonyl)amino)tetrahydro-2H-pyran-2-carboxylic acid gave the title intermediate. ¹H NMR (500 MHz, DMSO-d₆) δ ppm 8.09 (t, J=5.9 Hz, 1H), 7.55 (s, 1H), 4.81 (p, J=7.5 Hz, 1H), 3.85 (d, J=5.8 Hz, 2H), 3.02-2.92 (m, 1H), 2.49-2.36 (m, 2H), 2.31-2.17 (m, 2H), 2.10 (s, 6H), 1.37 (s, 9H).

### Example 189B: 3-(4-(cis-3-(trifluoromethoxy)cyclobutyl)oxazol-2-yl)bicyclo[1.1.1]pentan-1-amine

The methodologies described in Example 193F substituting Example 189A for Example 193E gave the title intermediate. ¹H NMR (500 MHz, CDCl₃) δ ppm 6.68 (d, J=7.9 Hz, 1H), 4.61 (p, J=7.6 Hz, 1H), 3.06 (tt, J=10.1, 7.4 Hz, 1H), 2.79-2.69 (m, 2H), 2.45 (t, J=8.7 Hz, 2H), 2.24 (s, 6H); MS (ESI+) m/z 289 (M+H)⁺.

### Example 189C: (2R,4R)-6-chloro-4-hydroxy-N-(3-{5-[cis-3-(trifluoromethoxy)cyclobutyl]-1,3-oxazol-2-yl}bicyclo[1.1.1]pentan-1-yl)-3,4-dihydro-2H-1-benzopyran-2-carboxamide

The methodologies described in Example 155C substituting Example 189B for Example 155B gave the title compound. ¹H NMR (500 MHz, DMSO-d₆) δ ppm 8.83 (s, 1H), 7.39 (dd, J=2.7, 1.0 Hz, 1H), 7.21 (dd, J=8.7, 2.7 Hz, 1H), 6.94 (d, J=0.8 Hz, 1H), 6.89 (d, J=8.7 Hz, 1H), 5.72 (s, 1H), 4.82 (p, J=7.3 Hz, 2H), 4.62 (dd, J=12.0, 2.3 Hz, 1H), 3.22-3.16 (m, 1H), 2.81-2.68 (m, 2H), 2.41 (s, 6H), 2.37-2.30 (m, 3H), 1.71 (d, J=11.9 Hz, 1H); MS (ESI+) m/z 499 (M+H)⁺.

### Example 190: (2R,4R)-6-chloro-4-hydroxy-N-(3-{5-[cis-3-(trifluoromethoxy)cyclobutyl]-1H-imidazol-2-yl}bicyclo[1.1.1]pentan-1-yl)-3,4-dihydro-2H-1-benzopyran-2-carboxamide (Compound 289)

### Example 190A: Tert-Butyl (3-((2-oxo-2-((cis)-3-(trifluoromethoxy)cyclobutyl)ethyl)carbamoyl)bicyclo[1.1.1]pentan-1-yl)carbamate

The methodologies described in Example 193E substituting 3-((tert-butoxycarbonyl)amino)bicyclo[1.1.1]pentane-1-carboxylic acid (PharmaBlock) for (2S,5R)-5-((tert-butoxycarbonyl)amino)tetrahydro-2H-pyran-2-carboxylic acid and decreasing the reaction time from 3 days to 16 hours gave the title intermediate. ¹H NMR (500 MHz, DMSO-d₆) δ ppm 8.14-8.07 (m, 1H), 7.60-7.51 (m, 1H), 4.82 (p, J=7.4 Hz, 1H), 3.85 (d, J=5.3 Hz, 2H), 3.01-2.95 (m, 1H), 2.29-2.16 (m, 2H), 2.16-1.88 (s, 6H), 1.38 (s, 9H).

### Example 190B: Tert-Butyl (3-(5-((cis)-3-(trifluoromethoxy)cyclobutyl)-1H-imidazol-2-yl)bicyclo[1.1.1]pentan-1-yl)carbamate

To a stirred solution of Example 190A (0.250 g, 0.440 mmol) in xylene (2.5 mL) was added ammonium acetate (0.678 g, 8.80 mmol) at ambient temperature, and the reaction mixture was heated at 140° C. for 2 hours. Then the reaction mixture was cooled to ambient temperature and the solvent was removed under reduced pressure. The residue was purified by column chromatography on silica gel (0-10% 0.7 N NH₃ in methanol/dichloromethane) to afford the title intermediate (41 mg, 0.095 mmol, 22% yield). ¹H NMR (500 MHz, DMSO-d₆) δ ppm 11.60 (s, 1H), 7.21 (s, 1H), 6.87 (m, 1H), 4.79-4.67 (m, 1H), 2.98-2.82 (m, 1H), 2.60-2.55 (m, 2H), 2.34-2.19 (m, 2H), 2.16 (s, 6H), 1.39 (s, 9H).

### Example 190C: 3-(5-((cis)-3-(trifluoromethoxy)cyclobutyl)-1H-imidazol-2-yl)bicyclo[1.1.1]pentan-1-amine

To a solution of Example 190B (41 mg, 0.11 mmol) in dichloromethane (1.0 mL) was added trifluoroacetic acid (0.50 mL, 6.5 mmol) and the reaction mixture stirred at ambient temperature for 16 hours and then was diluted with methanol (15 mL). SCX resin (1 g) was added and the reaction mixture was stirred for 30 minutes. The mixture was loaded onto SCX resin (2 g), washed with methanol (3×10 mL) and eluted with 0.7 M NH₃ in methanol (3×10 mL) to afford the title intermediate (18 mg, 0.056 mmol, 53% yield). ¹H NMR (500 MHz, CDCl₃) δ ppm 6.70 (s, 1H), 4.64-4.54 (m, 1H), 3.18-2.96 (m, 1H), 2.81-2.70 (m, 2H), 2.35-2.28 (m, 2H), 2.07 (s, 6H); MS (ESI+) m/z 288 (M+H)⁺.

### Example 190D: (2R,4R)-6-chloro-1-hydroxy-N-(3-{S-[cis-3-(tri fluoromethoxy)cyclobutyl]-1H-imidazol-2-yl}bicyclo[1.1.1]pentan-1-yl)-3,4-dihydro-2H-1-benzopyran-2-carboxamide

The methodologies described in Example 155C substituting Example 190C for Example 155B gave the title compound. ¹H NMR (500 MHz, DMSO-d₆) δ ppm 11.72 (d, J=64.0 Hz, 1H), 8.73 (s, 1H), 7.39 (dd, J=2.8, 1.0 Hz, 1H), 7.21 (dd, J=8.7, 2.8 Hz, 1H), 6.98-6.76 (m, 2H), 5.71 (s, 1H), 4.91-4.69 (m, 2H), 4.61 (dd, J=12.0, 2.2 Hz, 1H), 3.03-2.84 (m, 1H), 2.62-2.57 (m, 2H), 2.40-2.35 (m, 2H), 2.34-2.30 (m, 7H), 1.71 (d, J=11.9 Hz, 1H); MS (ESI⁺) m/z 498 (M+H)⁺.

### Example 191: (2R,4R)-6-chloro-N-[3-(4-cyclobutyl-1H-pyrazol-1-yl)bicyclo[1.1.1]pentan-1-yl]-4-hydroxy-3,4-dihydro-2H-1-benzopyran-2-carboxamide (Compound 290)

### Example 191A: Methyl 3-(4-cyclobutyl-1H-pyrazol-1-yl)bicyclo[1.1.1]pentane-1-carboxylate

The reaction and purification conditions described in Example 136A substituting 4-cyclobutyl-1H-pyrazole (Combi-Blocks) for 5-chloro-1H-indazole gave the title compound. MS (APCI⁺) m/z 265 (M+H)⁺.

### Example 191B: 3-(4-cyclobutyl-1H-pyrazol-1-yl)bicyclo[1.1.1]pentane-1-carboxylic Acid

The product of Example 191A (96 mg, 0.39 mmol) was combined with methanol (2 mL) and aqueous sodium hydroxide (2.5 M, 1.0 mL) was added. After stirring at ambient temperature for 1 hour, the reaction mixture was partitioned between dichloromethane (2×50 mL) and aqueous citric acid (10 w/w %, 50 mL). The organic layers were combined and dried over sodium sulfate and concentrated under reduced pressure to give the title compound (91 mg, 0.39 mmol, 100% yield). MS (APCI⁺) m/z 233 (M+H)⁺.

### Example 191C: 2-(trimethylsilyl)ethyl (3-(4-cyclobutyl-1H-pyrazol-1-yl)bicyclo[1.1.1]pentan-1-yl)carbamate

The reaction and purification conditions described in Example 125C substituting the product of Example 191B for the product of Example 125B gave the title compound. ¹H NMR (400 MHz, DMSO-d₆) δ ppm 7.91 (s, 1H), 7.57 (d, J=0.7 Hz, 1H), 7.34 (d, J=0.8 Hz, 1H), 4.08-3.98 (m, 2H), 3.32-3.26 (m, 1H), 2.42 (d, J=62.4 Hz, 6H), 2.28-2.17 (m, 2H), 2.03-1.75 (m, 4H), 0.99-0.85 (m, 2H), 0.02 (s, 9H); MS (APCI⁺) m/z 348 (M+H)⁺.

### Example 191D: (2R,4R)-6-chloro-N-[3-(4-cyclobutyl-1H-pyrazol-1-yl)bicyclo[1.1.1]pentan-1-yl]-4-hydroxy-3,4-dihydro-2H-1-benzopyran-2-carboxamide

The reaction and purification conditions described in Example 1C substituting the product of Example 191C for the product of Example 1A, and the product of Example 3B for the product of Example 1B gave the title compound. ¹H NMR (400 MHz, DMSO-d₆) δ ppm 8.86 (s, 1H), 7.60 (s, 1H), 7.41-7.33 (m, 2H), 7.21 (dd, J=8.7, 2.7 Hz, 1H), 6.89 (d, J=8.7 Hz, 1H), 5.71 (s, 1H), 4.84-4.80 (m, 1H), 4.64 (dd, J=11.9, 2.3 Hz, 1H), 3.37-3.26 (m, 1H), 2.47 (s, 6H), 2.37 (ddd, J=12.7, 5.9, 2.5 Hz, 1H), 2.28-2.17 (m, 2H), 2.04-1.77 (m, 4H), 1.77-1.66 (m, 1H); MS (APCI⁺) m/z 414 (M+H)⁺.

### Example 192: (2S,4R)-6-chloro-N-[3-(4-cyclobutyl-1H-pyrazol-1-yl)bicyclo[1.1.1]pentan-1-yl]-4-hydroxy-3,4-dihydro-2H-1-benzopyran-2-carboxamide (Compound 291)

The reaction and purification conditions described in Example 1C substituting the product of Example 191C for the product of Example 1A, and the product of Example 73B for the product of Example 1B gave the title compound. ¹H NMR (400 MHz, DMSO-d₆) δ ppm 8.92 (s, 1H), 7.60 (t, J=0.7 Hz, 1H), 7.36-7.35 (m, 1H), 7.32 (d, J=2.7 Hz, 1H), 7.26 (dd, J=8.7, 2.7 Hz, 1H), 6.94 (d, J=8.8 Hz, 1H), 5.63 (s, 1H), 4.63-4.55 (m, 2H), 3.39-3.25 (m, 1H), 2.47 (s, 6H), 2.27-2.18 (m, 2H), 2.15-2.08 (m, 1H), 2.03-1.75 (m, 5H); MS (APCI⁺) m/z 414 (M+H)⁺.

### Example 193: (2R,4R)-6-chloro-4-hydroxy-N-[(3R,6S)-6-{5-[3-(trifluoromethoxy)cyclobutyl]-1,3-oxazol-2-yl}oxan-3-yl]-3,4-dihydro-2H-1-benzopyran-2-carboxamide (Compound 292)

### Example 193A: (cis)-N-methoxy-N-methyl-3-(trifluoromethoxy)cyclobutanecarboxamide

To a cooled (0° C.) solution of 1-[bis(dimethylamino)methylene]-1H-1,2,3-triazolo[4,5-b]pyridinium 3-oxid hexafluorophosphate (HATU, 1.42 g, 3.72 mmol), N-ethyl-N-isopropylpropan-2-amine (1.7 mL, 9.9 mmol) and N,O-dimethylhydroxylamine hydrochloride (0.291 g, 2.98 mmol) in dichloromethane (12 mL) and N,N-dimethylformamide (5 mL) was added Example 25N (0.457 g, 2.48 mmol) and the reaction mixture was stirred at 0° C. for 1 hour. Then N,N-dimethylformamide (2 mL) was added until the mixture was a homogeneous solution. The reaction mixture was then stirred at ambient temperature for 24 hours. After this time, the reaction mixture was diluted with ethyl acetate (150 mL) and washed with hydrogen chloride (1 M, 75 mL), saturated aqueous sodium bicarbonate (75 mL) and brine (100 mL×3). The organic phase was dried over MgSO₄, filtered, and concentrated in vacuo to give the title intermediate (0.643 g, 2.49 mmol, quantitative yield). ¹H NMR (500 MHz, DMSO-d₆) δ ppm 4.79 (p, J=7.5 Hz, 1H), 3.63 (s, 3H), 3.22-2.93 (m, 4H), 2.56-2.51 (m, 2H), 2.32-2.24 (m, 2H).

### Example 193B: 1-((cis)-3-(trifluoromethoxy)cyclobutyl)ethanone

To a cooled (0° C.) solution of Example 193A (1.00 g, 4.40 mmol) in tetrahydrofuran (10 mL) was added methylmagnesium bromide (3 M in diethyl ether, 4.4 mL, 13 mmol) dropwise. The reaction mixture was stirred at ambient temperature overnight. Then the reaction mixture was quenched with HCl (0.5 M, aqueous, 50 mL) and extracted with dichloromethane (3×50 mL). The organic layers were combined and concentrated in vacuo without fully evaporating the solvent due to compound volatility to give the title intermediate (0.85 g, 2.0 mmol, 45% yield). ¹H NMR (500 MHz, DMSO-d₆) δ ppm 4.77 (p, J=7.5 Hz, 1H), 2.97-2.89 (m, 1H), 2.56-2.50 (m, 2H), 2.25-2.17 (m, 2H), 2.07 (s, 3H).

### Example 193C: 2-bromo-1-((cis)-3-(trifluoromethoxy)cyclobutyl)ethanone

To a solution of Example 193B (0.500 g, 1.15 mmol) in methanol (9 mL) was added a solution of HBr (48% aqueous solution, 0.07 mL, 1.3 mmol) in methanol (2 mL), followed by a solution of Br₂ (0.07 mL, 1.3 mmol) in methanol (9 mL) dropwise. The reaction mixture was stirred at ambient temperature overnight. The reaction mixture was then poured into ice water (50 mL) and extracted with dichloromethane (3×50 mL). The combined organic fractions were then washed with brine (3×50 mL), concentrated in vacuo. The residue was purified by silica gel column chromatography (0-100% ethyl acetate in isohexane) to give the title intermediate (0.32 g, 0.91 mmol, 79% yield). ¹H NMR (500 MHz, DMSO-d₆) δ ppm 4.81 (p, J=7.5 Hz, 1H), 4.38 (s, 2H), 3.18-3.11 (m, 1H), 2.63-2.52 (m, 2H), 2.35-2.23 (m, 2H).

### Example 193D: 2-amino-1-((cis)-3-(trifluoromethoxy)cyclobutyl)ethanone Hydrochloride

To a solution of Example 193C (1.8 g, 6.9 mmol) in acetonitrile (43 mL) was added N-formylformamide (sodium salt, 0.76 g, 7.9 mmol) and the reaction mixture was stirred at ambient temperature for 1.5 days. Then the volatiles were removed and the residue was dissolved in ethanol (85 mL) and hydrogen chloride (4 N in dioxane, 17 mL, 68 mmol) was added. The reaction mixture was stirred at ambient temperature for 2 hours. Then the volatiles were removed. The crude residue was then triturated with tert-butyl methyl ether (3×15 mL) to give the title intermediate. ¹H NMR (500 MHz, DMSO-d₆) δ ppm 8.21 (s, 3H), 4.85 (p, J=7.5 Hz, 1H),3.94 (s, 2H), 3.13-3.04 (m, 1H), 2.65-2.57 (m, 2H), 2.37-2.23 (m, 2H); MS (ESI⁺) m/z 198 (M+H)⁺.

### Example 193E: Tert-Butyl ((3R,6S)-6-((2-oxo-2-((cis)-3-(trifluoromethoxy)cyclobutyl)ethyl)carbamoyl)tetrahydro-2H-pyran-3-yl)carbamate

To a mixture of (2S,5R)-5-((tert-butoxycarbonyl)amino)tetrahydro-2H-pyran-2-carboxylic acid (Astatech, 0.50 g, 2.1 mmol) and Example 193D (0.40 g, 1.7 mmol) in N,N-dimethylformamide (9.7 mL) was added Hunig's Base (N,N-diisopropylethylamine) (0.89 mL, 5.1 mmol) followed by HATU (1-[bis(dimethylamino)methylene]-1H-1,2,3-triazolo[4,5-b]pyridinium 3-oxid hexafluorophosphate) (0.98 g, 2.6 mmol). This reaction mixture was allowed to stir at ambient temperature for three days, and then the mixture was diluted with ethyl acetate (10 mL), washed with HCl (1 M, 5 mL), then sodium bicarbonate (saturated aqueous, 5 mL), and then brine (5 mL). The organic layer was then dried over Na₂SO₄, filtered, and concentrated in vacuo to afford the title intermediate. MS (APCI⁺) m/z 369 (M−tBu+H)⁺.

### Example 193F: (3R,6S)-6-(5-(cis-3-(trifluoromethoxy)cyclobutyl)oxazol-2-yl)tetrahydro-2H-pyran-3-amine

To Example 193E (0.72 g, 1.7 mmol) was added POCl₃ (6.9 mL, 74 mmol). This reaction mixture was allowed to stir at 40° C. for 2 hours. After this time, the reaction mixture was cooled to ambient temperature and concentrated. The resulting solid was then dissolved in methanol (10 mL) and filtered through an SCX-2 (strong cation exchange) resin, washing with methanol (3×10 mL). The product was then eluted with 1 N NH₃ in methanol (3-10 mL) and the volatiles were removed. The residue was diluted with N,N-dimethylformamide (2 mL) and water (0.5 mL), filtered, and purified by preparative HPLC (Waters XBridgem C18 5 μm OBD column, 30×100 mm, flow rate 40 mL/minute, 5-100% gradient of acetonitrile in 0.1% trifluoroacetic acid/water) to give the title intermediate (0.50 g, 1.7 mmol, 97% yield). MS (ESI⁺) 307 (M+H)⁺.

### Example 193G: (2R)-6-chloro-4-oxo-N-[(3R,6S)-6-{5-[cis-3-(trifluoromethoxy)cyclobutyl]-1,3-oxazol-2-yl}oxan-3-yl]-3,4-dihydro-2H-1-benzopyran-2-carboxamide

The methodologies described in Example 30D substituting the product of Example 1B for 3-(2-(4-chloro-3-fluorophenoxy)acetamido)bicyclo[1.1.1]pentane-1-carboxylic acid and substituting Example 193F for Example 30C gave the title intermediate. MS (APCI⁺) m/z 515 (M+H)⁺.

### Example 193H: (2R,4R)-6-chloro-4-hydroxy-N-[(3R,6S)-6-{5-[3-(trifluoromethoxy)cyclobutyl]-1,3-oxazol-2-yl}oxan-3-yl]-3,4-dihydro-2H-1-benzopyran-2-carboxamide

The methodologies described in Example 5 substituting Example 193G for Example 4 and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (600 MHz, DMSO-d₆) δ ppm 7.98 (d, J=8.0 Hz, 1H), 7.39 (dd, J=2.7, 1.0 Hz, 1H), 7.20 (ddd, J=8.8, 2.7, 0.7 Hz, 1H), 6.99 (d, J=0.7 Hz, 1H), 6.89 (d, J=8.7 Hz, 1H), 5.69 (d, J=6.3 Hz, 1H), 4.88-4.81 (m, 1H), 4.84-4.78 (m, 1H), 4.65 (dd, J=11.8, 2.3 Hz, 1H), 4.46 (dd, J=11.1, 2.4 Hz, 1H), 3.91-3.84 (m, 1H), 3.86-3.81 (m, 1H), 2.79-2.71 (m, 2H), 2.39-2.28 (m, 3H), 2.03-1.95 (m, 2H), 1.95-1.87 (m, 1H), 1.79-1.66 (m, 2H); MS (APCI⁺) m/z 517 (M+H)⁺.

### Example 194: (2R,4S)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide (Compound 293)

The product of Example 65 was processed as described in Example 73B substituting for the product of Example 73A. The crude product was further purified by preparative chiral HPLC [CHIRALPAK® IC 5 μm column, 20×250 mm, flow rate 20 mL/minute, 7% 2-propanol and 30% ethanol in heptane (isocratic gradient)] to give the title compound as the earlier eluting fraction. ¹H NMR (500 MHz, DMSO-d₆) δ ppm 8.71 (s, 1H), 8.36 (s, 1H), 7.31 (d, J=2.6 Hz, 1H), 7.24 (dd, J=8.8, 2.7 Hz, 1H), 6.92 (d, J=8.8 Hz, 1H), 5.62 (s, 1H), 4.57 (t, J=3.8 Hz, 1H), 4.54 (dd, J=10.9, 2.7 Hz, 1H), 4.48 (p, J=7.2 Hz, 1H), 3.72 (s, 2H), 3.72-3.66 (m, 1H), 2.78-2.69 (m, 2H), 2.25 (s, 6H), 2.18-2.11 (m, 2H), 2.08 (ddd, J=13.9, 3.8, 2.7 Hz, 1H), 1.89 (ddd, J=13.9, 10.9, 3.7 Hz, 1H); MS (APCI⁺) m/z 505 (M+H)⁺.

### Example 195: (2R,4R)-6-chloro-4-hydroxy-N-(3-{1-[cis-3-(trifluoromethoxy)cyclobutyl]-1H-imidazol-4-yl}bicyclo[1.1.1]pentan-1-yl)-3,4-dihydro-2H-1-benzopyran-2-carboxamide (Compound 294)

### Example 195A: Tert-Butyl (3-(4-tosyl-4,5-dihydrooxazol-5-yl)bicyclo[1.1.1]pentan-1-yl)carbamate

To a solution of the product of Example 128B (130 mg, 0.615 mmol) and 1-((isocyanomethyl)sulfonyl)-4-methylbenzene (120 mg, 0.615 mmol) in acetonitrile (1.25 mL) was added 1,8-diazabicycloundec-7-ene (9.28 μL, 0.062 mmol) and the reaction mixture stirred at room temperature for 45 minutes. The reaction mixture was then concentrated in vacuo to afford the crude title compound (298 mg, 0.615 mmol, 100% yield) that was used directly in the next step (assumed quantitative). MS (ESI⁺) m/z 407.2 (M+H)⁺.

### Example 195B: Tert-Butyl (3-(1-(cis-3-(trifluoromethoxy)cyclobutyl)-1H-imidazol-4-yl)bicyclo[1.1.1]pentan-1-yl)carbamate

The product of Example 106A (177 mg, 0.923 mmol) was partitioned between xylene (2.5 mL) and saturated aqueous potassium carbonate (2.0 mL). The phases were separated, and the aqueous was further extracted with xylene (2.5 mL). The combined xylene fractions were dried over Na₂SO₄, decanted, and then used as the reaction media. To the xylene solution was added the product of Example 195A (250 mg, 0.615 mmol) and the reaction mixture was heated via microwave irradiation using a silicon carbide heating element at 140° C. for 30 minutes. The reaction mixture was concentrated in vacuo. The residue was purified by chromatography on silica gel (24 g cartridge, dichloromethane loading, 0-10% methanol in dichloromethane) to afford the title compound (72 mg, 0.167 mmol, 27.2% yield). ¹H NMR (500 MHz, DMSO-d₆) δ ppm 7.62 (d, J=1.4 Hz, 1H), 7.50 (s, 1H), 7.14 (s, 1H), 4.69 (p, J=7.3 Hz, 1H), 4.39-4.28 (m, 1H), 2.90 (td, J=9.9, 7.0 Hz, 2H), 2.60-2.52 (m, 2H), 2.05 (s, 6H), 1.38 (s, 9H).

### Example 195C: 3-(1-(cis-3-(trifluoromethoxy)cyclobutyl)-1H-imidazol-4-yl)bicyclo[1.1.1]pentan-1-amine trifluoroacetic Acid

To the product of Example 195B (72 mg, 0.186 mmol) in dichloromethane (2.0 mL) at room temperature was added trifluoroacetic acid (0.215 mL, 2.79 mmol) and the reaction mixture was stirred for 2 hours. The volatiles were removed under vacuum and co-evaporated with toluene (3×10 mL) followed by co-evaporation with dichloromethane (minimum amount) and hexane (5 mL) to afford the title compound (100 mg, 0.204 mmol, 110% yield). MS (ESI⁺) m/z 288.1 (M+H)⁺.

### Example 195D: (2R,4R)-6-chloro-4-hydroxy-N-(3-{1-[cis-3-(trifluoromethoxy)cyclobutyl]-1H-imidazol-4-yl}bicyclo[1.1.1]pentan-1-yl)-3,4-dihydro-2H-1-benzopyran-2-carboxamide

The title compound was synthesized using the same procedure as described in Example 167G substituting the product of Example 167F with the product of Example 195C. ¹H NMR (500 MHz, DMSO-d₆) δ ppm 8.65 (s, 1H), 7.63 (d, J=1.4 Hz, 1H), 7.38 (dd, J=2.7, 1.0 Hz, 1H), 7.22-7.18 (m, 2H), 6.89 (d, J=8.7 Hz, 1H), 5.70 (d, J=5.1 Hz, 1H), 4.84-4.77 (m, 1H), 4.73-4.66 (m, 1H), 4.59 (dd, J=12.0, 2.2 Hz, 1H), 4.39-4.31 (m, 1H), 2.94-2.87 (m, 2H), 2.60-2.52 (m, 2H), 2.39-2.33 (m, 1H), 2.20 (s, 6H), 1.75-1.66 (m, 1H).

### Example 196: (2S,4S)-6-chloro-4-hydroxy-N-[3-(2-{[cis-3-(trifluoromethoxy)cyclobutyl]oxy}acetamido)bicyclo[1.1.1]pentan-1-yl]-3,4-dihydro-2H-1-benzopyran-2-carboxamide (Compound 295)

The reaction and purification conditions described in Example 186B substituting the product of Example 109A for the product of Example 186A, and the product of Example 10A for the product of Example 1B gave the title compound. ¹H NMR (600 MHz, DMSO-d₆) δ ppm 8.65 (s, 1H), 8.35 (s, 1H), 7.38 (dd, J=2.8, 1.0 Hz, 1H), 7.20 (ddd, J=8.7, 2.7, 0.7 Hz, 1H), 6.88 (d, J=8.7 Hz, 1H), 5.68 (s, 1H), 4.83-4.77 (m, 1H), 4.59 (dd, J=12.0, 2.3 Hz, 1H), 4.48 (p, J=7.1 Hz, 1H), 3.73 (s, 2H), 3.72-3.66 (m, 1H), 2.78-2.70 (m, 2H), 2.34 (ddd, J=12.9, 5.9, 2.3 Hz, 1H), 2.26 (s, 6H), 2.18-2.12 (m, 2H), 1.69 (ddd, J=12.9, 12.0, 10.8 Hz, 1H); MS (APCI⁺) m/z 487 (M−H₂O+H)⁺.

### Example 197: (2R)-6-chloro-4-oxo-N-[3-({(1RS,2SR)-2-[(trifluoromethoxy)methyl]cyclopropane-1-carbonyl}amino)bicyclo[1.1.1]pentan-1-yl]-3,4-dihydro-2H-1-benzopyran-2-carboxamide (Compound 296)

### Example 197A: 2-((benzyloxy)carbonyl)cyclopropanecarboxylic Acid

To a solution of 3-oxabicyclo[3.1.0]hexane-2,4-dione (25 g, 223 mmol) in tetrahydrofuran (250 mL) was added benzyl alcohol (72.4 g, 669 mmol) and triethylamine (67.7 g, 669 mmol). The reaction mixture was stirred at ambient temperature for 12 hours. Seven additional reactions were set up and run as described above. The reaction batches were combined and concentrated in vacuo. To the residue was added water (2 L), sodium carbonate solution to adjust the pH of the mixture to 8, and ethyl acetate (8×1 L). After separation, to the aqueous phase was added HCl (1 mol/L in water) to adjust the pH to 3. The aqueous phase was then extracted with ethyl acetate (3×2 L) and the combined organic phases were concentrated under reduced pressure to give the title intermediate (300 g, 1.23 mol, 69% yield). ¹H NMR (400 MHz, CDCl₃) δ ppm 1.37 (td, J=8.50, 5.08 Hz, 1H) 1.74 (td, J=6.84, 5.14 Hz, 1H) 2.05-2.29 (m, 2H) 5.15 (d, J=0.75 Hz, 2H) 7.30-7.44 (m, 5H).

### Example 197B: Benzyl 2-(hydroxymethyl)cyclopropanecarboxylate

To a solution of Example 197A (30 g, 123 mmol) in tetrahydrofuran (300 mL) at 0° C. under nitrogen was added borane dimethyl sulfide (24.5 mL, 245 mmol), and the reaction mixture was stirred at ambient temperature for 12 hours. Then the reaction mixture was cooled to 0° C., and the reaction mixture was quenched with methanol dropwise until gas evolution had ceased. Nine additional reactions were set up and run as described above and then combined. After bulk solvent removal, the resulting residue was concentrated under reduced pressure to give the title intermediate (230 g, 892 mmol, 73% yield). ¹H NMR (400 MHz, CDCl₃) δ ppm 1.07-1.23 (m, 2H) 1.53-1.72 (m, 1H) 1.84 (td, J=8.25, 5.75 Hz, 1H) 2.64 (br s, 1H) 3.75 (dd, J=11.82, 8.19 Hz, 1H) 3.94 (dd, J=11.88, 5.25 Hz, 1H) 5.15 (s, 2H) 7.29-7.45 (m, 5H).

### Example 197C: Benzyl 2-((trifluoromethoxy)methyl)cyclopropanecarboxylate

A mixture of silver trifluoromethanesulfonate (20 g, 78 mmol), (1-chloromethyl-4-fluoro-1,4-diazoniabicyclo[2.2.2]octane bis(tetrafluoroborate)) (Selectfluor™, 10 g, 29 mmol), and potassium fluoride (4.5 g, 78 mmol) in a flask wrapped with aluminum foil was cooled in a water bath. To this reaction mixture was added a solution of Example 197B (5 g, 19 mmol) in ethyl acetate (100 mL), followed by 2-fluoropyridine (5.0 mL, 58 mmol) and (trifluoromethyl)trimethylsilane (8.6 mL, 58 mmol) dropwise to keep the internal temperature lower than 10° C. The mixture was stirred at ambient temperature for 48 hours. Then the suspension was filtered through a pad of diatomaceous earth and the pad was washed with ethyl acetate (3×30 mL). The combined filtrates were concentrated and the residue was purified by column chromatography on silica gel (petroleum ether/ethyl acetate, 20:1) to give the title intermediate (2 g, 5.8 mmol, 30% yield). ¹H NMR (400 MHz, CDCl₃) δ ppm 1.14-1.25 (m, 2H) 1.64-1.76 (m, 1H) 1.92-2.00 (m, 1H) 4.10-4.16 (m, 1H) 4.33 (dd, J=10.96, 6.14 Hz, 1H) 5.08-5.30 (m, 2H) 7.30-7.42 (m, 5H).

### Example 197D: rac-(1R,2S)-2-(trifluoromethoxy)methyl/cyclopropane-1-carboxylic Acid

To a solution of Example 197C (10 g, 29 mmol) in tetrahydrofuran (90 mL) was added palladium hydroxide on carbon (4.1 g, 2.9 mmol, 20% weight, 50% water) at ambient temperature, and the reaction mixture was stirred under hydrogen (15 psi) for 12 hours. Then the reaction mixture was diatomaceous earth and the filter cake was washed with ethyl acetate (20 mL×3). The combined filtrates were concentrated to give a crude residue, which was purified by column chromatography on silica gel (petroleum ether:ethyl acetate=40:1) to give the title intermediate. ¹H NMR (400 MHz, DMSO-d₆) δ ppm 0.89-0.95 (m, 1H) 1.16 (td, J=8.25, 4.50 Hz, 1H) 1.64-1.75 (m, 1H) 1.76-1.84 (m, 1H) 4.14 (t, J=9.82 Hz, 1H) 4.40 (dd, J=10.44, 5.94 Hz, 1H) 12.40 (br s, 1H).

### Example 197E: Tert-Butyl [3-({rac-(1R,2S)-2-[(trifluoromethoxy)methyl]cyclopropane-1-carbonyl}amino)bicyclo[1.1.1]pentan-1-yl]carbamate

The methodologies described in Example 30D substituting Example 197D for 3-(2-(4-chloro-3-fluorophenoxy)acetamido)bicyclo[1.1.1]pentane-1-carboxylic acid, substituting tert-butyl (3-aminobicyclo[1.1.1]pentan-1-yl)carbamate (PharmaBlock) for Example 30C, and removing the HPLC purification gave the title intermediate, which was carried forward without purification. MS (APCI⁺) m/z 365 (M+H)⁺.

### Example 197F: rac-(1R,2S)—N-(3-aminobicyclo[1.1.1]pentan-1-yl)-2-[(trifluoromethoxy)methyl]cyclopropane-1-carboxamide

The methodologies described in Example 21B substituting Example 197E for Example 21A gave the title intermediate. MS (APCI⁺) m/z 265 (M+H)⁺.

### Example 197G: (2R)-6-chloro-4-oxo-N-[3-({(1RS,2SR)-2-[(trifluoromethoxy)methyl]cyclopropane-1-carbonyl}amino)bicyclo[1.1.1]pentan-1-yl]-3,4-dihydro-2H-1-benzopyran-2-carboxamide

The methodologies described in Example 30D substituting the product of Example 1B for 3-(2-(4-chloro-3-fluorophenoxy)acetamido)bicyclo[1.1.1]pentane-1-carboxylic acid and substituting Example 197F for Example 30C with extended reaction time to 3 days gave the title compound. ¹H NMR (600 MHz, DMSO-d₆) δ ppm 8.91 (s, 1H), 8.79 (s, 1H), 7.67-7.61 (m, 2H), 7.16 (dd, J=8.6, 0.6 Hz, 1H), 5.08 (t, J=7.1 Hz, 1H), 4.35 (dd, J=10.2, 6.3 Hz, 1H), 4.15 (dd, J=10.2, 8.8 Hz, 1H), 2.94 (d, J=7.1 Hz, 2H), 2.20 (q, J=0.9 Hz, 6H), 1.73 (td, J=8.1, 5.7 Hz, 1H), 1.57-1.50 (m, 1H), 1.03-0.96 (m, 1H), 0.87 (ddd, J=6.5, 5.7, 4.2 Hz, 1H); MS (APCI⁺) m/z 473 (M+H)⁺.

### Example 198: (2R,4R)-6-chloro-4-hydroxy-N-(4-{5-[cis-3-(trifluoromethoxy)cyclobutyl]-1,3-oxazol-2-yl}bicyclo[2.2.2]octan-1-yl)-3,4-dihydro-2H-1-benzopyran-2-carboxamide (Compound 297)

### Example 198A: Tert-Butyl (4-((2-oxo-2-((cis)-3-(trifluoromethoxy)cyclobutyl)ethyl)carbamoyl)bicyclo[2.2.2]octan-1-yl)carbamate

The methodologies described in Example 193E substituting 4-((tert-butoxycarbonyl)amino)bicyclo[2.2.2]octane-1-carboxylic acid (AChemBlock) for (2S,5R)-5-((tert-butoxycarbonyl)amino)tetrahydro-2H-pyran-2-carboxylic acid, decreasing the reaction time from 3 days to 2 hours, and including purification by preparative HPLC (Phenomenex® Luna® C8(2) 5 μm AXIA™ column (150 mm×30 mm) using a 30-100% gradient of acetonitrile (A) and 0.1% trifluoroacetic acid in water (B) over 25 minutes, at a flow rate of 50 mL/minute) gave the title intermediate. MS (APCI⁺) m/z 449 (M+H)⁺.

### Example 198B: 4-(5-(3-(trifluoromethoxy)cyclobutyl)oxazol-2-yl)bicyclo[2.2.2]octan-1-amine

The methodologies described in Example 193F substituting Example 198A for Example 193E and removing the resin work-up and HPLC purification gave the title intermediate, which was carried forward without purification. MS (ESI⁺) m/z 331 (M+H)⁺.

### Example 198C: (2R,4R)-6-chloro-4-hydroxy-N-(4-{5-[cis-3-(trifluoromethoxy)cyclobutyl]-1,3-oxazol-2-yl}bicyclo[2.2.2]octan-1-yl)-3,4-dihydro-2H-1-benzopyran-2-carboxamide

To a mixture of Example 198B (0.010 g, 0.030 mmol) and the product of Example 3B (0.010 g, 0.045 mmol) in N,N-dimethylformamide (0.31 mL) was added N-ethyl-N-isopropylpropan-2-amine (0.04 mL, 0.2 mmol) followed by 2,4,6-tripropyl-1,3,5,2,4,6-trioxatriphosphinane 2,4,6-trioxide (T3P®, 50% in N,N-dimethylformamide, 0.02 mL, 0.04 mmol). This reaction mixture was allowed to stir at ambient temperature for 7 hours, was diluted with N,N-dimethylformamide (2 mL) and water (0.5 mL), filtered, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.0014 g, 0.0026 mmol, 9% yield). ¹H NMR (500 MHz, DMSO-d₆) δ ppm 8.14 (s, 1H), 7.40-7.35 (m, 2H), 7.21-7.15 (m, 1H), 6.87 (d, J=8.7 Hz, 1H), 6.83 (d, J=0.7 Hz, 1H), 4.85-4.78 (m, 1H), 4.79 (s, 1H), 4.57 (dd, J=11.8, 2.3 Hz, 1H), 2.73-2.69 (m, 2H), 2.33-2.27 (m, 1H), 1.93 (m, 12H), 1.93-1.89 (m, 2H), 1.80-1.69 (m, 1H), 1.25 (d, J=10.9 Hz, 1H), 1.15 (s, 1H); MS (ESI⁺) m/z 541 (M+H)⁺.

### Example 199: (2R,4R)-6-chloro-4-hydroxy-N-(3-{1-[cis-3-(trifluoromethoxy)cyclobutyl]-1H-pyrazol-3-yl}bicyclo[1.1.1]pentan-1-yl)-3,4-dihydro-2H-1-benzopyran-2-carboxamide (Compound 298)

### Example 199A: (E)-tert-butyl (3-(3-(dimethylamino)acryloyl)bicyclo[1.1.1]pentan-1-yl)carbamate

To a solution tert-butyl (3-acetylbicyclo[1.1.1]pentan-1-yl)carbamate (500 mg, 2.219 mmol), the intermediate product of Example 181B, in N,N-dimethylformamide (8 mL) was added dimethylformamide dimethyl acetal (0.737 mL, 5.55 mmol) in a sealed vial. The reaction mixture was then stirred at 100° C. overnight. The reaction mixture was then cooled to ambient temperature and the volatiles were removed under reduced pressure to afford the title compound (589 mg, 76% yield). ¹H NMR (500 MHz, DMSO-d₆) δ ppm 7.51 (s, 1H), 7.45 (d, J=12.6 Hz, 1H), 5.06 (d, J=12.6 Hz, 1H), 3.06 (s, 3H), 2.77 (s, 3H), 2.01 (s, 6H), 1.38 (s, 9H).

### Example 199B: (cis-3-(benzyloxy)cyclobutyl)hydrazine

A mixture of 3-(benzyloxy)cyclobutanone (3 g, 17.02 mmol) and tert-butyl hydrazinecarboxylate (2.250 g, 17.02 mmol) in isohexane (50 mL) was heated under reflux overnight. The reaction mixture was concentrated in vacuo to afford tert-butyl 2-(3-(benzyloxy)cyclobutylidene)hydrazinecarboxylate (4.5 g, 91% yield). tert-Butyl 2-(3-(benzyloxy)cyclobutylidene)hydrazinecarboxylate (1 g, 3.44 mmol) was dissolved in tetrahydrofuran (10 mL) at ambient temperature. Borane dimethyl sulfide complex (1.044 mL, 10.33 mmol) was added and the reaction mixture was stirred at ambient temperature overnight. The reaction was quenched with 6 M aqueous HCl (10 mL). The solid was collected by filtration and discarded. The filtrate was concentrated in vacuo. 1 M aqueous HCl (10 mL) was added to the residue and the solid was filtered off. The filtrate was once more concentrated in vacuo to afford the title compound as the hydrochloric acid salt (0.788 g, 3.44 mmol, 100% yield).

### Example 199C: Tert-Butyl (3-(1-(cis-3-(benzyloxy)cyclobutyl)-1H-pyrazol-3-yl)bicyclo[1.1.1]pentan-1-yl)carbamate

To a solution of the product of Example 199B (732 mg, 3.20 mmol) in ethanol (10 mL) was added the product of Example 199A (459 mg, 1.637 mmol) and the reaction mixture stirred at 85° C. overnight and then left standing for 24 hours at ambient temperature. The reaction mixture was concentrated in vacuo. The crude product was purified by chromatography on silica gel (0-100% ethyl acetate/isohexane) to afford the title compound (59 mg, 8% yield). ¹H NMR (500 MHz, CDCl₃) δ ppm 7.45 (d, J=2.3 Hz, 1H), 7.39-7.31 (m, 5H), 6.12 (d, J=2.3 Hz, 1H), 4.99 (s, 1H), 4.49 (s, 2H), 4.48-4.37 (m, 1H), 3.97-3.86 (m, 1H), 2.95-2.86 (m, 2H), 2.44 (dddd, J=7.7, 6.5, 4.9, 2.6 Hz, 2H), 2.33 (s, 6H), 1.48 (s, 9H); MS (ESI) m/z 410 (M+H)⁺.

### Example 199D: Tert-Butyl (3-(1-((cis-3-hydroxycyclobutyl)-1H-pyrazol-3-yl)bicyclo[1.1.1]pentan-1-yl)-carbamate

To a solution of the product of Example 199C (59 mg, 0.144 mmol) in ethanol (2 mL) was added 10% Pd—C (25 mg, 0.012 mmol) and the reaction mixture was stirred at ambient temperature for 3 days under 5 bar of hydrogen atmosphere. The reaction mixture was filtered through a microfiber filter and the filtrate was concentrated in vacuo. The residue was purified by chromatography on silica gel (0-100% ethyl acetate/isohexane) to afford the title compound (29 mg, 50% yield). ¹H NMR (500 MHz, DMSO-d₆) δ ppm 7.64 (d, J=2.2 Hz, 1H), 7.54 (s, 1H), 6.05 (d, J=2.2 Hz, 1H), 5.24 (d, J=6.7 Hz, 1H), 4.30-4.20 (m, 1H), 3.96-3.85 (m, 1H), 2.72-2.62 (m, 2H), 2.29-2.20 (m, 2H), 2.11 (s, 6H), 1.39 (s, 9H); MS (ESI) ml 320 (M+H)⁺.

### Example 199E: Tert-Butyl (3-(1-((cis-3-(trifluoromethoxy)cyclobutyl)-1H-pyrazol-3-yl)bicyclo[1.1.1]-pentan-1-yl)carbamate

A mixture of silver(I) trifluoromethanesulfonate (63.0 mg, 0.245 mmol), potassium fluoride (21.10 mg, 0.363 mmol), and Selectfluor™ (48.2 mg, 0.136 mmol) was stirred under a nitrogen atmosphere, in a flask wrapped with aluminum foil, and cooled with a water bath. To this was slowly added a solution of the product of Example 199D (29 mg, 0.091 mmol) in ethyl acetate (1 mL) followed by slow addition of 2-fluoropyridine (0.023 mL, 0.272 mmol) and then trimethyl(trifluoromethyl)silane (0.040 mL, 0.272 mmol). The reaction mixture then stirred at ambient temperature overnight. The reaction mixture was filtered through a pad of diatomaceous earth washed with ethyl acetate (5 mL) and concentrated in vacuo. The residue was purified by chromatography on silica gel (0-50% ethyl acetate/isohexane) to afford the title compound (10 mg, 28% yield). ¹H NMR (500 MHz, CDCl₃) δ ppm 7.41 (d, J=2.2 Hz, 1H), 6.13 (d, J=2.3 Hz, 1H), 5.02 (s, 1H), 4.54 (p, J=7.3 Hz, 1H), 4.50-4.39 (m, 1H), 3.06-2.97 (m, 2H), 2.83-2.73 (m, 2H), 2.33 (s, 6H), 1.48 (s, 9H); MS (ESI) m/z 388 (M+H)⁺.

### Example 199F: (2R,4R)-6-chloro-4-hydroxy-N-(3-{1-[cis-3-(trifluoromethoxy)cyclobutyl]-1H-pyrazol-3-yl}bicyclo[1.1.1]pentan-1-yl)-3,4-dihydro-2H-1-benzopyran-2-carboxamide

The title compound was prepared using the methods described for the synthesis of Example 131D, substituting the product from Example 131C with the product of Example 199E and substituting the product of Example 73B with the product of Example 3B. ¹H NMR (500 MHz, DMSO-d₆) δ ppm 8.71 (s, 1H), 7.76 (d, J=2.3 Hz, 1H), 7.41-7.37 (m, 1H), 7.21 (dd, J=8.7, 2.7 Hz, 1H), 6.90 (d, J=8.7 Hz, 1H), 6.12 (d, J=2.2 Hz, 1H), 5.73 (s, 1H), 4.82 (dd, J=10.7, 5.9 Hz, 1H), 4.73 (p, J=7.2 Hz, 1H), 4.61 (dd, J=12.0, 2.2 Hz, 1H), 4.55-4.45 (m, 1H), 2.94-2.85 (m, 2H), 2.68 (dd, J=10.9, 8.1 Hz, 2H), 2.41-2.33 (m, 1H), 2.28 (s, 6H), 1.77-1.66 (m, 1H); MS (ESI) m/z 498 (M+H)⁺.

### Example 200: (2R,4R)-6-chloro-4-hydroxy-N-[3-({(1RS,2SR)-2-[(trifluoromethoxy)methyl]cyclopropane-1-carbonyl}amino)bicyclo[1.1.1]pentan-1-yl]-3,4-dihydro-2H-1-benzopyran-2-carboxamide (Compound 299)

The methodologies described in Example 5 substituting Example 197 for Example 4 and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (400 MHz, DMSO-d₆) δ ppm 8.79 (s, 1H), 8.64 (s, 1H), 7.37 (s, 1H), 7.23-7.16 (m, 1H), 6.88 (d, J=8.7 Hz, 1H), 6.27 (s, 1H), 5.68 (d, J=6.3 Hz, 1H), 4.84-4.74 (m, 1H), 4.58 (d, J=10.2 Hz, 1H), 4.35 (dd, J=10.3, 5.9 Hz, 1H), 4.21-4.12 (m, 1H), 2.22 (s, 6H), 1.77-1.68 (m, 1H), 1.15 (s, 1H), 1.07-0.94 (m, 1H), 0.88 (d, J=5.0 Hz, 1H); MS (APCI⁺) m/z 456 (M−H₂O+H)⁺.

### Example 201: (2R,4R)-6-chloro-4-hydroxy-N-[3-(2-{[cis-3-(trifluoromethoxy)cyclobutyl]oxy}-1,3-thiazol-4-yl)bicyclo[1.1.1]pentan-1-yl]-3,4-dihydro-2H-1-benzopyran-2-carboxamide (Compound 300)

### Example 201A: cis-3-(benzyloxy)cyclobutanol

To a solution of 3-(benzyloxy)cyclobutanone (20 g, 113 mmol) in methanol (200 mL) was added sodium borohydride (4.29 g, 113 mmol) portionwise at −30° C. over 10 minutes. The reaction mixture was stirred at −30° C. for 1 hour, was quenched with ammonium chloride (saturated aqueous, 100 mL) at −20° C., and concentrated in vacuo. The residue was extracted with ethyl acetate (3×1000 mL). The combined organic layers were dried over Na₂SO₄, filtered, and concentrated in vacuo. The residue was combined with another batch and purified by column chromatography on silica gel (10:1 petroleum ether:ethyl acetate) to give the title intermediate (56 g, 283 mmol, 83% yield). ¹H NMR (400 MHz, DMSO-d₆) δ ppm 7.22-7.39 (m, 5H), 5.00 (d, J=6.63 Hz, 1H), 4.33 (s, 1H), 4.30-4.36 (m, 1H), 3.68 (sxt, J=7.10 Hz, 1H), 3.54 (quin, J=7.07 Hz, 1H), 2.51-2.60 (m, 2H), 1.73 (qd, J=8.09, 2.88 Hz, 2H).

### Example 201B: ((cis-3-(trifluoromethoxy)cyclobutoxy)methyl)benzene

The title compound was synthesized using the same procedure as described in Example 13O substituting the product of Example 13N with the product of Example 201A. ¹H NMR (500 MHz, DMSO-d₆) δ ppm 7.24-7.40 (m, 2H), 4.46-4.57 (m, 1H), 4.39 (s, 1H), 3.65-3.79 (m, 1H), 2.69-2.80 (m, 1H), 2.08 (td, J=9.60, 7.57 Hz, 1H).

### Example 201C: cis-3-(trifluoromethoxy)cyclobutanol

To a solution of the product of Example 201B (3.00 g, 11.0 mmol) in methanol (45 mL) was added palladium on carbon (1.17 g, 0.548 mmol) under argon. The reaction mixture was stirred at 50° C. under hydrogen (50 psi) for 12 hours. Then the suspension was filtered through a pad of diatomaceous earth and the pad was washed with methanol (3×200 mL). The combined filtrates were concentrated and purified by column chromatography on silica gel (10:1 petroleum ether:ethyl acetate) to give the title intermediate (0.800 g, 4.61 mmol, 42% yield). ¹H NMR (500 MHz, DMSO-d₆) δ ppm 5.31 (d, J=6.75 Hz, 1H), 5.31 (d, J=6.75 Hz, 1H), 4.36 (quin, J=7.19 Hz, 1H), 3.75 (sxt, J=7.05 Hz, 1H), 1.92-2.08 (m, 2H).

### Example 201D: (2R,4R)-6-chloro-4-hydroxy-N-(3-{1-[cis-3-(trifluoromethoxy)cyclobutyl]-1H-imidazol-4-yl}bicyclo[1.1.1]pentan-1-yl)-3,4-dihydro-2H-1-benzopyran-2-carboxamide

The title compound was synthesized using the same procedures as described in Example 187A through Example 187C substituting the product of Example 119A with the product of Example 201C. ¹H NMR (500 MHz, DMSO-d₆) δ ppm 8.71 (s. 1H), 7.38 (dd, J=2.7, 1.0 Hz, 1H), 7.20 (dd, J=8.7, 2.7 Hz, 1H), 6.89 (d, J=8.7 Hz, 1H), 6.73 (s, 1H), 5.70 (d, J=6.3 Hz, 1H), 4.87-4.77 (m, 2H), 4.69-4.57 (m, 2H), 3.03-2.96 (m, 2H), 2.40-2.33 (m, 3H), 2.24 (s, 6H), 1.74-1.66 (m, 1H).

### Example 202: (2R,4R)-6-chloro-4-hydroxy-N-[3-({4-[cis-3-(trifluoromethoxy)cyclobutyl]-1,3-thiazol-2-yl}oxy)bicyclo[1.1.1]pentan-1-yl]-3,4-dihydro-2H-1-benzopyran-2-carboxamide (Compound 301)

### Example 202A: Tert-Butyl (3-(carbamothioyloxy)bicyclo[1.1.1]pentan-1-yl)carbamate

To a solution of tert-butyl (3-hydroxybicyclo[1.1.1]pentan-1-yl)carbamate (PharmaBlock, 50 mg, 0.25 mmol) and N,N-dimethylpyridin-4-amine (3.1 mg, 0.025 mmol) in tetrahydrofuran (1 mL) at ambient temperature under nitrogen was added di(1H-imidazol-1-yl)methanethione (49 mg, 0.28 mmol) and the reaction mixture stirred for 2 hours. To the reaction mixture was added tetrahydrofuran (1 mL), followed by ammonium hydroxide (0.034 mL, 0.50 mmol) and the reaction mixture stirred at ambient temperature for 3 days. Then water (10 mL) was added and the suspension was extracted with ethyl acetate (3-10 mL). The combined extracts were washed with brine (5 mL), dried over MgSO₄, filtered and concentrated in vacuo. The crude residue was purified by flash chromatography on silica gel (0-100% ethyl acetate in isohexanes) to afford the title intermediate (15 mg, 0.057 mmol, 23% yield). ¹H NMR (500 MHz, DMSO-d₆) δ ppm 8.74 (s, 1H), 8.46 (s, 1H), 7.62 (s, 1H), 2.39 (s, 6H), 1.38 (s, 9H).

### Example 202B: Tert-Butyl (3-((4-((cis)-3-(trifluoromethoxy)cyclobutyl)thiazol-2-yl)ox)bicyclo[1.1.1]pentan-1-yl)carbamate

A solution of Example 193C (14 mg, 0.053 mmol) in ethanol (0.8 mL) was added to a solution of Example 202A (14 mg, 0.053 mmol) and triethylamine (0.011 mL, 0.080 mmol) in ethanol (0.2 mL). The reaction mixture was stirred at 80° C. for 4 days and then concentrated in vacuo. The residue was purified by chromatography on silica gel (0-100% ethyl acetate in heptane) to afford the title intermediate (38 mg, 0.053 mmol, quantitative yield). ¹H NMR (500 MHz, DMSO-d₆) δ ppm 7.71 (s, 1H), 6.81 (s, 1H), 4.78 (p, J=7.5 Hz, 1H), 3.09-2.97 (m, 1H), 2.70-2.60 (m, 2H), 2.38 (s, 6H), 2.36-2.27 (m, 2H), 1.39 (s, 9H); MS (ESI+) m/z 443 (M+Na)⁺.

### Example 202C: 3-((4-((cis)-3-(trifluoromethoxy)cyclobutyl)thiazol-2-yl)oxy)bicyclo[1.1.1]pentan-1-amine

The methodologies described in Example 21B substituting Example 202B for Example 21A gave the title compound. MS (ESI+) m/z 321 (M+H)⁺.

### Example 202D: (2R,4R)-6-chloro-4-hydroxy-N-[3-({4-[cis-3-(trifluoromethoxy)cyclobutyl]-1,3-thiazol-2-yl}oxy)bicyclo[1.1.1]pentan-1-yl]-3,4-dihydro-2H-1-benzopyran-2-carboxamide

The methodologies described in Example 155C substituting Example 202C for Example 155B gave the title compound. ¹H NMR (500 MHz, DMSO-d₆) δ ppm 8.87 (s, 1H), 7.39 (d, J=2.7 Hz, 1H), 7.21 (dd, J=8.8, 2.7 Hz, 1H), 6.89 (d, J=8.7 Hz, 1H), 6.84 (s, 1H), 5.73-5.69 (m, 1H), 4.84-4.78 (m, 2H), 4.67-4.63 (m, 1H), 3.07-3.03 (m, 1H), 2.70-2.64 (m, 2H), 2.53 (s, 6H), 2.39-2.35 (m, 2H), 1.72 (q, J=11.8 Hz, 2H); MS (ESI+) m/z 513 (M−H₂O+H)⁺.

### Example 203: (2R,4R)-6-chloro-4-hydroxy-N-(3-{4-[4-(trifluoromethoxy)phenyl]-1H-pyrazol-1-yl}bicyclo[1.1.1]pentan-1-yl)-3,4-dihydro-2H-1-benzopyran-2-carboxamide (Compound 302)

### Example 203A: 4-(4-(trifluoromethoxy)phenyl)-1-trityl-1H-pyrazole

Potassium carbonate (0.380 g, 2.75 mmol), 4-(4,4,5,5-tetramethyl-[1,3,2]dioxaborolan-2-yl)-1-trityl-1H-pyrazole (0.48 g, 1.10 mmol, ArkPharm), [1,1′-bis(diphenylphosphino)ferrocene]dichloropalladium(II), complex with dichloromethane (90 mg, 0.11 mmol), and 1-bromo-4-(trifluoromethoxy)benzene (345 mg, 1.43 mmol) were combined with 1,2-dimethoxyethane (12 mL) and water (1.2 mL). The vial was degassed by sparging with nitrogen for 2 minutes before sealing with a polytetrafluoroethylene-lined cap. The reaction mixture was stirred at 105° C. for 2 hours, cooled to ambient temperature, and then combined with diatomaceous earth (about 10 grams) and concentrated under reduced pressure to a free flowing powder. The powder was directly purified by reversed-phase flash chromatography [Custom packed YMC TriArt™ C18 Hybrid 20 μm column, 25×150 mm, flow rate 70 mL/minute, 20-100% gradient of acetonitrile in buffer (0.025 M aqueous ammonium bicarbonate, adjusted to pH 10 with ammonium hydroxide)] to give the title compound (252 mg, 0.54 mmol, 49% yield). ¹H NMR (600 MHz, DMSO-d₆) δ ppm 8.11 (d, J=0.8 Hz, 1H), 7.87 (d, J=0.8 Hz, 1H), 7.70-7.65 (m, 2H), 7.41-7.32 (m, 9H), 7.32-7.28 (m, 2H), 7.16-7.08 (m, 6H).

### Example 203B: 4-(4-(trifluoromethoxy)phenyl)-1H-pyrazole

A mixture of trifluoroacetic acid (3.3 mL), methanol (3.3 mL), and dichloromethane (3.3 mL) was added to the product of Example 203A (0.21 g, 0.45 mmol). The resulting solution was stirred at ambient temperature for 1 hour and concentrated under reduced pressure. The residue was taken up in N,N-dimethylformamide (4 mL), filtered through a glass microfiber frit, and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (66 mg, 0.29 mmol, 65% yield); MS (APCI⁺) m/z 229 (M+H)⁺.

### Example 203C: Methyl 3-(4-(4-(trifluoromethoxy)phenyl)-1H-pyrazol-1-yl)bicyclo[1.1.1]pentane-1-carboxylate

A 30 mL vial was charged with iodomesitylene diacetate (211 mg, 0.58 mmol), 3-(methoxycarbonyl)bicyclo[1.1.1]pentane-1-carboxylic acid (197 mg, 1.16 mmol) and toluene (2 mL). The mixture was stirred at 60° C. for 30 minutes. Toluene was then removed under high vacuum. The product of Example 203B (66 mg, 0.29 mmol), tris(2-phenylpyridine)iridium (3.3 mg, 5.0 μmol), and copper(II) acetylacetonate (38 mg, 0.145 mmol) were added followed by dioxane (5 mL). The vial was degassed by sparging with nitrogen for 3 minutes before sealing with a polytetrafluoroethylene-lined cap. The reaction was stirred and irradiated using 2 lamps: a 40W Kessil PR160 390 nm photoredox lamp, and an 18W 450 nm HepatoChem blue LED photoredox lamp. Both lamps were placed 3 cm away from the reaction vial set inside a continuously running tap water bath. The reaction temperature was measured to be 12° C. and maintained at that temperature for the duration of the reaction. After 12 hours, the reaction mixture was quenched by exposing to air and partitioned between water (50 mL) and dichloromethane (2×50 mL). The organic layers were combined and dried over sodium sulfate and concentrated under reduced pressure. The residue was taken up in methanol (15 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (43 mg, 0.12 mmol, 42% yield). ¹H NMR (400 MHz, DMSO-d₆) δ ppm 8.35 (d, J=0.8 Hz, 1H), 7.99 (d, J=0.8 Hz, 1H), 7.75-7.68 (m, 2H), 7.39-7.32 (m, 2H), 3.68 (s, 3H), 2.53 (s, 6H); MS (APCI⁺) m/z 353 (M+H)⁺.

### Example 203D: 3-(4-(4-(trifluoromethoxy)phenyl)-1H-pyrazol-1-yl)bicyclo[1.1.1]pentane-1-carboxylic Acid

The reaction and purification conditions described in Example 110B substituting the product of Example 203C for the product of Example 110A gave the title compound. MS (APCI⁺) m/z 339 (M+H)⁺.

### Example 203E: 2-(trimethylsilyl)ethyl (3-(4-(4-(trifluoromethoxy)phenyl)-1H-pyrazol-1-yl)bicyclo[1.1.1]pentan-1-yl)carbamate

The reaction and purification conditions described in Example 125C substituting the product of Example 203D for the product of Example 125B gave the title compound. MS (APCI⁺) m/z 454 (M+H)⁺.

### Example 203F: (2R,4R)-6-chloro-4-hydroxy-N-(3-{4-[4-(trifluoromethoxy)phenyl]-1H-pyrazol-1-yl}bicyclo[1.1.1]pentan-1-yl)-3,4-dihydro-2H-1-benzopyran-2-carboxamide

The reaction and purification conditions described in Example 1C substituting the product of Example 203E for the product of Example 1A, and the product of Example 3B for the product of Example 1B gave the title compound. ¹H NMR (400 MHz, DMSO-d₆) δ ppm 8.91 (s, 1H), 8.33 (s, 1H), 7.98 (s, 1H), 7.77-7.69 (m, 2H), 7.39 (dd, J=2.7, 0.9 Hz, 1H), 7.35 (d, J=8.3 Hz, 2H), 7.22 (dd, J=8.7, 2.7 Hz, 1H), 6.91 (d, J=8.7 Hz, 1H), 5.73 (br s, 1H), 4.83 (dd, J=10.7, 5.8 Hz, 1H), 4.66 (dd, J=11.9, 2.3 Hz, 1H), 2.55 (s, 6H), 2.39 (ddd, J=12.9, 5.9, 2.4 Hz, 1H), 1.74 (td, J=12.4, 10.8 Hz, 1H); MS (APCI⁺) m/z 520 (M+H)⁺.

### Example 204: (2R,4R)-6-chloro-N-[trans-4-{3-[5-(difluoromethyl)pyrazin-2-yl]-2-oxoimidazolidin-1-yl}cyclohexyl]-4-hydroxy-3,4-dihydro-2H-1-benzopyran-2-carboxamide (Compound 303)

The reaction and purification conditions described in Example 136D substituting 1-(trans-4-aminocyclohexyl)-3-[5-(difluoromethyl)-2-pyrazinyl]-2-imidazolidinone (prepared as described in International Patent Publication WO2019/090081 A1) for the product of Example 136C gave the title compound. ¹H NMR (500 MHz, DMSO-d₆) δ 8.50-8.45 (m, 1H), 8.31 (dd, J=8.9, 0.8 Hz, 1H), 7.94-7.87 (m, 2H), 7.38 (dd, J=2.7, 1.0 Hz, 1H), 7.20 (ddd, J=8.7, 2.7, 0.7 Hz, 1H), 7.04 (t, J=55.6 Hz, 1H), 6.89 (d, J=8.7 Hz, 1H), 5.69 (br s, 1H), 4.81 (dd, J=10.7, 5.9 Hz, 1H), 4.62 (dd, J=12.0, 2.3 Hz, 1H), 3.95 (dd, J=9.0, 7.1 Hz, 2H), 3.72-3.59 (m, 2H), 3.50-3.45 (m, 2H), 2.35 (ddd, J=12.9, 5.9, 2.3 Hz, 1H), 1.89-1.82 (m, 2H), 1.78-1.68 (m, 2H), 1.62 (qt, J=12.2, 2.7 Hz, 2H), 1.54-1.39 (m, 2H); MS (APCI⁺) m/z 522 (M+H)⁺.

### Example 205: (2R,4R)-6-chloro-N-{(1R,2S,4R,5S)-5-[4-(3,4-difluorophenyl)-1H-imidazol-1-yl]bicyclo[2.2.1]heptan-2-yl}-4-hydroxy-3,4-dihydro-2H-1-benzopyran-2-carboxamide (Compound 304)

### Example 205A: Benzyl ((1R,2S,4R,5S)-5-aminobicyclo[2.2.1]heptan-2-yl)carbamate

The product of Example 111D was purified via chiral separation to give 2 enantiomers. Chiral SFC using Column: (S,S)-Whelk®-O1, 250×30 mm, 10 um, Mobile phase: A: CO₂, B: ethanol (0.1% NH₃), Gradient: 30% B, flow rate: 58 g/minute; column temperature: 40° C.; system back pressure: 100 bar gave the title intermediate as the later eluting isomer. ¹H NMR (400 MHz, methanol-d₄) δ ppm 7.44-7.20 (m, 5H), 5.05 (s, 2H), 3.37 (br dd, J=2.9, 7.8 Hz, 1H), 2.81 (br d, J=4.4 Hz, 1H), 2.20 (br d, J=3.4 Hz, 1H), 2.10 (br d, J=3.9 Hz, 1H), 1.74-1.60 (m, 2H), 1.57-1.50 (m, 1H), 1.46-1.40 (m, 1H), 1.32 (td, J=4.0, 13.4 Hz, 1H), 1.22-1.12 (m, 1H); MS (ESI⁺) m/z 261 (M+H)⁺.

### Example 205B: Benzyl ((1R,2S,4R,5S)-5-(4-(3,4-difluorophenyl)-1H-imidazol-1-yl)bicyclo[2.2.1]heptan-2-yl)carbamate

The methodologies described in Example 49A substituting the product of Example 205A for tert-butyl (3-aminobicyclo[1.1.1]pentan-1-yl)carbamate, increasing the reaction time from 4.5 hours to 3 days, and eliminating purification by HPLC gave the title intermediate, which was carried forward without purification. MS (APCI) m/z 424 (M+H)⁺.

### Example 205C: (1R,2S,4R,5S)-5-(4-(3,4-difluorophenyl)-1H-imidazol-1-yl)bicyclo[2.2.1]heptan-2-amine

To solution of the product of Example 205B (0.160 g, 0.377 mmol) in dichloromethane (0.75 mL) was added trifluoroacetic acid (2.18 mL, 28.2 mmol). This mixture was allowed to stir at 70° C. for 4 hours and then was concentrated in vacuo to provide the title intermediate (0.109 g, 0.377 mmol, quantitative yield). MS (ESI+) m/z 290 (M+H)⁺.

### Example 205D: (2R)-6-chloro-N-{(1R,2S,4R,5S)-5-[4-(3,4-difluorophenyl)-1H-imidazol-1-yl]bicyclo[2.2.1]heptan-2-yl}-4-oxo-3,4-dihydro-2H-1-benzopyran-2-carboxamide

The methodologies described in Example 30D substituting the product of Example 1B for 3-(2-(4-chloro-3-fluorophenoxy)acetamido)bicyclo[1.1.1]pentane-1-carboxylic acid and substituting Example 205C for Example 30C gave the title intermediate. MS (APCI⁺) m/z 498 (M+H)⁺.

### Example 205E: (2R,4R)-6-chloro-N-{(1R,2S,4R,5S)-5-[4-(3,4-difluorophenyl)-1H-imidazol-1-yl]bicyclo[2.2.1]heptan-2-yl}-4-hydroxy-3,4-dihydro-2H-1-benzopyran-2-carboxamide

The methodologies described in Example 5 substituting Example 205D for Example 4 and purifying by preparative HPLC (Waters XBridgem C18 5 μm OBD column, 30×100 mm, flow rate 40 mL/minute, 5-100% gradient of acetonitrile in 0.1% trifluoroacetic acid/water) gave the title compound. ¹H NMR (400 MHz, DMSO-d₆) δ ppm 8.94 (s, 1H), 8.25 (s, 1H), 7.99 (d, J=6.8 Hz, 1H), 7.89 (t, J=9.6 Hz, 1H), 7.66 (s, 1H), 7.59 (q, J=9.1 Hz, 1H), 7.39 (d, J=2.9 Hz, 1H), 7.23-7.18 (m, 1H), 7.09 (s, 1H), 6.89 (d, J=8.7 Hz, 1H), 4.82 (dd, J=10.5, 5.8 Hz, 1H), 4.63 (dd, J=11.8, 2.4 Hz, 1H), 4.34 (m, 1H), 2.63 (m, 1H), 2.33 (m, 3H), 2.04-1.94 (m, 1H), 1.85-1.74 (m, 1H), 1.63-1.52 (m, 3H), 1.17-1.07 (m, 1H); MS (APCI⁺) m/z 500 (M+H)⁺.

### Example 206: (2R,4R)-6-chloro-N-{3-[2-(4-chloro-3-fluorophenyl)-1,3-oxazol-5-yl]bicyclo[1.1.1]pentan-1-yl}-4-hydroxy-3,4-dihydro-2H-1-benzopyran-2-carboxamide (Compound 305)

### Example 206A: 1-(3-aminobicyclo[1.1.1]pentan-1-yl)ethanone

To a solution of Example 173B (220 mg, 0.977 mmol) in dichloromethane (4 mL) was added HCl (4 N in dioxane, 4 mL, 16 mmol) and the reaction mixture stirred at ambient temperature overnight. Then the solvent was removed under reduced pressure to give the title intermediate as an HCl salt (0.160 g, 0.980 mmol, quantitative yield). ¹H NMR (500 MHz, DMSO-d₆) δ ppm 8.81 (s, 3H), 2.21 (s, 6H), 2.14 (s, 3H).

### Example 206B: Benzyl (3-acetylbicyclo[1.1.1]pentan-1-yl)carbamate

To a solution of Example 206A (160 mg, 1.28 mmol) and NaOH (102 mg, 2.56 mmol) in tetrahydrofuran (5 mL) and water (5 mL) was added benzyl chloroformate (CbZ-Cl, 0.201 mL, 1.41 mmol) dropwise. The resulting solution was stirred at ambient temperature overnight. Then volatiles were removed under reduced pressure to give the title intermediate (0.279 g, 0.947 mmol, 74% yield). ¹H NMR (500 MHz, DMSO-d₆) δ ppm 7.38-7.32 (m, 5H), 4.98 (s, 2H), 4.49 (s, 1H), 2.12 (d, J=4.5 Hz, 6H), 2.09 (s, 3H).

### Example 206C: Benzyl (3-(2-bromoacetyl)bicyclo[1.1.1]pentan-1-yl)carbamate

To a solution of Example 206B (0.279 g, 0.947 mmol) in tetrahydrofuran (3 mL) at 0° C. was added phenyltrimethylammonium tribromide (0.356 g, 0.947 mmol) portionwise. The resulting solution was stirred at ambient temperature for 2 hours. The reaction mixture was filtered, washing with tetrahydrofuran (2.5 mL), and the filtrate was concentrated in vacuo. The residue was purified by chromatography on silica gel (0-50% ethyl acetate in isohexane) to afford the title intermediate (211 mg, 0.499 mmol, 53% yield). ¹H NMR (500 MHz, DMSO-d₆) δ ppm 8.09 (s, 1H), 7.39-7.30 (m, 5H), 5.01 (s, 2H), 4.47 (s, 2H), 2.23 (s, 6H).

### Example 206D: Benzyl (3-(2-aminoacetyl)bicyclo[1.1.1]pentan-1-yl)carbamate Hydrochloride

The methodologies described in Example 193D substituting Example 206C for Example 193C gave the title intermediate. ¹H NMR (500 MHz, DMSO-d₆) δ ppm 8.89 (s, 1H), 8.14 (s, 3H), 7.40-7.34 (m, 5H), 5.01 (s, 2H), 4.02 (s, 2H), 2.24 (s, 6H).

### Example 206E: Benzyl (3-(2-(4-chloro-3-fluorobenzamido)acetyl)bicyclo[1.1.1]pentan-1-yl)carbamate

The methodologies described in Example 155A substituting Example 206D for 2-amino-1-(4-chlorophenyl)ethanone hydrochloride, substituting 4-chloro-3-fluorobenzoic acid for 3-((tert-butoxycarbonyl)amino)bicyclo[1.1.1]pentane-1-carboxylic acid, and purifying by column chromatography on silica gel (0-100% ethyl acetate in hexanes) gave the title intermediate. ¹H NMR (500 MHz, DMSO-d₆) δ ppm 8.93 (t, J=5.6 Hz, 1H), 8.49 (s, 1H), 7.88-7.85 (m, 2H), 7.77-7.70 (m, 5H), 7.62-7.60 (m, 1H), 5.01 (s, 2H), 4.20 (d, J=5.1 Hz, 2H), 1.99 (s, 6H).

### Example 206F: 3-(2-(4-chloro-3-fluorophenyl)oxazol-5-yl)bicyclo[1.1.1]pentan-1-amine

The methodologies described in Example 155B substituting Example 206E for Example 155A gave the title intermediate. ¹H NMR (500 MHz, DMSO-d₆) δ ppm 8.40 (s, 2H), 7.86 (dd, J=10.0, 1.7 Hz, 1H), 7.78-7.73 (m, 2H), 7.09 (s, 1H), 2.07 (s, 6H).

### Example 206G: (2R,4R)-6-chloro-N-{3-[2-(4-chloro-3-fluorophenyl)-1,3-oxazol-5-yl]bicyclo[1.1.1]pentan-1-yl}-4-hydroxy-3,4-dihydro-2H-1-benzopyran-2-carboxamide

The methodologies described in Example 155C substituting Example 206F for Example 155B gave the title compound. ¹H NMR (500 MHz, DMSO-d₆) δ ppm 8.85 (s, 1H), 7.90 (dd, J=9.9, 1.9 Hz, 1H), 7.81-7.75 (m, 2H), 7.40 (dd, J=2.8, 1.0 Hz, 1H), 7.22 (dd, J=8.7, 2.7 Hz, 1H), 7.19 (s, 1H), 6.90 (d, J=8.7 Hz, 1H), 5.95-5.50 (m, 1H), 4.84-4.80 (m, 1H), 4.63 (dd, J=12.0, 2.3 Hz, 1H), 2.43 (s, 6H), 1.76-1.69 (m, 1H); MS (ESI+) m/z 489 (M+H)⁺.

### Example 207: (2S,4R)-6-chloro-N-(3-{4-[3-fluoro-4-(trifluoromethoxy)phenyl]-1H-pyrazol-1-yl}bicyclo[1.1.1]pentan-1-yl)-4-hydroxy-3,4-dihydro-2H-1-benzopyran-2-carboxamide (Compound 306)

### Example 207A: Methyl 3-(4-bromo-1H-pyrazol-1-yl)bicyclo[1.1.1]pentane-1-carboxylate

The reaction and purification conditions described in Example 203C substituting 4-bromo-1H-pyrazole for the product of Example 203B gave the title compound. MS (APCI⁺) m/z 271, 273 (M+H)⁺.

### Example 207B: 3-(4-bromo-1H-pyrazol-1-yl)bicyclo[1.1.1]pentane-1-carboxylic Acid

The reaction and purification conditions described in Example 110B substituting the product of Example 207A for the product of Example 110A gave the title compound. MS (APCI⁺) m/z 257, 259 (M+H)⁺.

### Example 207C: Tert-Butyl (3-(4-bromo-1H-pyrazol-1-yl)bicyclo[1.1.1]pentan-1-yl)carbamate

A mixture of the product of Example 207B (100 mg, 0.39 mmol), N,N-diisopropylethylamine (0.136 mL, 0.78 mmol) and tert-butanol (2 mL) was stirred at ambient temperature. Diphenylphosphoryl azide (0.109 mL, 0.506 mmol) was added. The mixture was stirred at 58° C. for 10 hours, cooled, and concentrated under reduced pressure. The residue was taken up in methanol (5 mL), filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (115 mg, 0.35 mmol, 90% yield). MS (ESI⁺) m/z 328, 330 (M+H)⁺.

### Example 207D: Tert-Butyl (3-(4-(3-fluoro-4-(trifluoromethoxy)phenyl)-1H-pyrazol-1-yl)bicyclo[1.1.1]pentan-1-yl)carbamate

Potassium carbonate (105 mg, 0.762 mmol), tris(dibenzylideneacetone)dipalladium(0) (42 mg, 0.046 mmol), 1,3,5,7-tetramethyl-6-phenyl-2,4,8-trioxa-6-phosphaadamantane (27 mg, 0.092 mmol), 3-fluoro-4-(trifluoromethoxy)phenylboronic acid (82 mg, 0.366 mmol, Combi-Blocks), and the product of Example 207C (100 mg, 0.305 mmol) were combined with 1,2-dimethoxyethane (5 mL) and water (0.5 mL) in a 20 mL vial. The vial was sealed and degassed three times with a nitrogen backflush each time. It was then heated at 58° C. for 18 hours. The reaction mixture was cooled to ambient temperature, and then partitioned between dichloromethane (2×30 mL) and aqueous sodium carbonate (1.0 M, 30 mL). The organic layers were combined and dried over anhydrous sodium sulfate and concentrated under reduced pressure. The residue was purified by preparative HPLC [YMC TriArt™ C18 Hybrid 5 μm column, 50×100 mm, flow rate 140 mL/minute, 15-100% gradient of acetonitrile in buffer (0.025 M aqueous ammonium bicarbonate, adjusted to pH 10 with ammonium hydroxide)] to give the title compound (0.12 g, 0.28 mmol, 92% yield). MS (APCI⁺) m/z 428 (M+H)⁺.

### Example 207E: (2S,4R)-6-chloro-N-(3-{4-[3-fluoro-4-(trifluoromethoxy)phenyl]-1H-pyrazol-1-yl}bicyclo[1.1.1]pentan-1-yl)-4-hydroxy-3,4-dihydro-2H-1-benzopyran-2-carboxamide

The reaction and purification conditions described in Example 1C substituting the product of Example 207D for the product of Example 1A, and the product of Example 73B for the product of Example 1B gave the title compound. ¹H NMR (600 MHz, DMSO-d₆) δ ppm 8.97 (s, 1H), 8.42 (d, J=0.8 Hz, 1H), 8.05 (d, J=0.8 Hz, 1H), 7.82-7.76 (m, 1H), 7.59-7.50 (m, 2H), 7.33 (d, J=2.6 Hz, 1H), 7.27 (dd, J=8.8, 2.7 Hz, 1H), 6.95 (d, J=8.7 Hz, 1H), 5.64 (d, J=4.6 Hz, 1H), 4.63-4.58 (m, 2H), 2.55 (s, 6H), 2.13 (ddd, J=13.9, 3.8, 2.8 Hz, 1H), 1.93 (ddd, J=13.8, 11.0, 3.6 Hz, 1H); MS (APCI⁺) m/z 538 (M+H)⁺.

### Example 208: (2R,4R)-6-chloro-N-{3-[4-(4-chlorophenyl)-2-oxopyrrolidin-1-yl]bicyclo[1.1.1]pentan-1-yl}-4-hydroxy-3,4-dihydro-2H-1-benzopyran-2-carboxamide (Compound 307)

### Example 208A: Methyl 3-(4-(4-chlorophenyl)-2-oxopyrrolidin-1-yl)bicyclo[1.1.1]pentane-1-carboxylate

The reaction and purification conditions described in Example 203C substituting 4-(4-chlorophenyl)pyrrolidin-2-one (J-W Pharmlab) for the product of Example 203B gave the title compound. MS (APCI⁺) m/z 320 (M+H)⁺.

### Example 208B: 3-(4-(4-chlorophenyl)-2-oxopyrrolidin-1-yl)bicyclo[1.1.1]pentane-1-carboxylic Acid

The reaction and purification conditions described in Example 110B substituting the product of Example 208A for the product of Example 110A gave the title compound. (APCI⁺) m/z 347 (M+CH₃CN+H)⁺.

### Example 208C: 2-(trimethylsilyl)ethyl (3-(4-(4-chlorophenyl)-2-oxopyrrolidin-1-yl)bicyclo[1.1.1]pentan-1-yl)carbamate

The reaction and purification conditions described in Example 125C substituting the product of Example 208B for the product of Example 125B gave the title compound. (APCI⁺) m/z 421 (M+H)⁺.

### Example 208D: (2R,4R)-6-chloro-N-{3-[4-(4-chlorophenyl)-2-oxopyrrolidin-1-yl]bicyclo[1.1.1]pentan-1-yl}-4-hydroxy-3,4-dihydro-2H-1-benzopyran-2-carboxamide

The reaction and purification conditions described in Example 186B substituting the product of Example 208C for the product of Example 186A gave the title compound. ¹H NMR (400 MHz, DMSO-d₆) δ ppm (s, 1H), 7.43-7.30 (m, 5H), 7.19 (dd, J=8.7, 2.7 Hz, 1H), 6.88 (d, J=8.7 Hz, 1H), 4.80 (dd, J=10.7, 5.8 Hz, 1H), 4.60 (dd, J=11.9, 2.3 Hz, 1H), 3.71 (dd, J=9.4, 8.1 Hz, 1H), 3.63-3.50 (m, 1H), 3.27 (dd, J=9.3, 7.7 Hz, 1H), 2.63 (dd, J=16.5, 8.71 Hz, 1H), 2.46-2.30 (m, 2H), 2.33 (s, 6H), 1.69 (td, J=12.6, 10.8 Hz, 1H); MS (APCI⁺) m/z 487 (M+H)⁺.

### Example 209: (2R,4R)-6-chloro-4-hydroxy-N-(3-{4-[5-(trifluoromethoxy)pyridin-2-yl]-1H-pyrazol-1-yl}bicyclo[1.1.1]pentan-1-yl)-3,4-dihydro-2H-1-benzopyran-2-carboxamide (Compound 308)

The reaction and purification conditions described in Examples 203A through 203F substituting 2-bromo-5-(trifluoromethoxy)pyridine (Ark Pharm) for 1-bromo-4-(trifluoromethoxy)benzene gave the title compound ¹H NMR (500 MHz, DMSO-d₆) δ ppm 8.92 (s, 1H), 8.59-8.55 (m, 1H), 8.44 (d, J=0.8 Hz, 1H), 8.09 (d, J=0.8 Hz, 1H), 7.90-7.86 (m, 1H), 7.86-7.83 (m, 1H), 7.39 (dd, J=2.7, 1.0 Hz, 1H), 7.21 (ddd, J=8.7, 2.7, 0.7 Hz, 1H), 6.90 (d, J=8.7 Hz, 1H), 5.75 (br s, 1H), 4.83 (dd, J=10.6, 5.9 Hz, 1H), 4.65 (dd, J=11.9, 2.3 Hz, 1H), 2.56 (s, 6H), 2.43-2.35 (m, 1H), 1.73 (ddd, J=13.0, 12.0, 10.7 Hz, 1H); MS (ESI⁺) m/z 521 (M+H)⁺.

### Example 210: (2S,4R)-6-chloro-4-hydroxy-N-[trans-4-2-oxo-3-[6-(trifluoromethyl)pyridin-3-yl]imidazolidin-1-yl)cyclohexyl]-3,4-dihydro-2H-1-benzopyran-2-carboxamide (Compound 309)

### Example 210A: Benzyl (trans-4-(2-oxo-3-(6-(trifluoromethyl)pyridin-3-yl)imidazolidin-1-yl)cyclohexyl)carbamate

5-Iodo-2-(trifluoromethyl)pyridine (276 mg, 1.01 mmol Ark Pharm), bis(tri-tert-butylphosphine)palladium(0) (24 mg, 0.047 mmol), the product of Example 37C (247 mg, 0.778 mmol) and cesium carbonate (507 mg, 1.556 mmol) were suspended in dioxane (5 mL) in a 20 mL vial. The vial was degassed by sparging with nitrogen for 2 minutes before sealing with a polytetrafluoroethylene-lined cap. The reaction was stirred at 58° C. for 18 hours, cooled to ambient temperature, and more bis(tri-tert-butylphosphine)palladium(0) (12 mg, 0.043 mmol) was added. The vial was resealed and heated at 100° C. for 4 hours, and then cooled. The resulting reaction mixture was partitioned between ethyl acetate (2×50 mL) and brine (50 mL). The organic layers were combined and dried over sodium sulfate and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (66 mg, 0.143 mmol, 18% yield). MS (APCI⁺) m/z 463 (M+H)⁺.

### Example 210B: (2S,4R)-6-chloro-4-hydroxy-N-[trans-4-{2-oxo-3-[6-(trifluoromethyl)pyridin-3-yl]imidazolidin-1-yl}cyclohexyl]-3,4-dihydro-2H-1-benzopyran-2-carboxamide

The reaction and purification conditions described in Example 1C substituting the product of Example 210A for the product of Example 1A, the product of Example 73B for the product of Example 1B, and also raising the reaction temperature for the first step from ambient temperature in trifluoroacetic acid to 70° C. in trifluoroacetic acid gave the title compound. ¹H NMR (400 MHz, DMSO-d₆) δ ppm 8.95 (d, J=2.6 Hz, 1H), 8.17 (dd, J=8.9, 2.6 Hz, 1H), 8.00 (d, J=8.2 Hz, 1H), 7.82 (d, J=8.8 Hz, 1H), 7.31 (d, J=2.7 Hz, 1H), 7.24 (dd, J=8.7, 2.7 Hz, 1H), 6.94 (d, J=8.7 Hz, 1H), 5.64 (br s, 1H), 4.58 (dd, J=10.7, 2.7 Hz, 2H), 3.89 (dd, J=9.3, 6.5 Hz, 2H), 3.71-3.59 (m, 2H), 3.56-3.48 (m, 2H), 2.14-2.04 (m, 1H), 1.97-1.79 (m, 3H), 1.75-1.54 (m, 4H), 1.52-1.37 (m, 2H); MS (APCI⁺) m/z 539 (M+H)⁺.

### Example 211: (2R,4R)-6-chloro-4-hydroxy-N-[trans-4-2-oxo-3-[6-(trifluoromethyl)pyridin-3-yl]imidazolidin-1-yl)cyclohexyl]-3,4-dihydro-2H-1-benzopyran-2-carboxamide (Compound 310)

The reaction and purification conditions described in Example 186B substituting the product of Example 210A for the product of Example 186A, and also raising the reaction temperature for the first step from ambient temperature in trifluoroacetic acid to 70° C. in trifluoroacetic acid gave the title compound. ¹H NMR (400 MHz, DMSO-d₆) δ ppm 8.95 (d, J=2.6 Hz, 1H), 8.17 (dd, J=8.9, 2.6 Hz, 1H), 7.94 (d, J=8.2 Hz, 1H), 7.83 (d, J=8.7 Hz, 1H), 7.38 (dd, J=2.6, 1.0 Hz, 1H), 7.19 (dd, J=8.5, 2.7 Hz, 1H), 6.89 (d, J=8.7 Hz, 1H), 5.72 (br s, 1H), 4.81 (dd, J=10.7, 6.0 Hz, 1H), 4.62 (dd, J=11.9, 2.3 Hz, 1H), 3.89 (dd, J=9.3, 6.7 Hz, 2H), 3.71-3.58 (m, 2H), 3.54-3.49 (m, 2H), 2.35 (ddd, J=13.0, 5.9, 2.3 Hz, 1H), 1.85 (s, 2H), 1.78-1.55 (m, 5H), 1.53-1.39 (m, 2H); MS (APCI⁺) m/z 539 (M+H)⁺.

### Example 212: (2R,4R)-6-chloro-N-{3-[1-(4-chloro-3-fluorophenyl)-1H-1,2,3-triazol-4-yl]bicyclo[1.1.1]pentan-1-yl}-4-hydroxy-3,4-dihydro-2H-1-benzopyran-2-carboxamide (Compound 311)

### Example 212A: Tert-Butyl (3-(methoxy(methyl)carbamoyl)bicyclo[1.1.1]pentan-1-yl)carbamate

To a solution of N,O-dimethylhydroxylamine hydrochloride (3.86 g, 39.6 mmol) and 3-((tert-butoxycarbonyl)amino)bicyclo[1.1.1]pentane-1-carboxylic acid (6.0 g, 26.4 mmol) in dichloromethane (100 mL) stirred at 0° C. was added N,N-diisopropylethylamine (18.44 mL, 106 mmol) and 1-[bis(dimethylamino)methylene]-1H-1,2,3-triazolo[4,5-b]pyridinium 3-oxid hexafluorophosphate (15.06 g, 39.6 mmol) in sequential order. The ice bath was removed and the reaction mixture was left stirring at ambient temperature for 3 hours. More dichloromethane (100 mL) was added. The resulting solution was washed with aqueous HCl (1.0 M, 100 mL), saturated aqueous sodium bicarbonate (2×100 mL) and brine (100 mL) in sequential order. The organic phase was dried over sodium sulfate, filtered, and concentrated under reduced pressure. The residue was purified by flash chromatography (silica gel, 0-50% ethyl acetate in isohexane) to give the title compound (6.27 g, 21.11 mmol, 80% yield). ¹H NMR (500 MHz, DMSO-d₆) δ ppm 7.59 (s, 1H), 3.63 (s, 3H), 2.69 (s, 3H), 2.14 (s, 6H), 1.37 (s, 9H).

### Example 212B: Tert-Butyl (3-formylbicyclo[1.1.1]pentan-1-yl)carbamate

The product of example 212A (1.00 g, 3.70 mmol) was dissolved in anhydrous tetrahydrofuran (30 mL) under a nitrogen atmosphere. The solution was cooled to −78° C. and diisobutylaluminum hydride (1.0 M in hexanes, 8.14 mL) was slowly added. The reaction mixture was stirred at −78° C. for 1 hour. Methanol (0.3 mL) was added, and the reaction was stirred at −78° C. for 10 minutes. Aqueous HCl (1.0 M, 50 mL) and ethyl acetate (50 mL) were added, and the dry-ice bath was removed. The mixture was stirred vigorously while warming up to ambient temperature, and the stirring was continued for 2.5 hours. Phases were separated, and the aqueous phase was extracted with ethyl acetate (2×50 mL). The organic layers were combined, dried over sodium sulfate, filtered and concentrated under reduced pressure to give the title compound (780 mg, 3.51 mmol, 95%). ¹H NMR (500 MHz, DMSO-d₆) δ ppm 9.59 (s, 1H), 7.64 (s, 1H), 2.12 (s, 6H), 1.38 (s, 9H).

### Example 212C: Tert-Butyl (3-ethynylbicyclo[1.1.1]pentan-1-yl)carbamate

The product of Example 212B (780 mg, 3.51 mmol) was dissolved in methanol (15 mL), and potassium carbonate (2.91 g, 21.05 mmol) was added. After stirring at ambient temperature for 5 minutes, dimethyl (1-diazo-2-oxopropyl)phosphonate (2.53 mL, 10.52 mmol, Manchester Organics) was slowly added and the resulting mixture was stirred for 16 hours, and then concentrated under reduced pressure. The residue was partitioned between dichloromethane (3-20 mL) and water (20 mL). The organic layers were combined, dried over sodium sulfate and concentrated under reduced pressure. The residue was purified by flash chromatography (silica gel, 0-30% ethyl acetate in isohexane) to give the title compound (624 mg, 2.95 mmol, 84% yield). ¹H NMR (500 MHz, DMSO-d₆) δ ppm 7.60 (s, 1H), 3.09 (s, 1H), 2.13 (s, 6H), 1.36 (s, 9H).

### Example 212D: Tert-Butyl (3-(I-(4-chloro-3-fluorophenyl)-1H-1,2,3-triazol-4-yl)bicyclo[1.1.1]pentan-1-yl)carbamate

The product of Example 212C (116 mg, 0.56 mmol), copper sulfate (1.0 mg, 0.006 mmol), tert-butanol (6 mL) and water (2 mL) were combined in a sealed tube. 4-Azido-1-chloro-2-fluorobenzene (103 mg, 0.60 mmol, Enamine), benzoic acid (6.8 mg, 0.056 mmol), and sodium ascorbate (2.0 mg, 0.010 mmol) were added. The tube was flushed with nitrogen, sealed and stirred at 80° C. for 2 days. The mixture was cooled to ambient temperature and then poured into ice water (25 mL), and then extracted with ethyl acetate (3×25 mL). The organic layers were combined, washed with brine (25 mL), dried over magnesium sulfate, filtered, and concentrated under reduced pressure to give the title compound that was used without further purification (275 mg (about 77% purity), 0.56 mmol, 100% yield). ¹H NMR (500 MHz, DMSO-d₆) δ ppm 8.74 (s, 1H), 8.08-8.01 (m, 1H), 7.88-7.78 (m, 2H), 2.25 (s, 6H), 1.39 (s, 9H); MS (ESI⁺) m/z 379 (M+H)⁺.

### Example 212E: (2R,4R)-6-chloro-N-{3-[I-(4-chloro-3-fluorophenyl)-1H-1,2,3-triazol-4-yl]bicyclo[1.1.1]pentan-1-yl}-4-hydroxy-3,4-dihydro-2H-1-benzopyran-2-carboxamide

The reaction and purification conditions described in Example 3C substituting the product of Example 212D for the product of Example 3A gave the title compound. ¹H NMR (400 MHz, DMSO-d₆) δ ppm 8.82-8.75 (m, 2H), 8.09-8.02 (m, 1H), 7.88-7.79 (m, 2H), 7.40 (d, J=2.7, 1.0 Hz, 1H), 7.22 (dd, J=8.7, 2.7 Hz, 1H), 6.91 (d, J=8.7 Hz, 1H), 5.72 (d, J=5.4 Hz, 1H), 4.88-4.79 (m, 1H), 4.63 (dd, J=12.0, 2.3 Hz, 1H), 2.41 (s, 7H), 1.79-1.65 (m, 1H); MS (ESI⁺) m/z 489 (M+H)⁺.

### Example 213: (2R,4R)-6-chloro-N-(3-{4-[3-fluoro-4-(trifluoromethoxy)phenyl]-1H-pyrazol-1-yl}bicyclo[1.1.1]pentan-1-yl)-4-hydroxy-3,4-dihydro-2H-1-benzopyran-2-carboxamide (Compound 312)

The reaction and purification conditions described in Example 186B substituting the product of Example 207D for the product of Example 186A gave the title compound. ¹H NMR (400 MHz, DMSO-d₆) δ ppm 8.92 (s, 1H), 8.41 (d, J=0.8 Hz, 1H), 8.05 (d, J=0.8 Hz, 1H), 7.82-7.74 (m, 1H), 7.60-7.49 (m, 2H), 7.38 (dt, J=10.6, 9.3 Hz, 1H), 7.12 (ddd, J=12.6, 6.7, 3.0 Hz, 1H), 6.87-6.78 (m, 1H), 4.50 (s, 2H), 2.54 (s, 6H); MS (APCI⁺) m/z 538 (M+H)⁺.

### Example 214: (2R,4R)-6-chloro-4-hydroxy-N-[(1RS,2SR,4RS,5SR)-5-({[5-(trifluoromethyl)pyridin-2-yl]methyl}carbamoyl)-7-oxabicyclo[2.2.1]heptan-2-yl]-3,4-dihydro-2H-1-benzopyran-2-carboxamide (Compound 313)

### Example 214A: rac-(1R,2S,4R,5S)-5-amino-N-((5-(trifluormethyl)pyridin-2-yl)methyl)-7-oxabicyclo[2.2.1]heptane-2-carboxamide Trifluoroacetic Acid

To a solution of the product of Example 86D (50 mg, 0.194 mmol), (5-(trifluoromethyl)pyridin-2-yl)methanamine, hydrochloric acid (47.5 mg, 0.223 mmol) and N-ethyl-N-isopropylpropan-2-amine (0.170 mL, 0.972 mmol) in N,N-dimethylformamide (2.0 mL) was added 2-(3H-[1,2,3]triazolo[4,5-b]pyridin-3-yl)-1,1,3,3-tetramethylisouronium hexafluorophosphate(V) (92 mg, 0.243 mmol) and the mixture was stirred at ambient temperature for 1 hour. N,N-Dimethylformamide was removed under high vacuum and the residue was suspended in methanol (5 mL) and treated with 4 N hydrogen chloride in dioxane (0.486 mL, 1.943 mmol) for 30 minutes at 50° C. Solvent and excess HCl were removed under vacuum and the residue was purified by HPLC (Phenomenex® Luna® C18(2) 10 μm 100 Å AXIA™ column (250 mm×50 mm). A 15-70% gradient of acetonitrile (A) and 0.1% trifluoroacetic acid in water (B) was used over 25 minutes, at a flow rate of 50 mL/minute) to give 55 mg of the title compound. ¹H NMR (400 MHz, DMSO-d₆) δ ppm 8.89 (d, J=2.3 Hz, 1H), 8.54 (dt, J=21.4, 5.9 Hz, 1H), 8.17 (dd, J=8.3, 2.7 Hz, 1H), 7.91 (d, J=6.0 Hz, 3H), 7.48 (d, J=8.3 Hz, 1H), 4.80 (d, J=5.4 Hz, 1H), 4.53 (d, J=5.8 Hz, 1H), 4.46 (d, J=5.9 Hz, 2H), 2.63 (dd, J=9.0, 4.5 Hz, 1H), 2.12-2.02 (m, 1H), 1.97 (ddd, J=28.9, 13.2, 7.8 Hz, 1H), 1.89-1.73 (m, 1H), 1.70 (dd, J=12.5, 9.1 Hz, 1H), 1.59-1.49 (m, 1H).

### Example 214B: (2R,4R)-6-chloro-4-hydroxy-N-[(1RS,2SR,4RS,5SR)-5-({[5-(trifluoromethyl)pyridin-2-yl]methyl}carbamoyl)-7-oxabicyclo[2.2.1]heptan-2-yl]-3,4-dihydro-2H-1-benzopyran-2-carboxamide

To a solution of the product of Example 214A (53 mg, 0.081 mmol), the product of Example 1B (21.23 mg, 0.094 mmol), and N-ethyl-N-isopropylpropan-2-amine (0.071 mL, 0.407 mmol) in N,N-dimethylformamide (1.5 mL), 2-(3H-[1,2,3]triazolo[4,5-b]pyridin-3-yl)-1,1,3,3-tetramethylisouronium hexafluorophosphate(V) (38.7 mg, 0.102 mmol) was added and the mixture was stirred at ambient temperature for 90 minutes. Volatiles were removed under high vacuum and the residue was dissolved in methanol (with a few drops of dichloromethane to completely dissolve) and treated with sodium tetrahydroborate (3.08 mg, 0.081 mmol) at ambient temperature for 30 minutes. Solvent was removed and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 35 mg of the title compound. ¹H NMR (500 MHz, DMSO-d₆) δ ppm 8.89 (dq, J=2.0, 1.0 Hz, 1H), 8.50 (td, J=6.0, 2.5 Hz, 1H), 8.18 (ddt, J=8.2, 2.2, 1.0 Hz, 1H), 7.96 (dd, J=13.2, 6.9 Hz, 1H), 7.53 7.45 (m, 1H), 7.42-7.36 (m, 1H), 7.19 (dtd, J=8.7, 2.8, 0.7 Hz, 1H), 6.88 (dd, J=8.7, 0.9 Hz, 1H), 4.84-4.76 (m, 1H), 4.71 (d, J=5.4 Hz, 1H), 4.65 (ddd, J=11.8, 4.5, 2.3 Hz, 1H), 4.45 (d, J=5.9 Hz, 2H), 4.34 (dd, J=8.1, 5.6 Hz, 1H), 3.88 (tt, J=8.1, 3.3 Hz, 1H), 2.60 (dd, J=9.0, 4.6 Hz, 1H), 2.36-2.28 (m, 1H), 2.03-1.95 (m, 2H), 1.81-1.70 (m, 1H), 1.66 (dddd, J=12.4, 10.1, 6.1, 3.8 Hz, 2H); MS (APCI⁺) m/z 525.98 (M+H)⁺.

### Example 215: (2R,4R)-6-chloro-4-hydroxy-N-(3-{3-[cis-3-(trifluoromethoxy)cyclobutyl]-1H-pyrazol-1-yl}bicyclo[1.1.1]pentan-1-yl)-3,4-dihydro-2H-1-benzopyran-2-carboxamide (Compound 314)

### Example 215A: 3-(dimethylamino)-1-(cis-3-(trifluoromethoxy)cyclobutyl)prop-2-en-1-one

The reaction and purification conditions described in Example 199A substituting the product of Example 193B for tert-butyl (3-acetylbicyclo[1.1.1]pentan-1-yl)carbamate gave the title compound, which was used as is in the next step without further characterization or purification.

### Example 215B: 3-(cis-3-(trifluoromethoxy)cyclobutyl)-1H-pyrazole

To a solution of the product of Example 215A (695 mg, 2.93 mmol) in anhydrous methanol (10 mL) was added hydrazine hydrate (64% aqueous solution, 0.213 mL). The reaction mixture was stirred at 60° C. for 18 hours, cooled to ambient temperature and then concentrated under reduced pressure. The resulting residue was purified by chromatography on silica gel (0-100% ethyl acetate in isohexane) to give the title compound. MS (ESI⁺) ml 207 (M+H)⁺.

### Example 215C: methyl 3-(bis(tert-butoxycarbonyl)amino)bicyclo[1.1.1]pentane-1-carboxylate

Triethylamine (4.57 mL, 32.8 mmol) was added to a suspension of methyl 3-aminobicyclo[1.1.1]pentane-1-carboxylate hydrochloride (2.45 g, 13.11 mmol, Fluorochem) in dichloromethane (68 mL). Di-tert-butyl dicarbonate (4.56 mL, 19.66 mmol) was then added, and the reaction mixture was stirred at room temperature for 3 days. Dichloromethane (68 mL) was added to the reaction, and the mixture was washed with water (2×100 mL). The organic phase was dried over magnesium sulfate and concentrated in vacuo. The residue was taken up in acetonitrile (20 mL). 4-Dimethylaminopyridine (0.32 g, 2.62 mmol) and di-tert-butyl dicarbonate (4.56 mL, 19.66 mmol) were added. The reaction mixture was stirred at ambient temperature overnight. Water (100 mL) was added, and the resulting suspension was extracted with ethyl acetate (3×100 mL). The combined organic layers were washed with brine, dried over magnesium sulfate, filtered and concentrated to give the title compound (4.75 g, 12.52 mmol, 96% yield). ¹H NMR (500 MHz, DMSO-d₆) δ ppm 3.61 (s, 3H), 2.33 (s, 6H), 1.44 (s, 18H).

### Example 215D: 3-(bis(tert-butoxycarbonyl)amino)bicyclo[1.1.1]pentane-1-carboxylic Acid

The reaction and purification conditions described in Example 117B substituting the product of Example 215C for the product of Example 117A gave the title compound. ¹H NMR (500 MHz, DMSO-d₆) δ ppm 12.55 (br s, 1H), 2.29 (s, 6H), 1.44 (s, 18H).

### Example 215E: mesityl-λ³-iodanediyl bis(3-(bis(tert-butoxycarbonyl)amino)bicyclo[1.1.1]pentane-1-carboxylate

A solution of the product of Example 215D (1.08 g, 3.30 mmol) and iodomesitylene diacetate (0.60 g, 1.65 mmol) in toluene (10 mL) was stirred at 60° C. for 30 minutes. The solvent was removed under reduced pressure and azeotroped with toluene (4×5 mL) to give the title compound (1.56 g, 1.65 mmol, 100% yield). ¹H NMR (500 MHz, DMSO-d₆) δ ppm 7.09 (s, 2H), 2.70 (s, 6H), 2.39 (s, 3H), 2.32 (s, 12H), 1.49 (s, 36H).

### Example 215F: di-tert-butyl (3-{3-[cis-3-(trifluoromethoxy)cyclobutyl]-1H-pyrazol-1-yl}bicyclo[1.1.1]pentan-1-yl)-2-imidodicarbonate

Dioxane (3 mL) was added to a mixture of the product of Example 215E (290 mg, 0.323 mmol) and the product of Example 215B (93 mg, 0.452 mmol). The resulting mixture was degassed under vacuum and then sonicated until all solids were dissolved. Copper(I) thiophene-2-carboxylate (61.6 mg, 0.323 mmol) was added in one portion. The mixture was sonicated for 2 minutes, and then stirred at ambient temperature for 15 minutes. To the reaction mixture was added saturated aqueous sodium bicarbonate (50 mL) and ethyl acetate (50 mL). The layers were separated and the organic layer was washed with additional saturated aqueous sodium bicarbonate (10 mL) and brine (10 mL). The organic phase was dried over magnesium sulfate, filtered, and concentrated in vacuo. The resulting residue was purified by flash chromatography on silica gel (0-40% ethyl acetate in hexanes) to give the title compound (16 mg, 0.032 mmol, 10% yield). ¹H NMR (500 MHz, DMSO-d₆) δ ppm 7.35 (d, J=2.3 Hz, 1H), 6.21 (d, J=2.3 Hz, 1H), 4.64 (p, J=7.5 Hz, 1H),3.21-3.11 (m, 1H), 2.85-2.76 (m, 2H), 2.68 (s, 6H), 2.44-2.36 (m, 2H), 1.54 (s, 18H); MS (ESI⁺) m/z 488 (M+H)⁺.

### Example 215G: (1R,4R)-6-chloro-4-hydroxy-N-(3-{3-[cis-3-(trifluoromethoxy)cyclobutyl]-1H-pyrazol-1-yl}bicyclo[1.1.1]pentan-1-yl)-3,4-dihydro-2H-1-benzopyran-2-carboxamide

The reaction and purification conditions described in Example 3C substituting the product of Example 215F for the product of Example 3A gave the title compound. ¹H NMR (500 MHz, DMSO-d₆) δ ppm 8.88 (s, 1H), 7.68 (d, J=2.3 Hz, 1H), 7.39 (d, J=2.7 Hz, 1H), 7.21 (dd, J=8.7, 2.7 Hz, 1H), 6.90 (d, J=8.7 Hz, 1H), 6.25 (d, J=2.3 Hz, 1H), 5.72 (d, J=6.3 Hz, 1H), 4.87-4.75 (m, 2H), 4.65 (dd, J=12.0, 2.3 Hz, 1H), 3.12-3.02 (m, 1H), 2.74-2.66 (m, 2H), 2.48 (s, 6H), 2.42-2.34 (m, 1H), 2.32-2.23 (m, 2H), 1.78-1.67 (m, 1H); MS (ESI⁺) m/z 498 (M+H)⁺.

### Example 216: (2R,4R)-6-chloro-4-hydroxy-N-(3-{1-[cis-3-(trifluoromethoxy)cyclobutyl]-1H-1,2,3-triazol-4-yl}bicyclo[1.1.1]pentan-1-yl)-3,4-dihydro-2H-1-benzopyran-2-carboxamide (Compound 315)

### Example 216A: Tert-Butyl (3-(2H-1,2,3-triazol-4-yl)bicyclo[1.1.1]pentan-1-yl)carbamate

To a mixture of the product of Example 151A (184 mg, 0.728 mmol) and copper(II) sulfate (1.3 mg, 0.008 mmol) in tert-butanol (7.8 mL) and water (2.6 mL) in a sealed tube was added azidotrimethylsilane (0.103 mL, 0.78 mmol), benzoic acid (8.9 mg, 0.073 mmol) and sodium ascorbate (2.6 mg, 0.013 mmol) at ambient temperature. The tube was flushed with nitrogen, sealed, and stirred at 80° C. for 3 days. The mixture was cooled to ambient temperature, poured onto ice water (25 mL) and extracted with ethyl acetate (3×25 mL). The combined organic fractions were washed with brine (25 mL), dried over magnesium sulfate, filtered, and concentrated in vacuo to give the title compound that was used without further purification or characterization (498 mg, estimated 73% purity based on mass recovery).

### Example 216B: Tert-Butyl (3-(1-(cis-3-(trifluoromethoxy)cyclobutyl)-1H-1,2,3-triazol-4-yl)bicyclo[1.1.1]pentan-1-yl)carbamate and Tert-Butyl (3-(1-(cis-3-(trifluoromethoxy)cyclobutyl)-1H-1,2,3-triazol-5-yl)bicyclo[1.1.1]pentan-1-yl)carbamate 1:1

To a mixture of the product of Example 217D (68.3 mg, 0.437 mmol), the product of Example 216A (150 mg, 0.437 mmol) and triphenylphosphine (229 mg, 0.875 mmol) in tetrahydrofuran (3.5 mL) at 0° C. was added diisopropyl azodicarboxylate (0.172 mL, 0.875 mmol) in a dropwise fashion. The reaction mixture was stirred at ambient temperature for 20 hours and then concentrated in vacuo. The residue was purified by chromatography on silica gel (0-100% ethyl acetate in cyclohexane) to give the title compounds (49 mg, 0.09 mmol, 20% yield). MS (ESI) m/z 390 (M+H)⁺.

### Example 216C: 3-(1-(cis-3-(trifluoromethoxy)cyclobutyl)-1H-1,2,3-triazol-4-yl)bicyclo[1.1.1]pentan-1-amine and 3-(1-(cis-3-(trifluoromethoxy)cyclobutyl)-1H-1,2,3-triazol-5-yl)bicyclo[1.1.1]pentan-1-amine (1:1)

To a solution of the products of Example 216B (49 mg, 0.088 mmol) in dichloromethane (1 mL) at ambient temperature was added trifluoroacetic acid (0.13 mL) and the reaction mixture was stirred at ambient temperature for 20 hours. The resulting mixture was concentrated in vacuo, taken up in methanol (2 mL), combined with SCX resin (0.2 g), and then loaded onto a column packed with 0.3 g SCX resin. The column was first washed with methanol (10 mL). The resin column was then eluted with ammonia in methanol (0.7 M, 10 mL) and the filtrate was concentrated in vacuo to afford the title compound (23 mg, 0.08 mmol, 90% yield). MS (ESI) m/z 290 (M+H)⁺.

### Example 216D: (2R)-6-chloro-4-oxo-N-(3-{1-[cis-3-(trifluoromethoxy)cyclobutyl]-1H-1,2,3-triazol-4-yl}bicyclo[1.1.1]pentan-1-yl)-3,4-dihydro-2H-1-benzopyran-2-carboxamide

The title compound was synthesized using the same procedure as described in Example 155C, substituting the product of Example 216C for the product of Example 155B, and the product of Example 1B for the product of Example 3B, and purified by the following preparative HPLC method: [Waters XSelect® C18 5 μm CSH column, 30×100 mm, 40-70% gradient of acetonitrile in buffer (0.1% formic acid)]. MS (ESI) m/z 497 (M+H)⁺.

### Example 216E: (2R,4R)-6-chloro-4-hydroxy-N-(3-{1-[cis-3-(trifluoromethoxy)cyclobutyl]-1H-1,2,3-triazol-4-yl}bicyclo[1.1.1]pentan-1-yl)-3,4-dihydro-2H-1-benzopyran-2-carboxamide

The reaction and purification conditions described in Example 62 substituting the product of Example 216D for the product of Example 53 gave the title compound. ¹H NMR (500 MHz, methanol-d₄) δ ppm 8.00 (s, 1H), 7.47-7.41, (m, 1H), 7.17 (dd, J=8.8, 2.7 Hz, 1H), 6.94 (d, J=8.7 Hz, 1H), 4.98-4.91 (m, 1H), 4.87-4.73 (m, 2H), 4.63 (dd, J=11.7, 2.4 Hz, 1H), 3.16-3.05 (m, 2H), 2.93-2.80 (m, 2H), 2.60-2.54 (m, 1H), 2.48 (s, 6H), 1.94-1.85 (m, 1H); MS (ESI⁺) m/z 499 (M+H)⁺.

### Example 217: (2R,4R)-6-chloro-4-hydroxy-N-(3-{1-[cis-3-(trifluoromethoxy)cyclobutyl]-1H-pyrazol-4-yl}bicyclo[1.1.1]pentan-1-yl)-3,4-dihydro-2H-1-benzopyran-2-carboxamide (Compound 316)

### Example 217A: trans-3-(benzyloxy)cyclobutyl 4-nitrobenzoate

To a solution of the product of Example 201A (10.0 g, 50.5 mmol), 4-nitrobenzoic acid (8.44 g, 50.5 mmol), and triphenylphosphine (13.2 g, 50.5 mmol) in toluene (200 mL) was added diisopropyl azodicarboxylate (9.82 mL, 50.5 mmol) dropwise at 0° C. The mixture was stirred at 20° C. for 16 hours. Then the reaction mixture was combined with another batch of the same reaction mixture, diluted with water (300 mL), and extracted with ethyl acetate (3×300 mL). The combined organic layers were washed with brine (200 mL), dried over Na₂SO₄, and concentrated under reduced pressure. The residue was purified by column chromatography on silica gel (petroleum ether:ethyl acetate=20:1 to 8:1) to afford the title intermediate (27.0 g, 74.2 mmol, 74% yield). ¹H NMR (400 MHz, DMSO-d₆) δ ppm 8.35 (d, J=8.77 Hz, 2H), 8.20 (d, J=8.77 Hz, 2H), 7.26-7.37 (m, 5H), 5.28-5.36 (m, 1H), 4.42 (s, 2H), 4.34 (quin, J=5.92 Hz, 1H), 2.45-2.49 (m, 4H).

### Example 217B: trans-3-(benzyloxy)cyclobutanol

To a solution of the product of Example 217A (15 g, 41 mmol) in tetrahydrofuran (150 mL) was added a solution of NaOH (2.0 g, 50 mmol) in water (38 mL) dropwise at 0° C. The reaction mixture was stirred at 20° C. for 10 hours. The reaction mixture was combined with another batch of the same reaction mixture and was concentrated in vacuo. The residue was extracted with ethyl acetate (3×150 mL). The combined organic layers were washed with brine (150 mL) and concentrated to afford the title intermediate (15 g, 72 mmol, 96% yield). ¹H NMR (400 MHz, DMSO-d₆) δ ppm 7.24-7.38 (m, 5H), 4.98 (d, J=4.82 Hz, 1H), 4.33 (s, 2H), 4.24-4.32 (m, 1H), 4.11-4.18 (m, 1H), 2.13-2.23 (m, 2H), 1.97-2.07 (m, 2H).

### Example 217C: ((trans-3-(trifluoromethoxy)cyclobutoxy)methyl)benzene

The title compound was synthesized using the same procedure as described in Example 13O substituting the product of Example 13N with the product of Example 217B and increasing the reaction time to 48 hours. ¹H NMR (400 MHz, DMSO-d₆) δ ppm 7.23-7.40 (m, 5H), 4.89-5.00 (m, 1H), 4.38 (s, 2H), 4.19-4.29 (m, 1H), 2.43 (t,=5.69 Hz, 4H), 2.39-2.40 (m, 1H).

### Example 217D: trans-3-(trifluoromethoxy)cyclobutanol

To a solution of the product of Example 217D (12.0 g, 41.4 mmol) in tetrahydrofuran (120 mL) was added 10% palladium on carbon (8.82 g, 4.14 mmol, 50% water) under argon, and the reaction mixture was stirred at 50° C. under hydrogen (50 psi) for 48 hours. Then the suspension was filtered through a pad of diatomaceous earth and the pad was washed with ethyl acetate (50 mL×3). The filtrate was concentrated under reduced pressure to afford the title intermediate (5.80 g, 30.7 mmol, 74% yield). ¹H NMR (400 MHz, DMSO-d₆) δ ppm 5.24 (d, J=5.14 Hz, 1H), 4.86-4.99 (m, 1H), 4.28-4.41 (m, 1H), 2.33-2.46 (m, 2H), 2.18-2.29 (m, 2H).

### Example 217E: trans-3-(trifluoromethoxy)cyclobutyl methanesulfonate

To a solution of the product of Example 217D (0.055 g, 0.36 mmol) and Hunig's base (N,N-diisopropylethylamine) (0.093 mL, 0.53 mmol) in dichloromethane (1.5 mL) at 0° C. under nitrogen was added methanesulfonyl chloride (0.033 mL, 0.43 mmol) dropwise. The reaction mixture stirred at this temperature for 30 minutes and then at ambient temperature for 30 minutes. The reaction mixture was quenched with saturated NH₄Cl (aqueous) (2.5 mL) and the phases were separated. The aqueous phase was extracted with additional dichloromethane (2.5 mL). The combined organic layers were concentrated in vacuo to afford the crude title intermediate (0.11 g, 0.35 mmol, quantitative yield), which was carried forward without purification. ¹H NMR (500 MHz, DMSO-d₆) δ ppm 5.22 (p, J=6.0 Hz, 1H), 5.04 (p, J=5.7 Hz, 1H), 3.20 (s, 3H), 2.73-2.68 (m, 4H).

### Example 217F: 3-benzyl-3H-1,2,3-oxadiazol-1-ium-5-olate

Behind a blast shield, to a solution of 2-(benzylamino)acetic acid (250 mg, 1.51 mmol) in 1,2-dimethoxyethane (7.0 mL) under nitrogen was added isoamyl nitrite (0.204 mL, 1.51 mmol). The reaction mixture stirred for 2 hours and was concentrated in vacuo (water bath at 30° C. to prevent decomposition). The crude residue was dispersed in dichloromethane:isohexane (1:15), concentrated in vacuo, and triturated using isohexane to afford 2-(benzyl(nitroso)amino)acetic acid.

Behind a blast shield, to a solution of 2-(benzyl(nitroso)amino)acetic acid (294 mg, 1.51 mmol) in dichloromethane (7.00 mL) at 0° C. under nitrogen was added trifluoroacetic anhydride (0.214 mL, 1.51 mmol) dropwise. The reaction mixture was warmed to ambient temperature and stirred for 1.5 hours. Then water (7 mL) was added and the excess trifluoroacetic anhydride was quenched with sodium hydrogen carbonate. The phases were separated, and the aqueous layer was further extracted with dichloromethane (10 mL). The combined organic layers were dried over Na₂SO₄, filtered, and concentrated in vacuo to afford a crude amorphous solid, which was dispersed in dichloromethane:isohexane (1:15). This solution was then concentrated in vacuo to afford the title intermediate (202 mg, 1.03 mmol, 68% yield). ¹H NMR (500 MHz, CDCl₃) δ ppm 7.47 (dd, J=5.0, 2.0 Hz, 3H), 7.41-7.35 (m, 2H), 6.17 (s, 1H), 5.35 (s, 2H).

### Example 217G: Tert-Butyl (3-(1H-benzyl-1H-pyrazol-4-yl)bicyclo[1.1.1]pentan-1-yl)carbamate

To a suspension of the product of Example 217F (250 mg, 1.42 mmol), the product of Example 212C (588 mg, 2.84 mmol), sodium 4,4′-(1,10-phenanthroline-4,7-diyl)dibenzenesulfinate (752 mg, 1.49 mmol), sodium L-ascorbate (562 mg, 2.84 mmol), and triethylamine (0.791 mL, 5.68 mmol) in water (5.0 mL) and t-butanol (7.5 mL) was added copper(II) sulfate (238 mg, 1.49 mmol) as a solution in water (2.5 mL). The reaction mixture was heated to 85° C. and stirred for 20 hours. Then the reaction mixture was cooled to ambient temperature and brine (20 mL) was added, followed by ethyl acetate (20 mL). The phases were separated, and the aqueous layer was further extracted with ethyl acetate (20 mL). The combined organic layers were dried over Na₂SO₄, filtered, and concentrated in vacuo. The residue was purified by chromatography on silica gel (0-100% ethyl acetate in isohexane) to afford the title intermediate (395 mg, 1.09 mmol, 77% yield). ¹H NMR (500 MHz, DMSO-d₆) δ ppm 7.60 (s, 1H), 7.36-7.19 (m, 6H), 5.23 (s, 2H), 2.06 (s, 6H), 1.37 (s, 9H); MS (ESI⁺) m/z 340 (M+H)⁺.

### Example 217H: Tert-Butyl (3-(1H-pyrazol-4-yl)bicyclo[1.1.1]pentan-1-yl)carbamate

A solution of the product of Example 217G (200 mg, 0.589 mmol) in acetic acid (6 mL) was flowed through an H-Cube® continuous flow hydrogenator (1 mL/minute) with a 10% palladium on carbon catalyst cartridge at 100° C. using controlled H₂ mode (100 bar) as a continuous loop for 20 hours. The reaction mixture was then cooled to ambient temperature and diluted with water (20 mL) and ethyl acetate (20 mL). The phases were separated, and the aqueous phase was extracted with additional ethyl acetate (20 mL). The combined organic layers were washed with brine (3×20 mL), dried over Na₂SO₄, filtered, concentrated in vacuo, and purified by chromatography on silica gel (0-100% ethyl acetate in cyclohexane) to afford the title intermediate (37 mg, 0.14 mmol, 24% yield). MS (ESI⁺) m/z 250 (M+H)⁺.

### Example 217I: Tert-Butyl (3-(1-(cis-3-(trifluoromethoxy)cyclobutyl)-1H-pyrazol-4-yl)bicyclo[1.1.1]pentan-1-yl)carbamate

A solution of the product of Example 217H (37 mg, 0.15 mmol), cesium carbonate (145 mg, 0.445 mmol), and the product of Example 217E (87 mg, 0.37 mmol) in N,N-dimethyl formamide (0.5 mL), under nitrogen, was heated to 80° C. and stirred for 22 hours. Then the reaction mixture was diluted with water (10 mL) and ethyl acetate (10 ml), and the phases were separated. The organic phase was washed with 1:1 brine:H₂O (3×15 mL), dried over Na₂SO₄, filtered, and then concentrated in vacuo to afford a crude residue (73 mg). The crude residues from 2 batches of the same reaction were combined and purified by chromatography on silica gel (0-100% ethyl acetate in cyclohexane) to afford the title intermediate (11 mg, 0.027 mmol, 12% yield). ¹H NMR (500 MHz, CDCl₃) δ ppm 7.39 (s, 1H), 7.26 (s, 1H), 4.59-4.50 (m, 1H), 4.44-4.34 (m, 1H), 3.03-2.95 (m, 2H), 2.90-2.82 (m, 2H), 2.24 (s, 6H), 1.48 (s, 9H).

### Example 217J: 3-(1-(cis-3-(trifluoromethoxy)cyclobutyl)-1H-pyrazol-4-yl)bicyclo[1.1.1]pentan-1-amine

To a solution of the product of Example 217I (11 mg, 0.028 mmol) in dichloromethane (1.0 mL) was added trifluoroacetic acid (0.098 mL, 1.3 mmol) and the reaction mixture was stirred for 3 hours. The solvent was removed under vacuum and co-evaporated with toluene (3×5 mL) to afford a crude salt, which was purified on SCX resin (washing with methanol then eluted with 0.7M ammonia in methanol) to afford the title intermediate (8.0 mg, 0.026 mmol, 93% yield). ¹H NMR (500 MHz, CDCl₃) δ ppm 7.36 (s, 1H), 7.24 (s, 1H), 4.58-4.48 (m, 1H), 4.42-4.31 (m, 1H), 3.01-2.92 (m, 2H), 2.89-2.79 (m, 2H), 2.05 (s, 6H); MS (ESI⁺) m/z 289 (M+H)⁺.

### Example 217K: (2R)-6-chloro-4-oxo-N-(3-{1-[cis-3-(trifluoromethoxy)cyclobutyl]-1H-pyrazol-4-yl}bicyclo[1.1.1]pentan-1-yl)-3,4-dihydro-2H-1-benzopyran-2-carboxamide

To a solution of the product of Example 217J (8.0 mg, 0.028 mmol), the product of Example 1B (9.5 mg, 0.042 mmol), and triethylamine (0.023 mL, 0.17 mmol) in N,N-dimethylformamide (0.5 mL) was added HATU (1-[bis(dimethylamino)methylene]-1H-1,2,3-triazolo[4,5-b]pyridinium 3-oxid hexafluorophosphate) (16 mg, 0.042 mmol). After the reaction mixture was stirred for 1 hour, it was quenched with saturated aqueous sodium bicarbonate solution (2.5 mL) and the aqueous phase was extracted with dichloromethane (2×2 mL). The combined organic phases were then concentrated in vacuo to afford the crude title intermediate (14 mg, 0.028 mmol, quantitative yield), which was carried forward without further purification. MS (ESI⁺) m/z 496 (M+H)⁺.

### Example 217L: (24R)-6-chloro-4-hydroxy-N-(3-{-[cis-3-(trifluoromethoxy)cyclobutyl]-1H-pyrazol-4-yl}bicyclo[1.1.1]pentan-1-yl)-3,4-dihydro-2H-1-benzopyran-2-carboxamide

To a solution of Example 217K (14 mg, 0.028 mmol) in methanol (0.5 mL) at ambient temperature under nitrogen, was added sodium borohydride (13 mg, 0.34 mmol), and the reaction mixture was stirred for 15 minutes. Then the reaction mixture was quenched with saturated NH₄Cl (aqueous) (2.5 mL), stirred for 10 minutes, and then was extracted with dichloromethane (2×2 mL). The combined organic phases were concentrated in vacuo and purified by chromatography on silica gel (50-100% ethyl acetate in isohexane) to afford the title compound (8.4 mg, 0.016 mmol, 57% yield). ¹H NMR (500 MHz, DMSO-d₆) δ ppm 8.66 (s, 1H), 7.72 (d, J=0.8 Hz, 1H), 7.39 (dd, J=2.8, 1.0 Hz, 1H), 7.38 (s, 1H), 7.21 (dd, J=8.7, 2.7 Hz, 1H), 6.89 (d, J=8.7 Hz, 1H), 5.70 (d, J=6.3 Hz, 1H), 4.85-4.79 (m, 1H), 4.77-4.70 (m, 1H), 4.62-4.57 (m, 1H), 4.54-4.46 (m, 1H), 2.93-2.85 (m, 2H), 2.72-2.63 (m, 2H), 2.39-2.33 (m, 1H), 2.22 (s, 6H), 1.75-1.66 (m, 1H); ¹⁹F NMR (471 MHz, DMSO-d₆) δ ppm −57.95; MS (ESI⁺) m/z 498 (M+H)⁺.

TABLE 3
The following compounds can be prepared using methodologies similar to those described in the above examples.

| | |
|---|---|
| 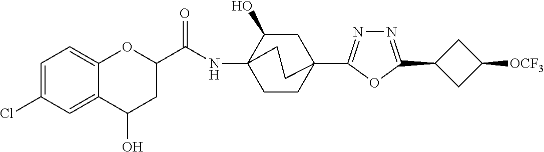 [SMILES: O=C(NC12CCC(c3nnc([C@H]4C[C@@H](OC(F)(F)F)C4)o3)(CC1)C[C@@H]2O)C1CC(O)c2cc(Cl)ccc2O1] | 6-chloro-4-hydroxy-N-[(2S)-2-hydroxy-4-{5- [(1s,3R)-3-(trifluoromethoxy)cyclobutyl]- 1,3,4-oxadiazol-2-yl}bicyclo[2.2.2]octan- 1-yl]-3,4-dihydro-2H-1-benzopyran- 2-carboxamide |
| 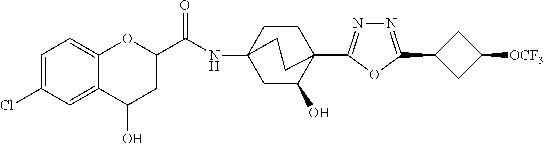 [SMILES: O=C(NC12CCC(c3nnc([C@H]4C[C@@H](OC(F)(F)F)C4)o3)(CC1)[C@@H](O)C2)C1CC(O)c2cc(Cl)ccc2O1] | 6-chloro-4-hydroxy-N-[(3S)-3-hydroxy- 4-{5-[(1s,3R)-3-(trifluoromethoxy)cyclobutyl]- 1,3,4-oxadiazol-2-yl}bicyclo[2.2.2]octan- 1-yl]-3,4-dihydro-2H-1- benzopyran-2-carboxamide |
| 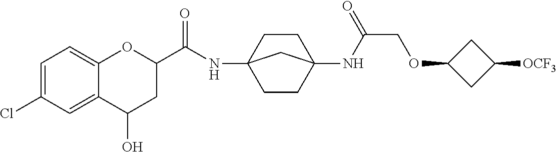 [SMILES: O=C(CO[C@H]1C[C@@H](OC(F)(F)F)C1)NC12CCC(NC(=O)C3CC(O)c4cc(Cl)ccc4O3)(CC1)C2] | 6-chloro-4-hydroxy-N-[4-(2-{[(1s,3s)- 3-(trifluoromethoxy)cyclobutyl]oxy} acetamido)bicyclo[2.2.1]heptan-l-yl]-3,4- dihydro-2H-1-benzopyran-2- carboxamide |
| 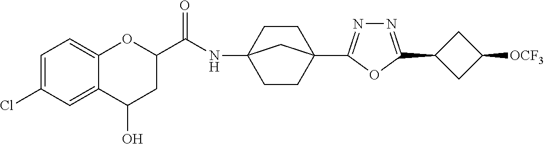 [SMILES: O=C(NC12CCC(c3nnc([C@H]4C[C@@H](OC(F)(F)F)C4)o3)(CC1)C2)C1CC(O)c2cc(Cl)ccc2O1] | 6-chloro-4-hydroxy-N-(4-{5-[(1s,3s)-3- (trifluoromethoxy)cyclobutyl]-1,3,4- oxadiazol-2-yl}bicyclo[2.2.1]heptan-1-yl)- 3,4-dihydro-2H-1-benzopyran-2- carboxamide |
| 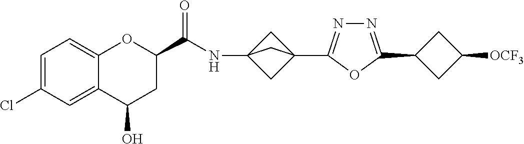 [SMILES: O=C(NC12CC(c3nnc([C@H]4C[C@@H](OC(F)(F)F)C4)o3)(C1)C2)[C@H]1C[C@@H](O)c2cc(Cl)ccc2O1] | (2R,4R)-6-chloro-4-hydroxy-N-(3-{5- [(1s,3S)-3-(trifluoromethoxy)cyclobutyl]- 1,3,4-oxadiazol-2-yl}bicyclo[1.1.1]pentan- 1-yl)-3,4-dihydro-2H-1-benzopyran- 2-carboxamide |
| 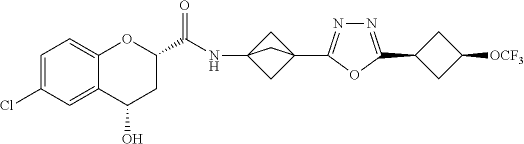 [SMILES: O=C(NC12CC(c3nnc([C@H]4C[C@@H](OC(F)(F)F)C4)o3)(C1)C2)[C@@H]1C[C@H](O)c2cc(Cl)ccc2O1] | (2S,4S)-6-chloro-4-hydroxy-N-(3-{5- [(1s,3R)-3-(trifluoromethoxy)cyclobutyl]-1, 3,4-oxadiazol-2-yl}bicyclo[1.1.1]pentan-1-yl)- 3,4-dihydro-2H-1-benzopyran-2- carboxamide |
| 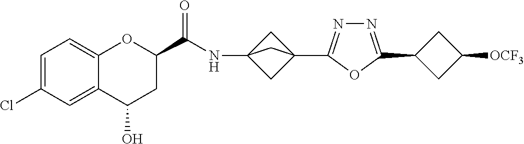 [SMILES: O=C(NC12CC(c3nnc([C@H]4C[C@@H](OC(F)(F)F)C4)o3)(C1)C2)[C@H]1C[C@H](O)c2cc(Cl)ccc2O1] | (2R,4S)-6-chloro-4-hydroxy-N-(3-{5-[(1s, 3S)-3-(trifluoromethoxy)cyclobutyl]-1,3,4- oxadiazol-2-yl}bicyclo[1.1.1]pentan-1-yl)-3,4- dihydro-2H-1-benzopyran-2-carboxamide |
| 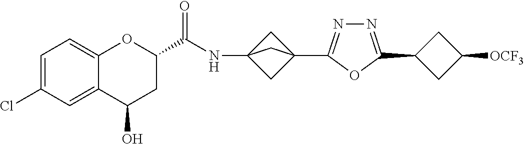 [SMILES: O=C(NC12CC(c3nnc([C@H]4C[C@@H](OC(F)(F)F)C4)o3)(C1)C2)[C@@H]1C[C@@H](O)c2cc(Cl)ccc2O1] | (2S,4R)-6-chloro-4-hydroxy-N-(3-{5-[(1s, 3R)-3-(trifluoromethoxy)cyclobutyl]-1,3,4- oxadiazol-2-yl}bicyclo[1.1.1]pentan-1-yl)-3,4- dihydro-2H-1-benzopyran-2-carboxamide |
| 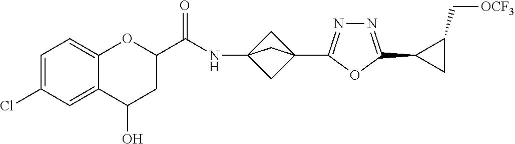 [SMILES: O=C(NC12CC(c3nnc([C@@H]4C[C@H]4COC(F)(F)F)o3)(C1)C2)C1CC(O)c2cc(Cl)ccc2O1] | 6-chloro-4-hydroxy-N-[3-(5-{(1R,2R)-2- [(trifluoromethoxy)methyl]cyclopropyl}-1,3,4- oxadiazol-2-yl)bicyclo[1.1.1]pentan-1-yl]-3,4- dihydro-2H-1-benzopyran-2-carboxamide |
| 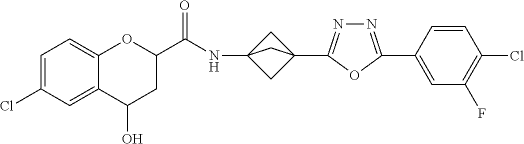 [SMILES: O=C(NC12CC(c3nnc(-c4ccc(Cl)c(F)c4)o3)(C1)C2)C1CC(O)c2cc(Cl)ccc2O1] | 6-chloro-N-{3-[5-(4-chloro-3-fluorophenyl)- 1,3,4-oxadiazol-2-yl]bicyclo[1.1.1]pentan- 1-yl}-4-hydroxy-3,4-dihydro-2H-1-benzopyran- 2-carboxamide |
| 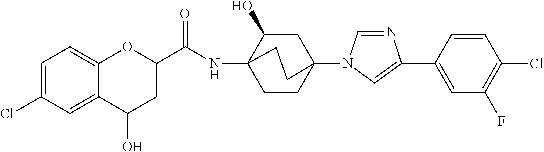 [SMILES: O=C(NC12CCC(n3cnc(-c4ccc(Cl)c(F)c4)c3)(CC1)C[C@@H]2O)C1CC(O)c2cc(Cl)ccc2O1] | 6-chloro-N-{(2S)-4-[4-(4-chloro-3- fluorophenyl)-1H-imidazol-1-yl]-2- hydroxybicyclo[2.2.2]octan-1-yl}-4- hydroxy-3,4-dihydro-2H-1-benzopyran-2- carboxamide |
| 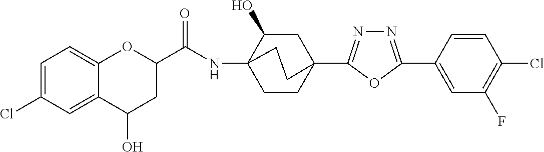 [SMILES: O=C(NC12CCC(c3nnc(-c4ccc(Cl)c(F)c4)o3)(CC1)C[C@@H]2O)C1CC(O)c2cc(Cl)ccc2O1] | 6-chloro-N-{(2S)-4-[5-(4-chloro-3- fluorophenyl)-1,3,4-oxadiazol-2-yl]-2- hydroxybicyclo[2.2.2]octan-1- yl}-4-hydroxy-3,4-dihydro-2H-1- benzopyran-2-carboxamide |
| 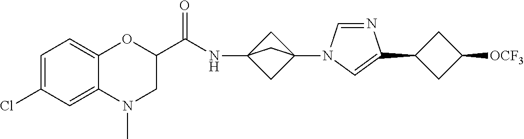 [SMILES: CN1CC(C(=O)NC23CC(n4cnc([C@H]5C[C@@H](OC(F)(F)F)C5)c4)(C2)C3)Oc2ccc(Cl)cc21] | 6-chloro-4-methyl-N-(3-{4-[(1s, 3s)-3-(trifluoromethoxy)cyclobutyl]-1H- imidazol-1-yl}bicyclo[1.1.1]pentan-1-yl)- 3,4-dihydro-2H-1,4-benzoxazine-2- carboxamide |
| 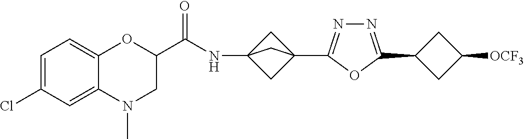 [SMILES: CN1CC(C(=O)NC23CC(c4nnc([C@H]5C[C@@H](OC(F)(F)F)C5)o4)(C2)C3)Oc2ccc(Cl)cc21] | 6-chloro-4-methyl-N-(3-{5-[(1s,3s)- 3-(trifluoromethoxy)cyclobutyl]-1,3,4- oxadiazol-2-yl}bicyclo[1.1.1]pentan-1-yl)- 3,4-dihydro-2H-1,4-benzoxazine-2- carboxamide |
| 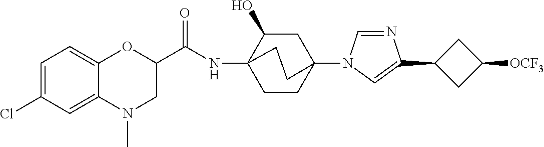 [SMILES: CN1CC(C(=O)NC23CCC(n4cnc([C@H]5C[C@@H](OC(F)(F)F)C5)c4)(CC2)C[C@@H]3O)Oc2ccc(Cl)cc21] | 6-chloro-N-[(2S)-2-hydroxy-4-{4- [(1s,3R)-3-(trifluoromethoxy)cyclobutyl]- 1H-imidazol-1-yl}bicyclo[2.2,2]octan-l-yl]- 4-methyl-3,4-dihydro-2H-1,4- benzoxazine-2-carboxamide |
| 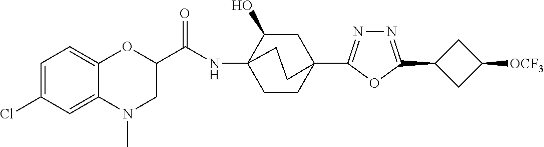 [SMILES: CN1CC(C(=O)NC23CCC(c4nnc([C@H]5C[C@@H](OC(F)(F)F)C5)o4)(CC2)C[C@@H]3O)Oc2ccc(Cl)cc21] | 6-chloro-N[(2S)-2-hydroxy-4-{5- [(1s,3R)-3-(trifluoromethoxy)cyclobutyl]- 1,3,4-oxadiazol-2-yl}bicyclo[2.2.2]octan-1- yl]4-methyl-3,4-dihydro-2H-1,4- benzoxazine-2-carboxamide |
| 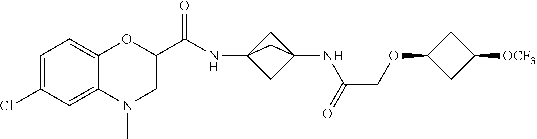 [SMILES: CN1CC(C(=O)NC23CC(NC(=O)CO[C@H]4C[C@@H](OC(F)(F)F)C4)(C2)C3)Oc2ccc(Cl)cc21] | 6-chloro-4-methyl-N-[3-(2-{[(1s,3s)-3- (trifluoromethoxy)cyclobutyl] oxy}acetamido)bicyclo [1.1.1]pentan-1-yl]-3,4-dihydro-2H- 1,4-benzoxazine-2- carboxamide |
| 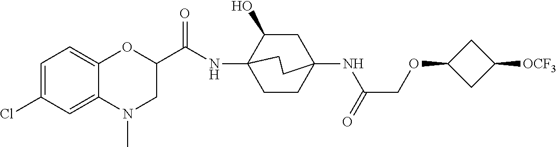 [SMILES: CN1CC(C(=O)NC23CCC(NC(=O)CO[C@H]4C[C@@H](OC(F)(F)F)C4)(CC2)C[C@@H]3O)Oc2ccc(Cl)cc21] | 6-chloro-N-[(2S)-2-hydroxy-4-(2-{[ (1s,3R)-3-(trifluoromethoxy)cyclobutyl]oxy} acetamido)bicyclo[2.2.2]octan-1-yl]-4- methyl-3,4-dihydro-2H-1,4- benzoxazine-2-carboxamide |
| 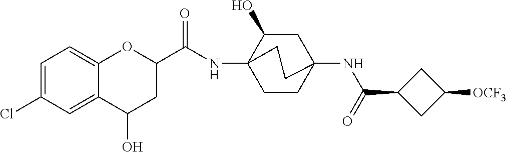 [SMILES: O=C(NC12CCC(NC(=O)[C@H]3C[C@@H](OC(F)(F)F)C3)(CC1)C[C@@H]2O)C1CC(O)c2cc(Cl)ccc2O1] | 6-chloro-4-hydroxy-N-[(2S)-2- hydroxy-4-{[(1s,3R)-3- (trifluoromethoxy)cyclobutane-1- carbonyl]amino}bicyclo[2.2.2] octan-1-yl]-3,4-dihydro- 2H-1-benzopyran-2-carboxamide |
| 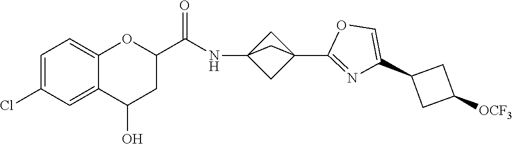 [SMILES: O=C(NC12CC(c3nc([C@H]4C[C@@H](OC(F)(F)F)C4)co3)(C1)C2)C1CC(O)c2cc(Cl)ccc2O1] | 6-chloro-4-hydroxy-N-(3-{4-[(1s,3s)- 3-(trifluoromethoxy)cyclobutyl]-1,3- oxazol-2-yl}bicyclo[1.1.1]pentan-1-yl)-3,4- dihydro-2H-1-benzopyran-2-carboxamide |
| 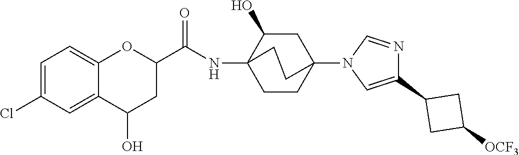 [SMILES: O=C(NC12CCC(n3cnc([C@H]4C[C@@H](OC(F)(F)F)C4)c3)(CC1)C[C@@H]2O)C1CC(O)c2cc(Cl)ccc2O1] | 6-chloro-4-hydroxy-N-[(2S)-2- hydroxy-4-{4-[(1s,3R)-3- (trifluoromethoxy)cyclobutyl]-1H-imidazol-1- yl}bicyclo[2.2.2]octan-1-yl]-3,4-dihydro- 2H-1-benzopyran-2-carboxamide |
| 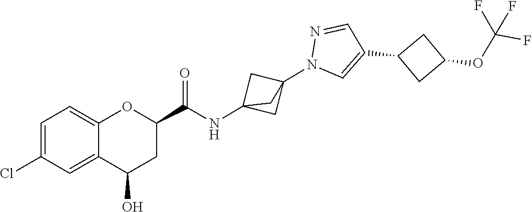 [SMILES: O=C(NC12CC(n3cc([C@H]4C[C@@H](OC(F)(F)F)C4)cn3)(C1)C2)[C@H]1C[C@@H](O)c2cc(Cl)ccc2O1] | (2R,4R)-6-chloro-4-hydroxy-N-(3-{4-[cis-3- (trifluoromethoxy)cyclobutyl]-1H-pyrazol-1- yl}bicyclo[1.1.1]pentan-1-yl)-3,4-dihydro- 2H-1-benzopyran-2-carboxamide |
| 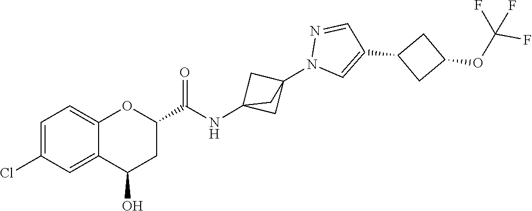 [SMILES: O=C(NC12CC(n3cc([C@H]4C[C@@H](OC(F)(F)F)C4)cn3)(C1)C2)[C@@H]1C[C@@H](O)c2cc(Cl)ccc2O1] | (2S,4R)-6-chloro-4-hydroxy-N-(3-{4- [cis-3-(trifluoromethoxy)cyclobutyl]- 1H-pyrazol-1-yl}bicyclo[1.1.1]pentan-1-yl)- 3,4-dihydro-2H-1-benzopyran-2- carboxamide |
| 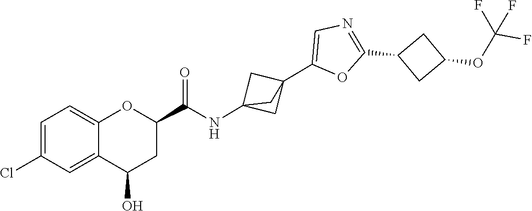 [SMILES: O=C(NC12CC(c3cnc([C@H]4C[C@@H](OC(F)(F)F)C4)o3)(C1)C2)[C@H]1C[C@@H](O)c2cc(Cl)ccc2O1] | (2R,4R)-6-chloro-4-hydroxy-N-(3-{2-[cis-3- (trifluoromethoxy)cyclobutyl]-1,3-oxazol-5- yl}bicyclo[1.1.1]pentan-1-yl)-3,4-dihydro- 2H-1-benzopyran-2-carboxamide |
| 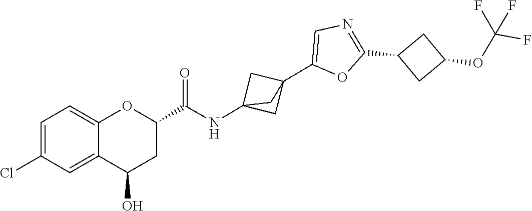 [SMILES: O=C(NC12CC(c3cnc([C@H]4C[C@@H](OC(F)(F)F)C4)o3)(C1)C2)[C@@H]1C[C@@H](O)c2cc(Cl)ccc2O1] | (2S,4R)-6-chloro-4-hydroxy-N-(3-{2- [cis-3-(trifluoromethoxy)cyclobutyl]-1,3- oxazol-5-yl}bicyclolo[1.1.1]pentan-1-yl)- 3,4-dihydro-2H-1-benzopyran-2- carboxamide |
| 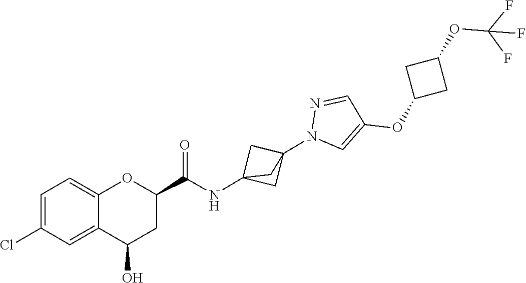 [SMILES: O=C(NC12CC(n3cc(O[C@H]4C[C@@H](OC(F)(F)F)C4)cn3)(C1)C2)[C@H]1C[C@@H](O)c2cc(Cl)ccc2O1] | (2R,4R)-6-chloro-4-hydroxy-N-[3-(4- {[cis-3-(trifluoromethoxy)cyclobutyl] oxy}-1H-pyrazol-1-yl)bicyclo[1.1.1]pentan- 1-yl]-3,4-dihydro-2H-1- benzopyran-2-carboxamide |
| 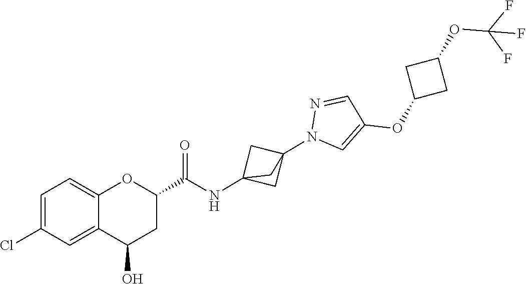 [SMILES: O=C(NC12CC(n3cc(O[C@H]4C[C@@H](OC(F)(F)F)C4)cn3)(C1)C2)[C@@H]1C[C@@H](O)c2cc(Cl)ccc2O1] | (2S,4R)-6-chloro-4-hydroxy-N-[3-(4- {[cis-3-(trifluoromethoxy)cyclobutyl]oxy}- 1H-pyrazol-1-yl)bicyclo[1.1.1]pentan-1-yl]- 3,4-dihydro-2H-1-benzopyran-2- carboxamide |
| 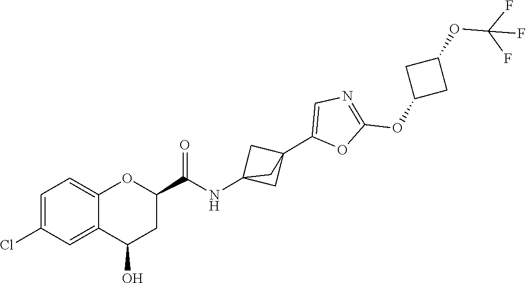 [SMILES: O=C(NC12CC(c3cnc(O[C@H]4C[C@@H](OC(F)(F)F)C4)o3)(C1)C2)[C@H]1C[C@@H](O)c2cc(Cl)ccc2O1] | (2R,4R)-6-chloro-4-hydroxy-N-[3-( 2-{[cis-3-(trifluoromethoxy)cyclobutyl] oxy}-1,3-oxazol-5-yl)bicyclo[1.1.1] pentan-1-yl]-3,4-dihydro-2H-1- benzopyran-2-carboxamide |
| 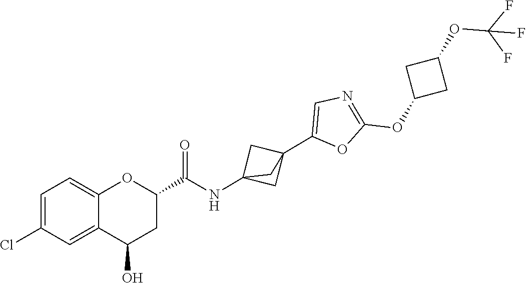 [SMILES: O=C(NC12CC(c3cnc(O[C@H]4C[C@@H](OC(F)(F)F)C4)o3)(C1)C2)[C@@H]1C[C@@H](O)c2cc(Cl)ccc2O1] | (2S,4R)-6-chloro-4-hydroxy-N-[3-(2- {[cis-3-(trifluoromethoxy)cyclobutyl]oxy}- 1,3-oxazol-5-yl)bicyclo[1.1.1]pentan-1- yl]-3,4-dihydro-2H-1- benzopyran-2-carboxamide |
| 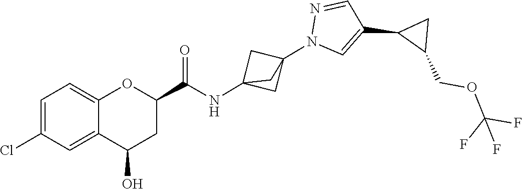 [SMILES: O=C(NC12CC(n3cc([C@H]4C[C@@H]4COC(F)(F)F)cn3)(C1)C2)[C@H]1C[C@@H](O)c2cc(Cl)ccc2O1] | (2R,4R)-6-chloro-4-hydroxy-N-[3-(4- {(1RS,2RS)-2-(trifluoromethoxy)methyl] cyclopropyl}-1H-pyrazol-1-yl)bicyclo [1.1.1]pentan-1-yl]-3,4-dihydro-2H-1- benzopyran-2-carboxamide |
| 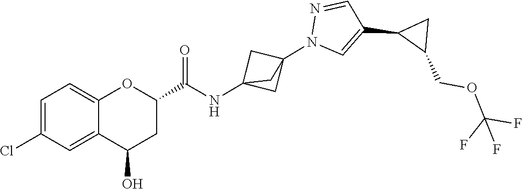 [SMILES: O=C(NC12CC(n3cc([C@H]4C[C@@H]4COC(F)(F)F)cn3)(C1)C2)[C@@H]1C[C@@H](O)c2cc(Cl)ccc2O1] | (2S,4R)-6-chloro-4-hydroxy-N-[3- (4-{(1RS,2RS)-2-[(trifluoromethoxy) methyl]cyclopropyl}-1H-pyrazol-1-yl) bicyclco[1.1.1]pentan-1-yl]-3,4-dihydro- 2H-1-benzopyran-2-carboxamide |
| 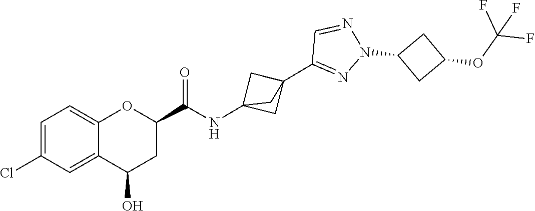 [SMILES: O=C(NC12CC(c3cnn([C@H]4C[C@@H](OC(F)(F)F)C4)n3)(C1)C2)[C@H]1C[C@@H](O)c2cc(Cl)ccc2O1] | (2R,4R)-6-chloro-4-hydroxy-N-(3-{2- [cis-3-(trifluoromethoxy)cyclobutyl]- 2H-1,2,3-triazol-4-1-yl)bicyclo [1.1.1]pentan-1-yl)-3,4-dihydro-2H-1- benzopyran-2-carboxamide |
| 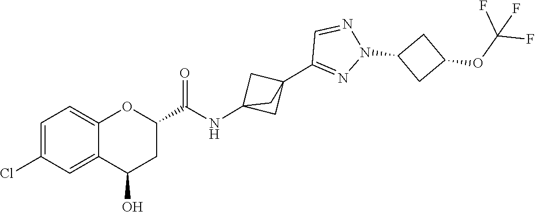 [SMILES: O=C(NC12CC(c3cnn([C@H]4C[C@@H](OC(F)(F)F)C4)n3)(C1)C2)[C@@H]1C[C@@H](O)c2cc(Cl)ccc2O1] | (2S,4R)-6-chloro-4-hydroxy-N-(3-{2- [cis-3-(trifluoromethoxy)cyclobutyl]- 2H-1,2,3-triazol-4-yl}bicyclo[1.1.1] pentan-1-yl)-3,4-dihydro-2H-1- benzopyran-2-carboxamide |
| 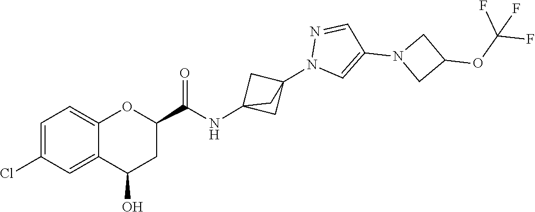 [SMILES: O=C(NC12CC(n3cc(N4CC(OC(F)(F)F)C4)cn3)(C1)C2)[C@H]1C[C@@H](O)c2cc(Cl)ccc2O1] | (2R,4R)-6-chloro-4-hydroxy-N-(3- {4-[3-(trifluoromethoxy)azetidin- 1-yl]-1H-pyrazol-1- yl}bicyclo[1.1.1]pentan- 1-yl)-3,4-dihydro-2H-1- benzopyran-2-carboxamide |
| 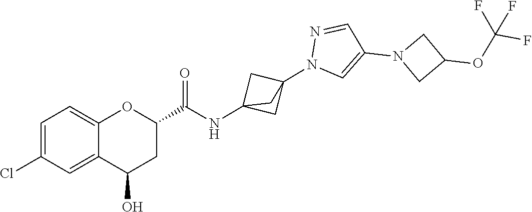 [SMILES: O=C(NC12CC(n3cc(N4CC(OC(F)(F)F)C4)cn3)(C1)C2)[C@@H]1C[C@@H](O)c2cc(Cl)ccc2O1] | (2S,4R)-6-chloro-4-hydroxy-N-(3- {4-[3-(trifluoromethoxy)azetidin- 1-yl]-1H-pyrazol-1-yl}bicyclo [1.1.1]pentan-1-yl)-3,4-dihydro- 2H-1-benzopyran-2-carboxamide |
| 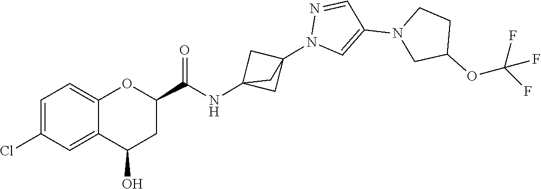 [SMILES: O=C(NC12CC(n3cc(N4CCC(OC(F)(F)F)C4)cn3)(C1)C2)[C@H]1C[C@@H](O)c2cc(Cl)ccc2O1] | (2R,4R)-6-chloro-4-hydroxy-N-(3- {4-[3-(trifluoromethoxy)pyrrolidin- 1-yl]-1H-pyrazol-1-yl}bicyclo[1.1.1] pentan-1-yl)-3,4-dihydro-2H-1- benzopyran-2-carboxamide |
| 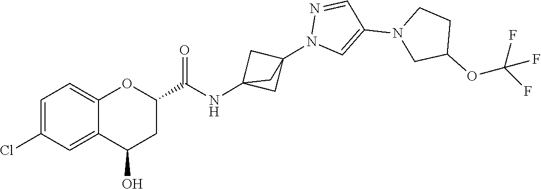 [SMILES: O=C(NC12CC(n3cc(N4CCC(OC(F)(F)F)C4)cn3)(C1)C2)[C@@H]1C[C@@H](O)c2cc(Cl)ccc2O1] | (2S,4R)-6-chloro-4-hydroxy-N-(3- {4-[3-(trifluoromethoxy)pyrrolidin-1- yl]-1H-pyrazol-1-yl}bicyclo[1.1.1] pentan-1-yl)-3,4-dihydro-2H-1- benzopyran-2-carboxamide |
| 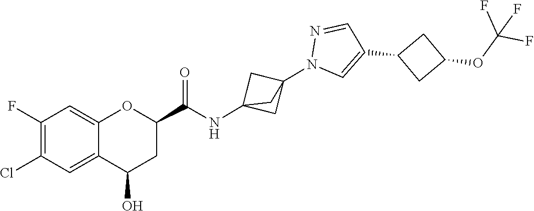 [SMILES: O=C(NC12CC(n3cc([C@H]4C[C@@H](OC(F)(F)F)C4)cn3)(C1)C2)[C@H]1C[C@@H](O)c2cc(Cl)c(F)cc2O1] | (2R,4R)-6-chloro-7-fluoro-4-hydroxy- N-(3-{4-[cis-3-(trifluoromethoxy) cyclobutyl]-1H-pyrazol-1-yl} bicyclo[1.1.1]pentan-1-yl)-3,4-dihydro- 2H-1-benzopyran-2-carboxamide |
| 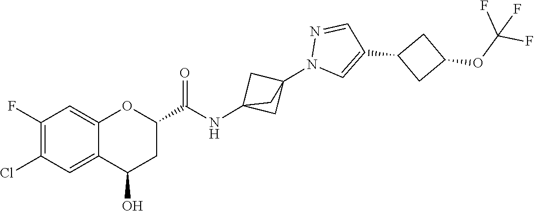 [SMILES: O=C(NC12CC(n3cc([C@H]4C[C@@H](OC(F)(F)F)C4)cn3)(C1)C2)[C@@H]1C[C@@H](O)c2cc(Cl)c(F)cc2O1] | (2S,4R)-6-chloro-7-fluoro-4-hydroxy- N-(3-{4-[cis-3-(trifluoromethoxy) cyclobutyl]-1H-pyrazol-1-yl} bicyclo[1.1.1]pentan-1-yl)-3,4-dihydro- 2H-1-benzopyran-2-carboxamide |
| 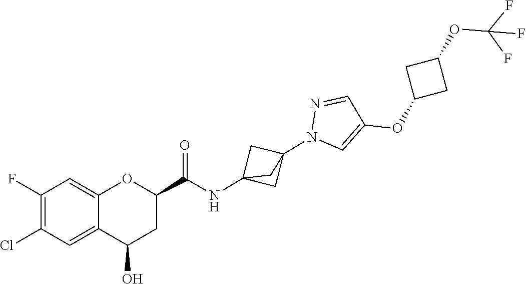 [SMILES: O=C(NC12CC(n3cc(O[C@H]4C[C@@H](OC(F)(F)F)C4)cn3)(C1)C2)[C@H]1C[C@@H](O)c2cc(Cl)c(F)cc2O1] | (2R,4R)-6-chloro-7-fluoro-4-hydroxy-N- [3-(4-{[cis-3-(trifluoromethoxy) cyclobutyl]oxy}-1H-pyrazol-1- yl)bicyclo[1.1.1]pentan-l-yl]-3,4-dihydro- 2H-1-benzopyran-2-carboxamide |
| 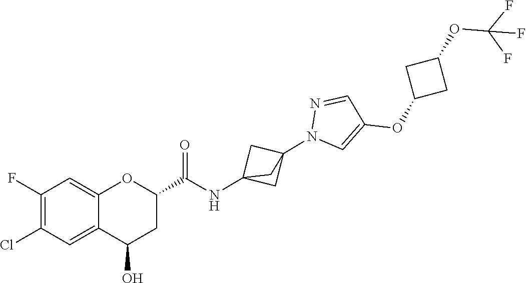 [SMILES: O=C(NC12CC(n3cc(O[C@H]4C[C@@H](OC(F)(F)F)C4)cn3)(C1)C2)[C@@H]1C[C@@H](O)c2cc(Cl)c(F)cc2O1] | (2S,4R)-6-chloro-7-fluoro-4-hydroxy- N-[3-(4-{[cis-3-(trifluoromethoxy) cyclobutyl]oxy}-1H-pyrazol-1- yl)bicyclo[1.1.1]pentan-l-yl]-3,4-dihydro- 2H-1-benzopyran-2-carboxamide |

### Example 218: Activity of Exemplary Compounds in an In Vitro Model of Vanishing Cell White Matter Disease (VWMD)

In order to test exemplary compounds of the invention in a cellular context, a stable VWMD cell line was first constructed. The ATF4 reporter was prepared by fusing the human full-length ATF4 5′-UTR (NCBI Accession No. BC022088.2) in front of the firefly luciferase (FLuc) coding sequence lacking the initiator methionine as described in Sidrauski et al (eLife 2013). The construct was used to produce recombinant retroviruses using standard methods and the resulting viral supernatant was used to transduce HEK293T cells, which were then subsequently selected with puromycin to generate a stable cell line.

HEK293T cells carrying the ATF4 luciferase reporter were plated on polylysine coated 384-well plates (Greiner Bio-one) at 30,000 cells per well. Cells were treated the next day with 1 μg/mL tunicamycin and 200 nM of a compound of Formula (I) for 7 hours. Luminescence was measured using One Glo (Promega) as specified by the manufacturer. Cells were maintained in DMEM with L-glutamine supplemented with 10% heat-inactivated FBS (Gibco) and Antibiotic-Antimycotic solution (Gibco).

Table 4 below summarizes the EC₅₀ data obtained using the ATF4-Luc assay for exemplary compounds of the invention. In this table, “A” represents an EC₅₀ of less than 10 nM; “B” an EC₅₀ greater than or equal to 10 nM and less than 50 nM; “C” an EC₅₀ greater than or equal to 50 nM and less than 250 nM; “D” an EC₅₀ greater than or equal to 250 nM and less than 500 nM; “E” an EC₅₀ greater than or equal to 500 nM and less than 2 μM; “F” an EC₅₀ of greater than 2 μM; and “G” indicates that data is not available.

TABLE 4
EC₅₀ values of exemplary compounds of the
invention in the ATF4-Luc assay.

| Compound No. | ATF4-Luc EC₅₀ |
|---|---|
| 100 | G |
| 101 | F |
| 102 | B |
| 103 | C |
| 104 | B |
| 105 | A |
| 106 | B |
| 107 | A |
| 108 | A |
| 109 | B |
| 110 | A |
| 111 | B |
| 112 | A |
| 113 | B |
| 114 | A |
| 115 | A |
| 116 | A |
| 117 | A |
| 118 | E |
| 119 | B |
| 120 | B |
| 121 | B |
| 122 | B |
| 123 | C |
| 124 | B |
| 125 | C |
| 126 | B |
| 127 | B |
| 128 | E |
| 129 | B |
| 130 | D |
| 131 | C |
| 132 | C |
| 133 | G |
| 134 | D |
| 135 | C |
| 136 | C |
| 137 | C |
| 138 | C |
| 139 | E |
| 140 | C |
| 141 | B |
| 142 | F |
| 143 | C |
| 144 | C |
| 145 | C |
| 146 | D |
| 147 | D |
| 148 | B |
| 149 | A |
| 150 | F |
| 151 | G |
| 152 | B |
| 153 | A |
| 154 | B |
| 155 | B |
| 156 | A |
| 157 | F |
| 158 | F |
| 159 | B |
| 160 | C |
| 161 | A |
| 162 | B |
| 163 | B |
| 164 | A |
| 165 | B |
| 166 | A |
| 167 | B |
| 168 | D |
| 169 | A |
| 170 | D |
| 171 | A |
| 172 | B |
| 173 | B |
| 174 | A |
| 175 | C |
| 176 | B |
| 177 | C |
| 178 | B |
| 179 | C |
| 180 | C |
| 181 | A |
| 182 | F |
| 183 | B |
| 184 | G |
| 185 | F |
| 186 | B |
| 187 | C |
| 188 | F |
| 189 | B |
| 190 | C |
| 191 | A |
| 192 | B |
| 193 | C |
| 194 | A |
| 195 | A |
| 196 | B |
| 197 | F |
| 198 | D |
| 199 | C |
| 200 | C |
| 201 | A |
| 202 | A |
| 203 | A |
| 204 | B |
| 205 | C |
| 206 | E |
| 207 | A |
| 208 | A |
| 209 | G |
| 210 | A |
| 211 | A |
| 212 | A |
| 213 | C |
| 214 | B |
| 215 | A |
| 216 | F |
| 217 | C |
| 218 | E |
| 219 | E |
| 220 | A |
| 221 | C |
| 222 | B |
| 223 | B |
| 224 | F |
| 225 | E |
| 226 | E |
| 227 | F |
| 228 | F |
| 229 | B |
| 230 | C |
| 231 | B |
| 232 | C |
| 233 | E |
| 234 | B |
| 235 | C |
| 236 | A |
| 237 | A |
| 238 | B |
| 239 | B |
| 240 | A |
| 241 | C |
| 242 | A |
| 243 | C |
| 244 | A |
| 245 | C |
| 246 | B |
| 247 | C |
| 248 | B |
| 249 | C |
| 250 | C |
| 251 | F |
| 252 | A |
| 253 | B |
| 254 | A |
| 255 | B |
| 256 | A |
| 257 | C |
| 258 | A |
| 259 | B |
| 260 | B |
| 261 | C |
| 262 | A |
| 263 | B |
| 264 | A. |
| 265 | B |
| 266 | B |
| 267 | C |
| 268 | C |
| 269 | D |
| 270 | A |
| 271 | B |
| 272 | B |
| 273 | C |
| 274 | B |
| 275 | C |
| 276 | A. |
| 277 | C |
| 278 | E |
| 279 | A |
| 280 | C |
| 281 | A |
| 282 | A |
| 283 | C |
| 284 | C |
| 285 | A |
| 286 | E |
| 287 | A |
| 288 | B |
| 289 | B |
| 290 | C |
| 291 | E |
| 292 | A |
| 293 | A |
| 294 | B |
| 295 | B |
| 296 | E |
| 297 | B |
| 298 | C |
| 299 | E |
| 300 | C |
| 301 | E |
| 302 | A |
| 303 | C |
| 304 | A |
| 305 | A |
| 306 | G |
| 307 | A |
| 308 | A |
| 309 | F |
| 310 | C |
| 311 | G |
| 312 | G |
| 313 | D |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A compound of Formula (I): 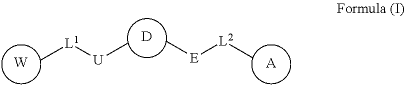
or a pharmaceutically acceptable salt, solvate, hydrate, tautomer, N-oxide, or stereoisomer thereof, wherein:
D is a bridged bicyclic cycloalkyl, a bridged bicyclic heterocyclyl, a 4-6-membered monocyclic cycloalkyl, a 4-6-membered monocyclic heterocyclyl, or cubanyl, wherein each bridged bicyclic cycloalkyl, bridged bicyclic heterocyclyl, 4-6-membered monocyclic cycloalkyl, 4-6-membered monocyclic heterocyclyl, or cubanyl is optionally substituted on one or more available carbons with 1-4 R^{X}; and wherein if the 4-6-membered monocyclic heterocyclyl or bridged bicyclic heterocyclyl contains a substitutable nitrogen moiety, the substitutable nitrogen may be optionally substituted by R^{N1};
U is —NR¹C(O)—, —C(O)NR¹— or 5-6-membered heteroaryl;
E is a bond, —NR²C(O)—, —C(O)NR²—, 5-6-membered heteroaryl or 5-6-membered heterocyclyl; wherein 5-6-membered heteroaryl or 5-6-membered heterocyclyl is optionally substituted on one or more available carbons with 1-5 R^{G}; and wherein if the 5-6-membered heteroaryl or 5-6-membered heterocyclyl contains a substitutable nitrogen moiety, the substitutable nitrogen may be optionally substituted by R^{N2}; or
E is 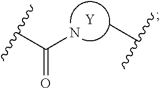
Y is a 4-9-membered nitrogen-containing monocyclic, bridged bicyclic, fused bicyclic or spirocyclic heterocyclyl, wherein the 4-9-membered nitrogen-containing monocyclic, bridged bicyclic, fused bicyclic or spirocyclic heterocyclyl is optionally substituted on one or more available carbons with 1-5 R^{G}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2};
L¹ is a bond, C₁-C₆ alkylene, 2-7 membered heteroalkylene, —NR^{N3}—, or —O—, wherein C₁-C₆ alkylene or 2-7 membered heteroalkylene is optionally substituted with 1-5 R^{L1};
L² is a bond, C₁-C₆ alkylene, 2-7 membered heteroalkylene, or —O—, wherein C₁-C₆ alkylene or 2-7 membered heteroalkylene is optionally substituted with 1-5 R^{L2};
R¹ is hydrogen or C₁-C₆ alkyl;
R² is hydrogen or C₁-C₆ alkyl;
W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available carbons with 1-4 R^{W1}; wherein the phenyl or heteroaryl may optionally be substituted on one or more available unsaturated carbons with 1-4 R^{W2}; wherein if the heterocyclyl contains a substitutable nitrogen moiety, the substitutable nitrogen may optionally be substituted with R^{N4}; and wherein W is attached to L² through an available saturated carbon or nitrogen atom within the heterocyclyl;
A is C₃-C₆ cycloalkyl, phenyl, 4-6-membered heterocyclyl, 5-6-membered heteroaryl, or 8-10-membered bicyclic heteroaryl, wherein C₃-C₆ cycloalkyl, phenyl, 4-6-membered heterocyclyl, 5-6-membered heteroaryl, or 8-10-membered bicyclic heteroaryl is optionally substituted on one or more available carbons with 1-5 R^{Y}; and wherein if the 5-6-membered heteroaryl or 8-10-membered bicyclic heteroaryl contains a substitutable nitrogen moiety, the substitutable nitrogen may be optionally substituted by R^{N5};
each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N3} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N4} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B}R^{C} and —C(O)OR^{D};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O)₂—NR^{B}R^{C};
R^{N5} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A}, —NR^{B}R^{C}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)R^{D};
each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, halo-C₁-C₆ alkoxy-C₁-C₆ alkyl, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or
2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};
each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo;
each R^{G} is independently hydrogen, C₁-C₆ alkyl, halo or oxo; and
m is 1 when R^{F} is hydrogen or C₁-C₆ alkyl, 3 when R^{F} is C₁-C₆ alkyl, or 5 when R^{F} is halo.

2. The compound of claim 2, wherein D is bicyclo[1.1.1]pentane, bicyclo[2.2.1]heptane, bicyclo[2.1.1]hexane, bicyclo[2.2.2]octane, bicyclo[3.2.1]octane, 2-oxabicyclo[2.2.2]octane, 7-oxabicyclo[2.2.1]heptane, 8-azabicyclo[3.2.1]octane, cyclohexyl or tetrahydro-2H-pyranyl, each of which is optionally substituted with 1-4 R^{X} groups.

3. The compound of claims 1-2, wherein D is selected from the group consisting of

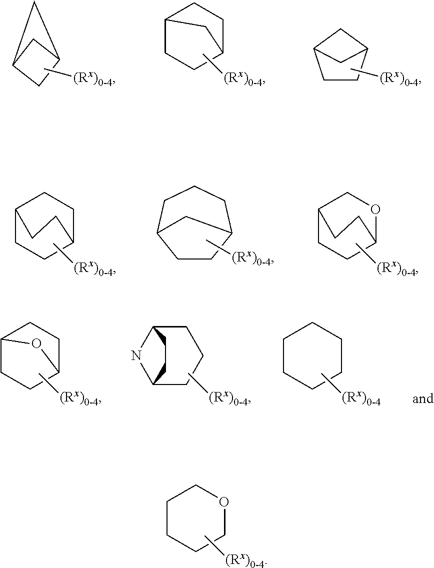

4. The compound of an one of claims 1-3, wherein D is selected from the group consisting of

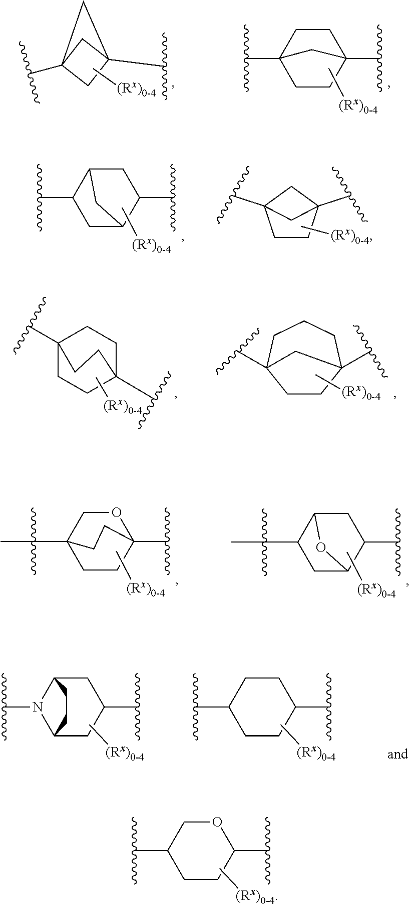

5. The compound of any one of claims 1-4, wherein D is substituted with 0 R^{X}.

6. The compound of any one of claims 1-5, wherein D is selected from the group consisting of

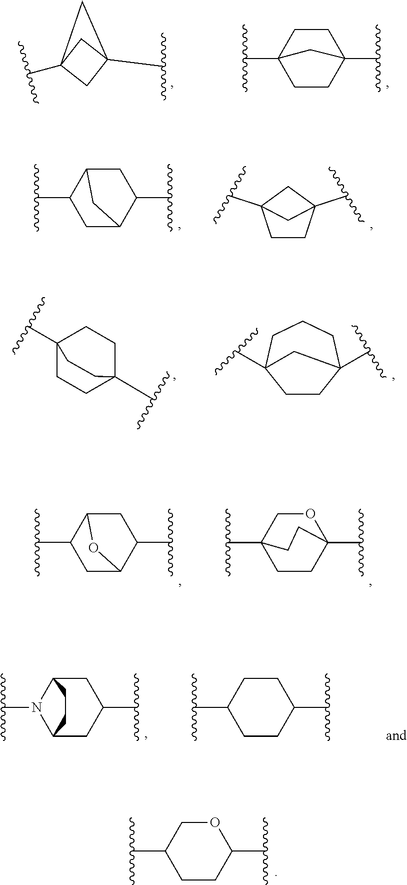

7. The compound of any one of claims 1-6, wherein D is

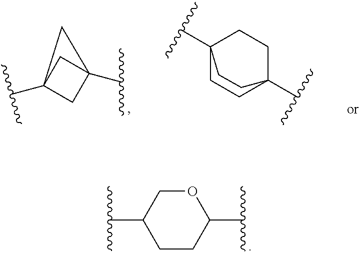 [SMILES: *C12CC(*)(C1)C2 ; *C12CCC(*)(CC1)CC2 ; *C1CCC(*)OC1]

8. The compound of any one of claims 1-5, wherein D is substituted with 1 R^{X}.

9. The compound of any one of claims 1-5 and 8, wherein D is

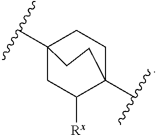

10. The compound of claim 8 or 9, wherein R^{X} is —OH.

11. The compound of any one of claims 1-10, wherein U is selected from the group consisting of —NHC(O)—, —C(O)NH— and

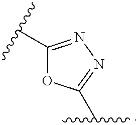 [SMILES: *c1nnc(*)o1]

12. The compound of any one of claims 1-11, wherein U is —NHC(O)—.

13. The compound of anyone of claims 1-12, wherein L¹ is a bond or C₁-C₆ alkylene, wherein C₁-C₆ alkylene is optionally substituted with 1-5 R^{L1}.

14. The compound of any one of claims 1-13, wherein L¹ is a bond or C₁-C₆ alkylene, wherein C₁-C₆ alkylene is substituted with 0 R^{L1}.

15. The compound of any one of claims 1-14, wherein L¹ is a bond or —CH₂—.

16. The compound of any one of claims 1-15, wherein R¹ is hydrogen or CH₃.

17. The compound of any one of claims 1-16, wherein W is represented by Formula (W-a): 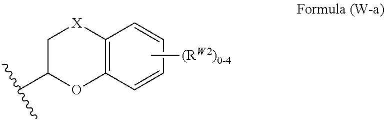
wherein:
X is NR^{N4} or C(R^{X1})(R^{X2});
R^{N4} is hydrogen or C₁-C₆ alkyl;
R^{X1} is hydrogen or hydroxyl;
R^{X2} is hydrogen or hydroxyl; or
R^{X1} and R^{X2} taken together to form an oxo moiety.

18. The compound of an one of claims 1-17 wherein W is selected from the group consisting of

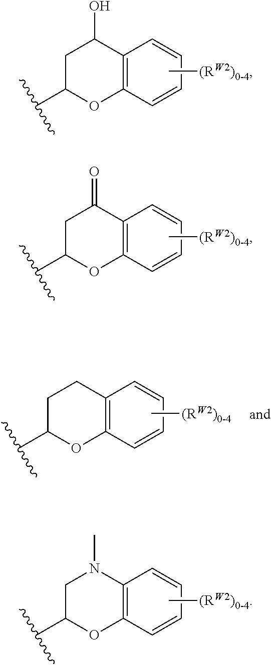

19. The compound of any one of claims 1-16, wherein W is

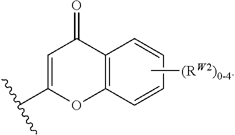

20. The compound of any one of claims 1-19, wherein W is substituted with 1 R^{W2}.

21. The compound of claim 20, wherein R^{W2} is chloro.

22. The compound of any one of claims 1-19, wherein W is substituted with 2 R^{W2}.

23. The compound of claim 22, wherein each R^{W2} is independently chloro or fluoro.

24. The compound of any one of claims 1-23, wherein E is selected from the group consisting of a bond, —NR²C(O)—, —C(O)NR²—, and

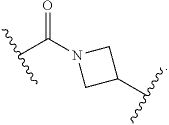 [SMILES: *C(=O)N1CC(*)C1]

25. The compound of any one of claims 1-21, wherein E is selected from the group consisting of

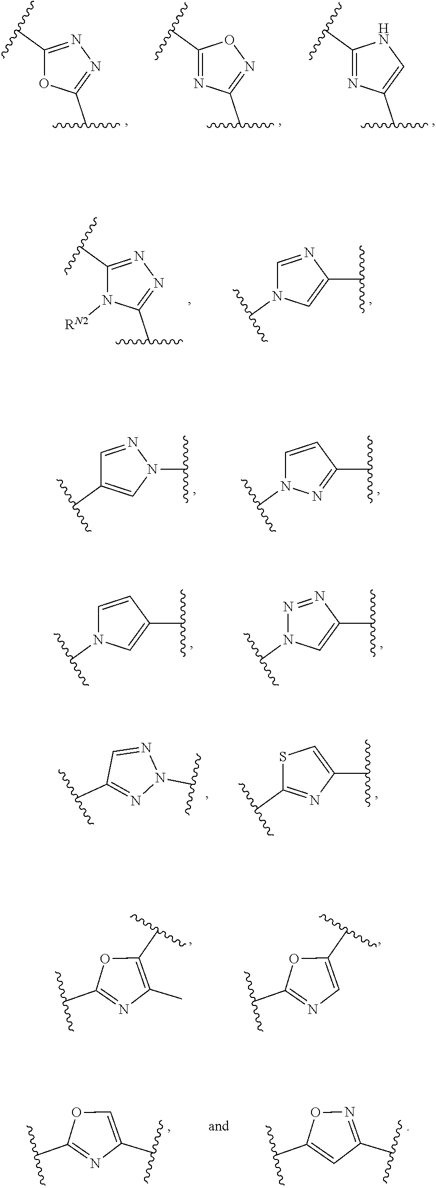

26. The compound of any one of claims 1-21, wherein E is selected from the group consisting of

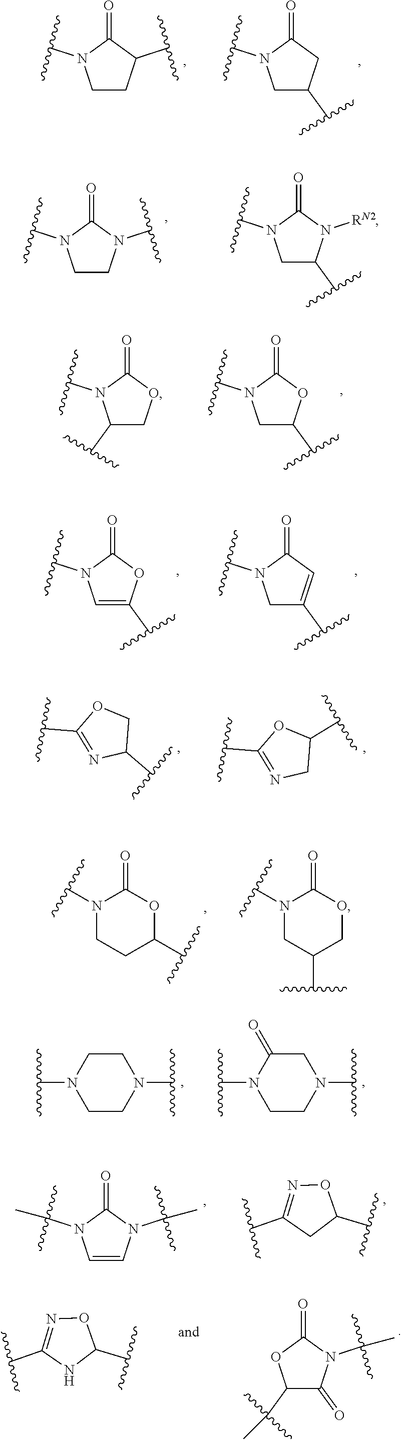

27. The compound of any one of claims 1-26, wherein E is selected from the group consisting of a bond, —NR²C(O)—, —CONR²—,

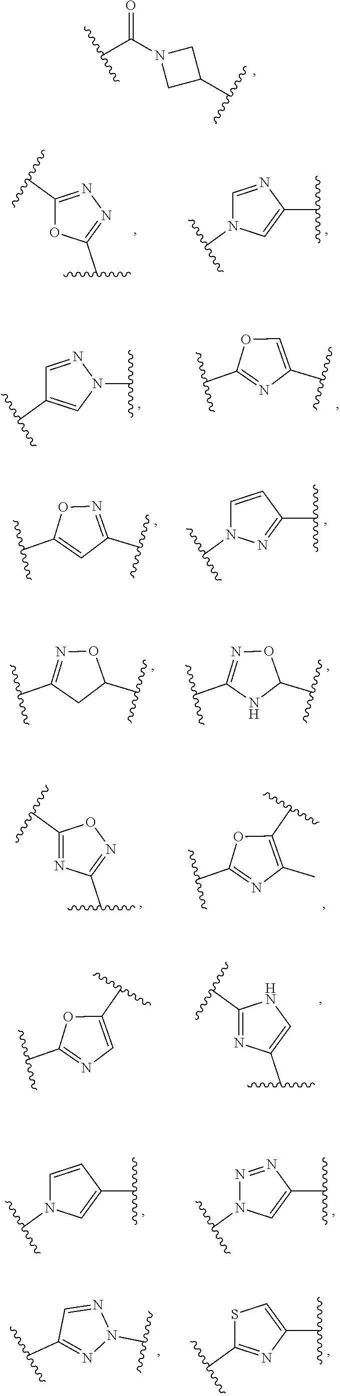

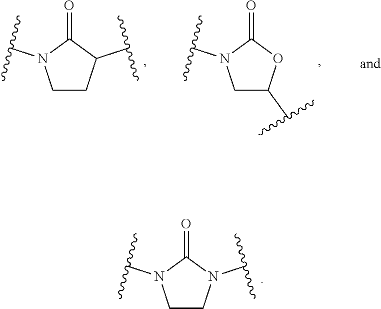 [SMILES: *C1CCN(*)C1=O ; *C1CN(*)C(=O)O1 ; *N1CCN(*)C1=O]

28. The compound of any one of claims 1-17, wherein E is selected from the group consisting of

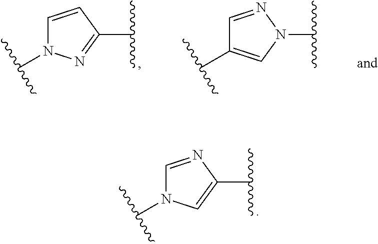 [SMILES: *c1ccn(*)n1 ; *c1cnn(*)c1 ; *c1cn(*)cn1]

29. The compound of any one of claims 1-28, wherein R² is hydrogen.

30. The compound of any one of claims 1-29, wherein L² is a bond, —O—, C₁-C₆ alkylene, or 2-7 membered heteroalkylene.

31. The compound of any one of claims 1-30, wherein L² is a bond, —CH₂—, —CH₂O—*, —(CH₂)₂O—*, —(CH₂)₃O—*, or —O—, wherein “—*” indicates the attachment point to A.

32. The compound of any one of claims 1-31, wherein A is selected from the group consisting of:

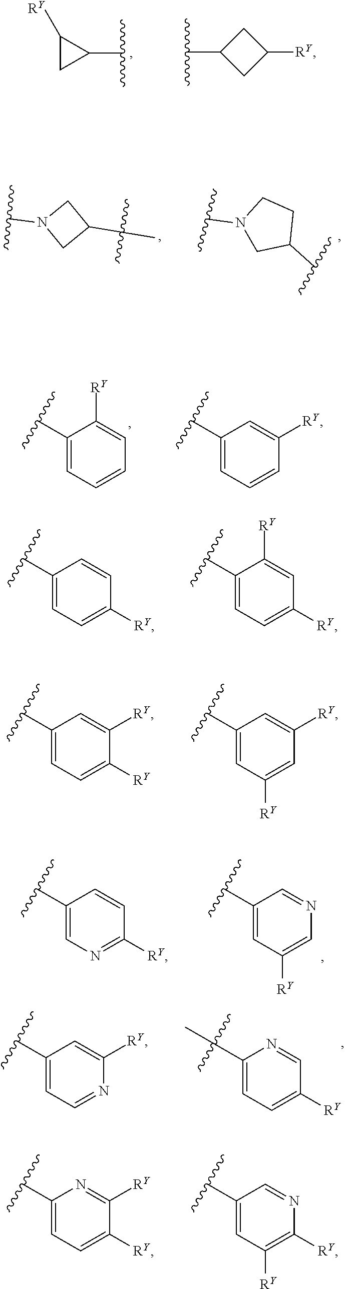

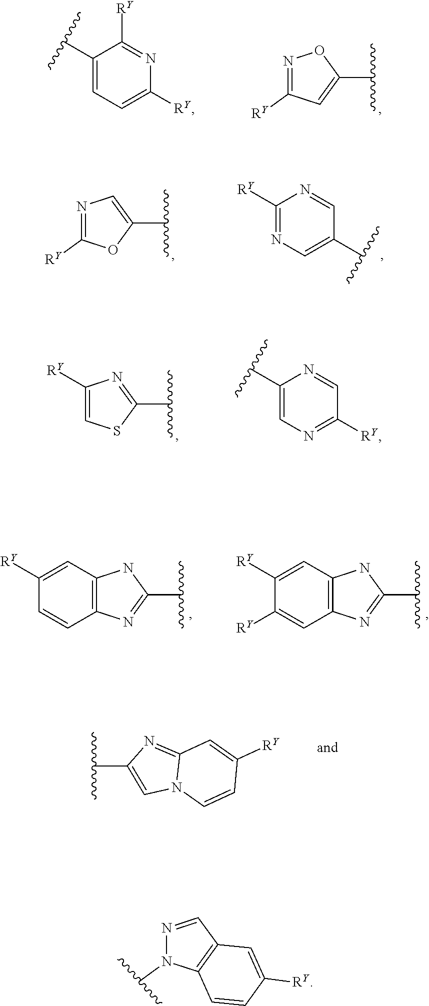

33. The compound of any one of claims 1-32, wherein A is selected from the group consisting of:

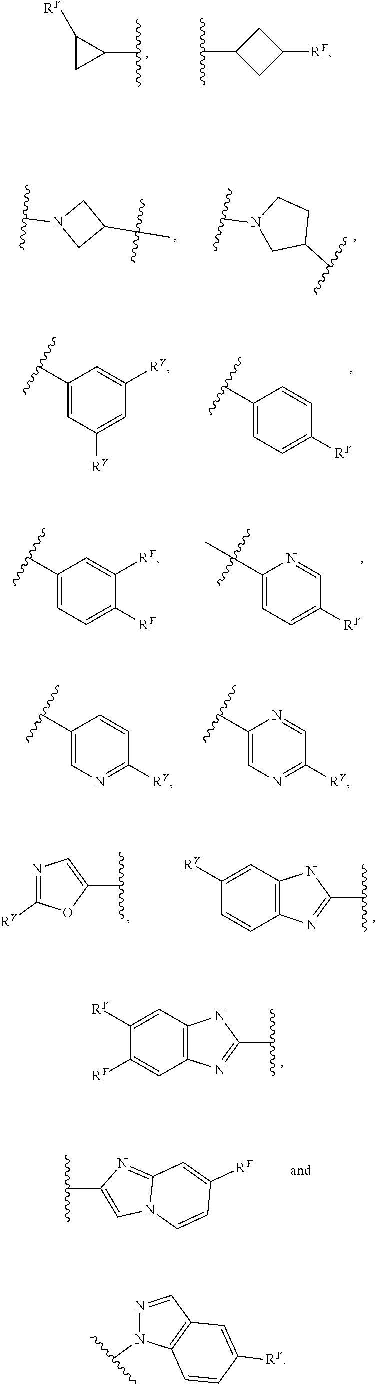

34. The compound of any one of claims 1-33, wherein each R^{Y} is independently selected from the group consisting of hydrogen, chloro, fluoro, hydroxyl, phenyl, CHF₂, CF₃, CH₃, CH₂CH₃, CH(CH₃)₂, OCH₃, OCHF₂, OCF₃, OCH₂CF₃, OCH(CH₃)₂, CH₂OCF₃, and CN.

35. A compound of Formula (II): 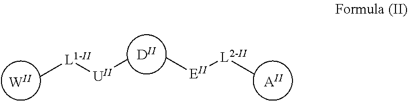
or a pharmaceutically acceptable salt, solvate, hydrate, tautomer, N-oxide, or stereoisomer thereof, wherein:
D^{II} is a bridged bicyclic cycloalkyl, a bridged bicyclic heterocyclyl, a 4-6-membered monocyclic cycloalkyl, a 4-6-membered monocyclic heterocyclyl, or cubanyl, wherein each bridged bicyclic cycloalkyl, bridged bicyclic heterocyclyl, 4-6-membered monocyclic cycloalkyl, 4-6-membered monocyclic heterocyclyl, or cubanyl is optionally substituted on one or more available carbons with 1-4 R^{X-II}; and wherein if the 4-6-membered monocyclic heterocyclyl or bridged bicyclic heterocyclyl contains a substitutable nitrogen moiety, the substitutable nitrogen may be optionally substituted by R^{N1-II};
U^{II} is —NR^{1-II}C(O)— or —C(O)NR^{1-II}—;
E^{II} is a bond, —NR^{2-II}C(O)—, —C(O)NR^{2-II}—, 5-6-membered heteroaryl or 5-6-membered heterocyclyl; wherein 5-6-membered heteroaryl or 5-6-membered heterocyclyl is optionally substituted on one or more available carbons with 1-5 R^{G-II}; and wherein if the 5-6-membered heteroaryl or 5-6-membered heterocyclyl contains a substitutable nitrogen moiety, the substitutable nitrogen may be optionally substituted by R^{N2-II}; or
E^{II} is 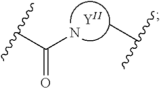
Y^{II} is a 4-9-membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{G-II}; and wherein if the 4-9-membered nitrogen-containing monocyclic, bridged bicyclic, fused bicyclic or spirocyclic heterocyclyl contains a substitutable nitrogen moiety, the substitutable nitrogen may be optionally substituted by R^{N2-II};
L^{1-II} is a bond, C₁-C₆ alkylene, 2-7 membered heteroalkylene, —NR^{N3-II}—, or —O—, wherein C₁-C₆ alkylene or 2-7 membered heteroalkylene is optionally substituted with 1-5 R^{L1-II};
L^{2-II} is a bond, C₁-C₆ alkylene, or 2-7 membered heteroalkylene, —O—, wherein C₁-C₆ alkylene or 2-7 membered heteroalkylene is optionally substituted with 1-5 R^{L2-II};
R^{1-II} is hydrogen or C₁-C₆ alkyl;
R^{2-II} is hydrogen or C₁-C₆ alkyl;
W^{II} is phenyl or 5-6-membered heteroaryl; wherein phenyl or 5-6-membered heteroaryl is optionally substituted with 1-5 R^{W-II}; and wherein if the 5-6-membered heteroaryl contains a substitutable nitrogen moiety, the substitutable nitrogen may be optionally substituted by R^{N4-II};
A^{II} is C₃-C₆ cycloalkyl, phenyl, or 5-6-membered heteroaryl, wherein C₃-C₆ cycloalkyl, phenyl, or 5-6-membered heteroaryl is optionally substituted on one or more available carbons with 1-5 R^{Y-II}; and wherein if the 5-6-membered heteroaryl contains a substitutable nitrogen moiety, the substitutable nitrogen may be optionally substituted by R^{N5-II};
each R^{L1-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};
each R^{L2-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};
R^{N1-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};
R^{N2-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};
R^{N3-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};
R^{N4-II} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S(O)₂—C₁-C₆ alkyl, —S(O)₂-phenyl, —S(O)₂-heteroaryl, —C(O)NR^{B-II}R^{C-II} and —C(O)OR^{D-II};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w-II}C₁-C₆ alkyl (wherein w-II is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), and S(O₂)NR^{B-II}R^{C-II};
R^{N5-II} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-II}R^{C-II}, —C(O)R^{D-II}, —C(O)OR^{D-II}, and —S(O)₂R^{D-II};
each R^{W-II} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, oxo, C═N—OH, halo, cyano, —OR^{A-II}, —NR^{B-II}R^{C-II}, —NR^{B-II}R^{CC-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II}; or
2 R^{W-II} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-II};
each R^{X-II} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —SR^{E-II}, —S(O)R^{D-II}, and —S(O)₂R^{D-II};
each R^{Y-II} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, —S(R^{F-II})_{m-II}, —S(O)R^{D-II}, —S(O)₂R^{D-II}, and G^{1-II}; or
2 R^{Y-II} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-II};
each G^{1-II} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-II};
each R^{Z-II} is independently selected from the group consisting of C₁-C₆ alkyl, hydroxy-C₁-C₆ alkyl, halo-C₁-C₆ alkyl, halo, cyano, —OR^{A-II}, —NR^{B-II}R^{C-II}, —NR^{B-II}C(O)R^{D-II}, —C(O)NR^{B-II}R^{C-II}, —C(O)R^{D-II}, —C(O)OH, —C(O)OR^{D-II}, and —S(O)₂R^{D-II};
R^{A-II} is, at each occurrence, independently hydrogen, C₁-C₆ alkyl, halo-C₁-C₆ alkyl, —C(O)NR^{B-II}R^{C-II}, —C(O)R^{D-II}, or —C(O)OR^{D-II};
each of R^{B-II} and R^{C-II} is independently hydrogen or C₁-C₆ alkyl;
R^{B-II} and R^{C-II} together with the atom to which they are attached form a 3-7-membered heterocyclyl ring optionally substituted with 1-3 R^{Z-II};
each R^{CC-II} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D-II} is independently C₁-C₆ alkyl or halo-C₁-C₆ alkyl;
each R^{E-II} is independently hydrogen, C₁-C₆ alkyl, or halo-C₁-C₆ alkyl;
each R^{F-II} is independently hydrogen, C₁-C₆ alkyl, or halo; and
each R^{G-II} is independently hydrogen, C₁-C₆ alkyl, halo or oxo;
provided that when D^{II} is a bridged bicyclic 5-membered cycloalkyl, E^{II} is —NR^{2-II}C(O)—.

36. The compound of claim 35, wherein D^{II} is bicyclo[1.1.1]pentane, bicyclo[2.2.1]heptane, bicyclo[2.1.1]hexane, bicyclo[2.2.2]octane, bicyclo[3.2.1]octane, 7-oxabicyclo[2.2.1]heptane, 8-azabicyclo[3.2.1]octane, cyclohexyl or tetrahydro-2H-pyranyl, each of which is optionally substituted with 1-4 R^{X-II} groups.

37. The compound of claims 35-36, wherein D^{II} is selected from the group consisting of

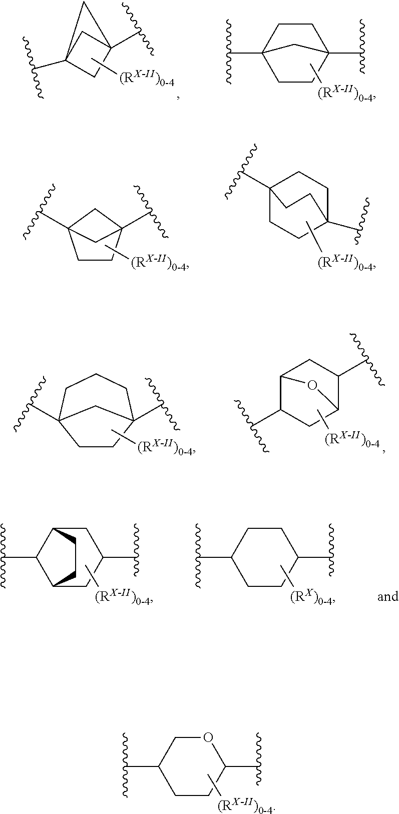

38. The compound of any one of claims 35-37, wherein D^{II} is substituted with 0 R^{X-II}.

39. The compound of any one of claims 35-38, wherein D^{II} is selected from the group consisting of

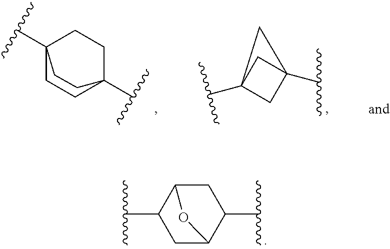 [SMILES: *C12CCC(*)(CC1)CC2 ; *C12CC(*)(C1)C2 ; *C1CC2OC1CC2*]

40. The compound of any one of claims 35-37, wherein D^{II} is substituted with 1 R^{X-II}.

41. The compound of any one of claims 35-37 and 40, wherein D^{II} is

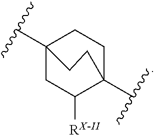

42. The compound of claim 40 or 41, wherein R^{X-II} is —OH.

43. The compound of any one of claims 35-42, wherein L^{1-II} is a C₁-C₆ alkylene or a 2-7 membered heteroalkylene, wherein the C₁-C₆ alkylene or 2-7 membered heteroalkylene is optionally substituted with 1-5 R^{L1-II}.

44. The compound of any one of claims 35-43, wherein L^{1-II} is a C₁-C₆ alkylene or a 2-7 membered heteroalkylene substituted with 0 R^{L1-II}.

45. The compound of any one of claims 35-44, wherein L^{1-II} is —CH₂— or CH₂O—*, wherein “—*” indicates the attachment point to W^{II}.

46. The compound of any one of claims 35-45, wherein R^{1-II} is hydrogen or CH₃.

47. The compound of any one of claims 35-46, wherein W^{II} is selected from the group

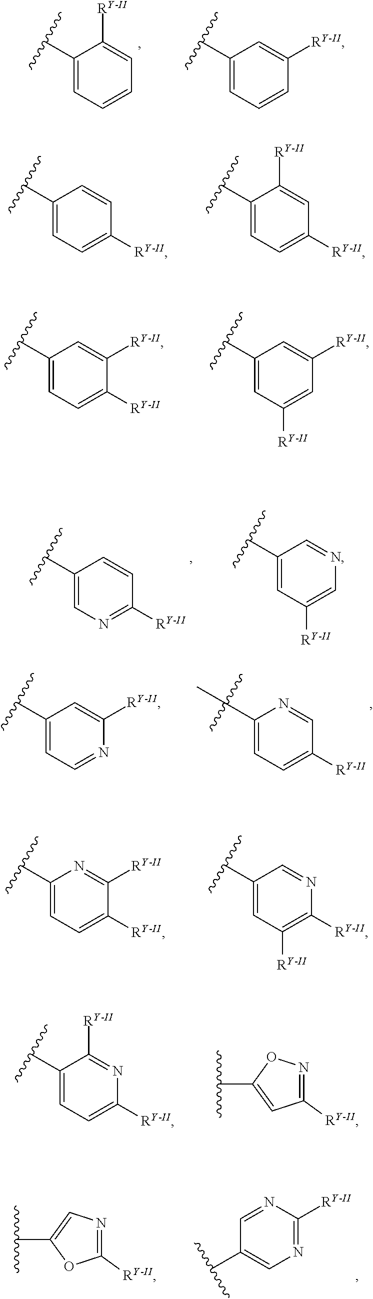

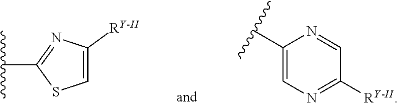

48. The compound of any one of claims 35-47, wherein W^{II} is

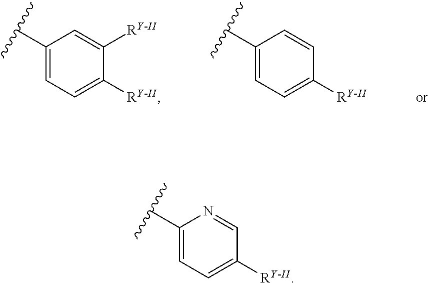

49. The compound of any one of claims 35-48, wherein each R^{Y-II} is independently chloro, fluoro or CF₃.

50. The compound of any one of claims 35-49, wherein E^{II} is selected from the group consisting of —NR^{2-II}C(O)—, —C(O)NR^{2-II}, and

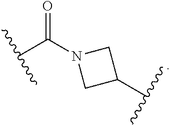 [SMILES: *C(=O)N1CC(*)C1]

51. The compound of any one of claims 35-49, wherein E^{II} is selected from the group consisting of

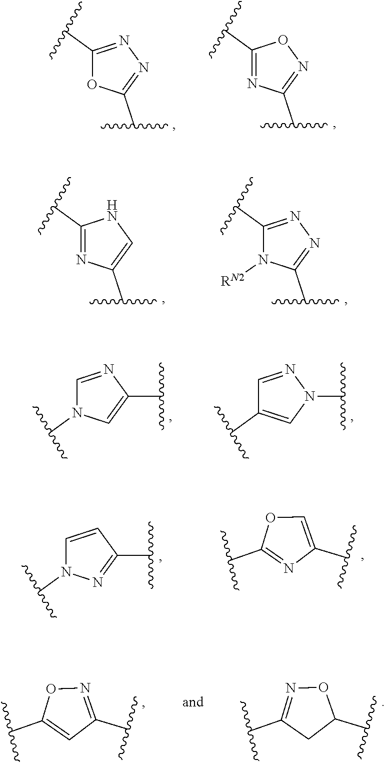

52. The compound of any one of claims 35-51, wherein E^{II} is selected from the group consisting of —NR^{2-II}C(O)—,

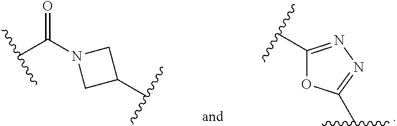 [SMILES: *C(=O)N1CC(*)C1 ; *c1nnc(*)o1]

53. The compound of any one of claims 35-50 and 52, wherein E^{II} is —NR^{2-II}C(O)— when D^{II} is

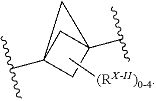

54. The compound of any one of claims 35-53, wherein R^{2-II} is hydrogen or methyl.

55. The compound of any one of claims 35-54, wherein L^{2-II} is a bond, —O—, or 2-7 membered heteroalkylene.

56. The compound of any one of claims 35-55, wherein L^{2-II} is a bond, —CH₂O—*, —(CH₂)₂O—*, —(CH₂)₃O—*, or —O—, wherein “—*” indicates the attachment point to A^{II}.

57. The compound of any one of claims 35-56, wherein A^{II} is selected from the group consisting of:

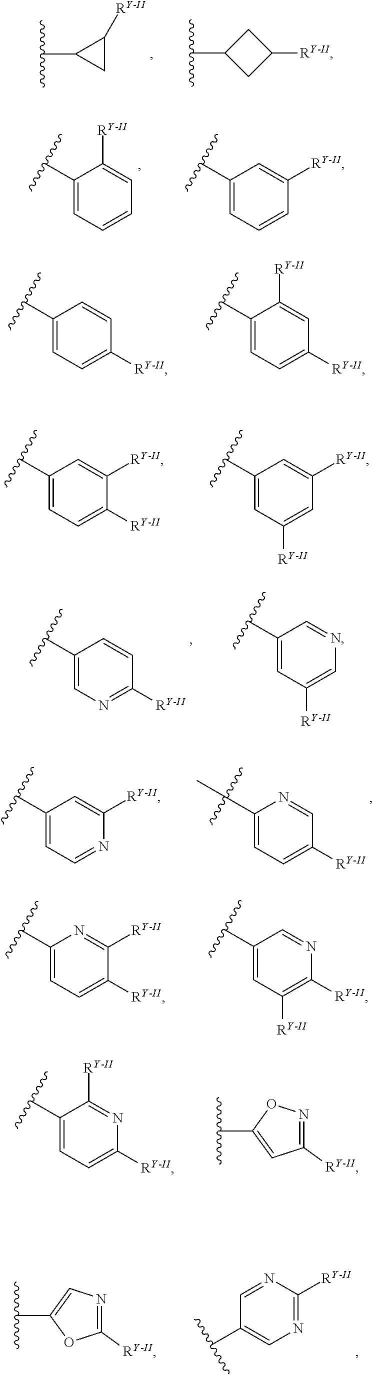

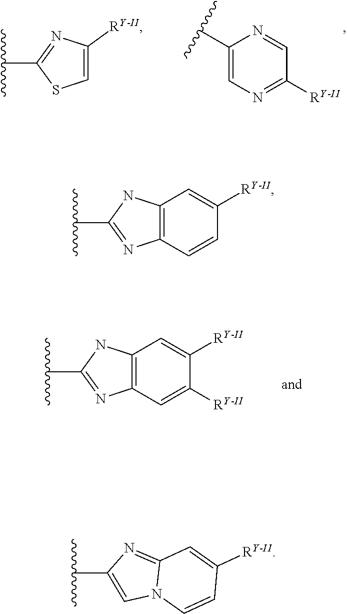

58. The compound of any one of claims 35-57, wherein A^{II} is, or

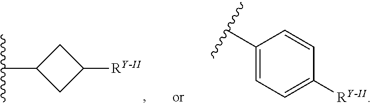

59. The compound of any one of claims 35-58, wherein each R^{Y-II} is chloro or OCF₃.

60. A compound represented by Formula (IIIa) or Formula (IIIb): 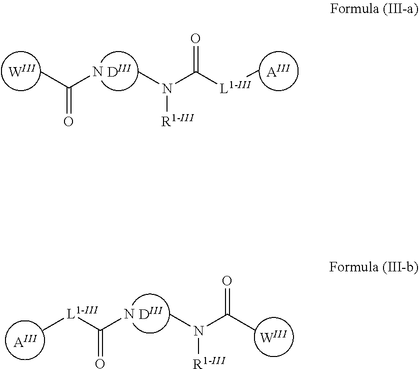
or a pharmaceutically acceptable salt, solvate, hydrate, tautomer, N-oxide, or stereoisomer thereof, wherein:
D^{III} is a 4-9 membered nitrogen-containing monocyclic, bridged bicyclic, fused bicyclic or spirocyclic heterocyclyl, wherein the 4-9 membered monocyclic, bridged bicyclic, fused bicyclic or spirocyclic heterocyclyl is optionally substituted on one or more available carbons with 1-5 R^{X-III}; and wherein if the 4-9 membered nitrogen-containing monocyclic, bridged bicyclic, fused bicyclic or spirocyclic heterocyclyl contains a substitutable nitrogen moiety, the substitutable nitrogen may be optionally substituted by R^{N1-III};
W^{III} is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available saturated carbons with 1-4 R^{W1-III}; wherein the phenyl or heteroaryl may optionally be substituted on one or more available unsaturated carbons with 1-4 R^{W2-III}; and wherein if the heterocyclyl contains a substitutable nitrogen moiety, the substitutable nitrogen may optionally be substituted with R^{N2-III};
A^{III} is phenyl or 5-6-membered heteroaryl, wherein phenyl or 5-6-membered heteroaryl is optionally substituted on one or more available carbons with 1-5 R^{Y-III}; and
wherein if the 5-6-membered heteroaryl contains a substitutable nitrogen moiety, the substitutable nitrogen may be optionally substituted by R^{N3-III};
R^{1-III} is hydrogen or C₁-C₆ alkyl;
L^{1-III} is a bond, C₁-C₆ alkylene or 2-7 membered heteroalkylene, wherein C₁-C₆ alkylene or 2-7 membered heteroalkylene is optionally substituted with 1-5 R^{L1-III};
each R^{L1-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};
R^{N1-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};
R^{N2-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};
R^{N3-III} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B-III}R^{C-III}, —C(O)R^{D-III}, —C(O)OR^{D-III}, and —S(O)₂R^{D-III};
each R^{W1-III} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═N—OH, halo, cyano, —OR^{A-III}, —NR^{B-III}R^{C-III}, —NR^{B-II}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};
each R^{W2-III} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}U, —C(O)OH, —C(O)OR^{D-III}, —S(R^{F-III})_{m-III}, —S(O)R^{D-III}, and —S(O)₂R^{D-III}; or
2 R^{W2-III} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X-III};
each R^{X-III} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —SR^{E-III}, —S(O)R^{D-III}, and —S(O)₂R^{D-III};
each R^{Y-III} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, —S(R^{F-III})_{m-III}, —S(O)R^{D-III}, —S(O)₂R^{D-III}, and G^{1-III}; or
2 R^{Y-III} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X-III};
each G^{1-III} is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z-III};
each R^{Z-III} is independently selected from the group consisting of C₁-C₆ alkyl, hydroxy-C₁-C₆ alkyl, halo-C₁-C₆ alkyl, halo, cyano, —OR^{A-III}, —NR^{B-III}R^{C-III}, —NR^{B-III}C(O)R^{D-III}, —C(O)NR^{B-III}R^{C-III}, —C(O)R^{D-III}, —C(O)OH, —C(O)OR^{D-III}, and —S(O)₂R^{D-III};
R^{A-III} is, at each occurrence, independently hydrogen, C₁-C₆ alkyl, halo-C₁-C₆ alkyl, —C(O)NR^{B-III}R^{C-III}, —C(O)R^{D-III}, or —C(O)OR^{D-III};
each of R^{B-III} and R^{C-III} is independently hydrogen or C₁-C₆ alkyl; or
R^{B-III} and R^{C-III} together with the atom to which they are attached form a 3-7-membered heterocyclyl ring optionally substituted with 1-3 R^{Z-III};
each R^{CC-III} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D-III} is independently C₁-C₆ alkyl, hydroxy-C₁-C₆, alkyl, or halo-C₁-C₆ alkyl;
each R^{E-III} is independently hydrogen, C₁-C₆ alkyl, or halo-C₁-C₆ alkyl;
each R^{F-III} is independently hydrogen, C₁-C₆ alkyl, or halo; and
m^{III} is 1 when R^{F-II} is hydrogen or C₁-C₆ alkyl, 3 when R^{F-III} is C₁-C₆ alkyl, or 5 when R^{F-III} is halo.

61. The compound of claim 60, wherein D^{III} is an azetidine, pyrrolidine, piperidine, piperazine, or 2-azaspiro[3.3]heptane moiety, each of which is optionally substituted with 1-4 R^{W-III} groups, and each R^{W-III} is independently C₁-C₆ alkyl, halo-C₁-C₆ alkyl, halo, oxo, cyano, or —OR^{A-III}, and wherein piperazine is optionally substituted on a substitutable nitrogen by R^{N2-III}.

62. The compound of claim 60 or 61, wherein D^{III} is selected from the group consisting of: 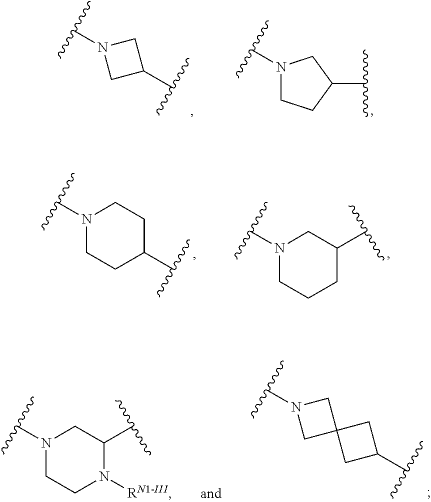
wherein R^{N1-III} is hydrogen or C₁-C₃ alkyl.

63. The compound of claim 62, wherein D^{III} is

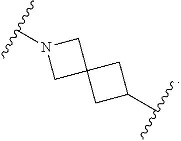 [SMILES: *C1CC2(C1)CN(*)C2]

64. The compound of any one of claims 60-63, wherein W^{III} is represented by Formula (W-b): 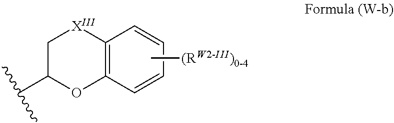
wherein:
X^{III} is NR^{N4-III} or C(R^{X1-III})(R^{X2-III});
R^{N4-III} is hydrogen or C₁-C₆ alkyl;
R^{X1-III} is hydrogen or hydroxyl;
R^{X2-III} is hydrogen or hydroxyl; or
R^{X1-III} and R^{X2-III} taken together to form an oxo moiety.

65. The compound of any one of claims 60-64, wherein W^{III} is selected from the group consisting of

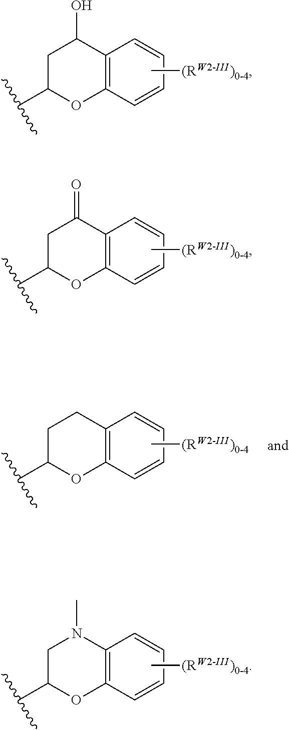

66. The compound of any one of claims 60-65, wherein W^{III} is substituted with 1 R^{W2-III}.

67. The compound of claim 66, wherein R^{W2-III} is chloro.

68. The compound of any one of claims 60-67, wherein L^{1-III} is 2-7 membered heteroalkylene optionally substituted by 1-5 R^{L1-III}.

69. The compound of any one of claims 60-68, wherein L^{1-III} is 2-7 membered heteroalkylene substituted by 0 R^{L1}.

70. The compound of any one of claims 60-98, wherein L^{1-III} is selected from CH₂O—* or CH₂OCH₂—*, wherein “—*” indicates the attachment point to A^{III}.

71. The compound of any one of claims 60-70, wherein R^{1-III} is hydrogen or CH₃.

72. The compound of any one of claims 60-71, wherein A^{III} is selected from the group consisting of:

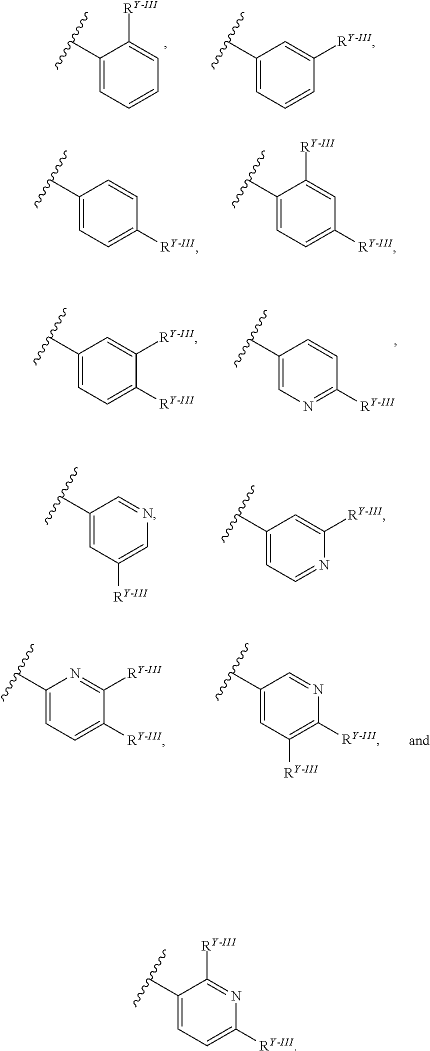

73. The compound of any one of claims 60-72, wherein each R^{Y-III} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN.

74. A compound selected from the group consisting of: 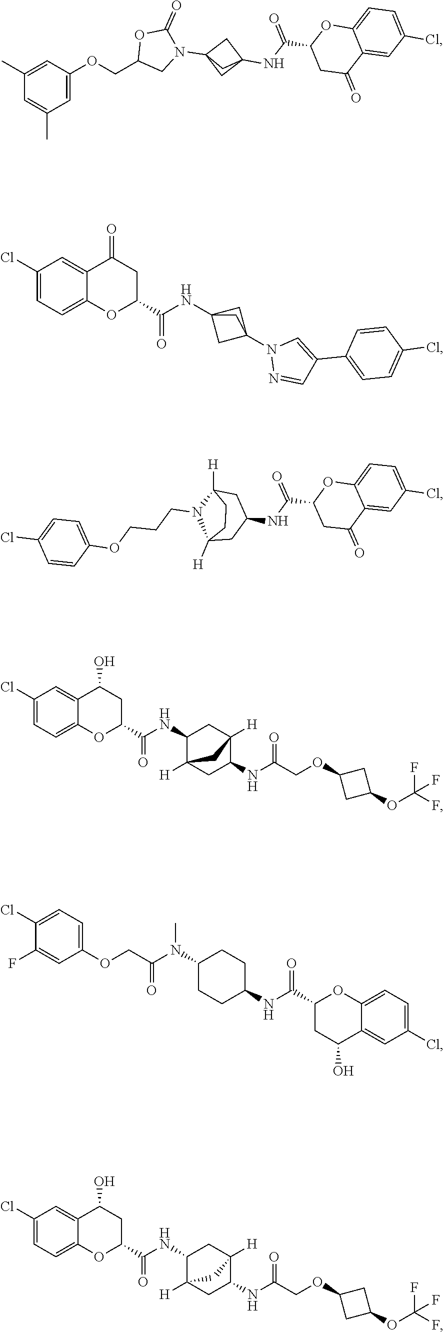 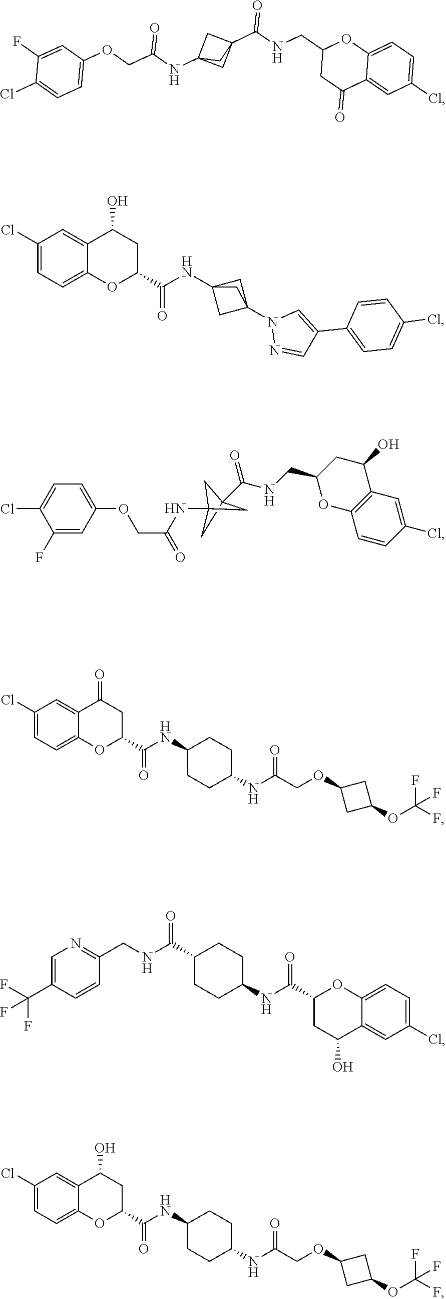 [SMILES: O=C(COc1ccc(Cl)c(F)c1)NC12CC(C(=O)NCC3CC(=O)c4cc(Cl)ccc4O3)(C1)C2 ; O=C(NC12CC(n3cc(-c4ccc(Cl)cc4)cn3)(C1)C2)[C@H]1C[C@@H](O)c2cc(Cl)ccc2O1 ; O=C(COc1ccc(Cl)c(F)c1)NC12CC(C(=O)NC[C@H]3C[C@@H](O)c4cc(Cl)ccc4O3)(C1)C2 ; O=C(CO[C@H]1C[C@@H](OC(F)(F)F)C1)N[C@H]1CC[C@H](NC(=O)[C@H]2CC(=O)c3cc(Cl)ccc3O2)CC1 ; O=C(N[C@H]1CC[C@H](C(=O)NCc2ccc(C(F)(F)F)cn2)CC1)[C@H]1C[C@@H](O)c2cc(Cl)ccc2O1 ; O=C(CO[C@H]1C[C@@H](OC(F)(F)F)C1)N[C@H]1CC[C@H](NC(=O)[C@H]2C[C@@H](O)c3cc(Cl)ccc3O2)CC1] 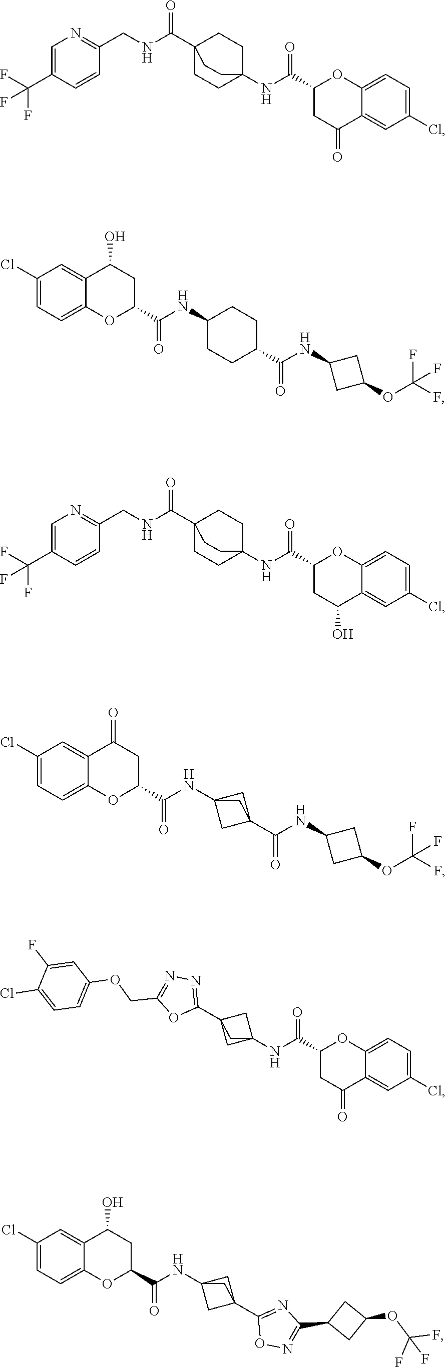 [SMILES: O=C1C[C@H](C(=O)NC23CCC(C(=O)NCc4ccc(C(F)(F)F)cn4)(CC2)CC3)Oc2ccc(Cl)cc21 ; O=C(N[C@H]1CC[C@H](C(=O)N[C@H]2C[C@@H](OC(F)(F)F)C2)CC1)[C@H]1C[C@@H](O)c2cc(Cl)ccc2O1 ; O=C(NC12CCC(C(=O)NCc3ccc(C(F)(F)F)cn3)(CC1)CC2)[C@H]1C[C@@H](O)c2cc(Cl)ccc2O1 ; O=C1C[C@H](C(=O)NC23CC(C(=O)N[C@H]4C[C@@H](OC(F)(F)F)C4)(C2)C3)Oc2ccc(Cl)cc21 ; O=C1C[C@H](C(=O)NC23CC(c4nnc(COc5ccc(Cl)c(F)c5)o4)(C2)C3)Oc2ccc(Cl)cc21 ; O=C(NC12CC(c3nc([C@H]4C[C@@H](OC(F)(F)F)C4)no3)(C1)C2)[C@@H]1C[C@@H](O)c2cc(Cl)ccc2O1] 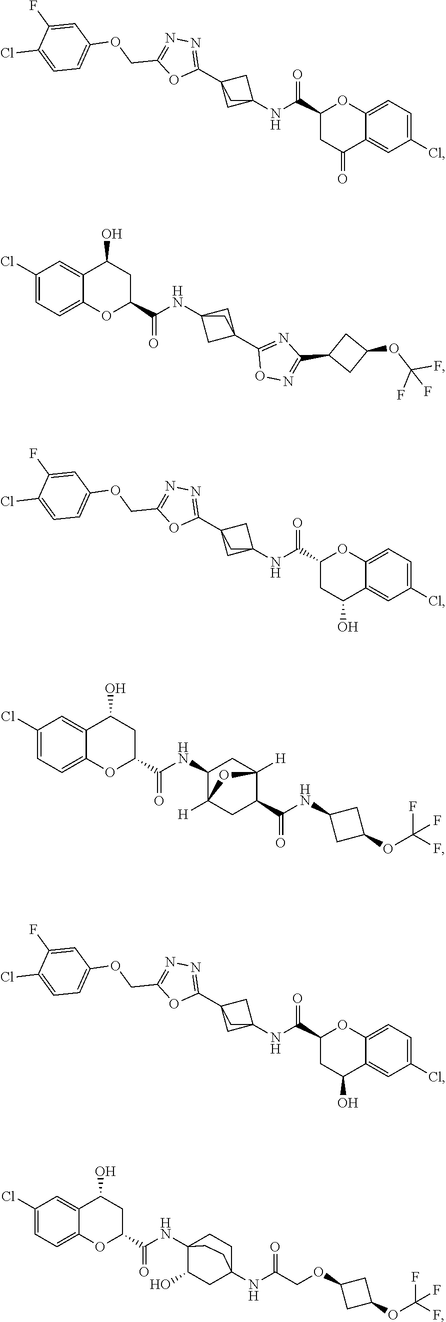 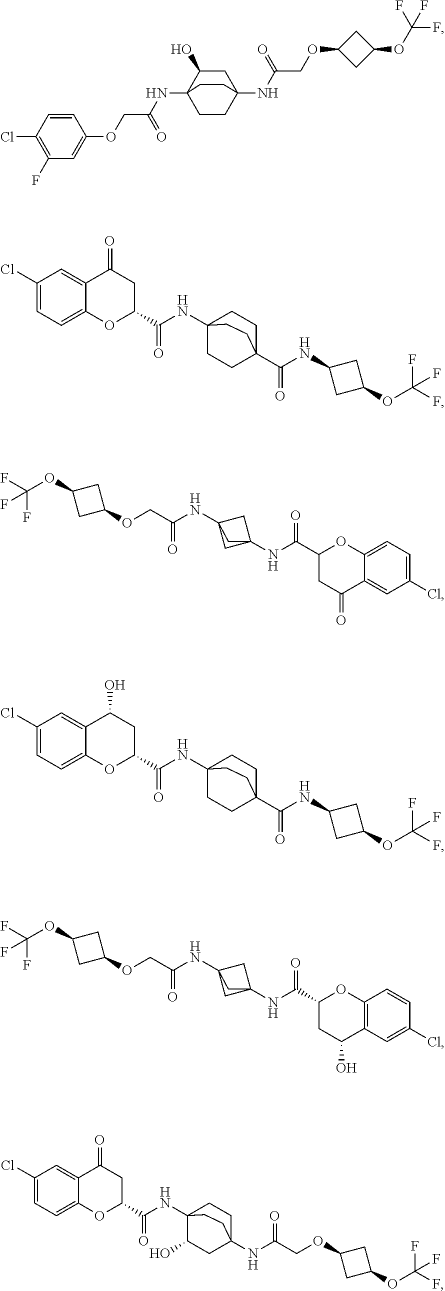 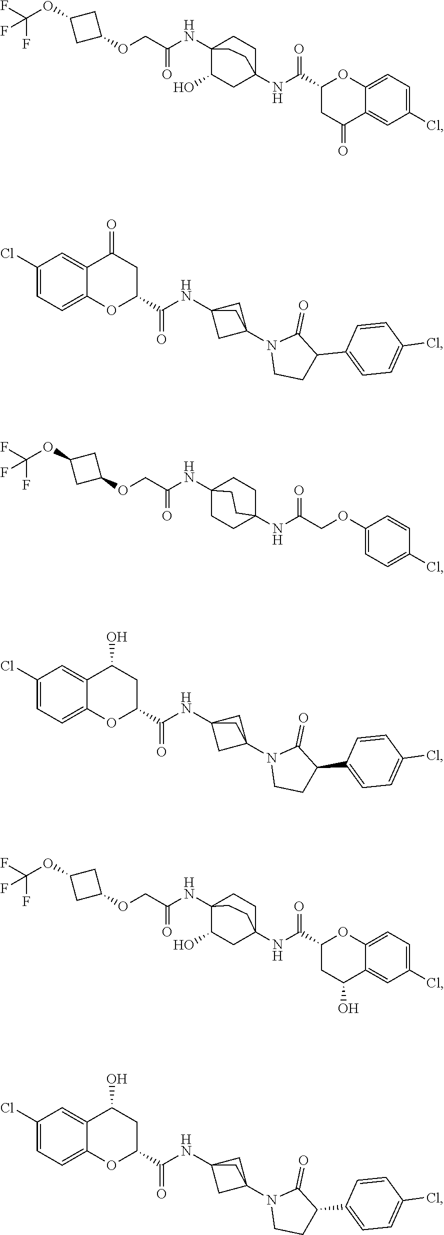 [SMILES: O=C(CO[C@H]1C[C@@H](OC(F)(F)F)C1)NC12CCC(NC(=O)[C@H]3CC(=O)c4cc(Cl)ccc4O3)(CC1)C[C@@H]2O ; O=C1C[C@H](C(=O)NC23CC(N4CCC(c5ccc(Cl)cc5)C4=O)(C2)C3)Oc2ccc(Cl)cc21 ; O=C(COc1ccc(Cl)cc1)NC12CCC(NC(=O)CO[C@H]3C[C@@H](OC(F)(F)F)C3)(CC1)CC2 ; O=C(NC12CC(N3CC[C@H](c4ccc(Cl)cc4)C3=O)(C1)C2)[C@H]1C[C@@H](O)c2cc(Cl)ccc2O1 ; O=C(CO[C@H]1C[C@@H](OC(F)(F)F)C1)NC12CCC(NC(=O)[C@H]3C[C@@H](O)c4cc(Cl)ccc4O3)(CC1)C[C@@H]2O ; O=C(NC12CC(N3CC[C@@H](c4ccc(Cl)cc4)C3=O)(C1)C2)[C@H]1C[C@@H](O)c2cc(Cl)ccc2O1] 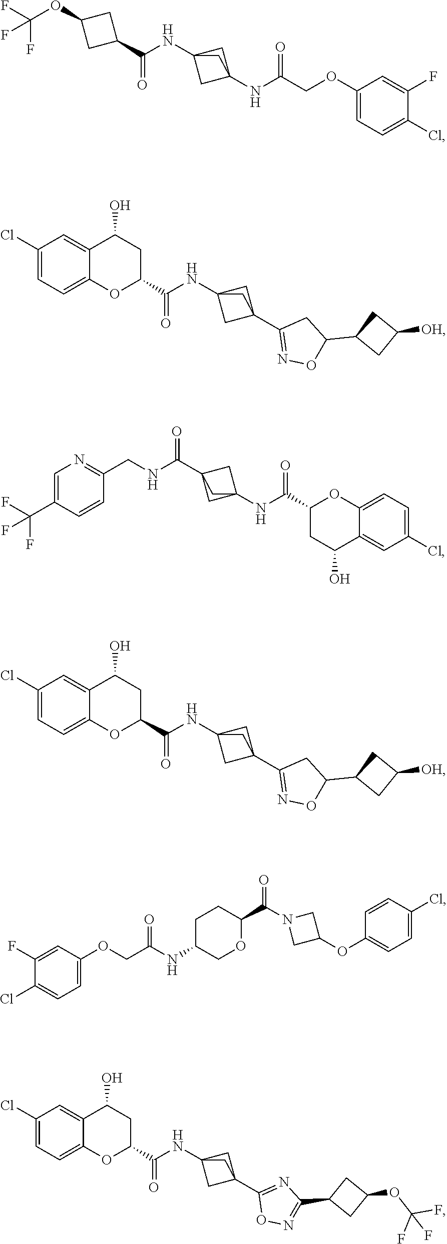 [SMILES: O=C(COc1ccc(Cl)c(F)c1)NC12CC(NC(=O)[C@H]3C[C@@H](OC(F)(F)F)C3)(C1)C2 ; O=C(NC12CC(C3=NOC([C@H]4C[C@@H](O)C4)C3)(C1)C2)[C@H]1C[C@@H](O)c2cc(Cl)ccc2O1 ; O=C(NC12CC(C(=O)NCc3ccc(C(F)(F)F)cn3)(C1)C2)[C@H]1C[C@@H](O)c2cc(Cl)ccc2O1 ; O=C(NC12CC(C3=NOC([C@H]4C[C@@H](O)C4)C3)(C1)C2)[C@@H]1C[C@@H](O)c2cc(Cl)ccc2O1 ; O=C(COc1ccc(Cl)c(F)c1)N[C@@H]1CC[C@@H](C(=O)N2CC(Oc3ccc(Cl)cc3)C2)OC1 ; O=C(NC12CC(c3nc([C@H]4C[C@@H](OC(F)(F)F)C4)no3)(C1)C2)[C@H]1C[C@@H](O)c2cc(Cl)ccc2O1] 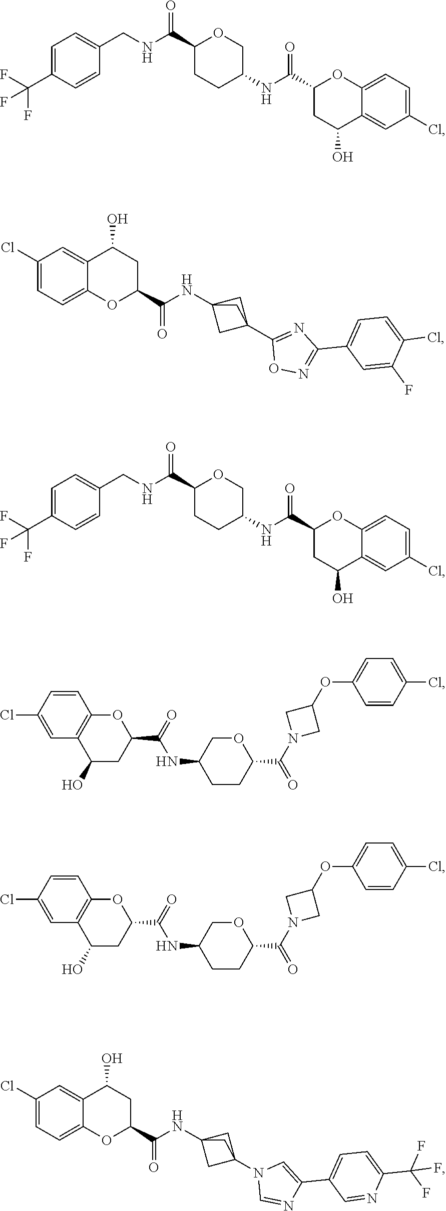 [SMILES: O=C(NCc1ccc(C(F)(F)F)cc1)[C@@H]1CC[C@@H](NC(=O)[C@H]2C[C@@H](O)c3cc(Cl)ccc3O2)CO1 ; O=C(NC12CC(c3nc(-c4ccc(Cl)c(F)c4)no3)(C1)C2)[C@@H]1C[C@@H](O)c2cc(Cl)ccc2O1 ; O=C(NCc1ccc(C(F)(F)F)cc1)[C@@H]1CC[C@@H](NC(=O)[C@@H]2C[C@H](O)c3cc(Cl)ccc3O2)CO1 ; O=C(N[C@@H]1CC[C@@H](C(=O)N2CC(Oc3ccc(Cl)cc3)C2)OC1)[C@H]1C[C@@H](O)c2cc(Cl)ccc2O1 ; O=C(N[C@@H]1CC[C@@H](C(=O)N2CC(Oc3ccc(Cl)cc3)C2)OC1)[C@@H]1C[C@H](O)c2cc(Cl)ccc2O1 ; O=C(NC12CC(n3cnc(-c4ccc(C(F)(F)F)nc4)c3)(C1)C2)[C@@H]1C[C@@H](O)c2cc(Cl)ccc2O1] 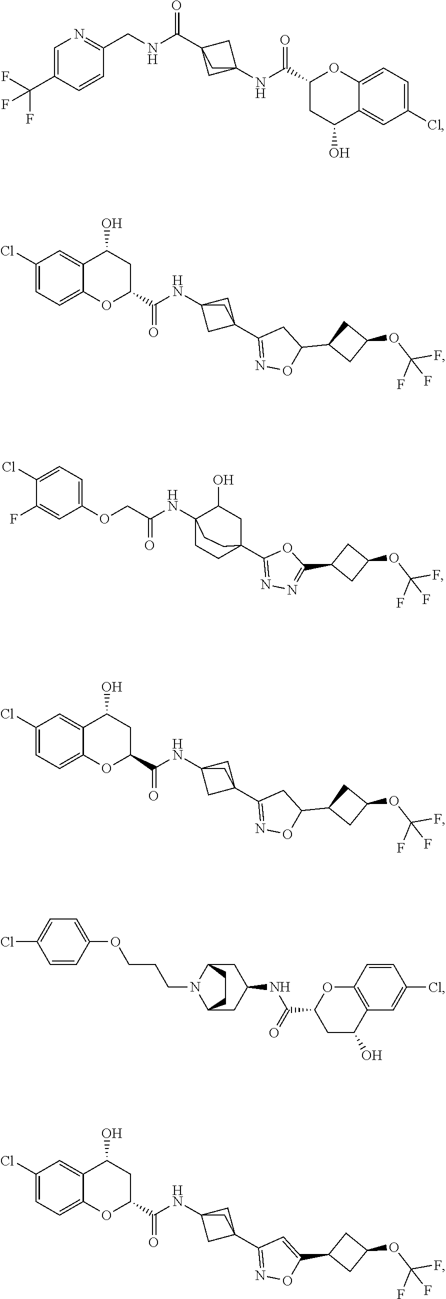 [SMILES: O=C(NC12CC(C(=O)NCc3ccc(C(F)(F)F)cn3)(C1)C2)[C@H]1C[C@@H](O)c2cc(Cl)ccc2O1 ; O=C(NC12CC(C3=NOC([C@H]4C[C@@H](OC(F)(F)F)C4)C3)(C1)C2)[C@H]1C[C@@H](O)c2cc(Cl)ccc2O1 ; O=C(COc1ccc(Cl)c(F)c1)NC12CCC(c3nnc([C@H]4C[C@@H](OC(F)(F)F)C4)o3)(CC1)CC2O ; O=C(NC12CC(C3=NOC([C@H]4C[C@@H](OC(F)(F)F)C4)C3)(C1)C2)[C@@H]1C[C@@H](O)c2cc(Cl)ccc2O1 ; O=C(N[C@H]1C[C@H]2CC[C@@H](C1)N2CCCOc1ccc(Cl)cc1)[C@H]1C[C@@H](O)c2cc(Cl)ccc2O1 ; O=C(NC12CC(c3cc([C@H]4C[C@@H](OC(F)(F)F)C4)on3)(C1)C2)[C@H]1C[C@@H](O)c2cc(Cl)ccc2O1] 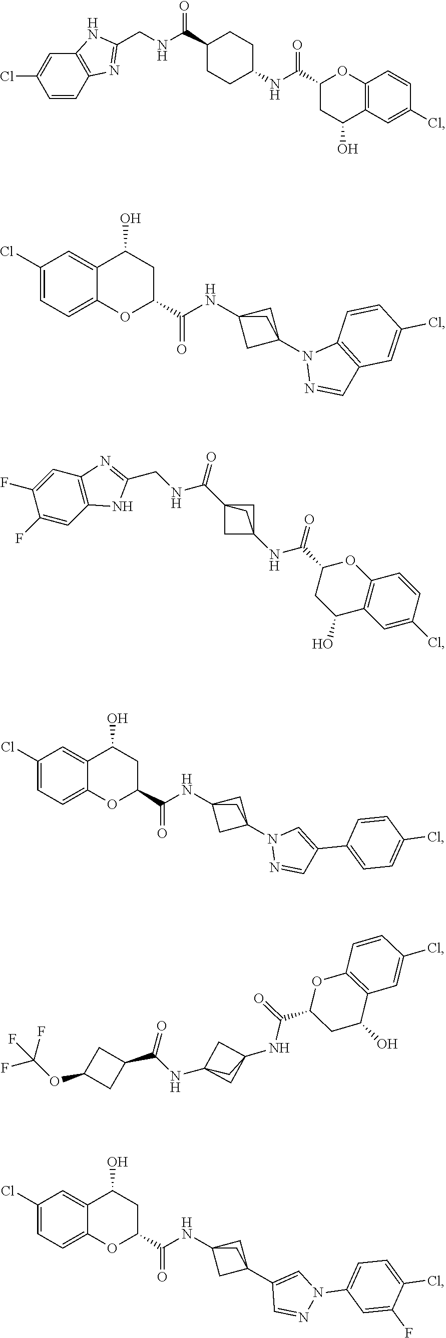 [SMILES: O=C(N[C@H]1CC[C@H](C(=O)NCc2nc3ccc(Cl)cc3[nH]2)CC1)[C@H]1C[C@@H](O)c2cc(Cl)ccc2O1 ; O=C(NC12CC(n3ncc4cc(Cl)ccc43)(C1)C2)[C@H]1C[C@@H](O)c2cc(Cl)ccc2O1 ; O=C(NC12CC(C(=O)NCc3nc4cc(F)c(F)cc4[nH]3)(C1)C2)[C@H]1C[C@@H](O)c2cc(Cl)ccc2O1 ; O=C(NC12CC(n3cc(-c4ccc(Cl)cc4)cn3)(C1)C2)[C@@H]1C[C@@H](O)c2cc(Cl)ccc2O1 ; O=C(NC12CC(NC(=O)[C@H]3C[C@@H](OC(F)(F)F)C3)(C1)C2)[C@H]1C[C@@H](O)c2cc(Cl)ccc2O1 ; O=C(NC12CC(c3cnn(-c4ccc(Cl)c(F)c4)c3)(C1)C2)[C@H]1C[C@@H](O)c2cc(Cl)ccc2O1] 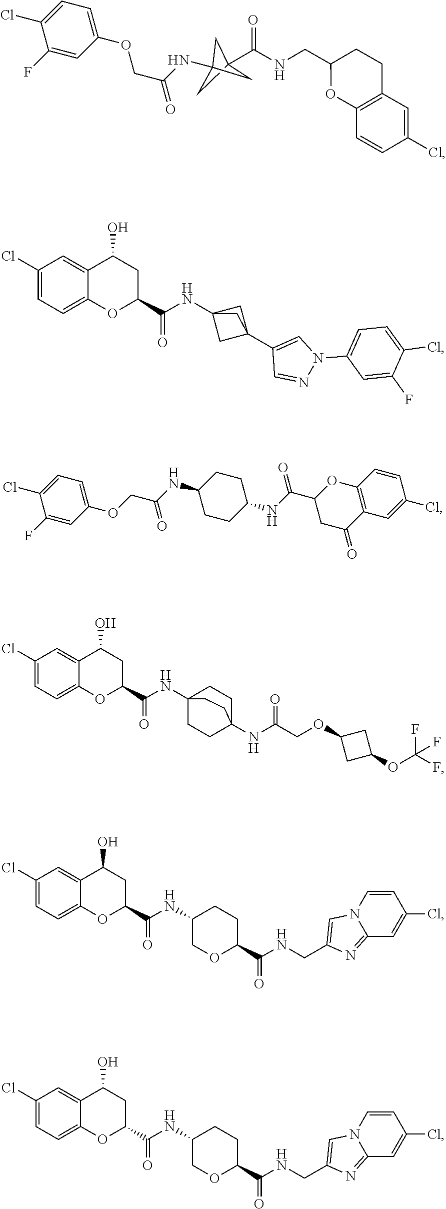 [SMILES: O=C(COc1ccc(Cl)c(F)c1)NC12CC(C(=O)NCC3CCc4cc(Cl)ccc4O3)(C1)C2 ; O=C(NC12CC(c3cnn(-c4ccc(Cl)c(F)c4)c3)(C1)C2)[C@@H]1C[C@@H](O)c2cc(Cl)ccc2O1 ; O=C(COc1ccc(Cl)c(F)c1)N[C@H]1CC[C@H](NC(=O)C2CC(=O)c3cc(Cl)ccc3O2)CC1 ; O=C(CO[C@H]1C[C@@H](OC(F)(F)F)C1)NC12CCC(NC(=O)[C@@H]3C[C@@H](O)c4cc(Cl)ccc4O3)(CC1)CC2 ; O=C(NCc1cn2ccc(Cl)cc2n1)[C@@H]1CC[C@@H](NC(=O)[C@@H]2C[C@H](O)c3cc(Cl)ccc3O2)CO1 ; O=C(NCc1cn2ccc(Cl)cc2n1)[C@@H]1CC[C@@H](NC(=O)[C@H]2C[C@@H](O)c3cc(Cl)ccc3O2)CO1] 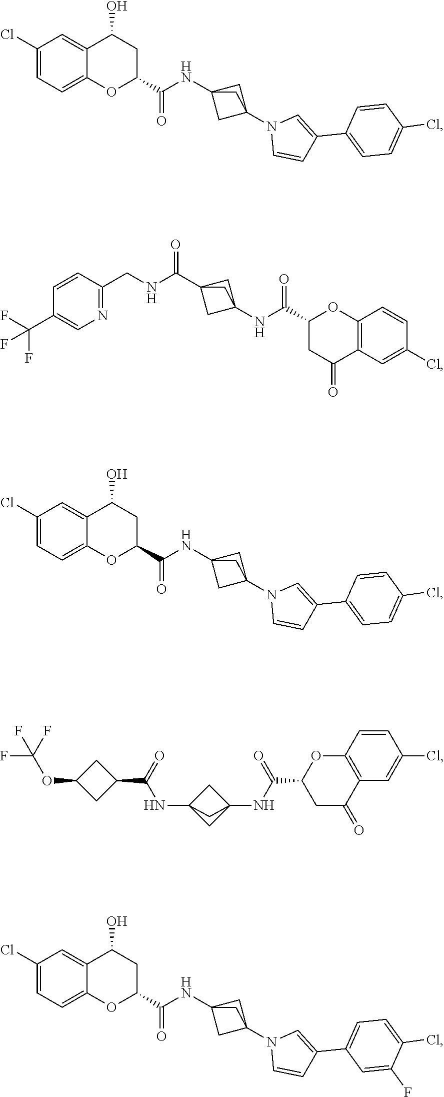 [SMILES: O=C(NC12CC(n3ccc(-c4ccc(Cl)cc4)c3)(C1)C2)[C@H]1C[C@@H](O)c2cc(Cl)ccc2O1 ; O=C1C[C@H](C(=O)NC23CC(C(=O)NCc4ccc(C(F)(F)F)cn4)(C2)C3)Oc2ccc(Cl)cc21 ; O=C(NC12CC(n3ccc(-c4ccc(Cl)cc4)c3)(C1)C2)[C@@H]1C[C@@H](O)c2cc(Cl)ccc2O1 ; O=C1C[C@H](C(=O)NC23CC(NC(=O)[C@H]4C[C@@H](OC(F)(F)F)C4)(C2)C3)Oc2ccc(Cl)cc21 ; O=C(NC12CC(n3ccc(-c4ccc(Cl)c(F)c4)c3)(C1)C2)[C@H]1C[C@@H](O)c2cc(Cl)ccc2O1] 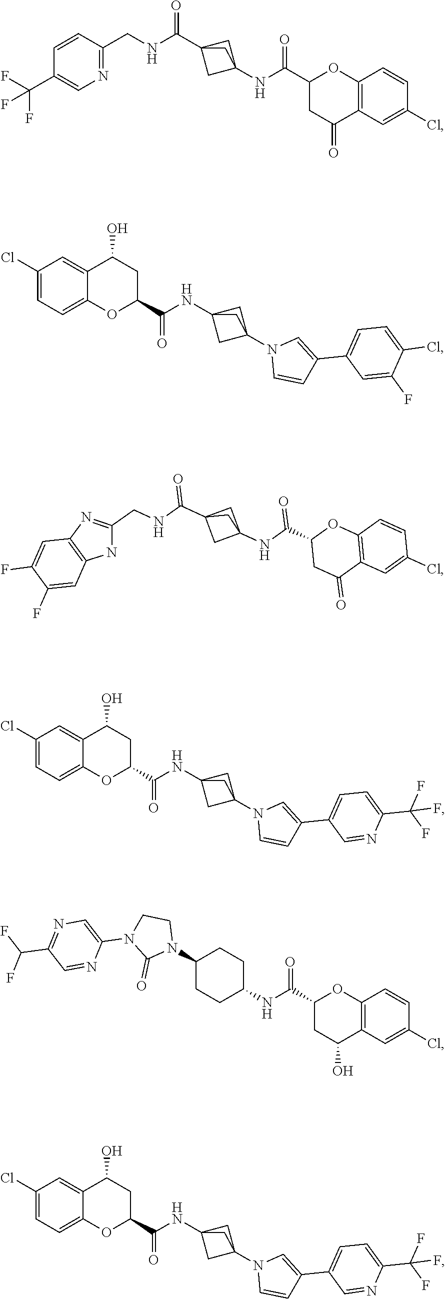 [SMILES: O=C1CC(C(=O)NC23CC(C(=O)NCc4ccc(C(F)(F)F)cn4)(C2)C3)Oc2ccc(Cl)cc21 ; O=C(NC12CC(n3ccc(-c4ccc(Cl)c(F)c4)c3)(C1)C2)[C@@H]1C[C@@H](O)c2cc(Cl)ccc2O1 ; O=C1C[C@H](C(=O)NC23CC(C(=O)NCc4nc5cc(F)c(F)cc5[nH]4)(C2)C3)Oc2ccc(Cl)cc21 ; O=C(NC12CC(n3ccc(-c4ccc(C(F)(F)F)nc4)c3)(C1)C2)[C@H]1C[C@@H](O)c2cc(Cl)ccc2O1 ; O=C(N[C@H]1CC[C@H](N2CCN(c3cnc(C(F)F)cn3)C2=O)CC1)[C@H]1C[C@@H](O)c2cc(Cl)ccc2O1 ; O=C(NC12CC(n3ccc(-c4ccc(C(F)(F)F)nc4)c3)(C1)C2)[C@@H]1C[C@@H](O)c2cc(Cl)ccc2O1] 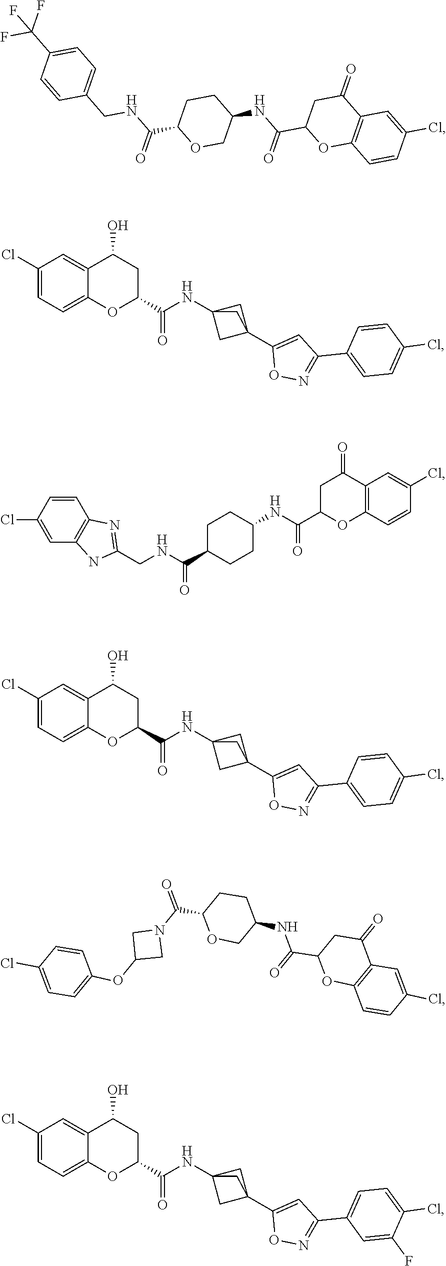 [SMILES: O=C1CC(C(=O)N[C@@H]2CC[C@@H](C(=O)NCc3ccc(C(F)(F)F)cc3)OC2)Oc2ccc(Cl)cc21 ; O=C(NC12CC(c3cc(-c4ccc(Cl)cc4)no3)(C1)C2)[C@H]1C[C@@H](O)c2cc(Cl)ccc2O1 ; O=C1CC(C(=O)N[C@H]2CC[C@H](C(=O)NCc3nc4ccc(Cl)cc4[nH]3)CC2)Oc2ccc(Cl)cc21 ; O=C(NC12CC(c3cc(-c4ccc(Cl)cc4)no3)(C1)C2)[C@@H]1C[C@@H](O)c2cc(Cl)ccc2O1 ; O=C1CC(C(=O)N[C@@H]2CC[C@@H](C(=O)N3CC(Oc4ccc(Cl)cc4)C3)OC2)Oc2ccc(Cl)cc21 ; O=C(NC12CC(c3cc(-c4ccc(Cl)c(F)c4)no3)(C1)C2)[C@H]1C[C@@H](O)c2cc(Cl)ccc2O1] 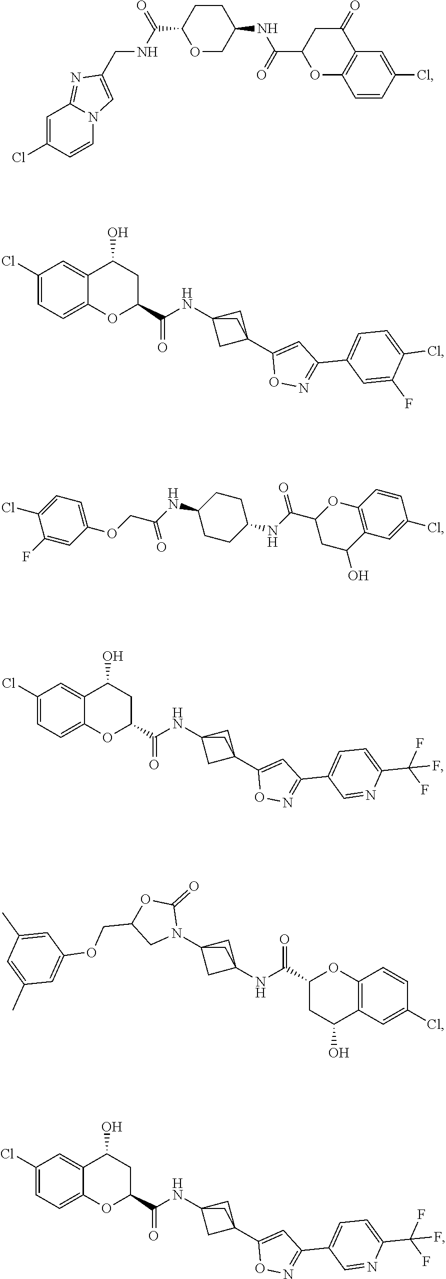 [SMILES: O=C1CC(C(=O)N[C@@H]2CC[C@@H](C(=O)NCc3cn4ccc(Cl)cc4n3)OC2)Oc2ccc(Cl)cc21 ; O=C(NC12CC(c3cc(-c4ccc(Cl)c(F)c4)no3)(C1)C2)[C@@H]1C[C@@H](O)c2cc(Cl)ccc2O1 ; O=C(COc1ccc(Cl)c(F)c1)N[C@H]1CC[C@H](NC(=O)C2CC(O)c3cc(Cl)ccc3O2)CC1 ; O=C(NC12CC(c3cc(-c4ccc(C(F)(F)F)nc4)no3)(C1)C2)[C@H]1C[C@@H](O)c2cc(Cl)ccc2O1 ; Cc1cc(C)cc(OCC2CN(C34CC(NC(=O)[C@H]5C[C@@H](O)c6cc(Cl)ccc6O5)(C3)C4)C(=O)O2)c1 ; O=C(NC12CC(c3cc(-c4ccc(C(F)(F)F)nc4)no3)(C1)C2)[C@@H]1C[C@@H](O)c2cc(Cl)ccc2O1] 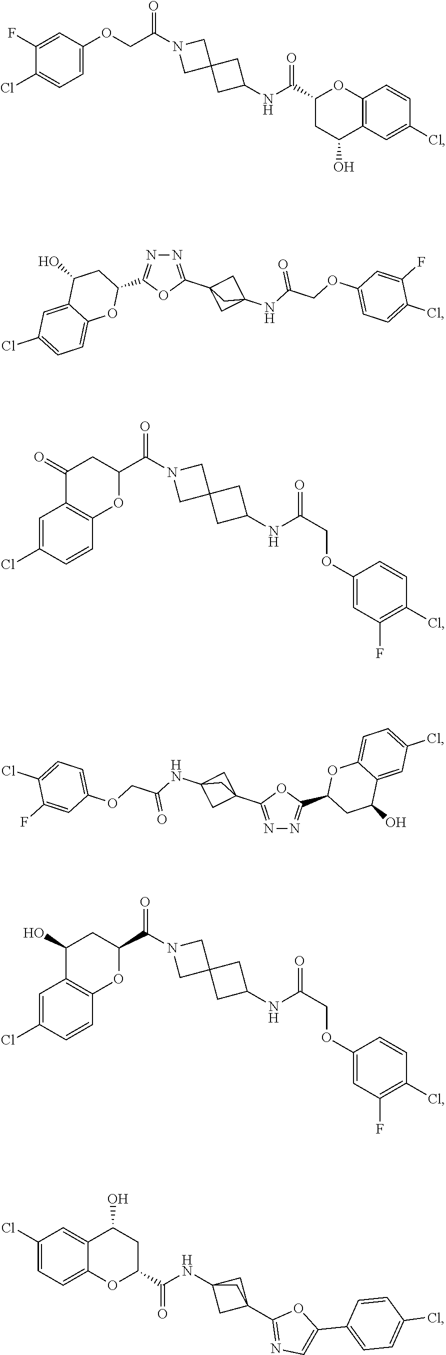 [SMILES: O=C(NC1CC2(C1)CN(C(=O)COc1ccc(Cl)c(F)c1)C2)[C@H]1C[C@@H](O)c2cc(Cl)ccc2O1 ; O=C(COc1ccc(Cl)c(F)c1)NC12CC(c3nnc([C@H]4C[C@@H](O)c5cc(Cl)ccc5O4)o3)(C1)C2 ; O=C(COc1ccc(Cl)c(F)c1)NC1CC2(C1)CN(C(=O)C1CC(=O)c3cc(Cl)ccc3O1)C2 ; O=C(COc1ccc(Cl)c(F)c1)NC12CC(c3nnc([C@@H]4C[C@H](O)c5cc(Cl)ccc5O4)o3)(C1)C2 ; O=C(COc1ccc(Cl)c(F)c1)NC1CC2(C1)CN(C(=O)[C@@H]1C[C@H](O)c3cc(Cl)ccc3O1)C2 ; O=C(NC12CC(c3ncc(-c4ccc(Cl)cc4)o3)(C1)C2)[C@H]1C[C@@H](O)c2cc(Cl)ccc2O1] 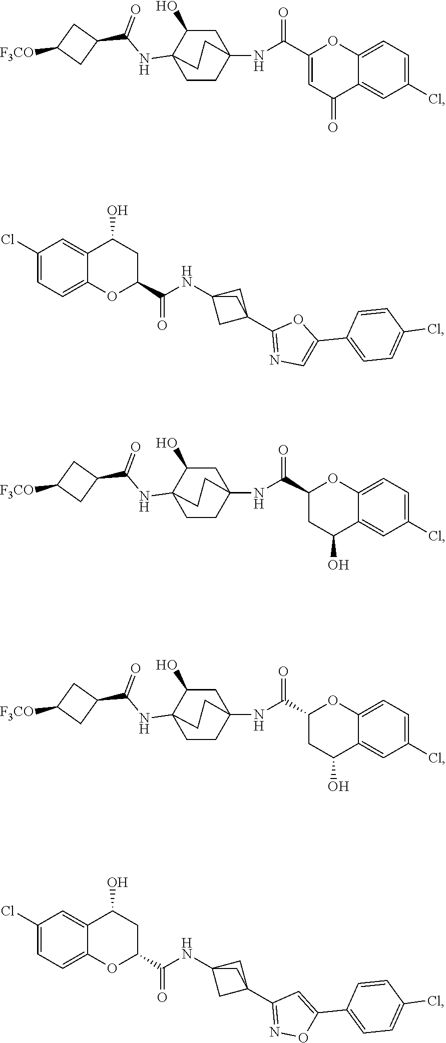 [SMILES: O=C(NC12CCC(NC(=O)[C@H]3C[C@@H](OC(F)(F)F)C3)(CC1)[C@@H](O)C2)c1cc(=O)c2cc(Cl)ccc2o1 ; O=C(NC12CC(c3ncc(-c4ccc(Cl)cc4)o3)(C1)C2)[C@@H]1C[C@@H](O)c2cc(Cl)ccc2O1 ; O=C(NC12CCC(NC(=O)[C@H]3C[C@@H](OC(F)(F)F)C3)(CC1)[C@@H](O)C2)[C@@H]1C[C@H](O)c2cc(Cl)ccc2O1 ; O=C(NC12CCC(NC(=O)[C@H]3C[C@@H](OC(F)(F)F)C3)(CC1)[C@@H](O)C2)[C@H]1C[C@@H](O)c2cc(Cl)ccc2O1 ; O=C(NC12CC(c3cc(-c4ccc(Cl)cc4)on3)(C1)C2)[C@H]1C[C@@H](O)c2cc(Cl)ccc2O1] 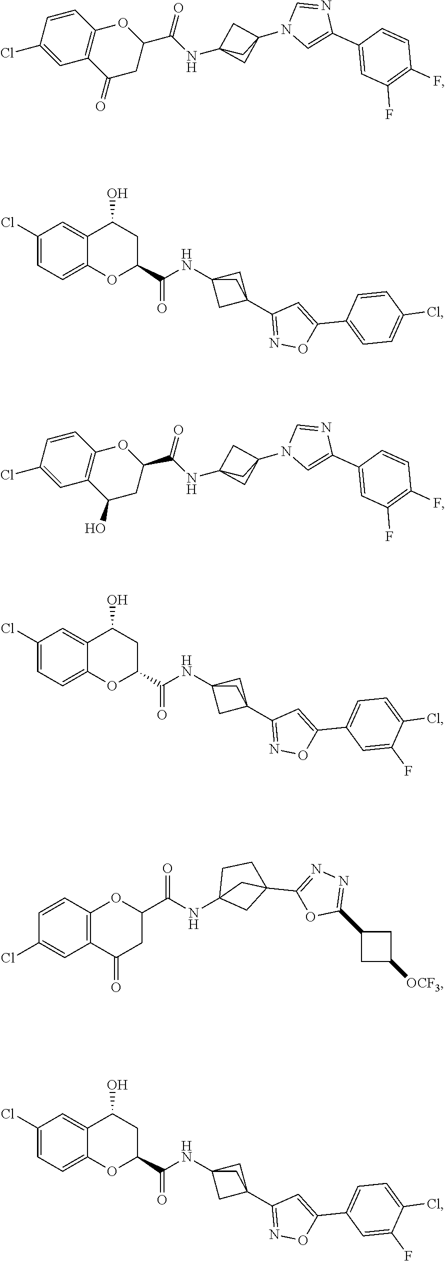 [SMILES: O=C1CC(C(=O)NC23CC(n4cnc(-c5ccc(F)c(F)c5)c4)(C2)C3)Oc2ccc(Cl)cc21 ; O=C(NC12CC(c3cc(-c4ccc(Cl)cc4)on3)(C1)C2)[C@@H]1C[C@@H](O)c2cc(Cl)ccc2O1 ; O=C(NC12CC(n3cnc(-c4ccc(F)c(F)c4)c3)(C1)C2)[C@H]1C[C@@H](O)c2cc(Cl)ccc2O1 ; O=C(NC12CC(c3cc(-c4ccc(Cl)c(F)c4)on3)(C1)C2)[C@H]1C[C@@H](O)c2cc(Cl)ccc2O1 ; O=C1CC(C(=O)NC23CCC(c4nnc([C@H]5C[C@@H](OC(F)(F)F)C5)o4)(C2)C3)Oc2ccc(Cl)cc21 ; O=C(NC12CC(c3cc(-c4ccc(Cl)c(F)c4)on3)(C1)C2)[C@@H]1C[C@@H](O)c2cc(Cl)ccc2O1] 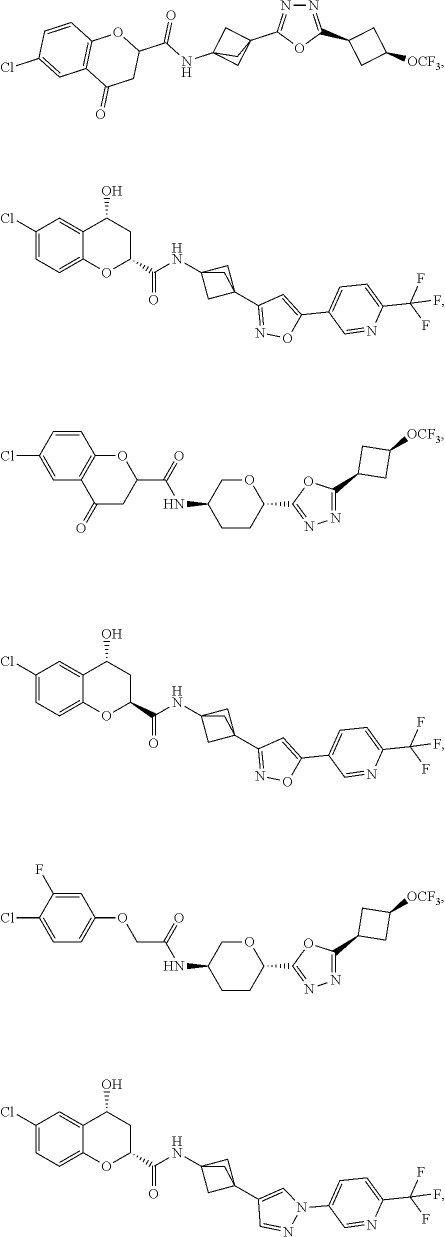 [SMILES: O=C1CC(C(=O)NC23CC(c4nnc([C@H]5C[C@@H](OC(F)(F)F)C5)o4)(C2)C3)Oc2ccc(Cl)cc21 ; O=C(NC12CC(c3cc(-c4ccc(C(F)(F)F)nc4)on3)(C1)C2)[C@H]1C[C@@H](O)c2cc(Cl)ccc2O1 ; O=C1CC(C(=O)N[C@@H]2CC[C@@H](c3nnc([C@H]4C[C@@H](OC(F)(F)F)C4)o3)OC2)Oc2ccc(Cl)cc21 ; O=C(NC12CC(c3cc(-c4ccc(C(F)(F)F)nc4)on3)(C1)C2)[C@@H]1C[C@@H](O)c2cc(Cl)ccc2O1 ; O=C(COc1ccc(Cl)c(F)c1)N[C@@H]1CC[C@@H](c2nnc([C@H]3C[C@@H](OC(F)(F)F)C3)o2)OC1 ; O=C(NC12CC(c3cnn(-c4ccc(C(F)(F)F)nc4)c3)(C1)C2)[C@H]1C[C@@H](O)c2cc(Cl)ccc2O1] 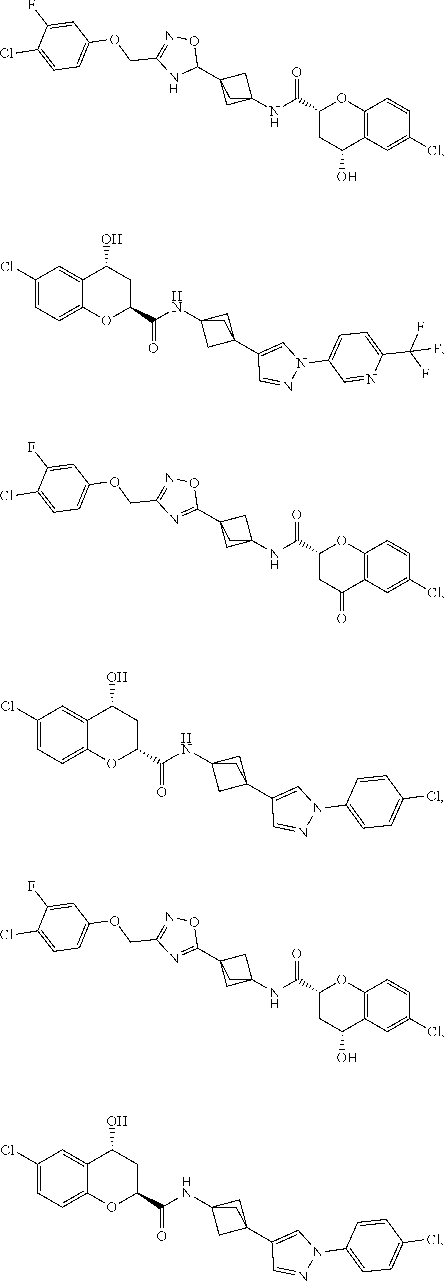 [SMILES: O=C(NC12CC(C3NC(COc4ccc(Cl)c(F)c4)=NO3)(C1)C2)[C@H]1C[C@@H](O)c2cc(Cl)ccc2O1 ; O=C(NC12CC(c3cnn(-c4ccc(C(F)(F)F)nc4)c3)(C1)C2)[C@@H]1C[C@@H](O)c2cc(Cl)ccc2O1 ; O=C1C[C@H](C(=O)NC23CC(c4nc(COc5ccc(Cl)c(F)c5)no4)(C2)C3)Oc2ccc(Cl)cc21 ; O=C(NC12CC(c3cnn(-c4ccc(Cl)cc4)c3)(C1)C2)[C@H]1C[C@@H](O)c2cc(Cl)ccc2O1 ; O=C(NC12CC(c3nc(COc4ccc(Cl)c(F)c4)no3)(C1)C2)[C@H]1C[C@@H](O)c2cc(Cl)ccc2O1 ; O=C(NC12CC(c3cnn(-c4ccc(Cl)cc4)c3)(C1)C2)[C@@H]1C[C@@H](O)c2cc(Cl)ccc2O1] 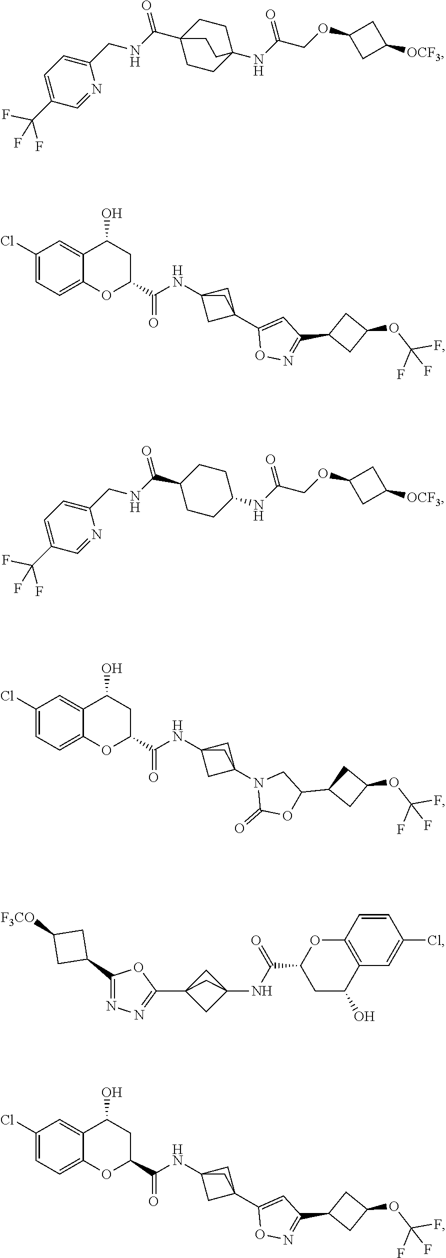 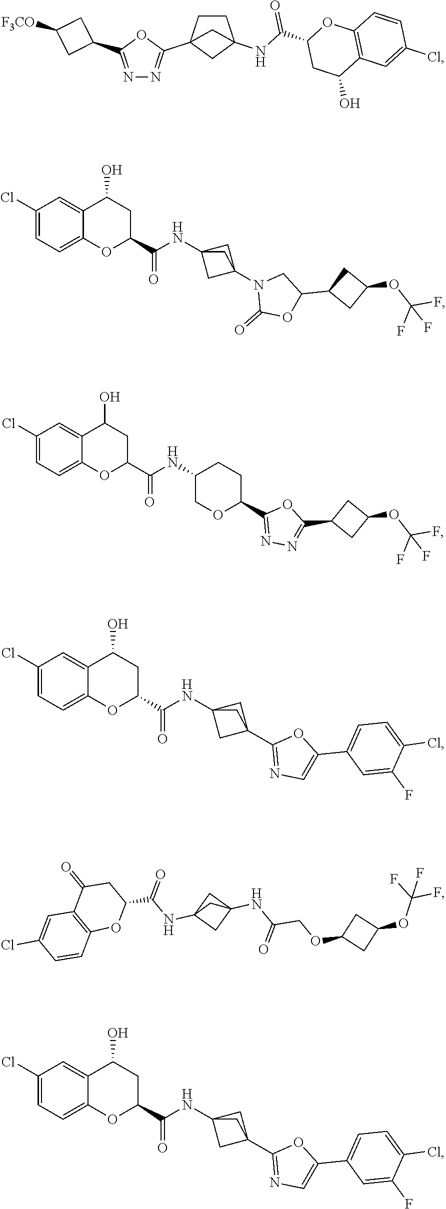 [SMILES: O=C(NC12CCC(c3nnc([C@H]4C[C@@H](OC(F)(F)F)C4)o3)(C1)C2)[C@H]1C[C@@H](O)c2cc(Cl)ccc2O1 ; O=C(NC12CC(N3CC([C@H]4C[C@@H](OC(F)(F)F)C4)OC3=O)(C1)C2)[C@@H]1C[C@@H](O)c2cc(Cl)ccc2O1 ; O=C(N[C@@H]1CC[C@@H](c2nnc([C@H]3C[C@@H](OC(F)(F)F)C3)o2)OC1)C1CC(O)c2cc(Cl)ccc2O1 ; O=C(NC12CC(c3ncc(-c4ccc(Cl)c(F)c4)o3)(C1)C2)[C@H]1C[C@@H](O)c2cc(Cl)ccc2O1 ; O=C(CO[C@H]1C[C@@H](OC(F)(F)F)C1)NC12CC(NC(=O)[C@H]3CC(=O)c4cc(Cl)ccc4O3)(C1)C2 ; O=C(NC12CC(c3ncc(-c4ccc(Cl)c(F)c4)o3)(C1)C2)[C@@H]1C[C@@H](O)c2cc(Cl)ccc2O1] 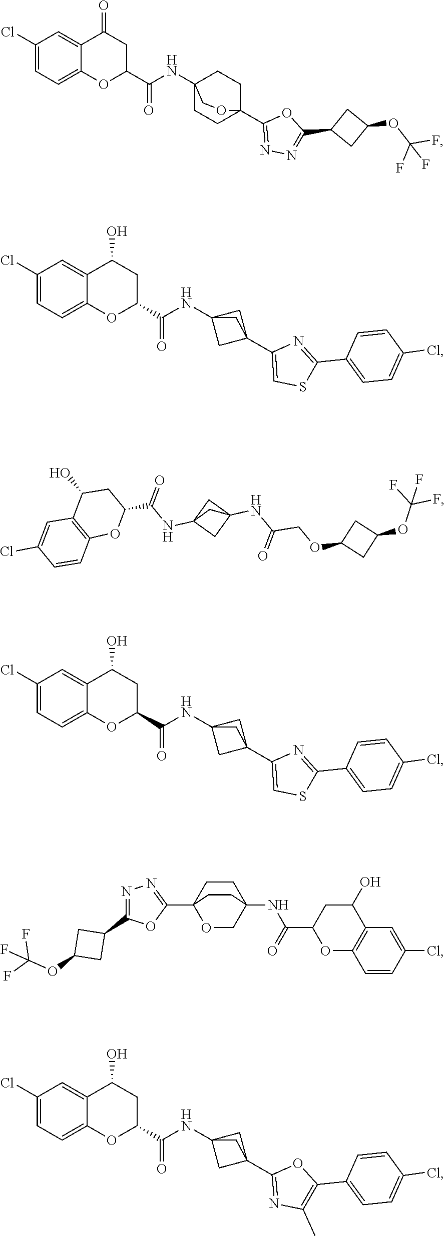 [SMILES: O=C1CC(C(=O)NC23CCC(c4nnc([C@H]5C[C@@H](OC(F)(F)F)C5)o4)(CC2)OC3)Oc2ccc(Cl)cc21 ; O=C(NC12CC(c3csc(-c4ccc(Cl)cc4)n3)(C1)C2)[C@H]1C[C@@H](O)c2cc(Cl)ccc2O1 ; O=C(CO[C@H]1C[C@@H](OC(F)(F)F)C1)NC12CC(NC(=O)[C@H]3C[C@@H](O)c4cc(Cl)ccc4O3)(C1)C2 ; O=C(NC12CC(c3csc(-c4ccc(Cl)cc4)n3)(C1)C2)[C@@H]1C[C@@H](O)c2cc(Cl)ccc2O1 ; O=C(NC12CCC(c3nnc([C@H]4C[C@@H](OC(F)(F)F)C4)o3)(CC1)OC2)C1CC(O)c2cc(Cl)ccc2O1 ; Cc1nc(C23CC(NC(=O)[C@H]4C[C@@H](O)c5cc(Cl)ccc5O4)(C2)C3)oc1-c1ccc(Cl)cc1] 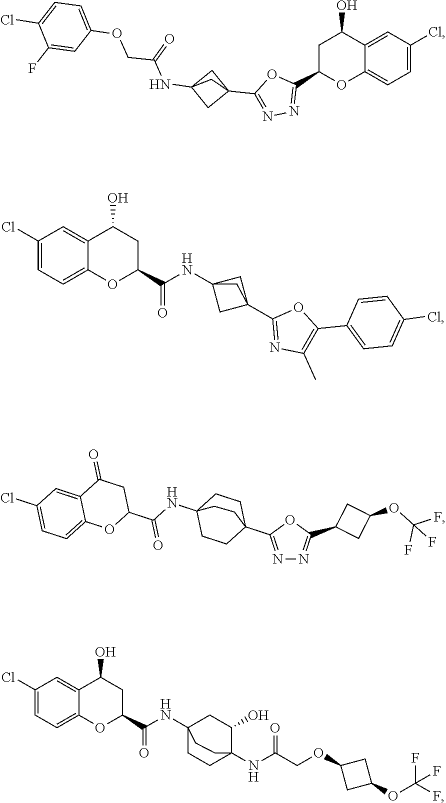 [SMILES: O=C(COc1ccc(Cl)c(F)c1)NC12CC(c3nnc([C@H]4C[C@@H](O)c5cc(Cl)ccc5O4)o3)(C1)C2 ; Cc1nc(C23CC(NC(=O)[C@@H]4C[C@@H](O)c5cc(Cl)ccc5O4)(C2)C3)oc1-c1ccc(Cl)cc1 ; O=C1CC(C(=O)NC23CCC(c4nnc([C@H]5C[C@@H](OC(F)(F)F)C5)o4)(CC2)CC3)Oc2ccc(Cl)cc21 ; O=C(CO[C@H]1C[C@@H](OC(F)(F)F)C1)NC12CCC(NC(=O)[C@@H]3C[C@H](O)c4cc(Cl)ccc4O3)(CC1)C[C@@H]2O] 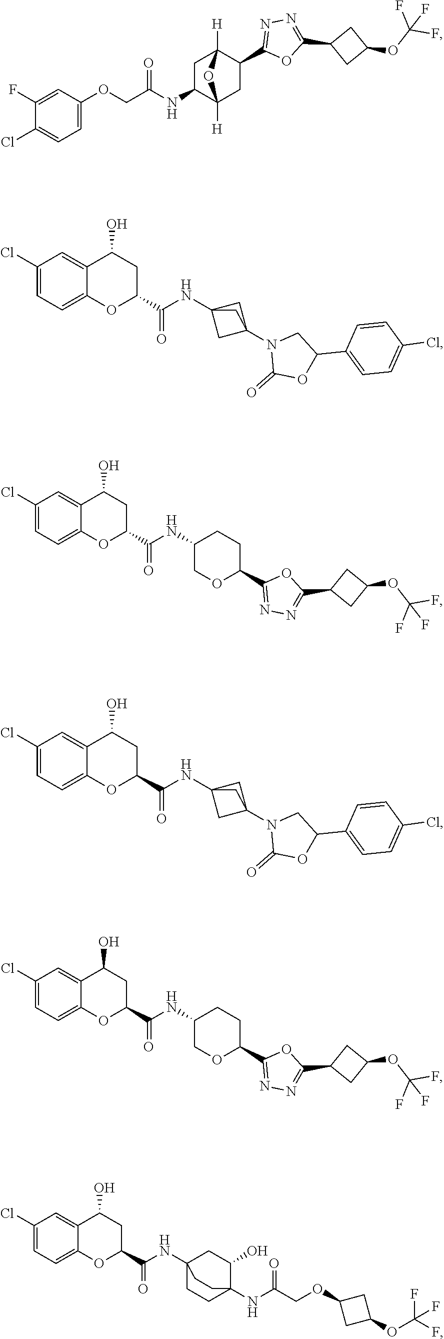 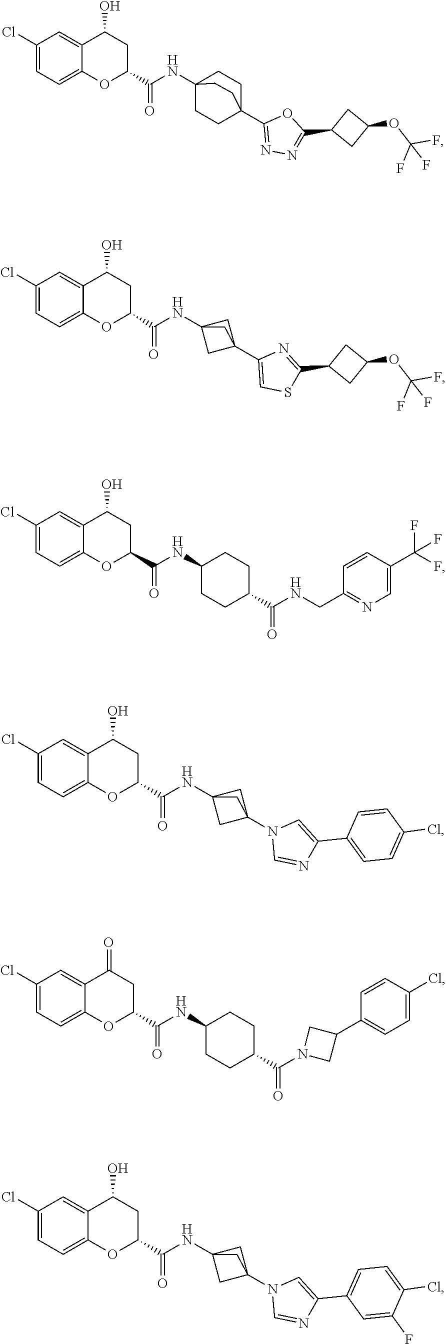 [SMILES: O=C(NC12CCC(c3nnc([C@H]4C[C@@H](OC(F)(F)F)C4)o3)(CC1)CC2)[C@H]1C[C@@H](O)c2cc(Cl)ccc2O1 ; O=C(NC12CC(c3csc([C@H]4C[C@@H](OC(F)(F)F)C4)n3)(C1)C2)[C@H]1C[C@@H](O)c2cc(Cl)ccc2O1 ; O=C(N[C@H]1CC[C@H](C(=O)NCc2ccc(C(F)(F)F)cn2)CC1)[C@@H]1C[C@@H](O)c2cc(Cl)ccc2O1 ; O=C(NC12CC(n3cnc(-c4ccc(Cl)cc4)c3)(C1)C2)[C@H]1C[C@@H](O)c2cc(Cl)ccc2O1 ; O=C1C[C@H](C(=O)N[C@H]2CC[C@H](C(=O)N3CC(c4ccc(Cl)cc4)C3)CC2)Oc2ccc(Cl)cc21 ; O=C(NC12CC(n3cnc(-c4ccc(Cl)c(F)c4)c3)(C1)C2)[C@H]1C[C@@H](O)c2cc(Cl)ccc2O1] 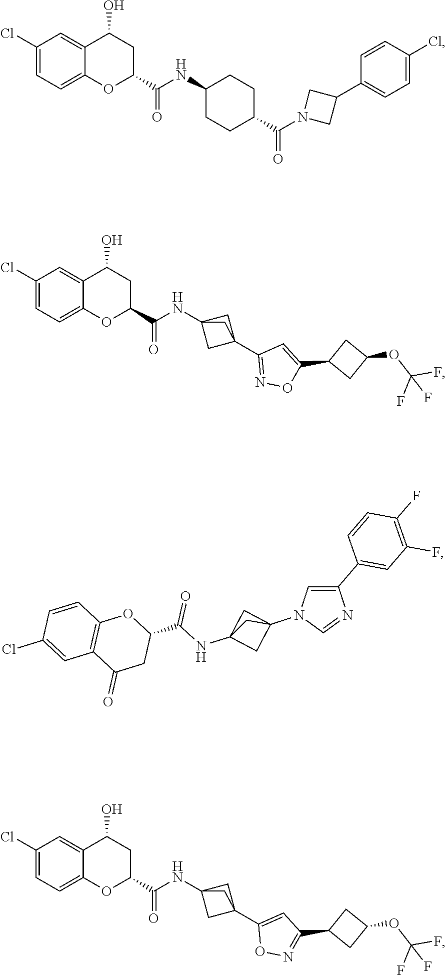 [SMILES: O=C(N[C@H]1CC[C@H](C(=O)N2CC(c3ccc(Cl)cc3)C2)CC1)[C@H]1C[C@@H](O)c2cc(Cl)ccc2O1 ; O=C(NC12CC(c3cc([C@H]4C[C@@H](OC(F)(F)F)C4)on3)(C1)C2)[C@@H]1C[C@@H](O)c2cc(Cl)ccc2O1 ; O=C1C[C@@H](C(=O)NC23CC(n4cnc(-c5ccc(F)c(F)c5)c4)(C2)C3)Oc2ccc(Cl)cc21 ; O=C(NC12CC(c3cc([C@H]4C[C@H](OC(F)(F)F)C4)no3)(C1)C2)[C@H]1C[C@@H](O)c2cc(Cl)ccc2O1] 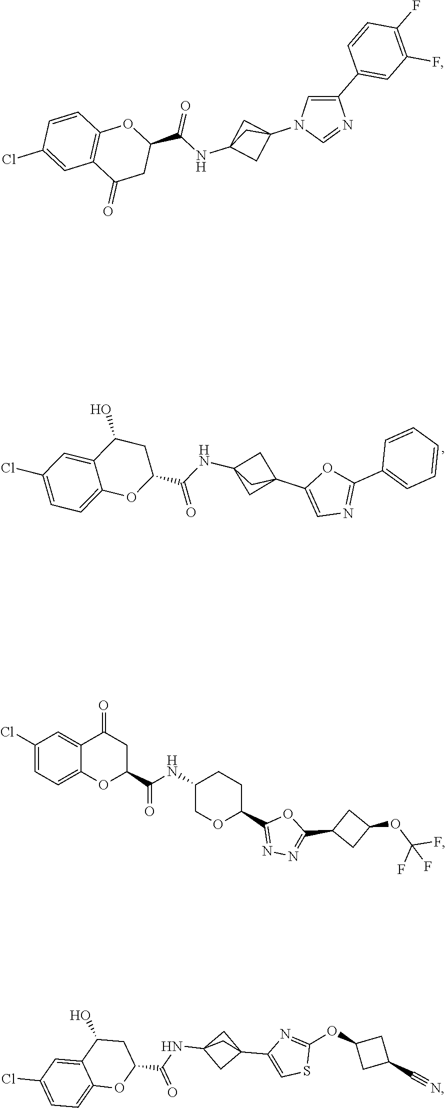 [SMILES: O=C1C[C@H](C(=O)NC23CC(n4cnc(-c5ccc(F)c(F)c5)c4)(C2)C3)Oc2ccc(Cl)cc21 ; O=C(NC12CC(c3cnc(-c4ccccc4)o3)(C1)C2)[C@H]1C[C@@H](O)c2cc(Cl)ccc2O1 ; O=C1C[C@@H](C(=O)N[C@@H]2CC[C@@H](c3nnc([C@H]4C[C@@H](OC(F)(F)F)C4)o3)OC2)Oc2ccc(Cl)cc21 ; N#C[C@H]1C[C@@H](Oc2nc(C34CC(NC(=O)[C@H]5C[C@@H](O)c6cc(Cl)ccc6O5)(C3)C4)cs2)C1] 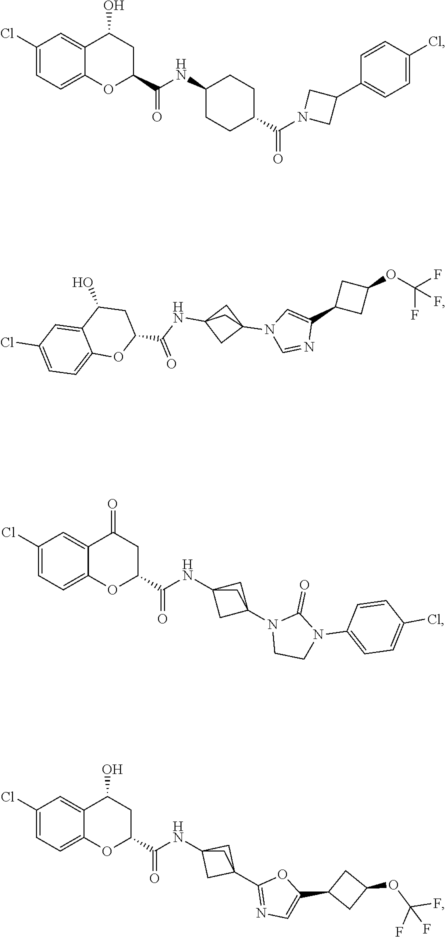 [SMILES: O=C(N[C@H]1CC[C@H](C(=O)N2CC(c3ccc(Cl)cc3)C2)CC1)[C@@H]1C[C@@H](O)c2cc(Cl)ccc2O1 ; O=C(NC12CC(n3cnc([C@H]4C[C@@H](OC(F)(F)F)C4)c3)(C1)C2)[C@H]1C[C@@H](O)c2cc(Cl)ccc2O1 ; O=C1C[C@H](C(=O)NC23CC(N4CCN(c5ccc(Cl)cc5)C4=O)(C2)C3)Oc2ccc(Cl)cc21 ; O=C(NC12CC(c3ncc([C@H]4C[C@@H](OC(F)(F)F)C4)o3)(C1)C2)[C@H]1C[C@@H](O)c2cc(Cl)ccc2O1] 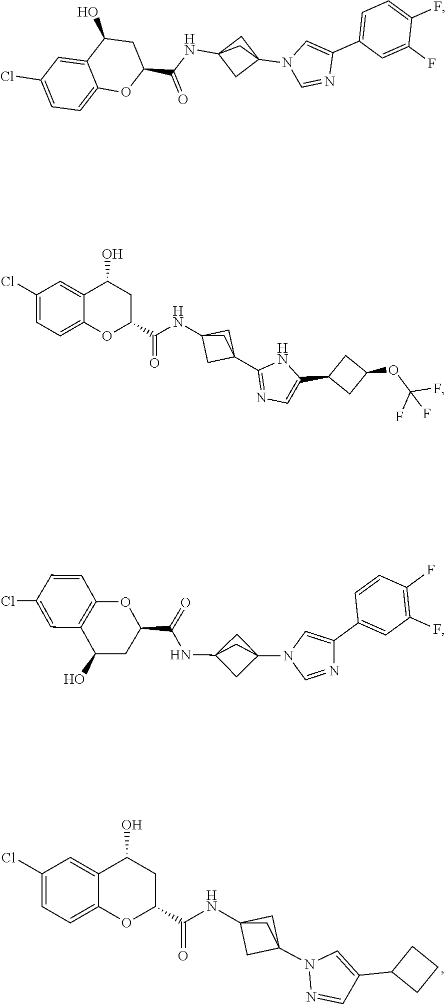 [SMILES: O=C(NC12CC(n3cnc(-c4ccc(F)c(F)c4)c3)(C1)C2)[C@@H]1C[C@H](O)c2cc(Cl)ccc2O1 ; O=C(NC12CC(c3ncc([C@H]4C[C@@H](OC(F)(F)F)C4)[nH]3)(C1)C2)[C@H]1C[C@@H](O)c2cc(Cl)ccc2O1 ; O=C(NC12CC(n3cnc(-c4ccc(F)c(F)c4)c3)(C1)C2)[C@H]1C[C@@H](O)c2cc(Cl)ccc2O1 ; O=C(NC12CC(n3cc(C4CCC4)cn3)(C1)C2)[C@H]1C[C@@H](O)c2cc(Cl)ccc2O1] 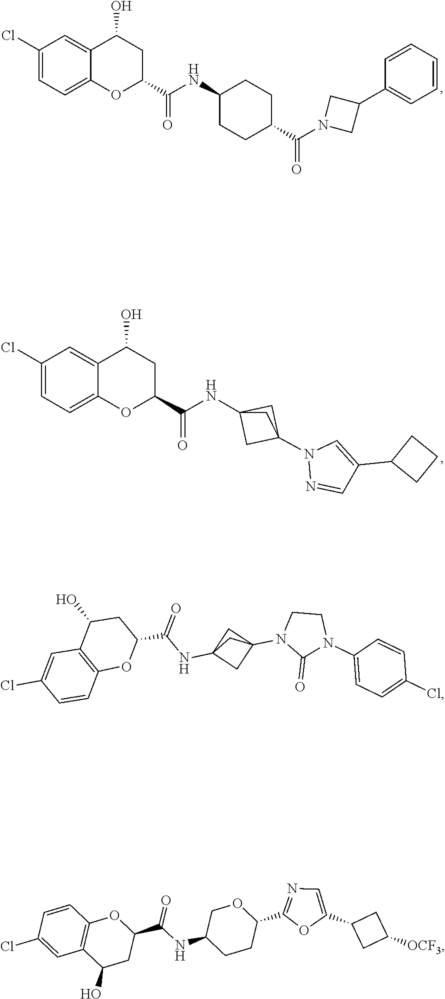 [SMILES: O=C(N[C@H]1CC[C@H](C(=O)N2CC(c3ccccc3)C2)CC1)[C@H]1C[C@@H](O)c2cc(Cl)ccc2O1 ; O=C(NC12CC(n3cc(C4CCC4)cn3)(C1)C2)[C@@H]1C[C@@H](O)c2cc(Cl)ccc2O1 ; O=C(NC12CC(N3CCN(c4ccc(Cl)cc4)C3=O)(C1)C2)[C@H]1C[C@@H](O)c2cc(Cl)ccc2O1 ; O=C(N[C@@H]1CC[C@@H](c2ncc([C@H]3C[C@@H](OC(F)(F)F)C3)o2)OC1)[C@H]1C[C@@H](O)c2cc(Cl)ccc2O1] 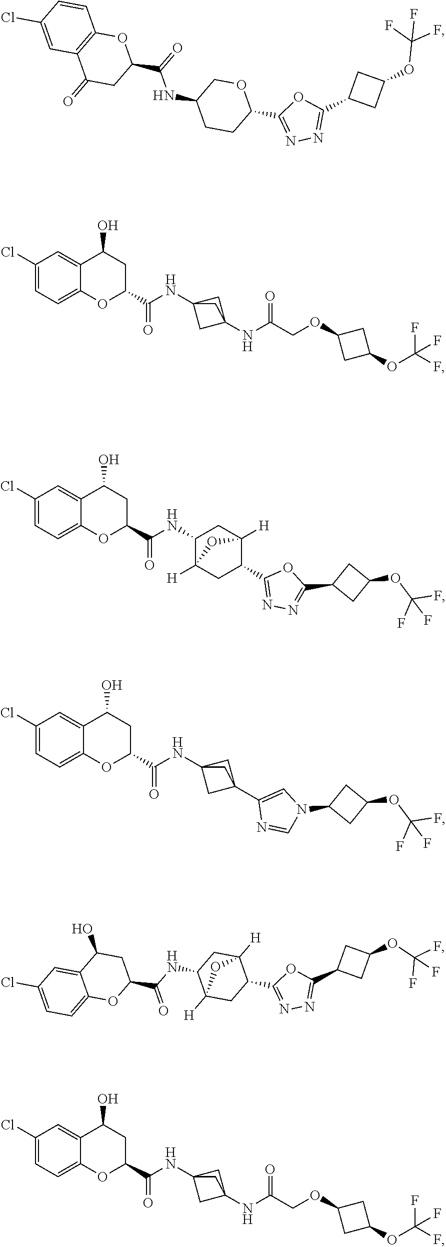 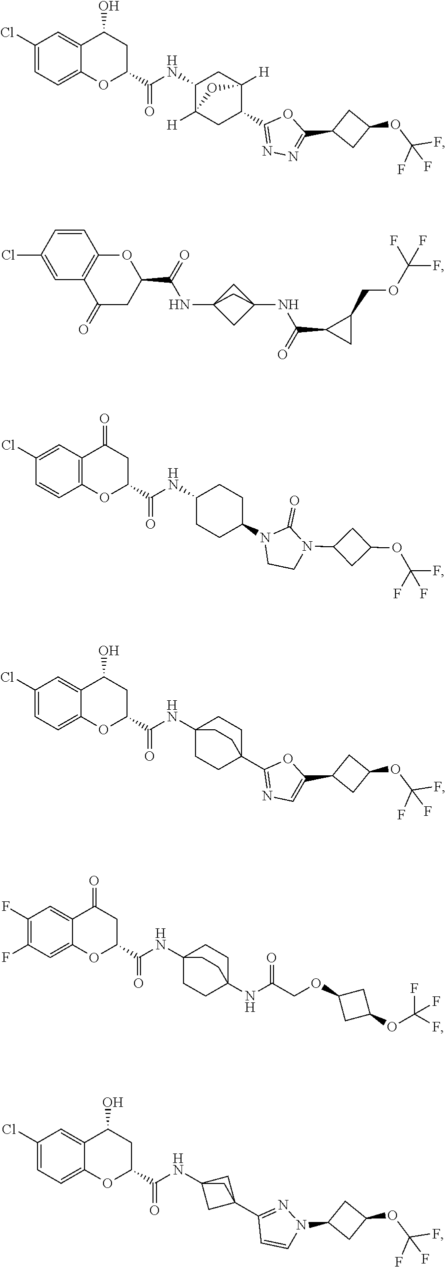 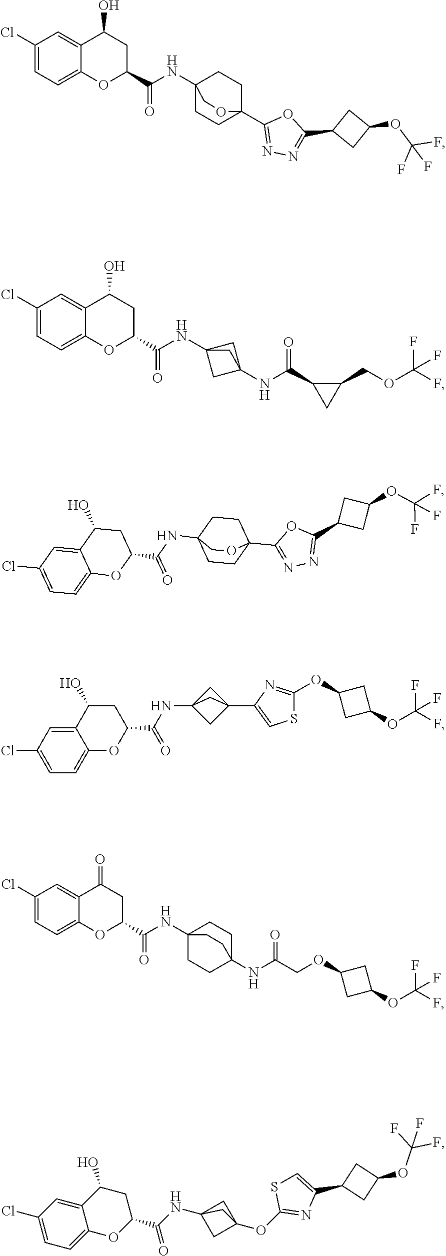 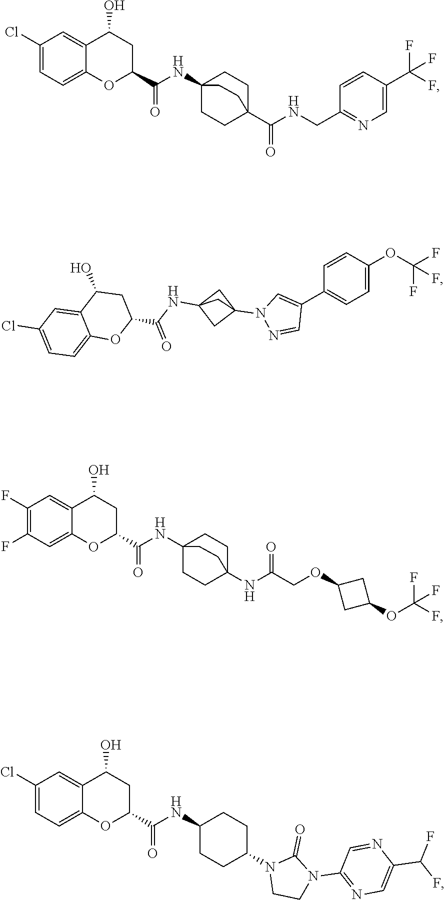 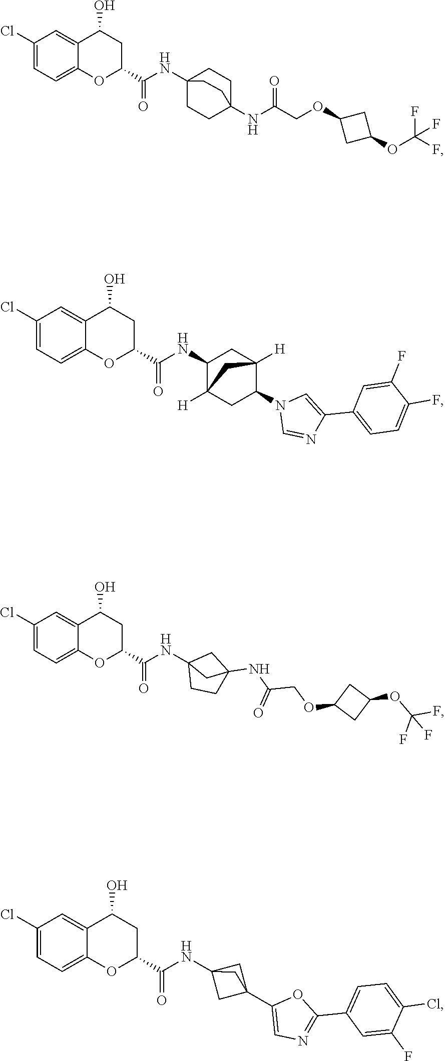 [SMILES: O=C(CO[C@H]1C[C@@H](OC(F)(F)F)C1)NC12CCC(NC(=O)[C@H]3C[C@@H](O)c4cc(Cl)ccc4O3)(CC1)CC2 ; O=C(N[C@H]1C[C@H]2C[C@@H]1C[C@@H]2n1cnc(-c2ccc(F)c(F)c2)c1)[C@H]1C[C@@H](O)c2cc(Cl)ccc2O1 ; O=C(CO[C@H]1C[C@@H](OC(F)(F)F)C1)NC12CCC(NC(=O)[C@H]3C[C@@H](O)c4cc(Cl)ccc4O3)(C1)C2 ; O=C(NC12CC(c3cnc(-c4ccc(Cl)c(F)c4)o3)(C1)C2)[C@H]1C[C@@H](O)c2cc(Cl)ccc2O1] 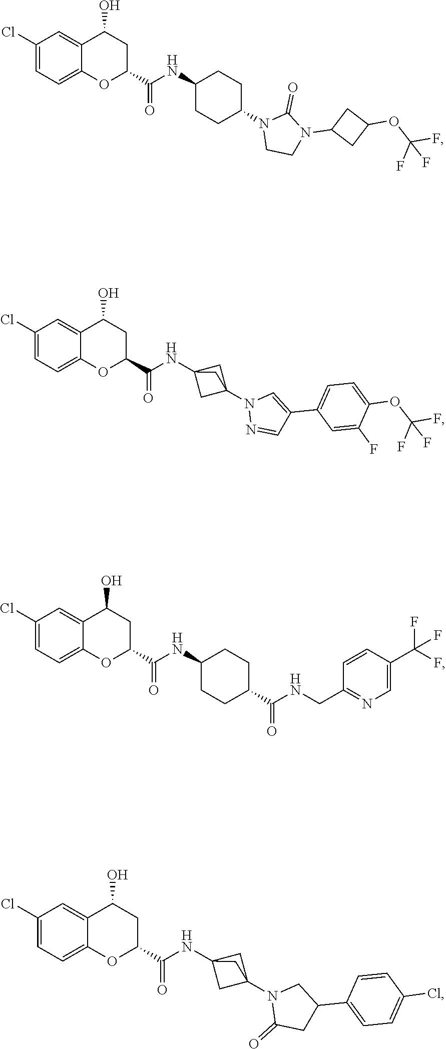 [SMILES: O=C(N[C@H]1CC[C@H](N2CCN(C3CC(OC(F)(F)F)C3)C2=O)CC1)[C@H]1C[C@@H](O)c2cc(Cl)ccc2O1 ; O=C(NC12CC(n3cc(-c4ccc(OC(F)(F)F)c(F)c4)cn3)(C1)C2)[C@@H]1C[C@@H](O)c2cc(Cl)ccc2O1 ; O=C(N[C@H]1CC[C@H](C(=O)NCc2ccc(C(F)(F)F)cn2)CC1)[C@H]1C[C@H](O)c2cc(Cl)ccc2O1 ; O=C(NC12CC(N3CC(c4ccc(Cl)cc4)CC3=O)(C1)C2)[C@H]1C[C@@H](O)c2cc(Cl)ccc2O1] 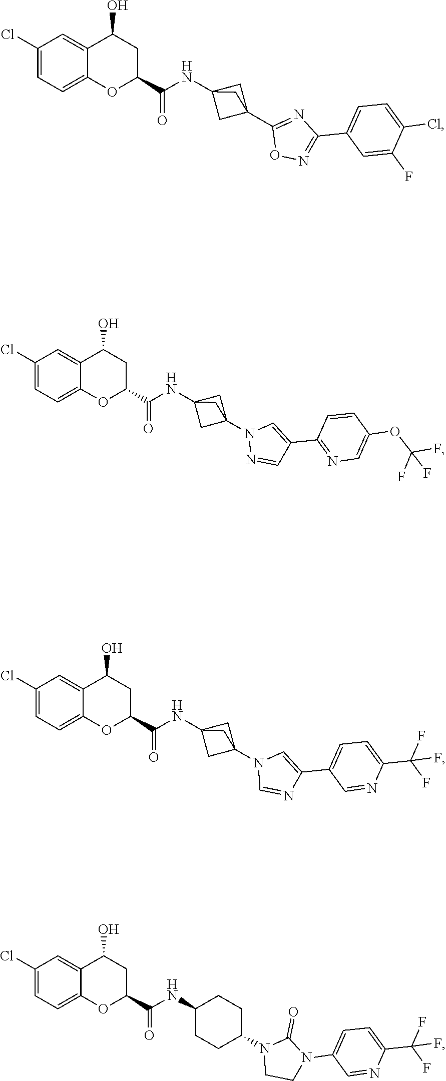 [SMILES: O=C(NC12CC(c3nc(-c4ccc(Cl)c(F)c4)no3)(C1)C2)[C@@H]1C[C@H](O)c2cc(Cl)ccc2O1 ; O=C(NC12CC(n3cc(-c4ccc(OC(F)(F)F)cn4)cn3)(C1)C2)[C@H]1C[C@@H](O)c2cc(Cl)ccc2O1 ; O=C(NC12CC(n3cnc(-c4ccc(C(F)(F)F)nc4)c3)(C1)C2)[C@@H]1C[C@H](O)c2cc(Cl)ccc2O1 ; O=C(N[C@H]1CC[C@H](N2CCN(c3ccc(C(F)(F)F)nc3)C2=O)CC1)[C@@H]1C[C@@H](O)c2cc(Cl)ccc2O1] 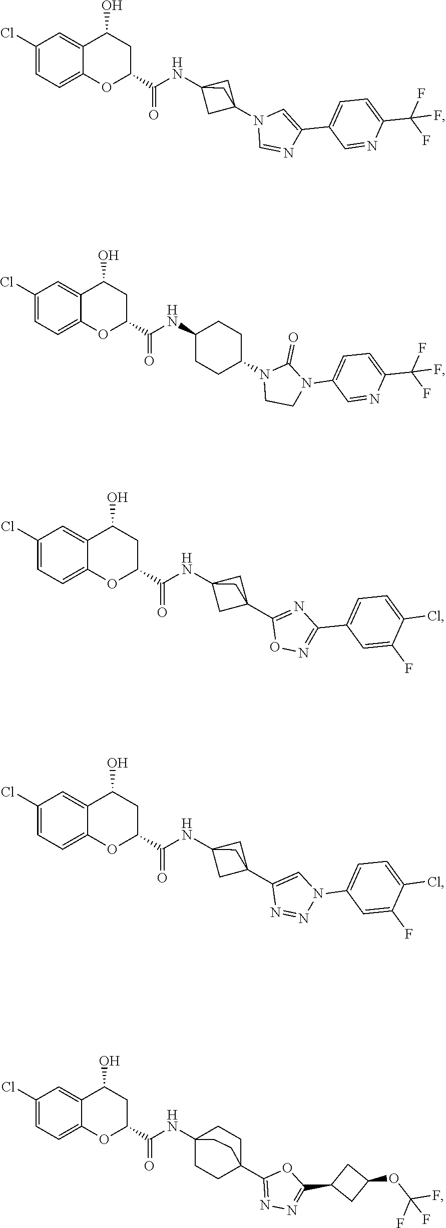 [SMILES: O=C(NC12CC(n3cnc(-c4ccc(C(F)(F)F)nc4)c3)(C1)C2)[C@H]1C[C@@H](O)c2cc(Cl)ccc2O1 ; O=C(N[C@H]1CC[C@H](N2CCN(c3ccc(C(F)(F)F)nc3)C2=O)CC1)[C@H]1C[C@@H](O)c2cc(Cl)ccc2O1 ; O=C(NC12CC(c3nc(-c4ccc(Cl)c(F)c4)no3)(C1)C2)[C@H]1C[C@@H](O)c2cc(Cl)ccc2O1 ; O=C(NC12CC(c3cn(-c4ccc(Cl)c(F)c4)nn3)(C1)C2)[C@H]1C[C@@H](O)c2cc(Cl)ccc2O1 ; O=C(NC12CCC(c3nnc([C@H]4C[C@@H](OC(F)(F)F)C4)o3)(CC1)CC2)[C@H]1C[C@@H](O)c2cc(Cl)ccc2O1] 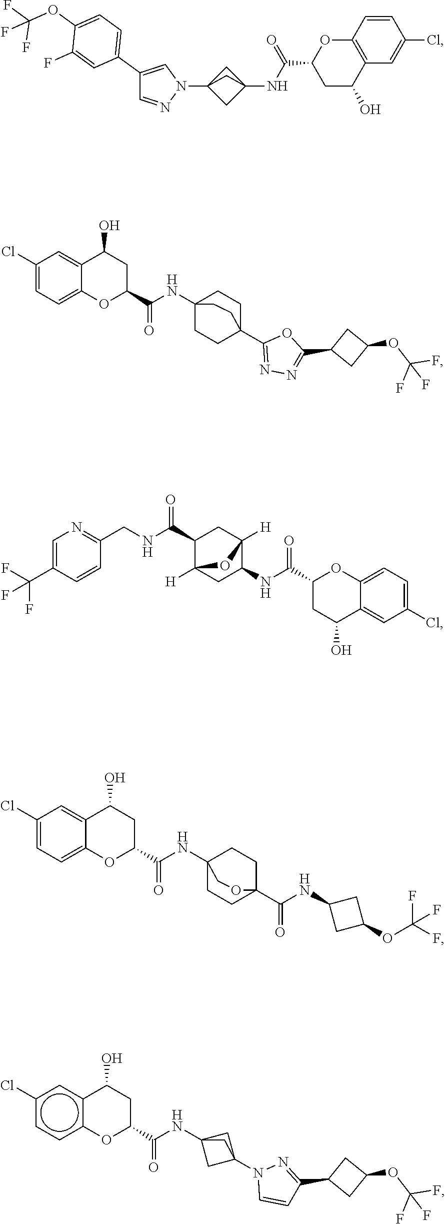 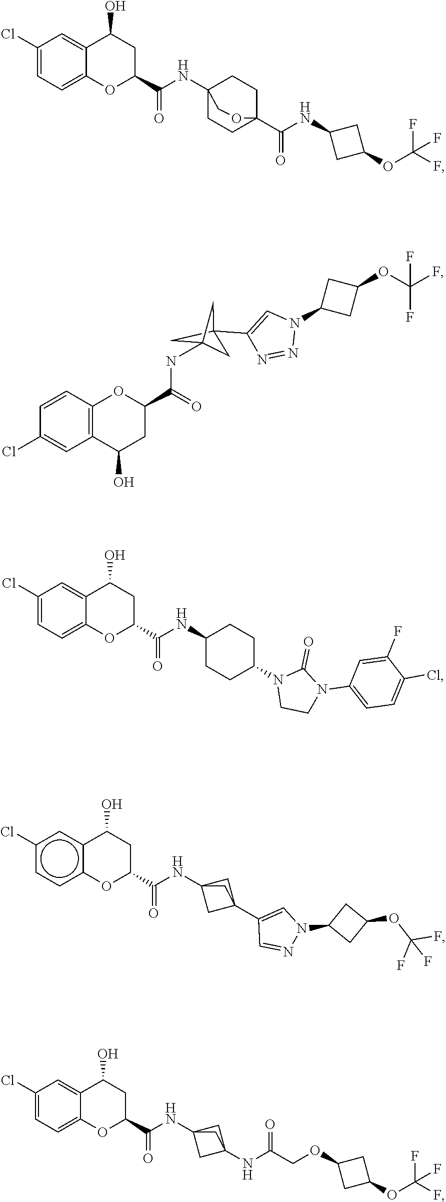 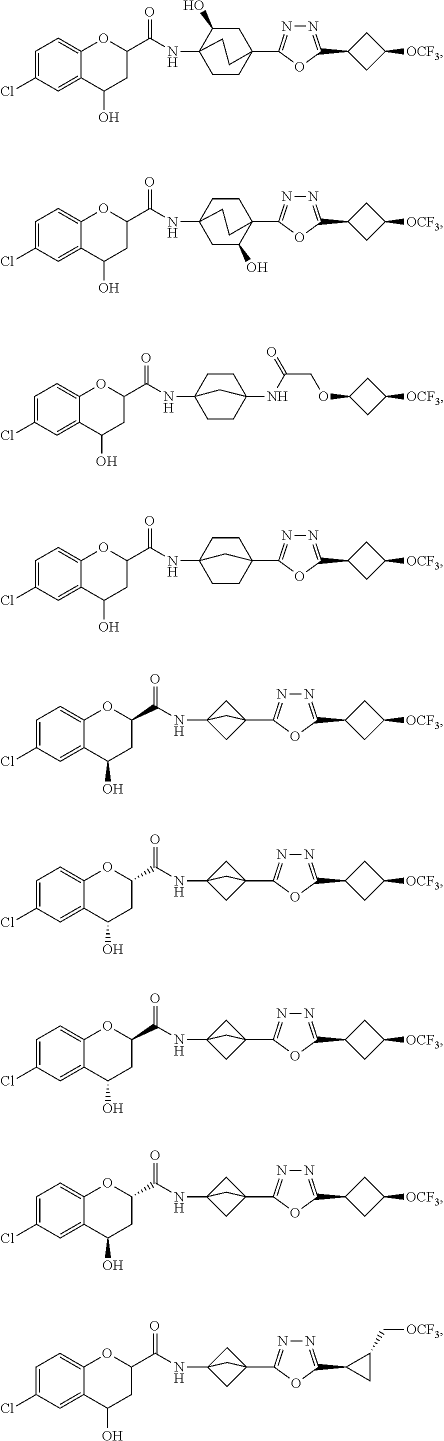 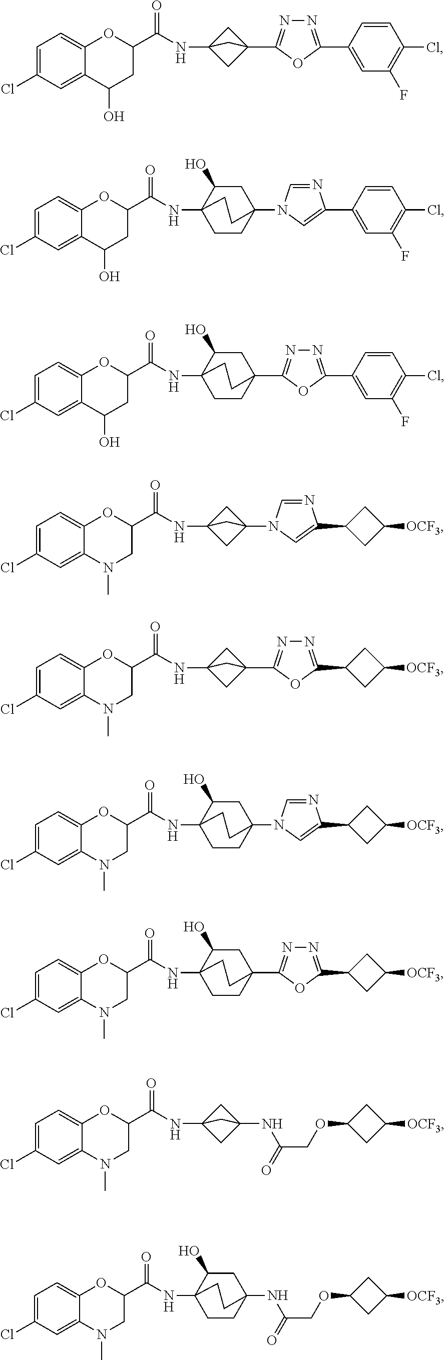 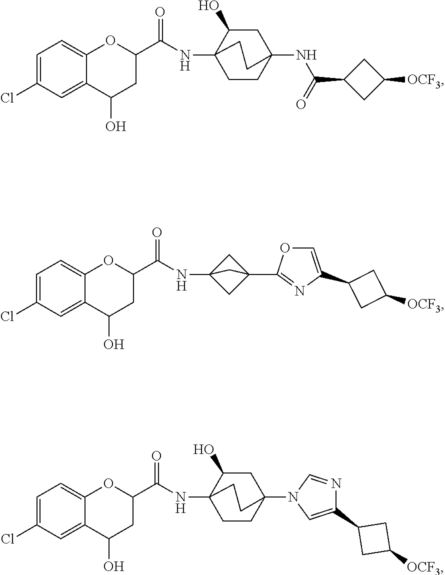 [SMILES: O=C(NC12CCC(NC(=O)[C@H]3C[C@@H](OC(F)(F)F)C3)(CC1)C[C@@H]2O)C1CC(O)c2cc(Cl)ccc2O1 ; O=C(NC12CC(c3nc([C@H]4C[C@@H](OC(F)(F)F)C4)co3)(C1)C2)C1CC(O)c2cc(Cl)ccc2O1 ; O=C(NC12CCC(n3cnc([C@H]4C[C@@H](OC(F)(F)F)C4)c3)(CC1)C[C@@H]2O)C1CC(O)c2cc(Cl)ccc2O1]
and a pharmaceutically acceptable salt, solvate, hydrate, tautomer, N-oxide, or stereoisomer thereof.

75. A pharmaceutically acceptable composition comprising a compound of any one of claims 1-0 and a pharmaceutically acceptable carrier.

76. A method of treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, or a mitochondrial disease in a subject in need thereof, comprising administering to the subject a therapeutically effective amount of a compound of any one of claims 1-0, or a pharmaceutically acceptable salt, solvate, hydrate, tautomer, N-oxide, or stereoisomer thereof.

77. The method of claim 76, wherein the neurodegenerative disease comprises a leukodystrophy, a leukoencephalopathy, a hypomyelinating or demyelinating disease, an intellectual disability syndrome, a cognitive impairment, a glial cell dysfunction, or a brain injury.

78. The method of claim 76 or 77, wherein the neurodegenerative disease comprises vanishing white matter disease, childhood ataxia with CNS hypo myelination, Alzheimer's disease, amyotrophic lateral sclerosis, Creutzfeldt-Jakob disease, frontotemporal dementia, Gerstmann-Straussler-Scheinker disease, Huntington's disease, dementia, kuru, multiple sclerosis, Parkinson's disease, or a prion disease.

79. The method of any one of claims 76-78, wherein the neurodegenerative disease comprises vanishing white matter disease.

80. The method of claim 76, wherein the cancer comprises pancreatic cancer, breast cancer, multiple myeloma, or a cancer of the secretory cells.

81. The method of claim 76, wherein the inflammatory disease comprises postoperative cognitive dysfunction, arthritis, systemic lupus erythematosus (SLE), myasthenia gravis, diabetes), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, acne vulgaris, celiac disease, chronic prostatitis, pelvic inflammatory disease, reperfusion injury, sarcoidosis, transplant rejection, interstitial cystitis, atherosclerosis, or atopic dermatitis.

82. The method of claim 76, wherein the musculoskeletal disease comprises muscular dystrophy, multiple sclerosis, amyotropic lateral sclerosis, primary lateral sclerosis, progressive muscular atrophy, progressive bulbar palsy, pseudobulbar palsy, spinal muscular atrophy, progressive spinobulbar muscular atrophy, spinal cord spasticity, spinal muscle atrophy, myasthenia gravis, neuralgia, fibromyalgia, Machado-Joseph disease, cramp fasciculation syndrome, Freidrich's ataxia, a muscle wasting disorder), an inclusion body myopathy, motor neuron disease, or paralysis.

83. The method of claim 76, wherein the metabolic disease comprises non-alcoholic steatohepatitis (NASH), non-alcoholic fatty liver disease (NAFLD), liver fibrosis, obesity, heart disease, atherosclerosis, arthritis, cystinosis, diabetes, phenylketonuria, proliferative retinopathy, or Kearns-Sayre disease.

84. The method of claim 76, wherein the mitochondrial disease is associated with or is a result of mitochondrial dysfunction, one or more mitochondrial protein mutations, or one or more mitochondrial DNA mutations.

85. The method of claim 76 or 84, wherein the mitochondrial disease is a mitochondrial myopathy.

86. The method of any one of claims 76 and 84-85, wherein the mitochondrial disease is selected from the group consisting of Barth syndrome, chronic progressive external ophthalmoplegia (cPEO), Kearns-Sayre syndrome (KSS), Leigh syndrome (e.g., MILS, or maternally inherited Leigh syndrome), mitochondrial DNA depletion syndromes (MDDS, e.g., Alpers syndrome), mitochondrial encephalomyopathy (e.g., mitochondrial encephalomyopathy, lactic acidosis, and stroke-like episodes (MELAS)), mitochondrial neurogastrointestinal encephalomyopathy (MNGIE), myoclonus epilepsy with ragged red fibers (MERRF), neuropathy, ataxia, retinitis pigmentosa (NARP), Leber's hereditary optic neuropathy (LHON) and Pearson syndrome.

87. The method of claim 76, wherein the autoimmune disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Balb disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

88. The method of claim 76, wherein the viral infection is selected from the group consisting of influenza, human immunodeficiency virus (HIV) and herpes.

89. The method of claim 76, wherein the skin disease is selected from the group consisting of acne, alopecia areata, basal cell carcinoma, Bowen's disease, congenital erythropoietic porphyria, contact dermatitis, Darier's disease, disseminated superficial actinic porokeratosis, dystrophic epidermolysis bullosa, eczema (atopic eczema), extra-mammary Paget's disease, epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, mucous membrane pemphigoid, pemphigoid, pemphigus vulgaris, pityriasis lichenoides, pityriasis rubra pilaris, plantar warts (verrucas), polymorphic light eruption, psoriasis, plaque psoriasis, pyoderma gangrenosum, rosacea, scabies, scleroderma, shingles, squamous cell carcinoma, sweet's syndrome, urticaria and angioedema and vitiligo.

90. The method of claim 76, wherein the fibrotic disease is selected from the group consisting of adhesive capsulitis, arterial stiffness, arthrofibrosis, atrial fibrosis, cardiac fibrosis, cirrhosis, congenital hepatic fibrosis, Crohn's disease, cystic fibrosis, Dupuytren's contracture, endomyocardial fibrosis, glial scar, hepatitis C, hypertrophic cardiomyopathy, hypersensitivity pneumonitis, idiopathic pulmonary fibrosis, idiopathic interstitial pneumonia, interstitial lung disease, keloid, mediastinal fibrosis, myelofibrosis, nephrogenic systemic fibrosis, non-alcoholic fatty liver disease, old myocardial infarction, Peyronie's disease, pneumoconiosis, pneumonitis, progressive massive fibrosis, pulmonary fibrosis, radiation-induced lung injury, retroperitoneal fibrosis, scleroderma/systemic sclerosis, silicosis and ventricular remodeling.

91. The method of claim 76, wherein the hemoglobin disease is selected from the group consisting of “dominant” β-thalassemia, acquired (toxic) methemoglobinemia, carboxyhemoglobinemia, congenital Heinz body hemolytic anemia, HbH disease, HbS/β-thalassemia, HbE/β-thalassemia, HbSC disease, homozygous α⁺-thalassemia (phenotype of α⁰-thalassemia), Hydrops fetalis with Hb Bart's, sickle cell anemia/disease, sickle cell trait, sickle β-thalassemia disease, α⁺-thalassemia, α⁰-thalassemia, α-Thalassemia associated with myelodysplastic syndromes, α-Thalassemia with mental retardation syndrome (ATR), β⁰-Thalassemia, β⁺-Thalassemia, δ-Thalassemia, γ-Thalassemia, β-Thalassemia major, β-Thalassemia intermedia, δβ-Thalassemia, and εγδβ-Thalassemia.

92. The method of claim 76, wherein the kidney disease is selected from the group consisting of Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, P-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Induced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1p-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjögren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VGEF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome.

93. The method of claim 76, wherein the hearing loss condition is selected from the group consisting of mitochondrial nonsyndromic hearing loss and deafness, hair cell death, age-related hearing loss, noise-induced hearing loss, genetic or inherited hearing loss, hearing loss experienced as a result of ototoxic exposure, hearing loss resulting from disease, and hearing loss resulting from trauma.

94. The method of claim 76, wherein the ocular disease cataracts, glaucoma, endoplasmic reticulum (ER) stress, autophagy deficiency, age-related macular degeneration (AMD), or diabetic retinopathy.

95. The method of any one of claims 76-94, further comprising a second agent for treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, a mitochondrial disease, or a disease or disorder associated with impaired function of eIF2B, eIF2α, or a component of the eIF2 pathway or ISR pathway.

96. A method of treating a disease related to a modulation of eIF2B activity or levels, eIF2α activity or levels, or the activity or levels of a component of the eIF2 pathway or the ISR pathway in a subject in need thereof, comprising administering to the patient a therapeutically effective amount of any one of claims 1-74, or a pharmaceutically acceptable salt, solvate, hydrate, tautomer, N-oxide, or stereoisomer thereof.

97. The method of claim 96, wherein the modulation comprises an increase in eIF2B activity or levels, increase in eIF2α activity or levels, or increase in activity or levels of a component of the eIF2 pathway or the ISR pathway.

98. The method of claim 96, wherein the disease may be caused by a mutation to a gene or protein sequence related to a member of the eIF2 pathway.

99. A method of treating cancer in a subject in need thereof, comprising administering to the subject a therapeutically effective amount of a compound of any one of claims 1-74, or a pharmaceutically acceptable salt, solvate, hydrate, tautomer, N-oxide, or stereoisomer thereof in combination with an immunotherapeutic agent.
